(19) United States
(12) Patent Application Publication
(10) Pub. No.: US 2025/0302832 A1
(43) Pub. Date: Oct. 2, 2025

(54) PYRAZINE DERIVATIVES AND USES THEREOF

(71) Applicant: FOGHORN THERAPEUTICS INC., Cambridge, MA (US)

(72) Inventors: Matthew NETHERTON, Cambridge, MA (US); Jing DENG, Cambridge, MA (US); Francois BRUCELLE, Belmont, MA (US); Johannes H. VOIGT, Cambridge, MA (US)

(21) Appl. No.: 18/863,946

(22) PCT Filed: May 10, 2023
(86) PCT No.: PCT/US2023/021671

§ 371 Date: Nov. 7, 2024

Related U.S. Application Data
(60) Provisional application No. 63/340,275, filed on May 10, 2022.

(51) Int. Cl. A61K 31/5025; A61K 31/506; C07D 487/04

(52) U.S. Cl. CPC A61K 31/5025; A61K 31/506; C07D 487/04

(57) ABSTRACT

The present disclosure features compounds of Formula I, or pharmaceutically acceptable salts thereof, and formulations containing the same. Methods of treating BAF complex-related disorders, such as cancer, are also disclosed.

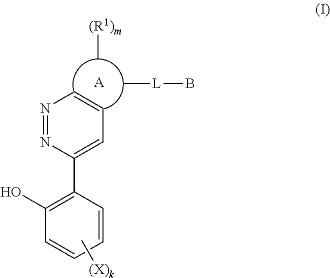

### BACKGROUND

The invention relates to compounds useful for modulating BRG1- or BRM-associated factors (BAF) complexes. In particular, the invention relates to compounds useful for treatment of disorders associated with BAF complex function.

Chromatin regulation is essential for gene expression, and ATP-dependent chromatin remodeling is a mechanism by which such gene expression occurs. The human Switch/Sucrose Non-Fermentable (SWI/SNF) chromatin remodeling complex, also known as BAF complex, has two SWI2-like ATPases known as BRG1 (Brahma-related gene-1) and BRM (Brahma). The transcription activator BRG1, also known as ATP-dependent chromatin remodeler SMARCA4, is encoded by the SMARCA4 gene on chromosome 19. BRG1 is overexpressed in some cancer tumors and is needed for cancer cell proliferation. BRM, also known as probable global transcription activator SNF2L2 and/or ATP-dependent chromatin remodeler SMARCA2, is encoded by the SMARCA2 gene on chromosome 9 and has been shown to be essential for tumor cell growth in cells characterized by loss of BRG1 function mutations. Deactivation of BRG and/or BRM results in downstream effects in cells, including cell cycle arrest and tumor suppression.

### SUMMARY

The present invention features compounds useful for modulating a BAF complex. In some embodiments, the compounds are useful for the treatment of disorders associated with an alteration in a BAF complex, e.g., a disorder associated with an alteration in one or both of the BRG1 and BRM proteins. The compounds of the invention, alone or in combination with other pharmaceutically active agents, can be used for treating such disorders.

In an aspect, the invention features a compound, or a pharmaceutically acceptable salt thereof, having the structure of Formula I:

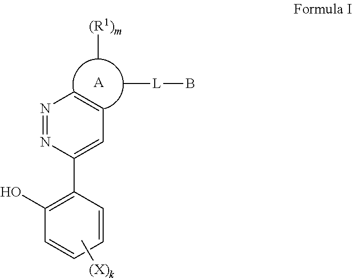

where
ring system A is a 5 to 9-membered heterocyclyl or heteroaryl containing at least one N;
m is 0, 1, 2, or 3;
k is 0, 1, or 2;
each R¹ is, independently, halo, optionally substituted C₁-C₆ alkyl, optionally substituted C₁-C₆ heteroalkyl, optionally substituted C₂-C₉ heterocyclyl, optionally substituted C₃-C₈ cycloalkyl or optionally substituted CH₂—C₃-C₈ cycloalkyl;
each X is, independently, halo;
L is a linker; and
B is a degradation moiety.

In some embodiments, the compound has the structure of Formula I-A:

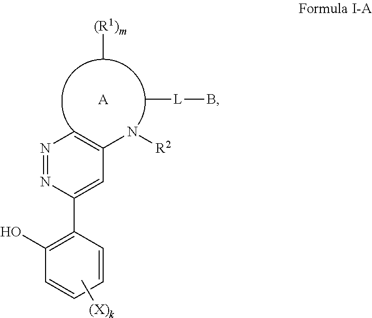

where R² is H, optionally substituted C₁-C₆ alkyl, optionally substituted C₃-C₈ cycloalkyl, optionally substituted C₂-C₉ heterocyclyl, or a bond to -L-B.

In some embodiments, the compound has the structure of Formula I-B

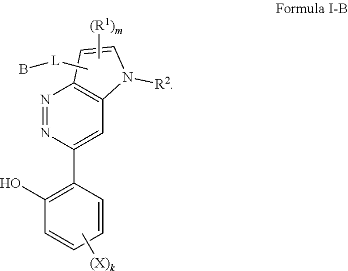

In some embodiments, the compound has the structure of Formula I-C

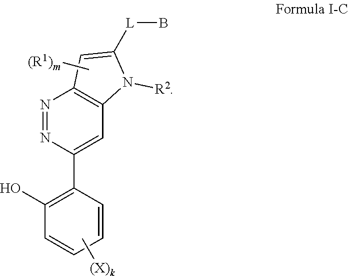

In some embodiments, k is 0. In some embodiments, k is 1. In some embodiments, k is 2. In some embodiments, X is Cl or F.

In some embodiments, m is 0. In some embodiments m is 1. In some embodiments R1 is optionally substituted C₁-C₆ alkyl. In some embodiments, R¹ is methyl. In some embodiments, R¹ is difluoromethyl.

In some embodiments, R² is H. In some embodiments, R² is optionally substituted C₁-C₆ alkyl. In some embodiments, R² is optionally substituted C₃-C₈ cycloalkyl (e.g., monocyclic, spiro, bridged). In some embodiments, R² is optionally substituted C₂-C₉ heterocyclyl. In some embodiments, R² is H, CH₃,

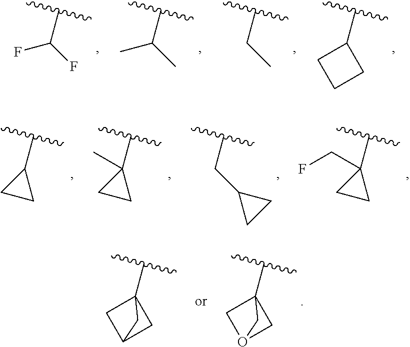

In some embodiments, the compound has the structure of Formula I-D:

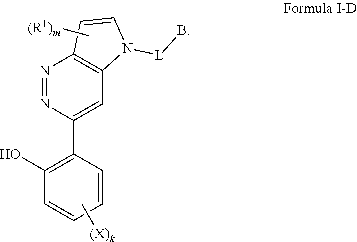

In some embodiments, m is 0. In some embodiments, m is 1. In some embodiments, m is 2.

In some embodiments, R¹ is methyl or difluoromethyl. In some embodiments, k is 0.

In some embodiments, the compound has the structure of formula I-E:

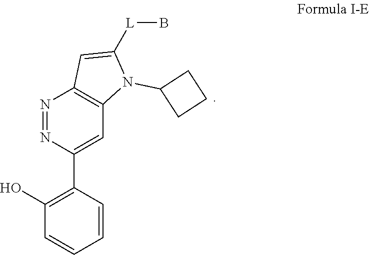

In some embodiments, the compound has the structure of formula I-F:

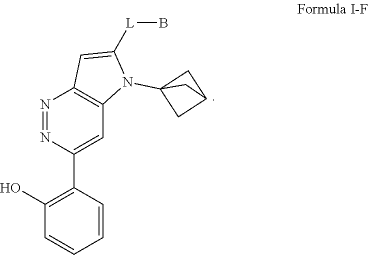

In some embodiments, the compound has the structure of formula I-G:

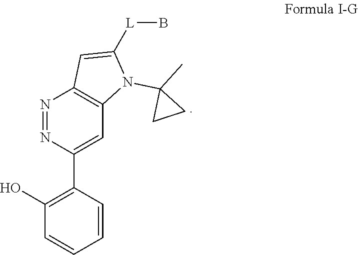

In some embodiments, the compound has the structure of formula I-H:

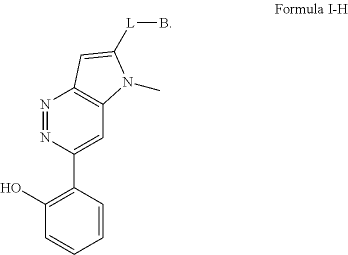

In some embodiments, the compound has the structure of formula I-I:

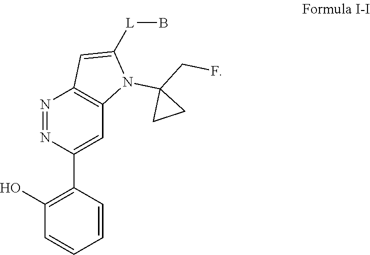

In some embodiments, the compound has the structure of formula I-J:

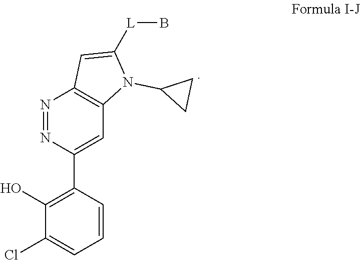

In some embodiments, the compound has the structure of formula I-K:

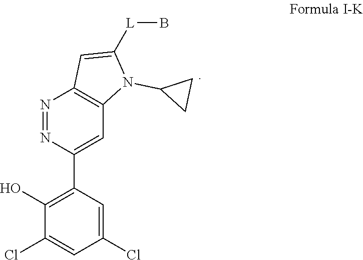

In some embodiments, the compound has the structure of formula I-L:

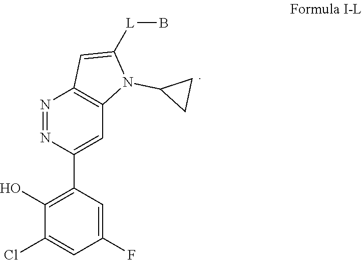

In some embodiments, the compound has the structure of formula I-M:

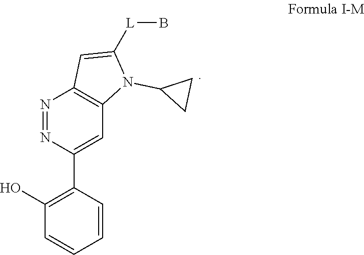

In some embodiments, the compound has the structure of formula I-N:

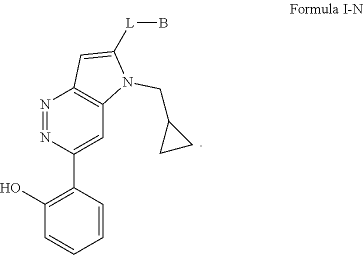

In some embodiments, the compound has the structure of formula I-O:

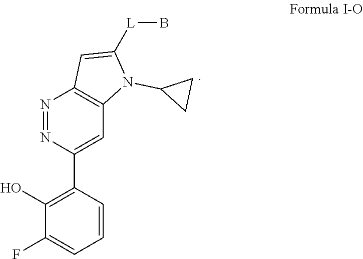

In some embodiments, the compound has the structure of formula I-P:

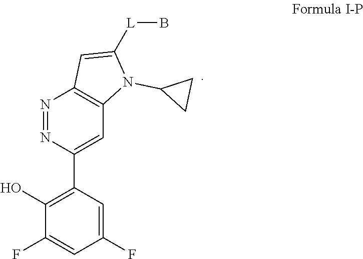

In some embodiments, the compound has the structure of formula I-Q:

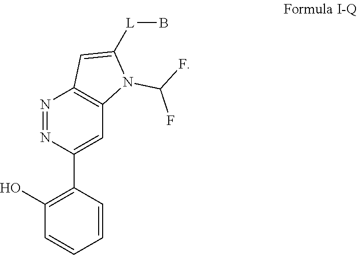

In some embodiments, the compound has the structure of formula I-R:

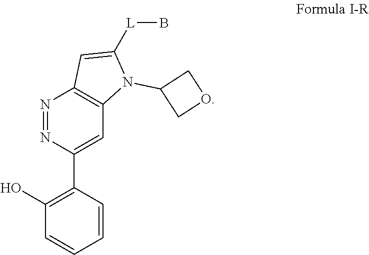

In some embodiments, the compound has the structure of formula I-S:

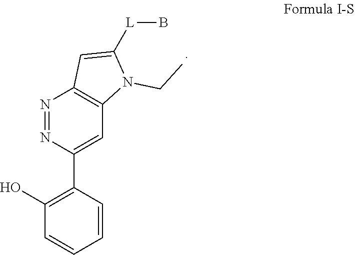

In some embodiments, the compound has the structure of formula I-T:

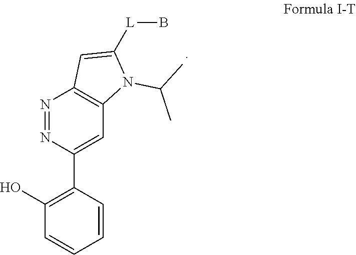

In some embodiments, the compound has the structure of formula I-U:

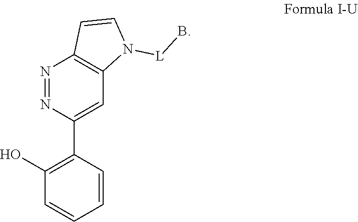

In some embodiments, the compound has the structure of formula I-V:

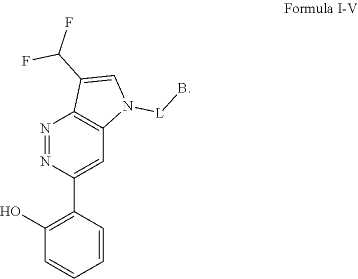

In some embodiments, the compound has the structure of formula I-W:

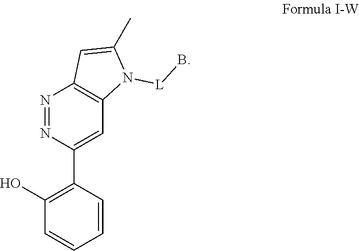

In some embodiments, the degradation moiety, B, has the structure of Formula A-1:

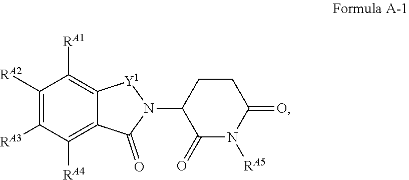

where
Y¹ is

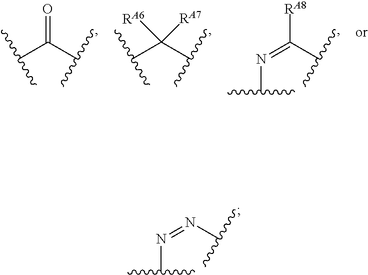

R^{A5} is H, optionally substituted C₁-C₆ alkyl, or optionally substituted C₁-C₆ heteroalkyl;
R^{A6} is H or optionally substituted C₁-C₆ alkyl; and R^{A7} is H or optionally substituted C₁-C₆ alkyl; or R^{A6} and R^{A7}, together with the carbon atom to which each is bound, combine to form optionally substituted C₃-C₆ carbocyclyl or optionally substituted C₂-C₅ heterocyclyl; or R^{A6} and R^{A7}, together with the carbon atom to which each is bound, combine to form optionally substituted C₃-C₆ carbocyclyl or optionally substituted C₂-C₅ heterocyclyl;
R^{A8} is H, optionally substituted C₁-C₆ alkyl, or optionally substituted C₁-C₆ heteroalkyl;
each of R^{A1}, R^{A2}, R^{A3} and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

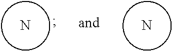

and is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A²,
where one of R^{A1}, R^{A2}, R^{A3}, and R^{A4} is A², or

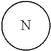

is substituted with A²; and
A² is a bond between the degradation moiety and the linker.

In some embodiments, R^{A5} is H or methyl. In some embodiments, R^{A5} is H.

In some embodiments, each of R^{A1}, R^{A2}, R^{A3}, and R^{A4} is, independently, H or A².

In some embodiments, R^{A1} is A² and each of R^{A2}, R^{A3}, and R^{A4} is H.

In some embodiments, R^{A2} is A² and each of R^{A1}, R^{A3}, and R^{A4} is H.

In some embodiments, R^{A3} is A² and each of R^{A1}, R^{A2} and R^{A4} is H.

In some embodiments, R^{A4} is A² and each of R^{A1}, R^{A2} and R^{A3} is H.

In some embodiments, Y¹ is

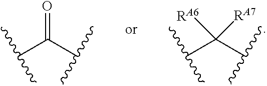

In some embodiments, R^{A6} is H. In some embodiments, R^{A7} is H.

In some embodiments, Y¹ is

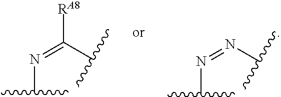

In some embodiments, R^{A8} is H or optionally substituted C₁-C₆ alkyl. In some embodiments, R^{A8} is H or methyl. In some embodiments, R^{A8} is methyl.

In some embodiments, the degradation moiety includes the structure of Formula A2:

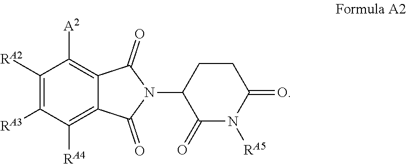

In some embodiments, the degradation moiety is

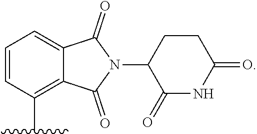 [SMILES: *c1cccc2c1C(=O)N(C1CCC(=O)NC1=O)C2=O]

In some embodiments, the degradation moiety includes the structure of Formula A4:

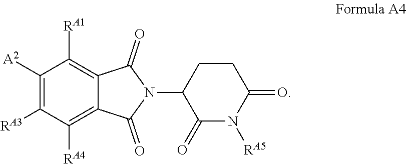

In some embodiments, the degradation moiety is

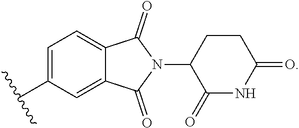 [SMILES: *c1ccc2c(c1)C(=O)N(C1CCC(=O)NC1=O)C2=O]

In some embodiments, the degradation moiety has the structure of Formula A5:

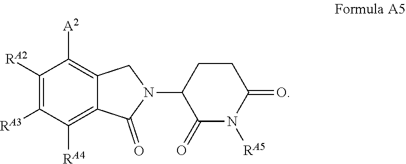

In some embodiments, the degradation moiety has the structure of Formula A6:

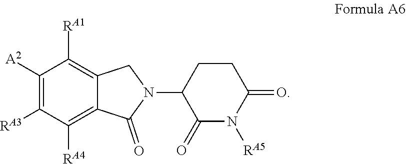

In some embodiments, the degradation moiety has the structure of Formula A8:

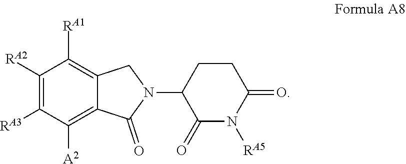

In some embodiments, the degradation moiety has the structure of Formula A10:

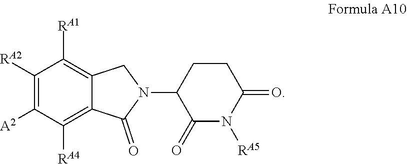

In some embodiments, the degradation moiety has the structure of

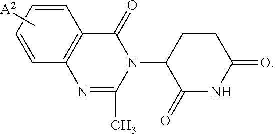

In some embodiments, the degradation moiety has the structure of

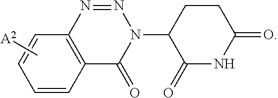

In some embodiments, the degradation moiety has the structure of Formula C:

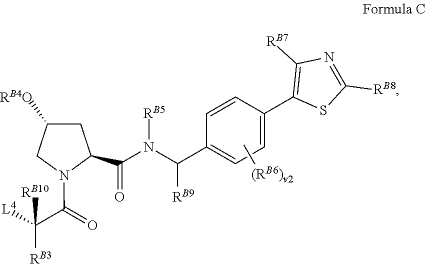

where
L⁴ is —N(R^{B1})(R^{B2}),

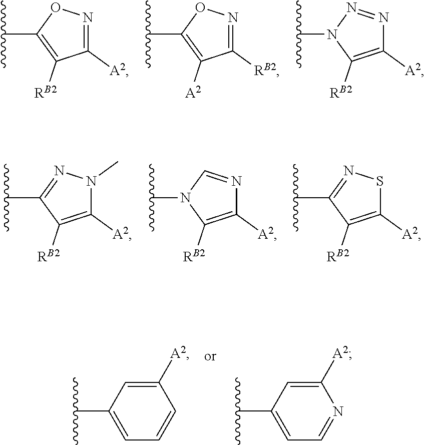

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₂-C₆ alkynyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl; and
A² is a bond between the degradation moiety and the linker;
where one and only one of R^{B1}, R^{B3}, and R^{B6} is A²,
or a pharmaceutically acceptable salt thereof.

In some embodiments, the degradation moiety has the structure of Formula C:

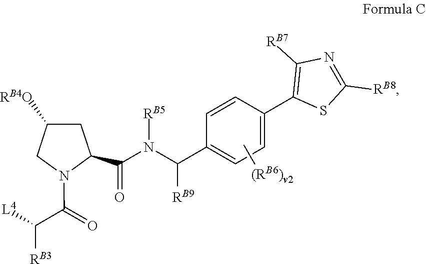

where
L⁴ is —N(R^{B1})(R^{B2}),

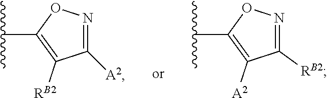

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each of R^{B7} and R^{B8} is, independently, H, halogen, optionally substituted C₁-C₆ alkyl, or optionally substituted C₆-C₁ aryl;
R^{B9} is H or optionally substituted C₁-C₆ alkyl;
R^{B10} is H or F; and
A² is a bond between the degradation moiety and the linker;
where one and only one of R^{B1}, R^{B3}, and R^{B6} is A²,
   or a pharmaceutically acceptable salt thereof.

In some embodiments, the degradation moiety has the structure of Formula C3

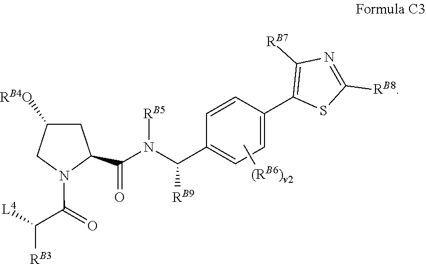

In some embodiments, the degradation moiety has the structure of Formula C4.

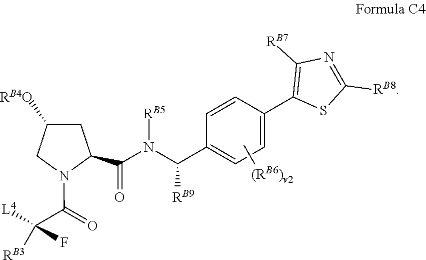

In some embodiments, the degradation moiety has the structure of Formula C1:

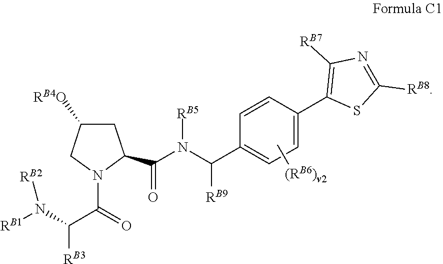

In some embodiments, the degradation moiety is

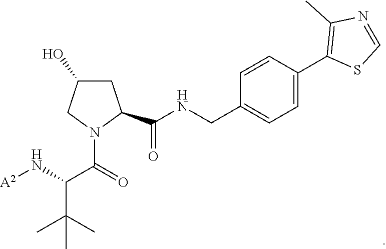

In some embodiments, the degradation moiety is

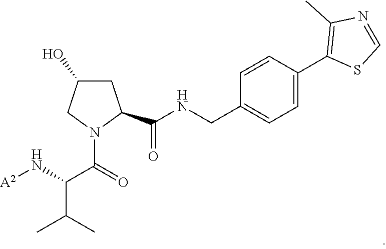

In some embodiments, the degradation moiety is

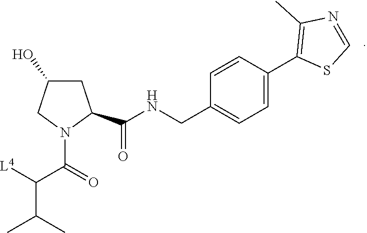

In some embodiments, the degradation moiety

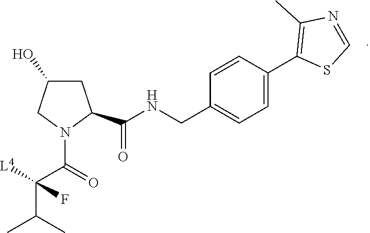

In some embodiments, the degradation moiety is

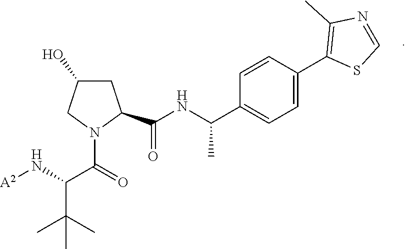

In some embodiments, the degradation moiety is

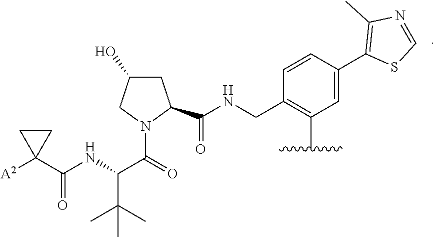

In some embodiments, the degradation moiety has the structure of Formula C2:

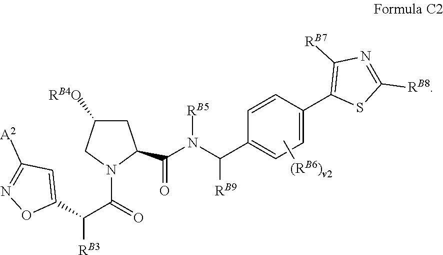

In some embodiments, R^{B9} is optionally substituted C₁-C₆ alkyl. In some embodiments, R^{B9} is methyl.

In some embodiments, R^{B9} is bonded to (S)-stereogenic center.

In some embodiments, v2 is 0. In some embodiments, R^{B4} is H. In some embodiments, R^{B5} is H. In some embodiments, R^{B7} is optionally substituted C₁-C₆ alkyl. In some embodiments, R^{B7} is methyl. In some embodiments, R^{B3} is optionally substituted C₁-C₆ alkyl. In some embodiments, R^{B3} is isopropyl. In some embodiments, R^{B8} is H. In some embodiments, R^{B2} is H.

In some embodiments, the degradation moiety is

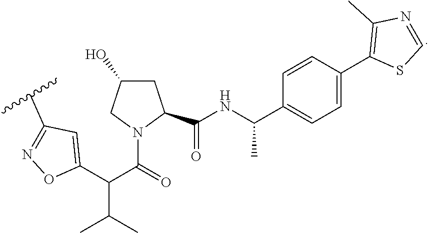 [SMILES: *c1cc(C(C(=O)N2C[C@H](O)C[C@H]2C(=O)N[C@@H](C)c2ccc(-c3scnc3C)cc2)C(C)C)on1]

In some embodiments, the degradation moiety has the structure of Formula Ca2:

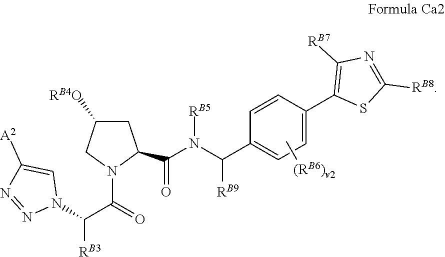

In some embodiments, the degradation moiety has the structure of Formula Cb2:

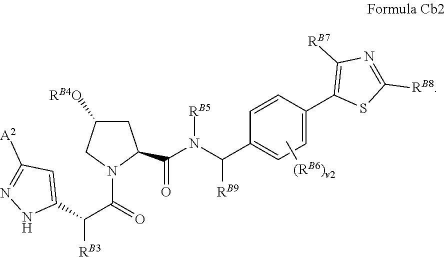

In some embodiments, the degradation moiety has the structure of Formula Cc2:

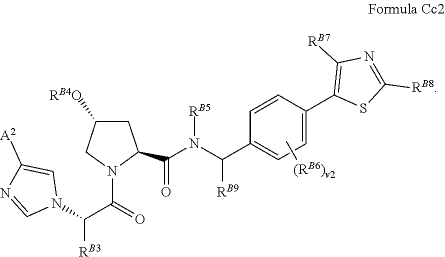

In some embodiments, the degradation moiety has the structure of Formula Cd2:

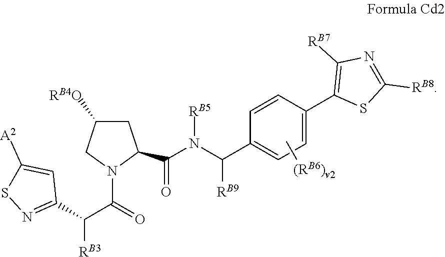

In some embodiments, the degradation moiety has the structure of Formula Ce2:

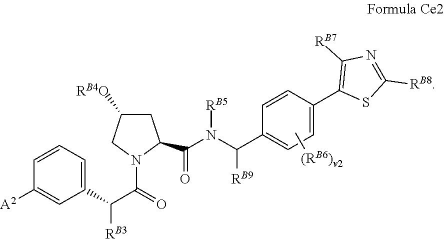

In some embodiments, the degradation moiety has the structure of Formula Cf2:

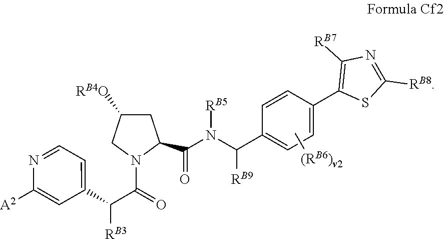

In some embodiments, R^{B9} is optionally substituted C₁-C₆ alkyl. In some embodiments, R^{B9} is methyl.

In some embodiments, R^{B9} is bonded to (S)-stereogenic center.

In some embodiments, v2 is 0. In some embodiments, R^{B4} is H. In some embodiments, R^{B5} is H. In some embodiments, R^{B7} is optionally substituted C₁-C₆ alkyl. In some embodiments, R^{B7} is methyl. In some embodiments, R^{B3} is optionally substituted C₁-C₆ alkyl. In some embodiments, R^{B3} is isopropyl. In some embodiments, R^{B3} is optionally substituted C₃-C₁₀ carbocyclyl. In some embodiments, R^{B3} is cyclopropane. In some embodiments, R^{B3} is cyclobutane. In some embodiments, R^{B3} is fluoro-2-methylpropane. In some embodiments, R^{B8} is H. In some embodiments, R^{B2} is H.

In some embodiments, the degradation moiety is

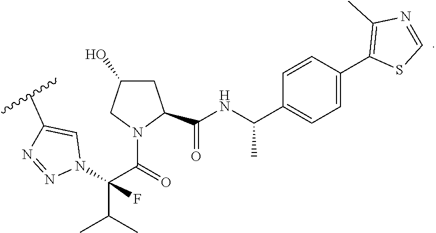

In some embodiments, the degradation moiety is

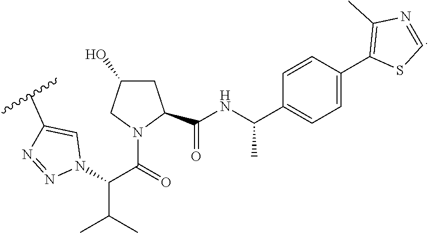 [SMILES: *c1cn([C@H](C(=O)N2C[C@H](O)C[C@H]2C(=O)N[C@@H](C)c2ccc(-c3scnc3C)cc2)C(C)C)nn1]

In some embodiments, the degradation moiety is

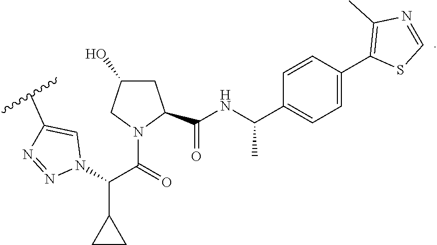 [SMILES: *c1cn([C@H](C(=O)N2C[C@H](O)C[C@H]2C(=O)N[C@@H](C)c2ccc(-c3scnc3C)cc2)C2CC2)nn1]

In some embodiments, the degradation moiety is

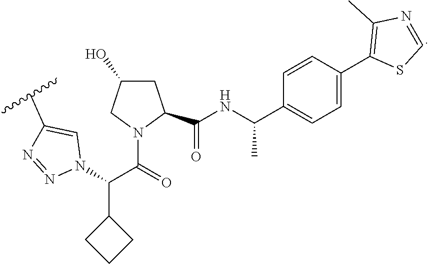 [SMILES: *c1cn([C@H](C(=O)N2C[C@H](O)C[C@H]2C(=O)N[C@@H](C)c2ccc(-c3scnc3C)cc2)C2CCC2)nn1]

In some embodiments, the degradation moiety is

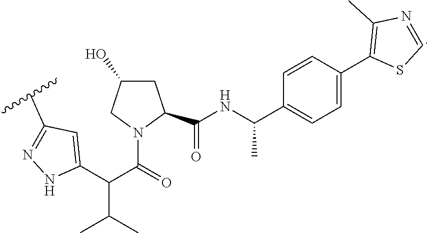 [SMILES: *c1cc(C(C(=O)N2C[C@H](O)C[C@H]2C(=O)N[C@@H](C)c2ccc(-c3scnc3C)cc2)C(C)C)[nH]n1]

In some embodiments, the degradation moiety is

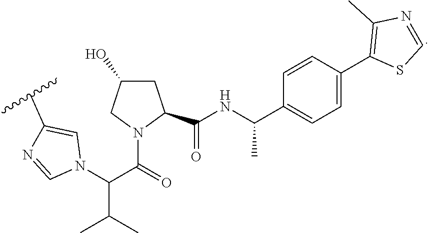 [SMILES: *c1cn(C(C(=O)N2C[C@H](O)C[C@H]2C(=O)N[C@@H](C)c2ccc(-c3scnc3C)cc2)C(C)C)cn1]

In some embodiments, the degradation moiety is

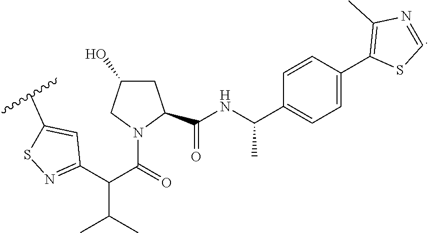 [SMILES: *c1cc(C(C(=O)N2C[C@H](O)C[C@H]2C(=O)N[C@@H](C)c2ccc(-c3scnc3C)cc2)C(C)C)ns1]

In some embodiments, the degradation moiety is

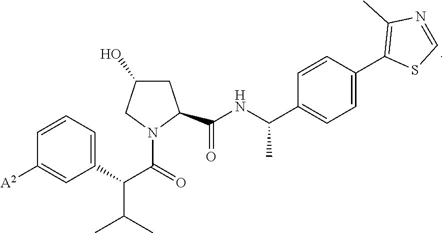

In some embodiments, the degradation moiety is

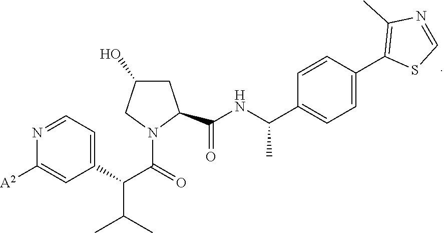

In some embodiments, the degradation moiety has the structure of Formula C5:

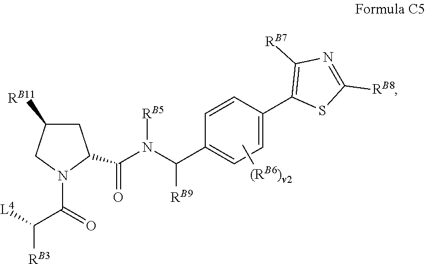

where
L⁴ is —N(R^{B1})(R^{B2}),

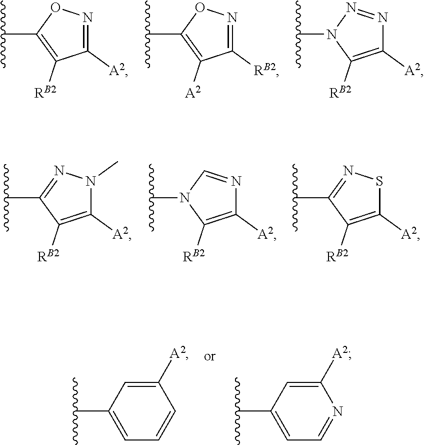

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₂-C₆ alkynyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl;
R^{B11} is H, alcohol, boronic acid, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
and
A² is a bond between the degradation moiety and the linker;
where one and only one of R^{B1}, R^{B3}, and R^{B6} is A²,
or a pharmaceutically acceptable salt thereof.

In some embodiments, R^{B11} is boric acid.

In some embodiments, the degradation moiety has the structure of Formula C6.

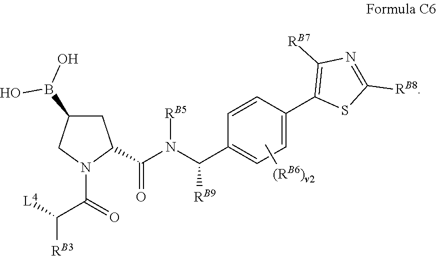

In some embodiments, the degradation moiety has the structure of Formula C1:

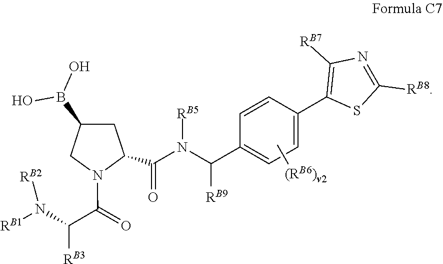

In some embodiments, the degradation moiety has the structure of Formula C8:

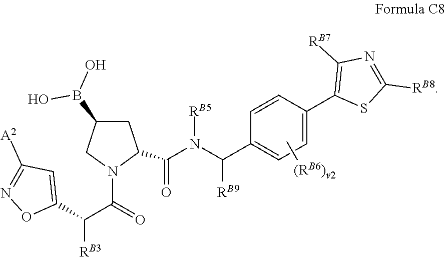

In some embodiments, R^{B9} is optionally substituted C₁-C₆ alkyl. In some embodiments, R^{B9} is methyl.

In some embodiments, R^{B9} is bonded to (S)-stereogenic center.

In some embodiments, v2 is 0. In some embodiments, R^{B5} is H. In some embodiments, R^{B7} is optionally substituted C₁-C₆ alkyl. In some embodiments, R^{B7} is methyl. In some embodiments, R^{B3} is optionally substituted C₁-C₆ alkyl. In some embodiments, R^{B3} is isopropyl. In some embodiments, R^{B8} is H. In some embodiments, R^{B2} is H.

In some embodiments, the degradation moiety is

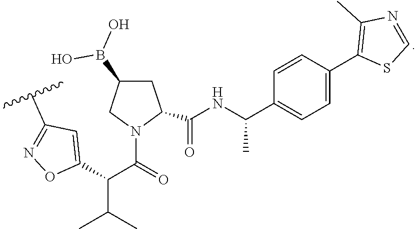 [SMILES: *c1cc([C@H](C(=O)N2C[C@@H](B(O)O)C[C@@H]2C(=O)N[C@@H](C)c2ccc(-c3scnc3C)cc2)C(C)C)on1]

In some embodiments, the degradation moiety has the structure of Formula D:

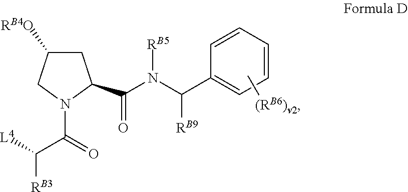

where
L⁴ is —N(R^{B1})(R^{B2}),

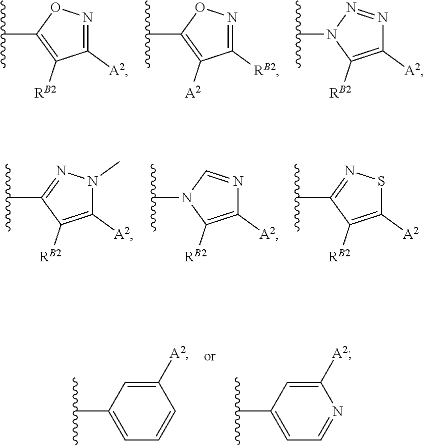

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₂-C₆ alkynyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
R^{B9} is H or optionally substituted C₁-C₆ alkyl; and
A² is a bond between the degradation moiety and the linker;
where one and only one of R^{B1}, R^{B3}, and R^{B6} is A²,
or a pharmaceutically acceptable salt thereof.

In some embodiments, the degradation moiety has the structure of Formula D3.

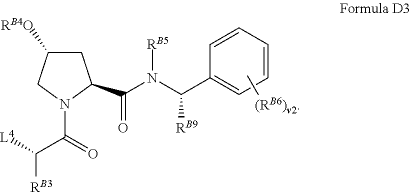

In some embodiments, the degradation moiety has the structure of Formula D1:

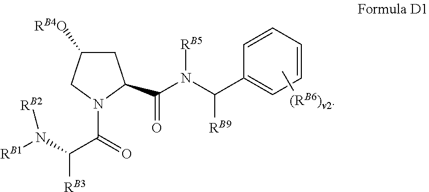

In some embodiments, the degradation moiety is

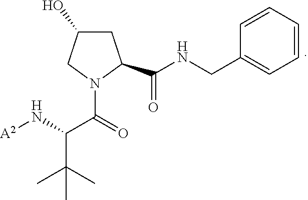

In some embodiments, the degradation moiety is

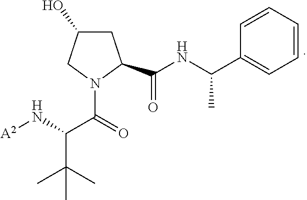

In some embodiments, the degradation moiety is

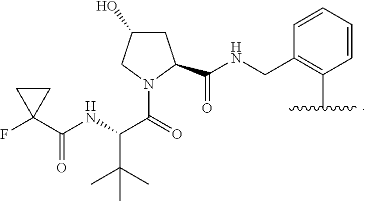 [SMILES: *c1ccccc1CNC(=O)[C@@H]1C[C@@H](O)CN1C(=O)[C@@H](NC(=O)C1(F)CC1)C(C)(C)C]

In some embodiments, the degradation moiety has the structure of Formula D2:

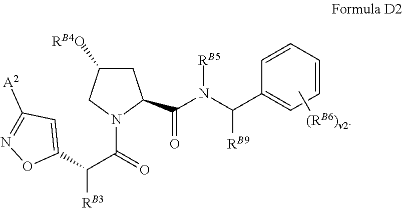

In some embodiments, R^{B9} is optionally substituted C₁-C₆ alkyl. In some embodiments, R^{B9} is methyl.

In some embodiments, R^{B9} is bonded to (S)-stereogenic center. In some embodiments, R^{B9} is H.

In some embodiments, v2 is 0. In some embodiments, v2 is 1. In some embodiments, v2 is 2. In some embodiments, R^{B4} is H. In some embodiments, R^{B5} is H. In some embodiments, R^{B3} is optionally substituted C₁-C₆ alkyl. In some embodiments, R^{B3} is isopropyl. In some embodiments, R^{B6} is H. In some embodiments, R^{B6} is halogen. In some embodiments, R^{B6} is fluorine. In some embodiments, R^{B6} is bromine. In some embodiments, R^{B6} is chlorine. In some embodiments, R^{B6} is cyano. In some embodiments, R^{B6} is optionally substituted C₁-C₆ heteroalkyl. In some embodiments, R^{B6} is optionally substituted C₃-C₆ alkynyl. In some embodiments, R^{B6} is methoxy. In some embodiments, R^{B6} is 3-methoxy-1-propanoxy.

In some embodiments, the degradation moiety is

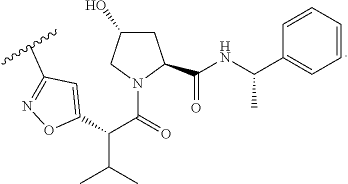 [SMILES: *c1cc([C@H](C(=O)N2C[C@H](O)C[C@H]2C(=O)N[C@@H](C)c2ccccc2)C(C)C)on1]

In some embodiments, the degradation moiety is

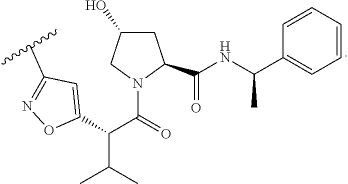 [SMILES: *c1cc([C@H](C(=O)N2C[C@H](O)C[C@H]2C(=O)N[C@H](C)c2ccccc2)C(C)C)on1]

In some embodiments, the degradation moiety is

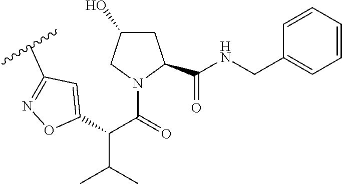 [SMILES: *c1cc([C@H](C(=O)N2C[C@H](O)C[C@H]2C(=O)NCc2ccccc2)C(C)C)on1]

In some embodiments, the degradation moiety is

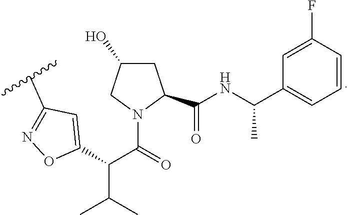 [SMILES: *c1cc([C@H](C(=O)N2C[C@H](O)C[C@H]2C(=O)N[C@@H](C)c2cccc(F)c2)C(C)C)on1]

In some embodiments, the degradation moiety is

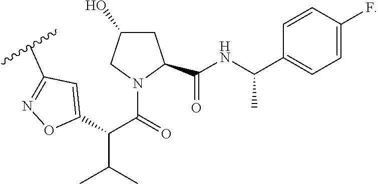 [SMILES: *c1cc([C@H](C(=O)N2C[C@H](O)C[C@H]2C(=O)N[C@@H](C)c2ccc(F)cc2)C(C)C)on1]

In some embodiments, the degradation moiety is

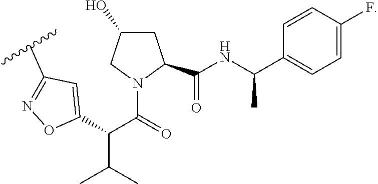 [SMILES: *c1cc([C@H](C(=O)N2C[C@H](O)C[C@H]2C(=O)N[C@H](C)c2ccc(F)cc2)C(C)C)on1]

In some embodiments, the degradation moiety is

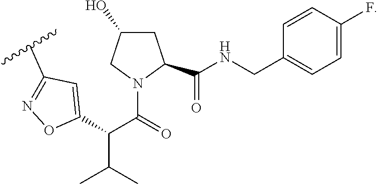 [SMILES: *c1cc([C@H](C(=O)N2C[C@H](O)C[C@H]2C(=O)NCc2ccc(F)cc2)C(C)C)on1]

In some embodiments, the degradation moiety is

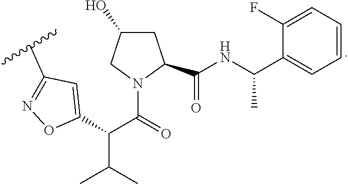 [SMILES: *c1cc([C@H](C(=O)N2C[C@H](O)C[C@H]2C(=O)N[C@@H](C)c2ccccc2F)C(C)C)on1]

In some embodiments, the degradation moiety is

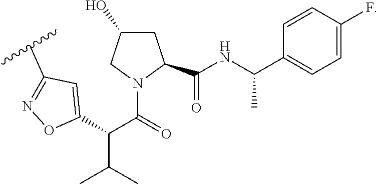 [SMILES: *c1cc([C@H](C(=O)N2C[C@H](O)C[C@H]2C(=O)N[C@@H](C)c2ccc(F)cc2)C(C)C)on1]

In some embodiments, the degradation moiety is

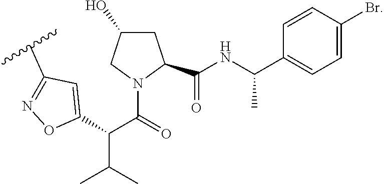 [SMILES: *c1cc([C@H](C(=O)N2C[C@H](O)C[C@H]2C(=O)N[C@@H](C)c2ccc(Br)cc2)C(C)C)on1]

In some embodiments, the degradation moiety is

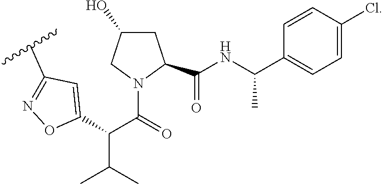 [SMILES: *c1cc([C@H](C(=O)N2C[C@H](O)C[C@H]2C(=O)N[C@@H](C)c2ccc(Cl)cc2)C(C)C)on1]

In some embodiments, the degradation moiety is

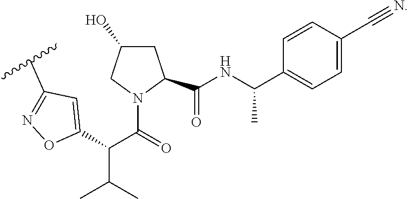 [SMILES: *c1cc([C@H](C(=O)N2C[C@H](O)C[C@H]2C(=O)N[C@@H](C)c2ccc(C#N)cc2)C(C)C)on1]

In some embodiments, the degradation moiety is

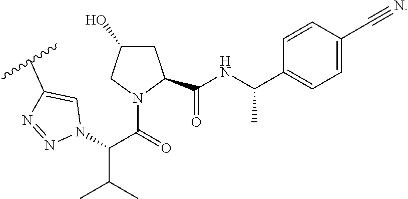 [SMILES: *c1cn([C@H](C(=O)N2C[C@H](O)C[C@H]2C(=O)N[C@@H](C)c2ccc(C#N)cc2)C(C)C)nn1]

In some embodiments, the degradation moiety is

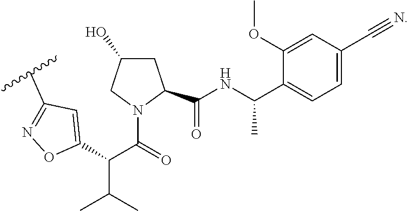 [SMILES: *c1cc([C@H](C(=O)N2C[C@H](O)C[C@H]2C(=O)N[C@@H](C)c2ccc(C#N)cc2OC)C(C)C)on1]

In some embodiments, the degradation moiety is

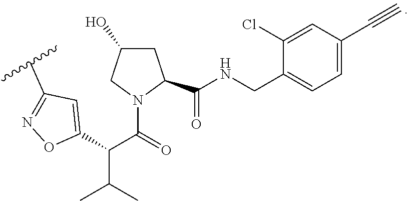 [SMILES: *c1cc([C@H](C(=O)N2C[C@H](O)C[C@H]2C(=O)NCc2ccc(C#C)cc2Cl)C(C)C)on1]

In some embodiments, the degradation moiety is

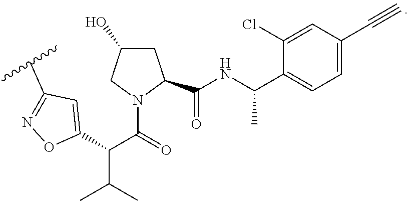 [SMILES: *c1cc([C@H](C(=O)N2C[C@H](O)C[C@H]2C(=O)N[C@@H](C)c2ccc(C#C)cc2Cl)C(C)C)on1]

In some embodiments, the degradation moiety is

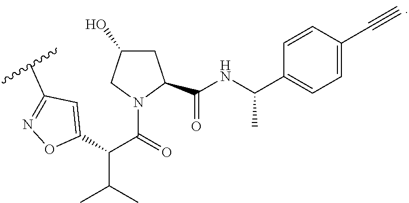 [SMILES: *c1cc([C@H](C(=O)N2C[C@H](O)C[C@H]2C(=O)N[C@@H](C)c2ccc(C#C)cc2)C(C)C)on1]

In some embodiments, the degradation moiety is

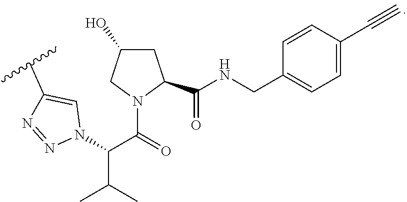 [SMILES: *c1cn([C@H](C(=O)N2C[C@H](O)C[C@H]2C(=O)NCc2ccc(C#C)cc2)C(C)C)nn1]

In some embodiments, the degradation moiety is

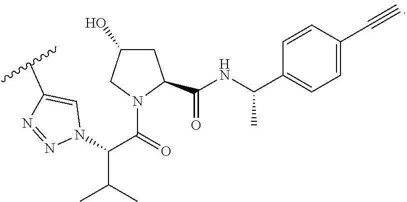 [SMILES: *c1cn([C@H](C(=O)N2C[C@H](O)C[C@H]2C(=O)N[C@@H](C)c2ccc(C#C)cc2)C(C)C)nn1]

In some embodiments, the degradation moiety is

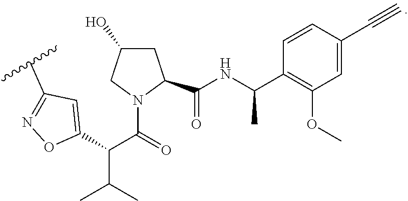 [SMILES: *c1cc([C@H](C(=O)N2C[C@H](O)C[C@H]2C(=O)N[C@H](C)c2ccc(C#C)cc2OC)C(C)C)on1]

In some embodiments, the degradation moiety is

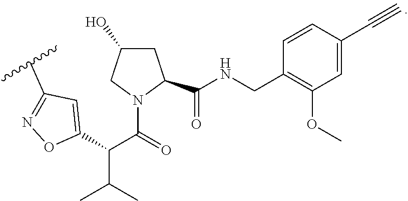 [SMILES: *c1cc([C@H](C(=O)N2C[C@H](O)C[C@H]2C(=O)NCc2ccc(C#C)cc2OC)C(C)C)on1]

In some embodiments, the degradation moiety is

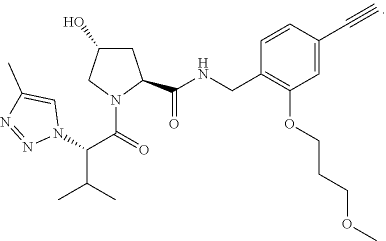 [SMILES: C#Cc1ccc(CNC(=O)[C@@H]2C[C@@H](O)CN2C(=O)[C@H](C(C)C)n2cc(C)nn2)c(OCCCOC)c1]

In some embodiments, the degradation moiety is

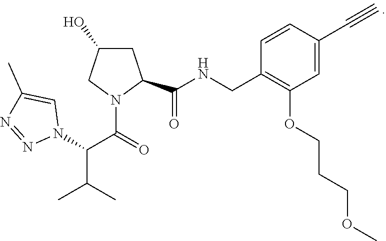 [SMILES: C#Cc1ccc(CNC(=O)[C@@H]2C[C@@H](O)CN2C(=O)[C@H](C(C)C)n2cc(C)nn2)c(OCCCOC)c1]

In some embodiments, the degradation moiety is

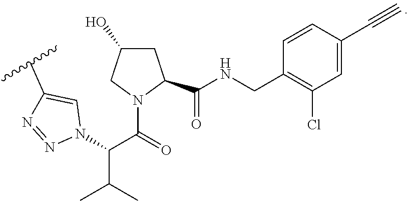 [SMILES: *c1cn([C@H](C(=O)N2C[C@H](O)C[C@H]2C(=O)NCc2ccc(C#C)cc2Cl)C(C)C)nn1]

In some embodiments, the degradation moiety is

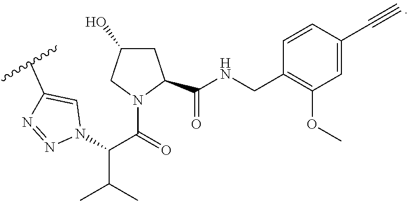 [SMILES: *c1cn([C@H](C(=O)N2C[C@H](O)C[C@H]2C(=O)NCc2ccc(C#C)cc2OC)C(C)C)nn1]

In some embodiments, the degradation moiety has the structure of Formula Da:

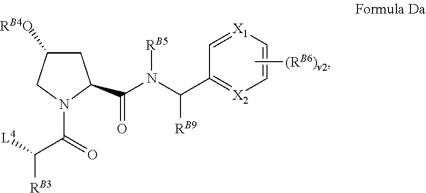

where
L⁴ is —N(R^{B1})(R^{B2}),

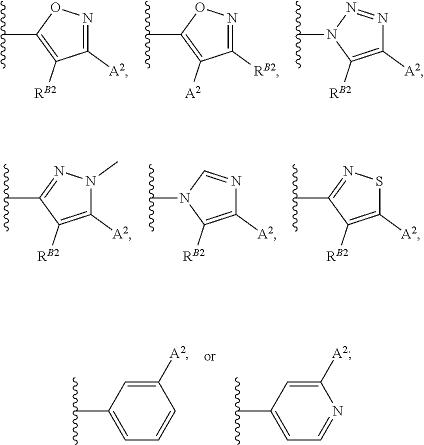

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
Each of X₁ and X₂ are, independently, C, N, or O.
v2 is 0, 1, 2, 3, or 4;
each R^{B6} is, independently, A², halogen, optionally substituted C₁-C₆ alkyl, optionally substituted C₂-C₆ alkynyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
R^{B9} is H or optionally substituted C₁-C₆ alkyl; and
A² is a bond between the degradation moiety and the linker;
where one and only one of R^{B1}, R^{B3}, and R^{B6} is A²,
or a pharmaceutically acceptable salt thereof.

In some embodiments, the degradation moiety has the structure of Formula Da3.

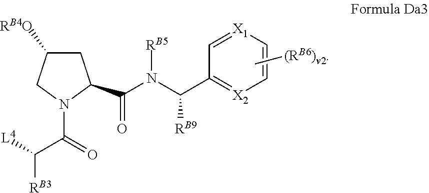

In some embodiments, the degradation moiety has the structure of Formula Da1:

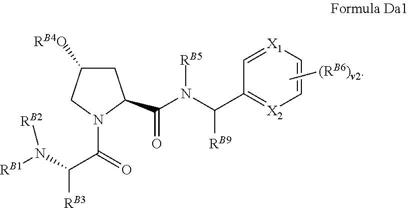

In some embodiments, the degradation moiety has the structure of Formula Da2:

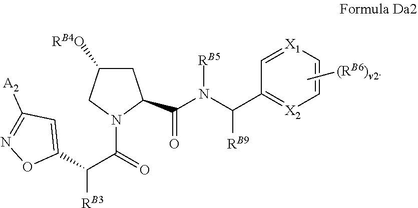

In some embodiments, R^{B9} is optionally substituted C₁-C₆ alkyl. In some embodiments, R^{B9} is methyl.

In some embodiments, R^{B9} is bonded to (S)-stereogenic center.

In some embodiments, v2 is 0. In some embodiments, R^{B4} is H. In some embodiments, R^{B5} is H. In some embodiments, R^{B3} is optionally substituted C₁-C₆ alkyl. In some embodiments, R^{B3} is isopropyl. In some embodiments, R^{B2} is H. In some embodiments, X₁ is C. In some embodiments, X₂ is N.

In some embodiments, the degradation moiety is

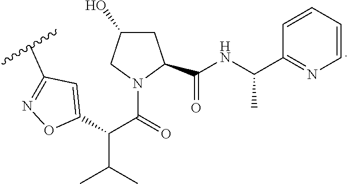 [SMILES: *c1cc([C@H](C(=O)N2C[C@H](O)C[C@H]2C(=O)N[C@@H](C)c2ccccn2)C(C)C)on1]

In some embodiments, the degradation moiety has the structure of Formula E:

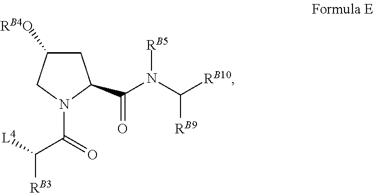

where
L⁴ is —N(R^{B1})(R^{B2}),

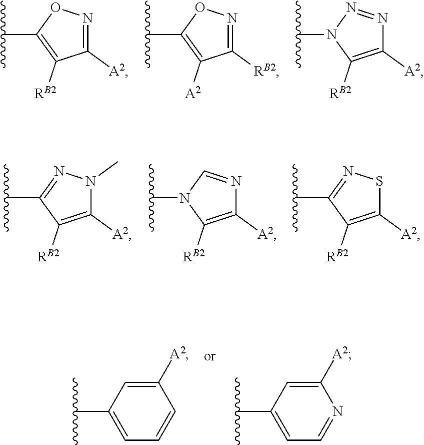

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
R^{B9} is H, optionally substituted C₁-C₆ alkyl, optionally substituted C₃-C₆ alkynyl, optionally substituted C₃-C₁₀ carbocyclyl, or optionally substituted C₂-C₁₀ heterocyclyl;
B¹⁰ is, H, optionally substituted C₁-C₆ alkyl, optionally substituted C₃-C₆ alkynyl, optionally substituted C₃-C₁₀ carbocyclyl, optionally substituted C₂-C₁₀ heterocyclyl; optionally substituted amino, or cyano, and
A² is a bond between the degradation moiety and the linker;
where one and only one of R^{B1}, R^{B3}, and R^{B6} is A²,
or a pharmaceutically acceptable salt thereof.

In some embodiments, the degradation moiety has the structure of Formula E3.

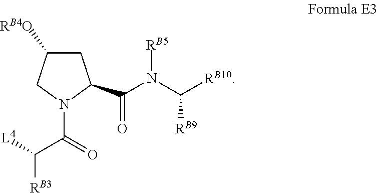

In some embodiments, the degradation moiety has the structure of Formula E1:

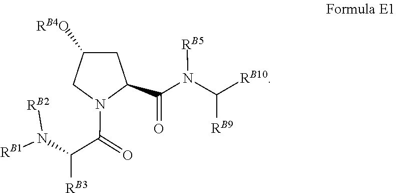

In some embodiments, the degradation moiety is

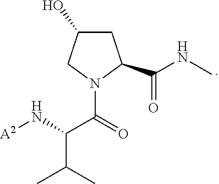

In some embodiments, the degradation moiety is

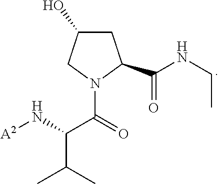

In some embodiments, the degradation moiety has the structure of Formula E2:

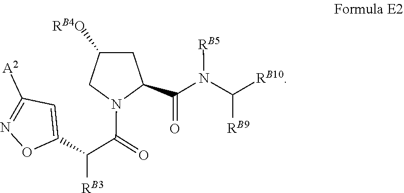

In some embodiments, R^{B9} is optionally substituted C₁-C₆ alkyl. In some embodiments, R^{B9} is methyl.

In some embodiments, R^{B9} is bonded to (S)-stereogenic center.

In some embodiments, v2 is 0. In some embodiments, v2 is 1. In some embodiments, R^{B4} is H. In some embodiments, R^{B5} is H. In some embodiments, R^{B3} is optionally substituted C₁-C₆ alkyl. In some embodiments, R^{B3} is isopropyl. In some embodiments, R^{B2} is H. In some embodiments, R^{B9} is optionally substituted C₁-C₆ alkyl. In some embodiments, R^{B9} is methyl. In some embodiments, R^{B9} is H. In some embodiments, R^{B9} is optionally substituted C₃-C₆ alkynyl. In some embodiments, R^{B10} is absent. In some embodiments, R^{B9} is [1.1.1]pentane. In some embodiments, R^{B9} is cyclopropane. In some embodiments, R^{B9} is cyclobutane. In some embodiments, R^{B9} is cyclopentane. In some embodiments, R^{B10} is H. In some embodiments, R^{B10} is cyano. In some embodiments, R^{B10} is optionally substituted C₃-C₁₀ carbocyclyl, In some embodiments, R^{B10} is optionally substituted C₁-C₆ alkyl. In some embodiments, R^{B10} is methyl.

In some embodiments, the degradation moiety is

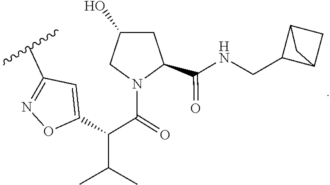 [SMILES: *c1cc([C@H](C(=O)N2C[C@H](O)C[C@H]2C(=O)NCC2C3CC2C3)C(C)C)on1]

In some embodiments, the degradation moiety is

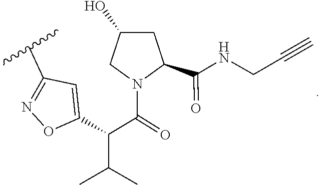 [SMILES: *c1cc([C@H](C(=O)N2C[C@H](O)C[C@H]2C(=O)NCC#C)C(C)C)on1]

In some embodiments, the degradation moiety is

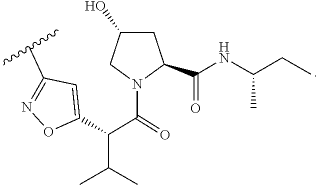 [SMILES: *c1cc([C@H](C(=O)N2C[C@H](O)C[C@H]2C(=O)N[C@@H](C)CC)C(C)C)on1]

In some embodiments, the degradation moiety is

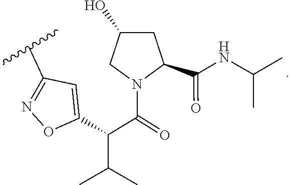 [SMILES: *c1cc([C@H](C(=O)N2C[C@H](O)C[C@H]2C(=O)NC(C)C)C(C)C)on1]

In some embodiments, the degradation moiety is

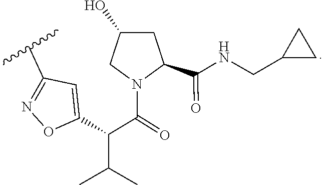 [SMILES: *c1cc([C@H](C(=O)N2C[C@H](O)C[C@H]2C(=O)NCC2CC2)C(C)C)on1]

In some embodiments, the degradation moiety is

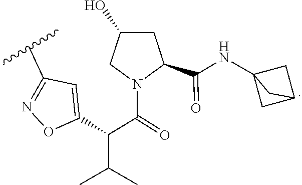 [SMILES: *c1cc([C@H](C(=O)N2C[C@H](O)C[C@H]2C(=O)NC23CC(C2)C3)C(C)C)on1]

In some embodiments, the degradation moiety is

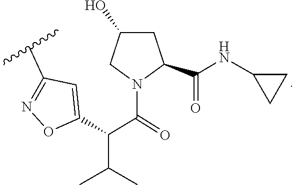 [SMILES: *c1cc([C@H](C(=O)N2C[C@H](O)C[C@H]2C(=O)NC2CC2)C(C)C)on1]

In some embodiments, the degradation moiety is

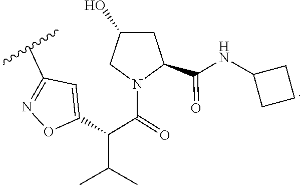 [SMILES: *c1cc([C@H](C(=O)N2C[C@H](O)C[C@H]2C(=O)NC2CCC2)C(C)C)on1]

In some embodiments, the degradation moiety is

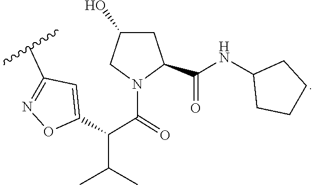 [SMILES: *c1cc([C@H](C(=O)N2C[C@H](O)C[C@H]2C(=O)NC2CCCC2)C(C)C)on1]

In some embodiments, the degradation moiety has the structure of Formula F:

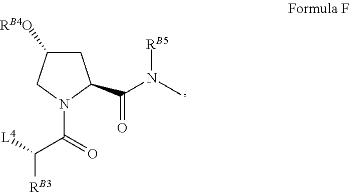

where
L⁴ is —N(R^{B1})(R^{B2}),

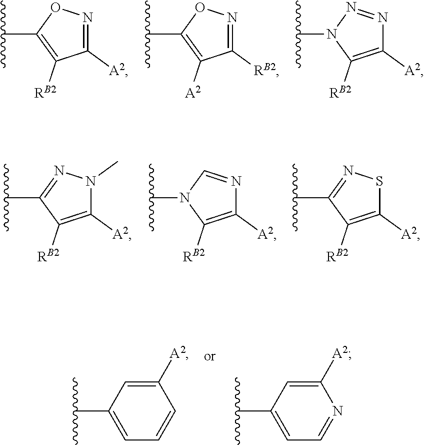

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
A² is a bond between the degradation moiety and the linker;
where one and only one of R^{B1} or R^{B3} is A²,
or a pharmaceutically acceptable salt thereof.

In some embodiments, the degradation moiety has the structure of Formula F3.

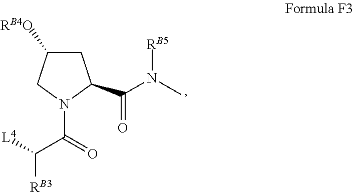

In some embodiments, the degradation moiety has the structure of Formula F1:

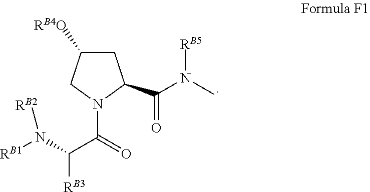

In some embodiments, the degradation moiety is

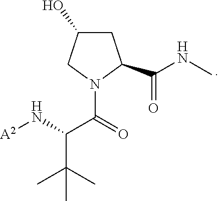

In some embodiments, the degradation moiety is

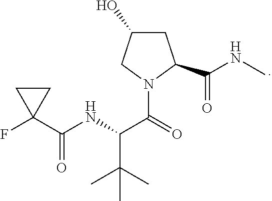 [SMILES: CNC(=O)[C@@H]1C[C@@H](O)CN1C(=O)[C@@H](NC(=O)C1(F)CC1)C(C)(C)C]

In some embodiments, the degradation moiety has the structure of Formula F2:

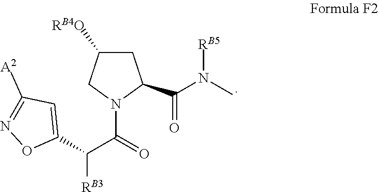

In some embodiments, R^{B9} is optionally substituted C₁-C₆ alkyl. In some embodiments, R^{B9} is methyl.

In some embodiments, R^{B4} is H. In some embodiments, R^{B5} is H. In some embodiments, R^{B3} is optionally substituted C₁-C₆ alkyl. In some embodiments, R^{B3} is isopropyl. In some embodiments, R^{B2} is H.

In some embodiments, the degradation moiety is

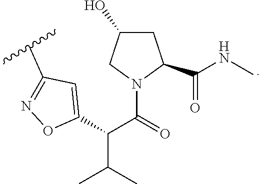 [SMILES: *c1cc([C@H](C(=O)N2C[C@H](O)C[C@H]2C(=O)NC)C(C)C)on1]

In some embodiments, the linker has the structure of Formula II:

A¹-(B¹)_{f}-(C¹)_{g}-(B²)ₕ-(D)-(B³)ᵢ-(C²)ⱼ-(B⁴)ₖ-A², Formula II
or a pharmaceutically acceptable salt thereof,
where
A¹ is a bond between the linker and ring system A;
A² is a bond between the degradation moiety and the linker;
each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₃-C₁₀ carbocyclyl, optionally substituted C₂-C₈ heterocyclyl, optionally substituted C₂-C₆ heteroaryl, optionally substituted C₆₋₁₂ aryl, O, S, S(O)₂, or NRN;
each R^{N} is, independently, H, optionally substituted C₁₋₄ alkyl, optionally substituted C₂₋₄ alkenyl, optionally substituted C₂₋₄ alkynyl, optionally substituted C₂₋₁₀ heterocyclyl, optionally substituted C₂₋₆ heteroaryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₁₀ heterocyclyl, optionally substituted C₂₋₆ heteroaryl, optionally substituted C₆₋₁₂ aryl, optionally substituted C₂-C₁₀ polyethylene glycol, optionally substituted C₃-C₁₀ cycloalkyl, optionally substituted C₃-C₁₀ carbocyclyl, or optionally substituted C₁₋₁₀ heteroalkyl; or D is absent, and the linker is A¹-(B¹)_{f}-(C¹)_{g}-(B²)ₕ-(B³)ᵢ-(C²)ⱼ-(B⁴)ₖ-A².

In some embodiments, each of B¹, B², B³, and B⁴ is, independently, optionally substituted C₁-C₂ alkyl, optionally substituted C₁-C₃ heteroalkyl, optionally substituted C₂-C₁₀ heterocyclyl, optionally substituted C₂₋₆ heteroaryl, O, or NRN; and 0 is optionally substituted C₁₋₁₀ alkyl, optionally substituted C₂₋₁₀ alkenyl, optionally substituted C₂₋₁₀ alkynyl, optionally substituted C₂₋₁₀ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}-(C¹)_{g}-(B²)ₕ- to —(B³)ᵢ—(C²)ⱼ-(B⁴)ₖ-A².

In some embodiments, each of B¹, B², B³, and B⁴ is, independently, optionally substituted C₁-C₂ alkyl, optionally substituted C₁-C₃ heteroalkyl, optionally substituted C₂-C₁₀ heterocyclyl, optionally substituted C₂₋₆ heteroaryl, optionally substituted C₃-C₁₀ cycloalkyl, optionally substituted C₃-C₁₀ carbocyclyl, O, or NR^{N}.

In some embodiments, each of B¹ and B⁴ is, independently,

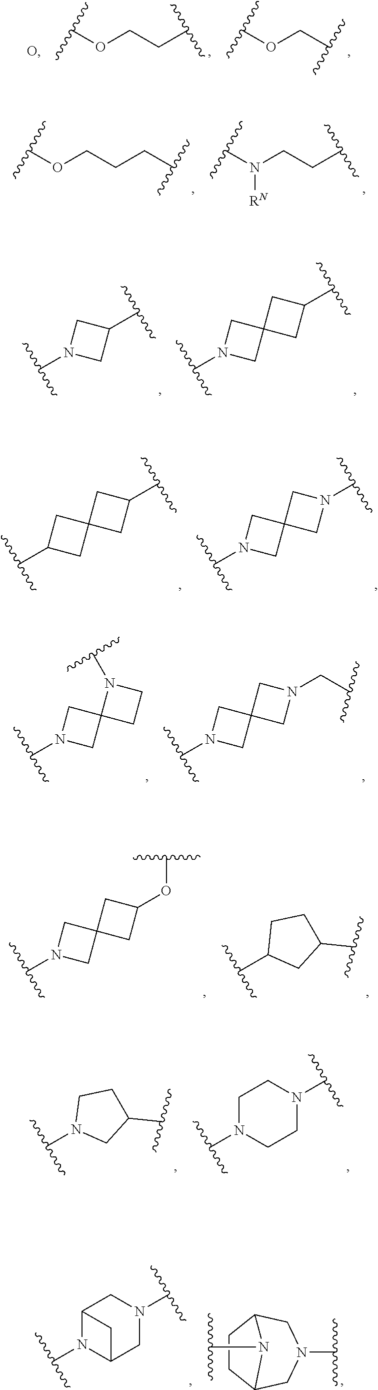

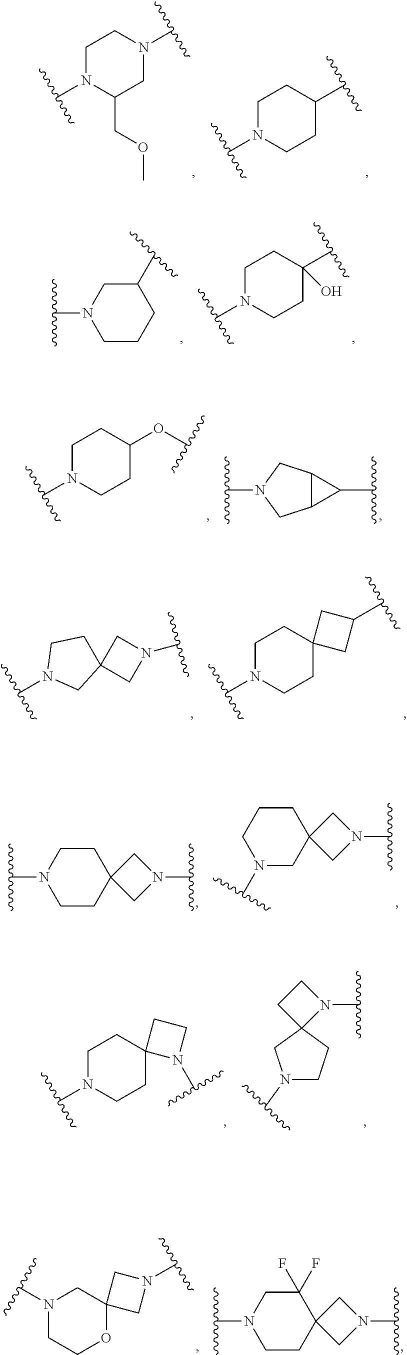

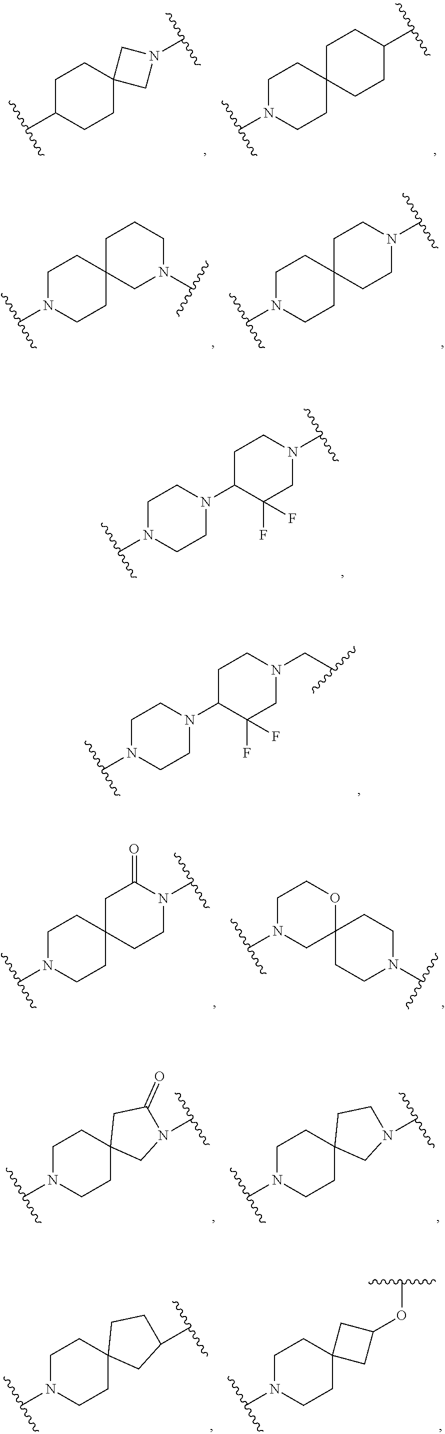

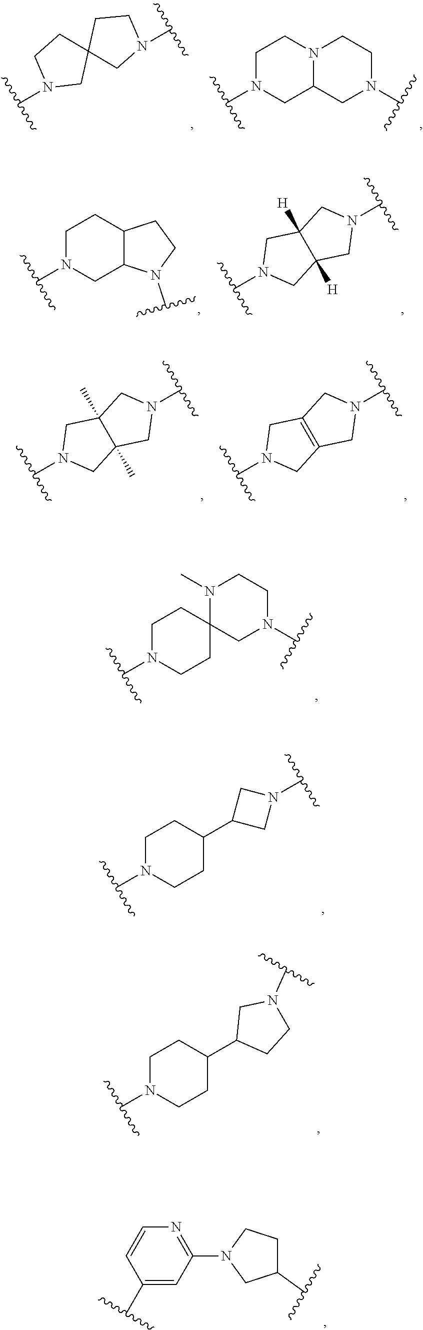

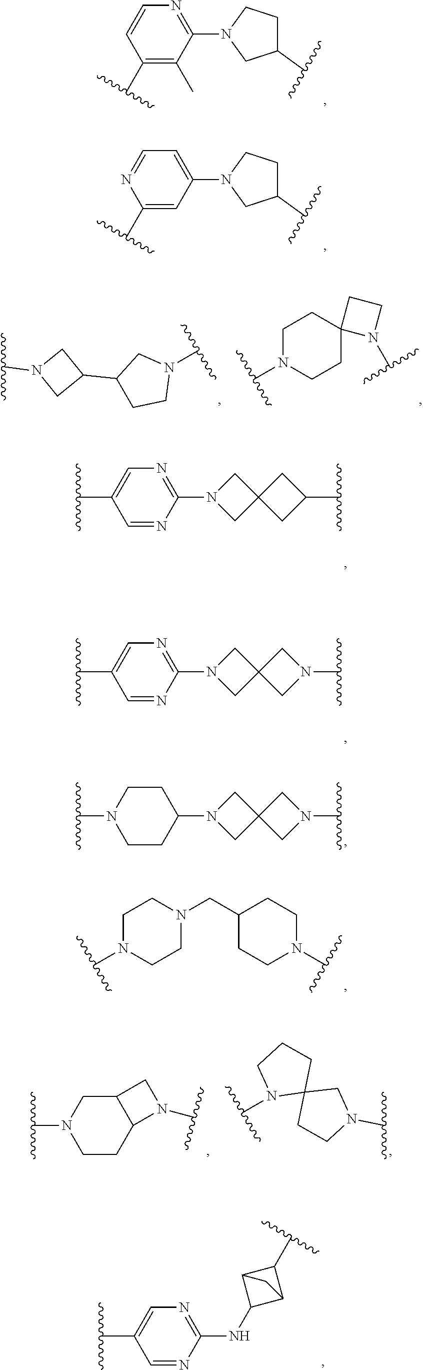

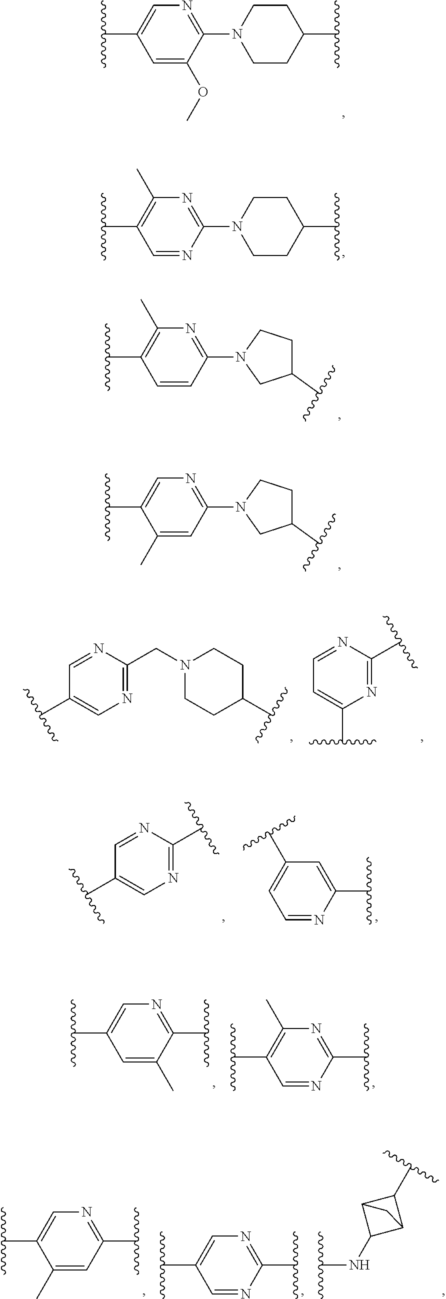

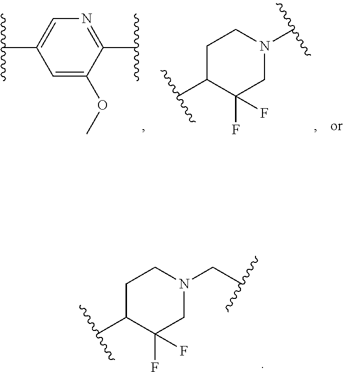

In some embodiments, B¹ is

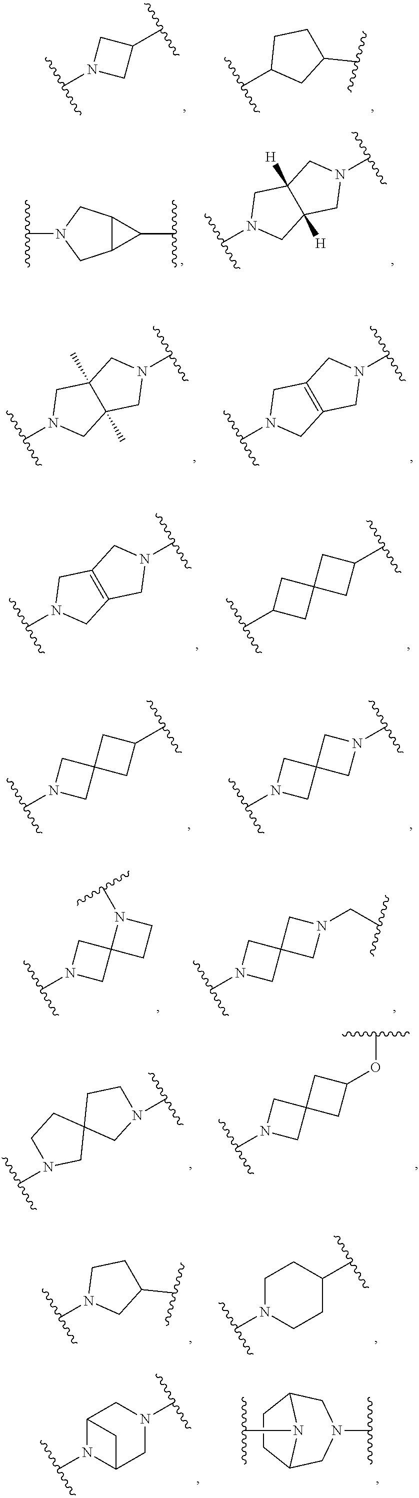

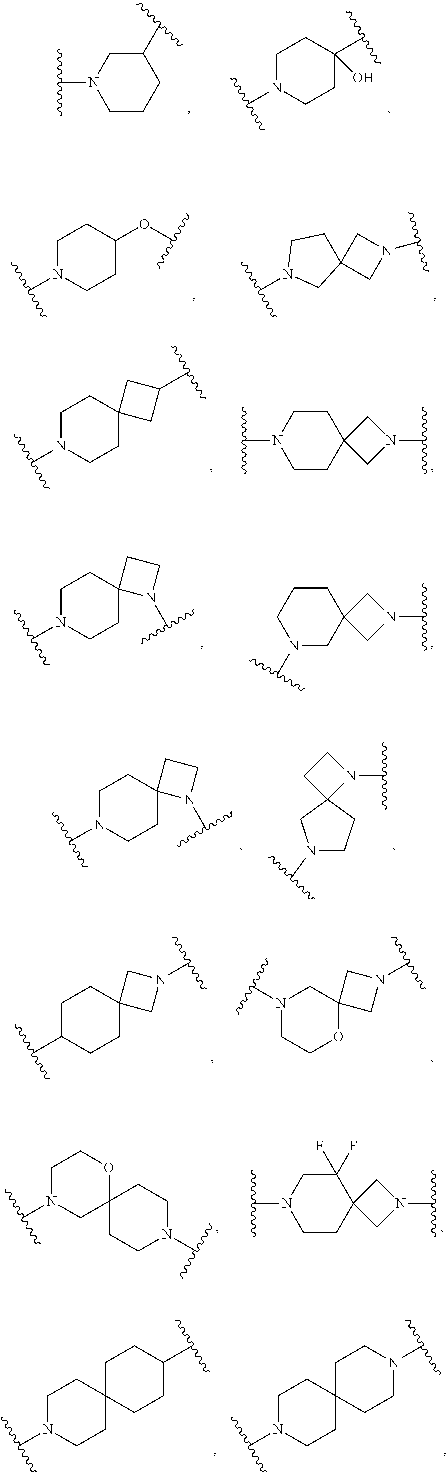

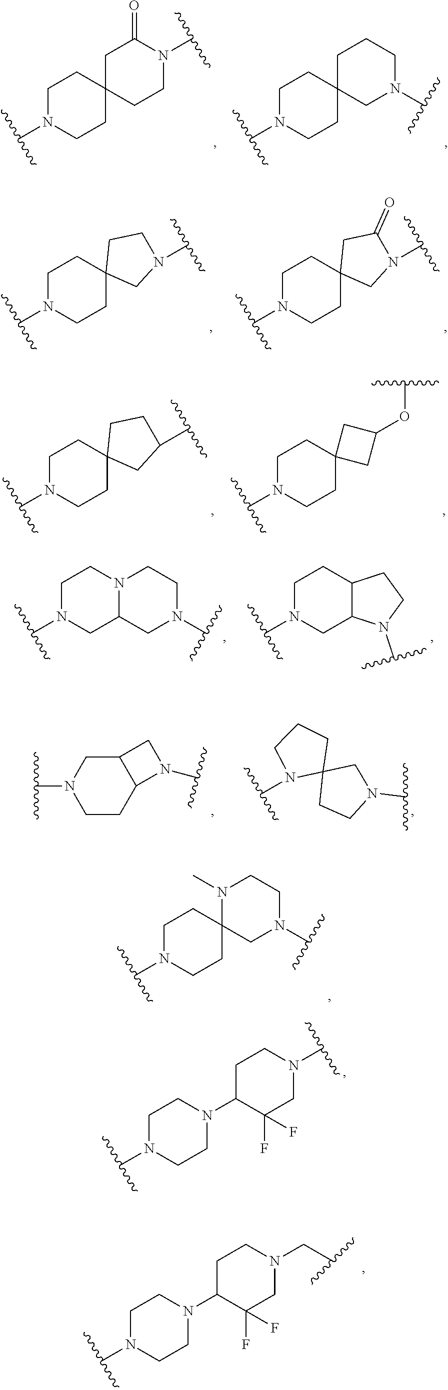

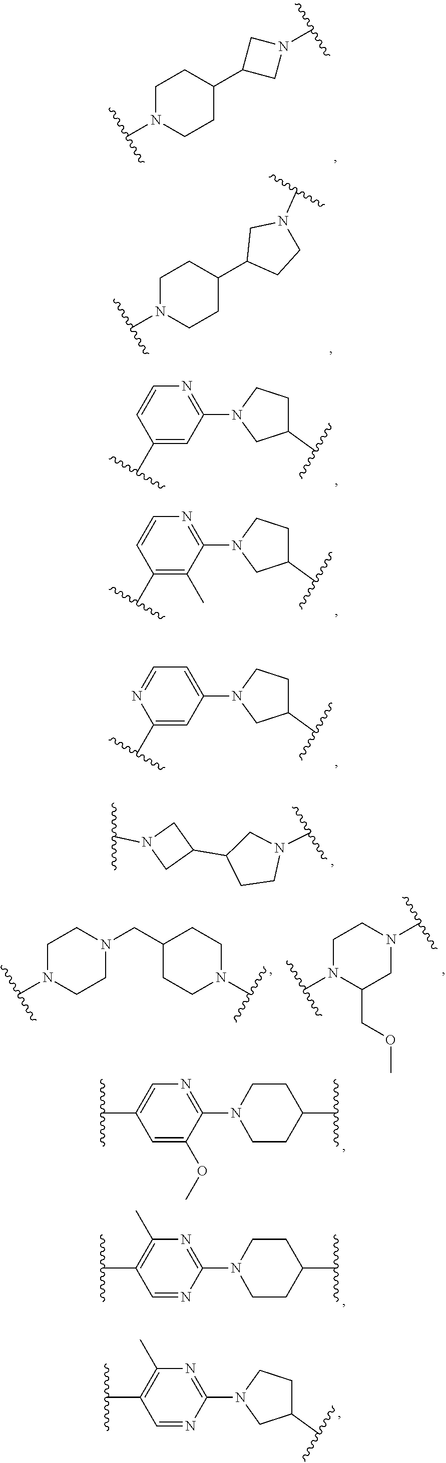

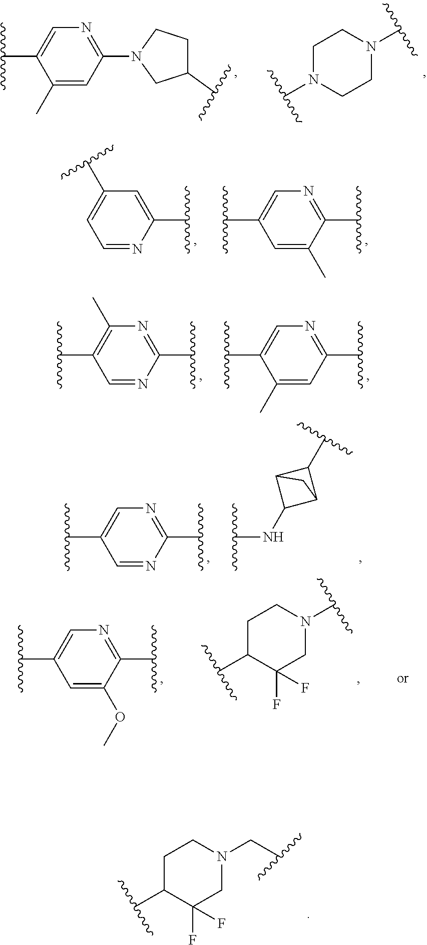

In some embodiments, B⁴ is

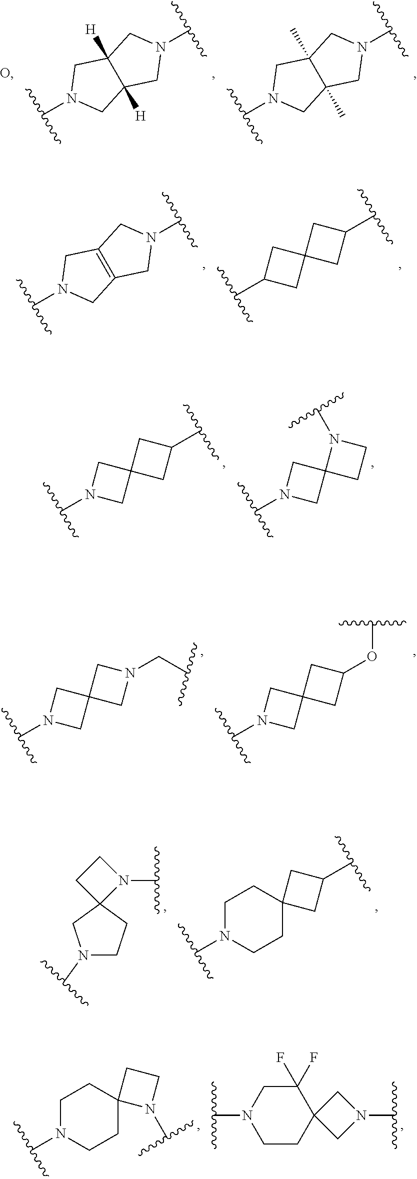

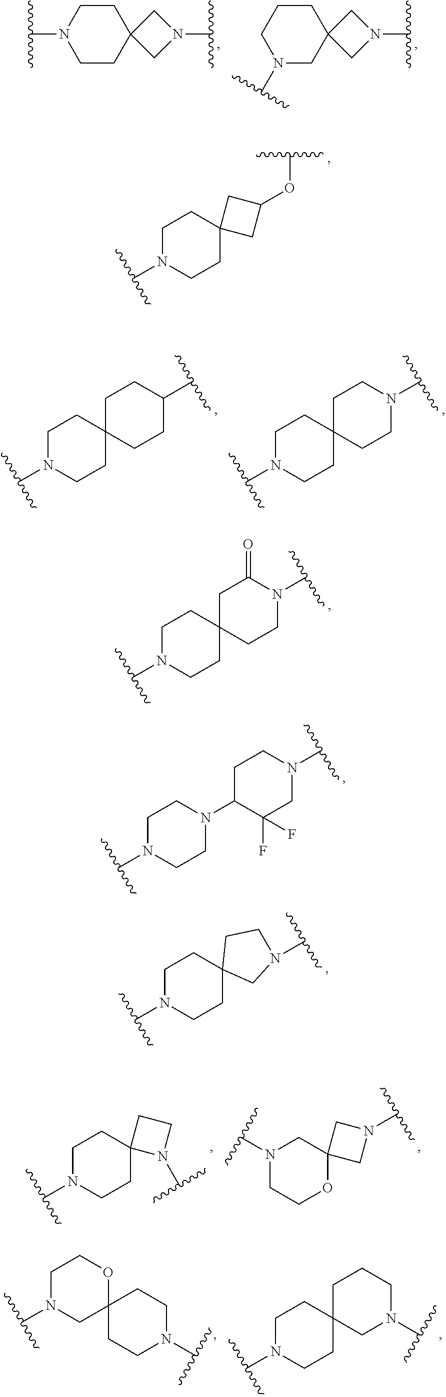

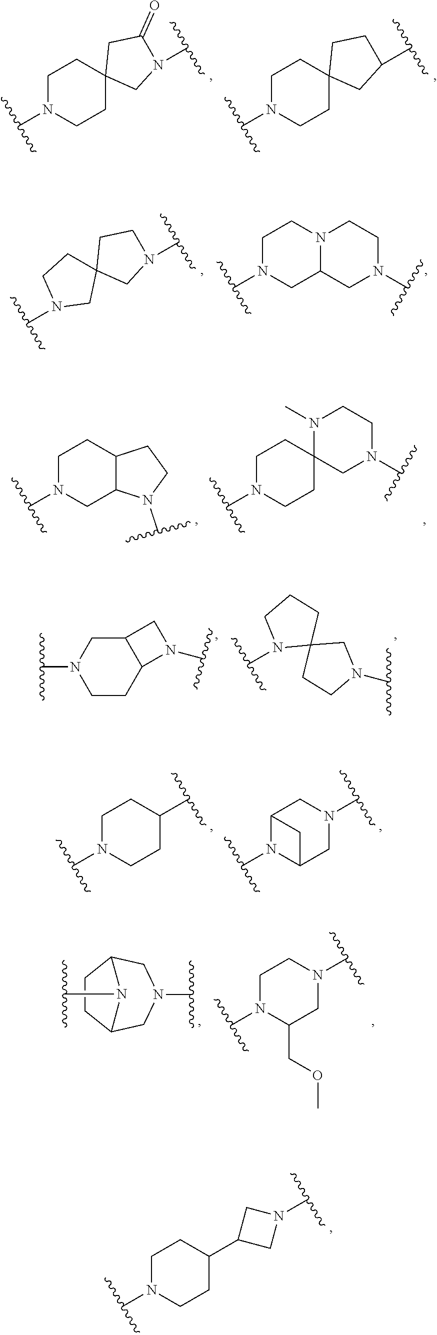

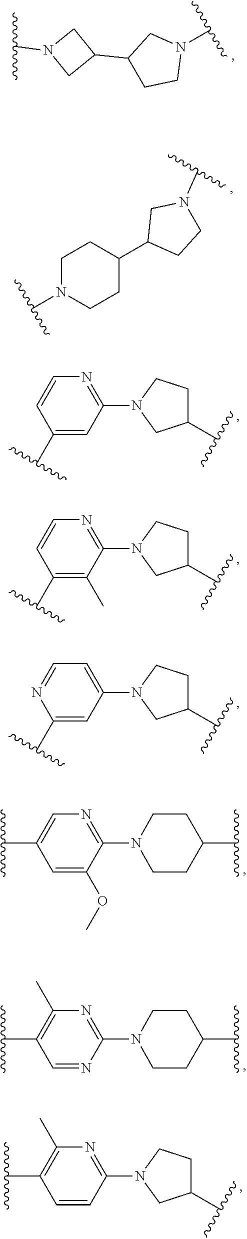

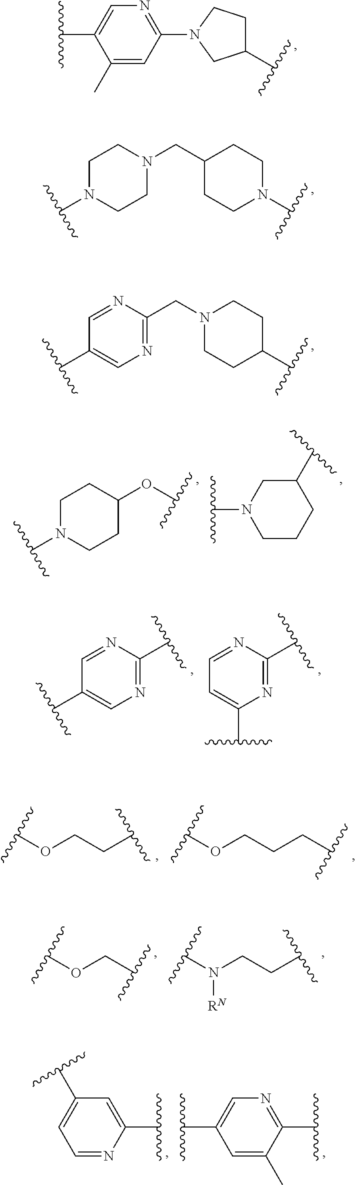

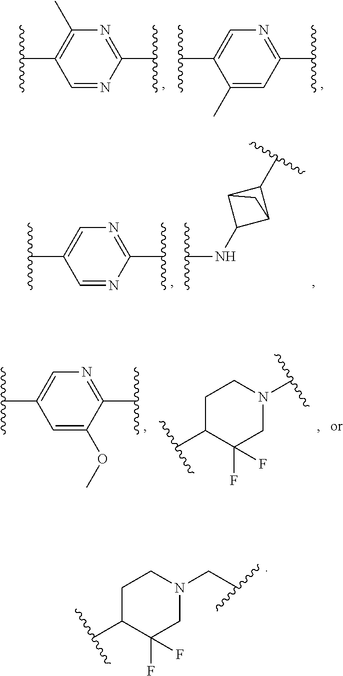

In some embodiments, C¹ is

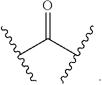 [SMILES: *C(*)=O]

In some embodiments, B² is optionally substituted C₁-C₄ alkyl.

In some embodiments, D is optionally substituted C₁-C₁₀ alkyl.

In some embodiments, f is 1. In some embodiments, g is 0. In some embodiments, g is 1. In some embodiments, h is 0. In some embodiments, h is 1. In some embodiments, i is 0. In some embodiments, i is 1. In some embodiments, j is 0. In some embodiments, j is 1. In some embodiments, k is 0. In some embodiments, k is 1.

In some embodiments, D is absent, and the linker is A¹-(B¹)_{f}-(C¹)_{g}-(B²)ₕ-(B³)ᵢ-(C²)ⱼ-(B⁴)ₖ-A².

In some embodiments, the linker is D. In some embodiments, D is optionally substituted C₁₋₁₀ alkyl, optionally substituted C₂₋₁₀ alkenyl, optionally substituted C₂₋₁₀ alkynyl, optionally substituted C₂₋₁₀ heterocyclyl, optionally substituted C₂₋₆ heteroaryl, optionally substituted C₆₋₁₂ aryl, optionally substituted C₂-C₁₀ polyethylene glycol, or optionally substituted C₁₋₁₀ heteroalkyl. In some embodiments, D is optionally substituted C₃-C₁₀ cycloalkyl, f is 1, g is 0, h is 0, i is 0, j is 0, and, k is 1. In some embodiments, D is optionally substituted C₃-C₁₀ cycloalkyl, f is 1, g is 0, h is 0, i is 0, j is 0, and, k is 0. In some embodiments, D is optionally substituted C₃-C₁₀ cycloalkyl, f is 0, g is 0, h is 0, i is 0, j is 0, and, k is 1. In some embodiments, D is optionally substituted C₃-C₁₀ cycloalkyl, f is 0, g is 0, h is 0, i is 0, j is 0, and, k is 0. In some embodiments, D is optionally substituted C₃-C₁₀ carbocyclyl, f is 1, g is 0, h is 0, i is 0, j is 0, and, k is 1. In some embodiments, D is optionally substituted C₃-C₁₀ carbocyclyl, f is 1, g is 0, h is 0, i is 0, j is 0, and, k is 0. In some embodiments, D is optionally substituted C₃-C₁ carbocyclyl, f is 0, g is 0, h is 0, i is 0, j is 0, and, k is 1. In some embodiments, D is optionally substituted C₃-C₁₀ carbocyclyl, f is 0, g is 0, h is 0, i is 0, j is 0, and, k is 0. In some embodiments, D is:

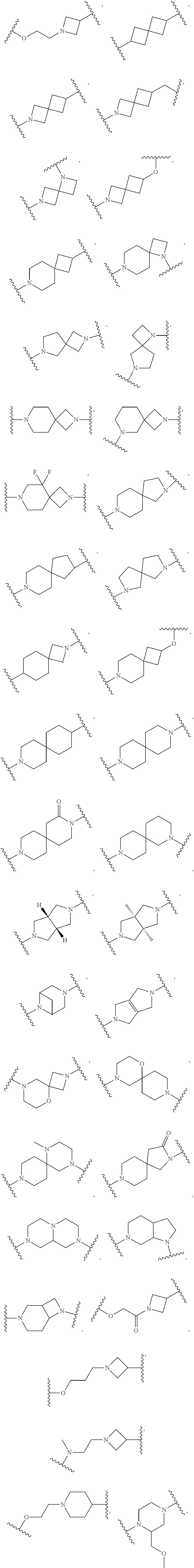

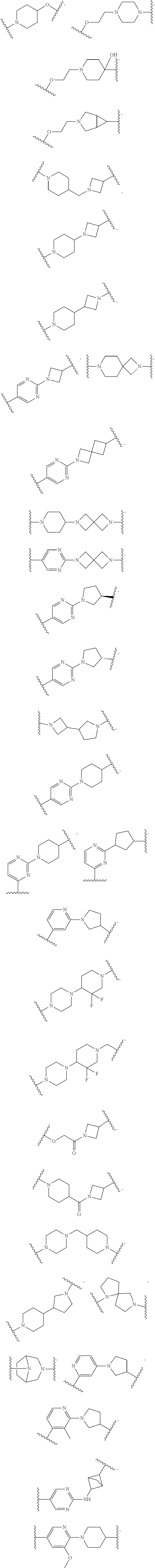

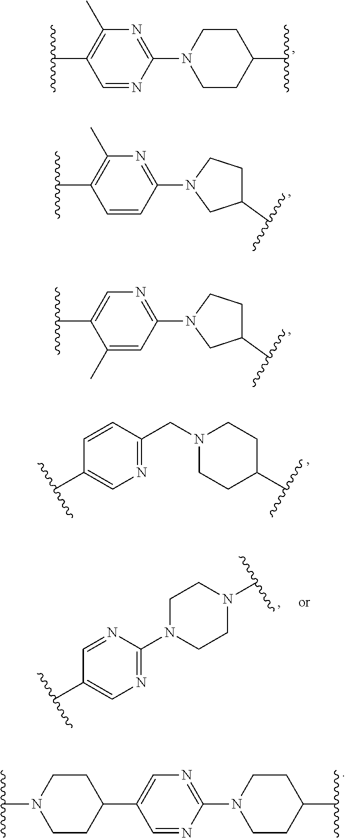

In some embodiments, the linker has the structure of

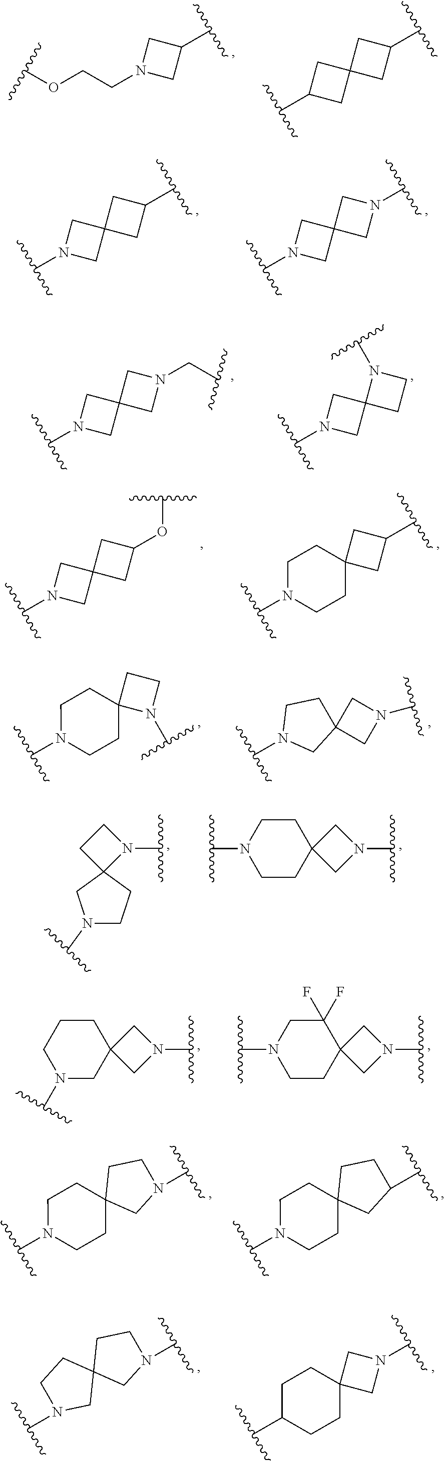

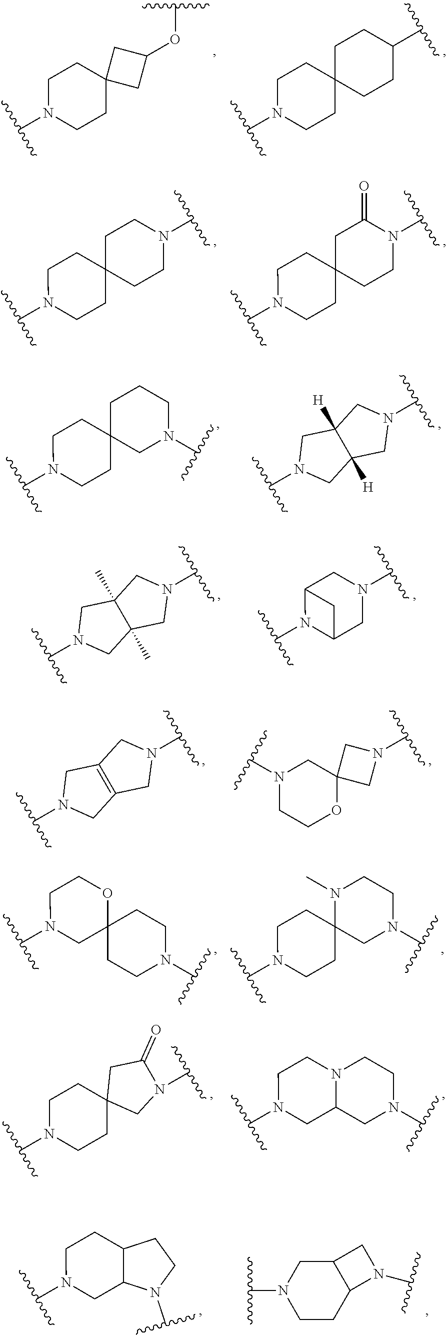

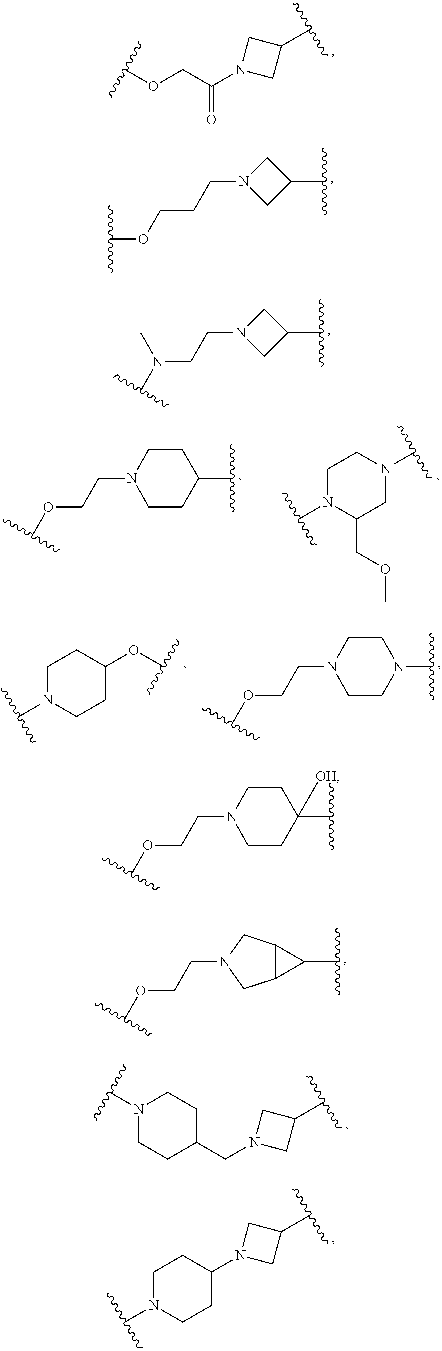

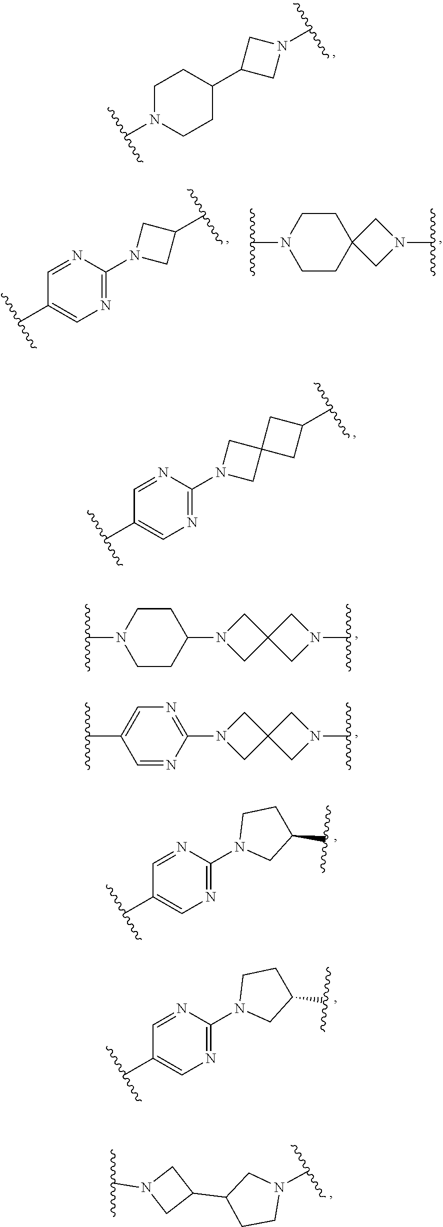

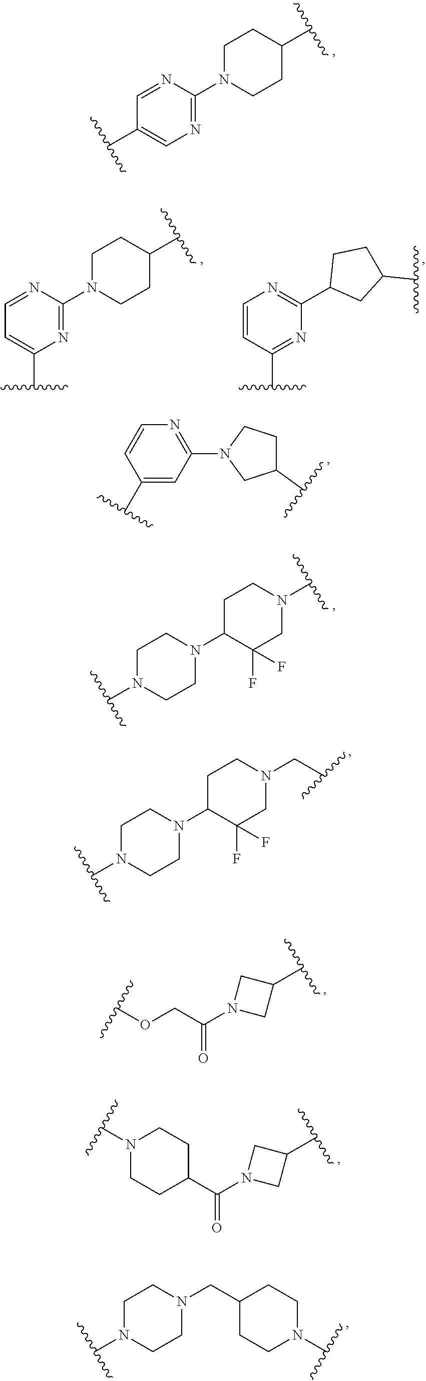

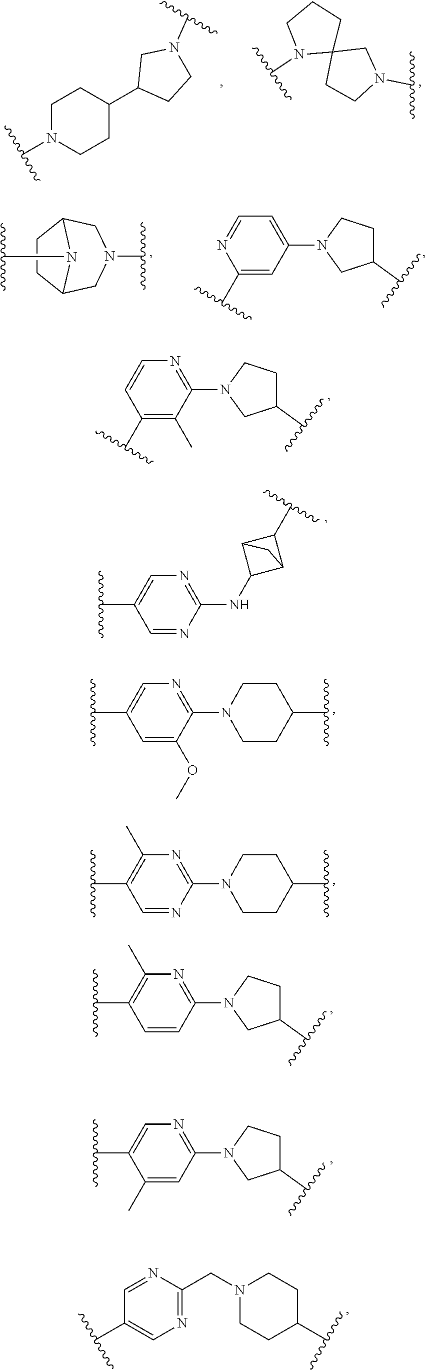

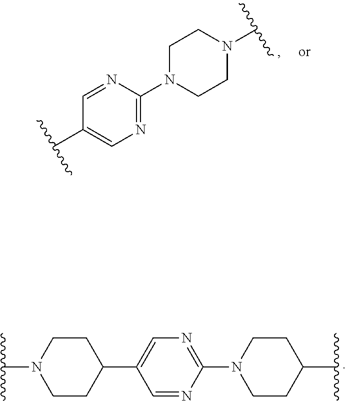 [SMILES: *c1cnc(N2CCN(*)CC2)nc1 ; *C1CCN(c2ncc(C3CCN(*)CC3)cn2)CC1]

In some embodiments, the linker has the structure of Formula III:

A¹-(B¹)_{f}-(C¹)_{g}-(B²)ₕ-(B³)ᵢ-(C²)ⱼ-(B⁴)ₖ-A², Formula III

wherein
A¹ is a bond between the linker and ring system A;
A² is a bond between the degradation moiety and the linker;
each of B¹, B², B³, and B⁴ is, independently, optionally substituted ethynyl, optionally substituted C₆-C₁₀ aryl, optionally substituted C₃-C₁₀ cycloalkyl, optionally substituted C₃-C₁₀ carbocyclyl, optionally substituted C₂-C₁₀ heterocyclyl, optionally substituted C₂-C₉ heteroaryl, O, S, S(O)₂, or NRN;
each R^{N} is, independently, H, optionally substituted C₁₋₄ alkyl, optionally substituted C₂₋₄ alkenyl, optionally substituted C₂₋₄ alkynyl, optionally substituted C₂₋₁₀ heterocyclyl, optionally substituted C₆₋₁₂ aryl, or optionally substituted C₁₋₇ heteroalkyl;
each of C¹ and C² is, independently, carbonyl, thiocarbonyl, sulphonyl, or phosphoryl; and
each of f, g, h, i, j, and k is, independently, 0 or 1.

In some embodiments, the linker is of structure -(L¹)ₙ-, wherein n is 1, 2, or 3, and each L¹ is independently O, NRN, ethynyl, optionally substituted C₂-C₁₀ heterocyclyl, optionally substituted C₂-C₉ heteroaryl, optionally substituted C₆-C₁₀ aryl, or optionally substituted C₃-C₁₀ cycloalkyl.

In some embodiments, at least one L¹ is optionally substituted C₂-C₁₀ heterocyclyl. In some embodiments the optionally substituted C₂-C₁₀ heterocyclyl is 4-, 5-, or 6-membered monocyclic heterocyclyl. In some embodiments the 4-, 5-, or 6-membered monocyclic heterocyclyl is:

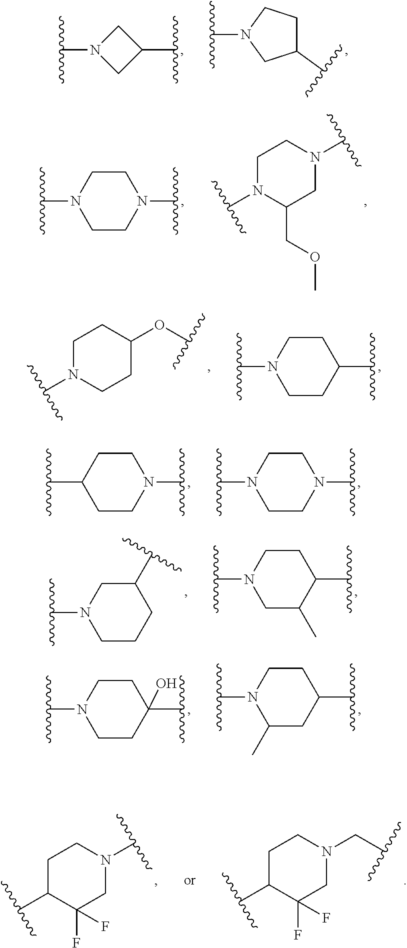

In some embodiments, the optionally substituted C₂-C₁₀ heterocyclyl is a spirocyclic heterocyclyl. In some embodiments, the spirocyclic heterocyclyl is:

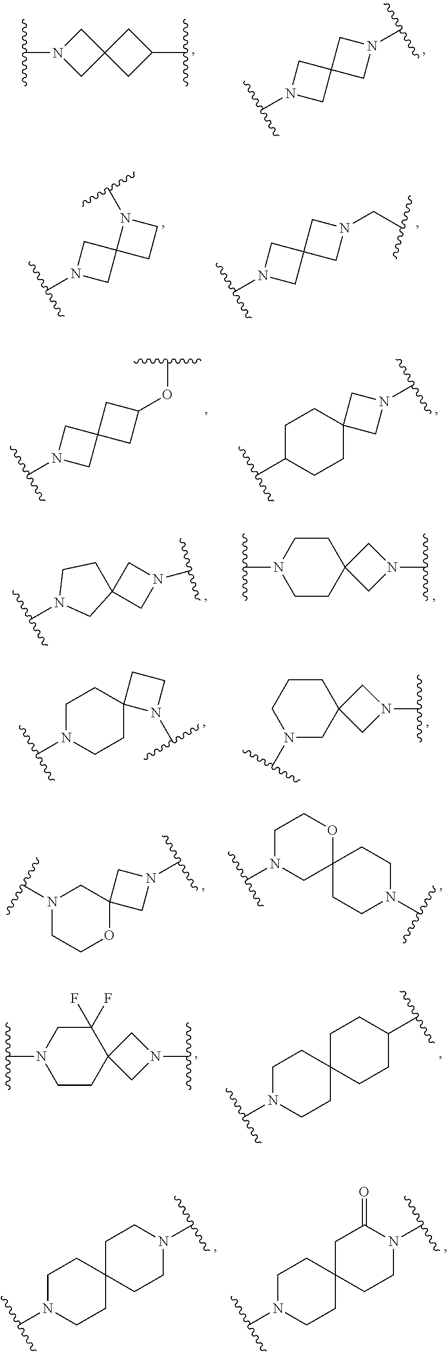

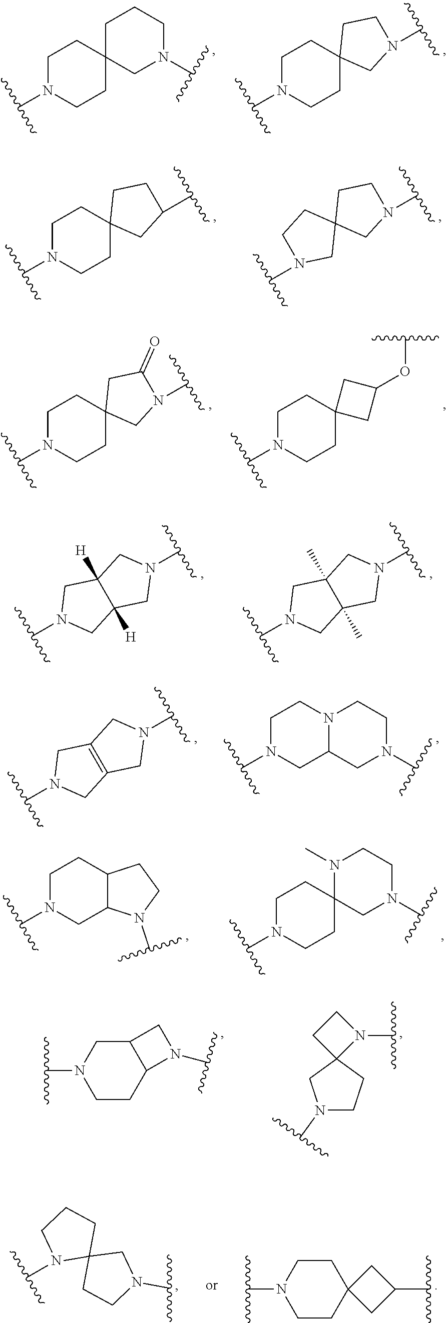

In some embodiments, the optionally substituted C₂-C₁₀ heterocyclyl is a bridged heterocyclyl. In some embodiments the bridged heterocyclyl is:

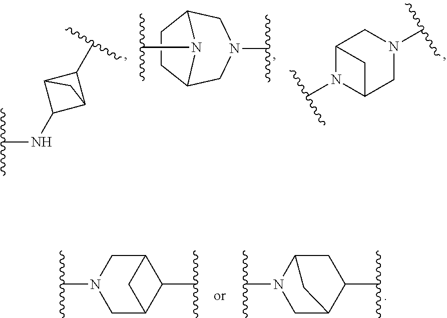 [SMILES: *NC1C2CC1C2* ; *N1CC2CCC(C1)N2* ; *N1CC2CC(C1)N2* ; *C1C2CC1CN(*)C2 ; *C1CC2CC1CN2*]

In some embodiments, the optionally C₂-C₁₀ heterocyclyl is a fused bicyclic heterocyclyl. In some embodiments, the fused bicyclic heterocyclyl is:

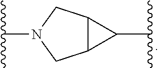 [SMILES: *C1C2CN(*)CC12]

In some embodiments, at least one L¹ is optionally substituted C₂-C₉ heteroaryl. In some embodiments, the linker is -(L¹)_{q}-(optionally substituted C₂-C₉ heteroaryl)-(L¹)_{q}-, wherein each q is independently 0 or 1. In some embodiments, the optionally substituted C₂-C₉ heteroaryl is a 6-membered monocyclic heteroaryl. In some embodiments, the 6-membered monocyclic heteroaryl is:

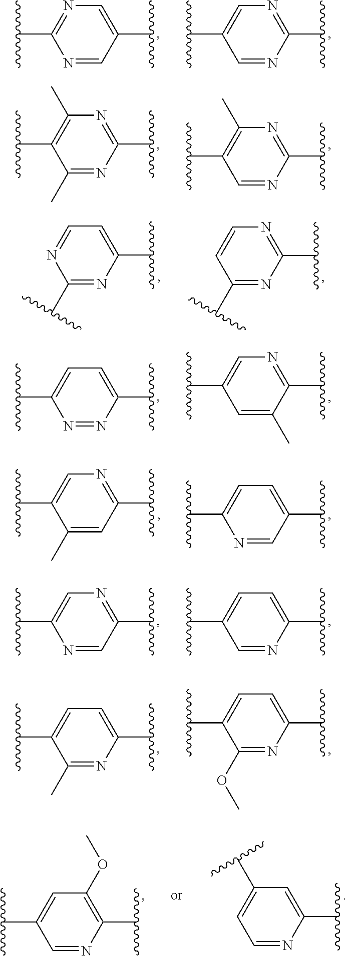

In some embodiments, at least one L¹ is optionally substituted C₂-C₉ heteroaryl. In some embodiments, the linker is:

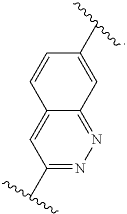 [SMILES: *c1ccc2cc(*)nnc2c1]

In some embodiments, at least one L¹ is optionally substituted C₆-C₁₀ aryl. In some embodiments, the optionally substituted C₆-C₁₀ aryl is a 6-membered monocyclic aryl. In some embodiments, the 6-membered monocyclic aryl is optionally substituted phenyl.

In some embodiments, at least one L¹ is optionally substituted C₃-C₁₀ cycloalkyl. In some embodiments, the optionally substituted C₃-C₁₀ cycloalkyl is a monocyclic cycloalkyl. In some embodiments, the 6-membered monocyclic cycloalkyl is:

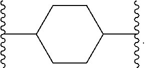 [SMILES: *C1CCC(*)CC1]

In some embodiments, the optionally substituted C₃-C₁₀ cycloalkyl is a bridged cycloalkyl. In some embodiments, the bridged cycloalkyl is:

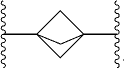 [SMILES: *C12CC(*)(C1)C2]

In some embodiments, at least one L¹ is ethynyl.

In some embodiments, one and only one L¹ is O. In some embodiments, one and only one L¹ is NR^{N}. In some embodiments, RN is optionally substituted C₁-C₄ alkyl. In some embodiments, R^{N} is H.

In some embodiments, the linker is of the following structure:

A¹-(B¹)_{f}-(B²)ₕ-(B³)ᵢ-(B⁴)ₖ-A²,

wherein each of B¹, B², B³, and B⁴ is, independently, optionally substituted ethynyl, optionally substituted C₆-C₁₀ aryl, optionally substituted C₃-C₁₀ cycloalkyl, optionally substituted C₂-C₁₀ heterocyclyl, optionally substituted C₂-C₉ heteroaryl, O, or NRN.

In some embodiments, at least one of f, h, i, and k is 1.

In some embodiments, each of B¹, B², B³, and B⁴ is, independently, O, ethynyl, optionally substituted C₂-C₉ heteroaryl, optionally substituted C₂-C₁₀ heterocyclyl, optionally substituted C₃-C₁₀ cycloalkyl, or optionally substituted C₆-C₁₀ aryl. In some embodiments, each of B¹, B², B³, and B⁴ is, independently optionally substituted C₂-C₉ heteroaryl or optionally substituted C₂-C₁₀ heterocyclyl. In some embodiments, each of B¹ and B⁴ is, independently,

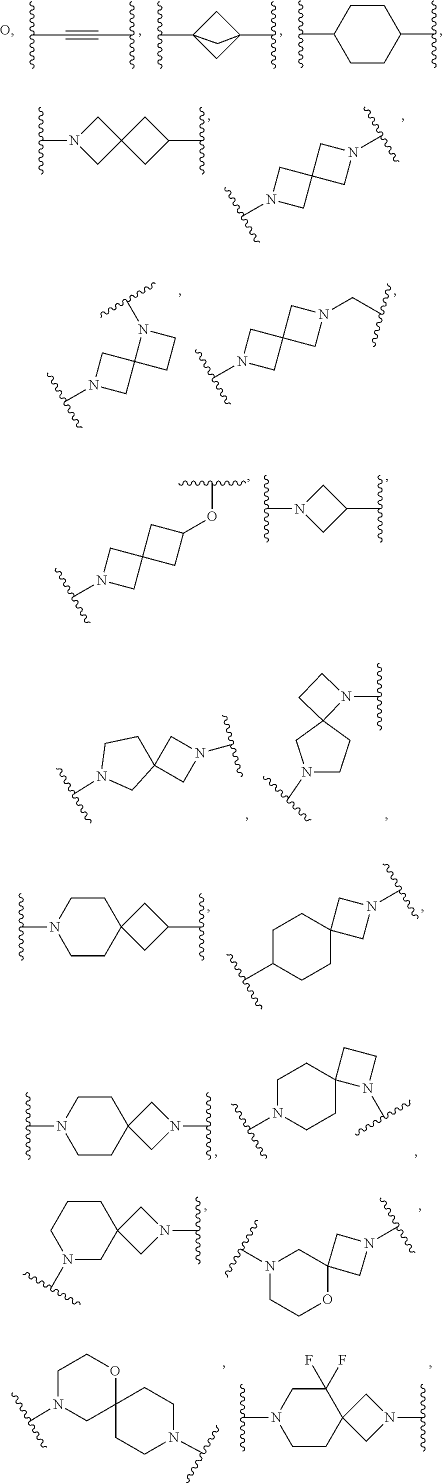

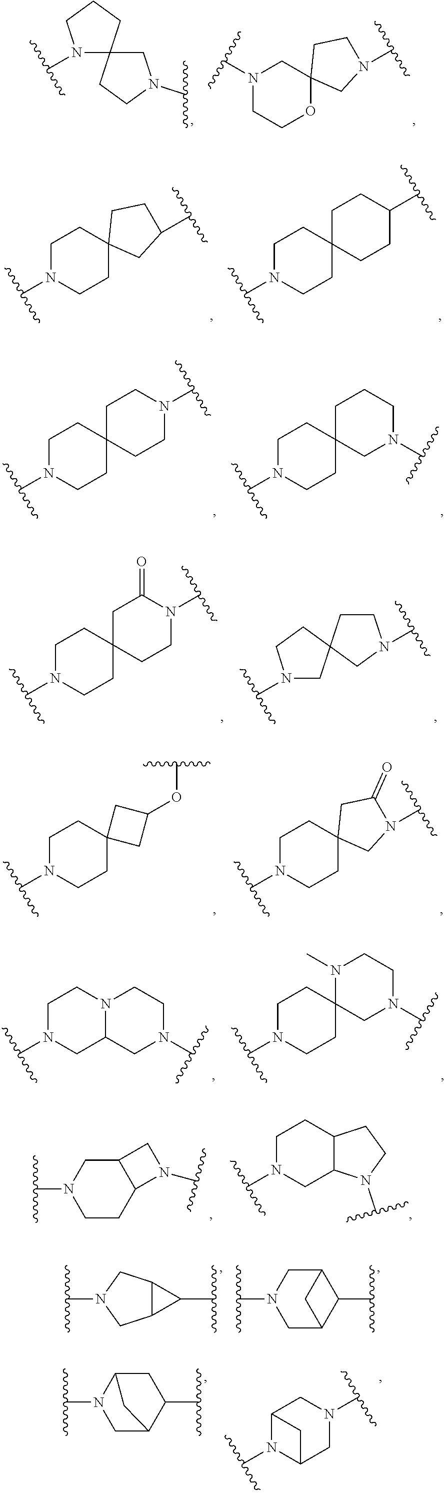

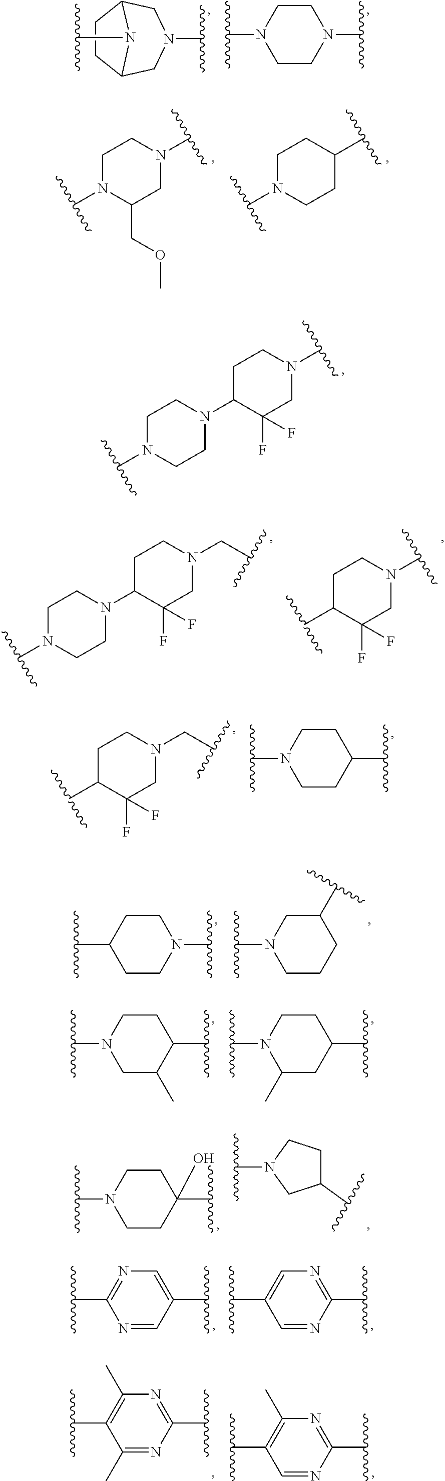

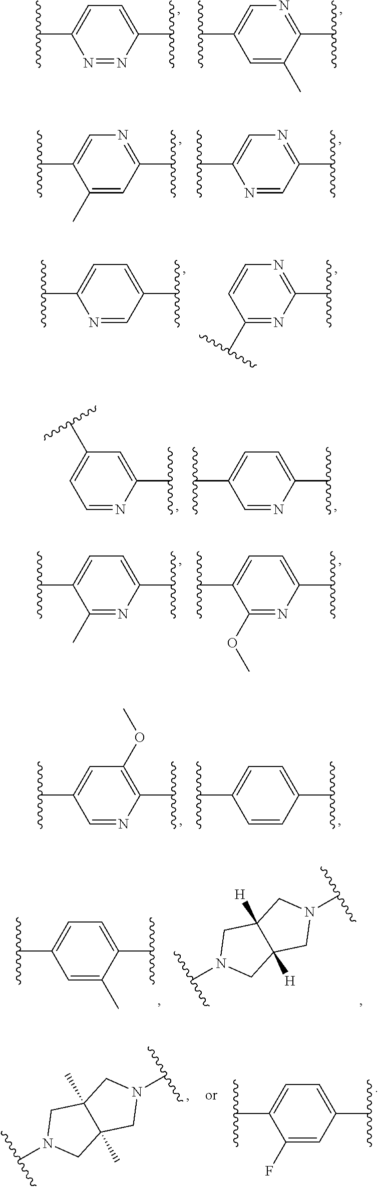

In some embodiments, B¹ is:

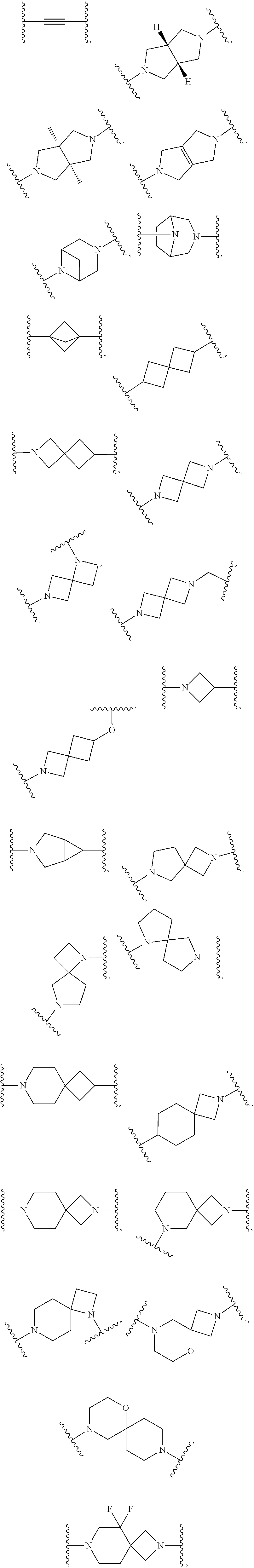

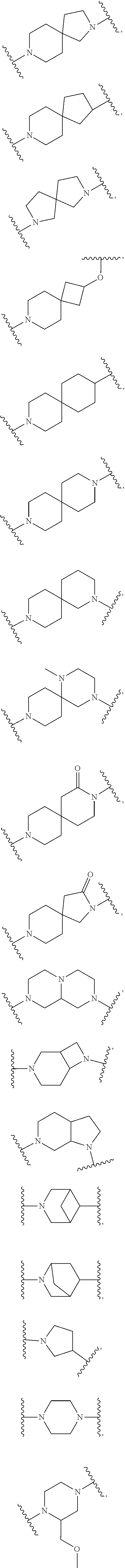

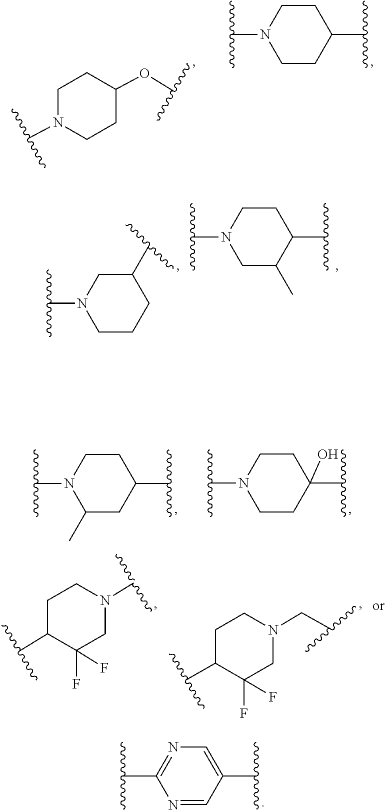

In some embodiments, B⁴ is:

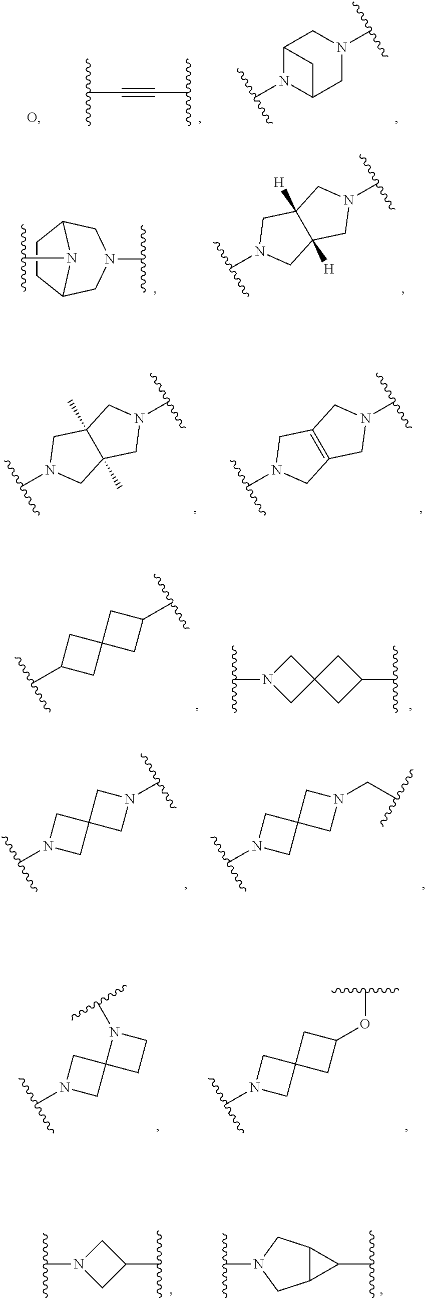

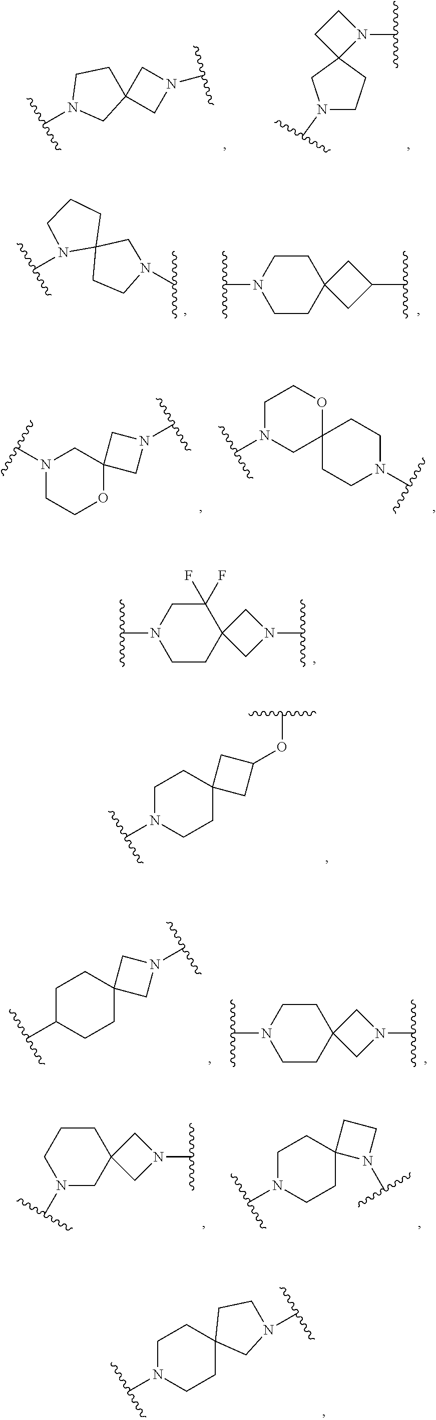

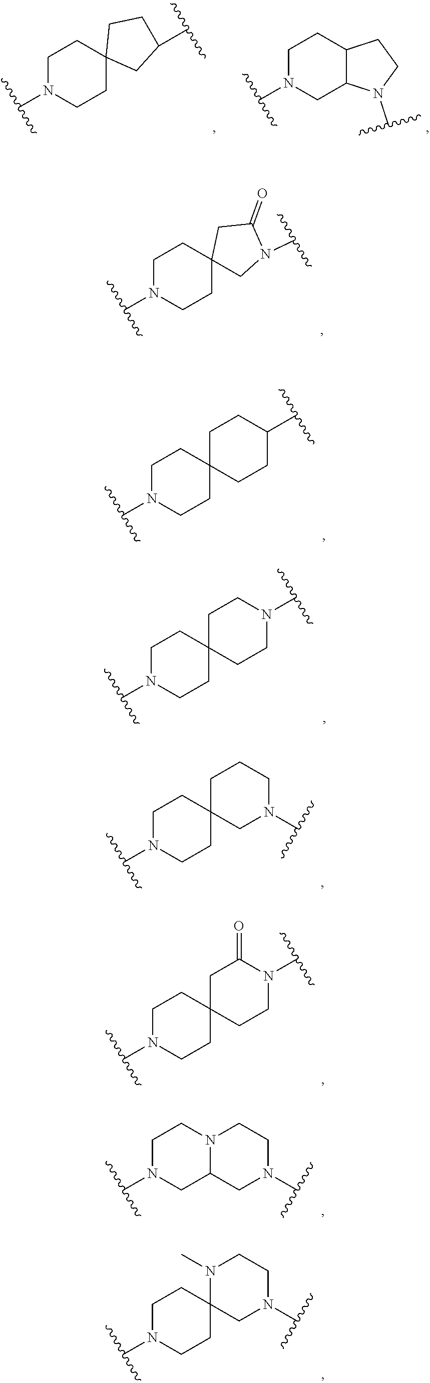

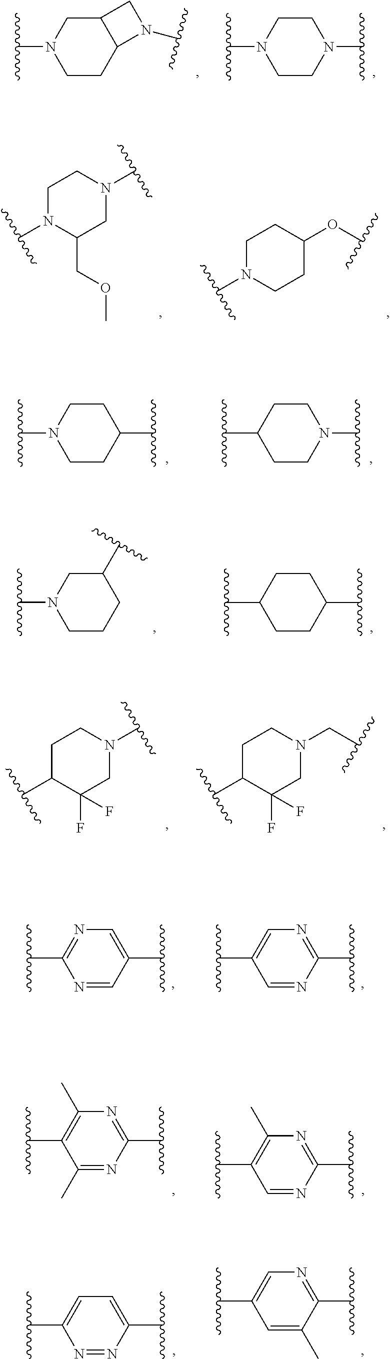

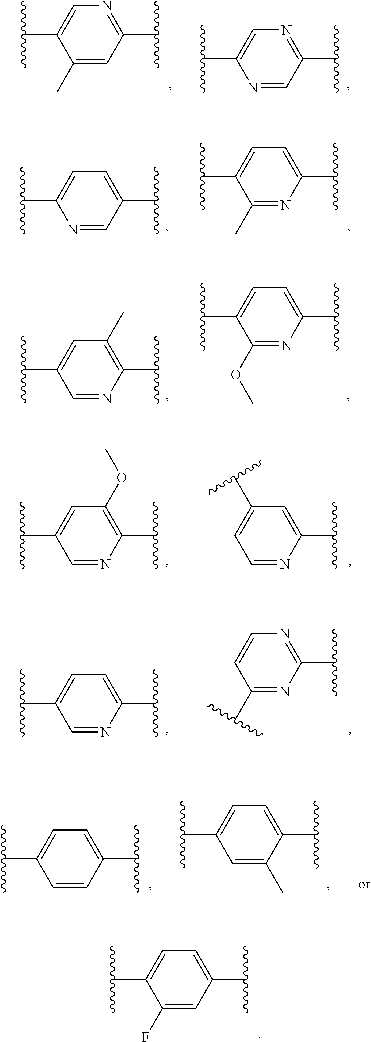

In some embodiments, B² is NR^{N}. In some embodiments, B² is NH. In some embodiments, B² is optionally substituted C₂-C₉ heteroaryl. In some embodiments, B² is:

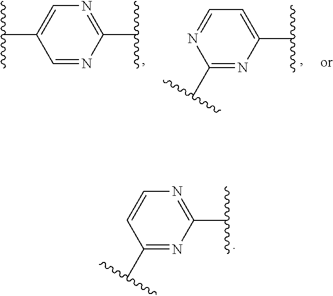 [SMILES: *c1cnc(*)nc1 ; *c1ccnc(*)n1 ; *c1ccnc(*)n1]

In some embodiments, f is 0. In some embodiments, f is 1. In some embodiments, g is 0. In some embodiments, g is 1. In some embodiments, h is 0. In some embodiments, h is 1. In some embodiments, i is 0. In some embodiments, i is 1. In some embodiments, j is 0. In some embodiments, j is 1. In some embodiments, k is 0. In some embodiments, k is 1.

In some embodiments, the linker has the structure of

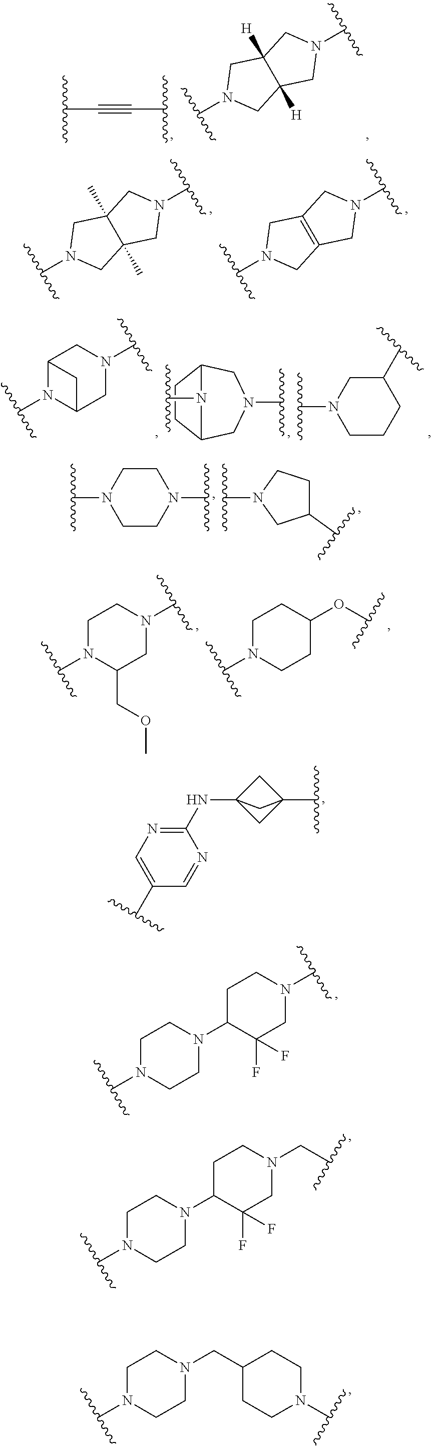

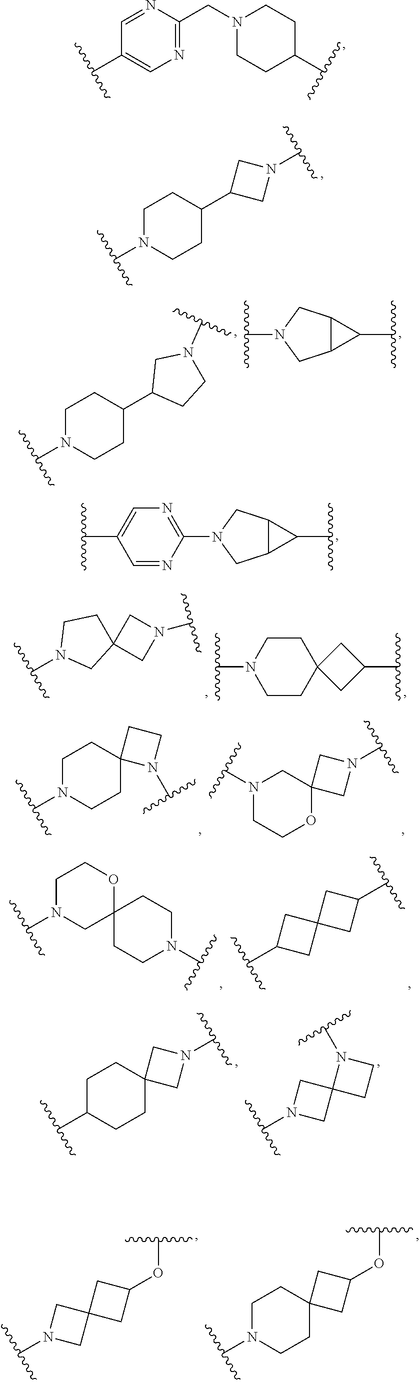

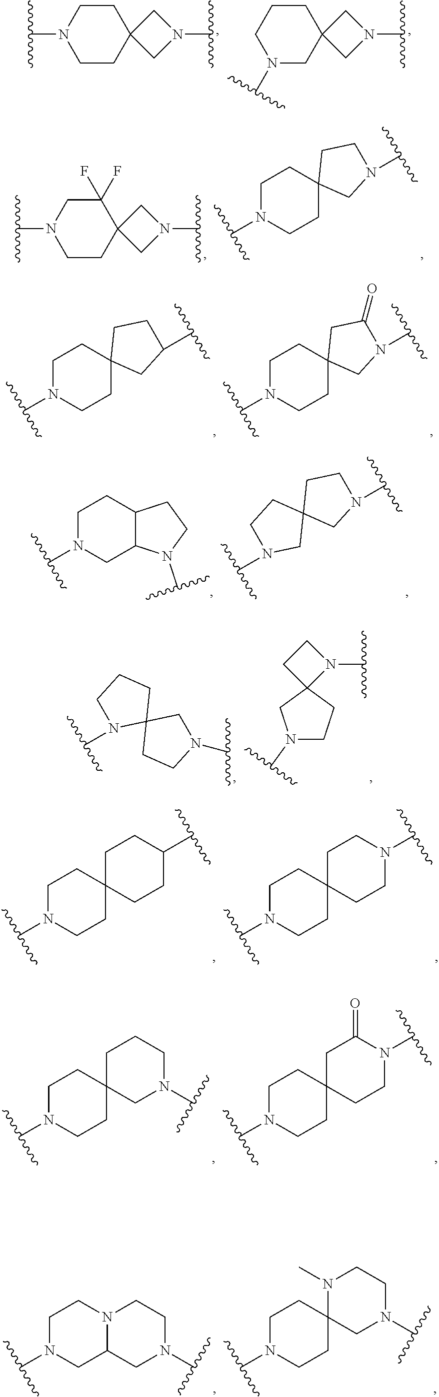

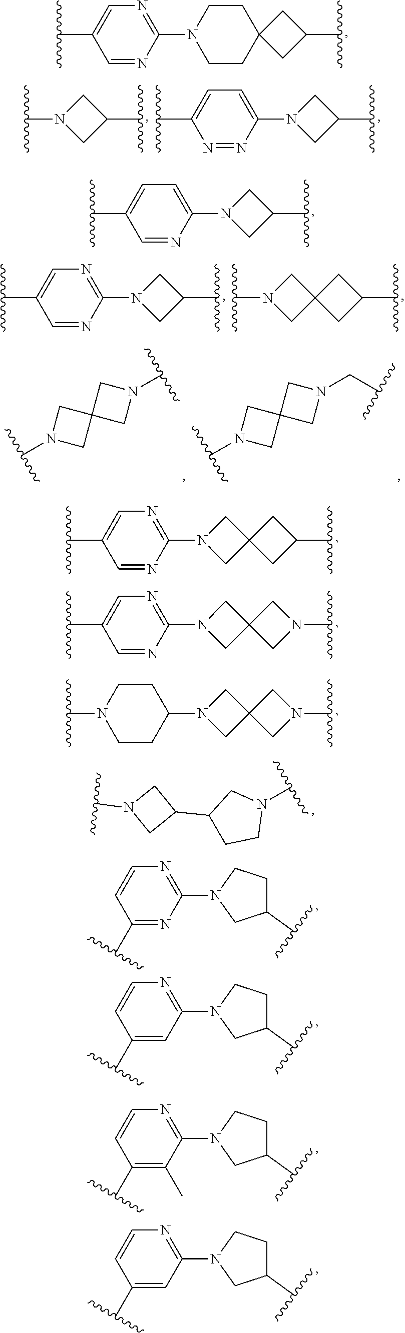

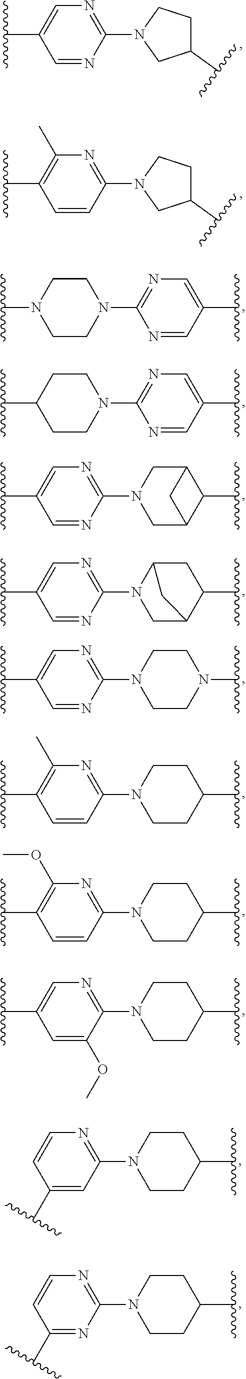

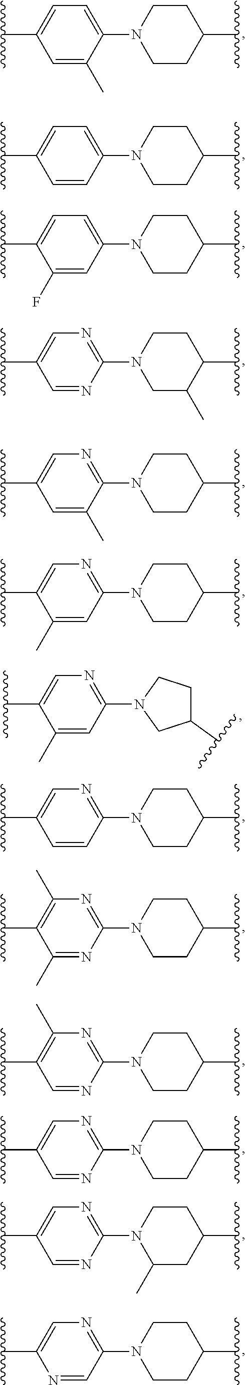

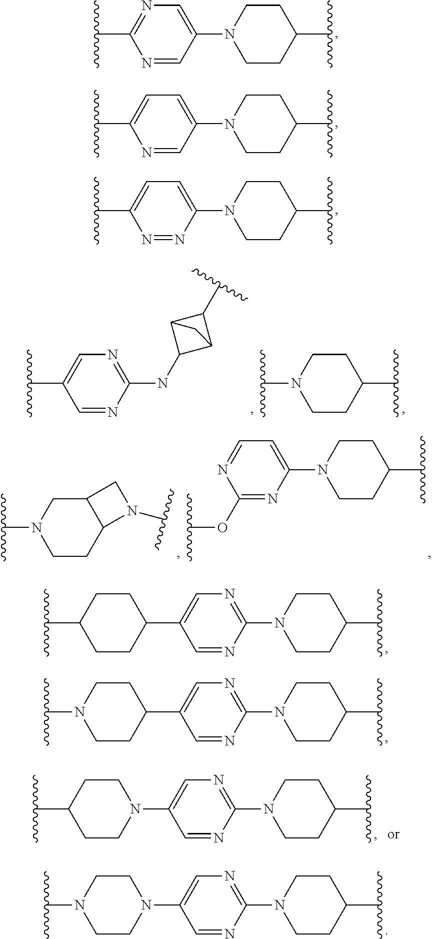

In some embodiments, the shortest chain of atoms connecting two valencies of the linker is 2 to 10 atoms long. In some embodiments, the shortest chain of atoms connecting two valencies of the linker is 6 atoms long.

In some embodiments, the linker has a structure of the linker in any one of compounds 1-121 in Table 1 (e.g., of any of the compounds with a ratio of BRG1 IC₅₀ to BRM IC₅₀ of at least 5 (e.g., at least 7, 10, 15, 20, 25, or 30)). In some embodiments, the linker has a structure of the linker in any one of compounds 1-121 in Table 1 (e.g., of any of the compounds with a BRM IC₅₀ of ++ or better (e.g., +++ or ++++(e.g., ++++))). In some embodiments, the linker has a structure of the linker in any one of compounds 1-121 in Table 1 (e.g., of any of the compounds with a BRM IC₅₀ of ++ or better (e.g., +++ or ++++(e.g., ++++)) and with a ratio of BRG1 IC₅₀ to BRM IC₅₀ of at least 5 (e.g., at least 7, 10, 15, 20, 25, or 30)).

In an aspect, the invention features a compound selected from the group consisting of 1-121 in Table 1 and pharmaceutically acceptable salts thereof. In some embodiments, the compound is any one of compounds 1-121 in Table 1 with a ratio of BRG1 IC₅₀ to BRM IC₅₀ of at least 5 (e.g., at least 7, 10, 15, 20, 25, or 30) or a pharmaceutically acceptable salt thereof. In some embodiments, the compound is any one of compounds 1-121 in Table 1 with a BRM IC₅₀ of ++ or better as found in Table 15 (e.g., +++ or ++++(e.g., ++++)) or a pharmaceutically acceptable salt thereof. In some embodiments, the compound is any one of compounds 1-121 in Table 1 a BRM IC₅₀ of ++ or better as found in Table 15 (e.g., +++ or ++++(e.g., ++++)) and with a ratio of BRG1 IC₅₀ to BRM IC₅₀ of at least 5 (e.g., at least 7, 10, 15, 20, 25, or 30) or a pharmaceutically acceptable salt thereof.

TABLE 1
Compounds of the invention

| # | Compound |
|---|---|
| 1 | 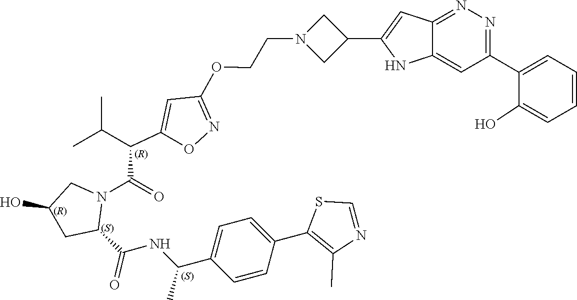 |
| 2 | 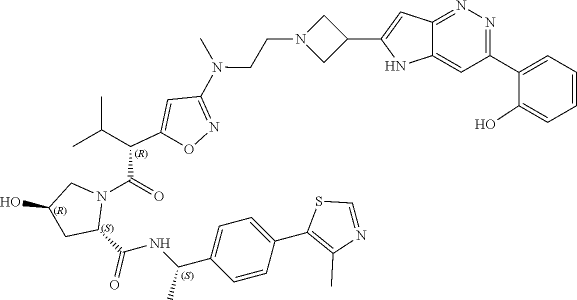 |
| 3 | 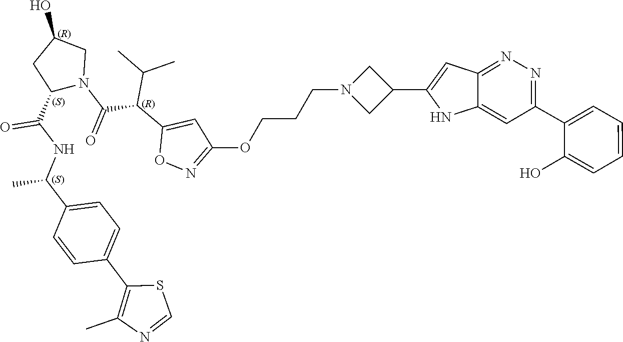 |
| 4 | 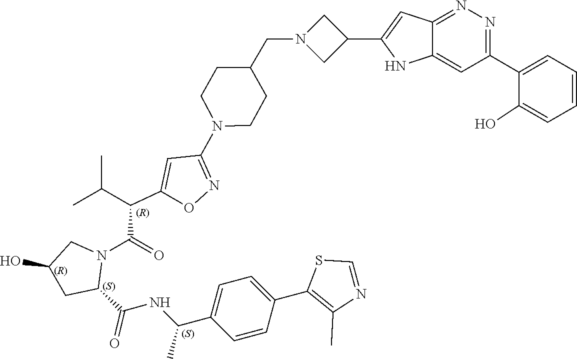 |
| 5 | 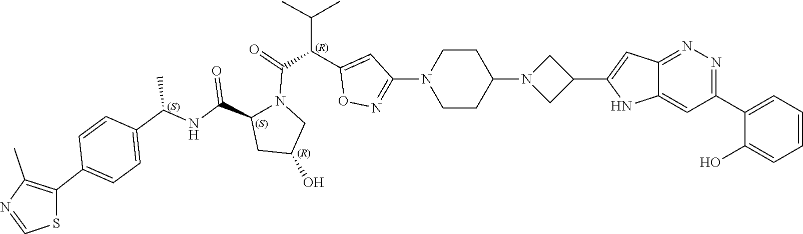 |
| 6 | 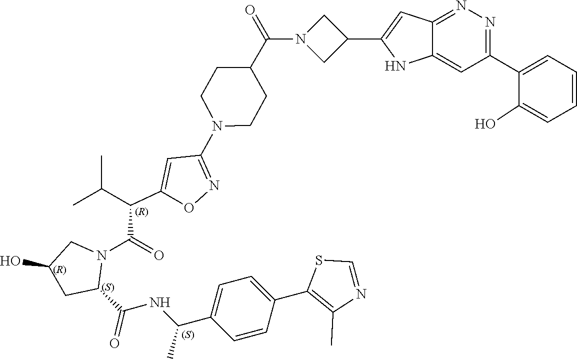 |
| 7 | 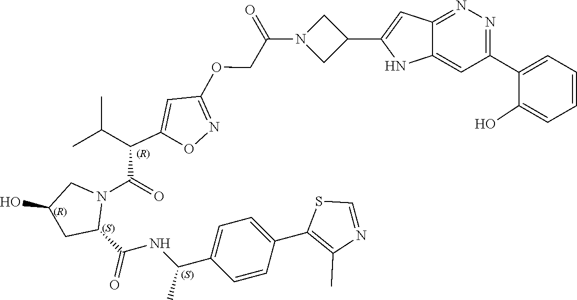 |
| 8 | 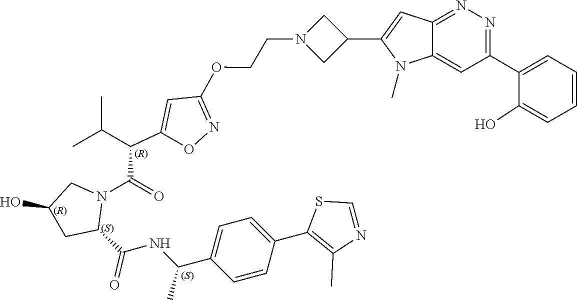 |
| 9 | 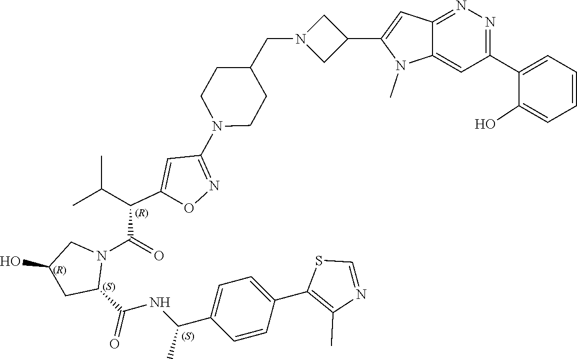 |
| 10 | 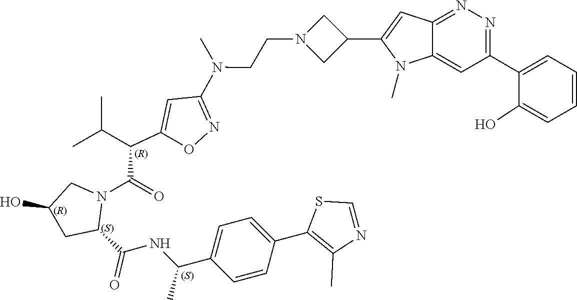 |
| 11 | 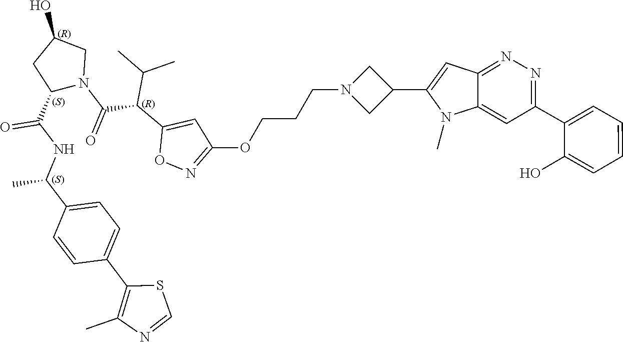 |
| 12 | 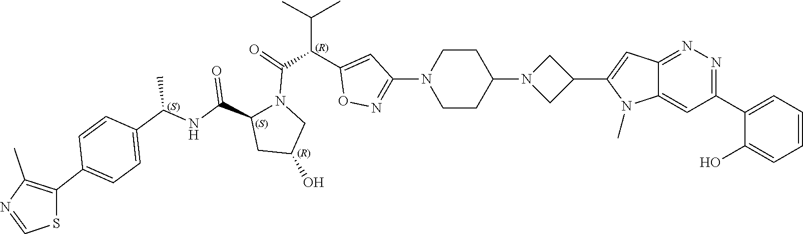 |
| 13 | 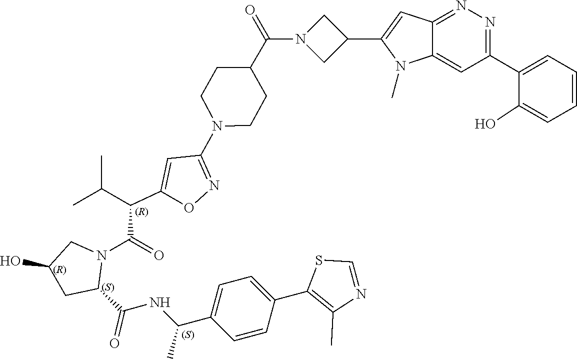 |
| 14 | 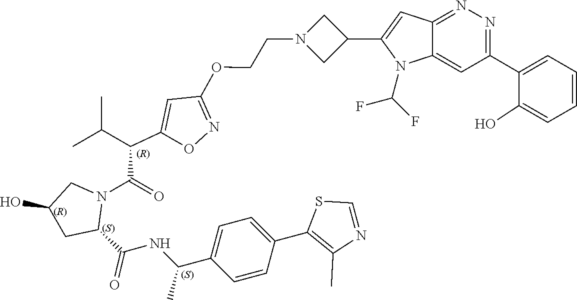 |
| 15 | 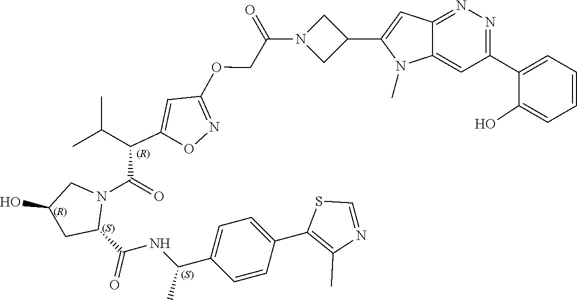 |
| 16 | 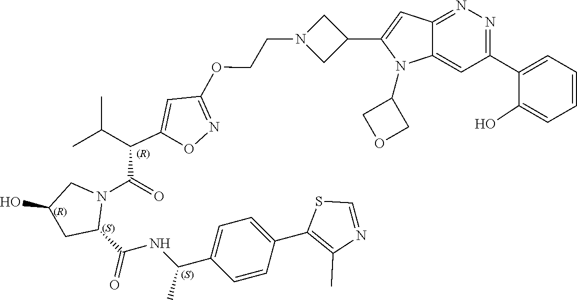 |
| 17 | 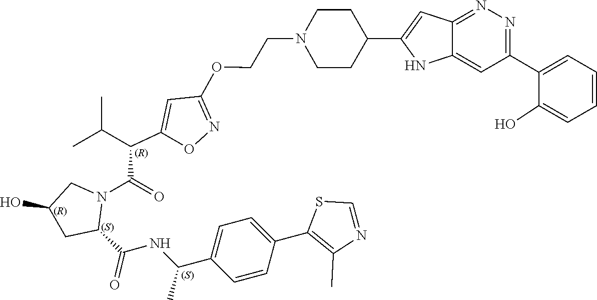 |
| 18 | 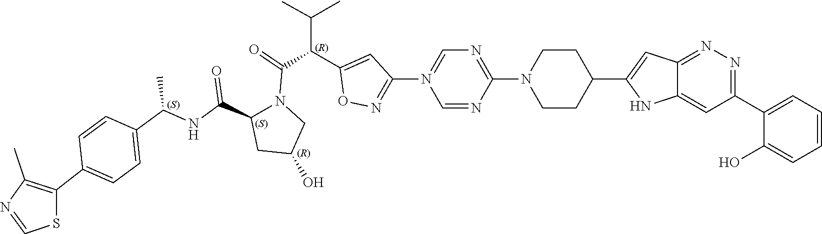 |
| 19 | 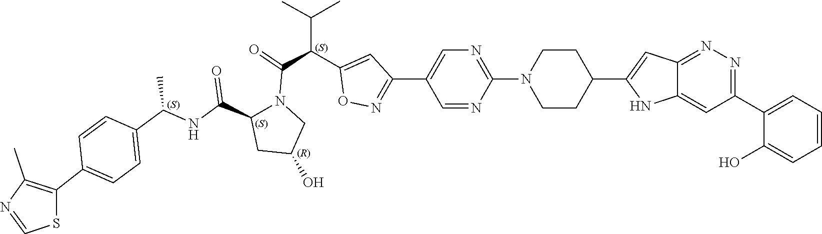 |
| 20 | 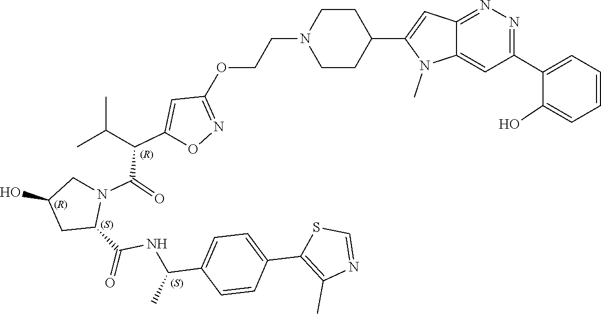 |
| 21 | 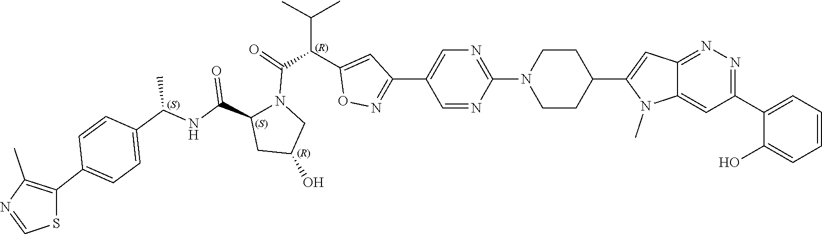 |
| 22 | 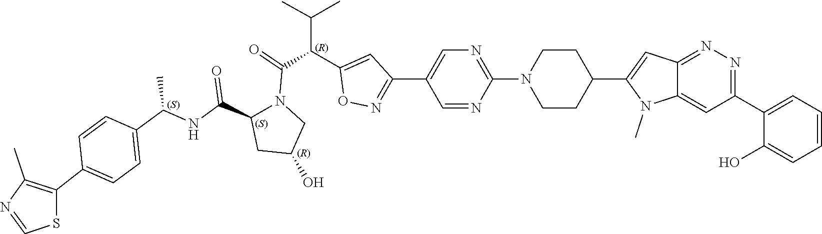 |
| 23 | 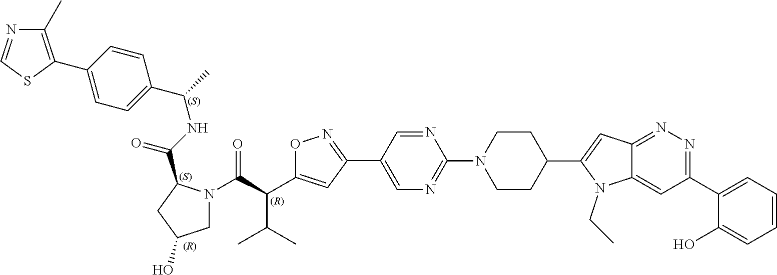 |
| 24 | 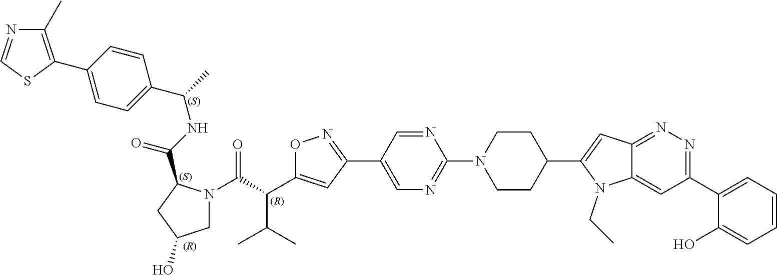 |
| 25 | 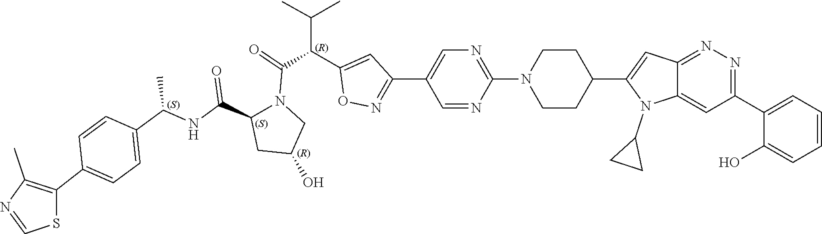 |
| 26 | 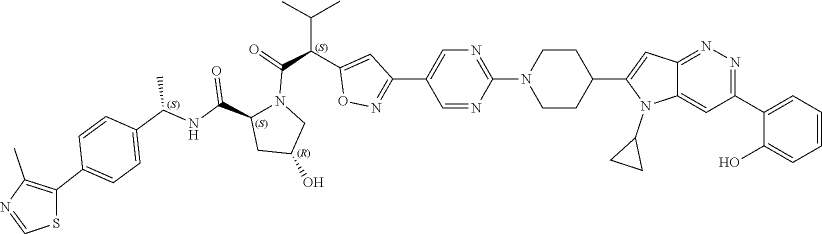 |
| 27 | 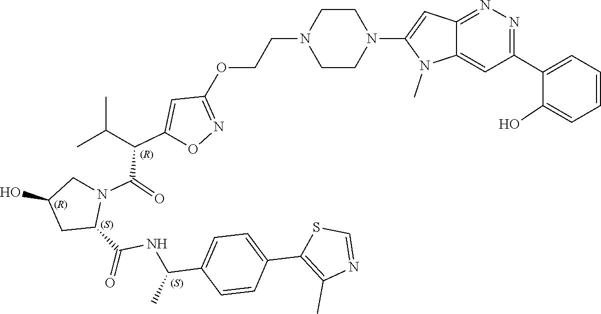 |
| 28 | 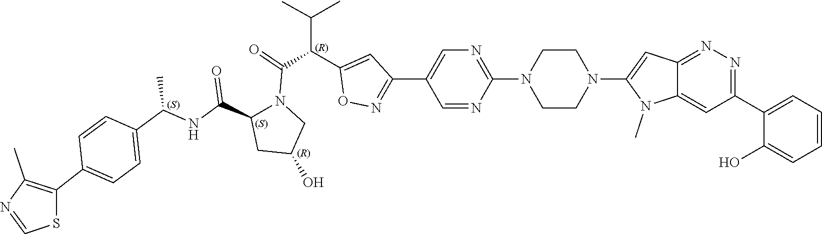 |
| 29 | 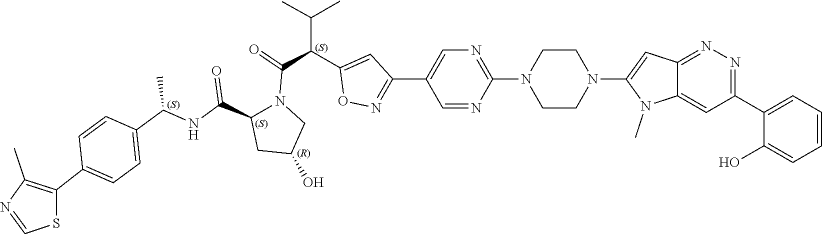 |
| 30 | 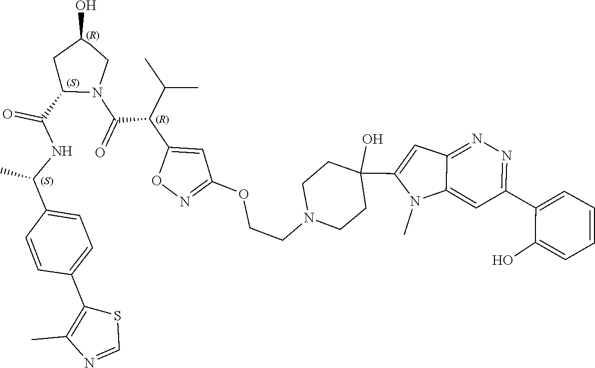 |
| 31 | 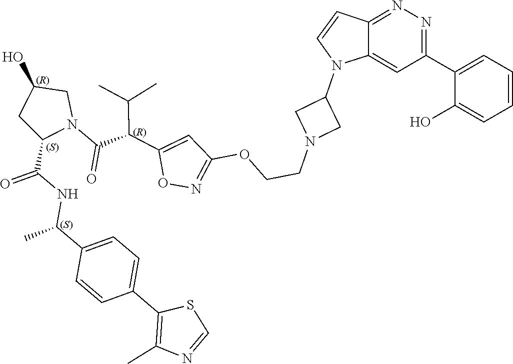 |
| 32 | 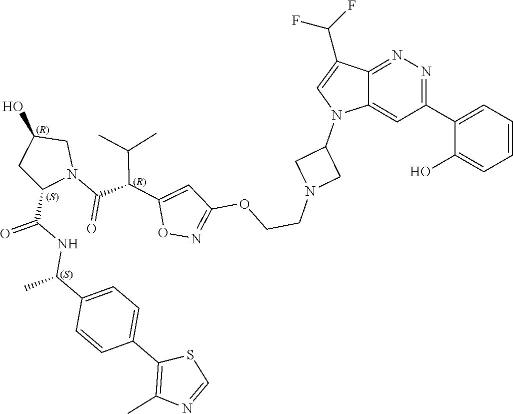 |
| 33 | 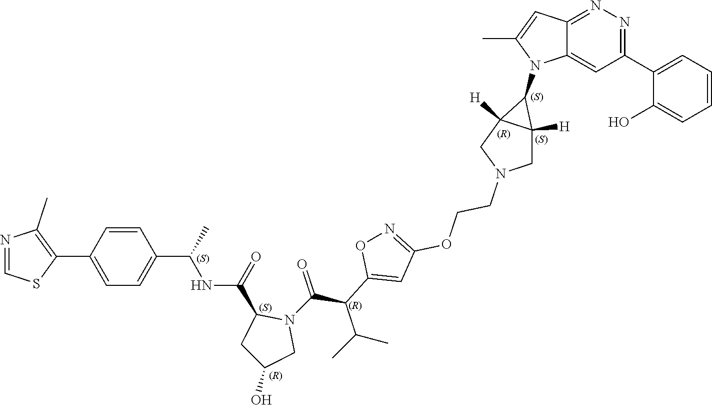 |
| 34 | 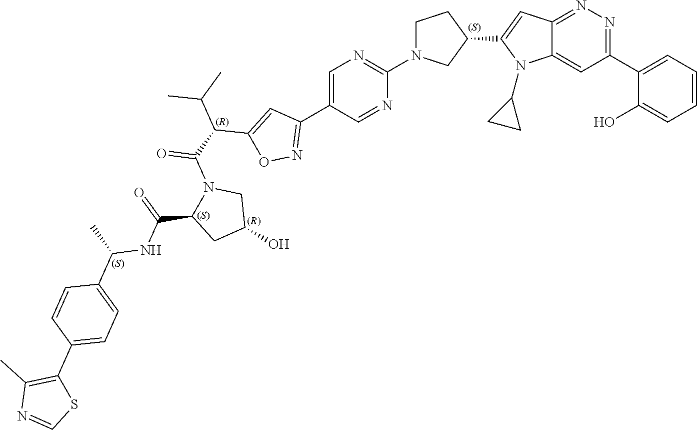 |
| 35 | 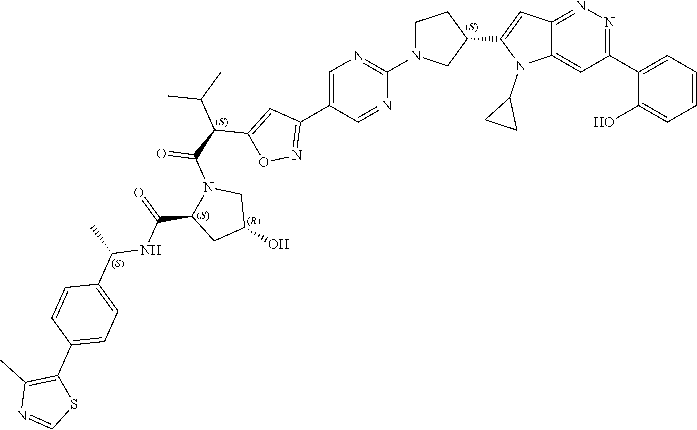 |
| 36 | 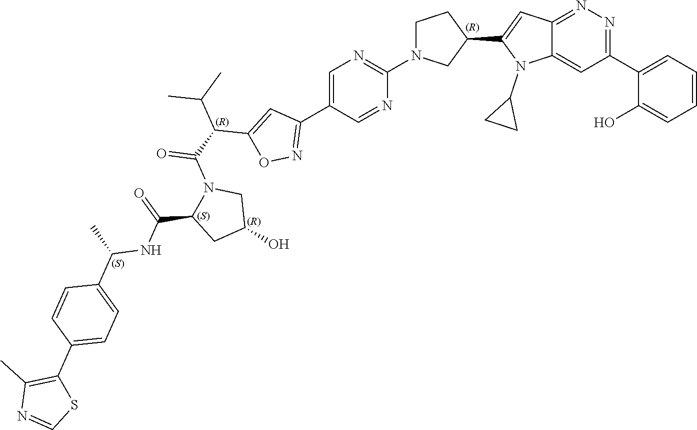 |
| 37 | 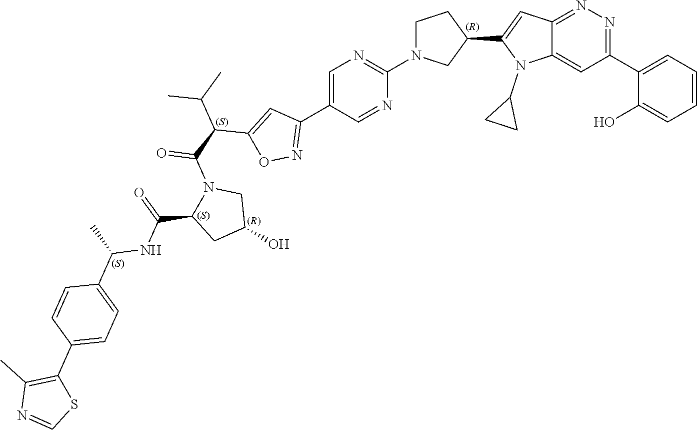 |
| 38 | 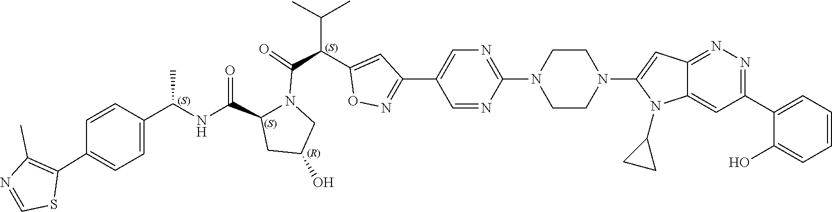 |
| 39 | 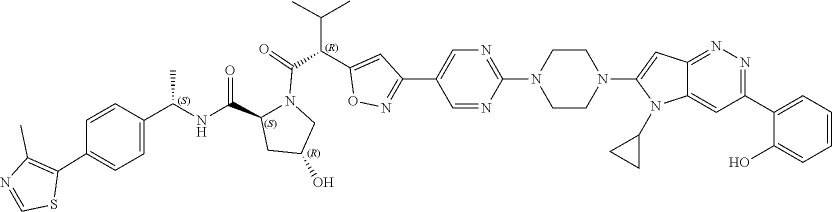 |
| 40 | 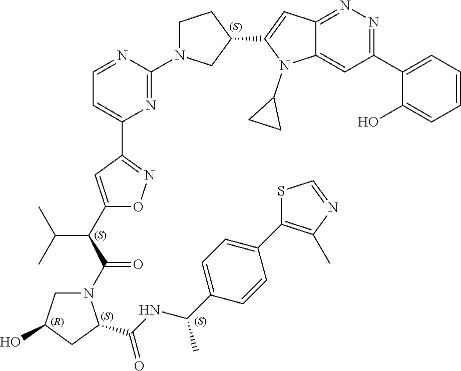 |
| 41 | 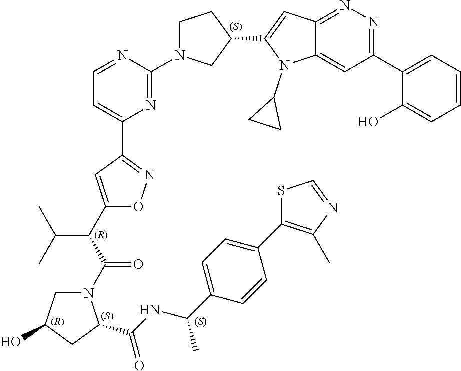 |
| 42 | 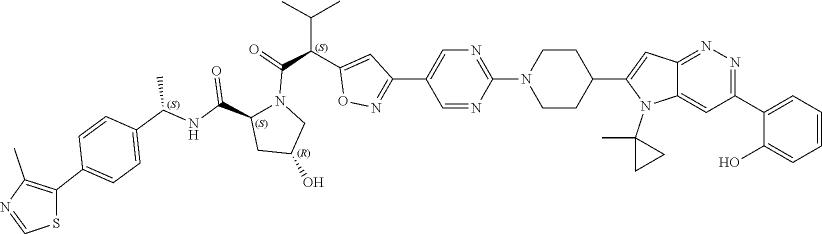 |
| 43 | 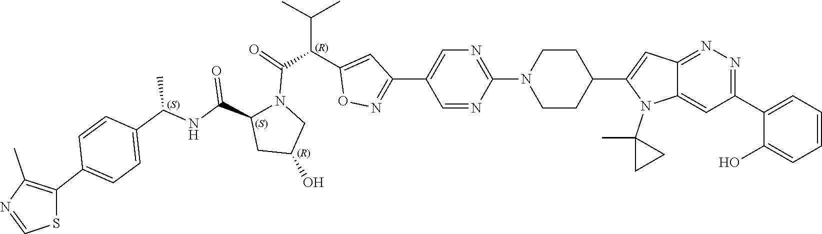 |
| 44 | 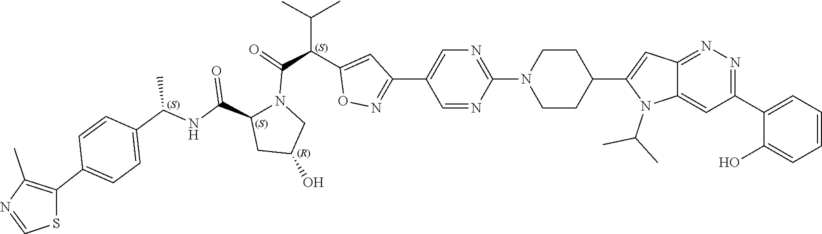 |
| 45 | 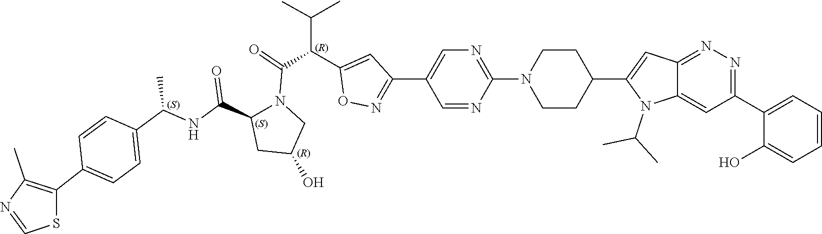 |
| 46 | 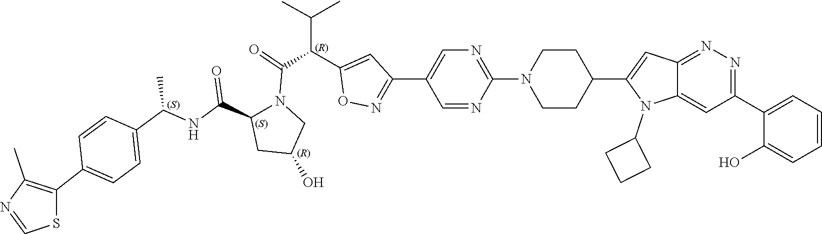 |
| 47 | 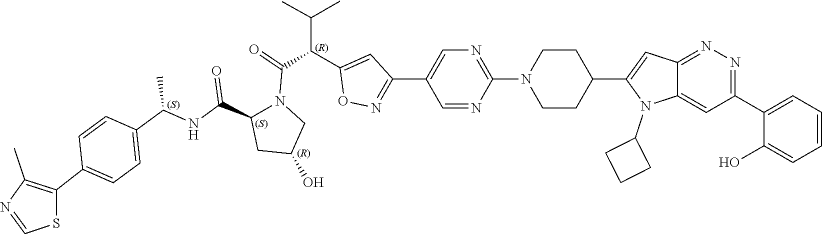 |
| 48 | 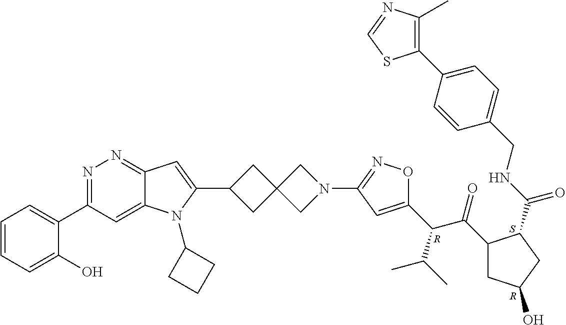 |
| 49 | 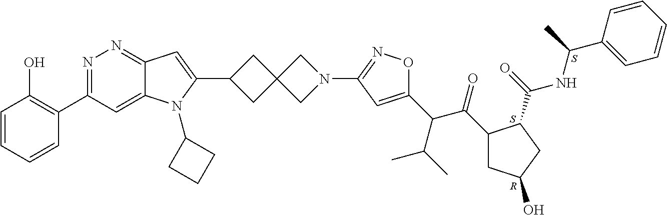 |
| 50 | 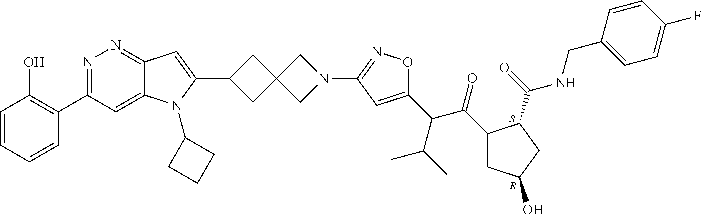 |
| 51 | 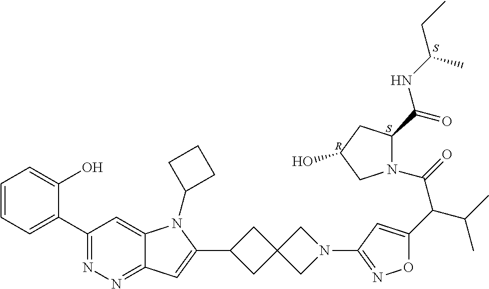 |
| 52 | 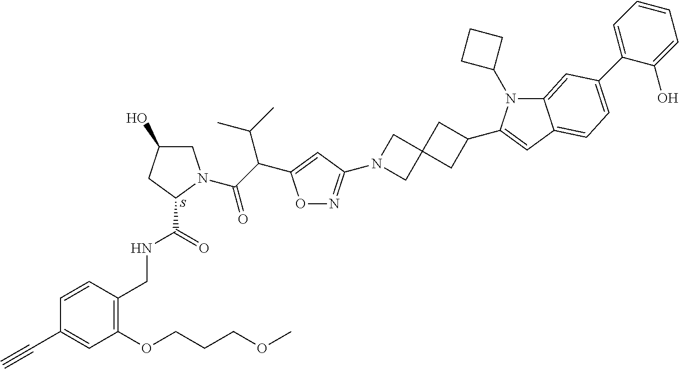 |
| 53 | 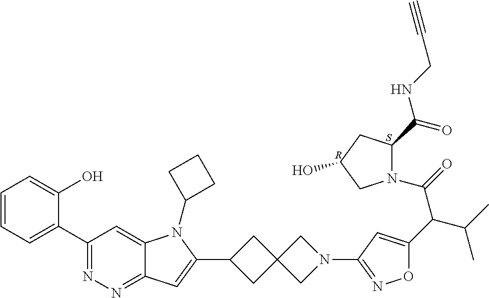 |
| 54 | 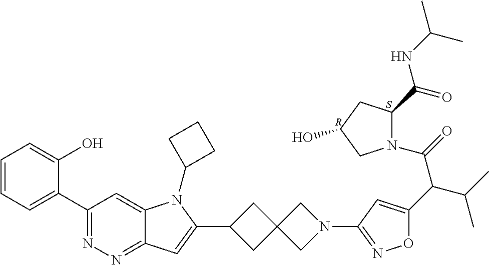 |
| 55 | 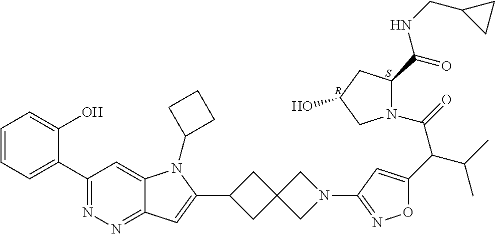 |
| 56 | 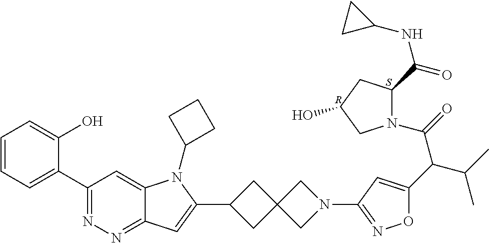 |
| 57 | 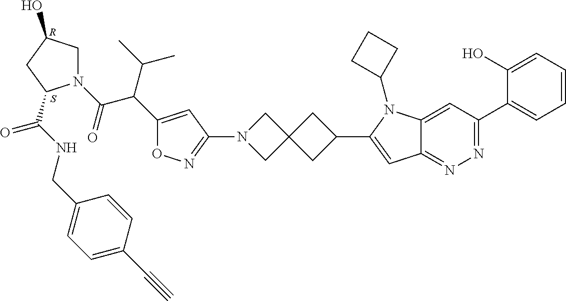 |
| 58 | 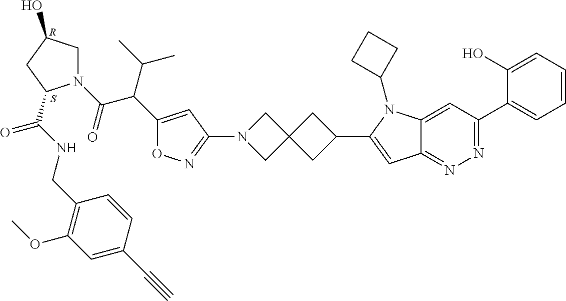 |
| 59 | 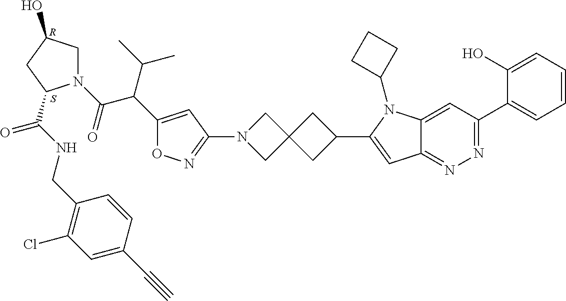 |
| 60 | 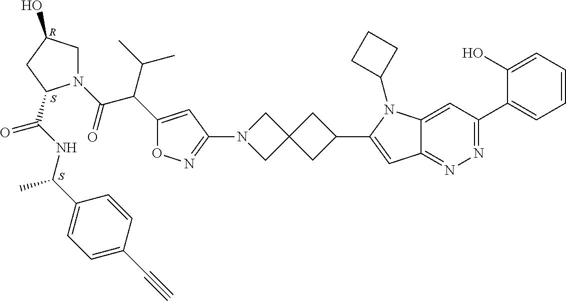 |
| 61 | 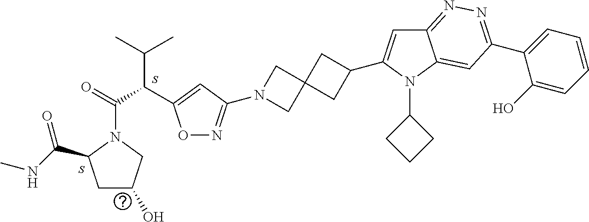 |
| 62 | 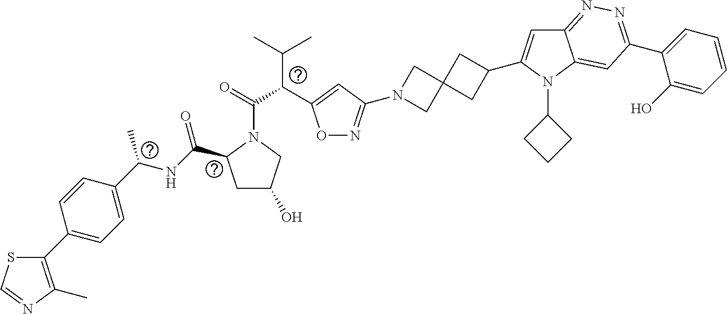 [SMILES: Cc1ncsc1-c1ccc([C@H](C)NC(=O)[C@@H]2C[C@@H](O)CN2C(=O)[C@@H](c2cc(N3CC4(CC(c5cc6nnc(-c7ccccc7O)cc6n5C5CCC5)C4)C3)no2)C(C)C)cc1] |
| 63 | 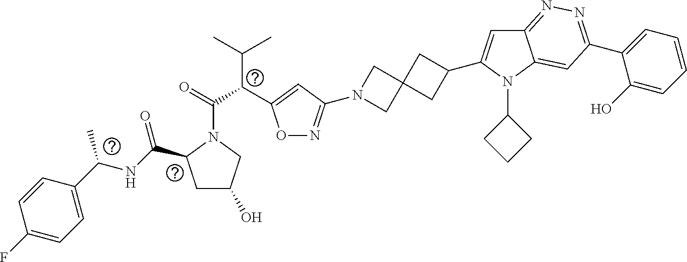 [SMILES: CC(C)[C@@H](C(=O)N1C[C@H](O)C[C@H]1C(=O)N[C@@H](C)c1ccc(F)cc1)c1cc(N2CC3(CC(c4cc5nnc(-c6ccccc6O)cc5n4C4CCC4)C3)C2)no1] |
| 64 | 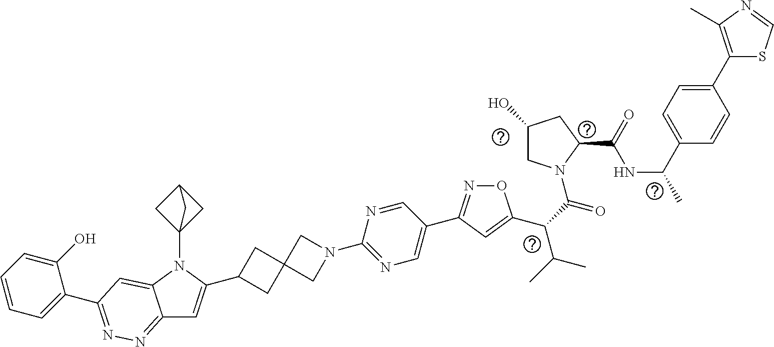 [SMILES: Cc1ncsc1-c1ccc([C@H](C)NC(=O)[C@@H]2C[C@@H](O)CN2C(=O)[C@@H](c2cc(-c3cnc(N4CC5(CC(c6cc7nnc(-c8ccccc8O)cc7n6C67CC(C6)C7)C5)C4)nc3)no2)C(C)C)cc1] |
| 65 | 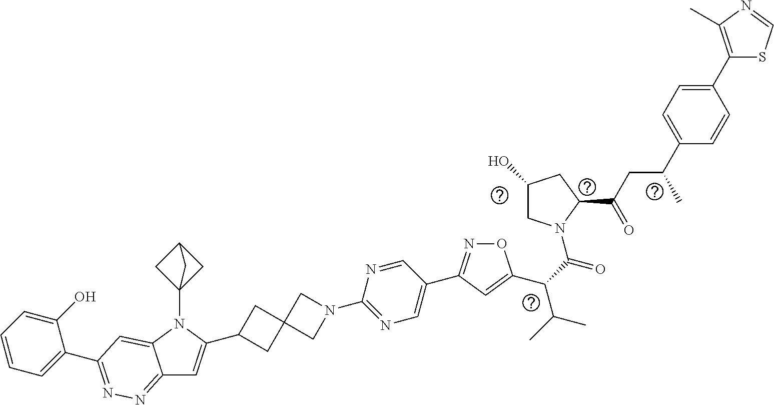 [SMILES: Cc1ncsc1-c1ccc([C@H](C)CC(=O)[C@@H]2C[C@@H](O)CN2C(=O)[C@@H](c2cc(-c3cnc(N4CC5(CC(c6cc7nnc(-c8ccccc8O)cc7n6C67CC(C6)C7)C5)C4)nc3)no2)C(C)C)cc1] |
| 66 | 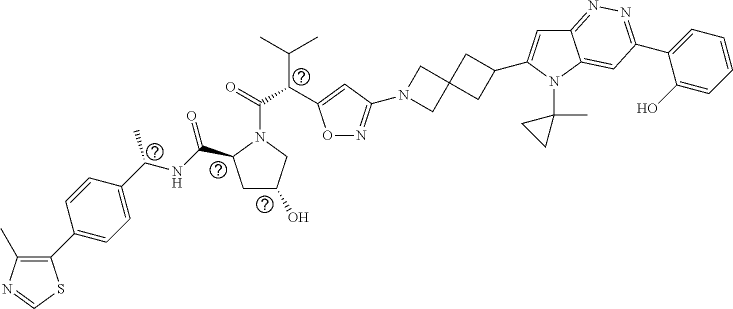 [SMILES: Cc1ncsc1-c1ccc([C@H](C)NC(=O)[C@@H]2C[C@@H](O)CN2C(=O)[C@@H](c2cc(N3CC4(CC(c5cc6nnc(-c7ccccc7O)cc6n5C5(C)CC5)C4)C3)no2)C(C)C)cc1] |
| 67 | 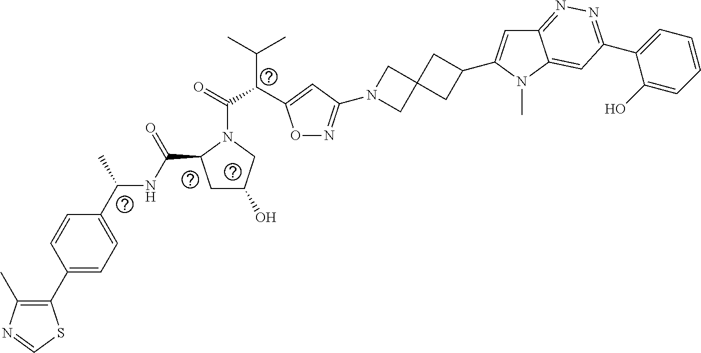 [SMILES: Cc1ncsc1-c1ccc([C@H](C)NC(=O)[C@@H]2C[C@@H](O)CN2C(=O)[C@@H](c2cc(N3CC4(CC(c5cc6nnc(-c7ccccc7O)cc6n5C)C4)C3)no2)C(C)C)cc1] |
| 68 | 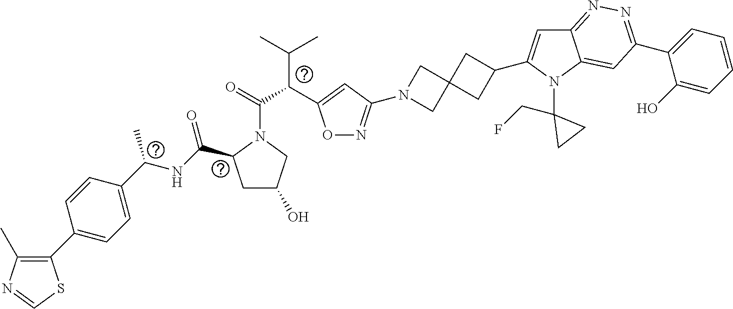 [SMILES: Cc1ncsc1-c1ccc([C@H](C)NC(=O)[C@@H]2C[C@@H](O)CN2C(=O)[C@@H](c2cc(N3CC4(CC(c5cc6nnc(-c7ccccc7O)cc6n5C5(CF)CC5)C4)C3)no2)C(C)C)cc1] |
| 69 | 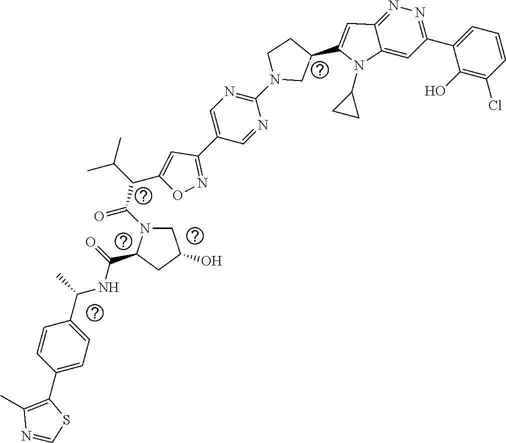 |
| 70 | 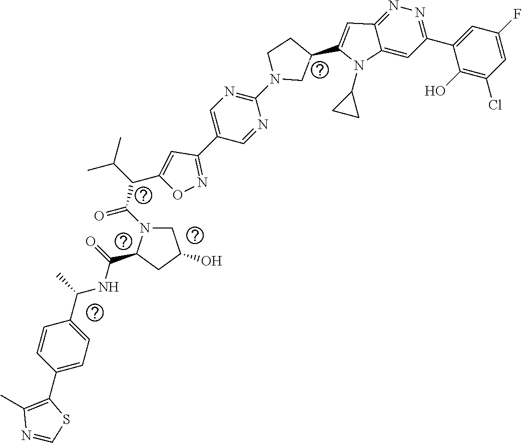 |
| 71 | 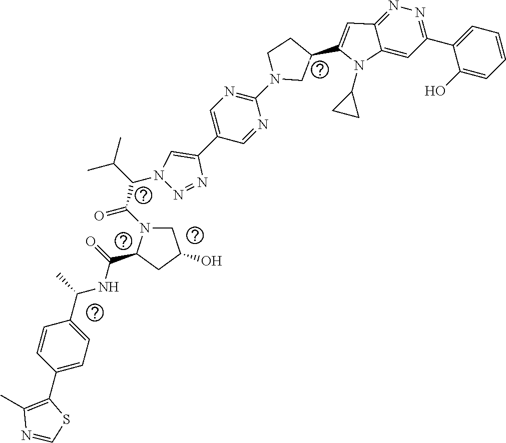 |
| 72 | 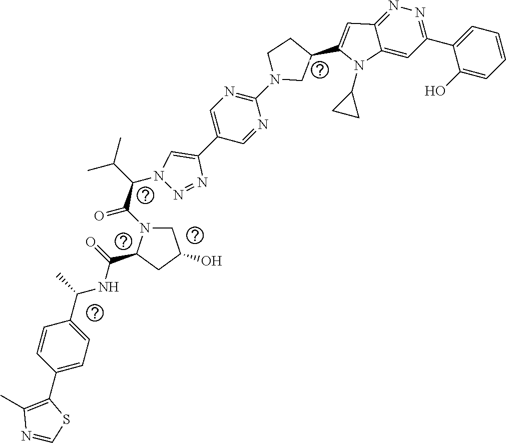 [SMILES: Cc1ncsc1-c1ccc([C@H](C)NC(=O)[C@@H]2C[C@@H](O)CN2C(=O)[C@@H](C(C)C)n2cc(-c3cnc(N4CC[C@@H](c5cc6nnc(-c7ccccc7O)cc6n5C5CC5)C4)nc3)nn2)cc1] |
| 73 | 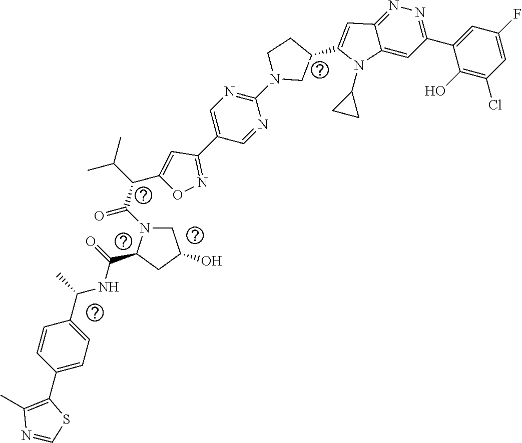 [SMILES: Cc1ncsc1-c1ccc([C@H](C)NC(=O)[C@@H]2C[C@@H](O)CN2C(=O)[C@@H](c2cc(-c3cnc(N4CC[C@H](c5cc6nnc(-c7cc(F)cc(Cl)c7O)cc6n5C5CC5)C4)nc3)no2)C(C)C)cc1] |
| 74 | 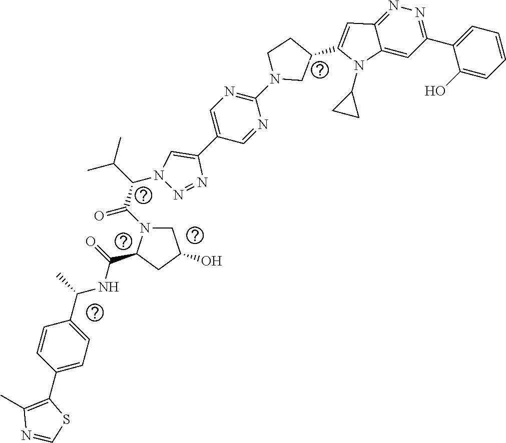 [SMILES: Cc1ncsc1-c1ccc([C@H](C)NC(=O)[C@@H]2C[C@@H](O)CN2C(=O)[C@H](C(C)C)n2cc(-c3cnc(N4CC[C@H](c5cc6nnc(-c7ccccc7O)cc6n5C5CC5)C4)nc3)nn2)cc1] |
| 75 | 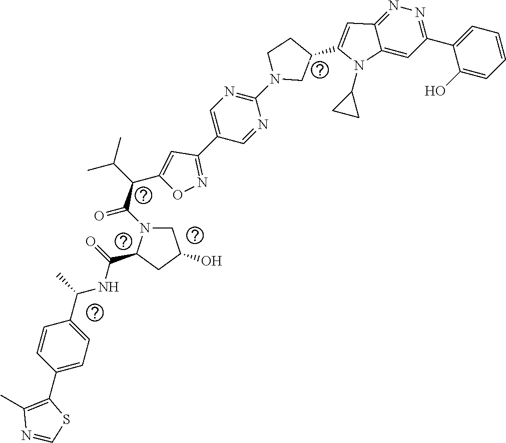 [SMILES: Cc1ncsc1-c1ccc([C@H](C)NC(=O)[C@@H]2C[C@@H](O)CN2C(=O)[C@H](c2cc(-c3cnc(N4CC[C@H](c5cc6nnc(-c7ccccc7O)cc6n5C5CC5)C4)nc3)no2)C(C)C)cc1] |
| 76 | 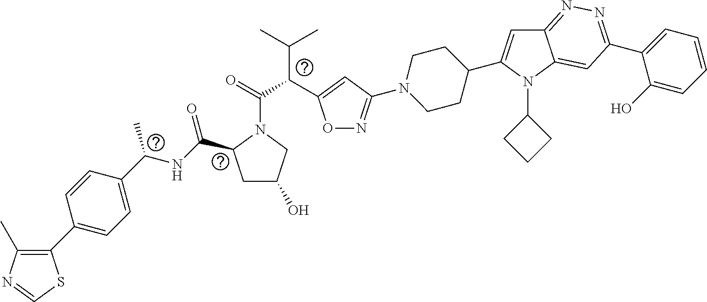 [SMILES: Cc1ncsc1-c1ccc([C@H](C)NC(=O)[C@@H]2C[C@@H](O)CN2C(=O)[C@@H](c2cc(N3CCC(c4cc5nnc(-c6ccccc6O)cc5n4C4CCC4)CC3)no2)C(C)C)cc1] |
| 77 | 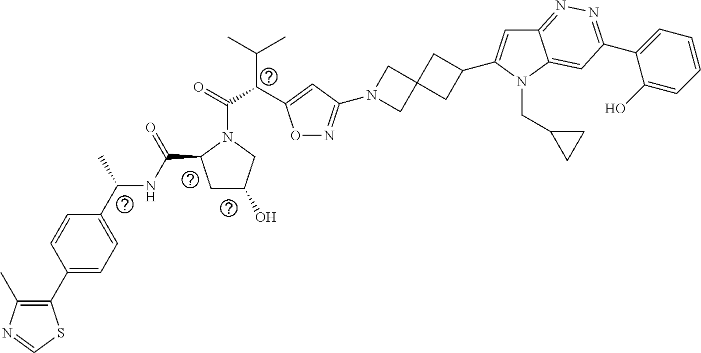 [SMILES: Cc1ncsc1-c1ccc([C@H](C)NC(=O)[C@@H]2C[C@@H](O)CN2C(=O)[C@@H](c2cc(N3CC4(CC(c5cc6nnc(-c7ccccc7O)cc6n5CC5CC5)C4)C3)no2)C(C)C)cc1] |
| 78 | 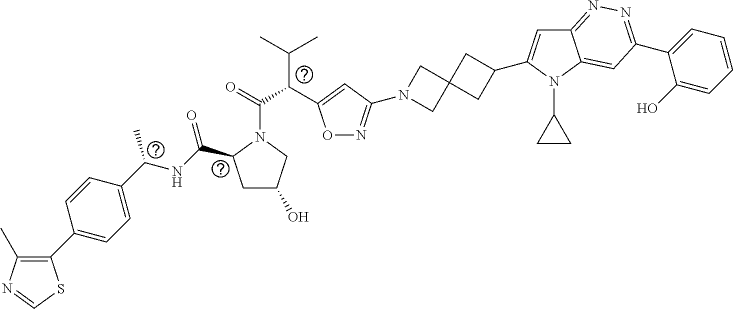 [SMILES: Cc1ncsc1-c1ccc([C@H](C)NC(=O)[C@@H]2C[C@@H](O)CN2C(=O)[C@@H](c2cc(N3CC4(CC(c5cc6nnc(-c7ccccc7O)cc6n5C5CC5)C4)C3)no2)C(C)C)cc1] |
| 79 | 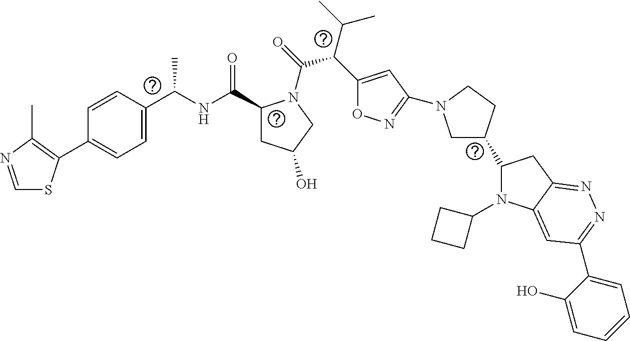 [SMILES: Cc1ncsc1-c1ccc([C@H](C)NC(=O)[C@@H]2C[C@@H](O)CN2C(=O)[C@@H](c2cc(N3CC[C@H](C4Cc5nnc(-c6ccccc6O)cc5N4C4CCC4)C3)no2)C(C)C)cc1] |
| 80 | 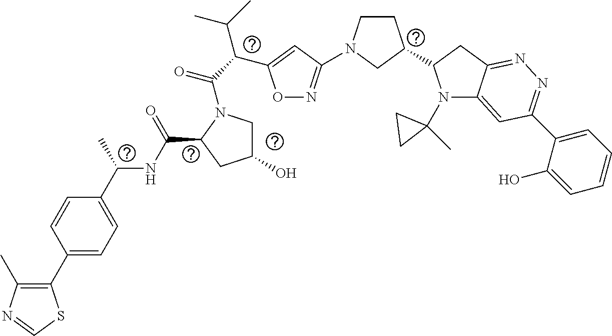 [SMILES: Cc1ncsc1-c1ccc([C@H](C)NC(=O)[C@@H]2C[C@@H](O)CN2C(=O)[C@@H](c2cc(N3CC[C@H](C4Cc5nnc(-c6ccccc6O)cc5N4C4(C)CC4)C3)no2)C(C)C)cc1] |
| 81 | 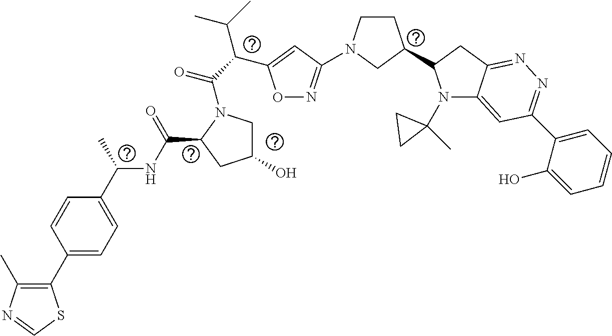 [SMILES: Cc1ncsc1-c1ccc([C@H](C)NC(=O)[C@@H]2C[C@@H](O)CN2C(=O)[C@@H](c2cc(N3CC[C@@H](C4Cc5nnc(-c6ccccc6O)cc5N4C4(C)CC4)C3)no2)C(C)C)cc1] |
| 82 | 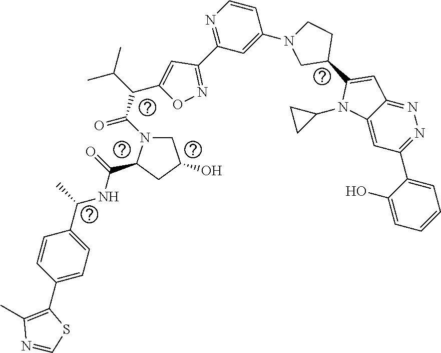 |
| 83 | 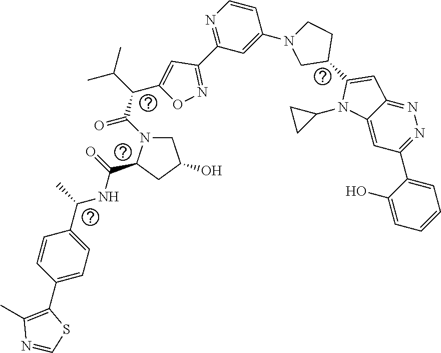 [SMILES: Cc1ncsc1-c1ccc([C@H](C)NC(=O)[C@@H]2C[C@@H](O)CN2C(=O)[C@@H](c2cc(-c3cc(N4CC[C@H](c5cc6nnc(-c7ccccc7O)cc6n5C5CC5)C4)ccn3)no2)C(C)C)cc1] |
| 84 | 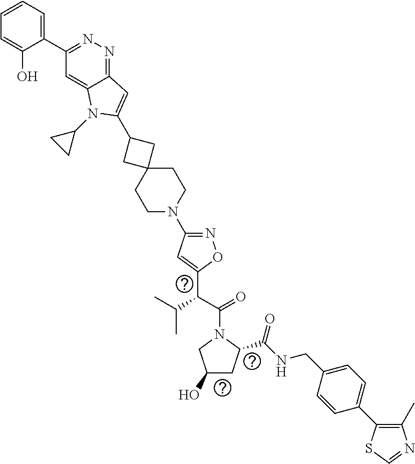 [SMILES: Cc1ncsc1-c1ccc(CNC(=O)[C@@H]2C[C@@H](O)CN2C(=O)[C@@H](c2cc(N3CCC4(CC3)CC(c3cc5nnc(-c6ccccc6O)cc5n3C3CC3)C4)no2)C(C)C)cc1] |
| 85 | 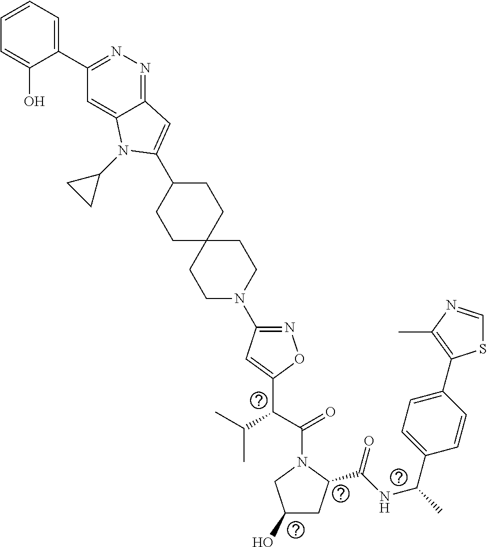 [SMILES: Cc1ncsc1-c1ccc([C@H](C)NC(=O)[C@@H]2C[C@@H](O)CN2C(=O)[C@@H](c2cc(N3CCC4(CCC(c5cc6nnc(-c7ccccc7O)cc6n5C5CC5)CC4)CC3)no2)C(C)C)cc1] |
| 86 | 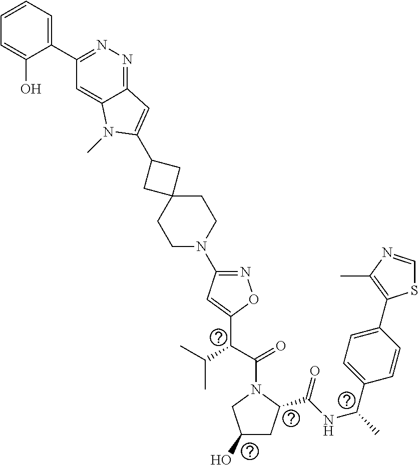 [SMILES: Cc1ncsc1-c1ccc([C@H](C)NC(=O)[C@@H]2C[C@@H](O)CN2C(=O)[C@@H](c2cc(N3CCC4(CC3)CC(c3cc5nnc(-c6ccccc6O)cc5n3C)C4)no2)C(C)C)cc1] |
| 87 | 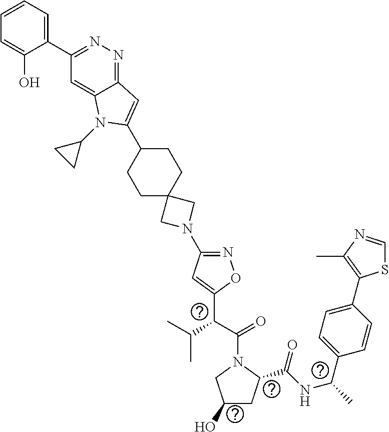 [SMILES: Cc1ncsc1-c1ccc([C@H](C)NC(=O)[C@@H]2C[C@@H](O)CN2C(=O)[C@@H](c2cc(N3CC4(CCC(c5cc6nnc(-c7ccccc7O)cc6n5C5CC5)CC4)C3)no2)C(C)C)cc1] |
| 88 | 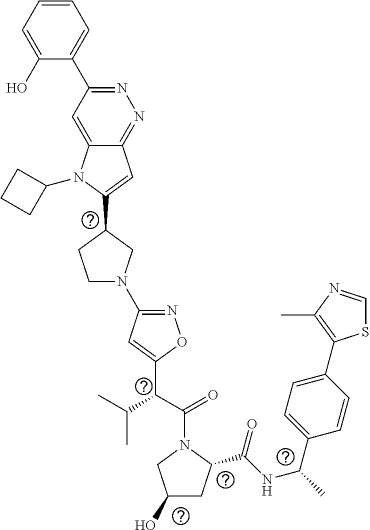 [SMILES: Cc1ncsc1-c1ccc([C@H](C)NC(=O)[C@@H]2C[C@@H](O)CN2C(=O)[C@@H](c2cc(N3CC[C@@H](c4cc5nnc(-c6ccccc6O)cc5n4C4CCC4)C3)no2)C(C)C)cc1] |
| 89 | 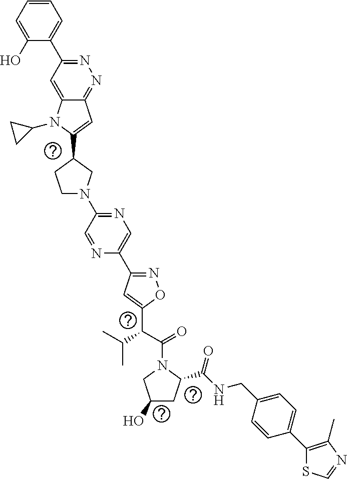 [SMILES: Cc1ncsc1-c1ccc(CNC(=O)[C@@H]2C[C@@H](O)CN2C(=O)[C@@H](c2cc(-c3cnc(N4CC[C@@H](c5cc6nnc(-c7ccccc7O)cc6n5C5CC5)C4)cn3)no2)C(C)C)cc1] |
| 90 | 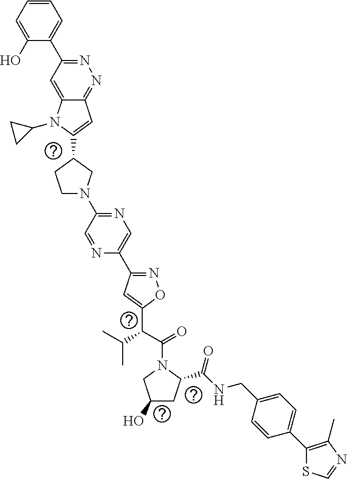 [SMILES: Cc1ncsc1-c1ccc(CNC(=O)[C@@H]2C[C@@H](O)CN2C(=O)[C@@H](c2cc(-c3cnc(N4CC[C@H](c5cc6nnc(-c7ccccc7O)cc6n5C5CC5)C4)cn3)no2)C(C)C)cc1] |
| 91 | 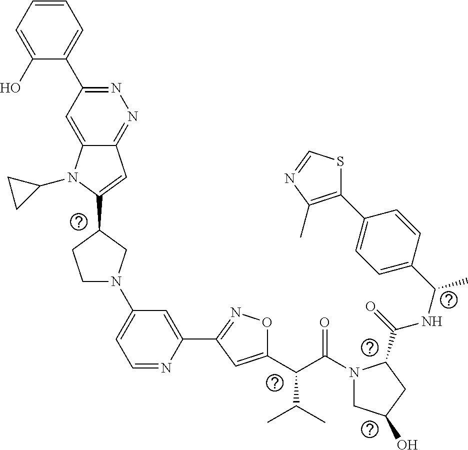 [SMILES: Cc1ncsc1-c1ccc([C@H](C)NC(=O)[C@@H]2C[C@@H](O)CN2C(=O)[C@@H](c2cc(-c3cc(N4CC[C@@H](c5cc6nnc(-c7ccccc7O)cc6n5C5CC5)C4)ccn3)no2)C(C)C)cc1] |
| 92 | 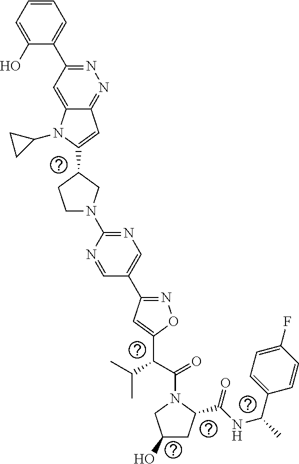 [SMILES: CC(C)[C@@H](C(=O)N1C[C@H](O)C[C@H]1C(=O)N[C@@H](C)c1ccc(F)cc1)c1cc(-c2cnc(N3CC[C@H](c4cc5nnc(-c6ccccc6O)cc5n4C4CC4)C3)nc2)no1] |
| 93 | 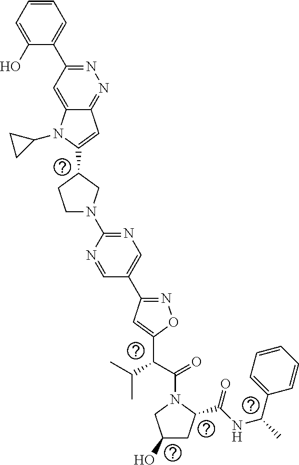 [SMILES: CC(C)[C@@H](C(=O)N1C[C@H](O)C[C@H]1C(=O)N[C@@H](C)c1ccccc1)c1cc(-c2cnc(N3CC[C@H](c4cc5nnc(-c6ccccc6O)cc5n4C4CC4)C3)nc2)no1] |
| 94 | 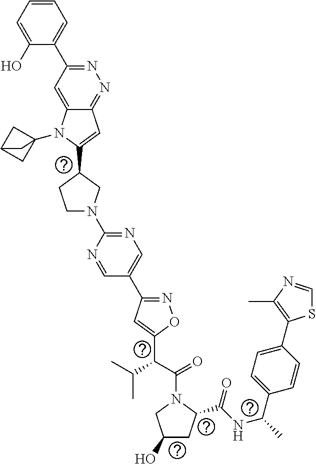 [SMILES: Cc1ncsc1-c1ccc([C@H](C)NC(=O)[C@@H]2C[C@@H](O)CN2C(=O)[C@@H](c2cc(-c3cnc(N4CC[C@@H](c5cc6nnc(-c7ccccc7O)cc6n5C56CC(C5)C6)C4)nc3)no2)C(C)C)cc1] |
| 95 | 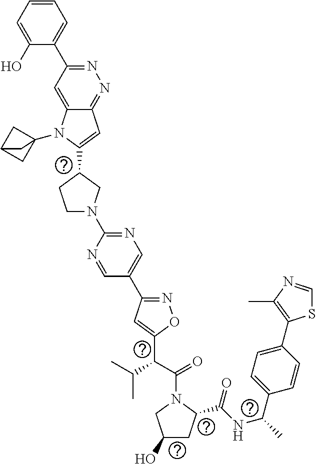 [SMILES: Cc1ncsc1-c1ccc([C@H](C)NC(=O)[C@@H]2C[C@@H](O)CN2C(=O)[C@@H](c2cc(-c3cnc(N4CC[C@H](c5cc6nnc(-c7ccccc7O)cc6n5C56CC(C5)C6)C4)nc3)no2)C(C)C)cc1] |
| 96 | 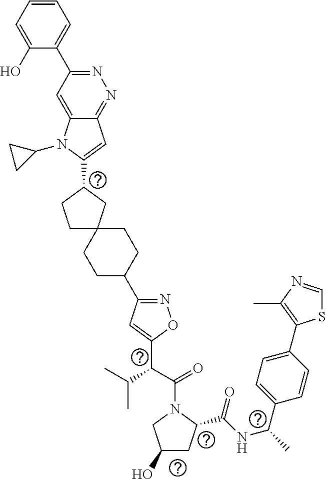 [SMILES: Cc1ncsc1-c1ccc([C@H](C)NC(=O)[C@@H]2C[C@@H](O)CN2C(=O)[C@@H](c2cc(C3CCC4(CC3)CC[C@H](c3cc5nnc(-c6ccccc6O)cc5n3C3CC3)C4)no2)C(C)C)cc1] |
| 97 | 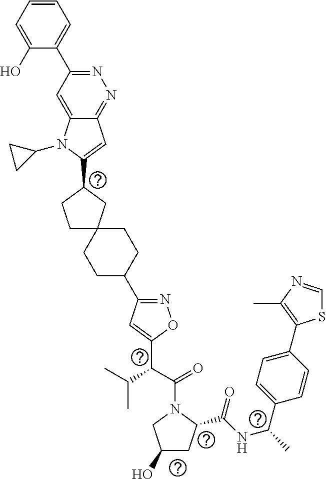 [SMILES: Cc1ncsc1-c1ccc([C@H](C)NC(=O)[C@@H]2C[C@@H](O)CN2C(=O)[C@@H](c2cc(C3CCC4(CC3)CC[C@@H](c3cc5nnc(-c6ccccc6O)cc5n3C3CC3)C4)no2)C(C)C)cc1] |
| 98 | 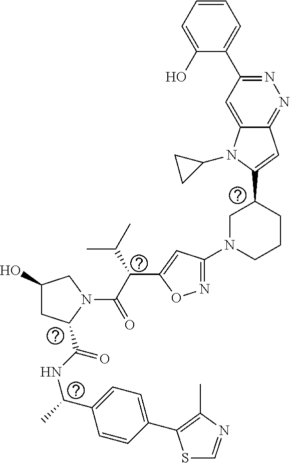 [SMILES: Cc1ncsc1-c1ccc([C@H](C)NC(=O)[C@@H]2C[C@@H](O)CN2C(=O)[C@@H](c2cc(N3CCC[C@H](c4cc5nnc(-c6ccccc6O)cc5n4C4CC4)C3)no2)C(C)C)cc1] |
| 99 | 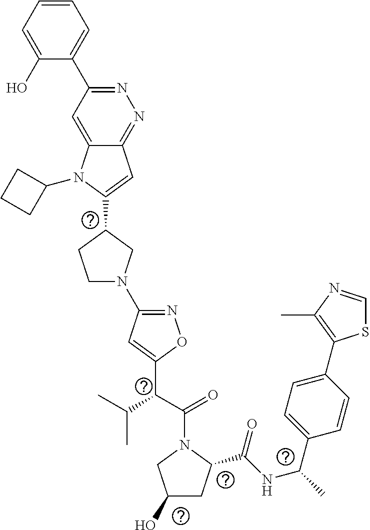 [SMILES: Cc1ncsc1-c1ccc([C@H](C)NC(=O)[C@@H]2C[C@@H](O)CN2C(=O)[C@@H](c2cc(N3CC[C@H](c4cc5nnc(-c6ccccc6O)cc5n4C4CCC4)C3)no2)C(C)C)cc1] |
| 100 | 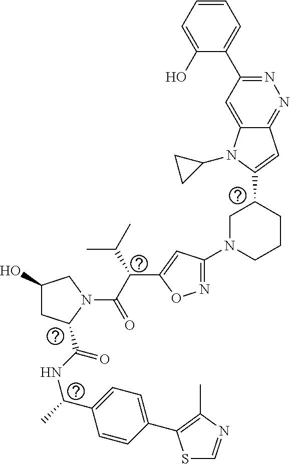 [SMILES: Cc1ncsc1-c1ccc([C@H](C)NC(=O)[C@@H]2C[C@@H](O)CN2C(=O)[C@@H](c2cc(N3CCC[C@@H](c4cc5nnc(-c6ccccc6O)cc5n4C4CC4)C3)no2)C(C)C)cc1] |
| 101 | 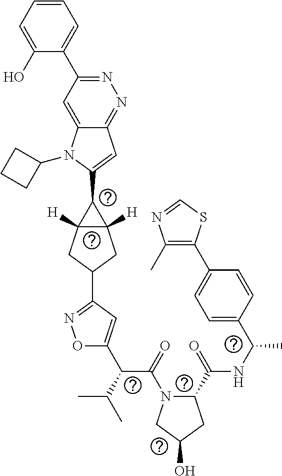 [SMILES: Cc1ncsc1-c1ccc([C@H](C)NC(=O)[C@@H]2C[C@@H](O)CN2C(=O)[C@@H](c2cc(C3C[C@@H]4[C@H](C3)[C@H]4c3cc4nnc(-c5ccccc5O)cc4n3C3CCC3)no2)C(C)C)cc1] |
| 102 | 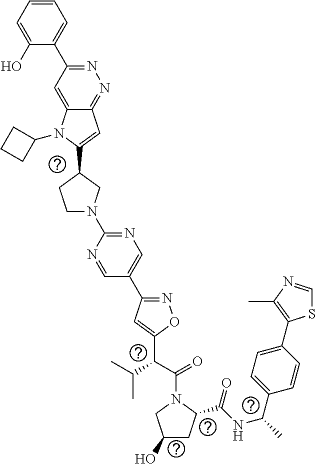 [SMILES: Cc1ncsc1-c1ccc([C@H](C)NC(=O)[C@@H]2C[C@@H](O)CN2C(=O)[C@@H](c2cc(-c3cnc(N4CC[C@@H](c5cc6nnc(-c7ccccc7O)cc6n5C5CCC5)C4)nc3)no2)C(C)C)cc1] |
| 103 | 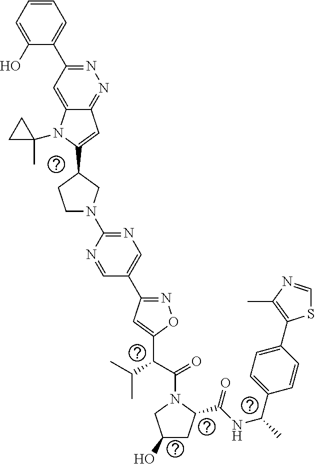 [SMILES: Cc1ncsc1-c1ccc([C@H](C)NC(=O)[C@@H]2C[C@@H](O)CN2C(=O)[C@@H](c2cc(-c3cnc(N4CC[C@@H](c5cc6nnc(-c7ccccc7O)cc6n5C5(C)CC5)C4)nc3)no2)C(C)C)cc1] |
| 104 | 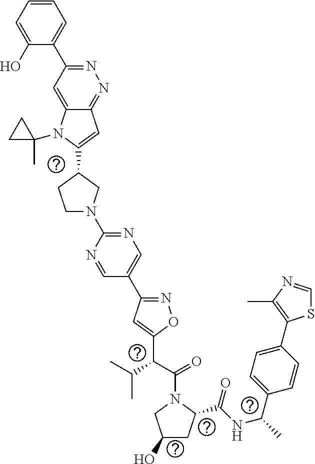 [SMILES: Cc1ncsc1-c1ccc([C@H](C)NC(=O)[C@@H]2C[C@@H](O)CN2C(=O)[C@@H](c2cc(-c3cnc(N4CC[C@H](c5cc6nnc(-c7ccccc7O)cc6n5C5(C)CC5)C4)nc3)no2)C(C)C)cc1] |
| 105 | 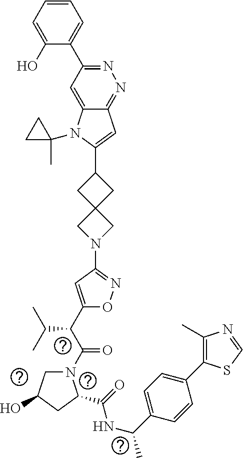 [SMILES: Cc1ncsc1-c1ccc([C@H](C)NC(=O)[C@@H]2C[C@@H](O)CN2C(=O)[C@@H](c2cc(N3CC4(CC(c5cc6nnc(-c7ccccc7O)cc6n5C5(C)CC5)C4)C3)no2)C(C)C)cc1] |
| 106 | 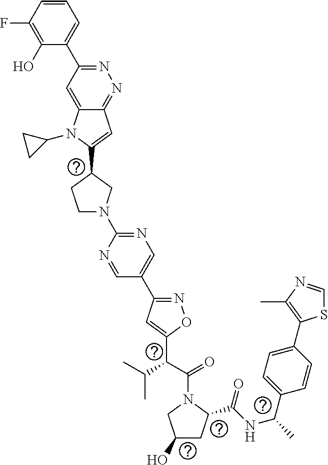 [SMILES: Cc1ncsc1-c1ccc([C@H](C)NC(=O)[C@@H]2C[C@@H](O)CN2C(=O)[C@@H](c2cc(-c3cnc(N4CC[C@@H](c5cc6nnc(-c7cccc(F)c7O)cc6n5C5CC5)C4)nc3)no2)C(C)C)cc1] |
| 107 | 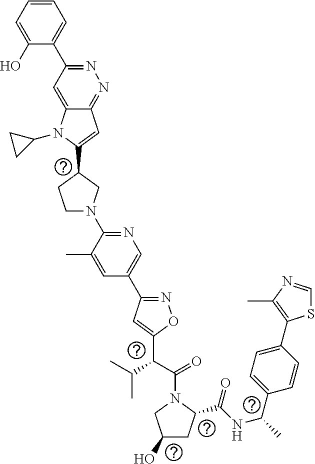 [SMILES: Cc1cc(-c2cc([C@H](C(=O)N3C[C@H](O)C[C@H]3C(=O)N[C@@H](C)c3ccc(-c4scnc4C)cc3)C(C)C)on2)cnc1N1CC[C@@H](c2cc3nnc(-c4ccccc4O)cc3n2C2CC2)C1] |
| 108 | 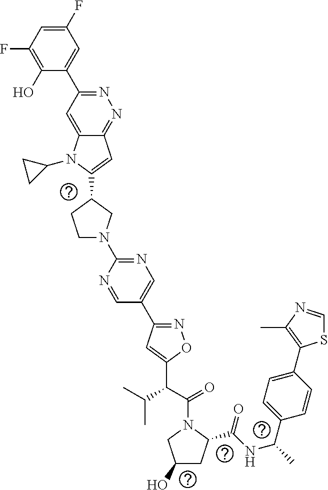 [SMILES: Cc1ncsc1-c1ccc([C@H](C)NC(=O)[C@@H]2C[C@@H](O)CN2C(=O)[C@@H](c2cc(-c3cnc(N4CC[C@H](c5cc6nnc(-c7cc(F)cc(F)c7O)cc6n5C5CC5)C4)nc3)no2)C(C)C)cc1] |
| 109 | 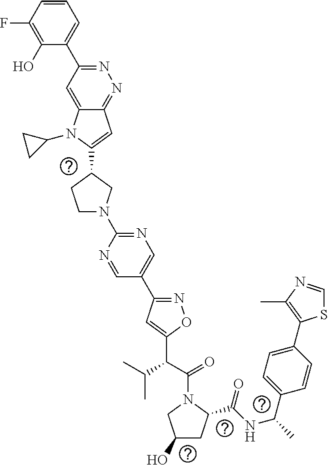 [SMILES: Cc1ncsc1-c1ccc([C@H](C)NC(=O)[C@@H]2C[C@@H](O)CN2C(=O)[C@@H](c2cc(-c3cnc(N4CC[C@H](c5cc6nnc(-c7cccc(F)c7O)cc6n5C5CC5)C4)nc3)no2)C(C)C)cc1] |
| 110 | 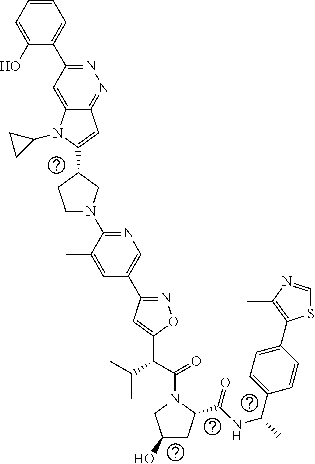 [SMILES: Cc1cc(-c2cc([C@H](C(=O)N3C[C@H](O)C[C@H]3C(=O)N[C@@H](C)c3ccc(-c4scnc4C)cc3)C(C)C)on2)cnc1N1CC[C@H](c2cc3nnc(-c4ccccc4O)cc3n2C2CC2)C1] |
| 111 | 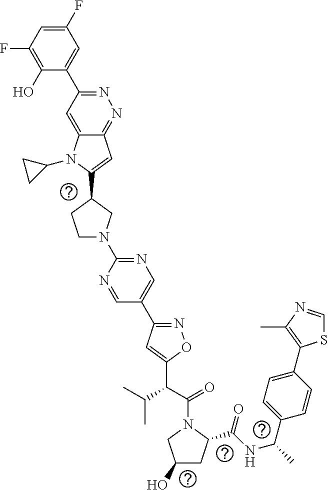 [SMILES: Cc1ncsc1-c1ccc([C@H](C)NC(=O)[C@@H]2C[C@@H](O)CN2C(=O)[C@@H](c2cc(-c3cnc(N4CC[C@@H](c5cc6nnc(-c7cc(F)cc(F)c7O)cc6n5C5CC5)C4)nc3)no2)C(C)C)cc1] |
| 112 | 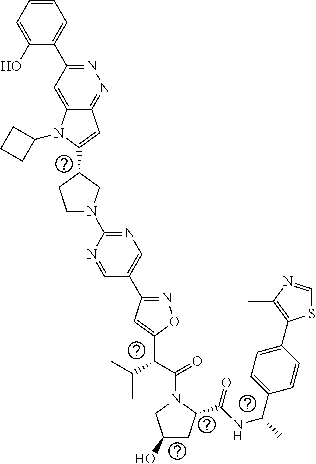 [SMILES: Cc1ncsc1-c1ccc([C@H](C)NC(=O)[C@@H]2C[C@@H](O)CN2C(=O)[C@@H](c2cc(-c3cnc(N4CC[C@H](c5cc6nnc(-c7ccccc7O)cc6n5C5CCC5)C4)nc3)no2)C(C)C)cc1] |
| 113 | 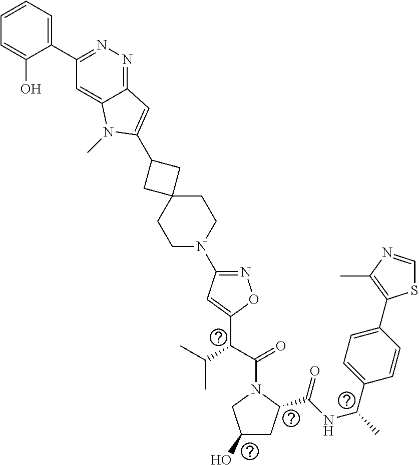 [SMILES: Cc1ncsc1-c1ccc([C@H](C)NC(=O)[C@@H]2C[C@@H](O)CN2C(=O)[C@@H](c2cc(N3CCC4(CC3)CC(c3cc5nnc(-c6ccccc6O)cc5n3C)C4)no2)C(C)C)cc1] |
| 114 | 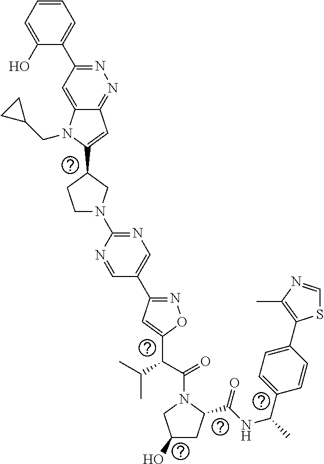 [SMILES: Cc1ncsc1-c1ccc([C@H](C)NC(=O)[C@@H]2C[C@@H](O)CN2C(=O)[C@@H](c2cc(-c3cnc(N4CC[C@@H](c5cc6nnc(-c7ccccc7O)cc6n5CC5CC5)C4)nc3)no2)C(C)C)cc1] |
| 115 | 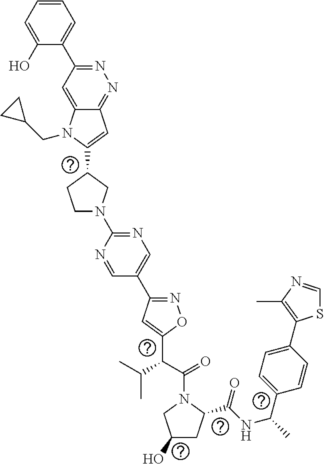 [SMILES: Cc1ncsc1-c1ccc([C@H](C)NC(=O)[C@@H]2C[C@@H](O)CN2C(=O)[C@@H](c2cc(-c3cnc(N4CC[C@H](c5cc6nnc(-c7ccccc7O)cc6n5CC5CC5)C4)nc3)no2)C(C)C)cc1] |
| 116 | 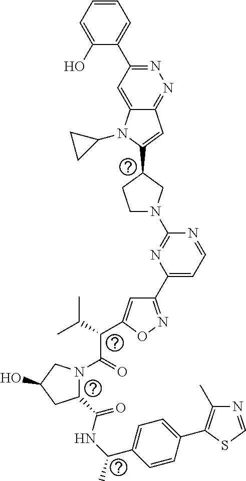 |
| 117 | 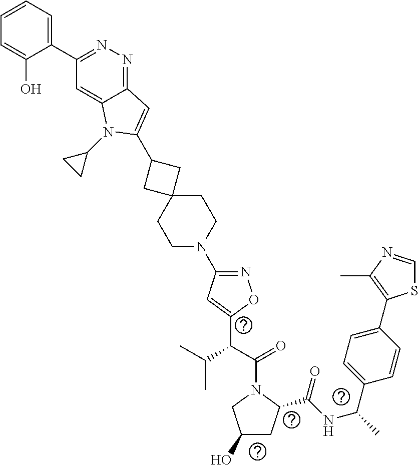 [SMILES: Cc1ncsc1-c1ccc([C@H](C)NC(=O)[C@@H]2C[C@@H](O)CN2C(=O)[C@@H](c2cc(N3CCC4(CC3)CC(c3cc5nnc(-c6ccccc6O)cc5n3C3CC3)C4)no2)C(C)C)cc1] |
| 118 | 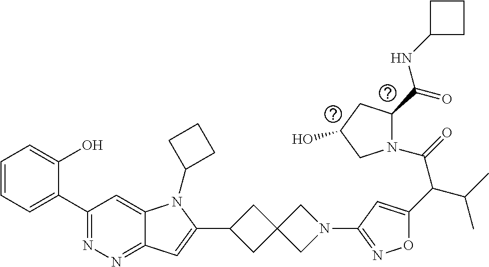 [SMILES: CC(C)C(C(=O)N1C[C@H](O)C[C@H]1C(=O)NC1CCC1)c1cc(N2CC3(CC(c4cc5nnc(-c6ccccc6O)cc5n4C4CCC4)C3)C2)no1] |
| 119 | 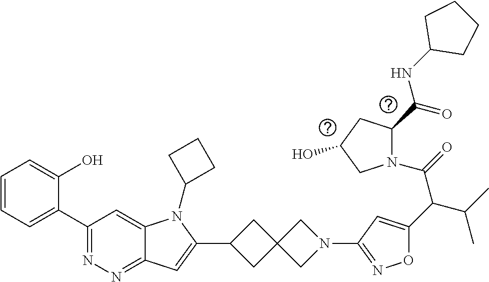 [SMILES: CC(C)C(C(=O)N1C[C@H](O)C[C@H]1C(=O)NC1CCCC1)c1cc(N2CC3(CC(c4cc5nnc(-c6ccccc6O)cc5n4C4CCC4)C3)C2)no1] |
| 120 | 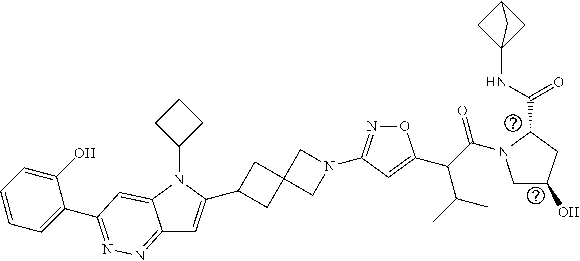 [SMILES: CC(C)C(C(=O)N1C[C@H](O)C[C@H]1C(=O)NC12CC(C1)C2)c1cc(N2CC3(CC(c4cc5nnc(-c6ccccc6O)cc5n4C4CCC4)C3)C2)no1] |
| 121 | 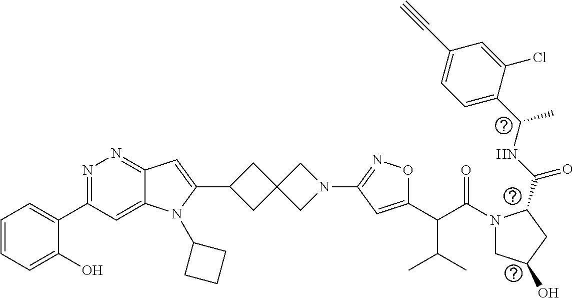 [SMILES: C#Cc1ccc([C@H](C)NC(=O)[C@@H]2C[C@@H](O)CN2C(=O)C(c2cc(N3CC4(CC(c5cc6nnc(-c7ccccc7O)cc6n5C5CCC5)C4)C3)no2)C(C)C)c(Cl)c1] |

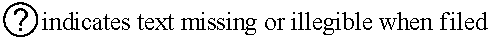

In some embodiments, the compound has a ratio of BRG1 IC₅₀ to BRM IC₅₀ of at least 5. In some embodiments, the compound has a ratio of BRG1 IC₅₀ to BRM IC₅₀ of at least 7. In some embodiments, the compound has a ratio of BRG1 IC₅₀ to BRM IC₅₀ of at least 10. In some embodiments, the compound has a ratio of BRG1 IC₅₀ to BRM IC₅₀ of at least 15. In some embodiments, the compound has a ratio of BRG1 IC₅₀ to BRM IC₅₀ of at least 20. In some embodiments, the compound has a ratio of BRG1 IC₅₀ to BRM IC₅₀ of at least 25. In some embodiments, the compound has a ratio of BRG1 IC₅₀ to BRM IC₅₀ of at least 30.

In an aspect, the invention features a pharmaceutical composition comprising any of the foregoing compounds and a pharmaceutically acceptable excipient.

In another aspect, the invention features a method of decreasing the activity of a BAF complex in a cell, the method involving contacting the cell with an effective amount of any of the foregoing compounds or a pharmaceutical composition thereof.

In some embodiments, the cell is a cancer cell.

In another aspect, the invention features a method of treating a BAF complex-related disorder in a subject in need thereof, the method involving administering to the subject an effective amount of any of the foregoing compounds (e.g., a BRM/BRG1 dual inhibitor compound or a BRM-selective compound) or a pharmaceutical composition thereof.

In some embodiments, the BAF complex-related disorder is cancer or a viral infection.

In a further aspect, the invention features a method of inhibiting BRM, the method involving contacting a cell with an effective amount of any of the foregoing compounds (e.g., a BRM/BRG1 dual inhibitor compound or a BRM-selective compound) or a pharmaceutical composition thereof.

In some embodiments, the cell is a cancer cell.

In another aspect, the invention features a method of inhibiting BRG1, the method involving contacting the cell with an effective amount of any of the foregoing compounds or a pharmaceutical composition thereof.

In some embodiments, the cell is a cancer cell.

In a further aspect, the invention features a method of inhibiting BRM and BRG1, the method involving contacting the cell with an effective amount of any of the foregoing compounds or a pharmaceutical composition thereof.

In some embodiments, the cell is a cancer cell.

In another aspect, the invention features a method of treating a disorder related to a BRG1 loss of function mutation in a subject in need thereof, the method involving administering to the subject an effective amount of any of the foregoing compounds (e.g., a BRM/BRG1 dual inhibitor compound or a BRM-selective compound) or a pharmaceutical composition thereof.

In some embodiments, the disorder related to a BRG1 loss of function mutation is cancer.

In other embodiments, the subject is determined to have a BRG1 loss of function disorder, for example, is determined to have a BRG1 loss of function cancer (for example, the cancer has been determined to include cancer cells with loss of BRG1 function).

In another aspect, the invention features a method of inducing apoptosis in a cell, the method involving contacting the cell with an effective amount of any of the foregoing compounds (e.g., a BRM/BRG1 dual inhibitor compound or a BRM-selective compound) or a pharmaceutical composition thereof.

In some embodiments, the cell is a cancer cell.

In a further aspect, the invention features a method of treating cancer in a subject in need thereof, the method including administering to the subject an effective amount of any of the foregoing compounds (e.g., a BRM/BRG1 dual inhibitor compound or a BRM-selective compound) or a pharmaceutical composition thereof.

In some embodiments of any of the foregoing methods, the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

In some embodiments of any of the foregoing methods, the cancer is non-small cell lung cancer, colorectal cancer, bladder cancer, cancer of unknown primary, glioma, breast cancer, melanoma, non-melanoma skin cancer, endometrial cancer, or penile cancer.

In some embodiments, the cancer is non-small cell lung cancer. In some embodiments, the cancer is soft tissue sarcoma.

In some embodiments of any of the foregoing methods, the cancer is a drug resistant cancer or has failed to respond to a prior therapy (e.g., vemurafenib, dacarbazine, a CTLA4 inhibitor, a PD1 inhibitor, interferon therapy, a BRAF inhibitor, a MEK inhibitor, radiotherapy, temozolomide, irinotecan, a CAR-T therapy, Herceptin®, Perjeta®, tamoxifen, Xeloda®, docetaxol, platinum agents such as carboplatin, taxanes such as paclitaxel and docetaxel, ALK inhibitors, MET inhibitors, Alimta®, Abraxane®, Adriamycin®, gemcitabine, Avastin®, Halaven®, neratinib, a PARP inhibitor, ARN810, an mTOR inhibitor, topotecan, Gemzar®, a VEGFR2 inhibitor, a folate receptor antagonist, demcizumab, fosbretabulin, or a PDL1 inhibitor).

In some embodiments of any of the foregoing methods, the cancer has or has been determined to have BRG1 mutations. In some embodiments of any of the foregoing methods, the BRG1 mutations are homozygous. In some embodiments of any of the foregoing methods, the cancer does not have, or has been determined not to have, an epidermal growth factor receptor (EGFR) mutation. In some embodiments of any of the foregoing methods, the cancer does not have, or has been determined not to have, an anaplastic lymphoma kinase (ALK) driver mutation. In some embodiments of any of the foregoing methods, the cancer has, or has been determined to have, a KRAS mutation. In some embodiments of any of the foregoing methods, the BRG1 mutation is in the ATPase catalytic domain of the protein. In some embodiments of any of the foregoing methods, the BRG1 mutation is a deletion at the C-terminus of BRG1.

In another aspect, the disclosure provides a method treating a disorder related to BAF (e.g., cancer or viral infections) in a subject in need thereof. This method includes contacting a cell with an effective amount of any of the foregoing compounds (e.g., a BRM/BRG1 dual inhibitor compound or a BRM-selective compound), or pharmaceutically acceptable salts thereof, or any of the foregoing pharmaceutical compositions. In some embodiments, the disorder is a viral infection is an infection with a virus of the Retroviridae family such as the lentiviruses (e.g., Human immunodeficiency virus (HIV) and deltaretroviruses (e.g., human T cell leukemia virus I (HTLV-I), human T cell leukemia virus II (HTLV-II)), Hepadnaviridae family (e.g., hepatitis B virus (HBV)), Flaviviridae family (e.g., hepatitis C virus (HCV)), Adenoviridae family (e.g., Human Adenovirus), Herpesviridae family (e.g., Human cytomegalovirus (HCMV), Epstein-Barr virus, herpes simplex virus 1 (HSV-1), herpes simplex virus 2 (HSV-2), human herpesvirus 6 (HHV-6), Herpesvirus K*, CMV, varicella-zoster virus), Papillomaviridae family (e.g., Human Papillomavirus (HPV, HPV E1)), Parvoviridae family (e.g., Parvovirus B19), Polyomaviridae family (e.g., JC virus and BK virus), Paramyxoviridae family (e.g., Measles virus), Togaviridae family (e.g., Rubella virus). In some embodiments, the disorder is Coffin Siris, Neurofibromatosis (e.g., NF-1, NF-2, or Schwannomatosis), or Multiple Meningioma.

In another aspect, the disclosure provides a method for treating a viral infection in a subject in need thereof. This method includes administering to the subject an effective amount of any of the foregoing compounds (e.g., a BRM/BRG1 dual inhibitor compound or a BRM-selective compound), or pharmaceutically acceptable salts thereof, or any of the foregoing pharmaceutical compositions. In some embodiments, the viral infection is an infection with a virus of the Retroviridae family such as the lentiviruses (e.g., Human immunodeficiency virus (HIV) and deltaretroviruses (e.g., human T cell leukemia virus I (HTLV-I), human T cell leukemia virus II (HTLV-II)), Hepadnaviridae family (e.g., hepatitis B virus (HBV)), Flaviviridae family (e.g., hepatitis C virus (HCV)), Adenoviridae family (e.g., Human Adenovirus), Herpesviridae family (e.g., Human cytomegalovirus (HCMV), Epstein-Barr virus, herpes simplex virus 1 (HSV-1), herpes simplex virus 2 (HSV-2), human herpesvirus 6 (HHV-6), Herpesvirus K*, CMV, varicella-zoster virus), Papillomaviridae family (e.g., Human Papillomavirus (HPV, HPV E1)), Parvoviridae family (e.g., Parvovirus B19), Polyomaviridae family (e.g., JC virus and BK virus), Paramyxoviridae family (e.g., Measles virus), or Togaviridae family (e.g., Rubella virus).

In some embodiments of any of the foregoing aspects, the compound is a BRM-selective compound. In some embodiments, the BRM-selective compound inhibits the level and/or activity of BRM at least 10-fold greater than the compound inhibits the level and/or activity of BRG1 and/or the compound binds to BRM at least 10-fold greater than the compound binds to BRG1. For example, in some embodiments, a BRM-selective compound has an IC₅₀ or IP₅₀ that is at least 10-fold lower than the IC₅₀ or IP₅₀ against BRG1. In some embodiments of any of the foregoing aspects, the compound is a BRM/BRG1 dual inhibitor compound. In some embodiments, the BRM/BRG1 dual inhibitor compound has similar activity against both BRM and BRG1 (e.g., the activity of the compound against BRM and BRG1 with within 10-fold (e.g., less than 5-fold, less than 2-fold). In some embodiments, the activity of the BRM/BRG1 dual inhibitor compound is greater against BRM. In some embodiments, the activity of the BRM/BRG1 dual inhibitor compound is greater against BRG1. For example, in some embodiments, a BRM/BRG1 dual inhibitor compound has an IC₅₀ or IP₅₀ against BRM that is within 10-fold of the IC₅₀ or IP₅₀ against BRG1.

In another aspect, the invention features a method of treating melanoma, prostate cancer, breast cancer, bone cancer, renal cell carcinoma, or a hematologic cancer in a subject in need thereof, the method including administering to the subject an effective amount of any of the foregoing compounds or pharmaceutical compositions thereof.

In another aspect, the invention features a method of reducing tumor growth of melanoma, prostate cancer, breast cancer, bone cancer, renal cell carcinoma, or a hematologic cancer in a subject in need thereof, the method including administering to the subject an effective amount of any of the foregoing compounds or pharmaceutical compositions thereof.

In another aspect, the invention features a method of suppressing metastatic progression of melanoma, prostate cancer, breast cancer, bone cancer, renal cell carcinoma, or a hematologic cancer in a subject, the method including administering an effective amount of any of the foregoing compounds or pharmaceutical compositions thereof.

In another aspect, the invention features a method of suppressing metastatic colonization of melanoma, prostate cancer, breast cancer, bone cancer, renal cell carcinoma, or a hematologic cancer in a subject, the method including administering an effective amount of any of the foregoing compounds or pharmaceutical compositions thereof.

In another aspect, the invention features a method of reducing the level and/or activity of BRG1 and/or BRM in a melanoma, prostate cancer, breast cancer, bone cancer, renal cell carcinoma, or hematologic cancer cell, the method including contacting the cell with an effective amount of any of the foregoing compounds or pharmaceutical compositions thereof.

In some embodiments of any of the above aspects, the melanoma, prostate cancer, breast cancer, bone cancer, renal cell carcinoma, or hematologic cell is in a subject.

In some embodiments of any of the above aspects, the effective amount of the compound reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the compound that reduces the level and/or activity of BRG1 by at least 50% (e.g., 55%, 60%, 65%, 70%, 75%, 80%, 85%, 90%, or 95%) as compared to a reference. In some embodiments, the effective amount of the compound that reduces the level and/or activity of BRG1 by at least 90% (e.g., 91%, 92%, 93%, 94%, 95%, 96%, 97%, 98%, or 99%).

In some embodiments, the effective amount of the compound reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the compound that reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments of any of the above aspects, the effective amount of the compound reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the compound that reduces the level and/or activity of BRM by at least 50% (e.g., 55%, 60%, 65%, 70%, 75%, 80%, 85%, 90%, or 95%) as compared to a reference. In some embodiments, the effective amount of the compound that reduces the level and/or activity of BRM by at least 90% (e.g., 91%, 92%, 93%, 94%, 95%, 96%, 97%, 98%, or 99%).

In some embodiments, the effective amount of the compound reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the compound that reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments, the subject has cancer. In some embodiments, the cancer expresses BRG1 and/or BRM protein and/or the cell or subject has been identified as expressing BRG1 and/or BRM. In some embodiments, the cancer expresses BRG1 protein and/or the cell or subject has been identified as expressing BRG1. In some embodiments, the cancer expresses BRM protein and/or the cell or subject has been identified as expressing BRM. In some embodiments, the cancer is melanoma (e.g., uveal melanoma, mucosal melanoma, or cutaneous melanoma). In some embodiments, the cancer is prostate cancer. In some embodiments, the cancer is a hematologic cancer, e.g., multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia (e.g., T-cell acute lymphoblastic leukemia or B-cell acute lymphoblastic leukemia), diffuse large cell lymphoma, or non-Hodgkin's lymphoma. In some embodiments, the cancer is breast cancer (e.g., an ER positive breast cancer, an ER negative breast cancer, triple positive breast cancer, or triple negative breast cancer). In some embodiments, the cancer is a bone cancer (e.g., Ewing's sarcoma). In some embodiments, the cancer is a renal cell carcinoma (e.g., a Microphthalmia Transcription Factor (MITF) family translocation renal cell carcinoma (tRCC)). In some embodiments, the cancer is metastatic (e.g., the cancer has spread to the liver). The metastatic cancer can include cells exhibiting migration and/or invasion of migrating cells and/or include cells exhibiting endothelial recruitment and/or angiogenesis. In other embodiments, the migrating cancer is a cell migration cancer. In still other embodiments, the cell migration cancer is a non-metastatic cell migration cancer. The metastatic cancer can be a cancer spread via seeding the surface of the peritoneal, pleural, pericardial, or subarachnoid spaces. Alternatively, the metastatic cancer can be a cancer spread via the lymphatic system, or a cancer spread hematogenously. In some embodiments, the effective amount of an agent that reduces the level and/or activity of BRG1 and/or BRM is an amount effective to inhibit metastatic colonization of the cancer to the liver.

In some embodiments the cancer harbors a mutation in GNAQ. In some embodiments the cancer harbors a mutation in GNA11. In some embodiments the cancer harbors a mutation in PLCB4. In some embodiments the cancer harbors a mutation in CYSLTR2. In some embodiments the cancer harbors a mutation in BAP1. In some embodiments the cancer harbors a mutation in SF3B1. In some embodiments the cancer harbors a mutation in EIF1AX. In some embodiments the cancer harbors a TFE3 translocation. In some embodiments the cancer harbors a TFEB translocation. In some embodiments the cancer harbors a MITF translocation. In some embodiments the cancer harbors an EZH2 mutation. In some embodiments the cancer harbors a SUZ12 mutation. In some embodiments the cancer harbors an EED mutation.

In some embodiments, the method further includes administering to the subject or contacting the cell with an anticancer therapy, e.g., a chemotherapeutic or cytotoxic agent, immunotherapy, surgery, radiotherapy, thermotherapy, or photocoagulation. In some embodiments, the anticancer therapy is a chemotherapeutic or cytotoxic agent, e.g., an antimetabolite, antimitotic, antitumor antibiotic, asparagine-specific enzyme, bisphosphonates, antineoplastic, alkylating agent, DNA-Repair enzyme inhibitor, histone deacetylase inhibitor, corticosteroid, demethylating agent, immunomodulatory, janus-associated kinase inhibitor, phosphinositide 3-kinase inhibitor, proteasome inhibitor, or tyrosine kinase inhibitor.

In some embodiments, the compound of the invention is used in combination with another anti-cancer therapy used for the treatment of uveal melanoma such as surgery, a MEK inhibitor, and/or a PKC inhibitor. For example, in some embodiments, the method further comprises performing surgery prior to, subsequent to, or at the same time as administration of the compound of the invention. In some embodiments, the method further comprises administration of a MEK inhibitor and/or a PKC inhibitor prior to, subsequent to, or at the same time as administration of the compound of the invention.

In some embodiments, the anticancer therapy and the compound of the invention are administered within 28 days of each other and each in an amount that together are effective to treat the subject.

In some embodiments, the subject or cancer has and/or has been identified as having a BRG1 loss of function mutation.

In some embodiments, the cancer is resistant to one or more chemotherapeutic or cytotoxic agents (e.g., the cancer has been determined to be resistant to chemotherapeutic or cytotoxic agents such as by genetic markers, or is likely to be resistant, to chemotherapeutic or cytotoxic agents such as a cancer that has failed to respond to a chemotherapeutic or cytotoxic agent). In some embodiments, the cancer has failed to respond to one or more chemotherapeutic agents. In some embodiments, the cancer is resistant or has failed to respond to dacarbazine, temozolomide, cisplatin, treosulfan, fotemustine, IMCgp100, a CTLA-4 inhibitor (e.g., ipilimumab), a PD-1 inhibitor (e.g., Nivolumab or pembrolizumab), a PD-L1 inhibitor (e.g., atezolizumab, avelumab, or durvalumab), a mitogen-activated protein kinase (MEK) inhibitor (e.g., selumetinib, binimetinib, or tametinib), and/or a protein kinase C (PKC) inhibitor (e.g., sotrastaurin or IDE196).

In some embodiments, the cancer is resistant to or failed to respond to a previously administered therapeutic used for the treatment of uveal melanoma such as a MEK inhibitor or PKC inhibitor. For example, in some embodiments, the cancer is resistant to or failed to respond to a mitogen-activated protein kinase (MEK) inhibitor (e.g., selumetinib, binimetinib, or tametinib), and/or a protein kinase C (PKC) inhibitor (e.g., sotrastaurin or IDE196).

In an aspect, the invention provides the use of any of the foregoing compounds (e.g., a BRM/BRG1 dual inhibitor compound or a BRM-selective compound), or pharmaceutically acceptable salts thereof, or any of the foregoing pharmaceutical compositions in the manufacture of a medicament. In some embodiments, the use is as described for the methods described herein.

### Chemical Terms

The terminology employed herein is for the purpose of describing particular embodiments and is not intended to be limiting.

For any of the following chemical definitions, a number following an atomic symbol indicates that total number of atoms of that element that are present in a particular chemical moiety. As will be understood, other atoms, such as H atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

The term “acyl,” as used herein, represents a H or an alkyl group that is attached to a parent molecular group through a carbonyl group, as defined herein, and is exemplified by formyl (i.e., a carboxaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms).

An alkylene is a divalent alkyl group. The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6 carbon atoms, or 2 carbon atoms).

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6 carbon atoms, or 2 carbon atoms).

The term “amino,” as used herein, represents —N(R^{N1})₂, wherein each R^{N1} is independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. When polycyclic, the aryl group contains 2 or 3 rings. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Unsubstituted arylalkyl groups contain from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each are further substituted with 1, 2, 3, or 4 substituent groups, valency permitting, as defined herein for the respective groups.

The term “azido,” as used herein, represents a —N₃ group.

The term “bridged polycycloalkyl,” as used herein, refers to a bridged polycyclic group of 5 to 20 carbons, containing from 1 to 3 bridges. A bridged polycycloalkyl group may be unsubstituted or substituted as defined herein for cycloalkyl.

The term “cyano,” as used herein, represents a —CN group.

The term “carbocyclyl,” as used herein, refers to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, and monovalent mono- di-, or tricyclic radical of 3 to 10, preferably 3 to 6 carbon atoms. The cycloalkyl group may be fully saturated or contain 1 or more double or triple bonds, provided that no ring is aromatic. This term is further exemplified by radicals such as cyclopropyl, cyclobutyl, cyclopentyl, cyclohexyl, cycloheptyl, norbornyl, and adamantyl. The term “CH₂-cycloalkyl” as used herein, refers to cycloalkyl- CH₂— groups (e.g cyclopropylmethyl and cyclobutylmethyl).

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group is further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group is further substituted with 1, 2, 3, or 4 substituent groups, valency permitting, as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group is further substituted with 1, 2, 3, or 4 substituent groups, valency permitting, as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to a monocyclic, bicyclic, or tricyclic radical of 5 to 12 atoms having at least one aromatic ring and containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxazolyl, and thiazolyl.

The term “heteroarylalkyl,” as used herein, represents an alkyl group substituted with a heteroaryl group. Unsubstituted heteroarylalkyl groups contain from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each are further substituted with 1, 2, 3, or 4 substituent groups, valency permitting, as defined herein for the respective groups.

The term “heterocyclyl,” as used herein, refers a monocyclic, bicyclic, or tricyclic radical having 3 to 12 atoms having at least one ring containing 1, 2, 3, or 4 ring atoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Unsubstituted heterocyclylalkyl groups contain from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each are further substituted with 1, 2, 3, or 4 substituent groups as defined herein for the respective groups.

The term “hydroxyalkyl,” as used herein, represents an alkyl group substituted with an —OH group.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “oxo,” as used herein, represents a divalent oxygen atom (e.g., the structure of oxo may be shown as ═O). For example, a carbonyl group is a carbon (e.g., alkyl carbon, alkenyl carbon, alkynyl carbon, heteroalkyl carbon, heteroalkenyl carbon, heteroalkynyl carbon, carbocyclyl carbon, etc.) substituted with oxo. Alternatively, sulfur may be substituted with one or two oxo groups (e.g., —SO— or —SO₂— within a substituted heteroalkyl, heteroalkenyl, heteroalkynyl, or heterocyclyl group).

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will be 1, 2, 3, 4, or 5 substituents present, valency permitting, unless otherwise specified. The 1 to 5 substituents are each, independently, selected from the group consisting of acyl, alkyl (e.g., unsubstituted and substituted, where the substituents include any group described herein, e.g., aryl, halo, hydroxy), alkenyl, alkynyl, aryl (e.g., substituted and unsubstituted phenyl), carbocyclyl (e.g., substituted and unsubstituted cycloalkyl), halo (e.g., fluoro), hydroxyl, heteroalkyl (e.g., substituted and unsubstituted methoxy, ethoxy, orthioalkoxy), heteroalkenyl, heteroalkynyl, heteroaryl, heterocyclyl, amino (e.g., NH₂ or mono- or dialkyl amino), azido, cyano, nitro, thiol, and oxo. Each of the substituents is unsubstituted or substituted with unsubstituted substituent(s) as defined herein for each respective group. In some embodiments, alkyl, alkenyl, alkynyl, heteroalkyl, heteroalkenyl, and heteroalkynyl are optionally substituted with 1, 2, 3, 4, or 5 substituents independently selected from the group consisting of aryl (e.g., substituted and unsubstituted phenyl), carbocyclyl (e.g., substituted and unsubstituted cycloalkyl), halo (e.g., fluoro), hydroxyl, heteroaryl, heterocyclyl, amino (e.g., NH₂ or mono- or dialkyl amino), azido, cyano, nitro, thiol, and oxo. Each of the substituents is unsubstituted or substituted with unsubstituted substituent(s) as defined herein for each respective group. In some embodiments, the substituents are themselves unsubstituted.

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent. Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “BAF complex” refers to the BRG1- or HRBM-associated factors complex in a human cell.

As used herein, the term “BAF complex-related disorder” refers to a disorder that is caused or affected by the level of activity of a BAF complex.

As used herein, the term “BRG1 loss of function mutation” refers to a mutation in BRG1 that leads to the protein having diminished activity (e.g., at least 1% reduction in BRG1 activity, for example 2%, 5%, 10%, 25%, 50%, or 100% reduction in BRG1 activity). Exemplary BRG1 loss of function mutations include, but are not limited to, a homozygous BRG1 mutation and a deletion at the C-terminus of BRG1.

As used herein, the term “BRG1 loss of function disorder” refers to a disorder (e.g., cancer) that exhibits a reduction in BRG1 activity (e.g., at least 1% reduction in BRG1 activity, for example 2%, 5%, 10%, 25%, 50%, or 100% reduction in BRG1 activity).

The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

By “determining the level” of a protein or RNA is meant the detection of a protein or an RNA, by methods known in the art, either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure RNA levels are known in the art and include, but are not limited to, quantitative polymerase chain reaction (qPCR) and Northern blot analyses.

By “decreasing the activity of a BAF complex” is meant decreasing the level of an activity related to a BAF complex, or a related downstream effect. A non-limiting example of decreasing an activity of a BAF complex is Sox2 activation. The activity level of a BAF complex may be measured using any method known in the art, e.g., the methods described in Kadoch et al. Cell, 2013, 153, 71-85, the methods of which are herein incorporated by reference.

As used herein, the term “degrader” refers to a small molecule compound including a degradation moiety, wherein the compound interacts with a protein (e.g., BRG1 and/or BRM) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., BRG1 and/or BRM. In one example, the moiety binds to a protease or a ubiquitin ligase that metabolizes the protein, e.g., BRG1 and/or BRM.

By “modulating the activity of a BAF complex,” is meant altering the level of an activity related to a BAF complex (e.g., GBAF), or a related downstream effect. The activity level of a BAF complex may be measured using any method known in the art, e.g., the methods described in Kadoch et al, Cell 153:71-85 (2013), the methods of which are herein incorporated by reference.

By “reducing the activity of BRG1 and/or BRM,” is meant decreasing the level of an activity related to an BRG1 and/or BRM, or a related downstream effect. A non-limiting example of inhibition of an activity of BRG1 and/or BRM is decreasing the level of a BAF complex in a cell. The activity level of BRG1 and/or BRM may be measured using any method known in the art. In some embodiments, an agent which reduces the activity of BRG1 and/or BRM is a small molecule BRG1 and/or BRM degrader.

By “reducing the level of BRG1 and/or BRM,” is meant decreasing the level of BRG1 and/or BRM in a cell or subject. The level of BRG1 and/or BRM may be measured using any method known in the art.

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

As used herein, the term “inhibiting BRM” refers to blocking or reducing the level or activity of the ATPase catalytic binding domain or the bromodomain of the protein. BRM inhibition may be determined using methods known in the art, e.g., a BRM ATPase assay, a Nano DSF assay, or a BRM Luciferase cell assay.

The term “pharmaceutical composition,” as used herein, represents a composition containing a compound described herein formulated with a pharmaceutically acceptable excipient and appropriate for administration to a mammal, for example a human. Typically, a pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration.

As used herein, the term “pharmaceutically acceptable salt” means any pharmaceutically acceptable salt of a compound, for example, any compound of Formula I. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

By a “reference” is meant any useful reference used to compare protein or RNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound of the invention; a sample from a subject that has been treated by a compound of the invention; or a sample of a purified protein or RNA (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound of the invention. In preferred embodiments, the reference sample, standard, or level is matched to the sample subject sample by at least one of the following criteria: age, weight, sex, disease stage, and overall health. A standard curve of levels of a purified protein or RNA, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean therapeutic treatment or any measures whose object is to slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total); an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. Compounds of the invention may also be used to “prophylactically treat” or “prevent” a disorder, for example, in a subject at increased risk of developing the disorder.

The details of one or more embodiments of the invention are set forth in the description below. Other features, objects, and advantages of the invention will be apparent from the description and from the claims.

### DETAILED DESCRIPTION

The present disclosure features compounds useful for the inhibition of BRG1 and optionally BRM. These compounds may be used to modulate the activity of a BAF complex, for example, for the treatment of a BAF-related disorder, such as cancer (e.g., BRG1-loss of function disorders). Exemplary compounds described herein include compounds having a structure according to Formula I, or a pharmaceutically acceptable salt thereof.

The compound of Formula I is:

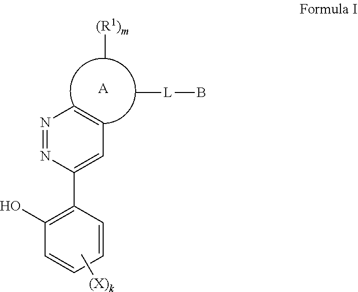

where
ring system A is a 5 to 9-membered heterocyclyl or heteroaryl containing at least one N;
m is 0, 1, 2, or 3;
k is 0, 1, or 2;
each R¹ is, independently, halo, optionally substituted C₁-C₆ alkyl, optionally substituted C₁-C₆ heteroalkyl, optionally substituted C₂-C₉ heterocyclyl, or optionally substituted C₃-C₈ cycloalkyl;
each X is, independently, halo;
L is a linker; and
B is a degradation moiety.

In some embodiments, the compound has the structure of any one of compounds 1-121 in Table 1, or pharmaceutically acceptable salt thereof.

Other embodiments, as well as exemplary methods for the synthesis of production of these compounds, are described herein.

### Pharmaceutical Uses

The compounds described herein are useful in the methods of the invention and, while not bound by theory, are believed to exert their ability to modulate the level, status, and/or activity of a BAF complex, i.e., by inhibiting the activity of the BRG1 and/or BRM proteins within the BAF complex in a mammal. BAF complex-related disorders include, but are not limited to, BRG1 loss of function mutation-related disorders.

An aspect of the present invention relates to methods of treating disorders related to BRG1 loss of function mutations such as cancer (e.g., non-small cell lung cancer, colorectal cancer, bladder cancer, cancer of unknown primary, glioma, breast cancer, melanoma, non-melanoma skin cancer, endometrial cancer, or penile cancer) in a subject in need thereof. In some embodiments, the compound is administered in an amount and for a time effective to result in one or more (e.g., two or more, three or more, four or more) of: (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with a pharmaceutically acceptable salt of the invention.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of the invention. A decrease in the mortality rate of a population may also be measured, for example, by calculating for a population the average number of disease-related deaths per unit time following completion of a first round of treatment with a pharmaceutically acceptable salt of the invention.

Exemplary cancers that may be treated by the invention include, but are not limited to, non-small cell lung cancer, small-cell lung cancer, colorectal cancer, bladder cancer, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer and penile cancer.

### Combination Formulations and Uses Thereof

The compounds of the invention can be combined with one or more therapeutic agents. In particular, the therapeutic agent can be one that treats or prophylactically treats any cancer described herein.

### Combination Therapies

A compound of the invention can be used alone or in combination with an additional therapeutic agent, e.g., other agents that treat cancer or symptoms associated therewith, or in combination with other types of treatment to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, Adriamycin® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), ABraxane®, cremophor-free, albumin-engineered nanoparticle formulation of paclitaxel (American Pharmaceutical Partners, Schaumberg, Ill.), and Taxotere® doxetaxel (Rhone-Poulenc Rorer, Antony, France); chloranbucil; Gemzar® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al. (1999) Proc ASCO 18:233a and Douillard et al. (2000) Lancet 355:1041-7.

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (Avastin®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response or antagonizes an antigen important for cancer. Such agents include Rituxan (Rituximab); Zenapax (Daclizumab); Simulect (Basiliximab); Synagis (Palivizumab); Remicade (Infliximab); Herceptin (Trastuzumab); Mylotarg (Gemtuzumab ozogamicin); Campath (Alemtuzumab); Zevalin (Ibritumomab tiuxetan); Humira (Adalimumab); Xolair (Omalizumab); Bexxar (Tositumomab-I-131); Raptiva (Efalizumab); Erbitux (Cetuximab); Avastin (Bevacizumab); Tysabri (Natalizumab); Actemra (Tocilizumab); Vectibix (Panitumumab); Lucentis (Ranibizumab); Soliris (Eculizumab); Cimzia (Certolizumab pegol); Simponi (Golimumab); Ilaris (Canakinumab); Stelara (Ustekinumab); Arzerra (Ofatumumab); Prolia (Denosumab); Numax (Motavizumab); ABThrax (Raxibacumab); Benlysta (Belimumab); Yervoy (Ipilimumab); Adcetris (Brentuximab Vedotin); Perjeta (Pertuzumab); Kadcyla (Ado-trastuzumab emtansine); and Gazyva (Obinutuzumab). Also included are antibody-drug conjugates.

The second agent may be a therapeutic agent which is a non-drug treatment. For example, the second therapeutic agent is radiation therapy, cryotherapy, hyperthermia and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab). In some embodiments, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MEDI4736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

The compounds of the invention are preferably formulated into pharmaceutical compositions for administration to a mammal, preferably, a human, in a biologically compatible form suitable for administration in vivo. Accordingly, in an aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard- or soft-shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers. A compound of the invention may also be administered parenterally. Solutions of a compound of the invention can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20th ed.) and in The United States Pharmacopeia: The National Formulary (USP 24 NF19), published in 1999. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter. A compound described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

Alternatively, the dosage amount can be calculated using the body weight of the patient. For example, the dose of a compound, or pharmaceutical composition thereof, administered to a patient may range from 0.1-100 mg/kg (e.g., 0.25-25 mg/kg). In exemplary, non-limiting embodiments, the dose may range from 0.5-5.0 mg/kg (e.g., 0.5, 1.0, 1.5, 2.0, 2.5, 3.0, 3.5, 4.0, 4.5, or 5.0 mg/kg) or from 5.0-20 mg/kg (e.g., 5.5, 6.0, 6.5, 7.0, 7.5, 8.0, 8.5, 9.0, 9.5, 10, 11, 12, 13, 14, 15, 16, 17, 18, 19, or 20 mg/kg).

### Examples

The following abbreviations are used throughout the Examples below.
Ac acetyl
ACN or MeCN acetonitrile
AcOH acetic acid
Ac₂O acetic anhydride
aq. aqueous
Boc tert-butoxycarbonyl
Bu or n-Bu butyl
CDI 1,1′-carbonyldiimidazole
DCE or 1,2-DCE 1,2-dichloroethane
DCM dichloromethane
DIAD diisopropyl azodicarboxylate
DIPEA or DIEA N.N-diisopropylethylamine
DMAP 4-(dimethylamino)pyridine
DMB 2,4-dimethoxybenzyl
DME 1,2-dimethoxyethane
DMF N.N-dimethylformamide
DMSO dimethyl sulfoxide
EA or EtOAc ethyl acetate
EDCl N-(3-dimethylaminopropyl)-N′-ethylcarbodiimide hydrochloride
equiv equivalents
Et₃N or TEA triethylamine
EtOH ethyl alcohol
FA formic acid
h or hr hour
HATU 1-[bis(dimethylamino)methylene]-1H-1,2,3-triazolo[4,5-b]pyridinium 3-oxid hexafluorophosphate
HOAt 1-hydroxy-7-azabenzotriazole
HOBt or HOBT 1-hydroxybenzotriazole hydrate
iPr Isopropyl
MeOH methyl alcohol
Me₄t-BuXphos ditert-butyl-[2,3,4,5-tetramethyl-6-(2,4,6-triisopropylphenyl)phenyl]phosphane
min minute
MTBE tert-butyl methyl ether
n-BuLi n-butylithium
NMP 1-methyl-2-pyrrolidinone
OAc acetate
Pd/C palladium on carbon
PDC pyridinium dichromate
PdCl₂(dtbpf) or Pd(dtbpf)Cl₂ dichloro[1,1′-bis(di-t-butylphosphino)ferrocene]palladium(II)
PdCl₂(dppf) or Pd(dppf)Cl_{2 [1,1}′-bis(diphenylphosphino)ferrocene]dichloropalladium(II)
Pd₂(dba)₃ tris(dibenzylideneacetone)dipalladium(0)
Pd(PPh₃)₄ tetrakis(triphenylphosphine)palladium(0
Pd(PPh₃)₂Cl₂ dichlorobis(triphenylphosphine)palladium(II)
PE petroleum ether
PPh₃ triphenylphosphine
Pr n-propyl
Py pyridine
rac racemic
Rf retention factor
r.t. or rt room temperature
sat. saturated
SFC supercritical fluid chromatography
t-Bu tert-butyl
tBuXphos-Pd-G3 or tBuXphos Pd G₃ or t-BuXphos-Pd (gen 3) [2-(2-aminophenyl)phenyl]-methylsulfonyloxypalladium;ditert-butyl-[2-(2,4,6-triisopropylphenyl)phenyl]phosphane
TFA trifluoroacetic acid
Tf₂O trifluoromethanesulfonic anhydride
THF tetrahydrofuran
TLC thin layer chromatography
Xantphos-Pd-G3 [2-(2-aminophenyl)phenyl]-methylsulfonyloxy-palladium;(5-diphenylphosphanyl-9,9-dimethyl-xanthen-4-yl)-diphenyl-phosphane
XPhos Pd G3 (2-Dicyclohexylphosphino-2′,4′,6′-triisopropyl-1,1′-biphenyl)[2-(2′-amino-1,1′-biphenyl)]palladium(II) methanesulfonate

### Example 1. Preparation of Compounds

### Preparation of (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (I-1)

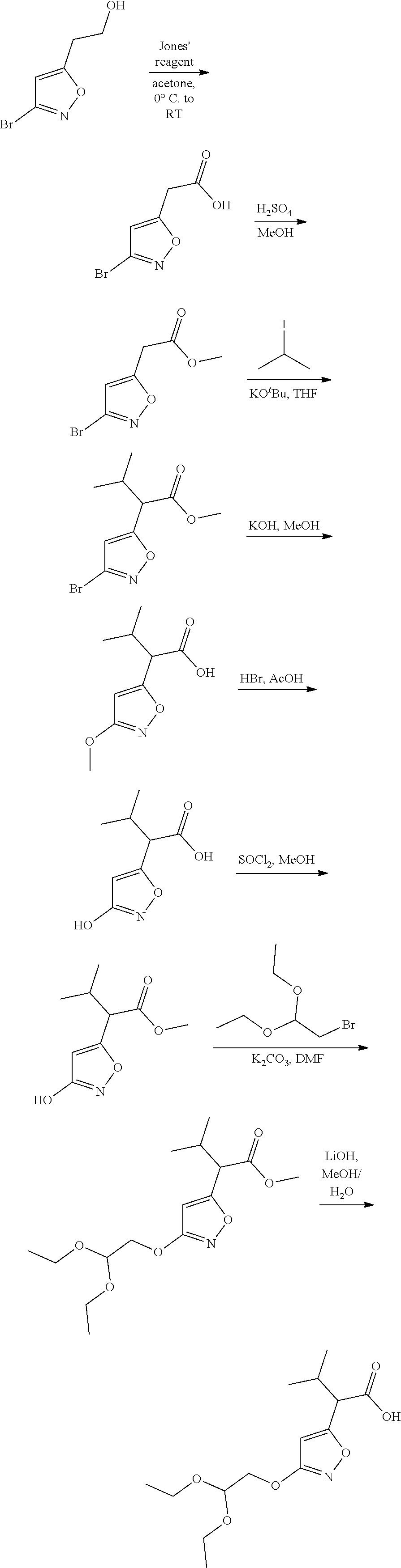

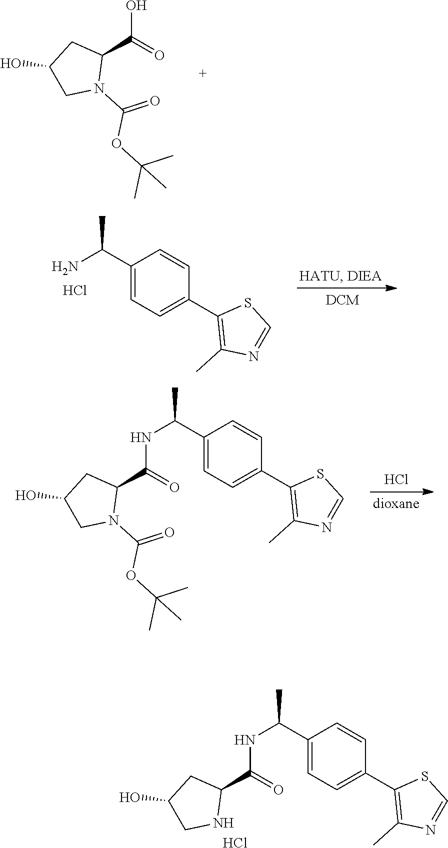

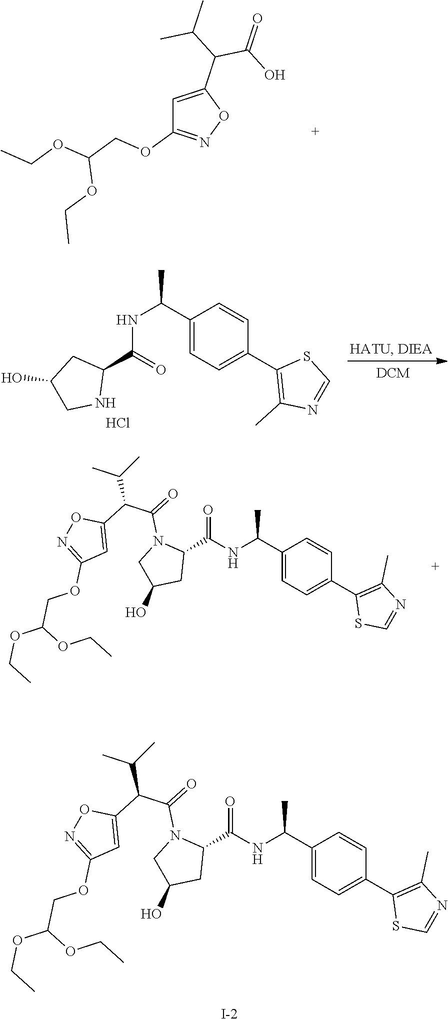

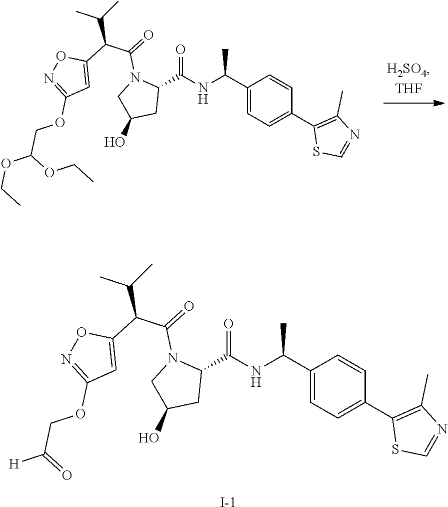 [SMILES: CCOC(COc1cc([C@H](C(=O)N2C[C@H](O)C[C@H]2C(=O)N[C@@H](C)c2ccc(-c3scnc3C)cc2)C(C)C)on1)OCC ; Cc1ncsc1-c1ccc([C@H](C)NC(=O)[C@@H]2C[C@@H](O)CN2C(=O)[C@@H](c2cc(OCC=O)no2)C(C)C)cc1 ; [I-]]

### Step 1: Preparation of 2-(3-bromoisoxazol-5-yl)acetic acid

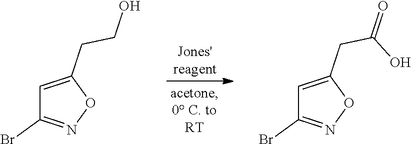 [SMILES: OCCc1cc(Br)no1 ; O=C(O)Cc1cc(Br)no1]

To a stirring solution of 2-(3-bromo-1,2-oxazol-5-yl)ethan-1-ol (30 g, 156 mmol) in acetone (389 mL) was added Jones' reagent (2 M in acetone, 156 mL, 312 mmol) dropwise at 0° C. The resulting solution was stirred at 25° C. overnight. The mixture was diluted with water and extracted with EtOAc. The organic layer was washed with brine, and dried over anhydrous Na₂SO₄ and concentrated under reduced pressure to afford 2-(3-bromoisoxazol-5-yl)acetic acid as a brown solid (28 g, 86.5%). LCMS (ESI) m/z: [M+H]⁺=206.08 and 208.08.

### Step 2: Preparation of methyl 2-(3-bromoisoxazol-5-yl)acetate

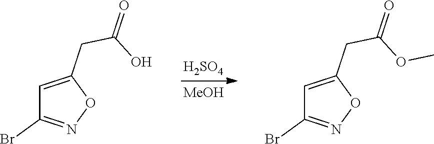

A solution of 2-(3-bromoisoxazol-5-yl)acetic acid (28 g, 135 mmol) and concentrated H₂SO₄ (3 mL, 72 mmol) in methanol (250 mL) was stirred at 70° C. for 2 h. The resulting solution was concentrated under reduced pressure. The residue was diluted with water and extracted with EtOAc. The organic layer was washed with brine, and dried over anhydrous MgSO₄ and concentrated under reduced pressure. The residue was purified by silica gel flash chromatography (EtOAc/petroleum ether) to afford methyl 2-(3-bromoisoxazol-5-yl)acetate as a white solid (23.4 g, 79%). LCMS (ESI) m/z: [M+H]⁺=219.90 and 221.86.

### Step 3: Preparation of methyl 2-(3-bromoisoxazol-5-yl)-3-methylbutanoate

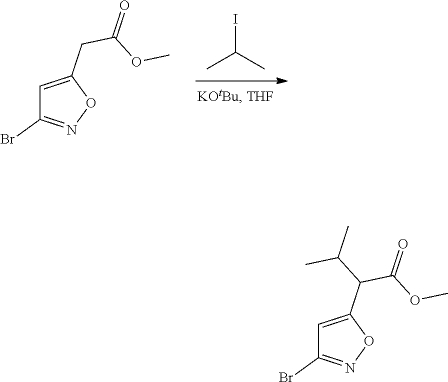

To a stirred solution of methyl 2-(3-bromoisoxazol-5-yl)acetate (23.4 g, 106 mmol) and KOtBu (17.8 g, 159 mmol) in THF (210 mL) was added 2-iodopropane (13.8 mL, 137 mmol) dropwise at 0° C. The reaction mixture was stirred at room temperature for 16 h and then quenched with water/ice. The resulting solution was extracted several times with EtOAc. The combined organic layers were washed with brine, and dried over anhydrous Na₂SO₄ and concentrated under reduced pressure. The residue was purified by silica gel flash chromatography (EtOAc/petroleum ether) to afford methyl 2-(3-bromoisoxazol-5-yl)-3-methylbutanoate as a clear oil (16.7 g, 60%).

### Step 4: Preparation of 2-(3-methoxyisoxazol-5-yl)-3-methylbutanoic acid

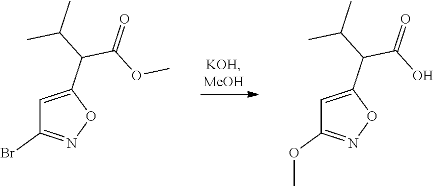

To a solution of methyl 2-(3-bromo-1,2-oxazol-5-yl)-3-methylbutanoate (16.7 g, 63.7 mmol) in methanol (130 mL) was added potassium hydroxide (35.7 g, 637 mmol). The mixture was stirred for 4 h at 100° C. The mixture was concentrated under vacuum and then diluted with water. The resulting solution was washed with EtOAc and pH of the aqueous layer was adjusted to pH 5 with 1 N HCl. This mixture was extracted several times with EtOAc. The combined organic layers were washed with brine and dried over anhydrous MgSO₄. The residue was purified by silica gel flash chromatography (EtOAc/petroleum ether) to afford 2-(3-methoxyisoxazol-5-yl)-3-methylbutanoic acid as a yellow oil (8.8 g, 70%). LCMS (ESI) m/z: [M+H]⁺=200.15.

### Step 5: Preparation of 2-(3-hydroxyisoxazol-5-yl)-3-methylbutanoic acid

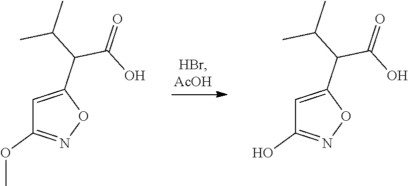

A solution of 2-(3-methoxyisoxazol-5-yl)-3-methylbutanoic acid (8.8 g, 44.1 mmol) in HOAc (80 mL) and HBr (80 mL) was stirred at 60° C. for 16 h. The resulting mixture was concentrated under reduced pressure to afford crude 2-(3-hydroxyisoxazol-5-yl)-3-methylbutanoic acid (8.16 g, quant.).

### Step 6: Preparation of methyl 2-(3-hydroxyisoxazol-5-yl)-3-methylbutanoate

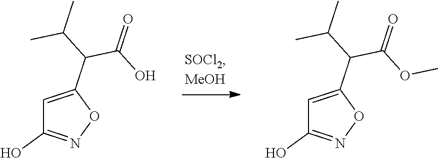

To a solution of 2-(3-hydroxy-1,2-oxazol-5-yl)-3-methylbutanoic acid (8.16 g, 44.0 mmol) in methanol (30 mL) was slowly added SOCl₂ (14.2 mL, 197 mmol). The mixture was stirred at room temperature for 3 h. The solvent was removed under reduced pressure. The residue was diluted with water and extracted with EtOAc. The organic layer was washed with brine, dried over anhydrous Na₂SO₄ and concentrated under reduced pressure. The residue was purified by silica gel flash chromatography (MeOH/DCM) to afford methyl 2-(3-hydroxyisoxazol-5-yl)-3-methylbutanoate as a clear oil (7.79 g, 89%). LCMS (ESI) m/z: [M+H]⁺=200.15.

### Step 7: Preparation of methyl 2-(3-(2,2-diethoxyethoxy)isoxazol-5-yl)-3-methylbutanoate

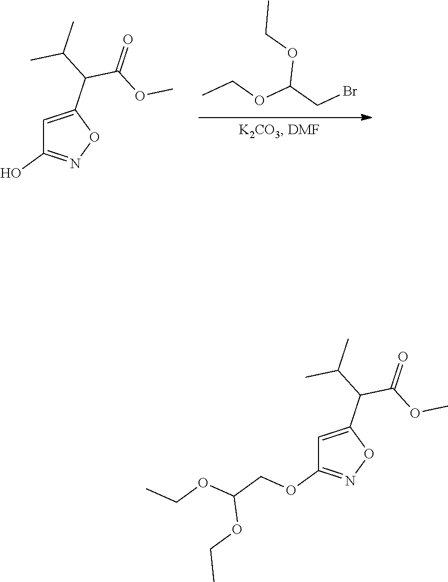

To a solution of methyl 2-(3-hydroxy-1,2-oxazol-5-yl)-3-methylbutanoate (7.79 g, 39.1 mmol) in DMF (90 mL) were added 2-bromo-1,1-diethoxyethane (8.77 mL, 58.6 mmol) and potassium carbonate (10.8 g, 78.2 mmol). The reaction was stirred at 70° C. overnight. The reaction mixture was cooled and then water was added to the mixture. The resulting mixture was extracted with EtOAc several times. The combined organic layers were washed with brine and dried over anhydrous MgSO₄. Solvent was removed under reduced pressure and the resulting residue was purified by silica gel flash chromatography (EtOAc/heptane) to afford methyl 2-(3-(2,2-diethoxyethoxy)isoxazol-5-yl)-3-methylbutanoate as a colorless oil (7.8 g, 63%). LCMS (ESI) m/z: [M-C₂H₅O]⁺=270.30.

### Step 8: Preparation of 2-(3-(2,2-diethoxyethoxy)isoxazol-5-yl)-3-methylbutanoic acid

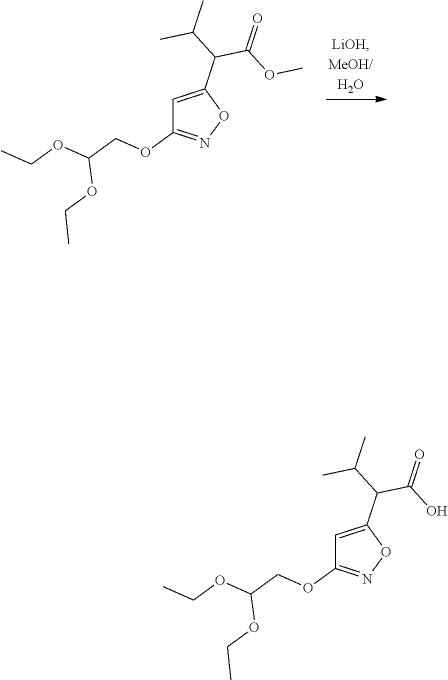

To a solution of methyl 2-[3-(2,2-diethoxyethoxy)-1,2-oxazol-5-yl]-3-methylbutanoate (7.8 g, 24.7 mmol) in methanol (50 mL) and water (25 mL) was added lithium hydroxide monohydrate (4.14 g, 98.8 mmol). The reaction was stirred at 40° C. for 2 h. The pH was adjusted to 4-5 with 1 N HCl. The mixture was extracted with ethyl acetate several times and combined organic layers were dried over MgSO₄. Solvents were removed under reduced pressure and the residue was purified by silica gel flash chromatography (DCM/MeOH) to afford 2-(3-(2,2-diethoxyethoxy)isoxazol-5-yl)-3-methylbutanoic acid as a colorless oil (6.1 g, 89%). LCMS (ESI) m/z: [M−H]⁻=300.21.

### Step 9: Preparation of tert-butyl (2S,4R)-4-hydroxy-2-(((S)-1-(4-(4-methylthiazol-5-yl)phenyl)ethyl)carbamoyl)pyrrolidine-1-carboxylate

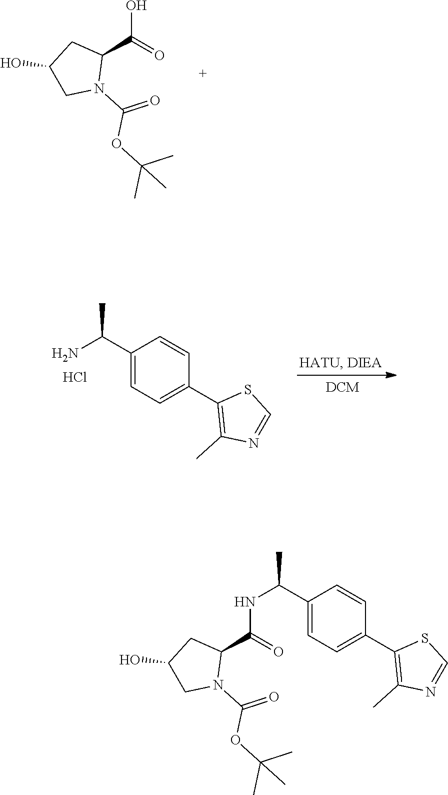 [SMILES: CC(C)(C)OC(=O)N1C[C@H](O)C[C@H]1C(=O)O ; Cl ; Cc1ncsc1-c1ccc([C@H](C)N)cc1 ; Cc1ncsc1-c1ccc([C@H](C)NC(=O)[C@@H]2C[C@@H](O)CN2C(=O)OC(C)(C)C)cc1]

To a solution of (S)-1-(4-(4-methylthiazol-5-yl)phenyl)ethan-1-amine hydrochloride (5.0 g, 19.6 mmol) and (2S,4R)-1-[(tert-butoxy)carbonyl]-4-hydroxypyrrolidine-2-carboxylic acid (4.47 g, 20.5 mmol) in DCM (70 mL) at 0° C. was added HATU (8.98 g, 23.5 mmol) followed by dropwise addition of DIEA (16.4 mL, 98.0 mmol). After stirring for 16 h at room temperature, the reaction mixture was poured into ice water. The resulting mixture was extracted several times with DCM. The combined organic layers were washed with water, brine, and dried over anhydrous Na₂SO₄ and concentrated under vacuum. The resulting residue was purified by silica gel flash chromatography (MeOH/DCM) to afford tert-butyl (2S,4R)-4-hydroxy-2-(((S)-1-(4-(4-methylthiazol-5-yl)phenyl)ethyl)carbamoyl)pyrrolidine-1-carboxylate (8.33 g, 98%). LCMS (ESI) m/z: [M+H]⁺=432.38.

### Step 10: Preparation of (2S,4R)-4-hydroxy-N—((S)-1-(4-(4-methylthiazol-5-yl)phenyl)ethyl)pyrrolidine-2-carboxamide hydrochloride

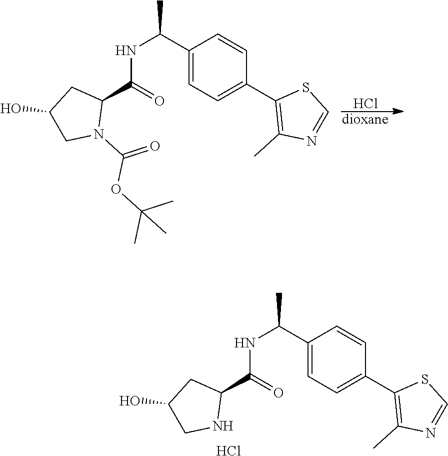 [SMILES: Cc1ncsc1-c1ccc([C@H](C)NC(=O)[C@@H]2C[C@@H](O)CN2C(=O)OC(C)(C)C)cc1 ; Cl ; Cc1ncsc1-c1ccc([C@H](C)NC(=O)[C@@H]2C[C@@H](O)CN2)cc1 ; Cl]

To tert-butyl (2S,4R)-4-hydroxy-2-(((S)-1-(4-(4-methylthiazol-5-yl)phenyl)ethyl)carbamoyl)pyrrolidine-1-carboxylate (8.33 g, 19.3 mmol) at 0° C. was added a solution of HCl in 1,4-dioxane (4 N, 50 mL, 200 mmol) resulting in a sticky yellow gum. 15 mL of MeOH were added to the mixture and the mixture was stirred at room temperature for 2 h. The solvents were removed under reduced pressure and the residue was washed with diethyl ether to afford (2S,4R)-4-hydroxy-N—((S)-1-(4-(4-methylthiazol-5-yl)phenyl)ethyl)pyrrolidine-2-carboxamide hydrochloride which was used in the next step without further purification.

### Step 11: Preparation of (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide (I-2)

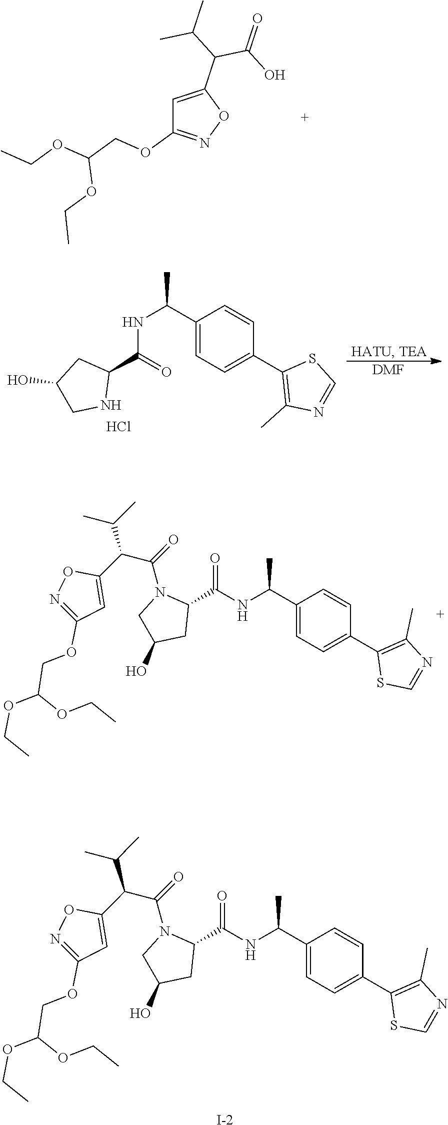

To a solution of 2-[3-(2,2-diethoxyethoxy)isoxazol-5-yl]-3-methyl-butanoic acid (5.75 g, 19.0 mmol) in DMF (30 mL) was added HATU (8.6 g, 22.7 mmol). After stirring at 20° C. for 0.5 h, a solution of (2S,4R)-4-hydroxy-N-[(1S)-1-[4-(4-methylthiazol-5-yl)phenyl]ethyl]pyrrolidine-2-carboxamide hydrochloride (6.97 g, 19.0 mmol) and triethylamine (7.92 mL, 56.9 mmol) in DMF (20 mL) was added to the mixture and the resulting mixture was stirred at 20° C. The reaction mixture was quenched by addition of water and extracted several times with EtOAc. The combined organic layers were washed with brine, and dried over anhydrous MgSO₄, filtered and concentrated under reduced pressure. The residue was purified by silica gel flash chromatography (DCM/MeOH) to afford (2S,4R)-1-[2-[3-(2,2-diethoxyethoxy)isoxazol-5-yl]-3-methylbutanoyl]-4-hydroxy-N-[(1S)-1-[4-(4-methylthiazol-5-yl)phenyl]ethyl]pyrrolidine-2-carboxamide (10 g, 16.2 mmol) as a white solid. The mixture of diastereomers was separated by chiral SFC chromatography to afford (2S,4R)-1-((S)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide and (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide.

(2S,4R)-1-((S)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide peak 1: (2.2 g, 19%). LCMS (ESI) m/z [M+H]⁺=615.4.

(2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide (I-2) peak 2: (2.5 g, 21%). LCMS (ESI) m/z [M+H]⁺=615.4.

### Step 12: Preparation of (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (I-1)

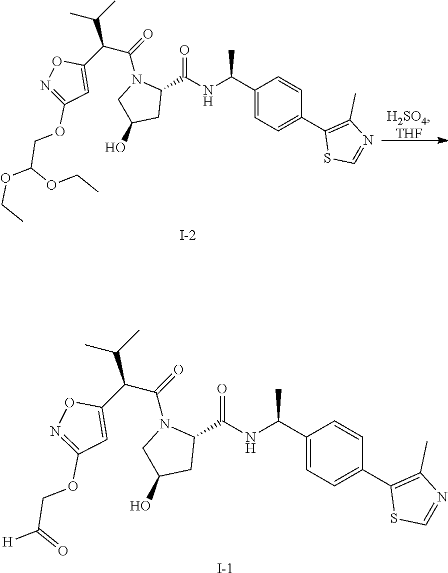

To a stirred solution of H₂SO₄ (1 N, 6.00 mL) and THF (6.00 mL) was added (2S,4R)-1-[(2R)-2-[3-(2-ethoxy-2-methoxyethoxy)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (I-2, 300 mg, 0.499 mmol) in portions at room temperature. The resulting mixture was stirred for 8 h at 50° C. The resulting mixture was diluted with water, then neutralized to pH ˜7 with saturated aqueous NaHCO₃. The resulting mixture was extracted three times with EtOAc. The combined organic layers were washed twice with brine and dried over anhydrous Na₂SO₄. After filtration, the filtrate was concentrated under reduced pressure to afford (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (I-1, 256 mg, 97.3%) as a white solid. LCMS (ESI) m/z: [M+H]⁺=541.

The following intermediate in Table 2 was prepared in a similar manner as described in the preparation of intermediate I-1 starting with methyl 2-(3-hydroxy-1,2-oxazol-5-yl)-3-methylbutanoate and the appropriate alkyl bromide.

TABLE 2

| | Inter- | | LCMS |
|---|---|---|---|
| | mediate | | (ESI) |
| Structure | No. | Name | m/z |
| 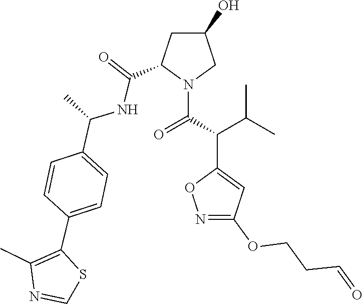 [SMILES: Cc1ncsc1-c1ccc([C@H](C)NC(=O)[C@@H]2C[C@@H](O)CN2C(=O)[C@@H](c2cc(OCCC=O)no2)C(C)C)cc1] | I-3 | (2S,4R)-4-hydroxy-1-((R)-3- methyl-2-(3-(3- oxopropoxy)isoxazol-5- yl)butanoyl)-N-((S)-1-(4-(4- methylthiazol-5- yl)phenyl)ethyl)pyrrolidine-2- carboxamide | 555 |

### Preparation of (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (I-5) and (2S,4R)-4-hydroxy-N-[(1S)-1-[4-(4-methyl-1,3-thiazol-5-yl)phenyl]ethyl]-1-[(2S)-3-methyl-2-[3-(piperazin-1-yl)-1,2-oxazol-5-yl]butanoyl]pyrrolidine-2-carboxamide (I-4)

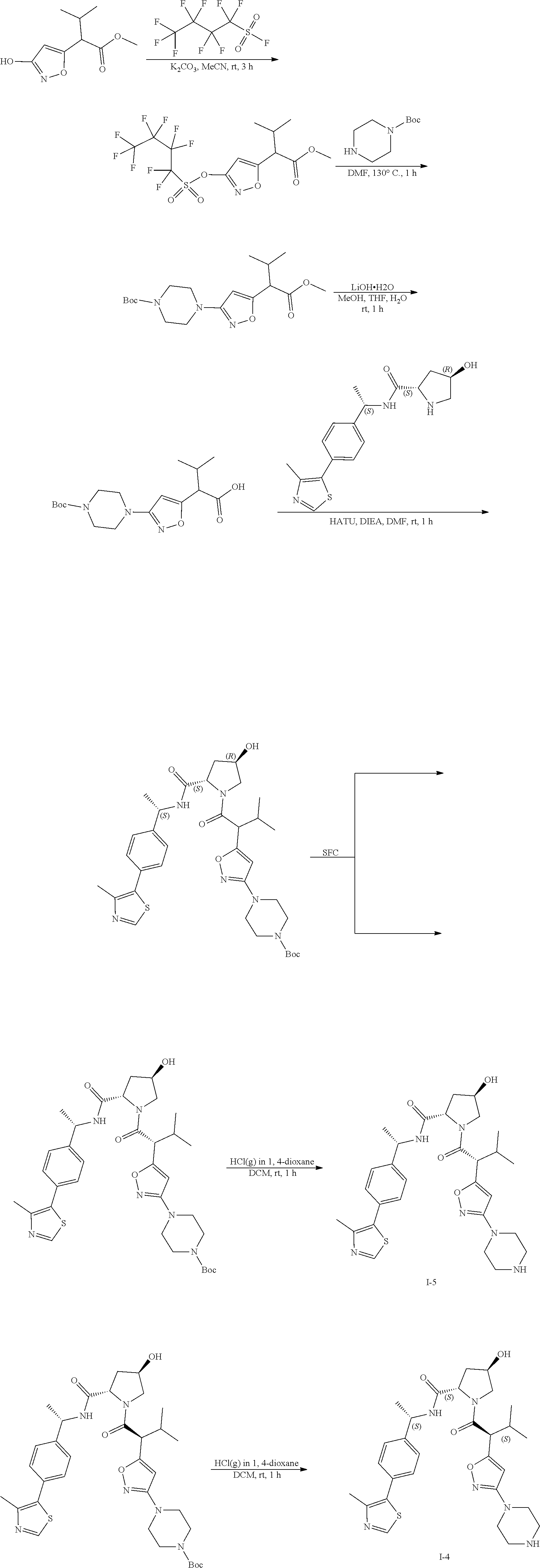

### Step 1: Preparation of methyl 3-methyl-2-[3-[(1,1,2,2,3,3,4,4,4-nonafluorobutanesulfonyl)oxy]-1,2-oxazol-5-yl]butanoate

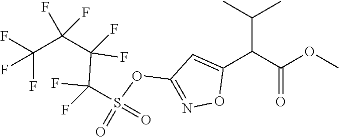 [SMILES: COC(=O)C(c1cc(OS(=O)(=O)C(F)(F)C(F)(F)C(F)(F)C(F)(F)F)no1)C(C)C]

To a stirred solution of methyl 2-(3-hydroxy-1,2-oxazol-5-yl)-3-methylbutanoate (100.00 mg, 0.502 mmol, 1.00 equiv) in MeCN (0.50 mL) were added perfluorobutanesulfonyl fluoride (303.29 mg, 1.004 mmol, 2.00 equiv) and K₂CO₃ (208.13 mg, 1.506 mmol, 3.00 equiv) at room temperature. The resulting mixture was stirred for 3 h, then carefully quenched with water at 0 degrees C. The resulting mixture was extracted with EA (2×50 mL). The combined organic layers were washed with brine (50 mL) and dried over anhydrous Na₂SO₄. After filtration, the filtrate was concentrated under reduced pressure. The residue was purified by silica gel column chromatography, eluting with PE/EA (2/1) to afford methyl 3-methyl-2-[3-[(1,1,2,2,3,3,4,4,4-nonafluorobutanesulfonyl)oxy]-1,2-oxazol-5-yl]butanoate (217 mg) as a white solid. LCMS (ESI) m/z: [M+H]⁺=482.

### Step 2: Preparation of tert-butyl 4-[5-(1-methoxy-3-methyl-1-oxobutan-2-yl)-1,2-oxazol-3-yl]piperazine-1-carboxylate

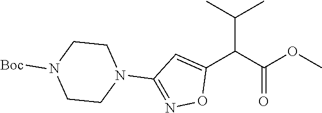 [SMILES: COC(=O)C(c1cc(N2CCN(C(=O)OC(C)(C)C)CC2)no1)C(C)C]

To a stirred solution of methyl 3-methyl-2-[3-[(1,1,2,2,3,3,4,4,4-nonafluorobutanesulfonyl)oxy]-1,2-oxazol-5-yl]butanoate (217.00 mg, 0.451 mmol, 1.00 equiv) in DMF (3.00 mL) was added tert-butyl piperazine-1-carboxylate (83.98 mg, 0.451 mmol, 1.00 equiv) at room temperature. The resulting mixture was stirred for 1 h at 130° C. The mixture was allowed to cool down to room temperature. The residue was purified by reverse phase flash chromatography with the following conditions: column, C18 silica gel; mobile phase, MeCN in water (0.1% FA), 0 to 100% gradient in 30 min. This provided tert-butyl 4-[5-(1-methoxy-3-methyl-1-oxobutan-2-yl)-1,2-oxazol-3-yl]piperazine-1-carboxylate (54 mg, 32.59%) as a yellow oil. LCMS (ESI) m/z: [M+H]⁺=368.

### Step 3: Preparation of 2-[3-[4-(tert-butoxycarbonyl)piperazin-1-yl]-1,2-oxazol-5-yl]-3-methylbutanoic acid

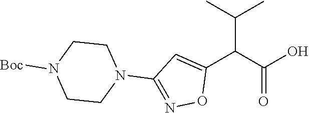 [SMILES: CC(C)C(C(=O)O)c1cc(N2CCN(C(=O)OC(C)(C)C)CC2)no1]

To a stirred solution of tert-butyl 4-[5-(1-methoxy-3-methyl-1-oxobutan-2-yl)-1,2-oxazol-3-yl]piperazine-1-carboxylate (54.00 mg, 0.147 mmol, 1.00 equiv) in MeOH (0.80 mL) were added THF (0.80 mL) and H₂O (0.80 mL) at room temperature, followed by addition of LiOH·H₂O (18.50 mg, 0.441 mmol, 3.00 equiv). The resulting mixture was stirred for 1 h at room temperature. The mixture was acidified to pH 6 with HCl (1 M, aq.), then extracted with EA (2×50 mL). The combined organic layers were washed with brine (50 mL), and dried over anhydrous Na₂SO₄, and filtered. The filtrate was concentrated under reduced pressure. This provided 2-[3-[4-(tert-butoxycarbonyl)piperazin-1-yl]-1,2-oxazol-5-yl]-3-methylbutanoic acid (52 mg, crude product) as a yellow solid. LCMS (ESI) m/z: [M+H]⁺=354.

### Step 4: Preparation of tert-butyl 4-(5-[1-[(2S,4R)-4-hydroxy-2-[[(1S)-1-[4-(4-methyl-1,3-thiazol-5-yl)phenyl]ethyl]carbamoyl]pyrrolidin-1-yl]-3-methyl-1-oxobutan-2-yl]-1,2-oxazol-3-yl)piperazine-1-carboxylate

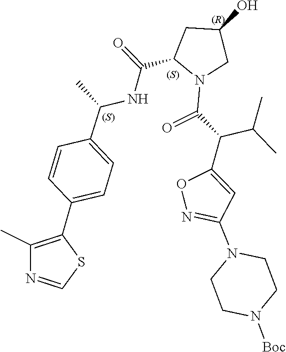

To a stirred solution of 2-[3-[4-(tert-butoxycarbonyl)piperazin-1-yl]-1,2-oxazol-5-yl]-3-methylbutanoic acid (52.00 mg, 0.119 mmol, 1.00 equiv) in DMF (2.00 mL) were added HATU (135.56 mg, 0.357 mmol, 3.00 equiv) and DIEA (76.80 mg, 0.595 mmol, 5.00 equiv) at room temperature. To the above mixture was added (2S,4R)-4-hydroxy-N-[(1S)-1-[4-(4-methyl-1,3-thiazol-5-yl)phenyl]ethyl]pyrrolidine-2-carboxamide (70.90 mg, 0.214 mmol, 1.80 equiv) at room temperature. The resulting mixture was stirred for 1 h. The mixture was purified directly by reverse phase flash chromatography with the following conditions: column, C18 silica gel; mobile phase, MeCN in water (0.1% FA), 0 to 100% gradient in 30 min. This provided tert-butyl 4-(5-[1-[(2S,4R)-4-hydroxy-2-[[(1S)-1-[4-(4-methyl-1,3-thiazol-5-yl)phenyl]ethyl]carbamoyl]pyrrolidin-1-yl]-3-methyl-1-oxobutan-2-yl]-1,2-oxazol-3-yl)piperazine-1-carboxylate (73 mg, 92.12%) as a white solid. LCMS (ESI) m/z: [M+H]⁺=667.

### Step 5: Preparation of tert-butyl 4-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carboxylate and tert-butyl 4-(5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carboxylate

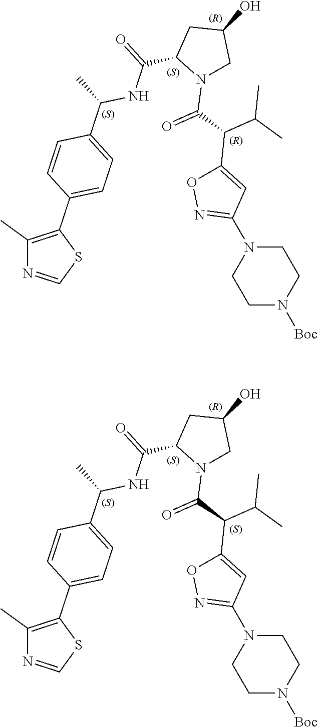

tert-butyl 4-(5-[1-[(2S,4R)-4-hydroxy-2-[[(1S)-1-[4-(4-methyl-1,3-thiazol-5-yl)phenyl]ethyl]carbamoyl]pyrrolidin-1-yl]-3-methyl-1-oxobutan-2-yl]-1,2-oxazol-3-yl)piperazine-1-carboxylate (73 mg) was purified by SFC with the following conditions: Column, CHIRAL ART Amylose-C NEO, 3*25 cm, 5 μm; mobile phase, MeOH.

This provided:

tert-butyl 4-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carboxylate (37 mg, second peak). LCMS (ESI) m/z: [M+H]⁺=667.

tert-butyl 4-(5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carboxylate (34 mg, first peak). LCMS (ESI) m/z: [M+H]⁺=667.

### Step 6: Preparation of (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (I-5) and (2S,4R)-4-hydroxy-N-[(1S)-1-[4-(4-methyl-1,3-thiazol-5-yl)phenyl]ethyl]-1-[(2S)-3-methyl-2-[3-(piperazin-1-yl)-1,2-oxazol-5-yl]butanoyl]pyrrolidine-2-carboxamide (I-4)

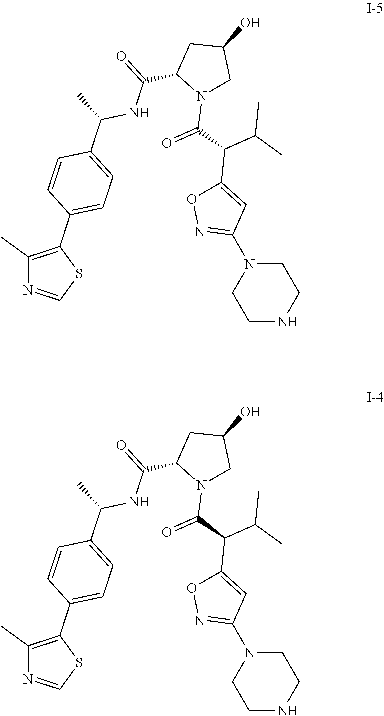 [SMILES: Cc1ncsc1-c1ccc([C@H](C)NC(=O)[C@@H]2C[C@@H](O)CN2C(=O)[C@@H](c2cc(N3CCNCC3)no2)C(C)C)cc1 ; Cc1ncsc1-c1ccc([C@H](C)NC(=O)[C@@H]2C[C@@H](O)CN2C(=O)[C@H](c2cc(N3CCNCC3)no2)C(C)C)cc1]

To a stirred solution of tert-butyl 4-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carboxylate (37.00 mg, 0.055 mmol, 1.00 equiv) in DCM (1.50 mL) was added HCl in 1,4-dioxane (1.50 mL, 26.276 mmol, 473.57 equiv) at 0° C. The resulting mixture was stirred for 1 h at room temperature, then was concentrated under reduced pressure. This provided 1-5 (45 mg, crude product) as a yellow oil. LCMS (ESI) m/z: [M+H]⁺=567.

I-4 was prepared following the same protocol as 1-5 and was obtained as a yellow oil. LCMS (ESI) m/z: [M+H]⁺=567.

The following intermediates in Table 3 were prepared in a similar manner as described in the preparation of intermediate I-5 starting with methyl 3-methyl-2-[3-[(1,1,2,2,3,3,4,4,4-nonafluorobutanesulfonyl)oxy]-1,2-oxazol-5-yl]butanoate and the appropriate amines.

TABLE 3

| | Inter- | | LCMS |
|---|---|---|---|
| | mediate | | (ESI) |
| Structure | No. | Name | m/z |
| 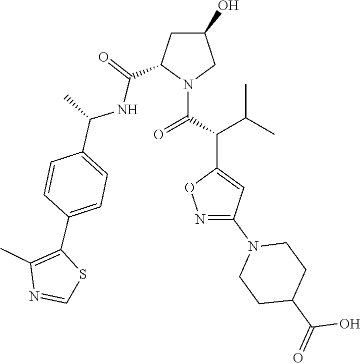 [SMILES: Cc1ncsc1-c1ccc([C@H](C)NC(=O)[C@@H]2C[C@@H](O)CN2C(=O)[C@@H](c2cc(N3CCC(C(=O)O)CC3)no2)C(C)C)cc1] | I-6 | 1-{5-[(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3-methyl-1- oxobutan-2-yl]-1,2-oxazol-3- yl}piperidine-4-carboxylic acid | 610 |
| 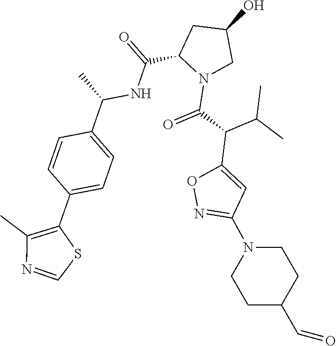 [SMILES: Cc1ncsc1-c1ccc([C@H](C)NC(=O)[C@@H]2C[C@@H](O)CN2C(=O)[C@@H](c2cc(N3CCC(C=O)CC3)no2)C(C)C)cc1] | I-7 | (2S,4R)-1-[(2R)-2-[3-(4- formylpiperidin-1-yl)-1,2- oxazol-5-yl]-3-methylbutanoyl]- 4-hydroxy-N-[(1S)-1-[4-(4- methyl-1,3-thiazol-5- yl)phenyl]ethyl]pyrrolidine-2- carboxamide | 594 |
| 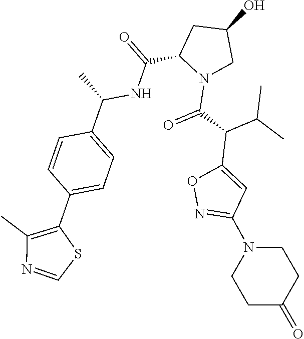 [SMILES: Cc1ncsc1-c1ccc([C@H](C)NC(=O)[C@@H]2C[C@@H](O)CN2C(=O)[C@@H](c2cc(N3CCC(=O)CC3)no2)C(C)C)cc1] | I-8 | (2S,4R)-4-hydroxy-1-((R)-3- methyl-2-(3-(4-oxopiperidin-1- yl)isoxazol-5-yl)butanoyl)-N- ((S)-1-(4-(4-methylthiazol-5- yl)phenyl)ethyl)pyrrolidine-2- carboxamide | 580 |
| 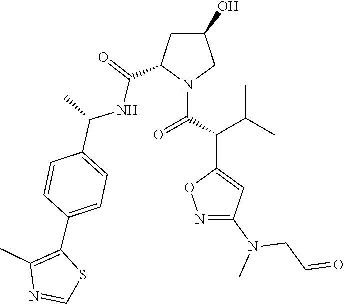 [SMILES: Cc1ncsc1-c1ccc([C@H](C)NC(=O)[C@@H]2C[C@@H](O)CN2C(=O)[C@@H](c2cc(N(C)CC=O)no2)C(C)C)cc1] | I-9 | (2S,4R)-4-hydroxy-1-((R)-3- methyl-2-(3-(methyl(2- oxoethyl)amino)isoxazol-5- yl)butanoyl)-N-((S)-1-(4-(4- methylthiazol-5- yl)phenyl)ethyl)pyrrolidine-2- carboxamide | 554 |

### Preparation of methyl 2-[3-(2-chloropyrimidin-5-yl)-1,2-oxazol-5-yl]-3-methylbutanoate

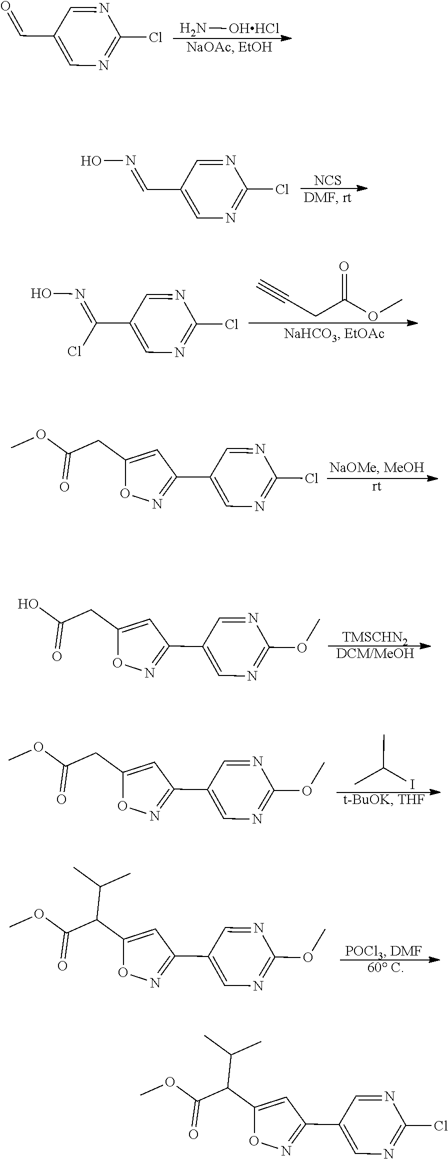

### Step 1: Preparation of (E)-N-[(2-chloropyrimidin-5-yl)methyl idene]hydroxylamine

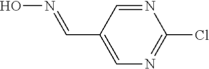

To a stirred solution of 2-chloropyrimidine-5-carbaldehyde (5 g, 35.078 mmol, 1 equiv) and NH₂OH HCl (4.93 g, 70.945 mmol, 2.02 equiv) in EtOH (250 mL) was added NaOAc (14.48 g, 176.512 mmol, 5.03 equiv) at room temperature. The resulting mixture was stirred for 2 h at room temperature. The solvent was removed under reduced pressure. The residue was dissolved in EtOAc (500 mL), washed with brine (500 mL), and dried over anhydrous Na₂SO₄. After filtration, the filtrate was concentrated under reduced pressure to afford (E)-N-[(2-chloropyrimidin-5-yl)methylidene]hydroxylamine (4.6 g, crude product) as a light yellow solid which was used in the next step directly without further purification. LCMS (ESI) m/z: [M+H]⁺=158.

### Step 2: Preparation of (Z)-2-chloro-N-hydroxypyrimidine-5-carbonimidoyl chloride

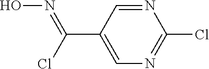 [SMILES: O/N=C(\Cl)c1cnc(Cl)nc1]

A solution of (E)-N-[(2-chloropyrimidin-5-yl)methylidene]hydroxylamine (4.6 g, 29.195 mmol, 1 equiv) and NCS (4.4 g, 32.951 mmol, 1.13 equiv) in DMF (150 mL) was stirred for 2 h at room temperature. The mixture was diluted with EtOAc (500 mL). The resulting mixture was washed with water (3×300 mL), brine (1×300 mL) and the organic phase was dried over anhydrous Na₂SO₄. After filtration, the filtrate was concentrated under reduced pressure to afford (Z)-2-chloro-N-hydroxypyrimidine-5-carbonimidoyl chloride (4.8 g, crude product) as a yellow solid. LCMS (ESI) m/z: [M+H]⁺=192.

### Step 3: Preparation of methyl 2-[3-(2-chloropyrimidin-5-yl)-1,2-oxazol-5-yl]acetate

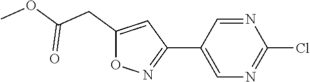 [SMILES: COC(=O)Cc1cc(-c2cnc(Cl)nc2)no1]

A solution of (Z)-2-chloro-N-hydroxypyrimidine-5-carbonimidoyl chloride (4.8 g, 25.00 mmol, 1 equiv) in EtOAc (80 mL) was treated with NaHCO₃ (3 g, 35.712 mmol, 1.43 equiv) for 30 min at 0° C. under an atmosphere of dry nitrogen followed by the addition of methyl but-3-ynoate (2.02 g, 20.591 mmol, 0.82 equiv) in portions at 0° C. The resulting mixture was stirred for 12 h at room temperature. The resulting mixture was diluted with water (150 mL) and extracted with EtOAc (2×400 mL). The combined organic layers were washed with brine (1×400 mL) and dried over anhydrous Na₂SO₄. After filtration, the filtrate was concentrated under reduced pressure. The residue was purified by silica gel column chromatography, eluted with PE/EA (3:1) to afford methyl 2-[3-(2-chloropyrimidin-5-yl)-1,2-oxazol-5-yl]acetate (2.5 g, 38.64%) as a light yellow solid. LCMS (ESI) m/z: [M+H]⁺=254.

### Step 4: Preparation of [3-(2-methoxypyrimidin-5-yl)-1,2-oxazol-5-yl]acetic acid

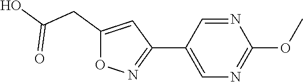 [SMILES: COc1ncc(-c2cc(CC(=O)O)on2)cn1]

A solution of methyl 2-[3-(2-chloropyrimidin-5-yl)-1,2-oxazol-5-yl]acetate (3 g, 11.828 mmol, 1 equiv) and NaOMe (1.92 g, 35.484 mmol, 3.00 equiv) in MeOH (50 mL) was stirred for 1 h at room temperature under an atmosphere of dry nitrogen. The mixture was acidified to pH 6 with HCl (aq.). The residue was dissolved in EtOAc (300 mL). The resulting mixture was washed with water (2×300 mL). The combined organic layers were washed with brine (1×300 mL) and dried over anhydrous Na₂SO₄. After filtration, the filtrate was concentrated under reduced pressure to afford [3-(2-methoxypyrimidin-5-yl)-1,2-oxazol-5-yl]acetic acid (2.5 g, crude product) as a light yellow solid which was used in the next step directly without further purification. LCMS (ESI) m/z: [M+H]⁺=236.

### Step 5: Preparation of methyl 2-[3-(2-methoxypyrimidin-5-yl)-1,2-oxazol-5-yl]acetate

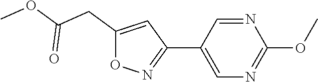

A solution of [3-(2-methoxypyrimidin-5-yl)-1,2-oxazol-5-yl]acetic acid (2.4 g, 10.204 mmol, 1 equiv) and (trimethylsilyl)diazomethane (2.33 g, 20.408 mmol, 2 equiv) in DCM (20 mL) and MeOH (5 mL) was stirred for 30 min at room temperature. The resulting mixture was concentrated under reduced pressure. The residue was purified by silica gel column chromatography, eluted with PE/EA (3:1) to afford methyl 2-[3-(2-methoxypyrimidin-5-yl)-1,2-oxazol-5-yl]acetate (1.2 g, 45.77%) as a white solid. LCMS (ESI) m/z: [M+H]⁺=250.

### Step 6: Preparation of methyl 2-[3-(2-methoxypyrimidin-5-yl)-1,2-oxazol-5-yl]-3-methylbutanoate

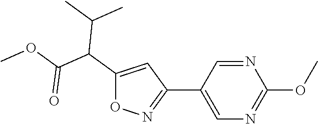

A solution of methyl 2-[3-(2-methoxypyrimidin-5-yl)-1,2-oxazol-5-yl]acetate (2.5 g, 10.031 mmol, 1 equiv) in THF (20 mL) was treated with t-BuOK (1.2 g, 10.694 mmol, 1.07 equiv) for 30 min at 0° C. under an atmosphere of dry nitrogen followed by the addition of 2-iodopropane (1.5 g, 8.824 mmol, 0.88 equiv) dropwise at 0° C. The resulting mixture was stirred for 12 h at room temperature. The mixture was acidified to pH 6 with HCl (aq.). The resulting mixture was extracted with EtOAc (2×200 mL). The combined organic layers were washed with brine (2×200 mL) and dried over anhydrous Na₂SO₄. After filtration, the filtrate was concentrated under reduced pressure. The residue was purified by silica gel column chromatography, eluted with PE/EA (3:1) to afford methyl 2-[3-(2-methoxypyrimidin-5-yl)-1,2-oxazol-5-yl]-3-methylbutanoate (310 mg, 10.08%) as a yellow oil. LCMS (ESI) m/z: [M+H]⁺=292.

### Step 7: Preparation of methyl 2-[3-(2-chloropyrimidin-5-yl)-1,2-oxazol-5-yl]-3-methylbutanoate

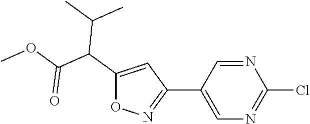 [SMILES: COC(=O)C(c1cc(-c2cnc(Cl)nc2)no1)C(C)C]

A solution of methyl 2-[3-(2-methoxypyrimidin-5-yl)-1,2-oxazol-5-yl]-3-methylbutanoate (200 mg, 0.687 mmol, 1 equiv) and POCl₃ (1.9 mL, 20.61 mmol, 30 equiv) in DMF (1.5 mL) was stirred for 3 h at 60° C. under an atmosphere of dry nitrogen. The residue was dissolved in EtOAc (100 mL). The resulting mixture was washed with brine (2×100 mL) and dried over anhydrous Na₂SO₄. After filtration, the filtrate was concentrated under reduced pressure to afford methyl 2-[3-(2-chloropyrimidin-5-yl)-1,2-oxazol-5-yl]-3-methylbutanoate (160 mg, crude product) as a brown oil which was used in the next step directly without further purification. LCMS (ESI) m/z: [M+H]⁺=296.

### Preparation of 2-((5-((R)-1-((2S,4R)-4-hydroxy-2-(((S)-1-(4-(2-methylthiazol-5-yl)phenyl)ethyl)carbamoyl)pyrrolidin-1-yl)-3-methyl-1-oxobutan-2-yl)isoxazol-3-yl)oxy)acetic acid (I-10

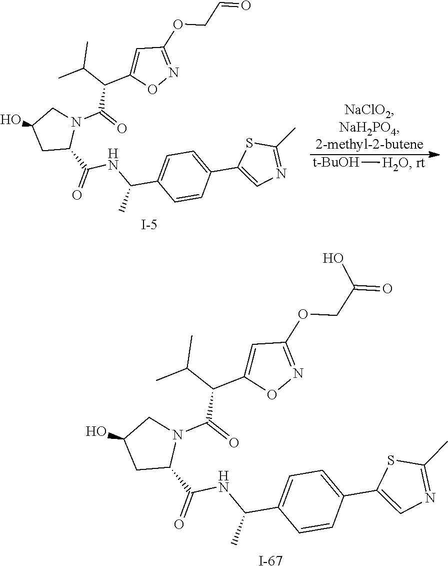

To a stirred solution of (2S,4R)-4-hydroxy-N-[(1S)-1-[4-(2-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (30.00 mg, 0.055 mmol, 1.00 equiv) and 2-methyl-2-butene (0.78 mg, 0.011 mmol, 0.20 equiv) in tert-butanol (2 mL) was added dropwise a solution of NaClO₂ (50.19 mg, 0.550 mmol, 10.00 equiv) and NaH₂PO₄ (78.77 mg, 0.550 mmol, 10.00 equiv) in water (2.00 mL) at 0° C. The mixture was stirred at 0° C. for 0.5 h, then warmed up to room temperature and stirred for 1.5 h. The reaction was quenched by addition of a mixture of saturated Na₂S₂O₃ solution and brine, extracted with CHCl₃ (20 mL×3). The combined organic extracts were dried over Na₂SO₄, filtered, concentrated in vacuo, and purified by silica gel chromatography (PE/EtOAc=1:1 to 1:3). This provided intermediate I-10 (15.80 mg, 49.93%) as a colorless oil. LCMS (ESI) m/z: [M+H]⁺=557.

### Preparation of (2S,4R)-4-hydroxy-1-[(2R)-2-[3-(2-{3-[3-(2-hydroxyphenyl)-5H-pyrrolo[3,2-c]pyridazin-6-yl]azetidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 1)

### Preparation of 2-(6-(azetidin-3-yl)-5H-pyrrolo[3,2-c]pyridazin-3-yl)phenol (Intermediate 5)

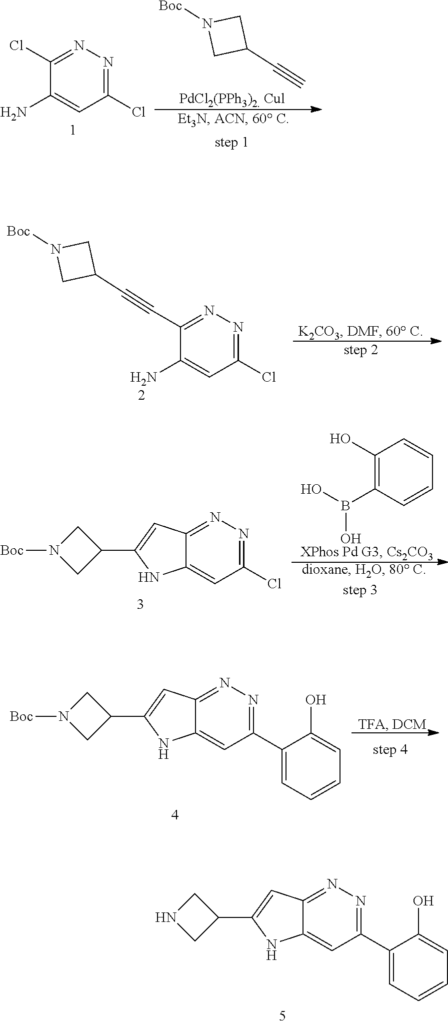

### Step 1: Preparation of tert-butyl 3-[2-(4-amino-6-chloropyridazin-3-yl)ethynyl]azetidine-1-carboxylate (Intermediate 2)

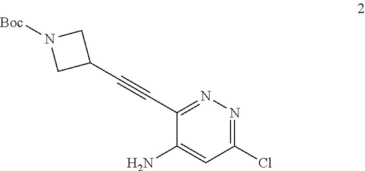 [SMILES: CC(C)(C)OC(=O)N1CC(C#Cc2nnc(Cl)cc2N)C1]

To a stirred solution of 3,6-dichloropyridazin-4-amine (2.87 g, 17.501 mmol, 1.00 equiv), Et₃N (8.85 g, 87.505 mmol, 5 equiv) and tert-butyl 3-ethynylazetidine-1-carboxylate (3.49 g, 19.251 mmol, 1.1 equiv) in ACN (45 mL) were added Pd(PPh₃)₂Cl₂ (2.46 g, 3.500 mmol, 0.2 equiv) and CuI (0.67 g, 3.500 mmol, 0.2 equiv). The resulting mixture was stirred for 2 h at 60° C. under nitrogen atmosphere. After concentration under reduced pressure, the residue was purified by flash chromatography with the following conditions: column, silica gel; mobile phase, EA in PE, 10% to 50% gradient in 25 min; detector, UV 254 nm. This resulted in intermediate 2 (2.87 g, 53.11%) as a brown yellow solid. LCMS (ESI) m/z [M+H]⁺=309.

### Step 2: Preparation of tert-butyl 3-{3-chloro-5H-pyrrolo[3,2-c]pyridazin-6-yl}azetidine-1-carboxylate (Intermediate 3)

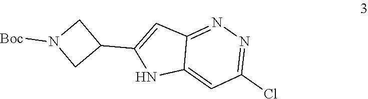 [SMILES: CC(C)(C)OC(=O)N1CC(c2cc3nnc(Cl)cc3[nH]2)C1]

A solution of intermediate 2 (2.82 g, 9.133 mmol, 1.00 equiv) and K₂CO₃ (3.79 g, 27.399 mmol, 3.00 equiv) in DMF (20 mL) was stirred for 2 h at 60° C. The mixture was purified by reverse phase flash chromatography with the following conditions: column, C18 silica gel; mobile phase, CH₃CN in water, 0% to 100% gradient in 25 min; detector, UV 254 nm. This resulted in intermediate 3 (1.27 g, 45.10%) as a yellow solid. LCMS (ESI) m/z: [M+H]⁺=309.

### Step 3: Preparation of tert-butyl 3-[3-(2-hydroxyphenyl)-5H-pyrrolo[3,2-c]pyridazin-6-yl]azetidine-1-carboxylate (Intermediate 4)

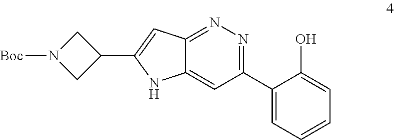 [SMILES: CC(C)(C)OC(=O)N1CC(c2cc3nnc(-c4ccccc4O)cc3[nH]2)C1]

A mixture of intermediate 3 (500 mg, 1.619 mmol, 1 equiv), 2-hydroxyphenylboronic acid (670.06 mg, 4.857 mmol, 3 equiv), XPhos Pd G3 (274.14 mg, 0.324 mmol, 0.2 equiv) and Cs₂CO₃ (1582.83 mg, 4.857 mmol, 3 equiv) in dioxane (5 mL) and H₂O (1 mL) was stirred for 2 h at 80° C. under nitrogen atmosphere. The resulting mixture was cooled down to room temperature and was extracted with EtOAc (3×100 mL). The combined organic layers were washed with brine (3×30 mL) then dried over anhydrous Na₂SO₄. After filtration, the filtrate was concentrated under reduced pressure to afford Intermediate 4 (702 mg) as a brown solid. LCMS (ESI) m/z: [M+H]⁺=367.

### Step 4: Preparation of 2-(6-(azetidin-3-yl)-5H-pyrrolo[3,2-c]pyridazin-3-yl)phenol (Intermediate 5)

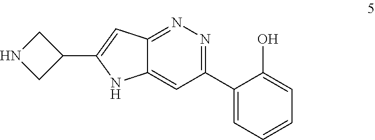 [SMILES: Oc1ccccc1-c1cc2[nH]c(C3CNC3)cc2nn1]

A mixture of Intermediate 4 (700 mg, 1.910 mmol, 1 equiv) and TFA (1.00 mL) in DCM (3 mL) was stirred for 2 h at room temperature. The resulting mixture was concentrated under reduced pressure to afford Intermediate 5 (150 mg, 29.48%) as a yellow solid. LCMS (ESI) m/z [M+H]⁺=267.

### Preparation of (2S,4R)-4-hydroxy-1-[(2R)-2-[3-(2-{3-[3-(2-hydroxyphenyl)-5H-pyrrolo[3,2-c]pyridazin-6-yl]azetidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 1)

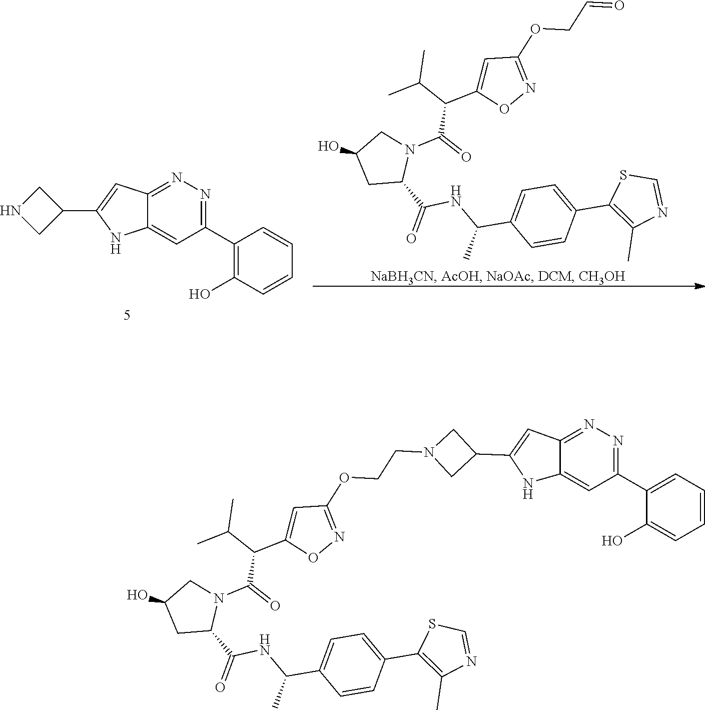 [SMILES: Oc1ccccc1-c1cc2[nH]c(C3CNC3)cc2nn1 ; Cc1ncsc1-c1ccc([C@H](C)NC(=O)[C@@H]2C[C@@H](O)CN2C(=O)[C@@H](c2cc(OCC=O)no2)C(C)C)cc1 ; Cc1ncsc1-c1ccc([C@H](C)NC(=O)[C@@H]2C[C@@H](O)CN2C(=O)[C@@H](c2cc(OCCN3CC(c4cc5nnc(-c6ccccc6O)cc5[nH]4)C3)no2)C(C)C)cc1]

A solution of Intermediate 5 (12.31 mg, 0.046 mmol, 1 equiv), (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (25 mg, 0.046 mmol, 1.00 equiv) and NaOAc (3.79 mg, 0.046 mmol, 1.0 equiv) in DCM (1 mL) and CH₃OH (1 mL) was stirred for 20 min at room temperature. To the above mixture was added NaBH₃CN (8.72 mg, 0.138 mmol, 3.0 equiv) and AcOH (cat.). The resulting mixture was stirred for 2 h at room temperature. The resulting mixture was concentrated under reduced pressure. The crude product was purified by Prep-HPLC with the following conditions: Column, Kinetex EVO C18 Column, 21.2*150, 5 m; Mobile Phase A: water (10 mmol/L NH₄HCO₃), Mobile Phase B: CH₃CN; Flow rate: 25 mL/min; Gradient: 37% B to 43% B in 7 min; Detector, UV 254/220 nm. This resulted in Compound 1 (11 mg, 29.50%) as a yellow solid. ¹H NMR (300 MHz, DMSO-d6) δ 14.47 (brs, 1H), 12.05 (brs, 1H), 8.98 (s, 1H), 8.42 (d, J=7.7 Hz, 1H), 8.25 (d, J=0.9 Hz, 1H), 8.04 (dt, J=8.3, 2.6 Hz, 1H), 7.48-7.40 (m, 2H), 7.40-7.26 (m, 3H), 7.01-6.92 (m, 2H), 6.84 (s, 1H), 6.09 (s, 1H), 5.10 (d, J=3.5 Hz, 1H), 4.92 (q, J=7.0 Hz, 1H), 4.37 (t, J=7.9 Hz, 1H), 4.28 (brs, 1H), 4.17 (t, J=5.4 Hz, 2H), 3.89 (q, J=7.2 Hz, 1H), 3.78-3.62 (m, 4H), 3.48-3.39 (m, 3H), 2.86 (t, J=5.4 Hz, 2H), 2.45 (d, J=2.2 Hz, 3H), 2.30-2.18 (m, 1H), 2.03 (t, J=9.8 Hz, 1H), 1.78 (ddd, J=12.7, 8.1, 4.7 Hz, 1H), 1.37 (d, J=7.0 Hz, 3H), 0.96 (d, J=6.4 Hz, 3H), 0.81 (dd, J=13.7, 6.7 Hz, 3H). LCMS (ESI) m/z: [M+H]⁺=791.30.

The compounds in Table 4 were prepared using procedures similar to those used above for the preparation of Compound 1 using the appropriate amine and aldehyde (or ketone).

TABLE 4

| | | LCMS (ESI) | |
|---|---|---|---|
| No. | Name | m/z | ¹H NMR |
| 2 | (2S,4R)-4-hydroxy-1-((R)-2-(3-((2-(3- | 804.30 | 1H NMR (400 MHz, Methanol-d4) δ 8.82 (d, |
| | (3-(2-hydroxyphenyl)-5H-pyrrolo[3,2- | | J = 34.5 Hz, 1H), 8.21 (s, 1H), 7.93 (d, J = |
| | c]pyridazin-6-yl)azetidin-1- | | 7.8 Hz, 1H), 7.45-7.22 (m, 5H), 6.98 (t, J = |
| | yl)ethyl)(methyl)amino)isoxazol-5-yl)-3- | | 7.4 Hz, 2H), 6.85 (s, 1H), 6.12 (s, 1H), |
| | methylbutanoyl)-N-((S)-1-(4-(4- | | 5.03 (d, J = 7.0 Hz, 1H), 4.57-4.35 (m, |
| | methylthiazol-5- | | 2H), 4.01-3.99 (m, 2H), 3.83 (m, 1H), 3.75- |
| | yl)phenyl)ethyl)pyrrolidine-2- | | 3.54 (m, 5H), 3.38 (s, 2H), 3.03-2.88 (m, |
| | carboxamide | | 4H), 2.42 (d, J = 37.1 Hz, 4H), 2.16 (t, J = |
| | | | 10.1 Hz, 1H), 2.06-1.86 (m, 1H), 1.52 (m, |
| | | | 3H), 1.28 (s, 1H), 1.03 (m, 3H), 0.87 (m, |
| | | | 3H). |
| 3 | (2S,4R)-4-hydroxy-1-((R)-2-(3-(3-(3-(3- | 804.95 | 1H NMR (400 MHz, DMSO-d6) δ 14.44- |
| | (2-hydroxyphenyl)-5H-pyrrolo[3,2- | | 14.10 (m, 1H), 12.27-12.00 (m, 1H), 10.10- |
| | c]pyridazin-6-yl)azetidin-1- | | 9.90 (m, 1H), 8.97 (s, 1H), 8.45-8.35 (m, |
| | yl)propoxy)isoxazol-5-yl)-3- | | 2H), 8.11-8.02 (m, 1H, FA), 7.43 (d, J = |
| | methylbutanoyl)-N-((S)-1-(4-(4- | | 8.1 Hz, 2H), 7.40-7.30 (m, 3H), 7.22- |
| | methylthiazol-5- | | 7.13 (m, 1H), 7.03-6.94 (m, 2H), 5.84 (s, |
| | yl)phenyl)ethyl)pyrrolidine-2- | | 1H), 5.20-5.10 (m, 1H), 4.99-4.82 (m, |
| | carboxamide, formic acid | | 1H), 4.62-4.43 (m, 3H), 4.43-4.20 (m, |
| | | | 4H), 3.94-3.82 (m, 2H), 3.72-3.64 (m, |
| | | | 1H), 3.63-3.54 (m, 1H), 3.54-3.46 (m, |
| | | | 1H), 2.45 (s, 3H), 2.44-2.38 (m, 1H), 2.31- |
| | | | 2.16 (m, 1H), 2.13-1.99 (m, 1H), 1.95- |
| | | | 1.72 (m, 4H), 1.35 (d, J = 6.9 Hz, 3H), 0.97 |
| | | | (d, J = 6.3 Hz, 3H), 0.89 (d, J = 6.3 Hz, 3H). |
| 4 | (2S,4R)-4-hydroxy-1-((R)-2-(3-(4-((3- | 844.20 | 1H NMR (400 MHz, DMSO-d6) δ 14.45 (s, 1H), |
| | (3-(2-hydroxyphenyl)-5H-pyrrolo[3,2- | | 12.04 (s, 1H), 8.99 (d, J = 2.3 Hz, 1H), 8.39 (d, |
| | c]pyridazin-6-yl)azetidin-1- | | J = 7.6 Hz, 1H), 8.26 (s, 1H), 8.09-8.03 (m, |
| | yl)methyl)piperidin-1-yl)isoxazol-5-yl)- | | 1H), 7.49-7.42 (m, 2H), 7.41-7.35 (m, 2H), |
| | 3-methylbutanoyl)-N-((S)-1-(4-(4- | | 7.34-7.28 (m, 1H), 7.01-6.94 (m, 2H), 6.86 |
| | methylthiazol-5- | | (s, 1H), 6.13 (s, 1H), 5.10 (d, J = 3.7 Hz, 1H), |
| | yl)phenyl)ethyl)pyrrolidine-2- | | 4.97-4.87 (m, 1H), 4.36 (t, J = 7.8 Hz, 1H), 4.29 |
| | carboxamide | | (s, 1H), 3.94-3.84 (m, 1H), 3.76-3.52 (m, 6H), |
| | | | 3.47-3.38 (m, 2H), 2.75 (t, J = 12.1 Hz, 2H), |
| | | | 2.48-2.35 (m, 5H), 2.29-2.13 (m, 2H), 2.07- |
| | | | 1.97 (m, 1H), 1.84-1.69 (m, 3H), 1.56-1.48 |
| | | | (m, 1H), 1.42 (dd, J = 30.3, 7.0 Hz, 3H), 1.26- |
| | | | 1.13 (m, 2H), 0.96 (d, J = 6.6 Hz, 3H), 0.81 (dd, |
| | | | J = 15.5, 6.6 Hz, 3H). |
| 5 | (2S,4R)-4-hydroxy-1-((R)-2-(3-(4-(3-(3- | 830.20 | 1H NMR (400 MHz, Methanol-d4) δ 8.87 (d, J = |
| | (2-hydroxyphenyl)-5H-pyrrolo[3,2- | | 3.0 Hz, 1H), 8.24 (d, J = 1.1 Hz, 1H), 7.92 (d, |
| | c]pyridazin-6-yl)azetidin-1-yl)piperidin- | | 1H), 7.48-7.37 (m, 4H), 7.34-7.28 (m, 1H), |
| | 1-yl)isoxazol-5-yl)-3-methylbutanoyl)- | | 7.03-6.92 (m, 3H), 6.12 (d, J = 20.2 Hz, 1H), |
| | N-((S)-1-(4-(4-methylthiazol-5- | | 5.08-5.00 (m, 1H), 4.51 (t, J = 8.2 Hz, 1H), 4.46- |
| | yl)phenyl)ethyl)pyrrolidine-2- | | 4.34 (m, 3H), 4.28-4.09 (m, 3H), 3.89-3.77 |
| | carboxamide | | (m, 3H), 3.68-3.57 (m, 2H), 3.20-3.11 (m, |
| | | | 1H), 2.95 (t, J = 12.2 Hz, 2H), 2.47 (d, J = 3.3 |
| | | | Hz, 3H), 2.41-2.32 (m, 1H), 2.22-2.14 (m, |
| | | | 1H), 2.09-1.93 (m, 3H), 1.56 (dd, J = 29.0, 7.0 |
| | | | Hz, 5H), 1.06 (dd, J = 6.9, 1.7 Hz, 3H), 0.90 (dd, |
| | | | J = 11.1, 6.7 Hz, 3H). |

### Preparation of (2S,4R)-4-hydroxy-1-[(2R)-2-[3-(4-{3-[3-(2-hydroxyphenyl)-5H-pyrrolo[3,2-c]pyridazin-6-yl]azetidine-1-carbonyl}piperidin-1-yl]-1,2-oxazol-5-yl]-3-methylbutanoyl-N-[(1S)-1-[4-(4-methyl-1,3-thiazol-5-yl)phenyl]ethyl]pyrrolidine-2-carboxamide (Compound 6)

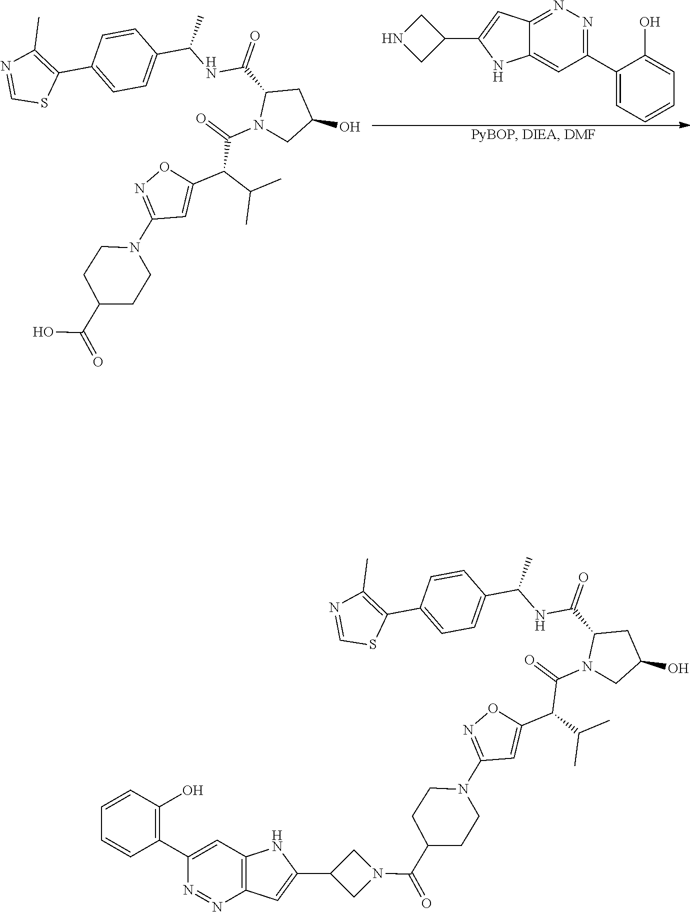 [SMILES: Cc1ncsc1-c1ccc([C@H](C)NC(=O)[C@@H]2C[C@@H](O)CN2C(=O)[C@@H](c2cc(N3CCC(C(=O)O)CC3)no2)C(C)C)cc1 ; Oc1ccccc1-c1cc2[nH]c(C3CNC3)cc2nn1 ; Cc1ncsc1-c1ccc([C@H](C)NC(=O)[C@@H]2C[C@@H](O)CN2C(=O)[C@@H](c2cc(N3CCC(C(=O)N4CC(c5cc6nnc(-c7ccccc7O)cc6[nH]5)C4)CC3)no2)C(C)C)cc1]

To a stirred solution of 1-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperidine-4-carboxylic acid (22.90 mg, 0.038 mmol, 1.00 equiv) in DMF (1.00 mL) were added PyBOP (58.62 mg, 0.114 mmol, 3.00 equiv) and DIEA (24.27 mg, 0.190 mmol, 5.00 equiv) at room temperature. To the above mixture was added Intermediate 5 (10.00 mg, 0.038 mmol, 1.00 equiv) at room temperature. The resulting mixture was stirred for 1 h at room temperature. The mixture was purified by Prep-HPLC with the following conditions: Column, Kinetex EVO C18 Column, 21.2*150, 5 μm; Mobile phase, water (10 mmol/L NH₄HCO₃) and MeOH (55% MeOH up to 77% in 7 min); Detector, UV 254/220 nm. This resulted in Compound 6 (8.0 mg, 24.11%) as a white solid. ¹H NMR (400 MHz, DMSO-d6) δ 14.39 (s, 1H), 12.24 (s, 1H), 8.99 (s, 1H), 8.41 (d, J=7.7 Hz, 1H), 8.29 (s, 1H), 8.07 (dd, J=8.4, 1.7 Hz, 1H), 7.49-7.41 (m, 2H), 7.41-7.34 (m, 2H), 7.34-7.28 (m, 1H), 7.03-6.93 (m, 3H), 6.16 (s, 1H), 5.11 (d, J=3.7 Hz, 1H), 4.97-4.86 (m, 1H), 4.66 (t, J=8.5 Hz, 1H), 4.48-4.24 (m, 4H), 4.23-4.12 (m, 1H), 4.12-4.03 (m, 1H), 3.76-3.62 (m, 3H), 3.58 (d, J=10.0 Hz, 1H), 3.50-3.39 (m, 2H), 2.91-2.75 (m, 2H), 2.46 (s, 3H), 2.30-2.11 (m, 1H), 2.08-1.97 (m, 1H), 1.85-1.66 (m, 3H), 1.66-1.50 (m, 2H), 1.38 (d, J=7.0 Hz, 3H), 0.96 (t, J=6.7 Hz, 3H), 0.82 (d, J=6.7 Hz, 3H). LCMS (ESI) m/z: [M+H]⁺=858.20.

The compound in Table 5 was prepared using a procedure similar to the one used above for the preparation of Compound 6 using the appropriate amine and carboxylic acid.

TABLE 5

| | | LCMS (ESI) | |
|---|---|---|---|
| No. | Name | m/z | ¹H NMR |
| 7 | (2S,4R)-4-hydroxy-1-((R)-2-(3-(2- | 805.15 | 1H NMR (400 MHz, DMSO-d6) δ 14.40 (s, 1H), |
| | (3-(3-(2-hydroxyphenyl)-5H- | | 12.01 (s, 1H), 8.98 (s, 1H), 8.46-8.39 (m, 1H), |
| | pyrrolo[3,2-c]pyridazin-6- | | 8.30 (s, 1H), 8.11-8.04 (m, 1H), 7.47-7.40 (m, |
| | yl)azetidin-1-yl)-2- | | 2H), 7.40-7.28 (m, 3H), 7.01-6.93 (m, 3H), |
| | oxoethoxy)isoxazol-5-yl)-3- | | 6.19 (d, J = 2.1 Hz, 1H), 5.12 (d, J = 3.7 Hz, 1H), |
| | methylbutanoyl)-N-((S)-1-(4-(4- | | 4.95-4.85 (m, 1H), 4.84-4.72 (m, 2H), 4.67 (t, |
| | methylthiazol-5- | | J = 8.7 Hz, 1H), 4.48-4.34 (m, 3H), 4.31-4.19 |
| | yl)phenyl)ethyl)pyrrolidine-2- | | (m, 2H), 4.22-4.12 (m, 1H), 3.75-3.65 (m, |
| | carboxamide | | 2H), 3.53-3.43 (m, 1H), 2.46 (s, 3H), 2.26- |
| | | | 2.21 (m, 1H), 2.10-1.98 (m, 1H), 1.84-1.73 |
| | | | (m, 1H), 1.47-1.31 (m, 3H), 0.99-0.93 (m, |
| | | | 3H), 0.87-0.76 (m, 3H). |

### Preparation of (2S,4R)-4-hydroxy-1-((R)-2-(3-(2-(3-(3-(2-hydroxyphenyl)-5-methyl-5H-pyrrolo[3,2-c]pyridazin-6-yl) azetidin-1-yl) ethoxy) isoxazol-5-yl)-3-methylbutanoyl)-N—((S)-1-(4-(4-methylthiazol-5-yl) phenyl) ethyl) pyrrolidine-2-carboxamide (Compound 8)

### Preparation of 2-(6-(azetidin-3-yl)-5-methyl-5H-pyrrolo[3,2-c]pyridazin-3-yl)phenol (Intermediate 7)

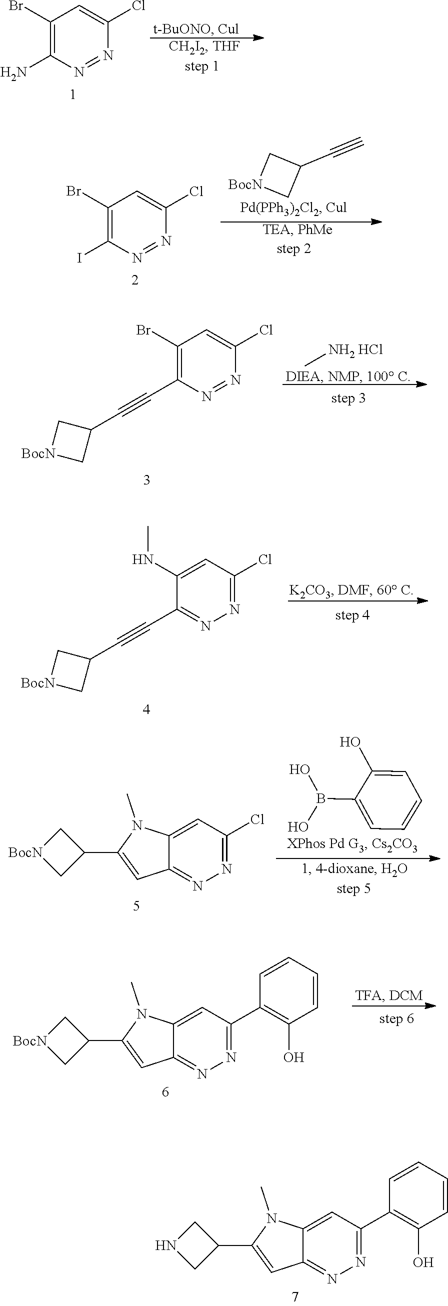

### Step 1: Preparation of 4-bromo-6-chloro-3-iodopyridazine (Intermediate 2)

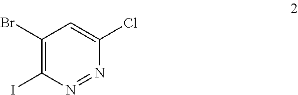

To a stirred mixture of 4-bromo-6-chloropyridazin-3-amine (10 g, 47.975 mmol, 1.00 equiv) and CuI (10.96 g, 57.570 mmol, 1.2 equiv) in THF (450 mL) were added CH₂I₂ (15.42 g, 57.570 mmol, 1.2 equiv) and t-BuONO (5.94 g, 57.570 mmol, 1.2 equiv) in portions at room temperature. The resulting mixture was stirred for 15 h at 60° C. The mixture was concentrated under reduced pressure and was purified by flash chromatography with the following conditions: column, silica gel; mobile phase, EtOAc in PE, 0% to 30% gradient in 20 min; detector, UV 254 nm. This resulted in Intermediate 2 (6.9 g, 45.04%) as a yellow solid. LCMS (ESI) m/z [M+H]⁺=319.

### Step 2: Preparation of tert-butyl 3-((4-bromo-6-chloropyridazin-3-yl) ethynyl) azetidine-1-carboxylate (Intermediate 3)

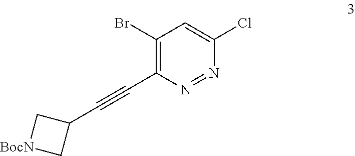 [SMILES: CC(C)(C)OC(=O)N1CC(C#Cc2nnc(Cl)cc2Br)C1]

To a stirred mixture of Intermediate 2 (1.6 g, 5.011 mmol, 1.00 equiv) and tert-butyl 3-ethynylazetidine-1-carboxylate (1.36 g, 7.517 mmol, 1.50 equiv) in toluene (10 mL) were added CuI (0.19 g, 1.002 mmol, 0.2 equiv) and Pd(PPh₃)₂Cl₂ (0.70 g, 1.002 mmol, 0.2 equiv). The resulting mixture was stirred for 2 h at 60° C. under nitrogen atmosphere. The mixture was concentrated under reduced pressure and the residue was purified by flash chromatography with the following conditions: column, silica gel; mobile phase, EtOAc in PE, 0% to 40% gradient in 10 min; detector, UV 254 nm. This resulted in Intermediate 3 (786 mg, 42.09%) as a yellow solid. LCMS (ESI) m/z: [M+H]⁺=372.

### Step 3: Preparation of tert-butyl 3-((6-chloro-4-(methylamino) pyridazin-3-yl) ethynyl) azetidine-1-carboxylate (Intermediate 4)

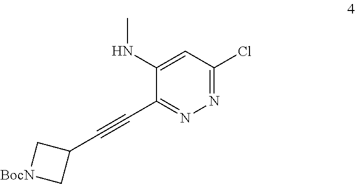 [SMILES: CNc1cc(Cl)nnc1C#CC1CN(C(=O)OC(C)(C)C)C1]

To a stirred mixture of Intermediate 3 (786 mg, 2.109 mmol, 1.00 equiv) and DIEA (0.82 g, 6.327 mmol, 3 equiv) in NMP (13 mL) was added methylamine hydrochloride (0.21 g, 3.163 mmol, 1.5 equiv). The resulting mixture was stirred for 2 h at 100° C. The mixture was purified by reverse phase flash chromatography with the following conditions: column, C18 silica gel; mobile phase, ACN in water, 0% to 100% gradient in 30 min; detector, UV 254 nm. This resulted in Intermediate 4 (598 mg, 87.83%) as a yellow solid. LCMS (ESI) m/z [M+H]⁺=323.

### Step 4: Preparation of tert-butyl 3-(3-chloro-5-methyl-5H-pyrrolo[3,2-c]pyridazin-6-yl) azetidine-1-carboxylate (Intermediate 5)

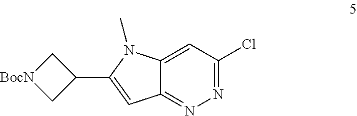 [SMILES: Cn1c(C2CN(C(=O)OC(C)(C)C)C2)cc2nnc(Cl)cc21]

To a stirred mixture of Intermediate 4 (420 mg, 0.620 mmol, 1.00 equiv) in DMF (2 mL) was added K₂CO₃ (256.90 mg, 1.860 mmol, 3 equiv). The resulting mixture was stirred for 2 h at 60° C. The mixture was purified by reverse phase flash chromatography with the following conditions: column, C18 silica gel; mobile phase, ACN in water, 0% to 100% gradient in 30 min; detector, UV 254 nm. This resulted in Intermediate 5 (164 mg, 39.05%) as a yellow solid. LCMS (ESI) m/z: [M+H]⁺=323.

### Step 5: Preparation of tert-butyl 3-(3-(2-hydroxyphenyl)-5-methyl-5H-pyrrolo[3,2-c]pyridazin-6-yl) azetidine-1-carboxylate (Intermediate 6)

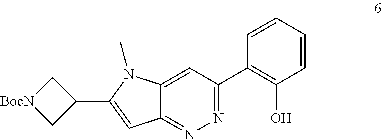 [SMILES: Cn1c(C2CN(C(=O)OC(C)(C)C)C2)cc2nnc(-c3ccccc3O)cc21]

To a stirred solution of Intermediate 5 (80 mg, 0.248 mmol, 1.00 equiv) and 2-hydroxyphenylboronic acid (102.55 mg, 0.744 mmol, 3 equiv) in dioxane (3.33 mL) and H₂O (0.67 mL) were added Cs₂CO₃ (242.25 mg, 0.744 mmol, 3 equiv) and XPhos Pd G3 (41.96 mg, 0.050 mmol, 0.20 equiv). The resulting mixture was stirred for 2 h at 80° C. under nitrogen atmosphere. The mixture was purified by reverse phase flash chromatography with the following conditions: column, C18 silica gel; mobile phase, ACN in water, 0% to 100% gradient in 30 min; detector, UV 254 nm. This resulted in Intermediate 6 (74.2 mg, 78.69%) as a yellow solid. LCMS (ESI) m/z: [M+H]⁺=381.

### Step 6: Preparation of 2-(6-(azetidin-3-yl)-5-methyl-5H-pyrrolo[3,2-c]pyridazin-3-yl)phenol (Intermediate 7)

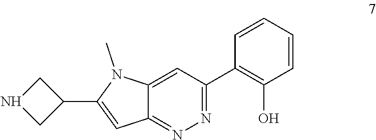 [SMILES: Cn1c(C2CNC2)cc2nnc(-c3ccccc3O)cc21]

A mixture of Intermediate 6 (74.8 mg, 0.197 mmol, 1.00 equiv) in TFA (1.5 mL) and DCM (0.5 mL) was stirred for 1 h at room temperature. The resulting mixture was concentrated under reduced pressure to afford Intermediate 7. The crude product was used in the next step directly without further purification. LCMS (ESI) m/z: [M+H]⁺=281.

### Preparation of (2S,4R)-4-hydroxy-1-((R)-2-(3-(2-(3-(3-(2-hydroxyphenyl)-5-methyl-5H-pyrrolo[3,2-c]pyridazin-6-yl) azetidin-1-yl) ethoxy) isoxazol-5-yl)-3-methylbutanoyl)-N—((S)-1-(4-(4-methylthiazol-5-yl) phenyl) ethyl) pyrrolidine-2-carboxamide (Compound 8)

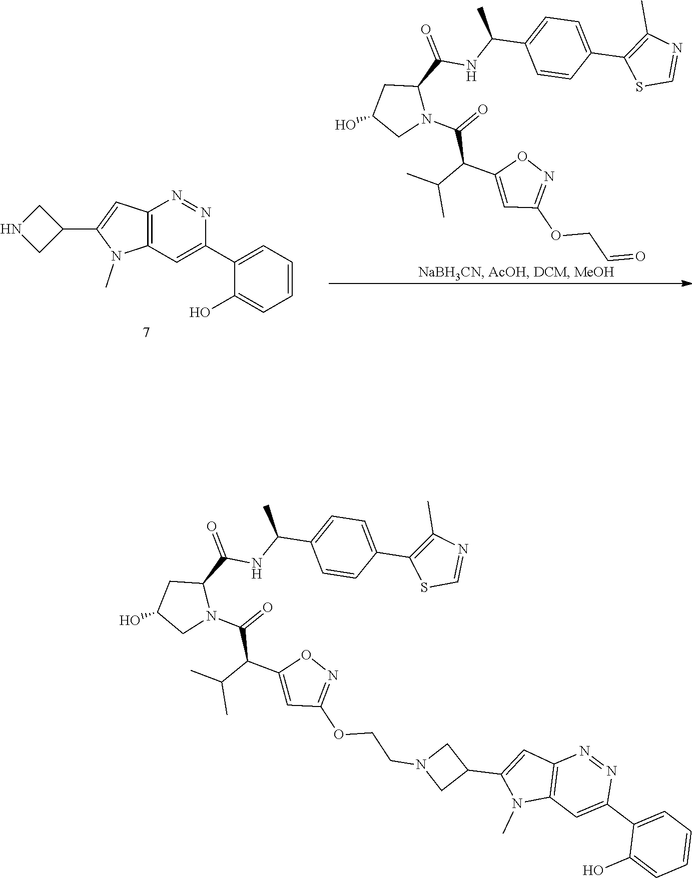 [SMILES: Cn1c(C2CNC2)cc2nnc(-c3ccccc3O)cc21 ; Cc1ncsc1-c1ccc([C@H](C)NC(=O)[C@@H]2C[C@@H](O)CN2C(=O)[C@@H](c2cc(OCC=O)no2)C(C)C)cc1 ; Cc1ncsc1-c1ccc([C@H](C)NC(=O)[C@@H]2C[C@@H](O)CN2C(=O)[C@@H](c2cc(OCCN3CC(c4cc5nnc(-c6ccccc6O)cc5n4C)C3)no2)C(C)C)cc1]

To a stirred mixture of (2S,4R)-4-hydroxy-N-[(1 S)-1-[4-(4-methyl-1,3-thiazol-5-yl) phenyl]ethyl]-1-[(2R)-3-methyl-2-[3-(2-oxoethoxy)-1,2-oxazol-5-yl]butanoyl]pyrrolidine-2-carboxamide (26 mg, 0.048 mmol, 1.00 equiv) and Intermediate 7 (18.74 mg, 0.067 mmol, 1.39 equiv) in DCM (1 mL) and MeOH (1 mL) was added AcOH (cat.). The mixture was stirred for 2 h at room temperature. To the mixture was added NaBH₃CN (9.07 mg, 0.144 mmol, 3 equiv). The resulting mixture was stirred for 2 h at room temperature. The mixture was purified by Prep-HPLC with the following conditions: Column, XBridge Prep C18 OBD Column, 19*150 mm, 5 μm; mobile phase, water (10 mmol/L NH₄HCO₃) and CH₃CN (34% CH₃CN up to 55% in 8 min). This resulted in Compound 8 (21.8 mg, 55.69%) as a white solid. ¹H NMR (300 MHz, DMSO-d6) δ 14.62 (s, 1H), 8.99 (s, 1H), 8.59 (s, 1H), 8.42 (d, J=7.6 Hz, 1H), 8.21-8.12 (m, 1H), 7.48-7.41 (m, 2H), 7.40-7.28 (m, 3H), 7.04-6.91 (m, 3H), 6.10 (s, 1H), 5.10 (d, J=3.7 Hz, 1H), 4.92 (t, J=7.1 Hz, 1H), 4.38 (t, J=7.8 Hz, 1H), 4.29 (s, 1H), 4.16 (t, J=5.3 Hz, 2H), 4.02 (t, J=7.6 Hz, 1H), 3.84 (t, J=7.2 Hz, 2H), 3.79-3.69 (m, 5H), 3.69-3.63 (m, 1H), 3.50-3.37 (m, 2H), 2.84 (t, J=5.5 Hz, 2H), 2.49-2.43 (m, 3H), 2.29-2.19 (m, 1H), 2.06-1.97 (m, 1H), 1.86-1.74 (m, 1H), 1.50-1.34 (m, 3H), 0.96 (d, J=6.4 Hz, 3H), 0.81 (d, J=6.7 Hz, 3H). LCMS (ESI) m/z: [M+H]⁺=805.34.

The compounds in Table 6 were prepared using procedures similar to those used above for the preparation of Compound 8 using the appropriate amine and aldehyde (or ketone).

TABLE 6

| | | LCMS (ESI) | |
|---|---|---|---|
| No. | Name | m/z | ¹H NMR |
| 9 | (2S,4R)-4-hydroxy-1-((R)-2-(3-(4- | 858.40 | 1H NMR (400 MHz, Methanol-d4) δ 8.87 (d, J = 4.1 |
| | ((3-(3-(2-hydroxyphenyl)-5-methyl- | | Hz, 1H), 8.37 (s, 1H), 8.03 (dd, J = 8.3, 1.6 Hz, 1H), |
| | 5H-pyrrolo[3,2-c]pyridazin-6- | | 7.48-7.35 (m, 4H), 7.35-7.27 (m, 1H), 7.04-6.92 (m, |
| | yl)azetidin-1-yl)methyl)piperidin-1- | | 3H), 6.10 (s, 1H), 5.03-5.00 (m, 1H), 4.58 (s, 3H), |
| | yl)isoxazol-5-yl)-3-methylbutanoyl)- | | 4.53-4.49 (m, 1H), 4.46-4.38 (m, 1H), 4.29-4.17 (m, |
| | N-((S)-1-(4-(4-methylthiazol-5- | | 2H), 3.88-3.70 (m, 7H), 3.64-3.59 (m, 2H), 2.93-2.82 |
| | yl)phenyl)ethyl)pyrrolidine-2- | | (m, 2H), 2.78 (s, 1H), 2.47 (d, J = 4.6 Hz, 3H), 2.42- |
| | carboxamide | | 2.32 (m, 1H), 2.23-2.13 (m, 1H), 2.02-1.90 (m, 1H), |
| | | | 1.87-1.67 (m, 3H), 1.60-1.51 (m, 3H), 1.37-1.30 (m, |
| | | | 2H), 1.05 (dd, J = 6.6, 2.2 Hz, 3H), 0.95-0.82 (m, 3H). |
| 10 | (2S,4R)-4-hydroxy-1-((R)-2-(3-((2- | 818.30 | 1H NMR (300 MHz, Methanol-d4) δ 8.96 (s, 1H), |
| | (3-(3-(2-hydroxyphenyl)-5-methyl- | | 8.44 (s, 1H), 8.12 (d, J = 8.2 Hz, 1H), 7.52 (s, 3H), |
| | 5H-pyrrolo[3,2-c]pyridazin-6- | | 7.45-7.32 (m, 2H), 7.13-7.04 (m, 2H), 7.02 (s, 1H), |
| | yl)azetidin-1- | | 6.21 (s, 1H), 5.13 (d, J = 7.0 Hz, 1H), 4.67 (t, J = 8.2 |
| | yl)ethyl)(methyl)amino)isoxazol-5- | | Hz, 1H), 4.52 (s, 1H), 4.17 (m, 3H), 3.93 (d, J = 6.8 |
| | yl)-3-methylbutanoyl)-N-((S)-1-(4- | | Hz, 1H), 3.85 (s, 2H), 3.80-3.68 (m, 4H), 3.46 (s, |
| | (4-methylthiazol-5- | | 2H), 3.05 (d, J = 8.0 Hz, 3H), 2.98-2.97 (m, 2H), |
| | yl)phenyl)ethyl)pyrrolidine-2- | | 2.56 (s, 3H), 2.48 (s, 2H), 2.25 (s, 1H), 2.10-1.96 |
| | carboxamide | | (m, 1H), 1.62 (dd, J = 18.3, 7.0 Hz, 3H), 1.15 (d, J = |
| | | | 6.5 Hz, 3H), 1.00 (t, J = 6.7 Hz, 3H). |
| 11 | (2S,4R)-4-hydroxy-1-((R)-2-(3-(3-(3- | 819.20 | 1H NMR (400 MHz, Methanol-d4) δ 8.86 (d, J = 12.7 |
| | (3-(2-hydroxyphenyl)-5-methyl-5H- | | Hz, 1H), 8.52 (d, J = 7.5 Hz, 1H), 7.84 (s, 1H), 7.48- |
| | pyrrolo[3,2-c]pyridazin-6-yl)azetidin- | | 7.33 (m, 6H), 7.07 (d, J = 6.1 Hz, 2H), 6.06 (s, 1H), |
| | 1-yl)propoxy)isoxazol-5-yl)-3- | | 5.08-4.99 (m, 1H), 4.84-4.72 (m, 1H), 4.59 (s, 3H), |
| | methylbutanoyl)-N-((S)-1-(4-(4- | | 4.50 (t, J = 8.2 Hz, 1H), 4.34 (t, J = 5.7 Hz, 2H), 3.89- |
| | methylthiazol-5- | | 3.81 (m, 4H), 3.78-3.67 (m, 1H), 3.60-3.55 (m, |
| | yl)phenyl)ethyl)pyrrolidine-2- | | 3H), 3.49-3.46 (m, 2H), 2.46 (d, J = 12.1 Hz, 3H), |
| | carboxamide | | 2.37 (s, 1H), 2.17 (d, J = 7.9 Hz, 3H), 2.01-1.91 (m, |
| | | | 1H), 1.55 (dd, J = 31.0, 7.0 Hz, 3H), 1.05 (d, J = 6.5 |
| | | | Hz, 3H), 0.94-0.86 (m, 3H). |
| 12 | (2S,4R)-4-hydroxy-1-((R)-2-(3-(4-(3- | 844.20 | 1H NMR (400 MHz, DMSO-d6) δ 14.62 (s, 1H), 8.99 |
| | (3-(2-hydroxyphenyl)-5-methyl-5H- | | (d, J = 1.8 Hz, 1H), 8.58 (s, 1H), 8.40 (d, J = 7.7 Hz, |
| | pyrrolo[3,2-c]pyridazin-6-yl)azetidin- | | 1H), 8.16 (dd, J = 8.4, 1.7 Hz, 1H), 7.50-7.41 (m, |
| | 1-yl)piperidin-1-yl)isoxazol-5-yl)-3- | | 2H), 7.37 (d, J = 8.4 Hz, 2H), 7.31 (ddd, J = 8.5, 7.2, |
| | methylbutanoyl)-N-((S)-1-(4-(4- | | 1.6 Hz, 1H), 7.02 - 6.94 (m, 2H), 6.91 (s, 1H), 6.14 |
| | methylthiazol-5- | | (s, 1H), 5.11 (d, J = 3.7 Hz, 1H), 4.92 (p, J = 7.0 Hz, |
| | yl)phenyl)ethyl)pyrrolidine-2- | | 1H), 4.37 (t, J = 7.8 Hz, 1H), 4.29 (s, 1H), 3.97 (p, |
| | carboxamide | | J = 7.6 Hz, 1H), 3.82-3.68 (m, 6H), 3.60-3.50 (m, |
| | | | 3H), 3.42 (d, J = 10.8 Hz, 1H), 3.28 (t, J = 7.1 Hz, |
| | | | 2H), 2.88 (t, J = 10.4 Hz, 2H), 2.46 (s, 3H), 2.31- |
| | | | 2.16 (m, 2H), 2.07-1.98 (m, 1H), 1.84-1.68 (m, |
| | | | 3H), 1.42 (dd, J = 29.8, 7.0 Hz, 3H), 1.25 (d, J = 10.5 |
| | | | Hz, 2H), 0.96 (t, J = 6.7 Hz, 3H), 0.81 (dd, J = 16.3, |
| | | | 6.6 Hz, 3H). |

### Preparation of (2S,4R)-4-hydroxy-1-[(2R)-2-[3-(4-{3-[3-(2-hydroxyphenyl)-5-methylpyrrolo[3,2-c]pyridazin-6-yl]azetidine-1-carbonyl} piperidin-1-yl)-1,2-oxazol-5-yl]-3-methylbutanoyl]-N-[(1S)-1-[4-(4-methyl-1,3-thiazol-5-yl) phenyl]ethyl]pyrrolidine-2-carboxamide (Compound 13)

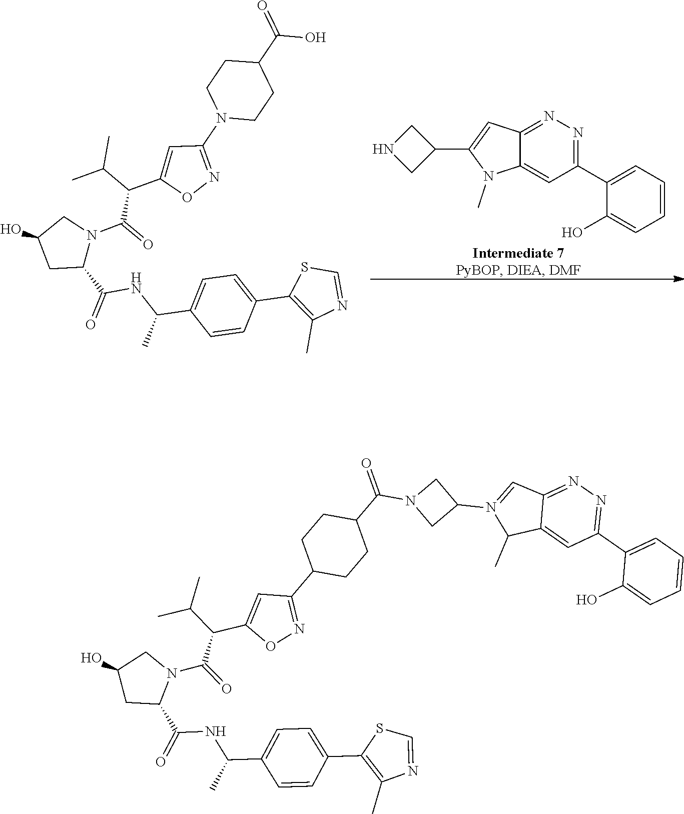

To a stirred mixture of 1-{5-[(2R)-1-[(2S,4R)-4-hydroxy-2-{[(1S)-1-[4-(4-methyl-1,3-thiazol-5-yl) phenyl]ethyl]carbamoyl}pyrrolidin-1-yl]-3-methyl-1-oxobutan-2-yl]-1,2-oxazol-3-yl}piperidine-4-carboxylic acid (20 mg, 0.033 mmol, 1 equiv) and Intermediate 7 (11.95 mg, 0.043 mmol, 1.3 equiv) in DMF (1 mL) were added PyBOP (34.14 mg, 0.066 mmol, 2 equiv) and DIEA (12.72 mg, 0.099 mmol, 3 equiv). The resulting mixture was stirred for 1.5 h at room temperature. The mixture was purified by Prep-HPLC with the following conditions: Column, SunFire Prep C188 OBD Column, 19*150 mm, 5 μm; mobile phase, water (0.1% NH₄HCO₃) and CH₃CN (13% CH₃CN up to 47% in 7 min). This resulted in Compound 13 (15.3 mg, 52.04%) as a white solid. ¹H NMR (400 MHz, DMSO-d6) δ14.56 (s, 1H), 8.98 (s, 1H), 8.62 (s, 1H), 8.40 (d, J=7.7 Hz, 1H), 8.21-8.14 (m, 1H), 7.50-7.41 (m, 2H), 7.40-7.29 (m, 3H), 7.14 (s, 1H), 7.03-6.95 (m, 2H), 6.16 (s, 1H), 5.11 (d, J=3.8 Hz, 1H), 4.91 (t, J=7.3 Hz, 1H), 4.74 (t, J=8.6 Hz, 1H), 4.52 (t, J=7.4 Hz, 1H), 4.43-4.33 (m, 2H), 4.31-4.26 (m, 2H), 4.15-4.07 (m, 1H), 3.77 (s, 3H), 3.75-3.63 (m, 3H), 3.58 (d, J=9.9 Hz, 1H), 3.44 (d, J=10.9 Hz, 1H), 2.82 (q, J=11.0 Hz, 2H), 2.46 (d, J=1.7 Hz, 3H), 2.24-2.20 (m, 2H), 2.04-1.97 (m, 1H), 1.86-1.64 (m, 3H), 1.56 (t, J=12.8 Hz, 2H), 1.38 (d, J=7.0 Hz, 3H), 1.00-0.92 (m, 3H), 0.82 (dd, J=15.2, 6.7 Hz, 3H). LCMS (ESI) m/z: [M+H]⁺=872.38.

The compound in Table 7 was prepared using a procedure similar to the one used above for the preparation of Compound 13 using the appropriate amine and carboxylic acid.

TABLE 7

| | | LCMS (ESI) | |
|---|---|---|---|
| No. | Name | m/z | ¹H NMR |
| 15 | (2S,4R)-4-hydroxy-1-((R)-2-(3-(2- | 819.20 | 1H NMR (400 MHz, DMSO-d6) δ 14.53 (s, 1H), |
| | (3-(3-(2-hydroxyphenyl)-5-methyl- | | 8.98 (s, 1H), 8.62 (s, 1H), 8.44 (t, J = 6.6 Hz, |
| | 5H-pyrrolo[3,2-c]pyridazin-6- | | 1H), 8.17 (dd, J = 8.5, 1.7 Hz, 1H), 7.44 (d, J = |
| | yl)azetidin-1-yl)-2- | | 8.0 Hz, 2H), 7.37 (d, J = 8.2 Hz, 2H), 7.37-7.28 |
| | oxoethoxy)isoxazol-5-yl)-3- | | (m, 1H), 7.13 (s, 1H), 7.03-6.95 (m, 2H), 6.19 |
| | methylbutanoyl)-N-((S)-1-(4-(4- | | (d, J = 2.0 Hz, 1H), 5.12 (d, J = 3.6 Hz, 1H), 4.90 |
| | methylthiazol-5- | | (q, J = 7.2 Hz, 1H), 4.81-4.64 (m, 3H), 4.55- |
| | yl)phenyl)ethyl)pyrrolidine-2- | | 4.32 (m, 4H), 4.29 (s, 1H), 4.19 (dd, J = 9.4, 6.0 |
| | carboxamide | | Hz, 1H), 3.77 (s, 3H), 3.70 (dd, J = 13.5, 10.0 |
| | | | Hz, 2H), 3.46 (d, J = 10.4 Hz, 1H), 2.45 (s, 3H), |
| | | | 2.28-2.19 (m, 1H), 2.04 (dd, J = 19.4, 8.5 Hz, |
| | | | 1H), 1.79 (ddd, J = 12.4, 7.6, 4.6 Hz, 1H), 1.47- |
| | | | 1.34 (m, 3H), 0.99-0.93 (m, 3H), 0.87-0.76 |
| | | | (m, 3H). |

### Preparation of (2S,4R)-1-((R)-2-(3-(2-(3-(5-(difluoromethyl)-3-(2-hydroxyphenyl)-5H-pyrrolo[3,2-c]pyridazin-6-yl)azetidin-1-yl)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide (Compound 14)

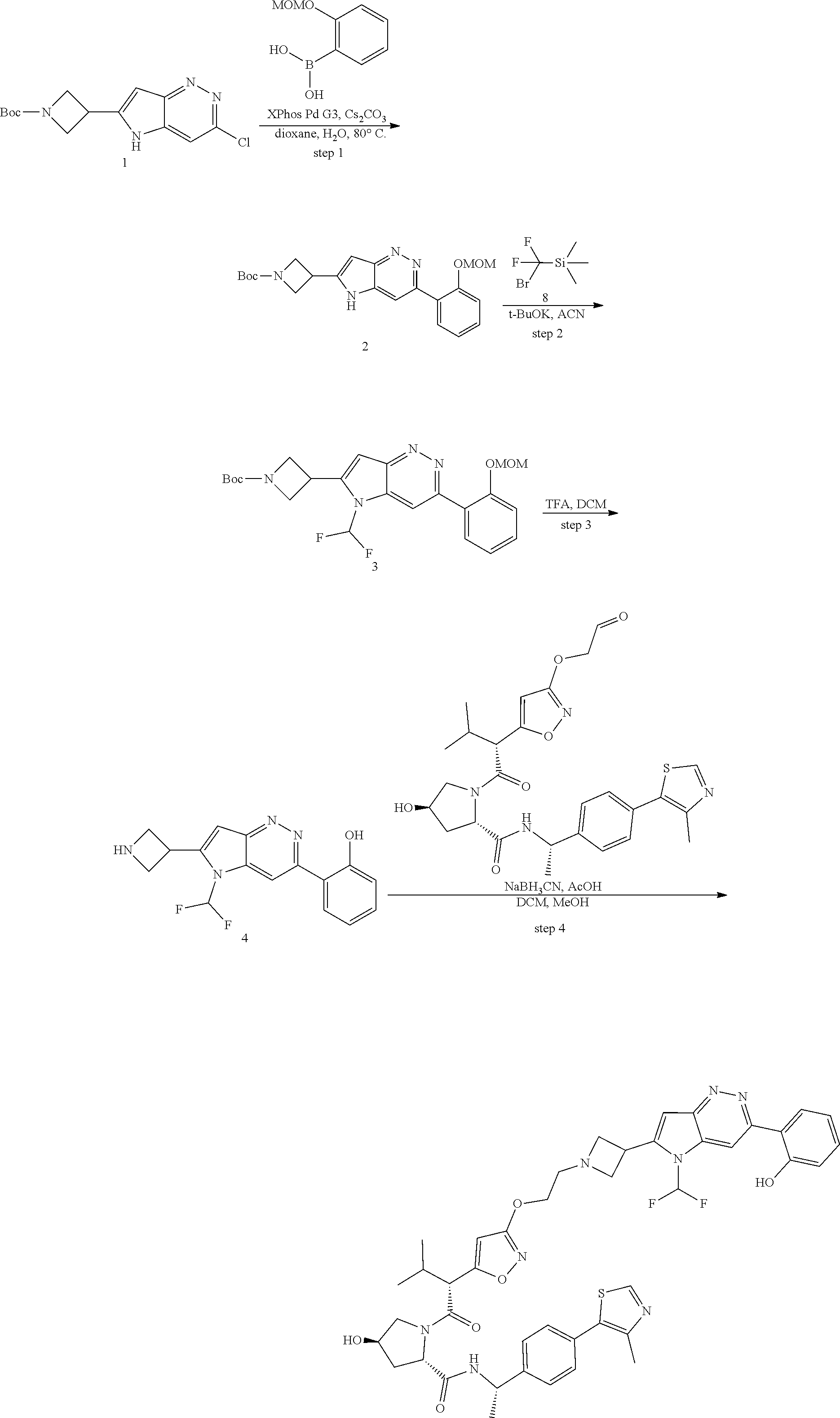

### Step 1: Preparation of tert-butyl 3-(3-(2-methoxymethoxy)phenyl)-5H-pyrrolo[3,2-c]pyridazin-6-yl)azetidine-1-carboxylate (Intermediate 2)

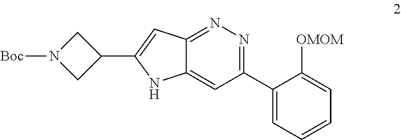

To a solution of tert-butyl 3-{3-chloro-5H-pyrrolo[3,2-c]pyridazin-6-yl}azetidine-1-carboxylate (500.00 mg, 1.619 mmol, 1 equiv) and 2-(methoxymethoxy)phenylboronic acid (442.03 mg, 2.429 mmol, 1.5 equiv) in dioxane (10.00 mL) and H₂O (2.00 mL) were added Cs₂CO₃ (1055.22 mg, 3.238 mmol, 2.0 equiv) and XPhos Pd G3 (137.07 mg, 0.162 mmol, 0.1 equiv). The resulting mixture was stirred overnight at 80° C. under nitrogen atmosphere. The resulting mixture was concentrated under reduced pressure. The residue was purified by reverse phase flash chromatography with the following conditions: column, C18 silica gel; mobile phase, CH₃CN in water, 0% to 100% gradient in 25 min; detector, UV 254 nm. This resulted in intermediate 2 (390 mg, 53.39%) as a brown yellow solid. LCMS (ESI) m/z: [M+H]⁺=411.5.

### Step 2: Preparation of tert-butyl 3-(5-(difluoromethyl)-3-(2-(methoxymethoxy)phenyl)-5H-pyrrolo[3,2-c]pyridazin-6-yl)azetidine-1-carboxylate (intermediate 3)

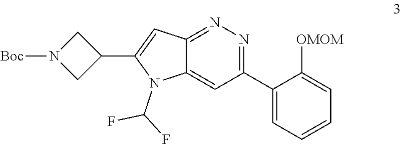

To a stirred mixture of intermediate 2 (120.00 mg, 0.292 mmol, 1 equiv) and (bromodifluoromethyl)trimethylsilane (29.69 mg, 0.146 mmol, 0.5 equiv) in CH₃CN (5.00 mL) was added t-BuOK (98.41 mg, 0.876 mmol, 3.0 equiv) at room temperature. The resulting mixture was stirred overnight at room temperature. The reaction was quenched with MeOH at room temperature. The resulting mixture was purified by reverse phase flash chromatography with the following conditions: column, C18 silica gel; mobile phase, MeOH in water, 0% to 100% gradient in 30 min; detector, UV 254 nm. This resulted in intermediate 3 (16 mg, 10.34%) as a brown oil. LCMS (ESI) m/z: [M+H]⁺=461.5.

### Step 3: Preparation of 2-(6-(azetidin-3-yl)-5-(difluoromethyl)-5H-pyrrolo[3,2-c]pyridazin-3-yl)phenol (intermediate 4)

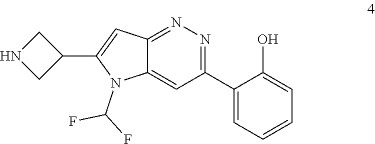 [SMILES: Oc1ccccc1-c1cc2c(cc(C3CNC3)n2C(F)F)nn1]

To a stirred solution of intermediate 3 (24.00 mg, 0.052 mmol, 1 equiv) in DCM (1.00 mL) was added TFA (1.00 mL) at room temperature. The resulting mixture was stirred for 1 h at room temperature. The resulting mixture was concentrated under reduced pressure. The residue was purified by reverse phase flash chromatography with the following conditions: column, C18 silica gel; mobile phase, MeOH in water, 0% to 100% gradient in 20 min; detector, UV 254 nm. This resulted in intermediate 4 (12 mg, 71.33%) as a yellow green oil. LCMS (ESI) m/z: [M+H]⁺=317.3.

### Step 4: Preparation of (2S,4R)-1-((R)-2-(3-(2-(3-(5-(difluoromethyl)-3-(2-hydroxyphenyl)-5H-pyrrolo[3,2-c]pyridazin-6-yl)azetidin-1-yl)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide (Compound 14)

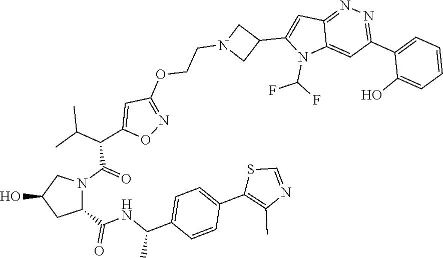 [SMILES: Cc1ncsc1-c1ccc([C@H](C)NC(=O)[C@@H]2C[C@@H](O)CN2C(=O)[C@@H](c2cc(OCCN3CC(c4cc5nnc(-c6ccccc6O)cc5n4C(F)F)C3)no2)C(C)C)cc1]

To a stirred mixture of intermediate 4 (10.00 mg, 0.032 mmol, 1 equiv) and (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (17.09 mg, 0.032 mmol, 1.0 equiv) in MeOH (1.00 mL) and DCM (1.00 mL) were added AcOH (cat.) and NaBH₃CN (5.96 mg, 0.096 mmol, 3.0 equiv) at room temperature. The resulting mixture was stirred overnight at room temperature. The resulting mixture was concentrated under reduced pressure. The residue was dissolved in DMF (1.00 mL) and was purified by Prep-HPLC with the following conditions: Column, XBridge Shield RP18 OBD, 19*150 mm, 5 μm; Mobile Phase A: water (10 mmol/L NH₄HCO₃), Mobile Phase B: CH₃CN; Flow rate: 25 mL/min; Gradient: 38% B to 60% B in 7 min; Detector, UV 254/220 nm. This resulted in Compound 14 (6.9 mg, 23.72%) as a white solid. ¹H NMR (400 MHz, DMSO-d6) δ 13.47 (s, 1H), 8.98 (d, J=1.7 Hz, 1H), 8.69 (s, 1H), 8.41 (d, J=7.7 Hz, 1H), 8.19-8.00 (m, 2H), 7.47-7.42 (m, 2H), 7.40-7.31 (m, 3H), 7.24 (s, 1H), 7.06-6.97 (m, 2H), 6.08 (d, J=10.5 Hz, 1H), 5.10 (d, J=3.7 Hz, 1H), 4.91 (t, J=7.1 Hz, 1H), 4.37 (t, J=7.9 Hz, 1H), 4.28 (s, 1H), 4.22-4.11 (m, 2H), 4.05 (t, J=7.4 Hz, 1H), 3.80 (t, J=7.2 Hz, 2H), 3.74-3.60 (m, 2H), 3.49-3.37 (m, 3H), 2.84 (t, J=5.3 Hz, 2H), 2.45 (d, J=3.0 Hz, 3H), 2.26-2.19 (m, 1H), 2.03 (t, J=9.9 Hz, 1H), 1.78 (ddd, J=12.7, 7.9, 4.7 Hz, 1H), 1.45-1.36 (m, 3H), 0.96 (d, J=6.5 Hz, 3H), 0.81 (dd, J=13.8, 6.6 Hz, 3H). LCMS (ESI) m/z: [M+H]⁺=841.3.

### Preparation of (2S,4R)-4-hydroxy-1-((R)-2-(3-(2-(3-(3-(2-hydroxyphenyl)-5-(oxetan-3-yl)-5H-pyrrolo[3,2-c]pyridazin-6-yl)azetidin-1-yl)ethoxy)isoxazol-5-yl)-3-methylbutanoyl)-N—((S)-1-(4-(4-methylthiazol-5-yl)phenyl)ethyl)pyrrolidine-2-carboxamide, formic acid (Compound 16)

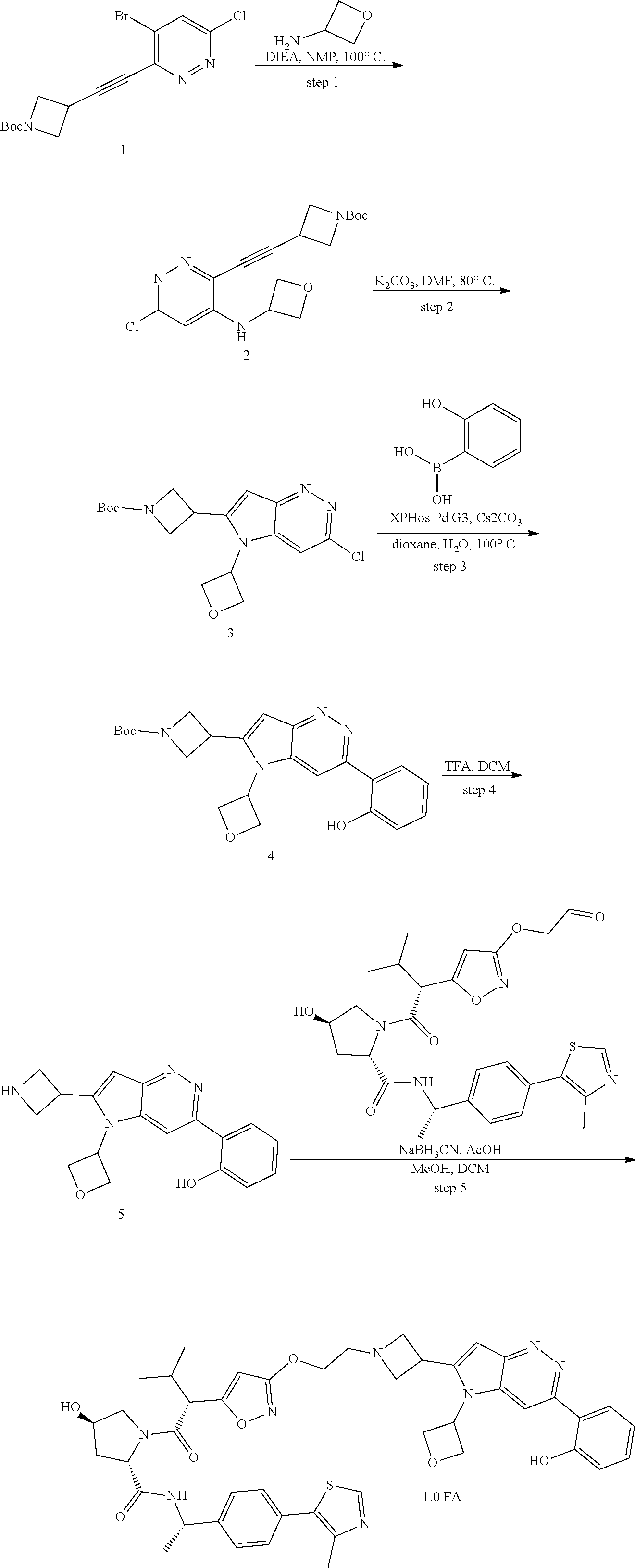

### Step 1: Preparation of tert-butyl 3-((6-chloro-4-(oxetan-3-ylamino)pyridazin-3-yl)ethynyl)azetidine-1-carboxylate (Intermediate 2)

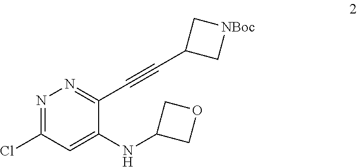 [SMILES: CC(C)(C)OC(=O)N1CC(C#Cc2nnc(Cl)cc2NC2COC2)C1]

A mixture of tert-butyl 3-((4-bromo-6-chloropyridazin-3-yl)ethynyl)azetidine-1-carboxylate (350 mg, 0.939 mmol, 1 equiv), oxetan-3-amine (102.98 mg, 1.408 mmol, 1.5 equiv) and DIEA (364.16 mg, 2.817 mmol, 3.0 equiv) in NMP (4 mL) was stirred for 2 h at 100° C. under nitrogen atmosphere. The resulting mixture was concentrated under reduced pressure. The residue was purified by silica gel column chromatography, eluted with PE/EA (5:1) to afford Intermediate 2 (300 mg, 87.55%) as a yellow solid. LCMS (ESI) m/z [M+H]⁺=365.

### Step 2: Preparation of tert-butyl 3-(3-chloro-5-(oxetan-3-yl)-5H-pyrrolo[3,2-c]pyridazin-6-yl)azetidine-1-carboxylate (Intermediate 3)

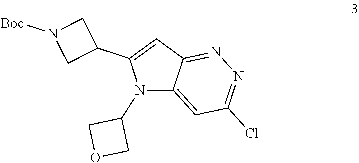 [SMILES: CC(C)(C)OC(=O)N1CC(c2cc3nnc(Cl)cc3n2C2COC2)C1]

A mixture of Intermediate 2 (170 mg, 0.466 mmol, 1 equiv) and K₂CO₃ (193.20 mg, 1.398 mmol, 3 equiv) in DMF (3 mL) was stirred overnight at 60° C. under nitrogen atmosphere. The resulting mixture was concentrated under reduced pressure. The residue was purified by reverse phase flash chromatography with the following conditions: column, C18 silica gel; mobile phase, MeOH in water, 10% to 50% gradient in 10 min; detector, UV 254 nm. This resulted in Intermediate 3 (150 mg, 88.24%) as a white solid. LCMS (ESI) m/z [M+H]⁺=365.

### Step 3: Preparation of tert-butyl 3-(3-(2-hydroxyphenyl)-5-(oxetan-3-yl)-5H-pyrrolo[3,2-c]pyridazin-6-yl)azetidine-1-carboxylate (Intermediate 4)

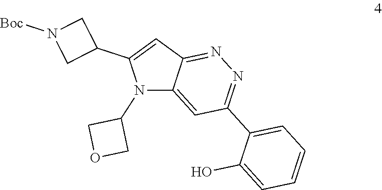 [SMILES: CC(C)(C)OC(=O)N1CC(c2cc3nnc(-c4ccccc4O)cc3n2C2COC2)C1]

A solution of Intermediate 3 (145 mg, 0.397 mmol, 1 equiv), 2-hydroxyphenylboronic acid (191.87 mg, 1.389 mmol, 3.5 equiv), XPhos Pd G3 (33.64 mg, 0.040 mmol, 0.1 equiv) and Cs₂CO₃ (388.49 mg, 1.191 mmol, 3 equiv) in dioxane (2.5 mL) and H₂O (0.5 mL) was stirred for 2 h at 100° C. under nitrogen atmosphere. The resulting mixture was concentrated under reduced pressure. The residue was purified by reverse phase flash chromatography with the following conditions: column, C18 silica gel; mobile phase, MeOH in water, 10% to 50% gradient in 10 min; detector, UV 254 nm. This resulted in Intermediate 4 (150 mg, 89.33%) as a black solid. LCMS (ESI) m/z [M+H]⁺=423.

### Step 4: Preparation of 2-(6-(azetidin-3-yl)-5-(oxetan-3-yl)-5H-pyrrolo[3,2-c]pyridazin-3-yl)phenol (Intermediate 5)

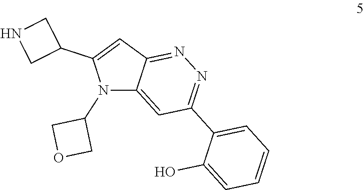 [SMILES: Oc1ccccc1-c1cc2c(cc(C3CNC3)n2C2COC2)nn1]

A solution of Intermediate 4 (150 mg, 0.355 mmol, 1 equiv) in TFA (1 mL) and DCM (4 mL) was stirred for 2 h at 0° C. under nitrogen atmosphere. The resulting mixture was concentrated under reduced pressure. This resulted in Intermediate 5 (330 mg, crude) as a black solid. LCMS (ESI) m/z [M+H]⁺=323.

### Step 5: Preparation of (2S,4R)-4-hydroxy-1-((R)-2-(3-(2-(3-(3-(2-hydroxyphenyl)-5-(oxetan-3-yl)-5H-pyrrolo[3,2-c]pyridazin-6-yl)azetidin-1-yl)ethoxy)isoxazol-5-yl)-3-methylbutanoyl)-N—((S)-1-(4-(4-methylthiazol-5-yl)phenyl)ethyl)pyrrolidine-2-carboxamide, formic acid (Compound 16)

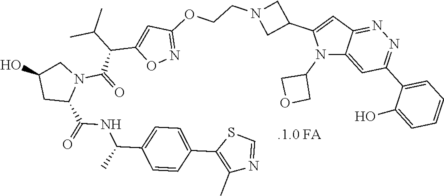 [SMILES: Cc1ncsc1-c1ccc([C@H](C)NC(=O)[C@@H]2C[C@@H](O)CN2C(=O)[C@@H](c2cc(OCCN3CC(c4cc5nnc(-c6ccccc6O)cc5n4C4COC4)C3)no2)C(C)C)cc1]

To a stirred solution of Intermediate 5 (13.88 mg, 0.230 mmol, 5 equiv) and (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (25 mg, 0.046 mmol, 1.00 equiv) in MeOH (1 mL) and DCM (1 mL) were added AcOH (one drop) and NaBH₃CN (7.26 mg, 0.115 mmol, 2.5 equiv). The resulting mixture was stirred overnight at room temperature under nitrogen atmosphere. The mixture was purified by Prep-HPLC. This resulted in Compound 16 (5.8 mg, 14.68%) as a white solid. 1H NMR (400 MHz, DMSO-d6) δ 13.89 (brs, 1H), 8.98 (s, 1H), 8.64 (s, 1H), 8.42 (d, J=7.5 Hz, 1H), 8.29 (FA salt, 1H), 7.98 (d, J=7.3 Hz, 1H), 7.44 (d, J=8.0 Hz, 2H), 7.34 (dd, J=15.9, 7.6 Hz, 3H), 7.05-6.97 (m, 3H), 6.09 (s, 1H), 5.54 (s, 1H), 5.18-5.05 (m, 5H), 4.92 (d, J=7.2 Hz, 1H), 4.37 (t, J=7.6 Hz, 1H), 4.28 (s, 1H), 4.15 (d, J=5.3 Hz, 2H), 3.98 (s, 1H), 3.80 (t, J=7.4 Hz, 3H), 3.65 (d, J=9.8 Hz, 2H), 2.82 (s, 2H), 2.45 (d, J=2.4 Hz, 2H), 2.25 (s, 3H), 2.03 (s, 1H), 1.45 (d, J=6.8 Hz, 1H), 1.38 (d, J=6.9 Hz, 1H), 1.15 (s, 3H), 0.96 (d, J=6.6 Hz, 3H), 0.81 (dd, J=13.6, 6.7 Hz, 3H). LCMS (ESI) m/z [M+H]⁺=847.40.

### Preparation of (2S,4R)-4-hydroxy-1-[(2R)-2-[3-(2-{4-[3-(2-hydroxyphenyl)-5H-pyrrolo[3,2-c]pyridazin-6-yl]piperidin-1-yL}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 17)

### Preparation of 2-[6-(piperidin-4-yl)-5H-pyrrolo[3,2-c]pyridazin-3-yl]phenol (Intermediate 5)

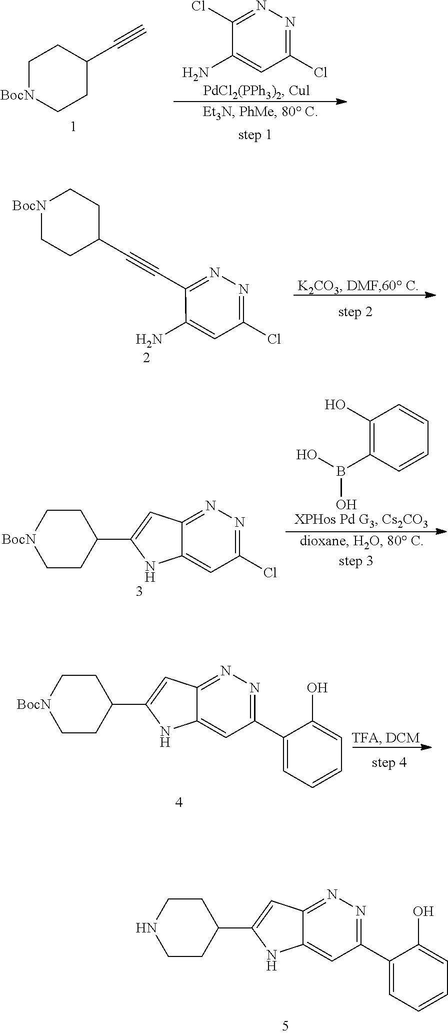

### Step 1: Preparation of tert-butyl 4-[(4-amino-6-chloropyridazin-3-yl)ethynyl]piperidine-1-carboxylate (Intermediate 2)

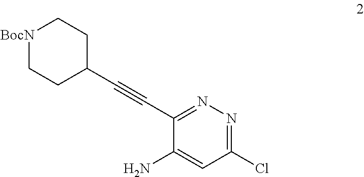 [SMILES: CC(C)(C)OC(=O)N1CCC(C#Cc2nnc(Cl)cc2N)CC1]

To a stirred mixture of 3,6-dichloropyridazin-4-amine (2 g, 12.196 mmol, 1 equiv) and tert-butyl 4-ethynylpiperidine-1-carboxylate (3.06 g, 14.635 mmol, 1.2 equiv) in toluene (30 mL) were added CuI (0.46 g, 2.439 mmol, 0.2 equiv) and Pd(PPh₃)₂Cl₂ (0.86 g, 1.220 mmol, 0.1 equiv) in portions at room temperature under nitrogen atmosphere. To the above mixture was added Et₃N (6.17 g, 60.980 mmol, 5.0 equiv) in portions over 2 min at room temperature. The resulting mixture was stirred for additional 3 h at 80° C. The mixture was allowed to cool down to room temperature. The resulting mixture was extracted with EtOAc (3×100 mL). The combined organic layers were washed with brine (3×30 mL) then dried over anhydrous Na₂SO₄. After filtration, the filtrate was concentrated under reduced pressure. The residue was purified by reverse phase flash chromatography with the following conditions: column, C18 silica gel; mobile phase, MeOH in water, 10% to 50% gradient in 10 min; detector, UV 254 nm. This resulted in Intermediate 2 (2.1 g, 51.12%) as a yellow solid. LCMS (ESI) m/z: [M+H]⁺=337.

### Step 2: Preparation of tert-butyl 4-{3-chloro-5H-pyrrolo[3,2-c]pyridazin-6-yl}piperidine-1-carboxylate (Intermediate 3)

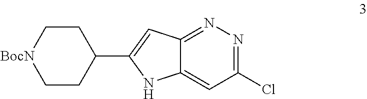 [SMILES: CC(C)(C)OC(=O)N1CCC(c2cc3nnc(Cl)cc3[nH]2)CC1]

A mixture of Intermediate 2 (1 g, 2.969 mmol, 1 equiv) and K₂CO₃ (2.05 g, 14.845 mmol, 5.0 equiv) in DMF (5 mL) was stirred for 24 h at 60° C. under nitrogen atmosphere. The mixture was allowed to cool down to room temperature. The resulting mixture was extracted with EtOAc (3×100 mL). The combined organic layers were washed with brine (3×30 mL) then dried over anhydrous Na₂SO₄. After filtration, the filtrate was concentrated under reduced pressure. The residue was purified by reverse phase flash chromatography with the following conditions: column, C18 silica gel; mobile phase, MeCN in water, 10% to 50% gradient in 10 min; detector, UV 254 nm. This resulted in Intermediate 3 (433 mg, 43.30%) as a yellow solid. LCMS (ESI) m/z: [M+H]⁺=337.

### Step 3: Preparation of tert-butyl 4-[3-(2-hydroxyphenyl)-5H-pyrrolo[3,2-c]pyridazin-6-yl]piperidine-1-carboxylate (Intermediate 4)

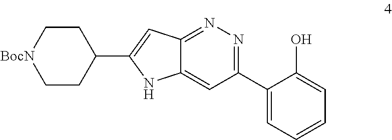 [SMILES: CC(C)(C)OC(=O)N1CCC(c2cc3nnc(-c4ccccc4O)cc3[nH]2)CC1]

To a stirred mixture of Intermediate 3 (200 mg, 0.594 mmol, 1 equiv) and 2-hydroxyphenylboronic acid (204.75 mg, 1.485 mmol, 2.5 equiv) in dioxane (5 mL) were added XPhos Pd G3 (100.52 mg, 0.119 mmol, 0.2 equiv) and Cs₂CO₃ (773.87 mg, 2.376 mmol, 4.0 equiv) in portions at room temperature under nitrogen atmosphere. To the above mixture was added H₂O (0.5 mL) dropwise at room temperature. The resulting mixture was stirred for additional 3 h at 80° C. The mixture was allowed to cool down to room temperature. The resulting mixture was extracted with EtOAc (3×100 mL). The combined organic layers were washed with brine (3×30 mL) then dried over anhydrous Na₂SO₄. After filtration, the filtrate was concentrated under reduced pressure. The residue was purified by reverse phase flash chromatography with the following conditions: column, C18 silica gel; mobile phase, MeCN in water, 10% to 50% gradient in 10 min; detector, UV 254 nm. This resulted in Intermediate 4 (130 mg, 55.50%) as a yellow solid. LCMS (ESI) m/z: [M+H]⁺=395.

### Step 4: Preparation of 2-[6-(piperidin-4-yl)-5H-pyrrolo[3,2-c]pyridazin-3-yl]phenol (Intermediate 5)

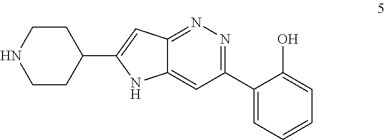 [SMILES: Oc1ccccc1-c1cc2[nH]c(C3CCNCC3)cc2nn1]

A mixture of Intermediate 4 (50 mg, 0.127 mmol, 1 equiv) and TFA (0.24 mL) in DCM (2 mL) was stirred for 1 h at room temperature. The resulting mixture was concentrated under reduced pressure. The crude product was used in the next step directly without further purification. LCMS (ESI) m/z: [M+H]⁺=295.

### Preparation of (2S,4R)-4-hydroxy-1-[(2R)-2-[3-(2-{4-[3-(2-hydroxyphenyl)-5H-pyrrolo[3,2-c]pyridazin-6-yl]piperidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 17)

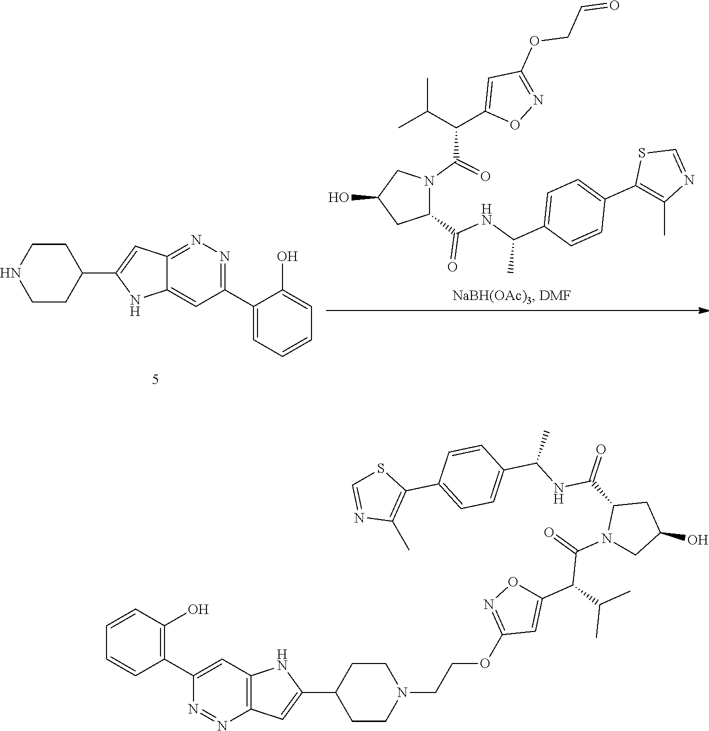 [SMILES: Oc1ccccc1-c1cc2[nH]c(C3CCNCC3)cc2nn1 ; Cc1ncsc1-c1ccc([C@H](C)NC(=O)[C@@H]2C[C@@H](O)CN2C(=O)[C@@H](c2cc(OCC=O)no2)C(C)C)cc1 ; Cc1ncsc1-c1ccc([C@H](C)NC(=O)[C@@H]2C[C@@H](O)CN2C(=O)[C@@H](c2cc(OCCN3CCC(c4cc5nnc(-c6ccccc6O)cc5[nH]4)CC3)no2)C(C)C)cc1]

A mixture of (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (20 mg, 0.037 mmol, 1 equiv) and intermediate 5 (13.07 mg, 0.044 mmol, 1.2 equiv) in DMF (0.6 mL) was stirred for 1 h at room temperature under nitrogen atmosphere. To the above mixture was added NaBH(OAc)₃ (23.52 mg, 0.111 mmol, 3.0 equiv) in portions over 1 min at room temperature. The resulting mixture was stirred for additional 2 h at room temperature. The mixture was purified by Prep-HPLC with the following conditions: Column, XBridge Shield RP18 OBD, 19*150 mm, 5 μm; Mobile Phase A: Water (10 mmol/L NH₄HCO₃), Mobile Phase B: CH₃CN; Flow rate: 25 mL/min; Gradient: 42% B to 55% B in 7 min; Detector, UV 254/220 nm. This resulted in Compound 17 (7.1 mg, 23.43%) as a white solid. 1H NMR (400 MHz, Methanol-d4) δ 8.85 (d, J=13.5 Hz, 1H), 8.18-8.10 (m, 1H), 7.95-7.88 (m, 1H), 7.46-7.27 (m, 5H), 7.03-6.94 (m, 2H), 6.65 (d, J=3.9 Hz, 1H), 6.01 (d, J=24.1 Hz, 1H), 5.05-5.00 (m, 1H), 4.60-4.51 (m, 1H), 4.44-4.39 (m, 3H), 3.84 (dd, J=10.8, 4.2 Hz, 1H), 3.77-3.66 (m, 1H), 3.62 (d, J=10.9 Hz, 1H), 3.17 (d, J=11.5 Hz, 2H), 2.93-2.85 (m, 3H), 2.48 (s, 3H), 2.36 (t, J=11.8 Hz, 3H), 2.14 (d, J=13.1 Hz, 3H), 1.95 (ddt, J=12.7, 8.9, 4.3 Hz, 3H), 1.55 (dd, J=31.9, 7.0 Hz, 3H), 1.06 (d, J=6.6 Hz, 3H), 0.91 (dd, J=9.2, 6.6 Hz, 3H). LCMS (ESI) m/z: [M+H]⁺=819.40.

### Preparation of (2S,4R)-4-hydroxy-1-[(2R)-2-[3-(2-{4-[3-(2-hydroxyphenyl)-5H-pyrrolo[3,2-c]pyridazin-6-yl]piperidin-1-yl)pyrimidin-5-yl}-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 18) and (2S,4R)-4-hydroxy-1-[(2S)-2-[3-(2-{4-[3-(2-hydroxyphenyl)-5H-pyrrolo[3,2-c]pyridazin-6-yl]piperidin-1-yl}pyrimidin-5-yl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 19)

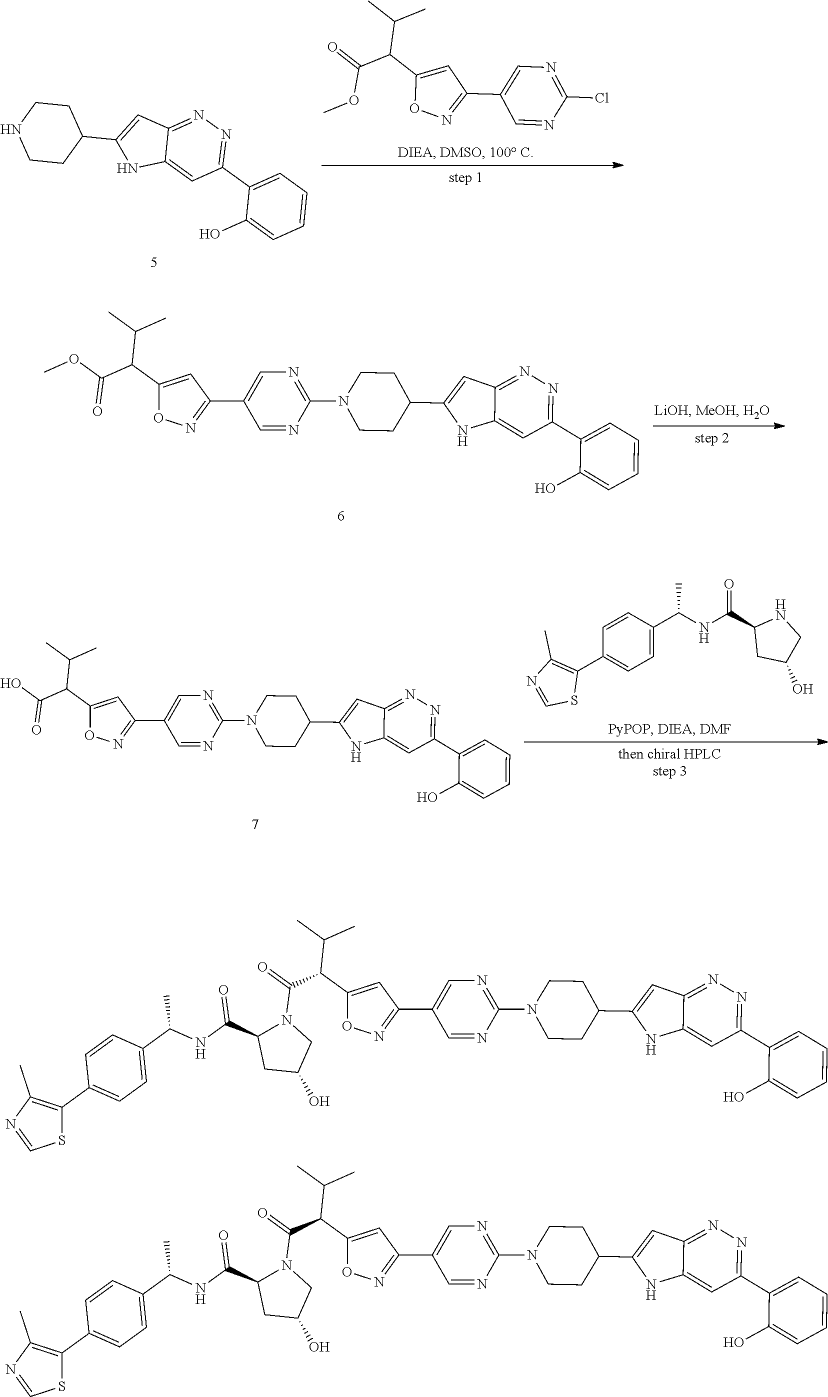

### Step 1: Preparation of methyl 2-[3-(2-{4-[3-(2-hydroxyphenyl)-5H-pyrrolo[3,2-c]pyridazin-6-yl]piperidin-1-yl}pyrimidin-5-yl)-1,2-oxazol-5-yl]-3-methylbutanoate (Intermediate 6)

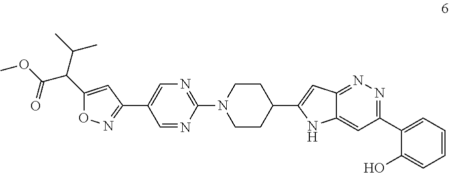 [SMILES: COC(=O)C(c1cc(-c2cnc(N3CCC(c4cc5nnc(-c6ccccc6O)cc5[nH]4)CC3)nc2)no1)C(C)C]

To a stirred solution of Intermediate 5 (500 mg, 1.70 mmol, 1 equiv) and methyl 2-[3-(2-chloropyrimidin-5-yl)-1,2-oxazol-5-yl]-3-methylbutanoate (503 mg, 1.70 mmol, 1.00 equiv) in DMSO (5 mL) was added DIEA (1.09 g, 8.50 mmol, 5.00 equiv). The resulting mixture was stirred for 1 h at 100° C. The mixture was allowed to cool down to room temperature and was purified by reverse phase flash chromatography with the following conditions: column, C18 silica gel; mobile phase, MeOH in water (0.1% FA), 0% to 100% gradient in 40 min; detector, UV 254 nm. This resulted in Intermediate 6 (200 mg, 42.54%) as a light yellow solid. LCMS (ESI) m/z: [M+H]⁺=554.

### Step 2: Preparation of 2-[3-(2-{4-[3-(2-hydroxyphenyl)-5H-pyrrolo[3,2-c]pyridazin-6-yl]piperidin-1-yl}pyrimidin-5-yl)-1,2-oxazol-5-yl]-3-methylbutanoic acid (Intermediate 7)

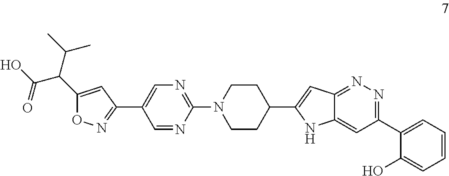 [SMILES: CC(C)C(C(=O)O)c1cc(-c2cnc(N3CCC(c4cc5nnc(-c6ccccc6O)cc5[nH]4)CC3)nc2)no1]

A solution of intermediate 6 (200 mg, 0.361 mmol, 1 equiv) and LiOH (86.52 mg, 3.610 mmol, 10 equiv) in MeOH (6 mL) and H2O (1.5 mL) was stirred for 2 h at 40° C. The mixture was acidified to pH 6 with HCl (aq.). The precipitated solid was collected by filtration and washed with water (3×2 mL). This resulted in intermediate 7 (102 mg, 52.33%) as a light yellow solid. LCMS (ESI) m/z: [M+H]⁺=540.

### Step 3: Preparation of (2S,4R)-4-hydroxy-1-[(2R)-2-[3-(2-{4-[3-(2-hydroxyphenyl)-5H-pyrrolo[3,2-c]pyridazin-6-yl]piperidin-1-yl}pyrimidin-5-yl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 18) and (2S,4R)-4-hydroxy-1-[(2S)-2-[3-(2-{4-[3-(2-hydroxyphenyl)-5H-pyrrolo[3,2-c]pyridazin-6-yl]piperidin-1-yl}pyrimidin-5-yl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 19)

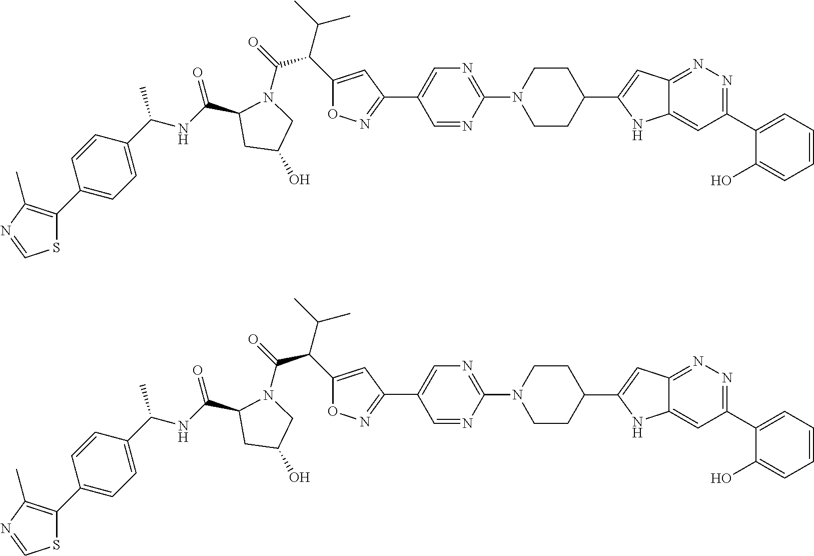 [SMILES: Cc1ncsc1-c1ccc([C@H](C)NC(=O)[C@@H]2C[C@@H](O)CN2C(=O)[C@@H](c2cc(-c3cnc(N4CCC(c5cc6nnc(-c7ccccc7O)cc6[nH]5)CC4)nc3)no2)C(C)C)cc1 ; Cc1ncsc1-c1ccc([C@H](C)NC(=O)[C@@H]2C[C@@H](O)CN2C(=O)[C@H](c2cc(-c3cnc(N4CCC(c5cc6nnc(-c7ccccc7O)cc6[nH]5)CC4)nc3)no2)C(C)C)cc1]

To a stirred solution of Intermediate 7 (68 mg, 0.126 mmol, 1 equiv) and (2S,4R)-4-hydroxy-N-[(1S)-1-[4-(4-methyl-1,3-thiazol-5-yl) phenyl]ethyl]pyrrolidine-2-carboxamide (45.94 mg, 0.139 mmol, 1.1 equiv) in DMF (2 mL) were added PyBOP (131.16 mg, 0.252 mmol, 2 equiv) and DIEA (48.86 mg, 0.378 mmol, 3 equiv). The resulting mixture was stirred for 2 h at room temperature. The mixture was purified by reverse phase flash chromatography with the following conditions: column, C18 silica gel; mobile phase, MeCN in water (10 mmol/L NH4HCO3), 10% to 80% gradient in 10 min; detector, UV 254 nm. The resulting residue was then purified by chiral HPLC with the following conditions: Column, CHIRALPAK ID, 2*25 cm, 5 μm; Mobile Phase A: MtBE (10 mM NH3-MeOH), Mobile Phase B: MeOH; Flow rate: 20 mL/min; Gradient: 20% B to 50% B in 15 min; Detector, UV 254/220 nm. This resulted in: Compound 18 (27.3 mg, 24.79%) as an off-white solid. 1H NMR (400 MHz, DMSO-d6) δ 14.49 (s, 1H), 12.07 (s, 1H), 8.92 (d, J=56.8 Hz, 3H), 8.44 (d, J=7.7 Hz, 1H), 8.23 (s, 1H), 8.05 (dd, J=8.5, 1.7 Hz, 1H), 7.49-7.34 (m, 4H), 7.30 (td, J=7.6, 1.6 Hz, 1H), 7.00-6.91 (m, 3H), 6.75 (d, J=2.4 Hz, 1H), 5.11 (d, J=3.6 Hz, 1H), 4.91 (t, J=9.7 Hz, 3H), 4.39 (t, J=7.9 Hz, 1H), 4.30 (s, 1H), 3.85 (d, J=9.8 Hz, 1H), 3.76 (dd, J=10.4, 4.4 Hz, 1H), 3.51 (d, J=10.6 Hz, 1H), 3.17 (t, J=12.5 Hz, 3H), 2.45 (d, J=6.6 Hz, 3H), 2.36-2.30 (m, 1H), 2.17 (d, J=12.7 Hz, 2H), 2.04 (t, J=10.6 Hz, 1H), 1.85-1.70 (m, 3H), 1.45-1.36 (m, 3H), 1.02 (dd, J=6.7, 4.2 Hz, 3H), 0.85 (dd, J=8.8, 5.9 Hz, 3H). LCMS (ESI) m/z: [M+H]⁺=853.35.

Compound 19 (13.5 mg, 12.52%) as an off-white solid. 1H NMR (400 MHz, DMSO-d6) δ 14.49 (d, J=3.8 Hz, 1H), 12.06 (s, 1H), 8.98 (d, J=18.7 Hz, 1H), 8.83 (d, J=18.0 Hz, 2H), 8.29 (d, J=7.9 Hz, 1H), 8.23 (s, 1H), 8.09-8.02 (m, 1H), 7.53-7.41 (m, 1H), 7.40-7.26 (m, 4H), 7.00-6.91 (m, 3H), 6.75 (s, 1H), 5.14 (d, J=3.6 Hz, 1H), 5.05-4.81 (m, 3H), 4.45 (t, J=7.7 Hz, 1H), 4.29 (s, 1H), 3.95 (d, J=9.0 Hz, 1H), 3.64-3.58 (m, 2H), 3.17 (t, J=12.4 Hz, 3H), 2.41 (s, 3H), 2.36-2.29 (m, 1H), 2.16 (d, J=12.5 Hz, 2H), 2.11-2.02 (m, 1H), 1.78 (dd, J=16.5, 8.9 Hz, 3H), 1.34 (d, J=7.0 Hz, 3H), 1.01 (d, J=6.6 Hz, 2H), 0.87 (t, J=6.4 Hz, 4H). LCMS (ESI) m/z: [M+H]⁺=853.35.

### Preparation of (2S,4R)-4-hydroxy-1-[(2R)-2-[3-(2-{4-[3-(2-hydroxyphenyl)-5-methylpyrrolo[3,2-c]pyridazin-6-yl]piperidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 20)

### Preparation of 2-(5-methyl-6-(piperidin-4-yl)-5H-pyrrolo[3,2-c]pyridazin-3-yl)phenol (Intermediate 4)

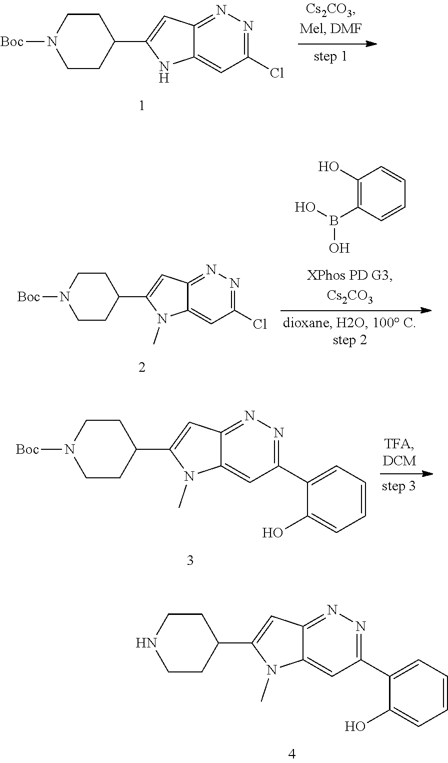 [SMILES: CC(C)(C)OC(=O)N1CCC(c2cc3nnc(Cl)cc3[nH]2)CC1 ; OB(O)c1ccccc1O ; Cn1c(C2CCN(C(=O)OC(C)(C)C)CC2)cc2nnc(Cl)cc21 ; Cn1c(C2CCN(C(=O)OC(C)(C)C)CC2)cc2nnc(-c3ccccc3O)cc21 ; Cn1c(C2CCNCC2)cc2nnc(-c3ccccc3O)cc21]

### Step 1: Preparation of tert-butyl 4-(3-chloro-5-methyl-5H-pyrrolo[3,2-c]pyridazin-6-yl)piperidine-1-carboxylate (Intermediate 2)

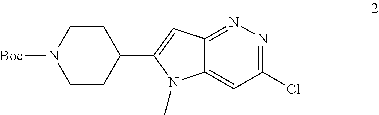 [SMILES: Cn1c(C2CCN(C(=O)OC(C)(C)C)CC2)cc2nnc(Cl)cc21]

To a stirred mixture of Intermediate 1 (380 mg, 1.128 mmol, 1 equiv) and Cs₂CO₃ (1.1 g, 3.384 mmol, 3 equiv) in DMF (4 mL) was added Mel (240.20 mg, 1.692 mmol, 1.5 equiv). The resulting mixture was stirred for 2 h at room temperature. The mixture was purified by reverse phase flash chromatography with the following conditions: column, C18 silica gel; mobile phase, CH₃CN in water, 0% to 50% gradient in 20 min; detector, UV 254 nm. This resulted in Intermediate 2 (196 mg, 49.52%) as a yellow green solid. LCMS (ESI) m/z [M+H]⁺=351.

### Step 2: Preparation of tert-butyl 4-(3-(2-hydroxyphenyl)-5-methyl-5H-pyrrolo[3,2-c]pyridazin-6-yl)piperidine-1-carboxylate (Intermediate 3)

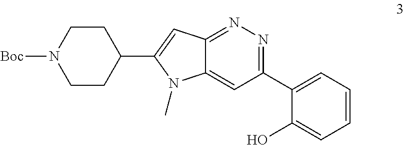 [SMILES: Cn1c(C2CCN(C(=O)OC(C)(C)C)CC2)cc2nnc(-c3ccccc3O)cc21]

To a stirred mixture of Intermediate 2 (180 mg, 0.513 mmol, 1 equiv) and 2-hydroxyphenylboronic acid (212.29 mg, 1.539 mmol, 3 equiv) in dioxane (4.17 mL) and H₂O (0.83 mL) were added XPhos Pd G3 (87.1 mg, 0.103 mmol, 0.2 equiv) and Cs₂CO₃ (501.48 mg, 1.539 mmol, 3 equiv). The resulting mixture was stirred for 1.5 h at 100° C. under nitrogen atmosphere. The mixture was purified by reverse phase flash chromatography with the following conditions: column, C18 silica gel; mobile phase, CH₃CN in water, 0% to 60% gradient in 23 min; detector, UV 254 nm. This resulted in Intermediate 3 (112 mg, 53.44%) as a yellow green solid. LCMS (ESI) m/z: [M+H]⁺=409.

### Step 3: Preparation of 2-(5-methyl-6-(piperidin-4-yl)-5H-pyrrolo[3,2-c]pyridazin-3-yl)phenol (Intermediate 4)

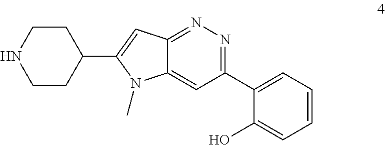 [SMILES: Cn1c(C2CCNCC2)cc2nnc(-c3ccccc3O)cc21]

A mixture of Intermediate 3 (112 mg, 0.274 mmol, 1 equiv) in TFA (3 mL) and DCM (1 mL) was stirred for 1 h at room temperature. The resulting mixture was concentrated under reduced pressure. This resulted in Intermediate 4 (159 mg, crude) as a brown yellow solid. LCMS (ESI) m/z: [M+H]⁺=309.

### Preparation of (2S,4R)-4-hydroxy-1-[(2R)-2-[3-(2-{4-[3-(2-hydroxyphenyl)-5-methylpyrrolo[3,2-c]pyridazin-6-yl]piperidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 20)

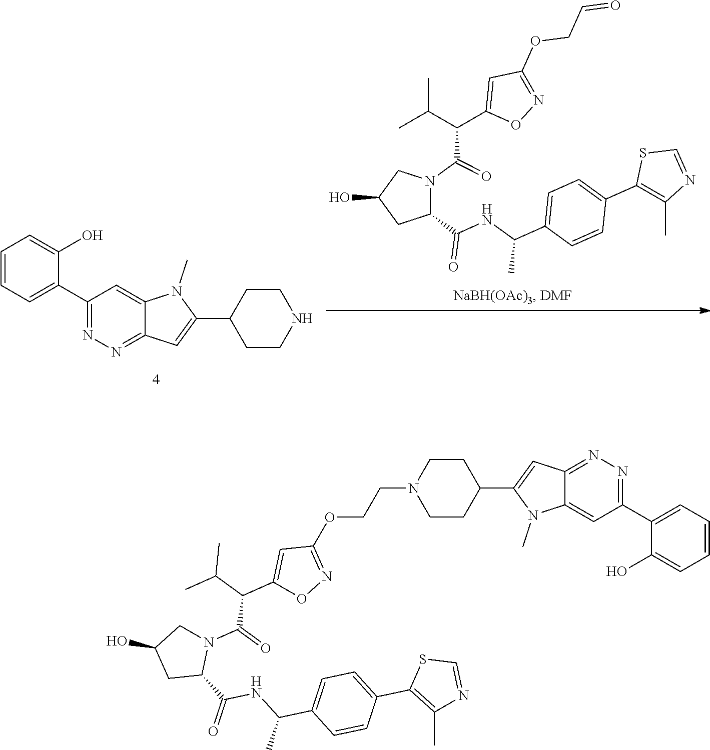 [SMILES: Cn1c(C2CCNCC2)cc2nnc(-c3ccccc3O)cc21 ; Cc1ncsc1-c1ccc([C@H](C)NC(=O)[C@@H]2C[C@@H](O)CN2C(=O)[C@@H](c2cc(OCC=O)no2)C(C)C)cc1 ; Cc1ncsc1-c1ccc([C@H](C)NC(=O)[C@@H]2C[C@@H](O)CN2C(=O)[C@@H](c2cc(OCCN3CCC(c4cc5nnc(-c6ccccc6O)cc5n4C)CC3)no2)C(C)C)cc1]

A mixture of intermediate 4 (15 mg, 0.049 mmol, 1 equiv) and (2S,4R)-4-hydroxy-N-[(1 S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (31.56 mg, 0.059 mmol, 1.2 equiv) in DMF (0.5 mL) was stirred for 1 h at room temperature. To the above mixture was added NaBH(OAc)₃ (30.93 mg, 0.147 mmol, 3 equiv) in portions at room temperature. The resulting mixture was stirred for additional 2 h at room temperature. The mixture was purified by Prep-HPLC with the following conditions: Column, XBridge Shield RP18 OBD, 19*150 mm, 5 μm; Mobile Phase A: water (10 mmol/L NH₄HCO₃), Mobile Phase B: CH₃CN; Flow rate: 25 mL/min; Gradient: 45% B to 70% B in 7 min; Detector, UV 254/220 nm. This resulted in Compound 20 (12.6 mg, 31.10%) as a white solid. ¹H NMR (400 MHz, Methanol-d4) δ 8.85 (d, J=15.2 Hz, 1H), 8.36-8.29 (m, 1H), 8.03 (d, J=8.2 Hz, 1H), 7.48-7.34 (m, 4H), 7.34-7.26 (m, 1H), 7.03-6.94 (m, 2H), 6.70 (d, J=3.6 Hz, 1H), 6.04 (s, 1H), 5.04-5.00 (m, 1H), 4.54-4.50 (m, 1H), 4.44-4.38 (m, 3H), 3.88 (s, 2H), 3.86-3.82 (m, 1H), 3.70-3.61 (m, 2H), 3.35 (s, 1H), 3.21-3.19 (d, J=11.1 Hz, 2H), 3.02-2.95 (m, 1H), 2.90-2.87 (m, 2H), 2.48-2.45 (m, 3H), 2.42-2.34 (m, 3H), 2.20-2.08 (m, 3H), 2.01-1.84 (m, 3H), 1.60-1.52 (m, 3H), 1.06 (d, J=6.5 Hz, 3H), 0.93-0.89 (m, 3H). LCMS (ESI) m/z: [M+H]⁺=833.3.

### Preparation of (2S,4R)-4-hydroxy-1-((R)-2-(3-(2-(4-(3-(2-hydroxyphenyl)-5-methyl-5H-pyrrolo[3,2-c]pyridazin-6-yl) piperidin-1-yl) pyrimidin-5-yl) isoxazol-5-yl)-3-methylbutanoyl)-N—((S)-1-(4-(4-methylthiazol-5-yl) phenyl) ethyl) pyrrolidine-2-carboxamide (Compound 21) and (2S,4R)-4-hydroxy-1-((S)-2-(3-(2-(4-(3-(2-hydroxyphenyl)-5-methyl-5H-pyrrolo[3,2-c]pyridazin-6-yl)piperidin-1-yl)pyrimidin-5-yl)isoxazol-5-yl)-3-methylbutanoyl)-N—((S)-1-(4-(4-methylthiazol-5-yl)phenyl)ethyl)pyrrolidine-2-carboxamide (Compound 22)

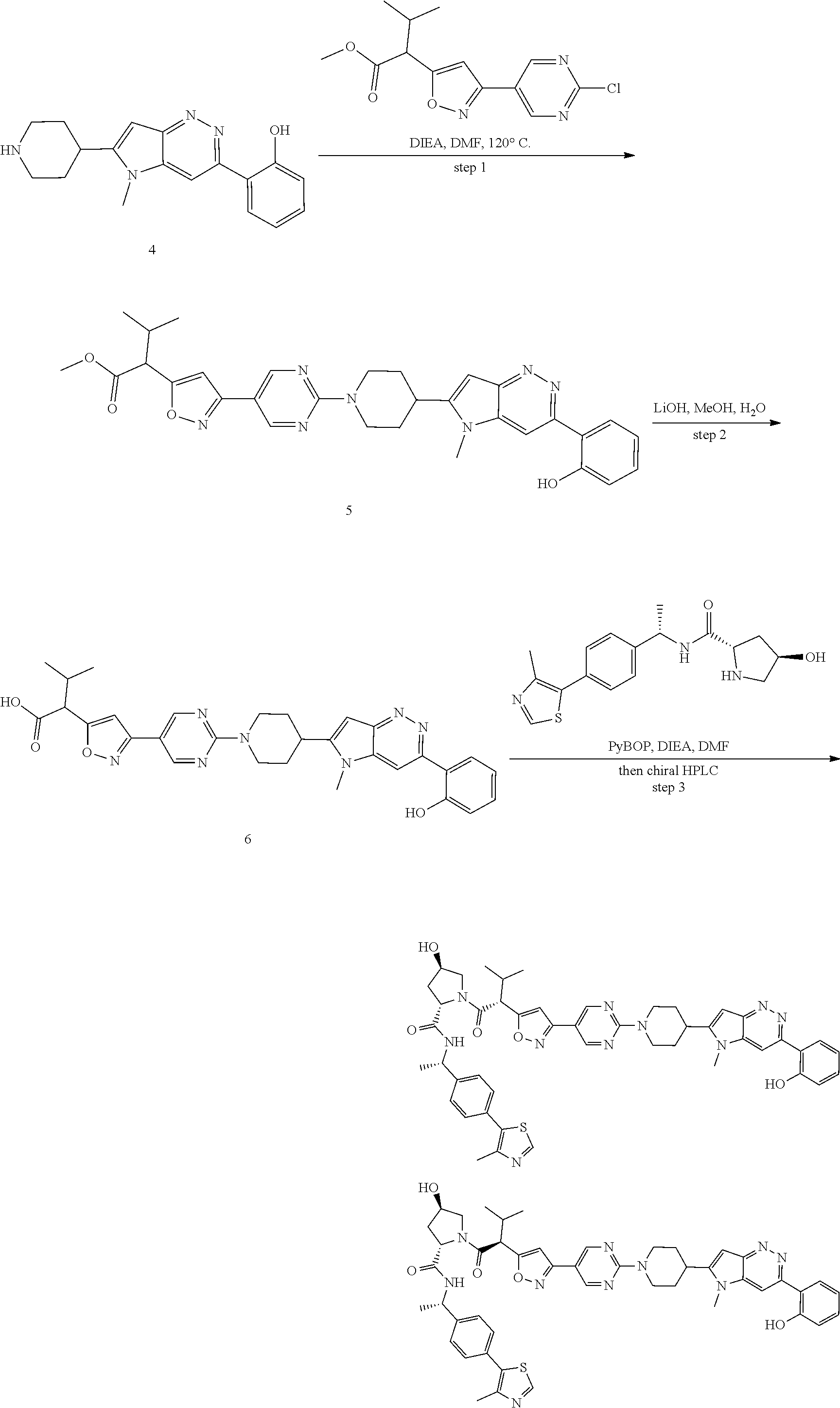

### Step 1: Preparation of methyl 2-(3-(2-(4-(3-(2-hydroxyphenyl)-5-methyl-5H-pyrrolo[3,2-c]pyridazin-6-yl) piperidin-1-yl) pyrimidin-5-yl) isoxazol-5-yl)-3-methylbutanoate (intermediate 5)

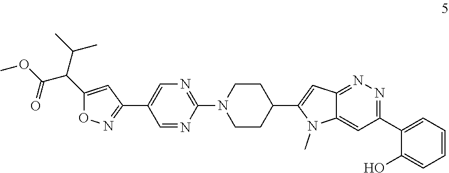 [SMILES: COC(=O)C(c1cc(-c2cnc(N3CCC(c4cc5nnc(-c6ccccc6O)cc5n4C)CC3)nc2)no1)C(C)C]

A solution of intermediate 4 (100 mg, 0.324 mmol, 1 equiv), methyl 2-[3-(2-chloropyrimidin-5-yl)-1,2-oxazol-5-yl]-3-methylbutanoate (86.80 mg, 0.324 mmol, 1 equiv) and DIEA (209 mg, 1.62 mmol, 5 equiv) in DMF (2 mL) was stirred for 2 h at 120° C. The resulting mixture was cooled down to room temperature and then was diluted with water (50 mL). The resulting mixture was extracted with EtOAc (2×100 mL). The combined organic layers were washed with brine (50 mL) then dried over anhydrous Na₂SO₄. After filtration, the filtrate was concentrated under reduced pressure. The residue was purified by reverse phase flash chromatography with the following conditions: column, C18 silica gel; mobile phase, CH₃CN in water, 0% to 50% gradient in 30 min; detector, UV 254 nm. This resulted in intermediate 5 (98 mg, 53.24%). LCMS (ESI) m/z: [M+H]⁺=568.

### Step 2: Preparation of 2-(3-(2-(4-(3-(2-hydroxyphenyl)-5-methyl-5H-pyrrolo[3,2-c]pyridazin-6-yl) piperidin-1-yl) pyrimidin-5-yl) isoxazol-5-yl)-3-methylbutanoic acid (intermediate 6)

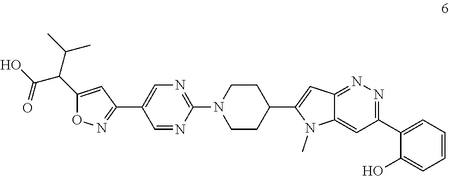 [SMILES: CC(C)C(C(=O)O)c1cc(-c2cnc(N3CCC(c4cc5nnc(-c6ccccc6O)cc5n4C)CC3)nc2)no1]

A solution of intermediate 5 (95 mg, 0.167 mmol, 1 equiv) and LiOH (8.02 mg, 0.334 mmol, 2 equiv) in H₂O (1 mL) and MeOH (1 mL) was stirred for 2 h at room temperature. The resulting mixture was extracted with EtOAc (3×50 mL). The combined organic layers were washed with brine (50 mL) then dried over anhydrous Na₂SO₄. After filtration, the filtrate was concentrated under reduced pressure. The residue was purified by reverse phase flash chromatography with the following conditions: column, C18 silica gel; mobile phase, CH₃CN in water (0.1% FA), 10% to 50% gradient in 25 min; detector, UV 254 nm. This resulted in intermediate 6 (76 mg, 82.03%). LCMS (ESI) m/z: [M+H]⁺=554.

### Step 3: Preparation of (2S,4R)-4-hydroxy-1-((R)-2-(3-(2-(4-(3-(2-hydroxyphenyl)-5-methyl-5H-pyrrolo[3,2-c]pyridazin-6-yl) piperidin-1-yl) pyrimidin-5-yl) isoxazol-5-yl)-3-methylbutanoyl)-N—((S)-1-(4-(4-methylthiazol-5-yl) phenyl) ethyl) pyrrolidine-2-carboxamide (Compound 21) and (2S,4R)-4-hydroxy-1-((S)-2-(3-(2-(4-(3-(2-hydroxyphenyl)-5-methyl-5H-pyrrolo[3,2-c]pyridazin-6-yl)piperidin-1-yl)pyrimidin-5-yl)isoxazol-5-yl)-3-methylbutanoyl)-N—((S)-1-(4-(4-methylthiazol-5-yl)phenyl)ethyl)pyrrolidine-2-carboxamide (Compound 22)

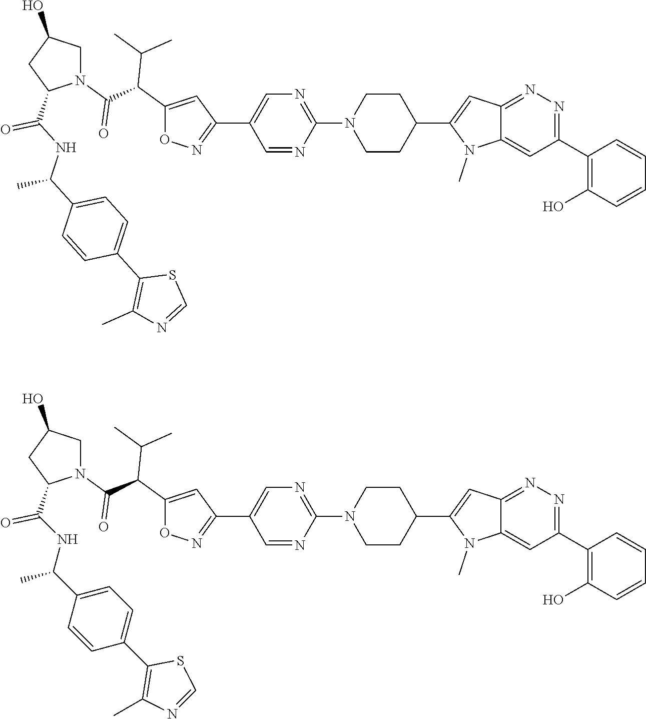 [SMILES: Cc1ncsc1-c1ccc([C@H](C)NC(=O)[C@@H]2C[C@@H](O)CN2C(=O)[C@@H](c2cc(-c3cnc(N4CCC(c5cc6nnc(-c7ccccc7O)cc6n5C)CC4)nc3)no2)C(C)C)cc1 ; Cc1ncsc1-c1ccc([C@H](C)NC(=O)[C@@H]2C[C@@H](O)CN2C(=O)[C@H](c2cc(-c3cnc(N4CCC(c5cc6nnc(-c7ccccc7O)cc6n5C)CC4)nc3)no2)C(C)C)cc1]

A solution of intermediate 6 (76 mg, 0.153 mmol, 1 equiv) in DMF (1 mL) was treated with (2S,4R)-4-hydroxy-N-[(1S)-1-[4-(4-methyl-1,3-thiazol-5-yl)phenyl]ethyl]pyrrolidine-2-carboxamide (96.08 mg, 0.289 mmol, 1.9 equiv), PyBOP (142.4 mg, 0.274 mmol, 1.8 equiv) and DIEA (49.96 mg, 0.386 mmol, 2.5 equiv) at room temperature. The mixture was stirred for 2 h at room temperature. The mixture was purified by reverse phase flash chromatography with the following conditions: Column, XBridge Shield RP18 OBD, 19*150 mm, 5 μm; Mobile Phase A: water (10 mmol/L NH₄HCO₃), Mobile Phase B: CH₃CN; Flow rate: 25 mL/min; Gradient: 46% B to 63% B in 7 min, then 63% B; Detector, UV 254/220 nm. The resulting residue was then purified by chiral HPLC with the following conditions: Column, CHIRALPAK ID-3, 4.6*50 mm, 3 μm; Mobile Phase A: MtBE (0.1% DIEA), Mobile Phase B: MeOH; Flow rate: 1.67 mL/min; Gradient: 20% B to 50% B. This resulted in:

Compound 21 (10.3 mg, 6.07%) as a white solid. ¹H NMR (300 MHz, DMSO-d6) δ 14.70 (s, 1H), 9.06 (s, 1H), 8.92 (d, J=2.3 Hz, 2H), 8.68 (s, 1H), 8.51-8.41 (m, 1H), 8.23-8.07 (m, 1H), 7.55-7.32 (m, 5H), 6.92 (s, 2H), 6.85 (s, 1H), 5.19 (d, J=3.6 Hz, 1H), 5.02-4.82 (m, 3H), 4.52-4.21 (m, 2H), 4.99 (s, 3H), 3.88-3.85 (m, 1H), 3.80-3.70 (m, 1H), 3.58-3.45 (m, 1H), 3.23 (d, J=12.4 Hz, 2H), 2.53 (d, J=5.0 Hz, 3H), 2.41-2.23 (m, 1H), 2.22-2.09 (m, 2H), 2.11 (t, J=10.6 Hz, 1H), 1.93-1.65 (m, 3H), 1.56-1.53 (m, 1H), 1.41-1.35 (m, 3H), 1.15 (s, 1H), 1.09 (d, J=6.4 Hz, 3H). LCMS (ESI) m/z: [M+H]⁺=867.3

Compound 22 (7.9 mg, 4.66%) as a white solid. ¹H NMR (300 MHz, DMSO-d6) δ 14.70 (s, 1H), 9.06-8.91 (m, 1H), 8.90 (d, J=14.5 Hz, 2H), 8.69 (s, 1H), 8.38-8.06 (m, 2H), 7.59-7.45 (m, 1H), 7.43-7.28 (m, 4H), 7.06-6.95 (m, 2H), 6.85 (s, 1H), 5.21 (s, 1H), 5.03-4.80 (m, 3H), 4.7-3.48 (m, 1H), 4.37 (s, 1H), 4.03 (s, 4H), 3.70-3.60 (s, 1H), 3.57-3.50 (m, 1H) 3.23-3.15 (m, 2H), 2.42-2.39 (m, 3H), 2.23-2.01 (m, 3H), 1.91-1.65 (m, 3H), 1.52-1.48 (m, 1H), 1.45-1.41 (m, 1H), 1.41-1.31 (m, 2H), 1.28-1.21 (m, 1H), 1.09 (d, J=6.6 Hz, 3H), 0.91-0.81 (m, 4H). LCMS (ESI) m/z: [M+H]⁺=867.3

### Preparation of (2S,4R)-1-[(2R)-2-[3-(2-{4-[5-ethyl-3-(2-hydroxyphenyl)pyrrolo[3,2-c]pyridazin-6-yl]piperidin-1-yl}pyrimidin-5-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 23) and (2S,4R)-1-[(2S)-2-[3-(2-{4-[5-ethyl-3-(2-hydroxyphenyl)pyrrolo[3,2-c]pyridazin-6-yl]piperidin-1-yl}pyrimidin-5-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 24)

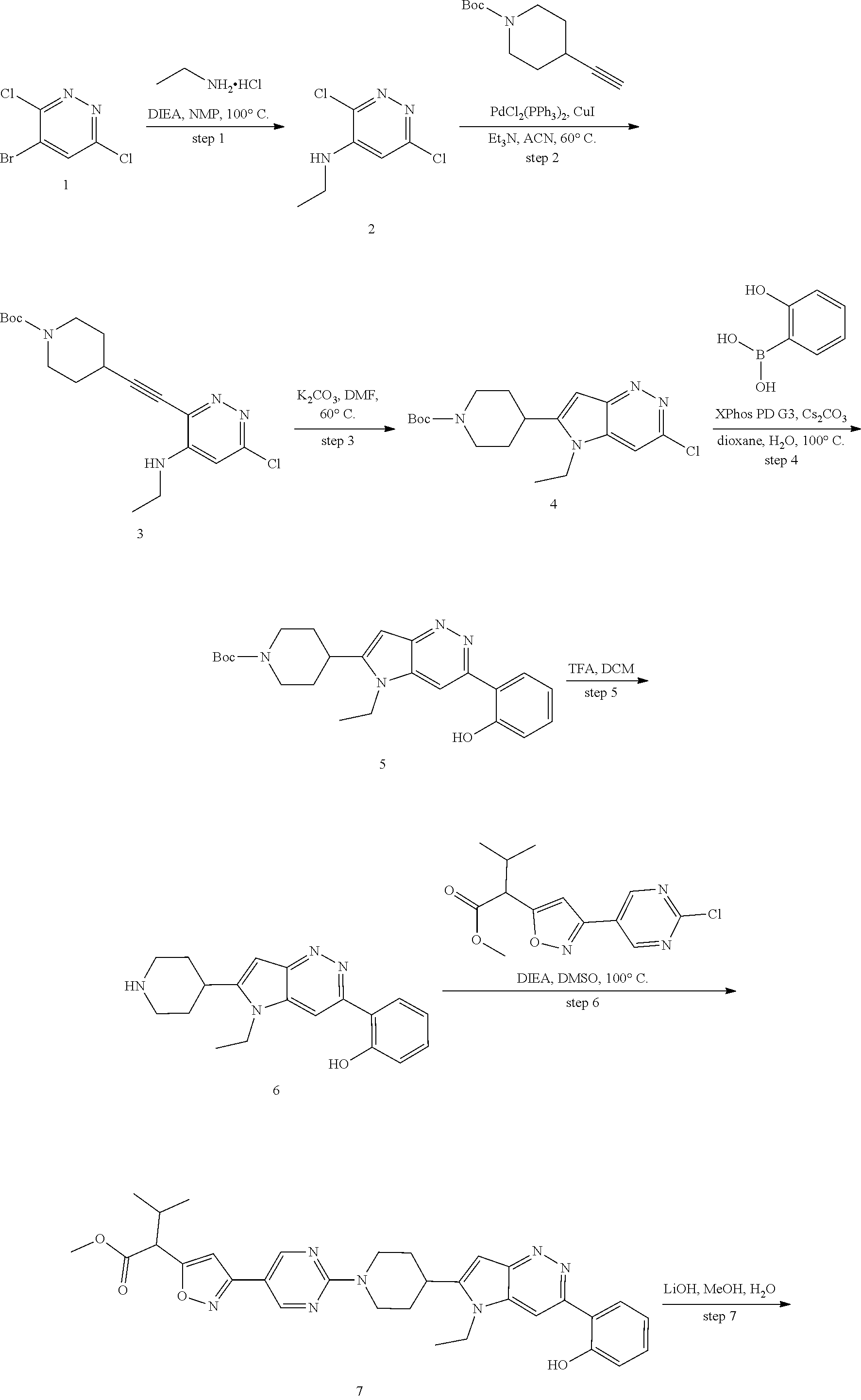

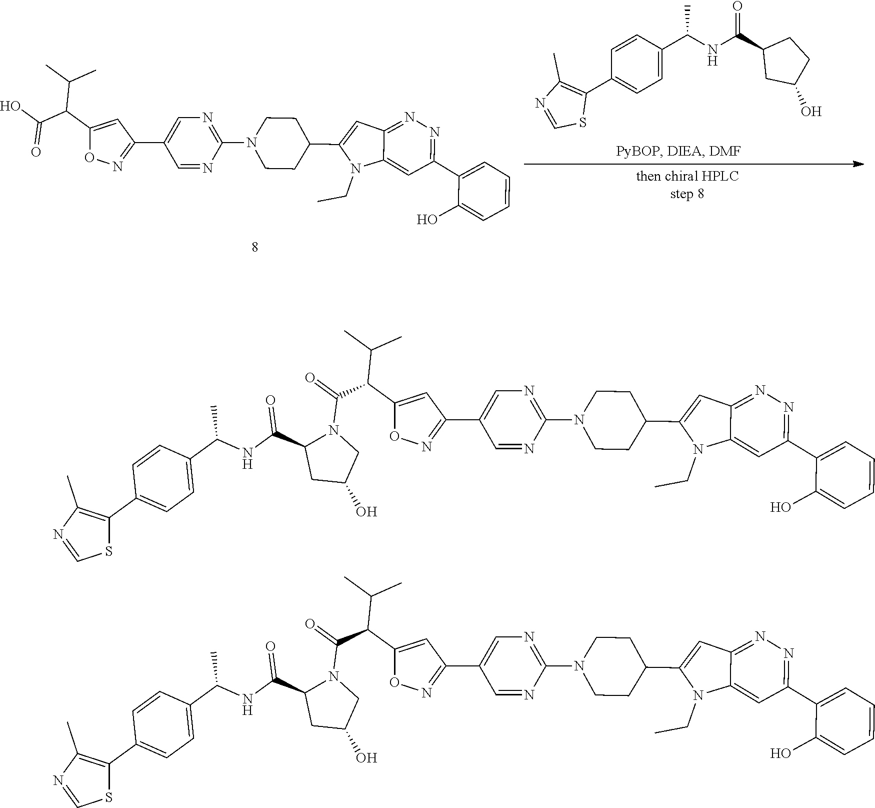 [SMILES: CCn1c(C2CCN(c3ncc(-c4cc(C(C(=O)O)C(C)C)on4)cn3)CC2)cc2nnc(-c3ccccc3O)cc21 ; Cc1ncsc1-c1ccc([C@H](C)NC(=O)[C@H]2CC[C@H](O)C2)cc1 ; CCn1c(C2CCN(c3ncc(-c4cc([C@H](C(=O)N5C[C@H](O)C[C@H]5C(=O)N[C@@H](C)c5ccc(-c6scnc6C)cc5)C(C)C)on4)cn3)CC2)cc2nnc(-c3ccccc3O)cc21 ; CCn1c(C2CCN(c3ncc(-c4cc([C@@H](C(=O)N5C[C@H](O)C[C@H]5C(=O)N[C@@H](C)c5ccc(-c6scnc6C)cc5)C(C)C)on4)cn3)CC2)cc2nnc(-c3ccccc3O)cc21]

### Step 1: Preparation of 3,6-dichloro-N-ethylpyridazin-4-amine (Intermediate 2)

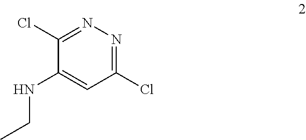

To a stirred mixture of 4-bromo-3,6-dichloropyridazine (10 g, 43.885 mmol, 1 equiv) and ethylamine hydrochloride (4.29 g, 52.662 mmol, 1.2 equiv) in NMP (100 mL) was added DIEA (17.02 g, 131.655 mmol, 3 equiv) dropwise at room temperature. After stirring for 1 h at 100° C., the mixture was allowed to cool down to room temperature. The resulting mixture was diluted with EtOAc (500 mL) then washed with water (3×300 mL). The organic layer was dried over anhydrous Na₂SO₄. After filtration, the filtrate was concentrated under reduced pressure. The crude product was used in the next step directly without further purification. LCMS (ESI) m/z: [M+H]⁺=192.

### Step 2: Preparation of tert-butyl 4-([6-chloro-4-(ethylamino)pyridazin-3-yl]ethynyl)piperidine-1-carboxylate (Intermediate 3)

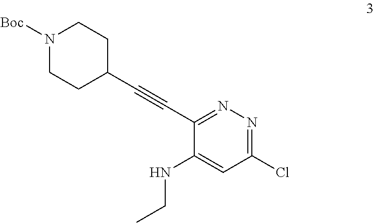 [SMILES: CCNc1cc(Cl)nnc1C#CC1CCN(C(=O)OC(C)(C)C)CC1]

To a solution of Intermediate 2 (8.7 g, 45.303 mmol, 1 equiv) and tert-butyl 4-ethynylpiperidine-1-carboxylate (11.38 g, 54.364 mmol, 1.2 equiv) in CH₃CN (100 mL) were added Et₃N (13.75 g, 135.909 mmol, 3 equiv), Pd(PPh₃)₂Cl₂ (6.36 g, 9.061 mmol, 0.2 equiv) and CuI (1.73 g, 9.061 mmol, 0.2 equiv). After stirring for 1 h at 60° C. under nitrogen atmosphere, the resulting mixture was concentrated under reduced pressure. The residue was purified by silica gel column chromatography, eluted with PE/EA (3:7) to afford Intermediate 3 (13.4 g, 72.96%) as a yellow oil. LCMS (ESI) m/z: [M+H]⁺=365.

### Step 3: Preparation of tert-butyl 4-{3-chloro-5-ethylpyrrolo[3,2-c]pyridazin-6-yl}piperidine-1-carboxylate (Intermediate 4)

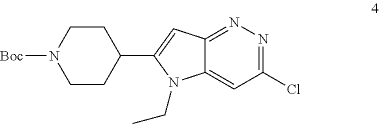 [SMILES: CCn1c(C2CCN(C(=O)OC(C)(C)C)CC2)cc2nnc(Cl)cc21]

To a stirred mixture of Intermediate 3 (14 g, 38.370 mmol, 1 equiv) in DMF (100 mL) was added K₂CO₃ (10.61 g, 76.740 mmol, 2 equiv). After stirring for 3 h at 60° C., the mixture was allowed to cool down to room temperature. The resulting mixture was diluted with EtOAc (500 mL) then washed with water (3×300 mL). The organic layer was dried over anhydrous Na₂SO₄. After filtration, the filtrate was concentrated under reduced pressure. To the residue was added EA (100 mL) and the mixture was stirred for 30 min at 0° C. The precipitated solid was collected by filtration and washed with EtOAc (3×20 mL) to afford Intermediate 4 (10 g, 67.86%) as a yellow solid. LCMS (ESI) m/z: [M+H]⁺=365.

### Step 4: Preparation of tert-butyl 4-[5-ethyl-3-(2-hydroxyphenyl)pyrrolo[3,2-c]pyridazin-6-yl]piperidine-1-carboxylate (Intermediate 5)

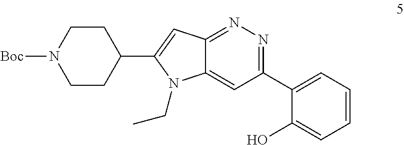 [SMILES: CCn1c(C2CCN(C(=O)OC(C)(C)C)CC2)cc2nnc(-c3ccccc3O)cc21]

To a solution of Intermediate 4 (5 g, 13.704 mmol, 1 equiv) and 2-hydroxyphenylboronic acid (5.67 g, 41.112 mmol, 3 equiv) in dioxane (50 mL) and H₂O (10 mL) were added Cs₂CO₃ (13.39 g, 41.112 mmol, 3 equiv) and XPhos Pd G3 (2.32 g, 2.741 mmol, 0.2 equiv). After stirring for 3 h at 100° C. under a nitrogen atmosphere, the mixture was allowed to cool down to room temperature. The resulting mixture was diluted with water (100 mL) and extracted with EtOAc (3×200 mL). The combined organic layers were dried over anhydrous Na₂SO₄. After filtration, the filtrate was concentrated under reduced pressure. The residue was purified by trituration with EtOAc (50 mL). This resulted in Intermediate 5 (2.9 g, 47.58%) as a yellow solid. LCMS (ESI) m/z: [M+H]⁺=423.

### Step 5: Preparation of 2-[5-ethyl-6-(piperidin-4-yl)pyrrolo[3,2-c]pyridazin-3-yl]phenol (Intermediate 6)

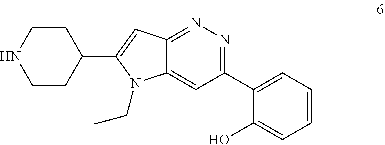 [SMILES: CCn1c(C2CCNCC2)cc2nnc(-c3ccccc3O)cc21]

To a stirred mixture of Intermediate 5 (2.9 g, 6.863 mmol, 1 equiv) in DCM (10 mL) was added TFA (3.00 mL). The resulting mixture was stirred for 1 h at room temperature. The resulting mixture was concentrated under reduced pressure. This resulted in Intermediate 6 (2.4 g, 97.61%) as a yellow oil. LCMS (ESI) m/z: [M+H]⁺=323.

### Step 6: Preparation of methyl 2-[3-(2-{4-[5-ethyl-3-(2-hydroxyphenyl)pyrrolo[3,2-c]pyridazin-6-yl]piperidin-1-yl}pyrimidin-5-yl)-1,2-oxazol-5-yl]-3-methylbutanoate (Intermediate 7)

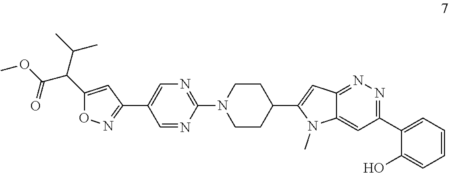 [SMILES: COC(=O)C(c1cc(-c2cnc(N3CCC(c4cc5nnc(-c6ccccc6O)cc5n4C)CC3)nc2)no1)C(C)C]

To a stirred mixture of Intermediate 6 (322 mg, 0.999 mmol, 1 equiv) and methyl 2-[3-(2-chloropyrimidin-5-yl)-1,2-oxazol-5-yl]-3-methylbutanoate (295.34 mg, 0.999 mmol, 1 equiv) in DMSO (3 mL) was added DIEA (645.41 mg, 4.995 mmol, 5 equiv) dropwise. The resulting mixture was stirred for 1 h at 100° C. The mixture was allowed to cool down to room temperature and the product was precipitated by the addition of water (10 mL). The precipitated solid was collected by filtration and washed with water (3×10 mL). This resulted in Intermediate 7 (210 mg, 34.34%) as an off-white solid. LCMS (ESI) m/z: [M+H]⁺=582.

### Step 7: Preparation of 2-[3-(2-{4-[5-ethyl-3-(2-hydroxyphenyl)pyrrolo[3,2-c]pyridazin-6-yl]piperidin-1-yl}pyrimidin-5-yl)-1,2-oxazol-5-yl]-3-methylbutanoic acid (Intermediate 8)

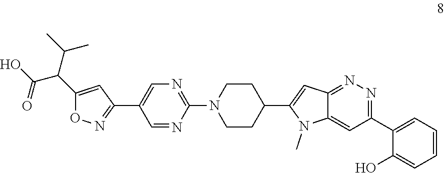 [SMILES: CC(C)C(C(=O)O)c1cc(-c2cnc(N3CCC(c4cc5nnc(-c6ccccc6O)cc5n4C)CC3)nc2)no1]

To a stirred mixture of Intermediate 7 (210 mg, 0.361 mmol, 1 equiv) in MeOH (4 mL) and H₂O (1 mL) was added LiOH (86.47 mg, 3.610 mmol, 10 equiv). After stirring for 2 h at 40° C., the mixture was allowed to cool down to room temperature. The mixture was acidified to pH 6 with 4 M HCl (aq.). The precipitated solid was collected by filtration and washed with water (3×10 mL). This resulted in Intermediate 8 (174 mg, 78.35%) as a yellow solid. LCMS (ESI) m/z: [M+H]⁺=568.

### Step 8: Preparation of (2S,4R)-1-[(2R)-2-[3-(2-{4-[5-ethyl-3-(2-hydroxyphenyl)pyrrolo[3,2-c]pyridazin-6-yl]piperidin-1-yl}pyrimidin-5-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 23) and (2S,4R)-1-[(2S)-2-[3-(2-{4-[5-ethyl-3-(2-hydroxyphenyl)pyrrolo[3,2-c]pyridazin-6-yl]piperidin-1-yl}pyrimidin-5-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 24)

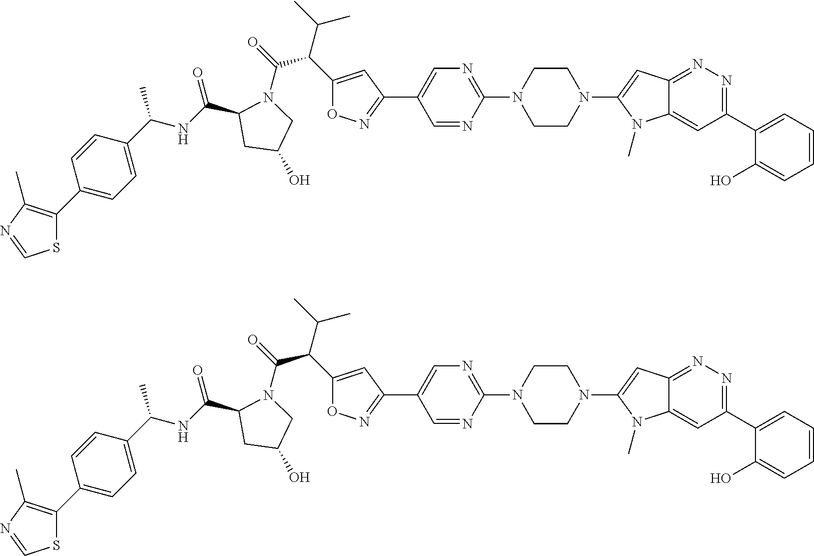 [SMILES: Cc1ncsc1-c1ccc([C@H](C)NC(=O)[C@@H]2C[C@@H](O)CN2C(=O)[C@@H](c2cc(-c3cnc(N4CCN(c5cc6nnc(-c7ccccc7O)cc6n5C)CC4)nc3)no2)C(C)C)cc1 ; Cc1ncsc1-c1ccc([C@H](C)NC(=O)[C@@H]2C[C@@H](O)CN2C(=O)[C@H](c2cc(-c3cnc(N4CCN(c5cc6nnc(-c7ccccc7O)cc6n5C)CC4)nc3)no2)C(C)C)cc1]

To a stirred mixture of Intermediate 8 (150 mg, 0.264 mmol, 1 equiv) and (2S,4R)-4-hydroxy-N-[(1S)-1-[4-(4-methyl-1,3-thiazol-5-yl)phenyl]ethyl]pyrrolidine-2-carboxamide (87.58 mg, 0.264 mmol, 1 equiv) in DMF (4 mL) were added DIEA (102.46 mg, 0.792 mmol, 3 equiv) and PyBOP (274.56 mg, 0.528 mmol, 2.0 equiv) in portions at room temperature. The resulting mixture was stirred for 2 h at room temperature. The mixture was purified by reverse phase flash chromatography with the following conditions: column, C18 silica gel; mobile phase, MeCN in water (10 mmol/L NH₄HCO₃), 0% to 100% gradient in 30 min; detector, UV 254 nm. The resulting residue (80 mg) was then purified by chiral HPLC with the following conditions: Column, CHIRALPAK ID-3, 4.6*50 mm, 3 μm; Mobile Phase A: MtBE (0.1% DIEA), Mobile Phase B: MeOH; Flow rate: 1.67 mL/min. This resulted in:

Compound 23 (36.7 mg, 15.21%) as a white solid. ¹H NMR (400 MHz, DMSO-d6) δ 14.57 (s, 1H), 8.99 (s, 1H), 8.84 (s, 2H), 8.61 (s, 1H), 8.43 (d, J=7.6 Hz, 1H), 8.23-8.16 (m, 1H), 7.45 (d, J=8.3 Hz, 2H), 7.41-7.35 (m, 2H), 7.32 (td, J=7.6, 1.6 Hz, 1H), 6.98 (t, J=7.6 Hz, 2H), 6.93 (s, 1H), 6.87-6.80 (m, 1H), 5.11 (d, J=3.6 Hz, 1H), 4.99-4.90 (m, 3H), 4.50-4.35 (m, 3H), 4.31 (s, 1H), 3.85 (d, J=9.7 Hz, 1H), 3.80-3.58 (m, 1H), 3.51 (d, J=10.7 Hz, 1H), 3.31-3.27 (m, 1H), 3.20 (t, J=12.7 Hz, 2H), 2.48-2.42 (m, 3H), 2.39-2.27 (m, 1H), 2.17-1.97 (m, 3H), 1.86-1.71 (m, 3H), 1.52-1.33 (m, 6H), 1.06-0.99 (m, 3H), 0.89-0.81 (m, 3H). LCMS (ESI) m/z: [M+H]⁺=881.35.

Compound 24 (20.4 mg, 8.53%) as a white solid. ¹H NMR (400 MHz, DMSO-d6) δ 14.57 (s, 1H), 9.02-8.94 (m, 1H), 8.86-8.78 (m, 2H), 8.61 (s, 1H), 8.28 (d, J=7.9 Hz, 1H), 8.23-8.16 (m, 1H), 7.53-7.41 (m, 1H), 7.40-7.27 (m, 4H), 6.98 (t, J=7.7 Hz, 2H), 6.93 (s, 1H), 6.86-6.81 (m, 1H), 5.14 (s, 1H), 4.96-4.82 (m, 3H), 4.49-4.41 (m, 3H), 4.30 (s, 1H), 3.95 (d, J=9.0 Hz, 1H), 3.67-3.40 (m, 2H), 3.31-3.26 (m, 1H), 3.19 (t, J=12.7 Hz, 2H), 2.47 (s, 1H), 2.41 (s, 2H), 2.40-2.32 (m, 1H), 2.14-2.02 (m, 3H), 2.00-1.71 (m, 3H), 1.52-1.29 (m, 6H), 1.02 (d, J=6.6 Hz, 3H), 0.91-0.79 (m, 3H). LCMS (ESI) m/z: [M+H]⁺=881.45.

### Preparation of (2S,4R)-1-[(2R)-2-[3-(2-{4-[5-cyclopropyl-3-(2-hydroxyphenyl)pyrrolo[3,2-c]pyridazin-6-yl]piperidin-1-yl}pyrimidin-5-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 25) and (2S,4R)-1-[(2S)-2-[3-(2-{4-[5-cyclopropyl-3-(2-hydroxyphenyl)pyrrolo[3,2-c]pyridazin-6-yl]piperidin-1-yl)pyrimidin-5-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 26)

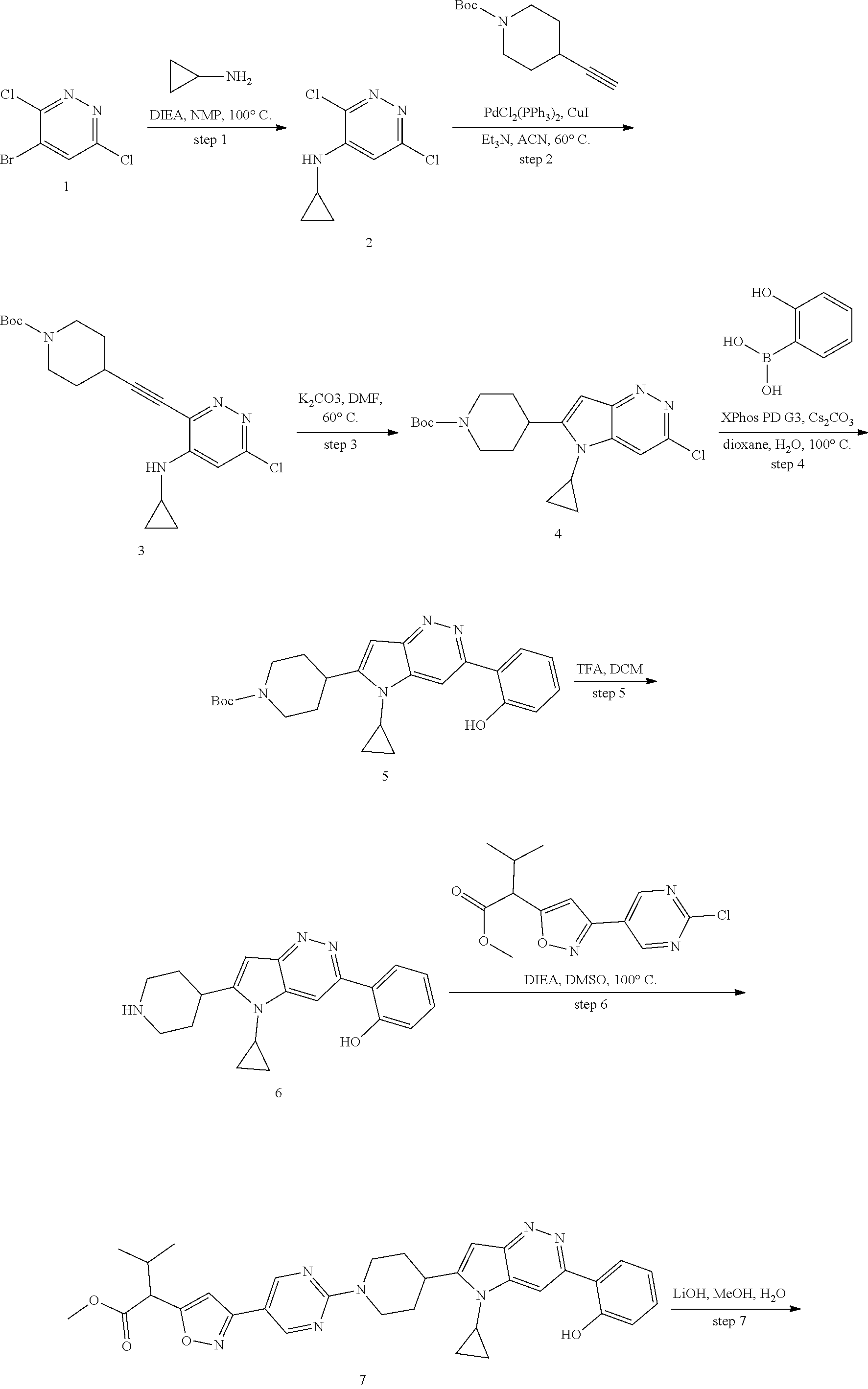

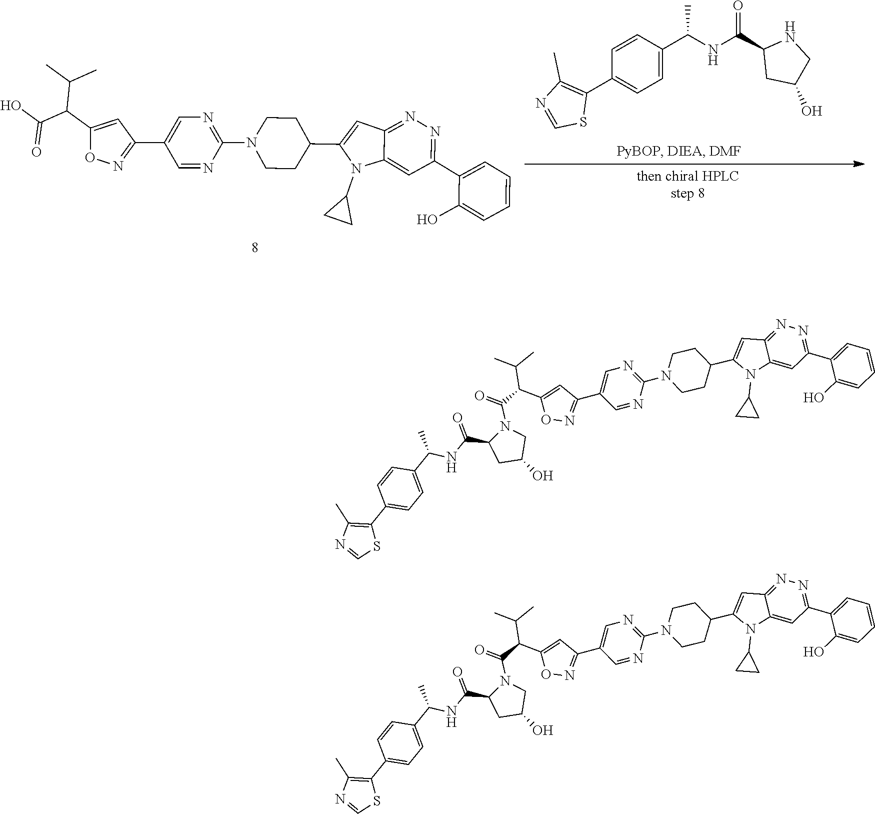 [SMILES: CC(C)C(C(=O)O)c1cc(-c2cnc(N3CCC(c4cc5nnc(-c6ccccc6O)cc5n4C4CC4)CC3)nc2)no1 ; Cc1ncsc1-c1ccc([C@H](C)NC(=O)[C@@H]2C[C@@H](O)CN2)cc1 ; Cc1ncsc1-c1ccc([C@H](C)NC(=O)[C@@H]2C[C@@H](O)CN2C(=O)[C@@H](c2cc(-c3cnc(N4CCC(c5cc6nnc(-c7ccccc7O)cc6n5C5CC5)CC4)nc3)no2)C(C)C)cc1 ; Cc1ncsc1-c1ccc([C@H](C)NC(=O)[C@@H]2C[C@@H](O)CN2C(=O)[C@H](c2cc(-c3cnc(N4CCC(c5cc6nnc(-c7ccccc7O)cc6n5C5CC5)CC4)nc3)no2)C(C)C)cc1]

### Step 1: Preparation of 3,6-dichloro-N-cyclopropylpyridazin-4-amine (Intermediate 2)

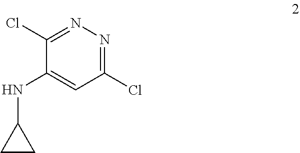 [SMILES: Clc1cc(NC2CC2)c(Cl)nn1]

To a stirred mixture of 4-bromo-3,6-dichloropyridazine (6 g, 26.331 mmol, 1 equiv) and DIEA (10.21 g, 78.993 mmol, 3 equiv) in NMP (50 mL) was added aminocyclopropane (2.26 g, 39.496 mmol, 1.5 equiv) dropwise at 0° C. under nitrogen atmosphere. The resulting mixture was stirred for 2 h at 100° C. After cooling down to room temperature, the resulting mixture was extracted with EtOAc (2×100 mL). The combined organic layers were washed with brine (2×200 mL) then dried over anhydrous Na₂SO₄. After filtration, the filtrate was concentrated under reduced pressure. The residue was purified by silica gel column chromatography, eluted with PE/EA (5:1) to afford Intermediate 2 (3.4 g, 63.28%) as an off-white solid. LCMS (ESI) m/z: [M+H]⁺=204.

### Step 2: Preparation of tert-butyl 4-([6-chloro-4-(cyclopropylamino)pyridazin-3-yl]ethynyl)piperidine-1-carboxylate (Intermediate 3)

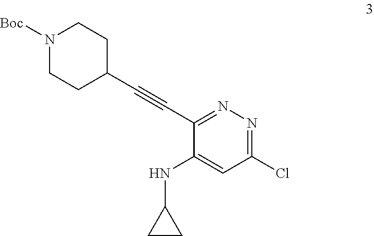 [SMILES: CC(C)(C)OC(=O)N1CCC(C#Cc2nnc(Cl)cc2NC2CC2)CC1]

To a stirred mixture of intermediate 2 (1.7 g, 8.331 mmol, 1 equiv) and tert-butyl 4-ethynylpiperidine-1-carboxylate (2.62 g, 12.496 mmol, 1.5 equiv) in CH₃CN (85 mL) were added Pd(PPh₃)₂Cl₂ (1.17 g, 1.666 mmol, 0.2 equiv), CuI (0.32 g, 1.666 mmol, 0.2 equiv) and Et₃N (2.53 g, 24.993 mmol, 3 equiv) at room temperature under nitrogen atmosphere. The resulting mixture was stirred for 3 h at 60° C. The resulting mixture was concentrated under reduced pressure. The residue was purified by silica gel column chromatography, eluted with PE/EA (2:1) to afford Intermediate 3 (506 mg, 16.11%) as a yellow solid. LCMS (ESI) m/z: [M+H]⁺=377.

### Step 3: Preparation of tert-butyl 4-{3-chloro-5-cyclopropylpyrrolo[3,2-c]pyridazin-6-yl}piperidine-1-carboxylate (Intermediate 4)

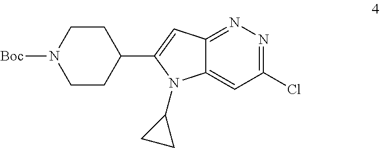 [SMILES: CC(C)(C)OC(=O)N1CCC(c2cc3nnc(Cl)cc3n2C2CC2)CC1]

A mixture of Intermediate 3 (500 mg, 1.327 mmol, 1 equiv) and K₂CO₃ (733.40 mg, 5.308 mmol, 4 equiv) in DMF (10 mL) was stirred for 3 h at 60° C. under nitrogen atmosphere. After cooling down to room temperature, the mixture was dissolved in EtOAc (100 mL). The resulting mixture was washed with brine (2×100 mL) then dried over anhydrous Na₂SO₄. After filtration, the filtrate was concentrated under reduced pressure. The residue was purified by silica gel column chromatography, eluted with PE/EA (2:1) to afford Intermediate 4 (413 mg, 80.95%) as a red solid. LCMS (ESI) m/z: [M+H]⁺=377.

### Step 4: Preparation of tert-butyl 4-[5-cyclopropyl-3-(2-hydroxyphenyl)pyrrolo[3,2-c]pyridazin-6-yl]piperidine-1-carboxylate (Intermediate 5)

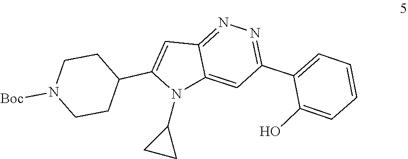 [SMILES: CC(C)(C)OC(=O)N1CCC(c2cc3nnc(-c4ccccc4O)cc3n2C2CC2)CC1]

To a solution of intermediate 4 (413 mg, 1.096 mmol, 1 equiv) and 2-hydroxyphenylboronic acid (755.73 mg, 5.480 mmol, 5 equiv) in dioxane (10 mL) and H₂O (2 mL) were added XPhos Pd G3 (139.13 mg, 0.164 mmol, 0.15 equiv) and Cs₂CO₃ (1071.11 mg, 3.288 mmol, 3 equiv) in portions at room temperature under nitrogen atmosphere. The resulting mixture was stirred for 1.5 h at 100° C. The mixture was purified by reverse phase flash chromatography with the following conditions: column, C18 silica gel; mobile phase, MeCN in water (10 mmol/L NH₄HCO₃), 5% to 95% gradient in 35 min; detector, UV 254 nm. This resulted in Intermediate 5 (296 mg, 60.30%) as a yellow solid. LCMS (ESI) m/z: [M+H]⁺=435.

### Step 5: Preparation of 2-[5-cyclopropyl-6-(piperidin-4-yl)pyrrolo[3,2-c]pyridazin-3-yl]phenol (Intermediate 6)

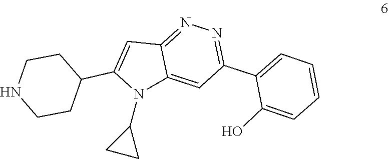 [SMILES: Oc1ccccc1-c1cc2c(cc(C3CCNCC3)n2C2CC2)nn1]

A mixture of Intermediate 5 (296 mg, 0.681 mmol, 1 equiv) in TFA (1 mL) and DCM (3 mL) was stirred for 1 h at room temperature under nitrogen atmosphere. The resulting mixture was concentrated under reduced pressure. This resulted in Intermediate 6 (186 mg, 81.65%) as a yellow solid. LCMS (ESI) m/z: [M+H]⁺=335.

### Step 6: Preparation of methyl 2-[3-(2-{4-[5-cyclopropyl-3-(2-hydroxyphenyl)pyrrolo[3,2-c]pyridazin-6-yl]piperidin-1-yl}pyrimidin-5-yl)-1,2-oxazol-5-yl]-3-methylbutanoate (Intermediate 7)

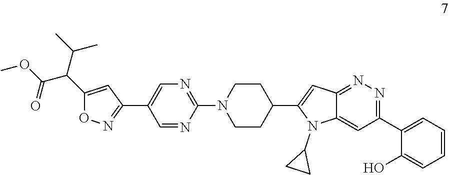 [SMILES: COC(=O)C(c1cc(-c2cnc(N3CCC(c4cc5nnc(-c6ccccc6O)cc5n4C4CC4)CC3)nc2)no1)C(C)C]

To a stirred mixture of Intermediate 6 (186 mg, 0.556 mmol, 1 equiv) and methyl 2-[3-(2-chloropyrimidin-5-yl)-1,2-oxazol-5-yl]-3-methylbutanoate (164.47 mg, 0.556 mmol, 1 equiv) in DMSO (3 mL) was added DIEA (581.28 μL, 3.336 mmol, 6 equiv) dropwise at 100° C. under nitrogen atmosphere. The reaction mixture was stirred for 1 h at this temperature. After cooling down to room temperature, the product was precipitated by the addition of water (20 mL). The precipitated solid was collected by filtration. This resulted in Intermediate 7 (175 mg, 46.64%) as a brown solid. LCMS (ESI) m/z: [M+H]⁺=594.

### Step 7: Preparation of 2-[3-(2-{4-[5-cyclopropyl-3-(2-hydroxyphenyl)pyrrolo[3,2-c]pyridazin-6-yl]piperidin-1-yl}pyrimidin-5-yl)-1,2-oxazol-5-yl]-3-methylbutanoic acid (Intermediate 8)

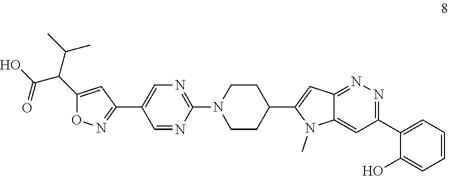 [SMILES: CC(C)C(C(=O)O)c1cc(-c2cnc(N3CCC(c4cc5nnc(-c6ccccc6O)cc5n4C)CC3)nc2)no1]

A mixture of intermediate 7 (175 mg, 0.295 mmol, 1 equiv) and LiOH·H₂O (123.68 mg, 2.950 mmol, 10 equiv) in MeOH (4 mL) and H₂O (1 mL) was stirred for 1 h at 60° C. under nitrogen atmosphere. After cooling down to room temperature, the mixture was acidified to pH 5 with HCl (aq.). The precipitated solid was collected by filtration and washed with EtOAc (2×50 mL). This resulted in Intermediate 8 (126 mg, 73.74%) as an off-white solid. LCMS (ESI) m/z: [M+H]⁺=580.

### Step 8: Preparation of (2S,4R)-1-[(2R)-2-[3-(2-{4-[5-cyclopropyl-3-(2-hydroxyphenyl)pyrrolo[3,2-c]pyridazin-6-yl]piperidin-1-yl}pyrimidin-5-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 25) and (2S,4R)-1-[(2S)-2-[3-(2-{4-[5-cyclopropyl-3-(2-hydroxyphenyl)pyrrolo[3,2-c]pyridazin-6-yl]piperidin-1-yl}pyrimidin-5-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 26)

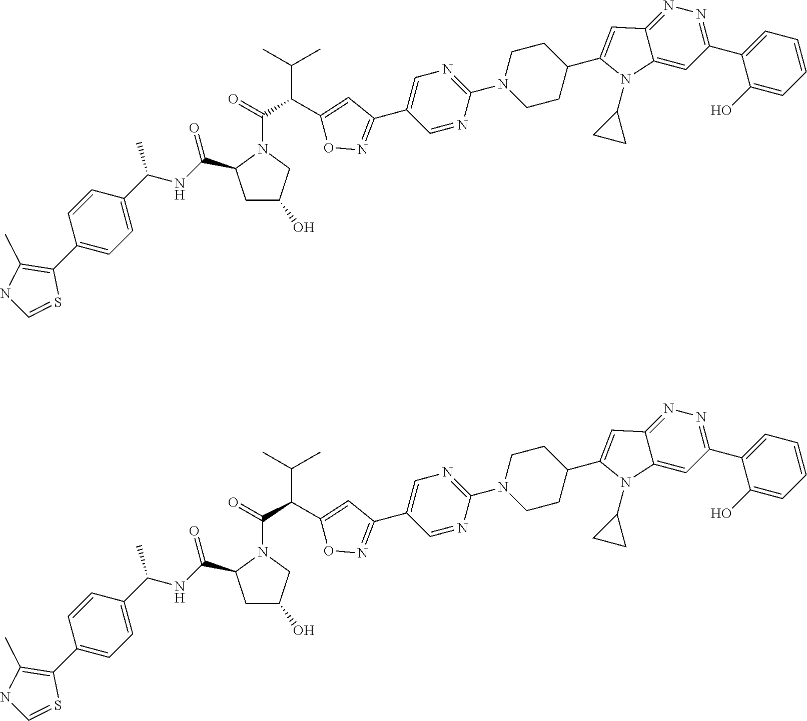

To a stirred mixture of Intermediate 8 (126 mg, 0.217 mmol, 1 equiv) and (2S,4R)-4-hydroxy-N-[(1 S)-1-[4-(4-methyl-1,3-thiazol-5-yl)phenyl]ethyl]pyrrolidine-2-carboxamide (72.04 mg, 0.217 mmol, 1 equiv) in DMF (2.5 mL) were added PyBOP (226.24 mg, 0.434 mmol, 2 equiv) and DIEA (84.28 mg, 0.651 mmol, 3 equiv) at room temperature under nitrogen atmosphere. The resulting mixture was stirred for 1 h at room temperature. The mixture was purified by Prep-HPLC with the following conditions: Column, XBridge Shield RP18 OBD, 30*150 mm, 5 μm; Mobile phase, water (10 mmol/L NH₄HCO₃+0.1% NH₃·H₂O) and CH₃CN (hold 2% CH₃CN in 2 min, up to 46% in 1 min then up to 68% in 6.5 min); Detector, UV 254 nm. The resulting residue (82 mg) was then purified by chiral HPLC with the following conditions: Column, CHIRALPAK ID, 2*25 cm, 5 μm; Mobile phase, MtBE (10 mM NH₃-MeOH) and MeOH (hold 50% MeOH for 30 min); Detector, UV 254 nm. This resulted in:

Compound 25 (52 mg, 40.69%) as a white solid. ¹H NMR (400 MHz, DMSO-d6) δ 14.31 (s, 1H), 8.99 (s, 1H), 8.85 (d, J=2.9 Hz, 2H), 8.44 (d, J=7.7 Hz, 1H), 8.35 (s, 1H), 8.14 (d, J=7.9 Hz, 1H), 7.45 (d, J=8.2 Hz, 2H), 7.38 (d, J=8.3 Hz, 2H), 7.32 (td, J=7.6, 1.5 Hz, 1H), 6.99 (t, J=8.2 Hz, 2H), 6.93 (s, 1H), 6.82 (d, J=3.3 Hz, 1H), 5.12 (d, J=3.6 Hz, 1H), 5.02-4.87 (m, 3H), 4.39 (t, J=7.9 Hz, 1H), 4.31 (s, 1H), 3.85 (d, J=9.7 Hz, 1H), 3.76 (dd, J=10.3, 4.2 Hz, 1H), 3.59-3.42 (m, 3H), 3.18 (t, J=12.7 Hz, 2H), 2.45 (d, J=6.6 Hz, 3H), 2.40-2.29 (m, 1H), 2.21 (d, J=12.7 Hz, 2H), 2.11-1.87 (m, 1H), 1.85-1.64 (m, 3H), 1.49 (d, J=6.9 Hz, 5H), 1.17 (d, J=3.6 Hz, 2H), 1.02 (t, J=5.4 Hz, 3H), 0.85 (t, J=7.4 Hz, 3H). LCMS (ESI) m/z: [M+H]⁺=893.30.

Compound 26 (35.6 mg, 29.10%) as a white solid. ¹H NMR (400 MHz, DMSO-d6) δ 14.31 (d, J=4.1 Hz, 1H), 8.98 (d, J=15.0 Hz, 1H), 8.83 (d, J=19.3 Hz, 2H), 8.35 (s, 1H), 8.29 (d, J=7.8 Hz, 1H), 8.14 (dd, J=8.1, 1.6 Hz, 1H), 7.53-7.42 (m, 2H), 7.40-7.26 (m, 4H), 7.04-6.91 (m, 3H), 6.82 (d, J=3.2 Hz, 1H), 5.15 (dd, J=3.6, 1.4 Hz, 1H), 5.08-4.83 (m, 3H), 4.51 (dt, J=51.6, 7.4 Hz, 1H), 4.29 (d, J=7.5 Hz, 1H), 3.95 (d, J=9.0 Hz, 1H), 3.67-3.41 (m, 4H), 3.17 (t, J=12.7 Hz, 2H), 2.44 (d, J=24.0 Hz, 3H), 2.39-2.31 (m, 1H), 2.21 (d, J=12.7 Hz, 2H), 2.12-1.90 (m, 1H), 1.75 (ddt, J=35.8, 14.3, 8.3 Hz, 3H), 1.49 (d, J=6.9 Hz, 1H), 1.35 (dd, J=6.9, 3.5 Hz, 4H), 1.18 (q, J=3.7 Hz, 2H), 1.02 (d, J=6.5 Hz, 2H), 0.92-0.77 (m, 4H). LCMS (ESI) m/z: [M+H]⁺=893.30.

### Preparation of (2S,4R)-4-hydroxy-1-[(2R)-2-[3-(2-{4-[3-(2-hydroxyphenyl)-5-methylpyrrolo[3,2-c]pyridazin-6-yl]piperazin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 27)

### Preparation of 2-(5-methyl-6-(piperazin-1-yl)-5H-pyrrolo[3,2-c]pyridazin-3-yl)phenol (Intermediate 8)

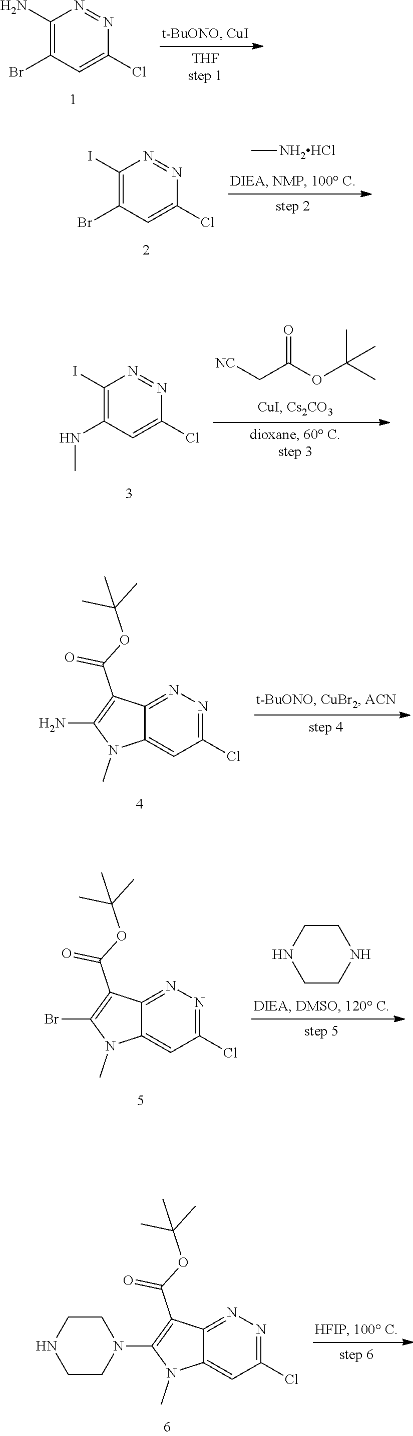

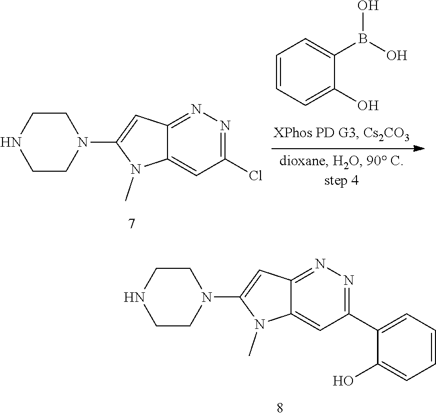 [SMILES: OB(O)c1ccccc1O ; Cn1c(N2CCNCC2)cc2nnc(Cl)cc21 ; Cn1c(N2CCNCC2)cc2nnc(-c3ccccc3O)cc21]

### Step 1: Preparation of 4-bromo-6-chloro-3-iodopyridazine (Intermediate 2)

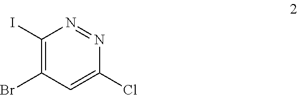

To a stirred solution of 4-bromo-6-chloropyridazin-3-amine (10 g, 47.975 mmol, 1 equiv) and CuI (18.27 g, 95.950 mmol, 2.0 equiv) in THF (50 mL) was added t-BuONO (9.89 g, 95.950 mmol, 2.0 equiv) at room temperature under nitrogen atmosphere. The resulting mixture was stirred for 12 h at 60° C. The mixture was concentrated under reduced pressure and then was purified by silica gel column chromatography, eluted with PE/EA (1:1) to afford intermediate 2 (6.0 g, 39.17%) as a yellow solid. LCMS (ESI) m/z [M+H]⁺=319.

### Step 2: Preparation of 6-chloro-3-iodo-N-methylpyridazin-4-amine (Intermediate 3)

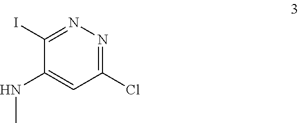

A mixture of Intermediate 2 (2.00 g, 6.263 mmol, 1.00 equiv), methylamine hydrochloride (0.63 g, 9.395 mmol, 1.50 equiv) and DIEA (2.43 g, 18.789 mmol, 3.00 equiv) in NMP (10 mL) was stirred for 3 h at 100° C. After cooling down to room temperature, the mixture was purified by reverse phase flash chromatography with the following conditions: column, C18 silica gel; mobile phase, MeCN in water (0.1% FA), 10% to 100% gradient in 40 min; detector, UV 254 nm. This resulted in intermediate 3 (1.1 g, 65.17%) as a brown solid. LCMS (ESI) m/z [M+H]⁺=270.

### Step 3: Preparation of tert-butyl 6-amino-3-chloro-5-methyl-5H-pyrrolo[3,2-c]pyridazine-7-carboxylate (Intermediate 4)

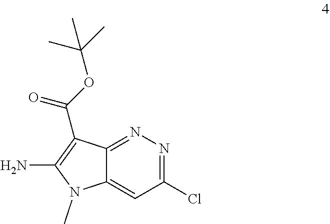 [SMILES: Cn1c(N)c(C(=O)OC(C)(C)C)c2nnc(Cl)cc21]

A mixture of intermediate 3 (1.00 g, 3.711 mmol, 1.00 equiv), tert-butyl 2-cyanoacetate (1.57 g, 11.133 mmol, 3.00 equiv), CuI (0.14 g, 0.742 mmol, 0.20 equiv) and Cs₂CO₃ (3.63 g, 11.133 mmol, 3.00 equiv) in dioxane (15 mL) was stirred for 4 h at 60° C. under argon atmosphere. After cooling down to room temperature, the resulting mixture was diluted with EtOAc (100 mL), washed with brine (3×100 mL) then dried over anhydrous Na₂SO₄. After filtration, the filtrate was concentrated under reduced pressure to afford intermediate 4 (980 mg, 93.40%) as a brown solid. LCMS (ESI) m/z [M+H]⁺=283.

### Step 4: Preparation of tert-butyl 6-bromo-3-chloro-5-methyl-5H-pyrrolo[3,2-c]pyridazine-7-carboxylate (Intermediate 5)

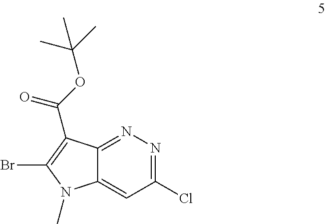 [SMILES: Cn1c(Br)c(C(=O)OC(C)(C)C)c2nnc(Cl)cc21]

A mixture of intermediate 4 (900.0 mg, 3.183 mmol, 1.00 equiv), t-BuONO (656.5 mg, 6.366 mmol, 2.00 equiv) and CuBr₂ (1066.4 mg, 4.774 mmol, 1.50 equiv) in ACN (10 mL) was stirred for 3 h at room temperature. The mixture was purified by reverse phase flash chromatography with the following conditions: column, C18 silica gel; mobile phase, MeCN in water (0.1% FA), 10% to 100% gradient in 40 min; detector, UV 254 nm. This resulted in intermediate 5 (220 mg, 18.94%) as a brown solid. LCMS (ESI) m/z [M+H]⁺=346.

### Step 5: Preparation of tert-butyl 3-chloro-5-methyl-6-(piperazin-1-yl)pyrrolo[3,2-c]pyridazine-7-carboxylate (Intermediate 6)

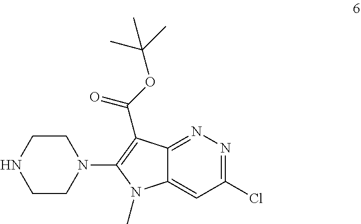 [SMILES: Cn1c(N2CCNCC2)c(C(=O)OC(C)(C)C)c2nnc(Cl)cc21]

A solution of intermediate 5 (220 mg, 0.635 mmol, 1 equiv), DIEA (246.11 mg, 1.905 mmol, 3.0 equiv) and piperazine (218.69 mg, 2.540 mmol, 4 equiv) in DMSO (4 mL) was stirred for 2 h at 120° C. under nitrogen atmosphere. After cooling down to room temperature, the mixture was purified by reverse phase flash chromatography with the following conditions: column, C18 silica gel; mobile phase, MeCN in water (10 mmol/L NH₄HCO₃), 0% to 50% gradient in 40 min; detector, UV 254 nm. This resulted in Intermediate 6 (200 mg, 89.56%) as a yellow oil. LCMS (ESI) m/z [M+H]⁺=352.

### Step 6: Preparation of 1-{3-chloro-5-methylpyrrolo[3,2-c]pyridazin-6-yl}piperazine (Intermediate 7)

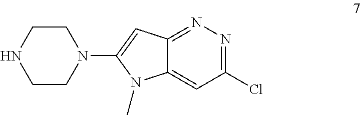 [SMILES: Cn1c(N2CCNCC2)cc2nnc(Cl)cc21]

A solution of Intermediate 6 (200 mg, 0.568 mmol, 1 equiv) in HFIP (4 mL) was stirred for 12 h at 100° C. under nitrogen atmosphere. The resulting mixture was concentrated under reduced pressure. This resulted in intermediate 7 (150 mg, 104.83%) as a yellow oil which was used in the next step directly without further purification. LCMS (ESI) m/z [M+H]⁺=252.

### Step 7: Preparation of 2-[5-methyl-6-(piperazin-1-yl)pyrrolo[3,2-c]pyridazin-3-yl]phenol (Intermediate 8)

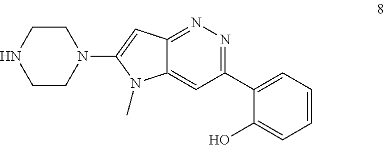 [SMILES: Cn1c(N2CCNCC2)cc2nnc(-c3ccccc3O)cc21]

To a solution of Intermediate 7 (150 mg, 0.596 mmol, 1 equiv) and 2-hydroxyphenylboronic acid (246.58 mg, 1.788 mmol, 3.0 equiv) in dioxane (5 mL) and H₂O (1 mL) were added Cs₂CO₃ (582.47 mg, 1.788 mmol, 3.0 equiv) and XPhos Pd G3 (100.88 mg, 0.119 mmol, 0.2 equiv). After stirring for 2 h at 90° C. under a nitrogen atmosphere, the mixture was cooled down to room temperature. The mixture was purified by reverse phase flash chromatography with the following conditions: column, C18 silica gel; mobile phase, MeCN in water (0.1% FA), 0% to 100% gradient in 50 min; detector, UV 254 nm. This resulted in Intermediate 8 (75 mg, 40.68%) as a yellow solid. LCMS (ESI) m/z [M+H]⁺=310.

### Preparation of (2S,4R)-4-hydroxy-1-[(2R)-2-[3-(2-{4-[3-(2-hydroxyphenyl)-5-methylpyrrolo[3,2-c]pyridazin-6-yl]piperazin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 27)

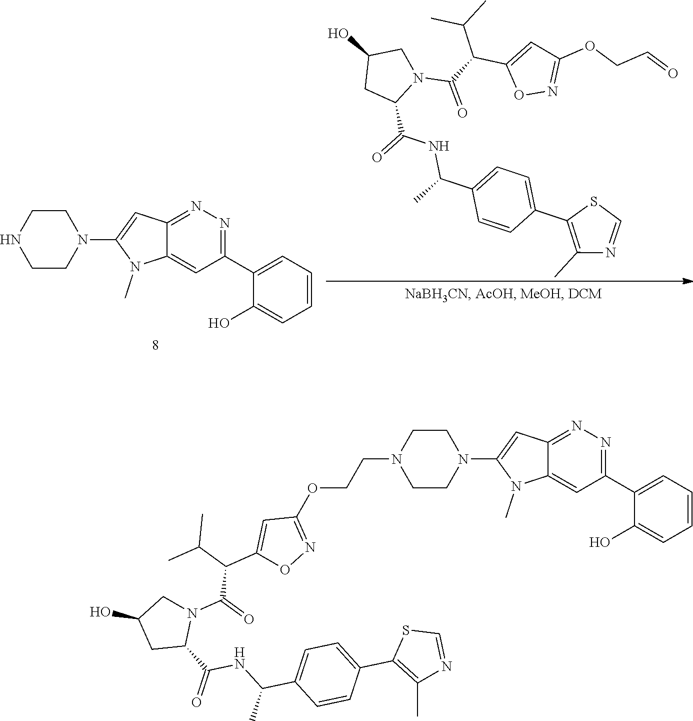 [SMILES: Cn1c(N2CCNCC2)cc2nnc(-c3ccccc3O)cc21 ; Cc1ncsc1-c1ccc([C@H](C)NC(=O)[C@@H]2C[C@@H](O)CN2C(=O)[C@@H](c2cc(OCC=O)no2)C(C)C)cc1 ; Cc1ncsc1-c1ccc([C@H](C)NC(=O)[C@@H]2C[C@@H](O)CN2C(=O)[C@@H](c2cc(OCCN3CCN(c4cc5nnc(-c6ccccc6O)cc5n4C)CC3)no2)C(C)C)cc1]

A mixture of intermediate 8 (15 mg, 0.048 mmol, 1 equiv) and (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (26.21 mg, 0.048 mmol, 1.0 equiv) in MeOH (1 mL) and DCM (1 mL) was stirred for 1 h at room temperature. To the above mixture was added NaBH₃CN (15.23 mg, 0.240 mmol, 5.0 equiv) and AcOH (cat.) at room temperature. The resulting mixture was stirred for 1 h at room temperature. The mixture was purified by Prep-HPLC with the following conditions: Column, XBridge Shield RP18 OBD, 19*150 mm, 5 μm; Mobile Phase A: water (10 mmol/L NH₄HCO₃), Mobile Phase B: CH₃CN; Flow rate: 25 mL/min; Gradient: 42% B to 55% B in 7 min, then 55% B; Detector, UV 254/220 nm. This resulted in Compound 27 (16.4 mg, 40.03%) as a yellow solid. ¹H NMR (400 MHz, Methanol-d4) δ 8.87 (s, 1H), 8.17 (d, J=12.3 Hz, 1H), 8.04-7.96 (m, 1H), 7.40-7.24 (m, 5H), 7.01-6.93 (m, 2H), 6.21-6.15 (m, 1H), 6.04 (s, 1H), 5.03 (d, J=7.0 Hz, 1H), 4.51 (t, J=8.2 Hz, 1H), 4.46-4.37 (m, 3H), 3.84 (dd, J=10.9, 4.1 Hz, 1H), 3.77-3.65 (m, 3H), 3.65-3.52 (m, 2H), 3.52-3.47 (m, 1H), 3.34 (s, 3H), 2.92 (t, J=5.5 Hz, 2H), 2.84 (d, J=5.0 Hz, 4H), 2.46-2.32 (m, 4H), 2.22-2.13 (m, 1H), 2.00-1.87 (m, 1H), 1.59-1.52 (m, 3H), 1.06 (d, J=6.5 Hz, 3H), 0.95-0.87 (m, 3H). LCMS (ESI) m/z: [M+H]⁺=834.3.

### Preparation of (2S,4R)-4-hydroxy-1-[(2R)-2-[3-(2-{4-[3-(2-hydroxyphenyl)-5-methylpyrrolo[3,2-c]pyridazin-6-yl]piperazin-1-yl}pyrimidin-5-yl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 28) and (2S,4R)-4-hydroxy-1-[(2S)-2-[3-(2-{4-[3-(2-hydroxyphenyl)-5-methylpyrrolo[3,2-c]pyridazin-6-yl]piperazin-1-yl}pyrimidin-5-yl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 29)

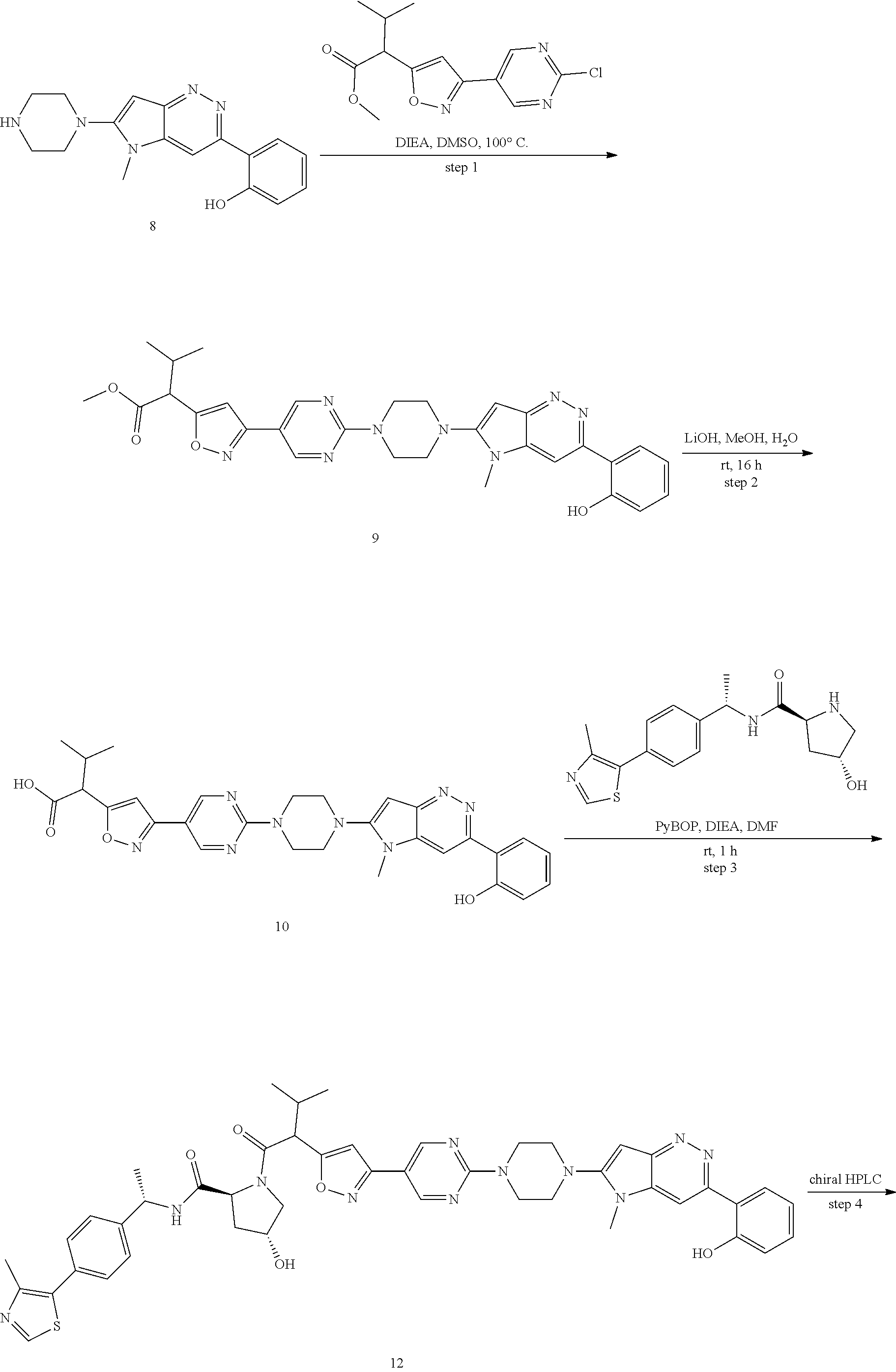 [SMILES: Cn1c(N2CCNCC2)cc2nnc(-c3ccccc3O)cc21 ; COC(=O)C(c1cc(-c2cnc(Cl)nc2)no1)C(C)C ; COC(=O)C(c1cc(-c2cnc(N3CCN(c4cc5nnc(-c6ccccc6O)cc5n4C)CC3)nc2)no1)C(C)C ; CC(C)C(C(=O)O)c1cc(-c2cnc(N3CCN(c4cc5nnc(-c6ccccc6O)cc5n4C)CC3)nc2)no1 ; Cc1ncsc1-c1ccc([C@H](C)NC(=O)[C@@H]2C[C@@H](O)CN2)cc1 ; Cc1ncsc1-c1ccc([C@H](C)NC(=O)[C@@H]2C[C@@H](O)CN2C(=O)C(c2cc(-c3cnc(N4CCN(c5cc6nnc(-c7ccccc7O)cc6n5C)CC4)nc3)no2)C(C)C)cc1]

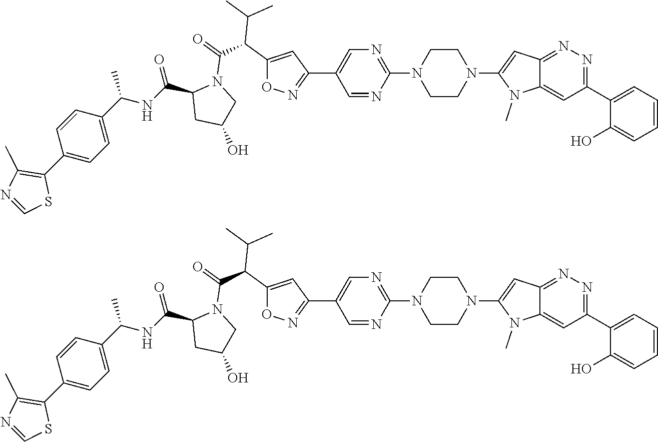 [SMILES: Cc1ncsc1-c1ccc([C@H](C)NC(=O)[C@@H]2C[C@@H](O)CN2C(=O)[C@@H](c2cc(-c3cnc(N4CCN(c5cc6nnc(-c7ccccc7O)cc6n5C)CC4)nc3)no2)C(C)C)cc1 ; Cc1ncsc1-c1ccc([C@H](C)NC(=O)[C@@H]2C[C@@H](O)CN2C(=O)[C@H](c2cc(-c3cnc(N4CCN(c5cc6nnc(-c7ccccc7O)cc6n5C)CC4)nc3)no2)C(C)C)cc1]

### Step 1: Preparation of methyl 2-[3-(2-{4-[3-(2-hydroxyphenyl)-5-methylpyrrolo[3,2-c]pyridazin-6-yl]piperazin-1-yl}pyrimidin-5-yl)-1,2-oxazol-5-yl]-3-methylbutanoate (Intermediate 9)

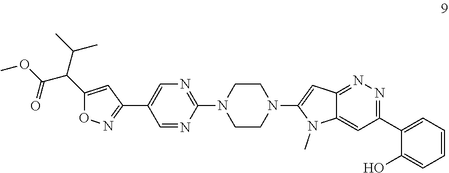 [SMILES: COC(=O)C(c1cc(-c2cnc(N3CCN(c4cc5nnc(-c6ccccc6O)cc5n4C)CC3)nc2)no1)C(C)C]

A solution of Intermediate 8 (75 mg, 0.242 mmol, 1 equiv), methyl 2-[3-(2-chloropyrimidin-5-yl)-1,2-oxazol-5-yl]-3-methylbutanoate (86.03 mg, 0.290 mmol, 1.2 equiv) and DIEA (94.00 mg, 0.726 mmol, 3.0 equiv) in DMSO (3 ml-) was stirred for 2 h at 100° C. under nitrogen atmosphere. After cooling down to room temperature, the mixture was purified by reverse phase flash chromatography with the following conditions: column, C18 silica gel; mobile phase, MeCN in water (0.1% FA), 0% to 50% gradient in 40 min; detector, UV 254 nm. This resulted in Intermediate 9 (70 mg, 50.78%) as a yellow solid. LCMS (ESI) m/z [M+H]⁺=569.

### Step 2: Preparation of 2-[3-(2-{4-[3-(2-hydroxyphenyl)-5-methylpyrrolo[3,2-c]pyridazin-6-yl]piperazin-1-yl}pyrimidin-5-yl)-1,2-oxazol-5-yl]-3-methylbutanoic acid (Intermediate 10)

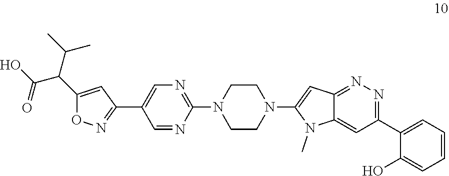 [SMILES: CC(C)C(C(=O)O)c1cc(-c2cnc(N3CCN(c4cc5nnc(-c6ccccc6O)cc5n4C)CC3)nc2)no1]

To a stirred solution of Intermediate 9 (80.00 mg, 0.140 mmol, 1.00 equiv) in MeOH (5.00 mL) and H₂O (5.00 mL) was added LiOH·H₂O (29.40 mg, 0.700 mmol, 5.00 equiv) at room temperature. The resulting mixture was stirred for 16 h at room temperature. The mixture was acidified to pH 3 with HCl (1 M). The resulting mixture was concentrated under reduced pressure. The residue was purified by reverse phase flash chromatography with the following conditions: column, C18 silica gel; mobile phase, ACN in H₂O (0.1% FA), 0% to 100% gradient in 30 min; detector, UV 254/220 nm. This resulted in Intermediate 10 (78 mg, 99.11%) as a yellow solid. LCMS (ESI) m/z: [M+H]⁺=555.

### Step 3: Preparation of (2S,4R)-4-hydroxy-1-[2-[3-(2-{4-[3-(2-hydroxyphenyl)-5-methylpyrrolo[3,2-c]pyridazin-6-yl]piperazin-1-yl}pyrimidin-5-yl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Intermediate 12)

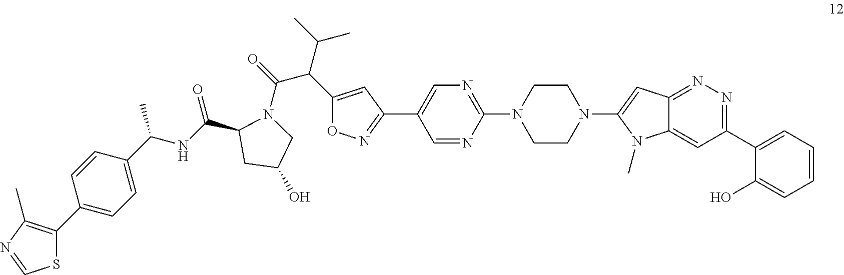 [SMILES: Cc1ncsc1-c1ccc([C@H](C)NC(=O)[C@@H]2C[C@@H](O)CN2C(=O)C(c2cc(-c3cnc(N4CCN(c5cc6nnc(-c7ccccc7O)cc6n5C)CC4)nc3)no2)C(C)C)cc1]

To a stirred solution of Intermediate 10 (78.00 mg, 0.140 mmol, 1.00 equiv) in DMF (5.00 mL) were added PyBOP (218.40 mg, 0.420 mmol, 3.00 equiv) and DIEA (90.30 mg, 0.700 mmol, 5.00 equiv) at room temperature. To the above mixture was added (2S,4R)-4-hydroxy-N-[(1S)-1-[4-(4-methyl-1,3-thiazol-5-yl)phenyl]ethyl]pyrrolidine-2-carboxamide (46.48 mg, 0.140 mmol, 1.00 equiv) at room temperature. The resulting mixture was stirred for 1 h at room temperature. The mixture was purified by reverse phase flash chromatography with the following conditions: column, C18 silica gel; mobile phase, ACN in H₂O (10 mmol/L NH₄HCO₃), 0% to 100% gradient in 30 min; detector, UV 254/220 nm. This resulted in Intermediate 12 (120 mg, 98.36%) as a white solid. LCMS (ESI) m/z: [M+H]⁺=868.

### Step 4: Preparation of (2S,4R)-4-hydroxy-1-[(2R)-2-[3-(2-{4-[3-(2-hydroxyphenyl)-5-methylpyrrolo[3,2-c]pyridazin-6-yl]piperazin-1-yl}pyrimidin-5-yl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 28) and (2S,4R)-4-hydroxy-1-[(2S)-2-[3-(2-{4-[3-(2-hydroxyphenyl)-5-methylpyrrolo[3,2-c]pyridazin-6-yl]piperazin-1-yl}pyrimidin-5-yl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 29)

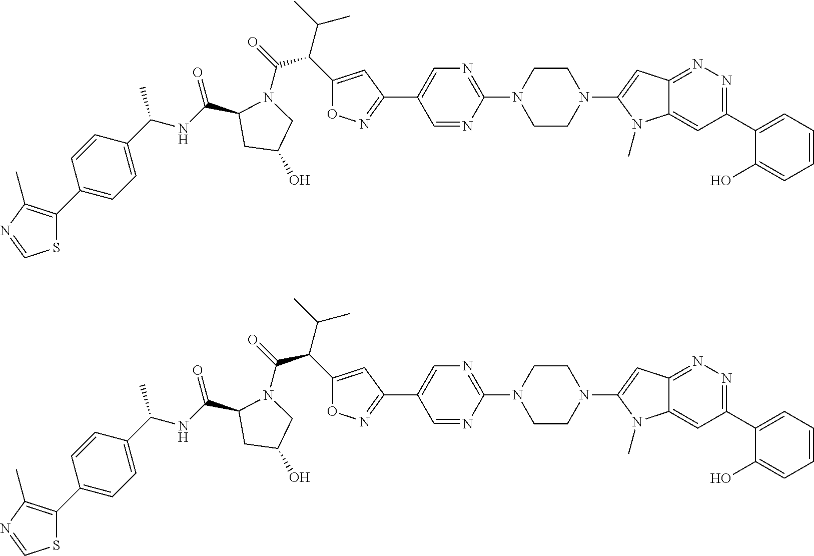 [SMILES: Cc1ncsc1-c1ccc([C@H](C)NC(=O)[C@@H]2C[C@@H](O)CN2C(=O)[C@@H](c2cc(-c3cnc(N4CCN(c5cc6nnc(-c7ccccc7O)cc6n5C)CC4)nc3)no2)C(C)C)cc1 ; Cc1ncsc1-c1ccc([C@H](C)NC(=O)[C@@H]2C[C@@H](O)CN2C(=O)[C@H](c2cc(-c3cnc(N4CCN(c5cc6nnc(-c7ccccc7O)cc6n5C)CC4)nc3)no2)C(C)C)cc1]

Intermediate 12 (120 mg) was purified by chiral HPLC with the following conditions: Column, CHIRAL ART Amylose-SA, 2*25 cm, 5 μm; Mobile Phase A: MtBE (10 mM NH₃-MeOH), Mobile Phase B: MeOH; Flow rate: 20 mL/min; Gradient: 20% B to 50% B in 16 min; Detector, UV 254/220 nm. This resulted in:

Compound 28 (35.40 mg, 29.50%) as a white solid. ¹H NMR (400 MHz, DMSO-d6) δ 14.82 (s, 1H), 8.99 (s, 1H), 8.89 (d, J=3.9 Hz, 2H), 8.44 (d, J=10.4 Hz, 2H), 8.15 (dd, J=8.9, 1.6 Hz, 1H), 7.47-7.42 (m, 2H), 7.41-7.35 (m, 2H), 7.34-7.26 (m, 1H), 7.02-6.92 (m, 3H), 6.33 (s, 1H), 5.23-5.01 (m, 1H), 5.01-4.69 (m, 1H), 4.39 (t, J=7.9 Hz, 1H), 3.35-3.25 (m, 1H), 4.08 (t, J=5.1 Hz, 4H), 3.86 (d, J=9.7 Hz, 1H), 3.80 (s, 3H), 3.79-3.72 (m, 1H), 3.69-3.47 (m, 1H), 3.32-3.26 (m, 4H), 2.45 (d, J=6.6 Hz, 3H), 2.39-2.18 (m, 1H), 2.09-1.99 (m, 1H), 1.86-1.75 (m, 1H), 1.39 (d, J=7.0 Hz, 3H), 1.02 (d, J=6.6 Hz, 3H), 0.85 (d, J=6.6 Hz, 3H). LCMS (ESI) m/z: [M+H]⁺=868.25.

Compound 29 (18.00 mg, 15.00%) as a white solid. ¹H NMR (400 MHz, DMSO-d6) δ 14.83 (s, 1H), 9.01-8.94 (m, 1H), 8.89 (s, 1H), 8.84 (s, 1H), 8.45 (s, 1H), 8.27 (d, J=7.9 Hz, 1H), 8.18-8.12 (m, 1H), 7.52-7.41 (m, 1H), 7.40-7.33 (m, 2H), 7.33-7.26 (m, 2H), 7.01-6.93 (m, 3H), 6.33 (d, J=0.8 Hz, 1H), 5.14 (d, J=3.7 Hz, 1H), 4.98-4.83 (m, 1H), 4.45 (t, J=7.7 Hz, 1H), 4.35-4.22 (m, 1H), 4.12-4.03 (m, 4H), 3.96 (d, J=8.9 Hz, 1H), 3.80 (d, J=1.7 Hz, 3H), 3.68-3.57 (m, 2H), 3.31-3.26 (m, 4H), 2.41 (s, 3H), 2.23-1.87 (m, 2H), 1.84-1.75 (m, 1H), 1.34 (d, J=7.0 Hz, 3H), 1.02 (d, J=6.6 Hz, 3H), 0.86 (t, J=6.6 Hz, 3H). LCMS (ESI) m/z: [M+H]⁺=868.25.

### Preparation of (2S,4R)-4-hydroxy-1-[(2R)-2-[3-(2-{4-hydroxy-4-[3-(2-hydroxyphenyl)-5-methylpyrrolo[3,2-c]pyridazin-6-yl]piperidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 30)

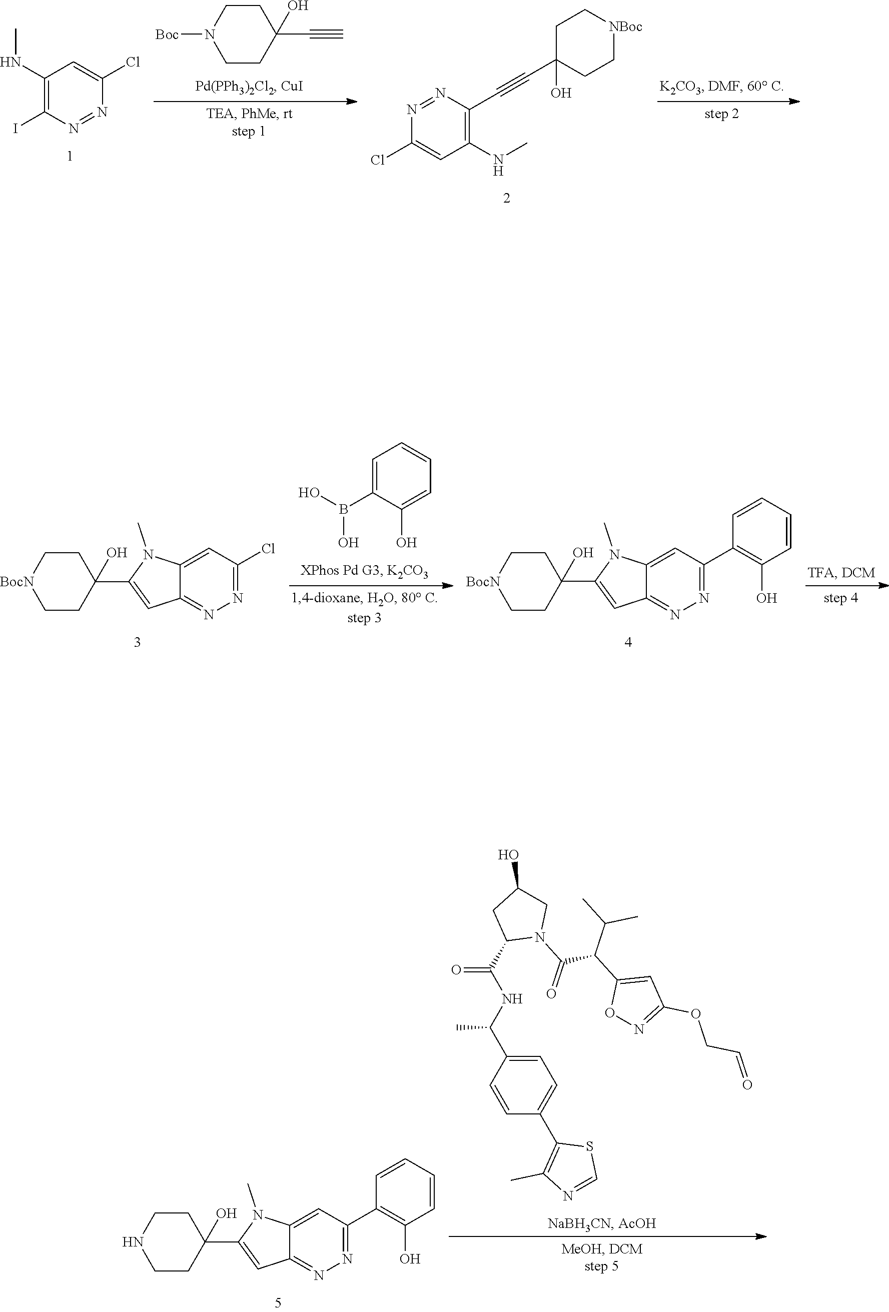

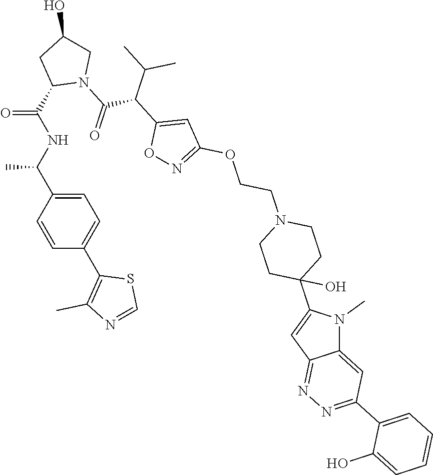 [SMILES: Cc1ncsc1-c1ccc([C@H](C)NC(=O)[C@@H]2C[C@@H](O)CN2C(=O)[C@@H](c2cc(OCCN3CCC(O)(c4cc5nnc(-c6ccccc6O)cc5n4C)CC3)no2)C(C)C)cc1]

### Step 1: Preparation of tert-butyl 4-([6-chloro-4-(methylamino)pyridazin-3-yl]ethynyl)-4-hydroxypiperidine-1-carboxylate (Intermediate 2)

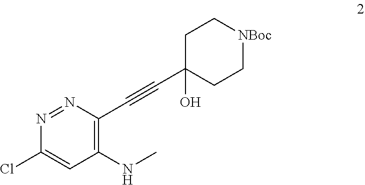 [SMILES: CNc1cc(Cl)nnc1C#CC1(O)CCN(C(=O)OC(C)(C)C)CC1]

To a solution of intermediate 1 (3.3 g, 12.246 mmol, 1 equiv), tert-butyl 4-ethynyl-4-hydroxypiperidine-1-carboxylate (2.76 g, 12.246 mmol, 1 equiv) and CuI (0.47 g, 2.449 mmol, 0.2 equiv) in toluene (30 mL) was added TEA (3.72 g, 36.738 mmol, 3 equiv). The resulting solution was stirred at room temperature for 4 hours. The resulting mixture was filtered and the filter cake was washed with DCM (3×50 mL). The filtrate was concentrated under reduced pressure. The residue was purified by silica gel column chromatography, eluted with PE/EA (2:1), to afford intermediate 2 (3.5 g, 77.91%) as a yellow solid. LCMS (ESI) m/z: [M+H]⁺=367.

### Step 2: Preparation of tert-butyl 4-{3-chloro-5-methylpyrrolo[3,2-c]pyridazin-6-yl}-4-hydroxypiperidine-1-carboxylate (Intermediate 3)

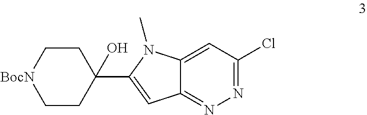 [SMILES: Cn1c(C2(O)CCN(C(=O)OC(C)(C)C)CC2)cc2nnc(Cl)cc21]

A mixture of intermediate 2 (3.5 g, 9.541 mmol, 1 equiv) and K₂CO₃ (2.64 g, 19.082 mmol, 2 equiv) in DMF (30 mL) was stirred at 60° C. for 16 hours. After cooling down to room temperature, the mixture was purified by reverse phase flash C18 chromatography, elution gradient 0% to 57% ACN in H₂O, to afford intermediate 3 (2.80 g, 80.00%) as a yellow solid. LCMS (ESI) m/z: [M+H]⁺=367.

### Step 3: Preparation of tert-butyl 4-hydroxy-4-[3-(2-hydroxyphenyl)-5-methylpyrrolo[3,2-c]pyridazin-6-yl]piperidine-1-carboxylate (intermediate 4)

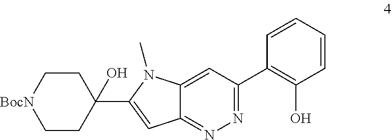 [SMILES: Cn1c(C2(O)CCN(C(=O)OC(C)(C)C)CC2)cc2nnc(-c3ccccc3O)cc21]

To a solution of intermediate 3 (2.80 g, 7.633 mmol, 1 equiv) and 2-hydroxyphenylboronic acid (1.58 g, 11.450 mmol, 1.5 equiv) in 1,4-dioxane (3.2 mL) and H₂O (0.8 mL) were added XPhos Pd G3 (0.65 g, 0.763 mmol, 0.1 equiv) and K₂CO₃ (2.11 g, 15.266 mmol, 2 equiv). The resulting mixture was stirred at 80° C. for 4 hours under nitrogen atmosphere. After cooling down to room temperature, the mixture was diluted with EtOAc (200 mL) and washed with water (3×150 mL). The organic layer was dried over anhydrous Na₂SO₄, filtered, and concentrated under reduced pressure. The residue was purified by silica gel column chromatography, eluted with PE/EA (2:1), to afford intermediate 4 (788 mg, 24.32%) as a yellow solid. LCMS (ESI) m/z: [M+H]⁺=425.

### Step 4: Preparation of 4-[3-(2-hydroxyphenyl)-5-methylpyrrolo[3,2-c]pyridazin-6-yl]piperidin-4-ol (Intermediate 5)

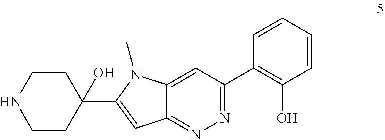 [SMILES: Cn1c(C2(O)CCNCC2)cc2nnc(-c3ccccc3O)cc21]

A solution of intermediate 4 (788 mg, 1.856 mmol, 1 equiv) in TFA (1.5 mL) and DCM (4.5 mL) was stirred at room temperature for 2 hours. The reaction mixture was concentrated under reduced pressure. The residue was purified by reverse phase flash C18 chromatography, elution gradient 0% to 35% ACN in H₂O, to afford intermediate 5 (374 mg, 62.11%) as a yellow solid. LCMS (ESI) m/z: [M+H]⁺=325.

### Step 5: Preparation of (2S,4R)-4-hydroxy-1-[(2R)-2-[3-(2-{4-hydroxy-4-[3-(2-hydroxyphenyl)-5-methylpyrrolo[3,2-c]pyridazin-6-yl]piperidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 30)

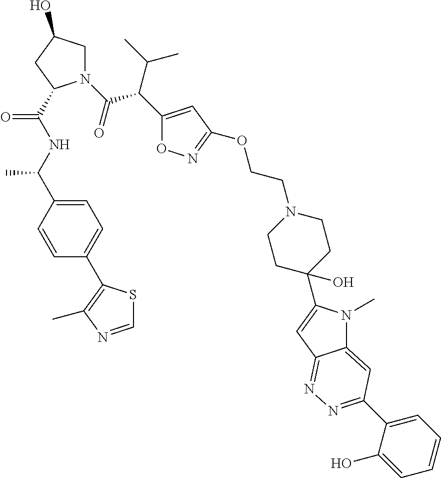 [SMILES: Cc1ncsc1-c1ccc([C@H](C)NC(=O)[C@@H]2C[C@@H](O)CN2C(=O)[C@@H](c2cc(OCCN3CCC(O)(c4cc5nnc(-c6ccccc6O)cc5n4C)CC3)no2)C(C)C)cc1]

A solution of intermediate 5 (20 mg, 0.062 mmol, 1 equiv) and (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (33.33 mg, 0.062 mmol, 1 equiv) in MeOH (0.8 mL) and DCM (0.8 mL) was stirred at room temperature for 30 minutes. NaBH₃CN (15.50 mg, 0.248 mmol, 4 equiv) and AcOH (0.37 mg, 0.006 mmol, 0.1 equiv) were then added and the mixture was stirred at room temperature for 6 hours. The resulting mixture was concentrated under reduced pressure. The residue was purified by Prep-HPLC with the following conditions: Column, XBridge Shield RP18 OBD, 19*150 mm, 5 μm; Mobile Phase A: water (10 mmol/L NH₄HCO₃), Mobile Phase B: CH₃CN; Flow rate: 25 mL/min; Gradient: 36% B to 50% B in 7 min, then 50% B; Detector, UV 254/220 nm. This resulted in Compound 30 (11.6 mg, 22.16%) as a white solid. ¹H NMR (300 MHz, DMSO-d6) δ 14.63 (s, 1H), 9.07 (s, 1H), 8.65 (s, 1H), 8.50 (d, J=7.5 Hz, 1H), 8.27 (d, J=8.0 Hz, 1H), 7.60-7.34 (m, 7H), 7.11-6.91 (m, 4H), 6.19 (s, 1H), 5.58 (s, 1H), 5.18 (d, J=3.7 Hz, 1H), 5.03-4.95 (s, 1H), 4.42 (s, 1H), 4.48-4.42 (m, 3H), 4.18 (s, 3H), 3.82-3.68 (m, 1H), 2.88-2.76 (m, 5H), 2.34 (s, 2H), 2.20-2.05 (m, 6H), 1.45 (d, J=6.9 Hz, 3H), 1.03 (d, J=6.5 Hz, 3H), 0.94-0.82 (m, 5H). LCMS (ESI) m/z: [M+H]⁺=849.10.

### Preparation of (2S,4R)-4-hydroxy-1-[(2R)-2-[3-(2-{3-[3-(2-hydroxyphenyl)pyrrolo[3,2-c]pyridazin-5-yl]azetidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 31)

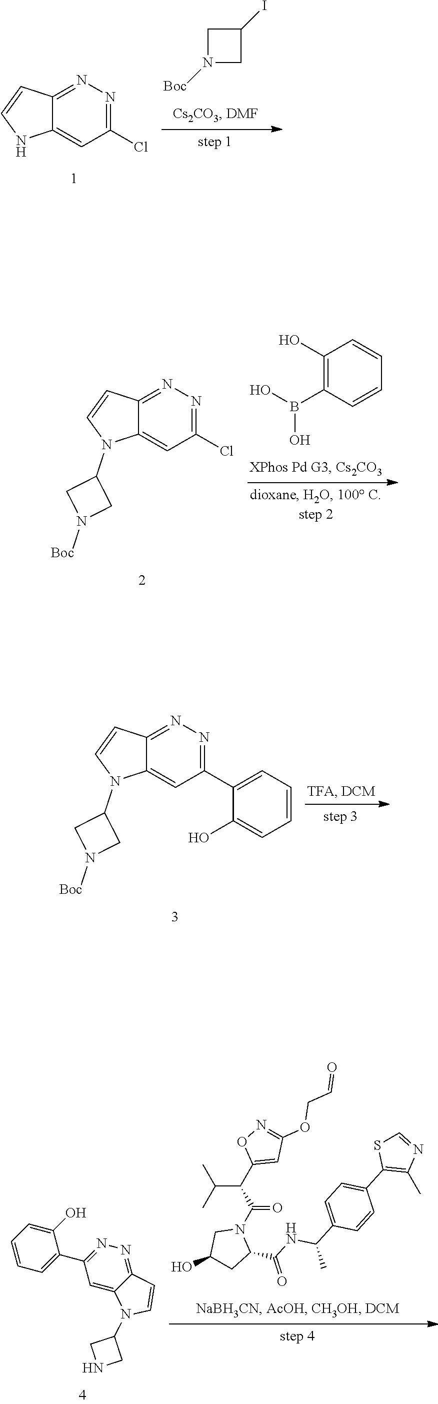

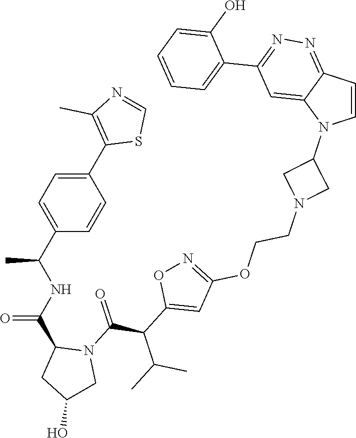 [SMILES: Cc1ncsc1-c1ccc([C@H](C)NC(=O)[C@@H]2C[C@@H](O)CN2C(=O)[C@@H](c2cc(OCCN3CC(n4ccc5nnc(-c6ccccc6O)cc54)C3)no2)C(C)C)cc1]

### Step 1: Preparation of tert-butyl 3-{3-chloropyrrolo[3,2-c]pyridazin-5-yl}azetidine-1-carboxylate (Intermediate 2)

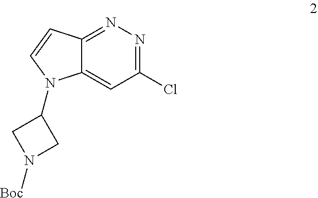 [SMILES: CC(C)(C)OC(=O)N1CC(n2ccc3nnc(Cl)cc32)C1]

A mixture of 3-chloro-5H-pyrrolo[3,2-c]pyridazine (200 mg, 1.302 mmol, 1.00 equiv), tert-butyl 3-iodoazetidine-1-carboxylate (737.41 mg, 2.604 mmol, 2.0 equiv) and Cs₂CO₃ (1.273 g, 3.906 mmol, 3 equiv) in DMF (4 mL) was stirred for 12 h at 90° C. under nitrogen atmosphere. After cooling down to room temperature, the mixture was purified by reverse phase flash chromatography with the following conditions: column, C18 silica gel; mobile phase, MeCN in water, 0% to 100% gradient in 40 min; detector, UV 254 nm. This resulted in Intermediate 2 (120 mg, 29.84%) as a yellow oil. LCMS (ESI) m/z [M+H]⁺=309.

### Step 2: Preparation of tert-butyl 3-[3-(2-hydroxyphenyl)pyrrolo[3,2-c]pyridazin-5-yl]azetidine-1-carboxylate (Intermediate 3)

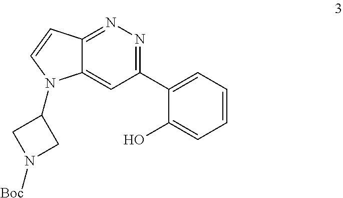 [SMILES: CC(C)(C)OC(=O)N1CC(n2ccc3nnc(-c4ccccc4O)cc32)C1]

To a solution of Intermediate 2 (120 mg, 0.389 mmol, 1.00 equiv) and 2-hydroxyphenylboronic acid (160.81 mg, 1.167 mmol, 3.0 equiv) in dioxane (5 mL) and H₂O (1 mL) were added Cs₂CO₃ (379.88 mg, 1.167 mmol, 3.0 equiv) and XPhos Pd G3 (65.79 mg, 0.078 mmol, 0.2 equiv). After stirring for 2 h at 100° C. under a nitrogen atmosphere, the resulting mixture was cooled down to room temperature and then was diluted with water (10 mL). The resulting mixture was extracted with EtOAc (3×20 mL). The combined organic layers were dried over anhydrous Na₂SO₄. After filtration, the filtrate was concentrated under reduced pressure. The residue was purified by Prep-TLC (PE/EA 1:2) to afford Intermediate 3 (100 mg, 70.22%) as a yellow oil. LCMS (ESI) m/z: [M+H]⁺=367.

### Step 3: Preparation of 2-[5-(azetidin-3-yl)pyrrolo[3,2-c]pyridazin-3-yl]phenol (Intermediate 4)

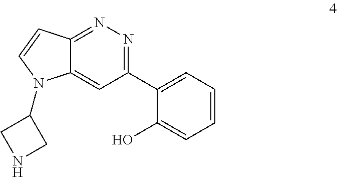 [SMILES: Oc1ccccc1-c1cc2c(ccn2C2CNC2)nn1]

A solution of Intermediate 3 (40 mg, 0.109 mmol, 1.00 equiv) and TFA (1 mL) in DCM (4 mL) was stirred for 2 h at room temperature. The resulting mixture was concentrated under reduced pressure. The residue was purified by Prep-HPLC with the following conditions: Column, Kinetex EVO C18, 21.2*150, 5 μm; Mobile Phase A: water (10 mmol/L NH₄HCO₃), Mobile Phase B: CH₃CN; Flow rate: 25 mL/min; Gradient: 14% B to 32% B in 6 min, then 32% B; Detector, UV 254/220 nm. This resulted in Intermediate 4 (16.5 mg, 56.36%) as a white solid. LCMS (ESI) m/z: [M+H]⁺=267.

### Step 4: Preparation of (2S,4R)-4-hydroxy-1-[(2R)-2-[3-(2-{3-[3-(2-hydroxyphenyl)pyrrolo[3,2-c]pyridazin-5-yl]azetidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 31)

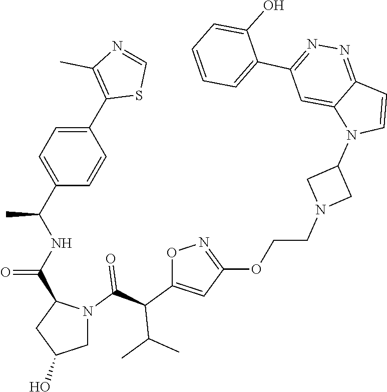 [SMILES: Cc1ncsc1-c1ccc([C@H](C)NC(=O)[C@@H]2C[C@@H](O)CN2C(=O)[C@@H](c2cc(OCCN3CC(n4ccc5nnc(-c6ccccc6O)cc54)C3)no2)C(C)C)cc1]

A solution of (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (40.60 mg, 0.075 mmol, 1.0 equiv) and Intermediate 4 (20 mg, 0.075 mmol, 1 equiv) in CH₃OH (1 mL) and DCM (1 mL) was stirred for 10 min at room temperature. To the above mixture were added AcOH (cat.) and NaBH₃CN (14.16 mg, 0.225 mmol, 3.0 equiv). The resulting mixture was stirred for additional 4 h at room temperature. The resulting mixture was concentrated under reduced pressure. The residue was purified by Prep-HPLC with the following conditions: Column, XBridge Prep C18 OBD, 19*150 mm, 5 μm; Mobile Phase A: water (10 mmol/L NH₄HCO₃), Mobile Phase B: CH₃CN; Flow rate: 25 mL/min; Gradient: 28% B to 57% B in 7 min; Detector, UV 254/220 nm. This resulted in Compound 31 (19.6 mg, 31.84%) as a white solid. ¹H NMR (300 MHz, DMSO-d6) δ 14.43 (s, 1H), 9.06 (s, 1H), 8.84 (s, 1H), 8.53-8.43 (m, 2H), 8.20 (d, J=8.3 Hz, 1H), 7.56-7.35 (m, 5H), 7.14 (t, J=3.9 Hz, 1H), 7.11-7.01 (m, 2H), 6.17 (s, 1H), 5.51 (t, J=6.6 Hz, 1H), 5.18 (d, J=3.6 Hz, 1H), 4.98 (t, J=7.2 Hz, 1H), 4.44 (t, J=8.0 Hz, 1H), 4.36 (brs, 1H), 4.29 (t, J=5.3 Hz, 2H), 3.95 (t, J=7.4 Hz, 2H), 3.82-3.69 (m, 2H), 3.63 (t, J=6.9 Hz, 2H), 3.57-3.48 (m, 1H), 3.04 (t, J=5.4 Hz, 2H), 2.53 (d, J=2.8 Hz, 3H), 2.37-2.24 (m, 1H), 2.17-2.04 (m, 1H), 1.92-1.78 (m, 1H), 1.48 (dd, J=21.3, 7.0 Hz, 3H), 1.03 (d, J=6.4 Hz, 3H), 0.87 (d, J=6.7 Hz, 3H). LCMS (ESI) m/z: [M+H]⁺=791.00.

### Preparation of (2S,4R)-1-((R)-2-(3-(2-(3-(7-(difluoromethyl)-3-(2-hydroxyphenyl)-5H-pyrrolo[3,2-c]pyridazin-5-yl)azetidin-1-yl)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide (Compound 32)

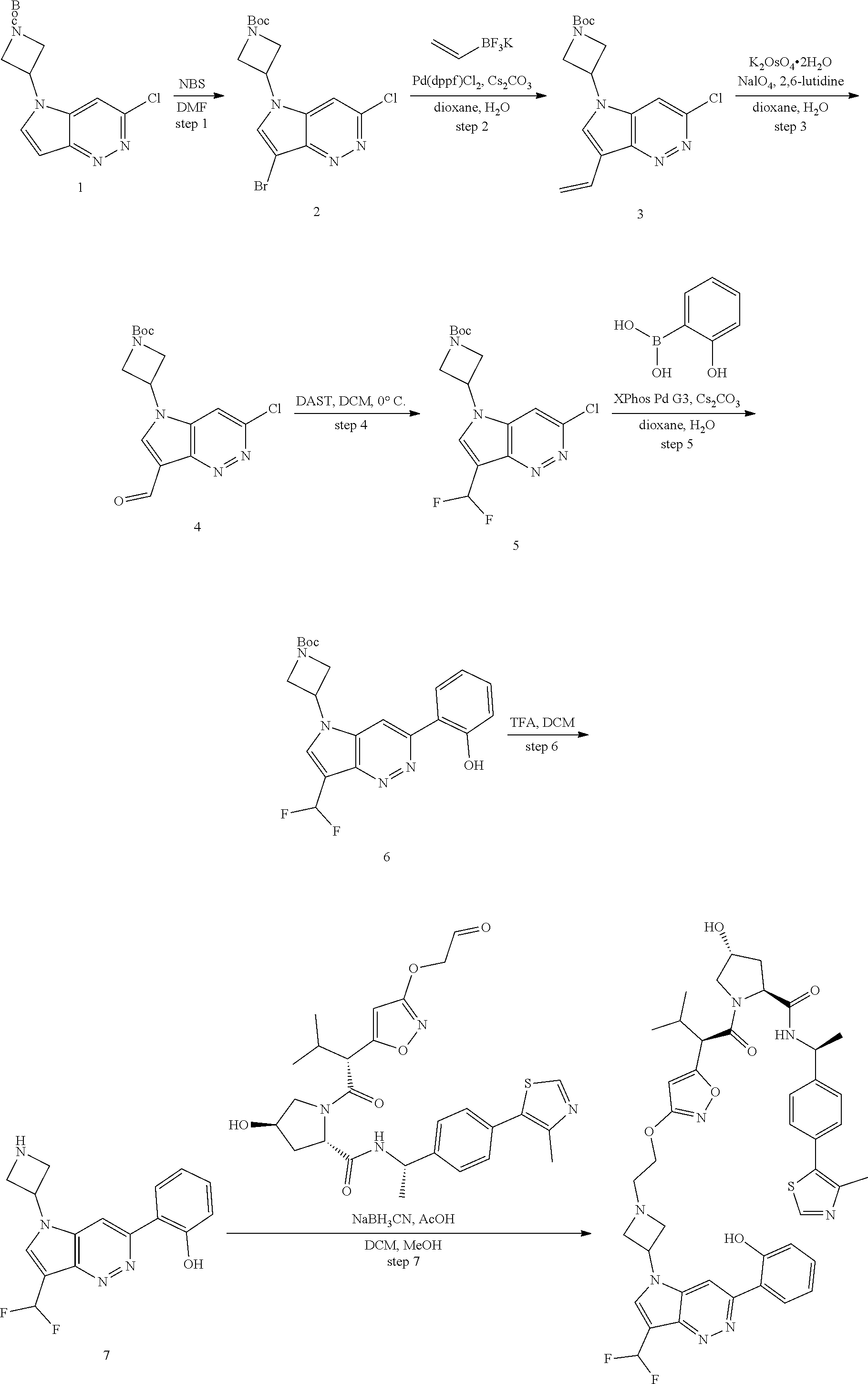

### Step 1: Preparation of tert-butyl 3-{7-bromo-3-chloropyrrolo[3,2-c]pyridazin-5-yl}azetidine-1-carboxylate (Intermediate 2)

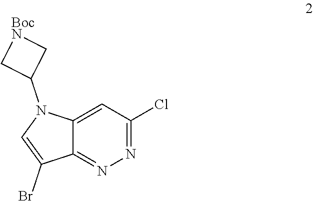 [SMILES: CC(C)(C)OC(=O)N1CC(n2cc(Br)c3nnc(Cl)cc32)C1]

A solution of intermediate 1 (927.0 mg, 3.002 mmol, 1.00 equiv) and NBS (534.3 mg, 3.002 mmol, 1.00 equiv) in DMF (10 mL) was stirred for 1 hour at room temperature. The mixture was purified by reverse phase flash chromatography with the following conditions: column, C18 silica gel; mobile phase, ACN in water, 0% to 100% gradient in 40 min; detector, UV 254 nm. This resulted in intermediate 2 (752.0 mg, 64.4%) as a yellow solid. LCMS (ESI) m/z: [M+H]⁺=387.

### Step 2: Preparation of tert-butyl 3-{3-chloro-7-ethenylpyrrolo[3,2-c]pyridazin-5-yl}azetidine-1-carboxylate (Intermediate 3)

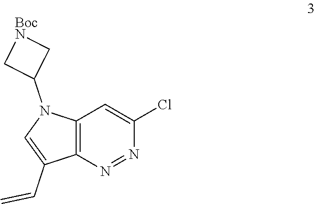 [SMILES: C=Cc1cn(C2CN(C(=O)OC(C)(C)C)C2)c2cc(Cl)nnc12]

To a stirred mixture of intermediate 2 (752.0 mg, 1.940 mmol, 1.00 equiv), Cs₂CO₃ (1.26 g, 3.880 mmol, 2.00 equiv) and Pd(dppf)Cl₂ (212.9 mg, 0.291 mmol, 0.15 equiv) in 1,4-dioxane (10 mL) and H₂O (2 mL) was added potassium (ethenyl)trifluoroborate (155.91 mg, 1.164 mmol, 0.6 equiv). The resulting mixture was stirred for 2 hours at 60° C. under nitrogen atmosphere. The resulting mixture was concentrated under reduced pressure. The residue was purified by reverse phase flash chromatography with the following conditions: column, C18 silica gel; mobile phase, ACN in water, 0% to 100% gradient in 40 min; detector, UV 254 nm. This resulted in intermediate 3 (453 mg, 69.70%) as a brown solid. LCMS (ESI) m/z: [M+H]⁺=335.

### Step 3: Preparation of tert-butyl 3-{3-chloro-7-formylpyrrolo[3,2-c]pyridazin-5-yl}azetidine-1-carboxylate (Intermediate 4)

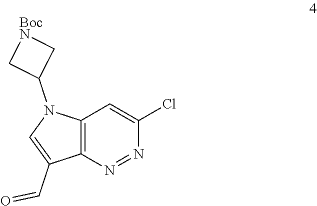 [SMILES: CC(C)(C)OC(=O)N1CC(n2cc(C=O)c3nnc(Cl)cc32)C1]

A solution of intermediate 3 (453.0 mg, 1.353 mmol, 1.00 equiv), NaIO₄ (2315.24 mg, 10.824 mmol, 8 equiv), K₂OsO₄·2H₂O (49.8 mg, 0.135 mmol, 0.10 equiv) and 2,6-lutidine (289.9 mg, 2.706 mmol, 2.00 equiv) in 1,4-dioxane (8 mL) and H₂O (8 mL) was stirred for 2 hours at 0° C. The resulting mixture was extracted with EA (3×10 mL). The combined organic layers were washed with brine (3×10 mL) then dried over anhydrous Na₂SO₄. After filtration, the filtrate was concentrated under reduced pressure. The residue was purified by reverse phase flash chromatography with the following conditions: column, C18 silica gel; mobile phase, MeCN in water, 0% to 100% gradient in 40 min; detector, UV 254 nm. This resulted in intermediate 4 (142.0 mg, 31.1%) as a brown solid. LCMS (ESI) m/z: [M+H]⁺=337.

### Step 4: Preparation of tert-butyl 3-[3-chloro-7-(difluoromethyl)pyrrolo[3,2-c]pyridazin-5-yl]azetidine-1-carboxylate (Intermediate 5)

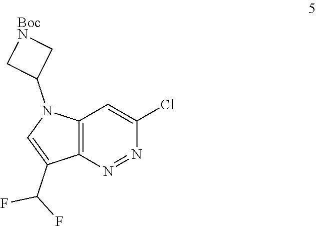 [SMILES: CC(C)(C)OC(=O)N1CC(n2cc(C(F)F)c3nnc(Cl)cc32)C1]

A solution of Intermediate 4 (142.0 mg, 0.422 mmol, 1.00 equiv) and DAST (1 mL, 15.22 equiv) in DCM (5 mL) was stirred for 3 hours at 0° C. under nitrogen atmosphere. The reaction was quenched with NH₄Cl at 0° C. The resulting mixture was extracted with EA (3×10 mL). The combined organic layers were washed with brine (3×4 mL) then dried over anhydrous Na₂SO₄. After filtration, the filtrate was concentrated under reduced pressure. The residue was purified by reverse phase flash chromatography with the following conditions: column, C18 silica gel; mobile phase, ACN in water, 0% to 100% gradient in 10 min; detector, UV 254 nm. This resulted in Intermediate 5 (15.0 mg, 9.9%) as a light yellow solid. LCMS (ESI) m/z: [M+H]⁺=359.

### Step 5: Preparation of tert-butyl 3-[7-(difluoromethyl)-3-(2-hydroxyphenyl)pyrrolo[3,2-c]pyridazin-5-yl]azetidine-1-carboxylate (Intermediate 6)

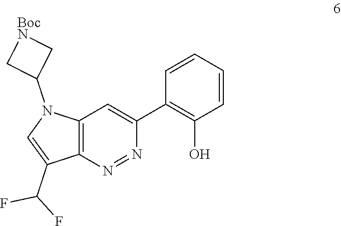 [SMILES: CC(C)(C)OC(=O)N1CC(n2cc(C(F)F)c3nnc(-c4ccccc4O)cc32)C1]

To a stirred mixture of intermediate 5 (15.0 mg, 0.044 mmol, 1.00 equiv), Cs₂CO₃ (35.6 mg, 0.110 mmol, 2.50 equiv) and 2-hydroxyphenylboronic acid (7.2 mg, 0.053 mmol, 1.20 equiv) in 1,4-dioxane (2 mL) and H₂O (0.4 mL) was added XPhos Pd G3 (7.4 mg, 0.009 mmol, 0.2 equiv). The resulting mixture was stirred for 2 hours at 80° C. under nitrogen atmosphere. The resulting mixture was concentrated under reduced pressure. The residue was purified by Prep-TLC (EA/PE 1:1) to afford intermediate 6 (10 mg, 54.50%) as a light yellow solid. LCMS (ESI) m/z: [M+H]⁺=417.

### Step 6: Preparation of 2-[5-(azetidin-3-yl)-7-(difluoromethyl)pyrrolo[3,2-c]pyridazin-3-yl]phenol (Intermediate 7)

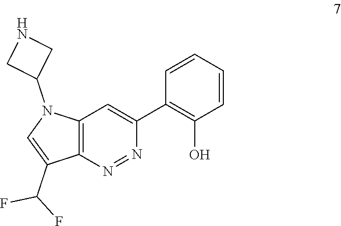 [SMILES: Oc1ccccc1-c1cc2c(nn1)c(C(F)F)cn2C1CNC1]

A solution of intermediate 6 (10.0 mg, 0.024 mmol, 1.00 equiv) in TFA (1 mL) and DCM (1 mL) was stirred for 1 hour at room temperature under nitrogen atmosphere. The resulting mixture was concentrated under reduced pressure to afford intermediate 7 (12 mg, crude) as a light yellow solid. LCMS (ESI) m/z: [M+H]⁺=317.

### Step 7: Preparation of (2S,4R)-1-((R)-2-(3-(2-(3-(7-(difluoromethyl)-3-(2-hydroxyphenyl)-5H-pyrrolo[3,2-c]pyridazin-5-yl)azetidin-1-yl)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide (Compound 32)

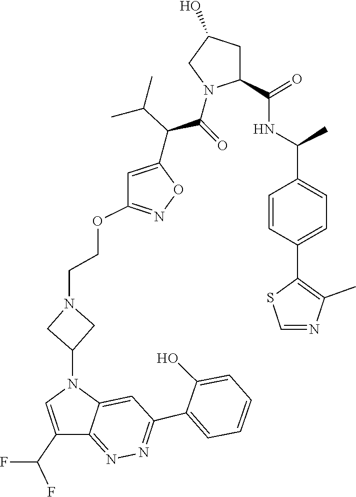 [SMILES: Cc1ncsc1-c1ccc([C@H](C)NC(=O)[C@@H]2C[C@@H](O)CN2C(=O)[C@@H](c2cc(OCCN3CC(n4cc(C(F)F)c5nnc(-c6ccccc6O)cc54)C3)no2)C(C)C)cc1]

A solution of intermediate 7 (8 mg, 0.015 mmol, 1 equiv), (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (9.36 mg, 0.030 mmol, 2 equiv) and AcOH (4.44 mg, 0.075 mmol, 5 equiv) in DCM (0.5 mL) and MeOH (0.5 mL) was stirred for 30 min at room temperature under nitrogen atmosphere. NaBH₃CN (2.32 mg, 0.037 mmol, 2.5 equiv) was then added at room temperature. The resulting mixture was stirred for 4 h at room temperature and then was concentrated under reduced pressure. The residue was purified by Prep-HPLC. This resulted in Compound 32 (4.7 mg, 36.18%) as a white solid. ¹H NMR (300 MHz, DMSO-d6) δ 13.89 (s, 1H), 8.99 (s, 1H), 8.85 (s, 1H), 8.79-8.73 (m, 1H), 8.42 (d, J=7.5 Hz, 1H), 8.13 (d, J=7.7 Hz, 1H), 7.49-7.32 (m, 6H), 7.02 (d, J=8.0 Hz, 2H), 6.10 (s, 1H), 5.45 (t, J=6.6 Hz, 1H), 5.11 (d, J=3.5 Hz, 1H), 4.92 (t, J=7.2 Hz, 1H), 4.43-4.18 (m, 4H), 3.87 (t, J=7.5 Hz, 2H), 3.72-3.56 (m, 4H), 3.49-3.39 (m, 1H), 2.97 (t, J=5.4 Hz, 2H), 2.46 (d, J=2.8 Hz, 3H), 2.37-2.24 (m, 1H), 2.17-2.04 (m, 1H), 1.92-1.78 (m, 1H), 1.41 (dd, J=22.1, 6.8 Hz, 3H), 0.96 (d, J=6.4 Hz, 3H), 0.80 (d, J=6.8 Hz, 3H). LCMS (ESI) m/z [M+H]⁺=841.2.

### Preparation of (2S,4R)-4-hydroxy-1-[(2R)-2-(3-{2-[(1R,5S,6S)-6-[3-(2-hydroxyphenyl)-6-methylpyrrolo[3,2-c]pyridazin-5-yl]-3-azabicyclo[3.1.0]hexan-3-yl]ethoxy}-1,2-oxazol-5-yl)-3-methylbutanoyl]-N-[(1S)-1-[4-(4-methyl-1,3-thiazol-5-yl)phenyl]ethyl]pyrrolidine-2-carboxamide (Compound 33)

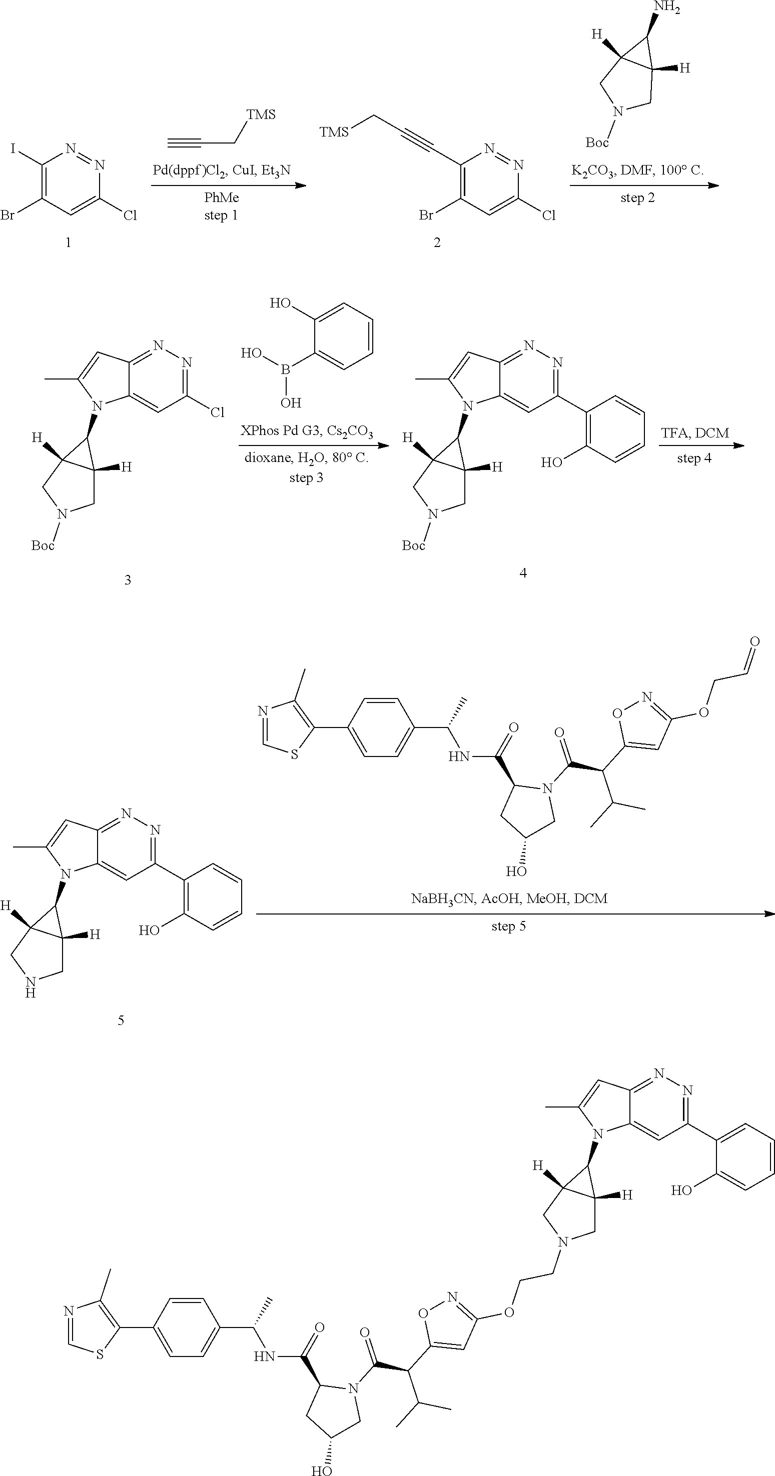

### Step 1: Preparation of 4-bromo-6-chloro-3-[3-(trimethylsilyl)prop-yl-yn-1-yl]pyridazine (Intermediate 2)

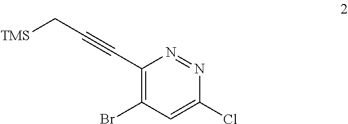 [SMILES: C[Si](C)(C)CC#Cc1nnc(Cl)cc1Br]

A mixture of Intermediate 1 (5 g, 15.658 mmol, 1 equiv), trimethyl(prop-2-yn-1-yl)silane (1.76 g, 15.658 mmol, 1 equiv), Pd(dppf)Cl₂·CH₂Cl₂ (1.27 g, 1.566 mmol, 0.1 equiv), CuI (596.42 mg, 3.132 mmol, 0.2 equiv) and Et₃N (3.17 g, 31.316 mmol, 2 equiv) in toluene (20 mL) was stirred overnight at room temperature under nitrogen atmosphere. The mixture was purified by reverse phase flash chromatography with the following conditions: column, C18 silica gel; mobile phase, MeCN in water, 0% to 100% gradient in 30 min; detector, UV 254 nm. This resulted in Intermediate 2 (827.5 mg, 17.40%) as a brown oil. LCMS (ESI) m/z [M+H]⁺=303.

### Step 2: Preparation of tert-butyl (1R,5S,6S)-6-{3-chloro-6-methylpyrrolo[3,2-c]pyridazin-5-yl}-3-azabicyclo[3.1.0]hexane-3-carboxylate (Intermediate 3)

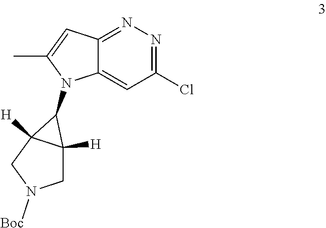 [SMILES: Cc1cc2nnc(Cl)cc2n1[C@H]1[C@@H]2CN(C(=O)OC(C)(C)C)C[C@@H]21]

A mixture of Intermediate 2 (400 mg, 1.317 mmol, 1 equiv), (1R,5S,6S)-tert-butyl 6-amino-3-azabicyclo[3.1.0]hexane-3-carboxylate (391 mg, 1.975 mmol, 1.5 equiv) and K₂CO₃ (546.16 mg, 3.951 mmol, 3 equiv) in DMF (5 mL) was stirred for 1 h at 100° C. under nitrogen atmosphere. After cooling down to room temperature, the mixture was purified by reverse phase flash chromatography with the following conditions: column, C18 silica gel; mobile phase, MeCN in water, 0% to 100% gradient in 30 min; detector, UV 254 nm. This resulted in Intermediate 3 (159 mg, 34.60%) as a brown solid. LCMS (ESI) m/z: [M+H]⁺=349.

### Step 3: Preparation of tert-butyl (1R,5S,6S)-6-[3-(2-hydroxyphenyl)-6-methylpyrrolo[3,2-c]pyridazin-5-yl]-3-azabicyclo[3.1.0]hexane-3-carboxylate (Intermediate 4)

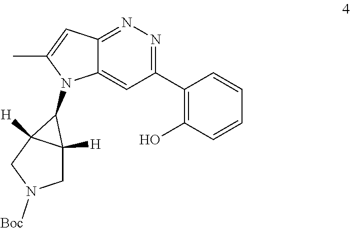 [SMILES: Cc1cc2nnc(-c3ccccc3O)cc2n1[C@H]1[C@@H]2CN(C(=O)OC(C)(C)C)C[C@@H]21]

A mixture of Intermediate 3 (65 mg, 0.186 mmol, 1 equiv), 2-hydroxyphenylboronic acid (77.10 mg, 0.558 mmol, 3 equiv), XPhos Pd G3 (31.55 mg, 0.037 mmol, 0.2 equiv) and Cs₂CO₃ (182.14 mg, 0.558 mmol, 3 equiv) in dioxane (3 mL) and H₂O (0.6 mL) was stirred for 1 h at 80° C. under nitrogen atmosphere. After cooling down to room temperature and concentration under reduced pressure, the mixture was purified by Prep-TLC (PE/EA 1:1) to afford Intermediate 4 (63 mg, 83.18%) as a yellow solid. LCMS (ESI) m/z: [M+H]⁺=407.

### Step 4: Preparation of 2-{5-[(1R,5S,6S)-3-azabicyclo[3.1.0]hexan-6-yl]-6-methylpyrrolo[3,2-c]pyridazin-3-yl}phenol (Intermediate 5)

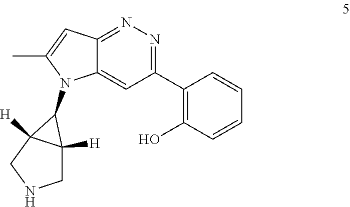 [SMILES: Cc1cc2nnc(-c3ccccc3O)cc2n1[C@H]1[C@@H]2CNC[C@@H]21]

A mixture of Intermediate 4 (63 mg, 0.155 mmol, 1 equiv) and TFA (1 mL) in DCM (3 mL) was stirred for 2 h at room temperature. The resulting mixture was concentrated under reduced pressure to afford Intermediate 5 (76.4 mg, TFA salt) as a yellow solid. LCMS (ESI) m/z: [M+H]⁺=307.

### Step 5: Preparation of (2S,4R)-4-hydroxy-1-[(2R)-2-(3-{2-[(1R,5S,6S)-6-[3-(2-hydroxyphenyl)-6-methylpyrrolo[3,2-c]pyridazin-5-yl]-3-azabicyclo[3.1.0]hexan-3-yl]ethoxy}-1,2-oxazol-5-yl)-3-methylbutanoyl]-N-[(1S)-1-[4-(4-methyl-1,3-thiazol-5-yl)phenyl]ethyl]pyrrolidine-2-carboxamide (Compound 33)

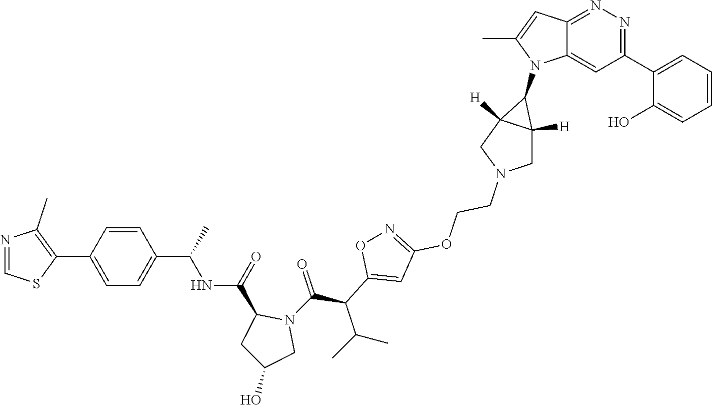 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4n3c(C)cc4nnc(-c5ccccc5O)cc43)no2)C(C)C)cc1]

A mixture of Intermediate 5 (20 mg, 0.065 mmol, 1.76 equiv), (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (20 mg, 0.037 mmol, 1.00 equiv) and AcOH (8.00 mg, 0.133 mmol, 3.60 equiv) in MeOH (1 mL) and DCM (1 mL) was stirred for 30 min at room temperature. To the above mixture was added NaBH₃CN (11.62 mg, 0.185 mmol, 5 equiv) at room temperature. The resulting mixture was stirred for additional 2 h at room temperature. The mixture was purified by Prep-HPLC with the following conditions: Column, XBridge Shield RP18 OBD, 19*150 mm, 5 μm; Mobile Phase A: water (10 mmol/L NH₄HCO₃), Mobile Phase B: CH₃CN; Flow rate: 25 mL/min; Gradient: 48% B to 73% B in 7 min; Detector, UV 254/220 nm. This resulted in Compound 33 (17.0 mg, 54.03%) as a white solid. ¹H NMR (300 MHz, DMSO-d6) δ 14.51 (s, 1H), 9.06 (s, 1H), 8.48 (d, J=7.4 Hz, 1H), 8.33 (s, 1H), 8.26 (d, J=8.0 Hz, 1H), 7.56-7.47 (m, 2H), 7.47-7.35 (m, 3H), 7.11-7.00 (m, 2H), 6.81 (s, 1H), 6.19 (s, 1H), 5.18 (d, J=3.5 Hz, 1H), 5.03-4.95 (m, 1H), 4.49-4.22 (m, 4H), 3.81-3.68 (m, 2H), 3.63-3.50 (m, 4H), 2.98-2.92 (m, 2H), 2.72-2.62 (m, 5H), 2.56-2.49 (m, 3H), 2.35-2.29 (m, 4H), 2.09 (s, 1H), 1.85 (s, 1H), 1.49 (dd, J=24.9, 6.9 Hz, 3H), 1.02 (d, J=6.7 Hz, 3H), 0.90-0.82 (m, 3H). LCMS (ESI) m/z: [M+H]⁺=831.50.

### Preparation of (2S,4R)-1-((R)-2-(3-(2-((S)-3-(5-cyclopropyl-3-(2-hydroxyphenyl)-5H-pyrrolo[3,2-c]pyridazin-6-yl)pyrrolidin-1-yl)pyrimidin-5-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 34) and (2S,4R)-1-((S)-2-(3-(2-((S)-3-(5-cyclopropyl-3-(2-hydroxyphenyl)-5H-pyrrolo[3,2-c]pyridazin-6-yl)pyrrolidin-1-yl)pyrimidin-5-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 35)

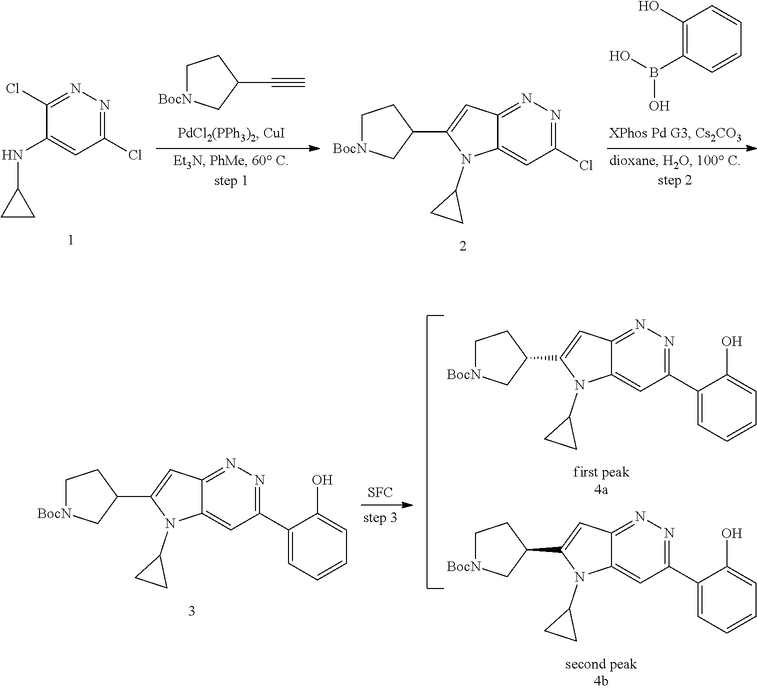

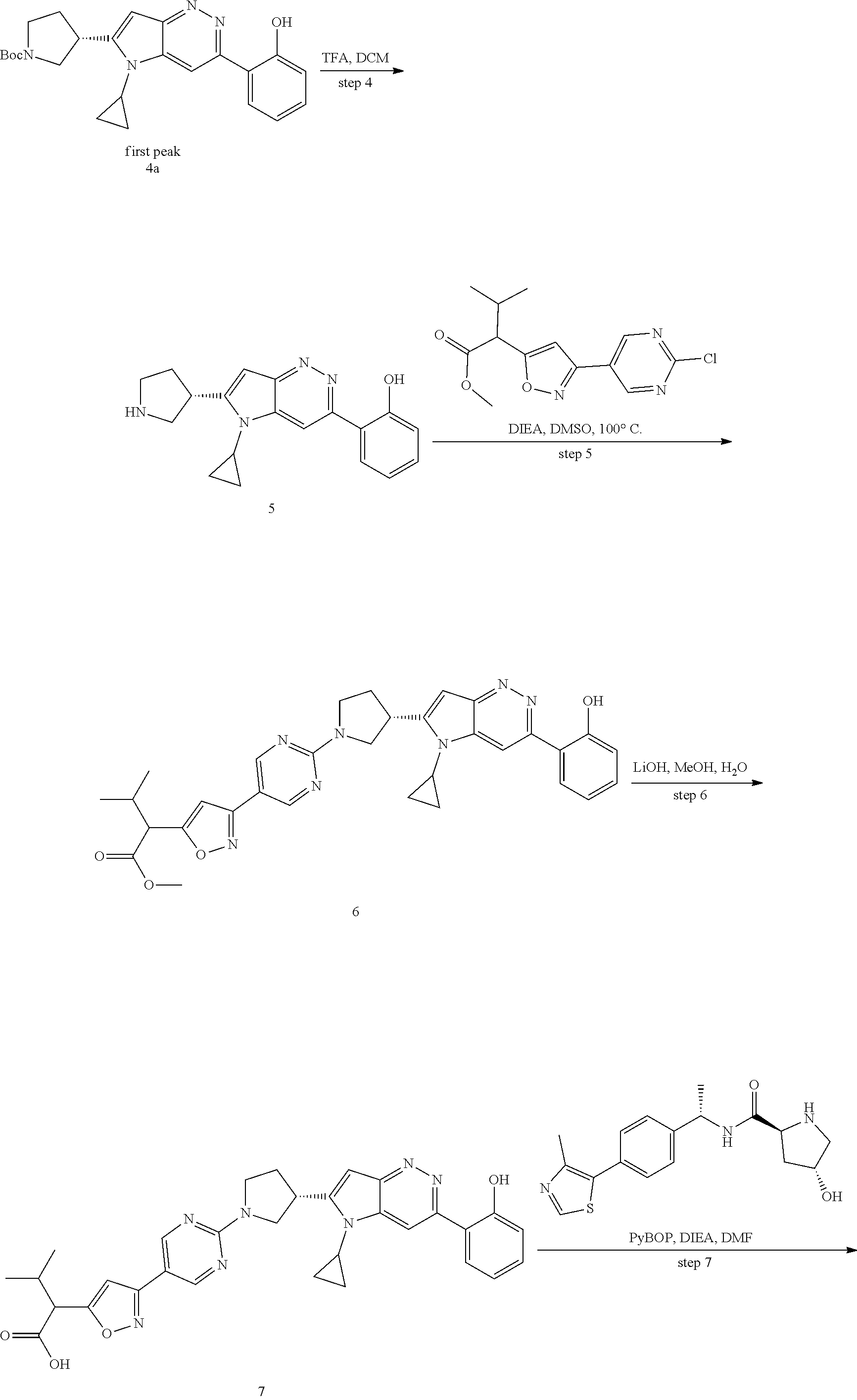

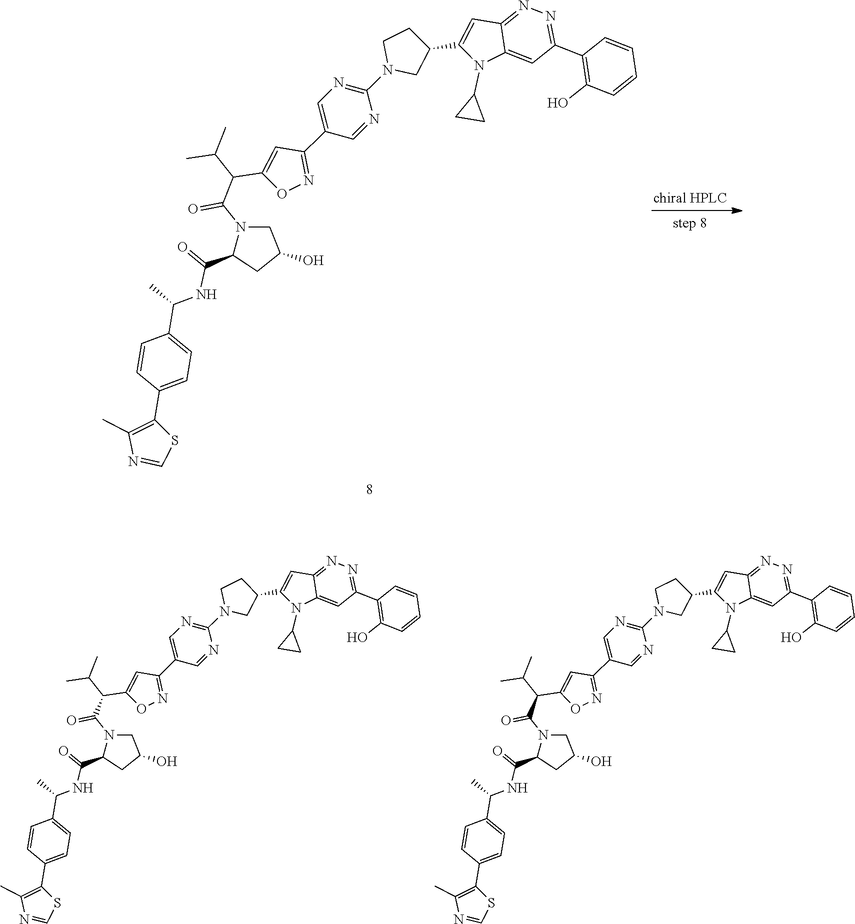 [SMILES: Cc1ncsc1-c1ccc([C@H](C)NC(=O)[C@@H]2C[C@@H](O)CN2C(=O)C(c2cc(-c3cnc(N4CC[C@H](c5cc6nnc(-c7ccccc7O)cc6n5C5CC5)C4)nc3)no2)C(C)C)cc1 ; Cc1ncsc1-c1ccc([C@H](C)NC(=O)[C@@H]2C[C@@H](O)CN2C(=O)[C@@H](c2cc(-c3cnc(N4CC[C@H](c5cc6nnc(-c7ccccc7O)cc6n5C5CC5)C4)nc3)no2)C(C)C)cc1 ; Cc1ncsc1-c1ccc([C@H](C)NC(=O)[C@@H]2C[C@@H](O)CN2C(=O)[C@H](c2cc(-c3cnc(N4CC[C@H](c5cc6nnc(-c7ccccc7O)cc6n5C5CC5)C4)nc3)no2)C(C)C)cc1]

### Step 1: Preparation tert-butyl 3-(3-chloro-5-cyclopropyl-5H-pyrrolo[3,2-c]pyridazin-6-yl)pyrrolidine-1-carboxylate (intermediate 2)

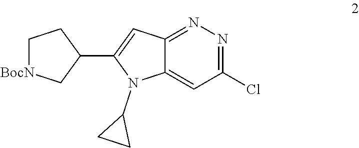 [SMILES: CC(C)(C)OC(=O)N1CCC(c2cc3nnc(Cl)cc3n2C2CC2)C1]

To a solution of intermediate 1 (3 g, 14.702 mmol, 1 equiv) in toluene (20 mL) were added tert-butyl 3-ethynylpyrrolidine-1-carboxylate (3.44 g, 17.642 mmol, 1.2 equiv), CuI (0.56 g, 2.940 mmol, 0.2 equiv), Pd(PPh₃)₂Cl₂ (2.06 g, 2.940 mmol, 0.2 equiv) and TEA (4.46 g, 44.106 mmol, 3 equiv) under nitrogen atmosphere. The resulting mixture was stirred at 60° C. for 2 hours. After cooling down to room temperature, the mixture was diluted with EtOAc (200 mL), then washed with brine (2×200 mL). The organic layer was dried over anhydrous Na2SO4. After filtration, the filtrate was concentrated under reduced pressure. The residue was purified by reverse phase flash C18 chromatography, elution gradient 0% to 35% CH3CN in water (0.05% FA), to give intermediate 2 (2.18 g, 40.86%) as a yellow oil. LCMS (ESI) m/z: [M+H]⁺=363.

### Step 2: Preparation of tert-butyl 3-(5-cyclopropyl-3-(2-hydroxyphenyl)-5H-pyrrolo[3,2-c]pyridazin-6-yl)pyrrolidine-1-carboxylate (intermediate 3)

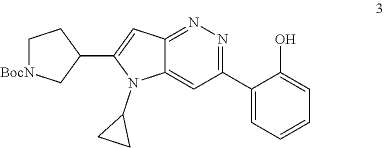 [SMILES: CC(C)(C)OC(=O)N1CCC(c2cc3nnc(-c4ccccc4O)cc3n2C2CC2)C1]

To a solution of intermediate 2 (2.5 g, 6.890 mmol, 1 equiv) and 2-hydroxyphenylboronic acid (1.43 g, 10.335 mmol, 1.5 equiv) in dioxane (16 mL) and H2O (4 mL) were added XPhos Pd G3 (583.19 mg, 0.689 mmol, 0.1 equiv) and Cs2CO3 (4.49 g, 13.780 mmol, 2 equiv) under nitrogen atmosphere. The resulting mixture was stirred at 100° C. for 1 hour. After cooling down to room temperature, the mixture was diluted with EtOAc (200 mL), then washed with brine (2×200 mL). The organic layer was dried over anhydrous Na2SO4. After filtration, the filtrate was concentrated under reduced pressure. The residue was purified by reverse phase flash C18 chromatography, elution gradient 0% to 65% CH3CN in water (0.05% FA), to give intermediate 3 (1.5 g, 51.77%) as a yellow oil. LCMS (ESI) m/z: [M+H]⁺=421.

### Step 3: Preparation of tert-butyl (S)-3-(5-cyclopropyl-3-(2-hydroxyphenyl)-5H-pyrrolo[3,2-c]pyridazin-6-yl)pyrrolidine-1-carboxylate (intermediate 4a) and tert-butyl (R)-3-(5-cyclopropyl-3-(2-hydroxyphenyl)-5H-pyrrolo[3,2-c]pyridazin-6-yl)pyrrolidine-1-carboxylate (intermediate 4b)

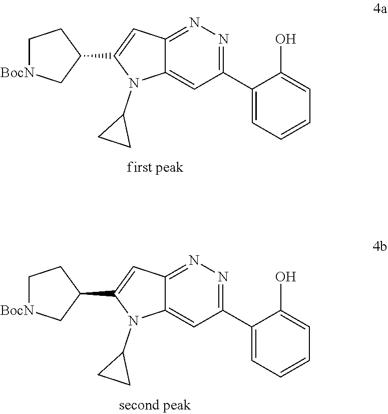 [SMILES: CC(C)(C)OC(=O)N1CC[C@H](c2cc3nnc(-c4ccccc4O)cc3n2C2CC2)C1 ; CC(C)(C)OC(=O)N1CC[C@@H](c2cc3nnc(-c4ccccc4O)cc3n2C2CC2)C1]

Intermediate 3 was purified by SFC-Prep-HPLC with the following conditions: Column, CHIRALPAK AS-H, 3*25 cm, 5 μm; Mobile Phase A: CO2, Mobile Phase B: MeOH; Flow rate: 65 mL/min; Gradient: isocratic 35% B; Column Temperature: 35° C.; Back Pressure: 100 bar; Detector, UV 254 nm; RT1=7.67 min; RT2=10.01 min; Sample Solvent: MeOH/DCM 1:1. This resulted in intermediate 4a (first peak) (797 mg) as a yellow solid and intermediate 4b (second peak) (639 mg) as a yellow solid.

### Step 4: Preparation of (S)-2-(5-cyclopropyl-6-(pyrrolidin-3-yl)-5H-pyrrolo[3,2-c]pyridazin-3-yl)phenol (intermediate 5)

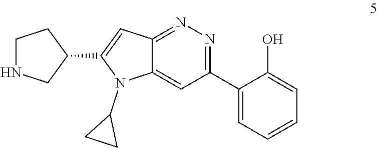 [SMILES: Oc1ccccc1-c1cc2c(cc([C@H]3CCNC3)n2C2CC2)nn1]

A solution of intermediate 4a (250 mg, 0.595 mmol, 1 equiv) in DCM (3 mL) and TFA (1 mL) was stirred at 25° C. for 1 hour. The resulting mixture was concentrated under reduced pressure to give intermediate 5 (300 mg, TFA salt) as a brown oil. LCMS (ESI) m/z: [M+H]⁺=321.

### Step 5: Preparation of methyl 2-(3-(2-((S)-3-(5-cyclopropyl-3-(2-hydroxyphenyl)-5H-pyrrolo[3,2-c]pyridazin-6-yl)pyrrolidin-1-yl)pyrimidin-5-yl)isoxazol-5-yl)-3-methylbutanoate (Intermediate 6)

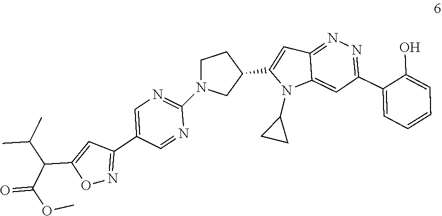 [SMILES: COC(=O)C(c1cc(-c2cnc(N3CC[C@H](c4cc5nnc(-c6ccccc6O)cc5n4C4CC4)C3)nc2)no1)C(C)C]

To a solution of intermediate 5 (300 mg, 0.936 mmol, 1 equiv) and methyl 2-[3-(2-chloropyrimidin-5-yl)-1,2-oxazol-5-yl]-3-methylbutanoate (276.89 mg, 0.936 mmol, 1 equiv) in DMSO (2 mL) was added DIEA (363.06 mg, 2.808 mmol, 3 equiv). The resulting solution was stirred at 100° C. for 2 hours. After cooling down to room temperature, the mixture was purified by reverse phase flash C18 chromatography, elution gradient 0% to 60% CH3CN in water (0.05% FA), to give intermediate 6 (265 mg, 48.82%) as a purple solid. LCMS (ESI) m/z: [M+H]⁺=580.

### Step 6: Preparation of 2-(3-(2-((S)-3-(5-cyclopropyl-3-(2-hydroxyphenyl)-5H-pyrrolo[3,2-c]pyridazin-6-yl)pyrrolidin-1-yl)pyrimidin-5-yl)isoxazol-5-yl)-3-methylbutanoic acid (Intermediate 7)

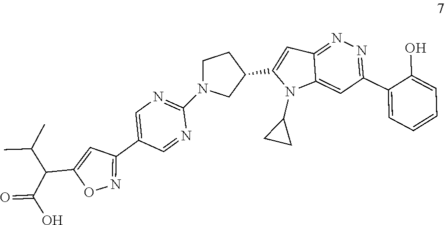 [SMILES: CC(C)C(C(=O)O)c1cc(-c2cnc(N3CC[C@H](c4cc5nnc(-c6ccccc6O)cc5n4C4CC4)C3)nc2)no1]

To a solution of intermediate 6 (265 mg, 0.457 mmol, 1 equiv) in MeOH (4 mL) and H₂O (1 mL) was added LiOH (54.75 mg, 2.285 mmol, 5 equiv). The resulting solution was stirred at room temperature for 2 hours. The mixture was acidified to pH 6 with HCl (1 M). The resulting mixture was extracted with EtOAc (3×20 mL). The combined organic layers were washed with brine (2×10 mL), then dried over anhydrous Na2SO4. After filtration, the filtrate was concentrated under reduced pressure to give intermediate 7 (214 mg, crude) as a brown solid. LCMS (ESI) m/z: [M+H]⁺=566.

### Step 7: Preparation of (2S,4R)-1-(2-(3-(2-((S)-3-(5-cyclopropyl-3-(2-hydroxyphenyl)-5H-pyrrolo[3,2-c]pyridazin-6-yl)pyrrolidin-1-yl)pyrimidin-5-yl)isoxazol-5-yl)-3-methylbutanoyl)-4-hydroxy-N—((S)-1-(4-(4-methylthiazol-5-yl)phenyl)ethyl)pyrrolidine-2-carboxamide (Intermediate 8)

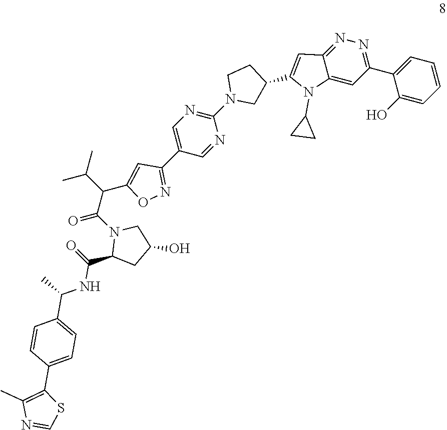 [SMILES: Cc1ncsc1-c1ccc([C@H](C)NC(=O)[C@@H]2C[C@@H](O)CN2C(=O)C(c2cc(-c3cnc(N4CC[C@H](c5cc6nnc(-c7ccccc7O)cc6n5C5CC5)C4)nc3)no2)C(C)C)cc1]

To a solution of intermediate 7 (214 mg, 0.378 mmol, 1 equiv) and (2S,4R)-4-hydroxy-N-[(1S)-1-[4-(4-methyl-1,3-thiazol-5-yl)phenyl]ethyl]pyrrolidine-2-carboxamide (125.39 mg, 0.378 mmol, 1.0 equiv) in DMF (2 mL) were added PyBOP (393.77 mg, 0.756 mmol, 2 equiv) and DIEA (244.49 mg, 1.890 mmol, 5 equiv) at room temperature. The resulting solution was stirred at room temperature for 2 hours. Without additional work-up, the reaction solution was purified by Prep-HPLC with the following conditions: Column, XBridge Shield RP18 OBD, 30*150 mm, 5 μm; Mobile Phase A: water (10 mmol/L NH4HCO3), Mobile Phase B: CH3CN; Flow rate: 60 mL/min; Gradient: 2% B for 1 min, 2% B to 40% B in 1.5 min, 40% B to 60% B in 9 min; Detector, UV 254/220 nm. This resulted in intermediate 8 (182 mg, 54.72%) as a yellow solid. LCMS (ESI) m/z: [M+H]⁺=879.

### Step 8: Preparation of (2S,4R)-1-((R)-2-(3-(2-((S)-3-(5-cyclopropyl-3-(2-hydroxyphenyl)-5H-pyrrolo[3,2-c]pyridazin-6-yl)pyrrolidin-1-yl)pyrimidin-5-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 34) and (2S,4R)-1-((S)-2-(3-(2-((S)-3-(5-cyclopropyl-3-(2-hydroxyphenyl)-5H-pyrrolo[3,2-c]pyridazin-6-yl)pyrrolidin-1-yl)pyrimidin-5-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 35)

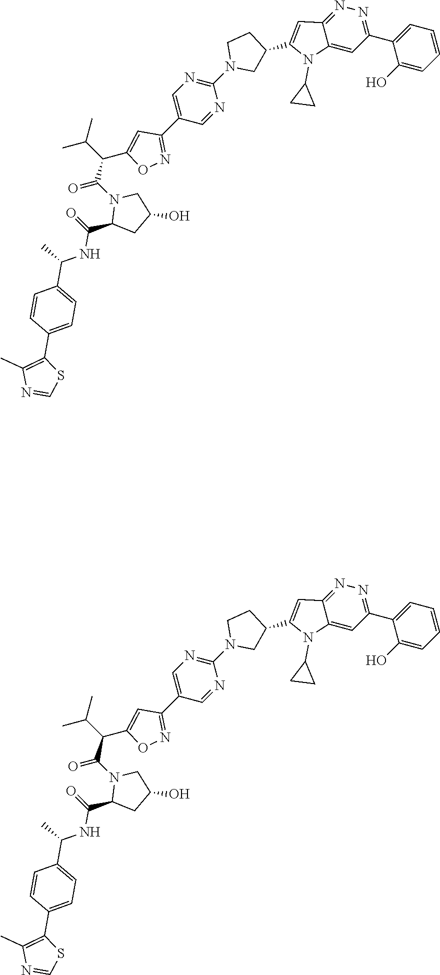 [SMILES: Cc1ncsc1-c1ccc([C@H](C)NC(=O)[C@@H]2C[C@@H](O)CN2C(=O)[C@@H](c2cc(-c3cnc(N4CC[C@H](c5cc6nnc(-c7ccccc7O)cc6n5C5CC5)C4)nc3)no2)C(C)C)cc1 ; Cc1ncsc1-c1ccc([C@H](C)NC(=O)[C@@H]2C[C@@H](O)CN2C(=O)[C@H](c2cc(-c3cnc(N4CC[C@H](c5cc6nnc(-c7ccccc7O)cc6n5C5CC5)C4)nc3)no2)C(C)C)cc1]

Intermediate 8 was purified by Chiral-Prep-HPLC with the following conditions: Column, CHIRALPAK IA-3, 4.6*50 mm, 3 μm; Mobile Phase A: MtBE (0.1% DIEA), Mobile Phase B: EtOH; Flow rate: 1 mL/min. This resulted in:

Compound 34 (57.9 mg, 17.36%) as a white solid. 1H NMR (400 MHz, DMSO-d6) δ 14.23 (s, 1H), 8.99 (d, J=2.0 Hz, 1H), 8.86 (s, 2H), 8.44 (d, J=7.7 Hz, 1H), 8.37 (s, 1H), 8.15 (d, J=7.8 Hz, 1H), 7.45 (d, J=8.2 Hz, 2H), 7.41-7.29 (m, 3H), 7.00 (t, J=7.9 Hz, 2H), 6.92 (d, J=10.4 Hz, 2H), 5.12 (s, 1H), 4.94 (h, J=6.9 Hz, 1H), 4.39 (t, J=7.9 Hz, 1H), 4.29 (dd, J=10.9, 6.9 Hz, 2H), 4.13 (p, J=7.8 Hz, 1H), 3.94-3.81 (m, 2H), 3.74 (td, J=11.8, 5.4 Hz, 2H), 3.54-3.44 (m, 2H), 2.69-2.58 (m, 2H), 2.52 (d, J=6.9 Hz, 3H), 2.45 (d, J=7.0 Hz, 2H), 2.10-1.99 (m, 1H), 1.80 (ddd, J=12.8, 8.0, 4.7 Hz, 1H), 1.50-1.28 (m, 5H), 1.21 (p, J=6.8, 6.2 Hz, 2H), 1.01 (d, J=6.2 Hz, 3H), 0.85 (t, J=7.2 Hz, 3H). LCMS (ESI) m/z: [M+H]⁺=879.25.

Compound 35 (43.9 mg, 13.11%) as a white solid. 1H NMR (400 MHz, DMSO-d6) δ 14.23 (d, J=6.2 Hz, 1H), 8.95 (s, 1H), 8.81 (s, 2H), 8.37 (s, 1H), 8.27 (d, J=7.9 Hz, 1H), 8.16 (d, J=7.9 Hz, 1H), 7.52-7.41 (m, 1H), 7.38-7.29 (m, 2H), 7.28 (d, J=8.1 Hz, 2H), 7.04-6.96 (m, 2H), 6.92-6.86 (m, 2H), 5.14 (d, J=3.6 Hz, 1H), 4.88 (t, J=7.3 Hz, 1H), 4.64 (t, J=7.7 Hz, 1H), 4.28 (t, J=9.2 Hz, 3H), 3.94 (t, J=7.8 Hz, 2H), 3.82 (d, J=9.0 Hz, 2H), 3.75 (s, 2H), 2.65 (s, 2H), 2.52 (dt, J=19.2, 10.3 Hz, 3H), 2.50-2.28 (m, 1H), 1.80 (dt, J=12.7, 6.2 Hz, 1H), 1.49 (d, J=6.9 Hz, 1H), 1.34 (dd, J=7.3, 3.6 Hz, 4H), 1.21 (s, 2H), 1.12 (d, J=6.6 Hz, 2H), 0.87 (t, J=6.4 Hz, 4H). LCMS (ESI) m/z: [M+H]⁺=879.25.

### Preparation of (2S,4R)-1-((R)-2-(3-(2-((R)-3-(5-cyclopropyl-3-(2-hydroxyphenyl)-5H-pyrrolo[3,2-c]pyridazin-6-yl)pyrrolidin-1-yl)pyrimidin-5-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 36) and (2S,4R)-1-((S)-2-(3-(2-((R)-3-(5-cyclopropyl-3-(2-hydroxyphenyl)-5H-pyrrolo[3,2-c]pyridazin-6-yl)pyrrolidin-1-yl)pyrimidin-5-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 37)

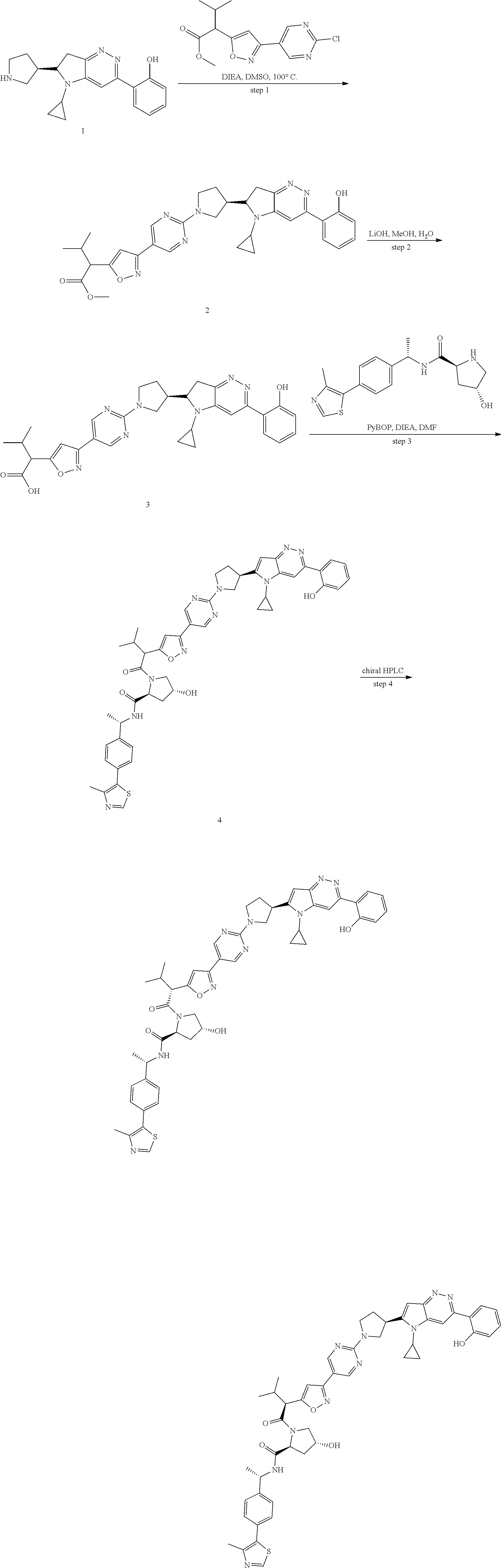

### Step 1: Preparation of methyl 2-(3-{2-[(3R)-3-[5-cyclopropyl-3-(2-hydroxyphenyl)pyrrolo[3,2-c]pyridazin-6-yl]pyrrolidin-1-yl]pyrimidin-5-yl}-1,2-oxazol-5-yl)-3-methylbutanoate (Intermediate 2)

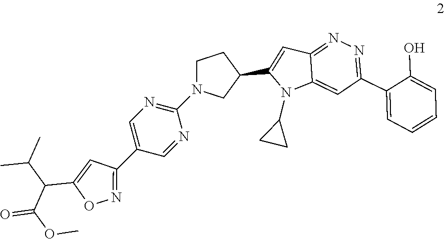 [SMILES: COC(=O)C(c1cc(-c2cnc(N3CC[C@@H](c4cc5nnc(-c6ccccc6O)cc5n4C4CC4)C3)nc2)no1)C(C)C]

A solution of compound 1 (350 mg, 1.092 mmol, 1 equiv) (prepared from intermediate 4b, second peak, using similar procedure to the one used above for the preparation of intermediate 5 from 4a, first peak), methyl 2-[3-(2-chloropyrimidin-5-yl)-1,2-oxazol-5-yl]-3-methylbutanoate (323.04 mg, 1.092 mmol, 1 equiv) and DIEA (423.57 mg, 3.276 mmol, 3 equiv) in DMSO (3 mL) was stirred at 100° C. for 3 h. After cooling down to room temperature, the mixture was purified by reverse phase flash chromatography with the following conditions: column, C18 silica gel; mobile phase, CH3CN in water (0.05% FA), 0% to 60% gradient in 30 min. This resulted in intermediate 2 (218 mg, 34.43%) as a yellow solid. LCMS (ESI) m/z: [M+H]⁺=580.

### Step 2: Preparation of 2-(3-{2-[(3R)-3-[5-cyclopropyl-3-(2-hydroxyphenyl)pyrrolo[3,2-c]pyridazin-6-yl]pyrrolidin-1-yl]pyrimidin-5-yl}-1,2-oxazol-5-yl)-3-methylbutanoic acid (intermediate 3)

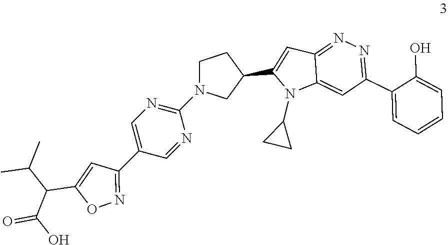 [SMILES: CC(C)C(C(=O)O)c1cc(-c2cnc(N3CC[C@@H](c4cc5nnc(-c6ccccc6O)cc5n4C4CC4)C3)nc2)no1]

A solution of intermediate 2 (218 mg, 0.376 mmol, 1 equiv) and LiOH (45.04 mg, 1.880 mmol, 5 equiv) in MeOH (4 mL) and H2O (2 mL) was stirred at room temperature for 4 hours. The mixture was acidified to pH 6 with HCl (1 M). The resulting mixture was extracted with EA (2×200 mL). The combined organic layers were washed with brine (200 mL), then dried over anhydrous Na2SO4. After filtration, the filtrate was concentrated under reduced pressure. This resulted in intermediate 3 (230 mg, crude) as a brown solid. LCMS (ESI) m/z: [M+H]⁺=566.

### Step 3: Preparation of (2S,4R)-1-(2-(3-(2-((R)-3-(5-cyclopropyl-3-(2-hydroxyphenyl)-5H-pyrrolo[3,2-c]pyridazin-6-yl)pyrrolidin-1-yl)pyrimidin-5-yl)isoxazol-5-yl)-3-methylbutanoyl)-4-hydroxy-N—((S)-1-(4-(4-methylthiazol-5-yl)phenyl)ethyl)pyrrolidine-2-carboxamide (intermediate 4)

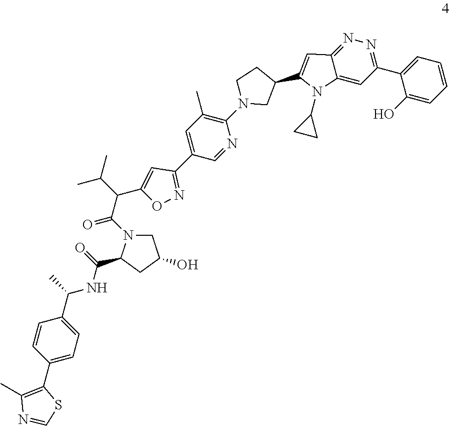 [SMILES: Cc1cc(-c2cc(C(C(=O)N3C[C@H](O)C[C@H]3C(=O)N[C@@H](C)c3ccc(-c4scnc4C)cc3)C(C)C)on2)cnc1N1CC[C@@H](c2cc3nnc(-c4ccccc4O)cc3n2C2CC2)C1]

A solution of intermediate 3 (230 mg, 0.407 mmol, 1 equiv), (2S,4R)-4-hydroxy-N-[(1S)-1-[4-(4-methyl-1,3-thiazol-5-yl)phenyl]ethyl]pyrrolidine-2-carboxamide (269.53 mg, 0.814 mmol, 2 equiv), PyBOP (423.21 mg, 0.814 mmol, 2 equiv) and DIEA (262.77 mg, 2.035 mmol, 5 equiv) in DMF (3 mL) was stirred at room temperature for 2 h. Without additional work-up, the reaction solution was purified by Prep-HPLC with the following conditions: Column, XBridge Shield RP18 OBD, 30*150 mm, 5 μm; Mobile Phase A: water (10 mmol/L NH4HCO3), Mobile Phase B: CH3CN; Flow rate: 60 mL/min; Gradient: 2% B for 1 min, 2% B to 38% B in 1.5 min, 38% B to 60% B in 8 min, 60% B; Detector, UV 254/220 nm. This resulted in intermediate 4 (200 mg, 56.02%) as a yellow solid. LCMS (ESI) m/z: [M+H]⁺=879.

### Step 4: Preparation of (2S,4R)-1-((R)-2-(3-(2-((R)-3-(5-cyclopropyl-3-(2-hydroxyphenyl)-5H-pyrrolo[3,2-c]pyridazin-6-yl)pyrrolidin-1-yl)pyrimidin-5-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 36) and (2S,4R)-1-((S)-2-(3-(2-((R)-3-(5-cyclopropyl-3-(2-hydroxyphenyl)-5H-pyrrolo[3,2-c]pyridazin-6-yl)pyrrolidin-1-yl)pyrimidin-5-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 37)

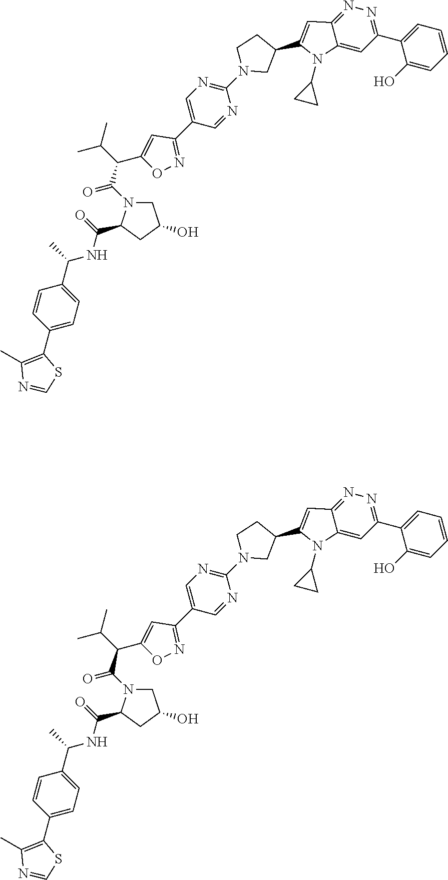 [SMILES: Cc1ncsc1-c1ccc([C@H](C)NC(=O)[C@@H]2C[C@@H](O)CN2C(=O)[C@@H](c2cc(-c3cnc(N4CC[C@@H](c5cc6nnc(-c7ccccc7O)cc6n5C5CC5)C4)nc3)no2)C(C)C)cc1 ; Cc1ncsc1-c1ccc([C@H](C)NC(=O)[C@@H]2C[C@@H](O)CN2C(=O)[C@H](c2cc(-c3cnc(N4CC[C@@H](c5cc6nnc(-c7ccccc7O)cc6n5C5CC5)C4)nc3)no2)C(C)C)cc1]

Intermediate 4 (200 mg) was purified by Chiral-Prep-HPLC with the following conditions: Column, CHIRAL ART Amylose-SA, 3*25 cm, 5 μm; Mobile phase, MtBE (0.5% 2 M NH3-MeOH)/EtOH (hold 50% EtOH for 52 mi). This resulted in:

Compound 36 (82.8 mg) as white solid. 1H NMR (400 MHz, DMSO-d6) w 14.24 (d, J=3.7 Hz, 1H), 8.99 (d, J=1.5 Hz, 1H), 8.86 (s, 2H), 8.44 (d, J=7.7 Hz, 1H), 8.37 (s, 1H), 8.15 (d, J=8.3 Hz, 1H), 7.48-7.43 (m, 2H), 7.41-7.29 (m, 3H), 7.02 (t, J=7.7 Hz, 2H), 7.00-6.89 (m, 2H), 5.12 (d, J=3.6 Hz, 1H), 4.94 (q, J=6.8 Hz, 1H), 4.39 (t, J=7.9 Hz, 1H), 4.29 (t, J=9.3 Hz, 2H), 4.13 (p, J=7.8 Hz, 1H), 3.94-3.83 (m, 2H), 3.81-3.58 (m, 3H), 3.54-3.44 (m, 2H), 2.62 (s, 1H), 2.45 (d, J=6.5 Hz, 3H), 2.38-2.26 (m, 2H), 2.10-1.99 (m, 1H), 1.80 (dd, J=12.6, 4.7 Hz, 1H), 1.49 (d, J=7.0 Hz, 3H), 1.36 (dd, J=19.3, 7.2 Hz, 2H), 1.19 (d, J=6.5 Hz, 2H), 1.02 (dd, J=6.6, 4.0 Hz, 3H), 0.85 (t, J=7.2 Hz, 3H). LCMS (ESI) m/z: [M+H]⁺=879.25.

Compound 37 (47.3 mg) as white solid. 1H NMR (400 MHz, DMSO-d6) δ 14.24 (d, J=6.1 Hz, 1H), 8.98 (d, J=16.9 Hz, 1H), 8.86 (s, 2H), 8.37 (s, 1H), 8.27 (d, J=7.9 Hz, 1H), 8.16 (d, J=8.0 Hz, 1H), 7.65-7.58 (m, 1H), 7.52-7.41 (m, 2H), 7.38-7.25 (m, 2H), 7.04-6.95 (m, 2H), 6.88-6.75 (m, 2H), 5.14 (d, J=3.6 Hz, 2H), 5.08-4.93 (m, 1H), 4.88 (p, J=7.0 Hz, 2H), 4.44 (t, J=7.8 Hz, 1H), 4.32-4.23 (m, 1H), 4.14 (p, J=7.5 Hz, 1H), 3.81-3.69 (m, 2H), 3.63 (s, 1H), 3.57-3.44 (m, 2H), 2.63 (s, 1H), 2.47 (s, 1H), 2.40 (s, 2H), 2.35 (d, J=6.6 Hz, 2H), 2.12-2.02 (m, 1H), 1.85-1.73 (m, 1H), 1.49 (d, J=7.0 Hz, 1H), 1.34 (d, J=7.1 Hz, 4H), 1.20 (d, J=9.0 Hz, 2H), 1.01 (d, J=6.6 Hz, 2H), 0.91-0.78 (m, 4H). LCMS (ESI) m/z: [M+H]⁺=879.25.

The compounds in Table 8 were prepared using procedures similar to the one used above for the preparation of Compound 37 using the appropriate amines.

TABLE 8

| | | LCMS (ESI) | |
|---|---|---|---|
| No. | Name | m/z | ¹H NMR |
| 89 | (2S,4R)-1-((R)-2-(3-(2-((R)-3-(5- | 865.3 | ¹H NMR (300 MHz, Methanol-d4) δ 8.94-8.60 |
| | cyclopropyl-3-(2-hydroxyphenyl)- | | (m, 3H), 8.26 (d, J = 3.3 Hz, 1H), 7.95 (dt, J = |
| | 5H-pyrrolo[3,2-c]pyridazin-6- | | 8.3, 2.1 Hz, 1H), 7.43 (d, J = 10.8 Hz, 4H), 7.29 |
| | yl)pyrrolidin-1-yl)pyrimidin-5- | | (td, J = 7.6, 7.1, 1.5 Hz, 1H), 7.04-6.88 (m, |
| | yl)isoxazol-5-yl)-3-methylbutanoyl)- | | 2H), 6.74 (d, J = 21.0 Hz, 2H), 4.65-4.39 (m, |
| | 4-hydroxy-N-(4-(4-methylthiazol-5- | | 4H), 4.30 (dd, J = 10.9, 7.5 Hz, 1H), 4.14 (q, |
| | yl)benzyl)pyrrolidine-2- | | J = 7.4 Hz, 1H), 4.01-3.51 (m, 6H), 3.43 (tt, J = |
| | carboxamide | | 7.1, 3.8 Hz, 1H), 2.67 (dd, J = 12.0, 5.8 Hz, |
| | | | 1H), 2.48 (s, 3H), 2.41-2.29 (m, 2H), 2.21 (d, |
| | | | J = 9.0 Hz, 1H), 2.09 (ddd, J = 13.2, 8.7, 4.6 |
| | | | Hz, 1H), 1.40 (dt, J = 7.0, 3.2 Hz, 2H), 1.31- |
| | | | 1.18 (m, 3H), 1.09 (d, J = 6.5 Hz, 3H), 0.92 (d, |
| | | | J = 6.7 Hz, 3H). |
| 90 | (2S,4R)-1-((R)-2-(3-(2-((S)-3-(5- | 865.25 | ¹H NMR (400 MHz, DMSO-d6) δ 14.24 (s, 1H), |
| | cyclopropyl-3-(2-hydroxyphenyl)- | | 8.98 (d, J = 11.2 Hz, 1H), 8.86 (s, 2H), 8.55 (t, |
| | 5H-pyrrolo[3,2-c]pyridazin-6- | | J = 6.0 Hz, 1H), 8.36 (s, 1H), 8.19-8.12 (m, |
| | yl)pyrrolidin-1-yl)pyrimidin-5- | | 1H), 7.46-7.31 (m, 5H), 7.04-6.95 (m, 2H), |
| | yl)isoxazol-5-yl)-3-methylbutanoyl)- | | 6.92 (d, J = 11.8 Hz, 2H), 5.13 (d, J = 3.7 Hz, |
| | 4-hydroxy-N-(4-(4-methylthiazol-5- | | 1H), 4.49-4.21 (m, 5H), 4.19-4.07 (m, 1H), |
| | yl)benzyl)pyrrolidine-2- | | 3.96-3.76 (m, 3H), 3.77-3.68 (m, 2H), 3.55- |
| | carboxamide | | 3.44 (m, 2H), 2.67-2.58 (m, 1H), 2.43 (d, J = |
| | | | 25.1 Hz, 3H), 2.40-2.24 (m, 2H), 2.10- |
| | | | 1.98 (m, 1H), 1.97-1.87 (m, 1H), 1.37-1.31 |
| | | | (m, 2H), 1.26-1.18 (m, 2H), 1.00 (d, J = 6.5 |
| | | | Hz, 3H), 0.84 (d, J = 6.7 Hz, 3H). |
| 92 | (2S,4R)-1-((R)-2-(3-(2-((S)-3-(5- | 800.35 | ¹H NMR (400 MHz, DMSO-d6) δ 14.22 (s, 1H), |
| | cyclopropyl-3-(2-hydroxyphenyl)- | | 8.85 (s, 2H), 8.35 (d, J = 5.8 Hz, 2H), 8.15 (d, |
| | 5H-pyrrolo[3,2-c]pyridazin-6- | | J = 8.0 Hz, 1H), 7.44-7.27 (m, 3H), 7.13 (t, J = |
| | yl)pyrrolidin-1-yl)pyrimidin-5- | | 8.9 Hz, 2H), 7.00 (t, J = 8.0 Hz, 2H), 6.91 (d, J = |
| | yl)isoxazol-5-yl)-3-methylbutanoyl)- | | 7.1 Hz, 2H), 5.09 (d, J = 3.7 Hz, 1H), 4.88 (q, |
| | N-((S)-1-(4-fluorophenyl)ethyl)-4- | | J = 7.4 Hz, 1H), 4.36-4.24 (m, 3H), 4.17- |
| | hydroxypyrrolidine-2-carboxamide | | 4.09 (m, 1H), 3.94-3.89 (m, 1H), 3.87-3.82 |
| | | | (m, 1H), 3.78-3.67 (m, 3H), 3.50 (s, 2H), 2.69- |
| | | | 2.63 (m, 2H), 2.35 (d, J = 8.2 Hz, 2H), 2.00 |
| | | | (dd, J = 12.9, 7.8 Hz, 1H), 1.81-1.70 (m, 1H), |
| | | | 1.34 (dd, J = 7.1, 2.7 Hz, 4H), 1.16-1.11 (m, |
| | | | 2H), 1.01 (d, J = 6.5 Hz, 3H), 0.84 (t, J = 6.7 Hz, |
| | | | 3H). |
| 93 | (2S,4R)-1-((R)-2-(3-(2-((S)-3-(5- | 783.3 | ¹H NMR (400 MHz, DMSO-d6) δ 14.23 (s, 1H), |
| | cyclopropyl-3-(2-hydroxyphenyl)- | | 8.86 (s, 2H), 8.37 (d, J = 5.7 Hz, 2H), 8.15 (d, |
| | 5H-pyrrolo[3,2-c]pyridazin-6- | | J = 8.0 Hz, 1H), 7.42-7.18 (m, 6H), 7.00 (t, J = |
| | yl)pyrrolidin-1-yl)pyrimidin-5- | | 8.0 Hz, 2H), 6.92 (d, J = 8.2 Hz, 2H), 5.12- |
| | yl)isoxazol-5-yl)-3-methylbutanoyl)- | | 5.02 (m, 1H), 4.93-4.85 (m, 1H), 4.37 (t, J = |
| | 4-hydroxy-N-((S)-1- | | 7.9 Hz, 1H), 4.34-4.24 (m, 2H), 4.13 (t, J = |
| | phenylethyl)pyrrolidine-2- | | 7.7 Hz, 1H), 3.90 (d, J = 3.8 Hz, 1H), 3.84 (d, |
| | carboxamide | | J = 9.8 Hz, 1H), 3.75 (q, J = 9.3 Hz, 3H), 3.48 (s, |
| | | | 2H), 2.63 (s, 1H), 2.13 (s, 1H), 2.36 (s, 1H), |
| | | | 2.00 (d, J = 8.6 Hz, 1H), 1.80-1.73 (m, 1H), |
| | | | 1.49-1.33 (m, 5H), 1.21 (d, J = 3.2 Hz, 2H), |
| | | | 1.01 (d, J = 6.5 Hz, 3H), 0.84 (t, J = 7.1 Hz, |
| | | | 3H). |
| 64 | (2S,4R)-1-((R)-2-(3-(2-(6-(5- | 931.3 | ¹H NMR (400 MHz, DMSO-d6) δ 8.99 (s, |
| | (bicyclo[1.1.1]pentan-1-yl)-3-(2- | | 1H), 8.79 (d, J = 2.5 Hz, 2H), 8.44 (d, J = |
| | hydroxyphenyl)-5H-pyrrolo[3,2- | | 7.7 Hz, 1H), 8.36 (s, 1H), 8.05 (s, 1H), |
| | c]pyridazin-6-yl)-2- | | 7.49-7.41 (m, 2H), 7.37 (dd, J = 8.5, 2.9 |
| | azaspiro[3.3]heptan-2-yl)pyrimidin- | | Hz, 3H), 7.10 (d, J = 15.7 Hz, 1H), 7.01 (d, |
| | 5-yl)isoxazol-5-yl)-3- | | J = 7.7 Hz, 2H), 6.86 (d, J = 45.0 Hz, 1H), |
| | methylbutanoyl)-4-hydroxy-N-((S)- | | 5.10 (s, 1H), 4.93 (t, J = 7.3 Hz, 1H), 4.38 |
| | 1-(4-(4-methylthiazol-5- | | (t, J = 7.9 Hz, 1H), 4.28 (d, J = 13.0 Hz, |
| | yl)phenyl)ethyl)pyrrolidine-2- | | 3H), 4.17 (s, 2H), 3.87 (dd, J = 20.9, 8.8 |
| | carboxamide | | Hz, 2H), 3.75 (d, J = 6.7 Hz, 1H), 3.49 (d, |
| | | | J = 10.8 Hz, 1H), 2.86 (t, J = 10.4 Hz, 2H), |
| | | | 2.73 (s, 1H), 2.65 (s, 6H), 2.62-2.55 (m, |
| | | | 2H), 2.46 (s, 3H), 2.33 (s, 2H), 2.03 (t, J = |
| | | | 9.9 Hz, 1H), 1.84-1.71 (m, 1H), 1.43 (dd, |
| | | | J = 39.4, 7.0 Hz, 3H), , 1.00 (d, J = 6.3 Hz, |
| | | | 3H), 0.83 (d, J = 6.8 Hz, 3H) |
| 62 | (2S,4R)-1-((R)-2-(3-(2-(6-(5- | 919.35 | ¹H NMR (400 MHz, DMSO-d6) δ 14.18 (s, |
| | cyclobutyl-3-(2-hydroxyphenyl)-5H- | | 1H), 8.99 (s, 1H), 8.80 (s, 2H), 8.47-8.40 |
| | pyrrolo[3,2-c]pyridazin-6-yl)-2- | | (m, 2H), 8.21-8.14 (m, 1H), 7.48-7.28 |
| | azaspiro[3.3]heptan-2-yl)pyrimidin- | | (m, 5H), 7.04-6.95 (m, 2H), 6.91 (d, J = |
| | 5-yl)isoxazol-5-yl)-3- | | 4.0 Hz, 2H), 5.11 (d, J = 3.6 Hz, 1H), 5.00- |
| | methylbutanoyl)-4-hydroxy-N-((S)- | | 4.87 (m, 2H), 4.38 (t, J = 7.9 Hz, 1H), |
| | 1-(4-(4-methylthiazol-5- | | 4.31 (s, 3H), 4.11 (s, 2H), 3.88-3.71 (m, |
| | yl)phenyl)ethyl)pyrrolidine-2- | | 3H), 3.50 (d, J = 10.6 Hz, 1H), 3.32 (d, J = |
| | carboxamide | | 10.6 Hz, 1H), 2.87 (m, J = 21.5, 11.7, 5.7 |
| | | | Hz, 4H), 2.56 (s, 2H), 2.45 (d, J = 6.6 Hz, |
| | | | 3H), 2.11-1.99 (m, 2H), 1.94-1.74 (m, |
| | | | 2H), 1.74-1.65 (m, 2H), 1.48 (d, J = 6.9 |
| | | | Hz, 3H), 1.39 (d, J = 7.0 Hz, 3H), 0.84 (t, |
| | | | J = 7.3 Hz, 3H). |
| 69 | (2S,4R)-1-((R)-2-(3-(2-((R)-3-(3-(3- | 913.35 | ¹H NMR (400 MHz, DMSO-d6) δ 15.89 (s, |
| | chloro-2-hydroxyphenyl)-5- | | 1H), 9.01-8.96 (m, 1H), 8.88-8.84 (m, |
| | cyclopropyl-5H-pyrrolo[3,2- | | 2H), 8.49-8.41 (m, 2H), 8.25-8.18 (m, |
| | c]pyridazin-6-yl)pyrrolidin-1- | | 1H), 7.54-7.34 (m, 5H), 7.05-6.91 (m, |
| | yl)pyrimidin-5-yl)isoxazol-5-yl)-3- | | 3H), 5.12 (d, J = 3.6 Hz, 1H), 5.00-4.87 |
| | methylbutanoyl)-4-hydroxy-N-((S)- | | (m, 1H), 4.43-4.25 (m, 3H), 4.21-4.09 |
| | 1-(4-(4-methylthiazol-5- | | (m, 1H), 3.95-3.81 (m, 2H), 3.80-3.58 |
| | yl)phenyl)ethyl)pyrrolidine-2- | | (m, 3H), 3.54-3.45 (m, 2H), 2.67-2.59 |
| | carboxamide | | (m, 1H), 2.48-2.42 (m, 3H), 2.39-2.28 |
| | | | (m, 2H), 2.09-1.99 (m, 1H), 1.85-1.73 |
| | | | (m, 1H), 1.49 (d, J = 7.0 Hz, 1H), 1.45- |
| | | | 1.32 (m, 4H), 1.26-1.19 (m, 2H), 1.05- |
| | | | 0.98 (m, 3H), 0.89-0.81 (m, 3H). |
| 70 | (2S,4R)-1-((R)-2-(3-(2-((R)-3-(3-(3- | 931.45 | ¹H NMR (400 MHz, DMSO-d6) δ 15.74 (s, |
| | chloro-5-fluoro-2-hydroxyphenyl)- | | 1H), 8.99 (d, J = 2.0 Hz, 1H), 8.85 (d, J = |
| | 5-cyclopropyl-5H-pyrrolo[3,2- | | 5.7 Hz, 2H), 8.52 (s, 1H), 8.44 (d, J = 7.7 |
| | c]pyridazin-6-yl)pyrrolidin-1- | | Hz, 1H), 8.22 (dd, J = 10.4, 3.0 Hz, 1H), |
| | yl)pyrimidin-5-yl)isoxazol-5-yl)-3- | | 7.52 (dd, J = 8.1, 3.0 Hz, 1H), 7.47-7.42 |
| | methylbutanoyl)-4-hydroxy-N-((S)- | | (m, 2H), 7.40-7.33 (m, 2H), 6.97 (d, J = |
| | 1-(4-(4-methylthiazol-5- | | 6.0 Hz, 1H), 6.93 (s, 1H), 5.08 (dd, J = |
| | yl)phenyl)ethyl)pyrrolidine-2- | | 30.2, 3.2 Hz, 1H), 4.98-4.65 (m, 1H), |
| | carboxamide | | 4.39 (t, J = 7.9 Hz, 1H), 4.30 (dd, J = 11.1, |
| | | | 7.2 Hz, 2H), 4.15 (p, J = 7.7 Hz, 1H), 4.01- |
| | | | 3.82 (m, 2H), 3.81-3.57 (m, 3H), 3.55- |
| | | | 3.41 (m, 2H), 2.74-2.55 (m, 1H), 2.45 (d, |
| | | | J = 7.1 Hz, 3H), 2.41-2.24 (m, 2H), 2.11- |
| | | | 1.92 (m, 1H), 1.86-1.69 (m, 1H), 1.55- |
| | | | 1.29 (m, 5H), 1.25-1.20 (m, 2H), 1.02 |
| | | | (dd, J = 6.7, 3.9 Hz, 3H), 0.85 (t, J = 7.2 |
| | | | Hz, 3H). |
| 73 | (2S,4R)-1-((R)-2-(3-(2-((S)-3-(3-(3- | 931.3 | ¹H NMR (400 MHz, DMSO-d6) δ 15.73 (s, |
| | chloro-5-fluoro-2-hydroxyphenyl)- | | 1H), 8.99 (s, 1H), 8.85 (d, J = 4.9 Hz, 2H), |
| | 5-cyclopropyl-5H-pyrrolo[3,2- | | 8.52 (s, 1H), 8.44 (d, J = 7.7 Hz, 1H), 8.21 |
| | c]pyridazin-6-yl)pyrrolidin-1- | | (dd, J = 10.3, 3.1 Hz, 1H), 7.52 (dd, J = |
| | yl)pyrimidin-5-yl)isoxazol-5-yl)-3- | | 8.0, 3.0 Hz, 1H), 7.48-7.42 (m, 2H), 7.41- |
| | methylbutanoyl)-4-hydroxy-N-((S)- | | 7.33 (m, 2H), 7.01-6.77 (m, 2H), 5.01- |
| | 1-(4-(4-methylthiazol-5- | | 4.68 (m, 1H), 4.39 (t, J = 7.9 Hz, 1H), 4.29 |
| | yl)phenyl)ethyl)pyrrolidine-2- | | (dd, J = 11.3, 7.3 Hz, 2H), 4.15 (p, J = 7.8 |
| | carboxamide | | Hz, 1H), 3.95-3.82 (m, 2H), 3.75 (dtd, J = |
| | | | 12.4, 7.9, 3.4 Hz, 3H), 3.52 (s, 2H), 2.70- |
| | | | 2.58 (m, 1H), 2.45 (d, J = 6.7 Hz, 3H), 2.39- |
| | | | 2.28 (m, 2H), 2.12-1.87 (m, 2H), 1.80 |
| | | | (ddd, J = 12.8, 8.1, 4.7 Hz, 1H), 1.53- |
| | | | 1.31 (m, 5H), 1.27-1.15 (m, 2H), 1.02 (d, |
| | | | J = 6.3 Hz, 3H), 0.85 (t, J = 7.3 Hz, 3H). |
| 95 | (2S,4R)-1-((R)-2-(3-(2-((S)-3-(5- | 905.4 | ¹H NMR (400 MHz, DMSO-d6) δ 13.91 (s, |
| | (bicyclo[1.1.1]pentan-1-yl)-3-(2- | | 1H), 9.01-8.96 (m, 1H), 8.90-8.80 (m, |
| | hydroxyphenyl)-5H-pyrrolo[3,2- | | 2H), 8.44 (d, J = 7.7 Hz, 1H), 8.36 (s, 1H), |
| | c]pyridazin-6-yl)pyrrolidin-1- | | 8.15-8.08 (m, 1H), 7.48-7.41 (m, 2H), |
| | yl)pyrimidin-5-yl)isoxazol-5-yl)-3- | | 7.41-7.28 (m, 3H), 7.03-6.95 (m, 2H), |
| | methylbutanoyl)-4-hydroxy-N-((S)- | | 6.94-6.78 (m, 2H), 5.14-5.09 (m, 1H), |
| | 1-(4-(4-methylthiazol-5- | | 5.06-4.69 (m, 1H), 4.44-4.35 (m, 1H), |
| | yl)phenyl)ethyl)pyrrolidine-2- | | 4.31 (s, 1H), 4.18-4.06 (m, 2H), 3.94- |
| | carboxamide | | 3.59 (m, 5H), 3.55-3.47 (m, 1H), 2.79- |
| | | | 2.73 (m, 7H), 2.61-2.51 (m, 1H), 2.48- |
| | | | 2.42 (m, 3H), 2.39-2.21 (m, 2H), 2.09- |
| | | | 1.99 (m, 1H), 1.86-1.75 (m, 1H), 1.54- |
| | | | 1.35 (m, 3H), 1.06-0.99 (m, 3H), 0.89- |
| | | | 0.81 (m, 3H). |
| 94 | (2S,4R)-1-((R)-2-(3-(2-((R)-3-(5- | 905.35 | ¹H NMR (400 MHz, DMSO-d6) δ 13.90 (s, |
| | (bicyclo[1.1.1]pentan-1-yl)-3-(2- | | 1H), 8.99 (s, 1H), 8.86 (s, 2H), 8.44 (d, J = |
| | hydroxyphenyl)-5H-pyrrolo[3,2- | | 7.7 Hz, 1H), 8.36 (s, 1H), 8.12 (d, 1H), |
| | c]pyridazin-6-yl)pyrrolidin-1- | | 7.48-7.29 (m, 5H), 7.03-6.95 (m, 2H), |
| | yl)pyrimidin-5-yl)isoxazol-5-yl)-3- | | 6.95-6.79 (m, 2H), 5.08 (dd, J = 29.9, 3.2 |
| | methylbutanoyl)-4-hydroxy-N-((S)- | | Hz, 1H), 5.00-4.69 (m, 1H), 4.39 (t, J = |
| | 1-(4-(4-methylthiazol-5- | | 7.9 Hz, 1H), 4.30 (s, 1H), 4.15-4.07 (m, |
| | yl)phenyl)ethyl)pyrrolidine-2- | | 2H), 3.94-3.58 (m, 5H), 3.51 (d, J = 10.5 |
| | carboxamide | | Hz, 1H), 2.78-2.74 (m, 7H), 2.70-2.52 |
| | | | (m, 1H), 2.45 (d, J = 7.3 Hz, 3H), 2.35- |
| | | | 2.25 (m, 2H), 2.09-1.97 (m, 1H), 1.85- |
| | | | 1.74 (m, 1H), 1.44 (dd, J = 40.7, 7.0 Hz, |
| | | | 3H), 1.02 (t, J = 5.3 Hz, 3H), 0.84 (t, J = |
| | | | 6.8 Hz, 3H). |
| 102 | (2S,4R)-1-((R)-2-(3-(2-((R)-3-(5- | 893.2 | ¹H NMR (300 MHz, DMSO-d6) δ 14.01 (s, |
| | cyclobutyl-3-(2-hydroxyphenyl)-5H- | | 1H), 8.99 (d, J = 1.5 Hz, 1H), 8.86 (d, J = |
| | pyrrolo[3,2-c]pyridazin-6- | | 3.6 Hz, 2H), 8.60-8.32 (m, 2H), 8.19 (d, J = |
| | yl)pyrrolidin-1-yl)pyrimidin-5- | | 7.9 Hz, 1H), 7.51-7.35 (m, 5H), 7.11- |
| | yl)isoxazol-5-yl)-3-methylbutanoyl)- | | 6.86 (m, 4H), 5.24 (q, J = 8.9 Hz, 1H), 5.12 |
| | 4-hydroxy-N-((S)-1-(4-(4- | | (s, 1H), 4.94 (t, J = 7.3 Hz, 1H), 4.40 (s, |
| | methylthiazol-5- | | 1H), 4.31 (s, 1H), 4.20 (dd, J = 11.1, 7.1 |
| | yl)phenyl)ethyl)pyrrolidine-2- | | Hz, 1H), 4.01 (t, J = 7.2 Hz, 1H), 3.88- |
| | carboxamide | | 3.79 (m, 2H), 3.74 (s, 3H), 3.51 (d, J = |
| | | | 10.0 Hz, 1H), 2.99 (d, J = 10.5 Hz, 2H), |
| | | | 2.57 (d, J = 8.2 Hz, 2H), 2.46 (d, J = 5.1 |
| | | | Hz, 3H), 2.29 (td, J = 15.4, 13.6, 8.1 Hz, |
| | | | 3H), 2.18-1.98 (m, 2H), 1.98-1.74 (m, |
| | | | 2H), 1.39 (d, J = 7.0 Hz, 3H), 1.03 (s, 3H), |
| | | | 0.84 (d, J = 6.6 Hz, 3H). |
| 103 | (2S,4R)-4-hydroxy-1-((R)-2-(3-(2- | 893.35 | ¹H NMR (300 MHz, DMSO-d6) δ 14.24 (s, |
| | ((R)-3-(3-(2-hydroxyphenyl)-5-(1- | | 1H), 8.99 (s, 1H), 8.87 (s, 2H), 8.49-8.38 |
| | methylcyclopropyl)-5H-pyrrolo[3,2- | | (t, J = 11.2 Hz, 2H), 8.24-8.15 (m, 1H), |
| | c]pyridazin-6-yl)pyrrolidin-1- | | 7.49-7.28 (m, 5H), 7.06-6.90 (m, 4H), |
| | yl)pyrimidin-5-yl)isoxazol-5-yl)-3- | | 5.17-5.01 (t, J = 3.6 Hz, 1H), 4.99-4.88 |
| | methylbutanoyl)-N-((S)-1-(4-(4- | | (m, 1H), 4.45-4.34 (t, J = 7.9 Hz, 1H), |
| | methylthiazol-5- | | 4.33-4.23(m, 2H), 4.19-4.09 (t, J = 8.8 |
| | yl)phenyl)ethyl)pyrrolidine-2- | | Hz, 1H), 4.01-3.90 (m, 1H), 3.89-3.66 |
| | carboxamide | | (m, 4H), 3.57-3.46 (t, J = 10.7 Hz, 1H), |
| | | | 2.70-2.57 (m, 1H), 2.48-2.20 (m, 6H), |
| | | | 2.11-1.98 (m, 1H), 1.86-1.72 (m, 1H), |
| | | | 1.61(s, 3H), 1.52-1.30 (m, 5H), 1.08-0.95 |
| | | | (d, J = 6.5 Hz, 3H), 0.90-0.77 (t, J = 6.6 |
| | | | Hz, 4H). |
| 104 | (2S,4R)-4-hydroxy-1-((R)-2-(3-(2- | 893.35 | ¹H NMR (300 MHz, DMSO-d6) δ 14.22 (s, |
| | ((S)-3-(3-(2-hydroxyphenyl)-5-(1- | | 1H), 8.99 (s, 1H), 8.90-8.84 (m, 2H), |
| | methylcyclopropyl)-5H-pyrrolo[3,2- | | 8.47-8.40 (t, J = 9.5 Hz, 2H), 8.23-8.17 |
| | c]pyridazin-6-yl)pyrrolidin-1- | | (m, J = 7.9 Hz, 1H), 7.48-7.28 (m, 5H), |
| | yl)pyrimidin-5-yl)isoxazol-5-yl)-3- | | 7.03-6.90 (m, 4H), 5.17-4.88(m, 2H), |
| | methylbutanoyl)-N-((S)-1-(4-(4- | | 4.43-4.23 (m, 4H), 3.88-3.63 (m, 2H), |
| | methylthiazol-5- | | 3.58-3.43 (m, 4H), 2.75-2.53(m, 1H), |
| | yl)phenyl)ethyl)pyrrolidine-2- | | 2.43-2.41 (d, J = 4.3 Hz, 3H), 2.40-2.24 |
| | carboxamide | | (m, 3H), 2.11-1.91(m,1H), 1.88-1.69 |
| | | | (m, 1H), 1.60 (s, 3H), 1.52-1.30 (m, 5H), |
| | | | 1.06-0.97 (m, 3H), 0.89-0.80 (m, 4H). |
| 106 | (2S,4R)-1-((R)-2-(3-(2-((R)-3-(5- | 897.15 | ¹H NMR (400 MHz, DMSO-d6) δ 15.00 (s, |
| | cyclopropyl-3-(3-fluoro-2- | | 1H), 8.98 (s, 1H), 8.85 (d, J = 1.4 Hz, 2H), |
| | hydroxyphenyl)-5H-pyrrolo[3,2- | | 8.41 (d, J = 5.3 Hz, 2H), 8.02 (d, J = 8.2 |
| | c]pyridazin-6-yl)pyrrolidin-1- | | Hz, 1H), 7.48-7.41 (m, 2H), 7.38 (d, J = |
| | yl)pyrimidin-5-yl)isoxazol-5-yl)-3- | | 8.3 Hz, 2H), 7.34-7.25 (m, 1H), 7.02- |
| | methylbutanoyl)-4-hydroxy-N-((S)- | | 6.90 (m, 3H), 5.10 (d, J = 3.7 Hz, 1H), 4.93 |
| | 1-(4-(4-methylthiazol-5- | | (t, J = 7.2 Hz, 1H), 4.39 (t, J = 7.9 Hz, 1H), |
| | yl)phenyl)ethyl)pyrrolidine-2- | | 4.29 (t, J = 9.1 Hz, 2H), 4.19-4.10 (m, |
| | carboxamide | | 1H), 3.93-3.82 (m, 2H), 3.80-3.71 (m, |
| | | | 3H), 3.50 (d, J = 10.9 Hz, 2H), 2.63 (d, J = |
| | | | 8.9 Hz, 1H), 2.45 (d, J = 6.7 Hz, 3H), 2.36 |
| | | | (s, 2H), 2.04 (dd, J = 18.5, 7.1 Hz, 1H), |
| | | | 1.80 (dd, J = 12.8, 8.1, 4.8 Hz, 1H), 1.49 |
| | | | (d, J = 6.9 Hz, 3H), 1.37 (dd, J = 17.7, 7.0 |
| | | | Hz, 2H), 1.31-1.22 (m, 2H), 1.01 (d, J = |
| | | | 6.5 Hz, 3H), 0.85 (t, J = 7.2 Hz, 3H). |
| 108 | (2S,4R)-1-((R)-2-(3-(2-((S)-3-(5- | 915.2 | ¹H NMR (400 MHz, DMSO-d6) δ 14.83 (s, |
| | cyclopropyl-3-(3,5-difluoro-2- | | 1H), 8.98 (s, 1H), 8.88-8.83 (m, 2H), |
| | hydroxyphenyl)-5H-pyrrolo[3,2- | | 8.56 (s, 1H), 8.42 (d, J = 7.7 Hz, 1H), 8.01 |
| | c]pyridazin-6-yl)pyrrolidin-1- | | (d, J = 10.3 Hz, 1H), 7.48-7.41 (m, 2H), |
| | yl)pyrimidin-5-yl)isoxazol-5-yl)-3- | | 7.41-7.27 (m, 3H), 6.97-6.78 (m, 2H), |
| | methylbutanoyl)-4-hydroxy-N-((S)- | | 5.16-4.69 (m, 2H), 4.44-4.35 (m, 1H), |
| | 1-(4-(4-methylthiazol-5- | | 4.34-4.25 (m, 2H), 4.18-4.08 (m, 1H), |
| | yl)phenyl)ethyl)pyrrolidine-2- | | 3.94-3.81 (m, 2H), 3.81-3.64 (m, 3H), |
| | carboxamide | | 3.54-3.44 (m, 2H), 2.66-2.59 (m, 1H), |
| | | | 2.48-2.42 (m, 3H), 2.40-2.27 (m, 2H), |
| | | | 2.06-2.01 (m, 1H), 1.86-1.75 (m, 1H), |
| | | | 1.54-1.31 (m, 3H), 1.28-1.22 (m, 2H), |
| | | | 1.22-1.16 (m, 2H), 1.05-0.98 (m, 3H), |
| | | | 0.89-0.81 (m, 3H). |
| 109 | (2S,4R)-1-((R)-2-(3-(2-((S)-3-(5- | 897.15 | ¹H NMR (400 MHz, DMSO-d6) δ 15.00 (s, |
| | cyclopropyl-3-(3-fluoro-2- | | 1H), 8.98 (s, 1H), 8.85 (s, 2H), 8.41 (d, J = |
| | hydroxyphenyl)-5H-pyrrolo[3,2- | | 5.3 Hz, 2H), 8.02 (d, J = 8.1 Hz, 1H), 7.44 |
| | c]pyridazin-6-yl)pyrrolidin-1- | | (d, J = 8.2 Hz, 2H), 7.38 (d, J = 8.2 Hz, |
| | yl)pyrimidin-5-yl)isoxazol-5-yl)-3- | | 2H), 7.34-7.25 (m, 1H), 7.02-6.90 (m, |
| | methylbutanoyl)-4-hydroxy-N-((S)- | | 3H), 5.10 (d, J = 3.7 Hz, 1H), 4.97-4.89 |
| | 1-(4-(4-methylthiazol-5- | | (m, 1H), 4.39 (t, J = 7.9 Hz, 1H), 4.28 (d, |
| | yl)phenyl)ethyl)pyrrolidine-2- | | J = 11.6 Hz, 2H), 4.19-4.10 (m, 1H), 3.98- |
| | carboxamide | | 3.81 (m, 2H), 3.75 (d, J = 9.7 Hz, 3H), |
| | | | 3.53-3.46 (m, 2H), 2.64 (s, 1H), 2.45 (d, |
| | | | J = 7.2 Hz, 3H), 2.36 (s, 1H), 2.14-1.95 |
| | | | (m, 2H), 1.79 (dd, J = 12.8, 5.0 Hz, 1H), |
| | | | 1.49 (d, J = 7.0 Hz, 3H), 1.42-1.25 (m, |
| | | | 2H), 1.22 (d, J = 10.7 Hz, 2H), 1.01 (d, J = |
| | | | 6.6 Hz, 3H), 0.84 (d, J = 6.9 Hz, 3H). |
| 111 | (2S,4R)-1-((R)-2-(3-(2-((R)-3-(5- | 915.2 | ¹H NMR (400 MHz, DMSO-d6) δ 14.83 (s, |
| | cyclopropyl-3-(3,5-difluoro-2- | | 1H), 8.98 (d, J = 2.0 Hz, 1H), 8.86 (s, 2H), |
| | hydroxyphenyl)-5H-pyrrolo[3,2- | | 8.49 (s, 1H), 8.43 (d, J = 7.7 Hz, 1H), 8.03 |
| | c]pyridazin-6-yl)pyrrolidin-1- | | (dt, J = 10.6, 2.2 Hz, 1H), 7.48-7.41 (m, |
| | yl)pyrimidin-5-yl)isoxazol-5-yl)-3- | | 2H), 7.41-7.32 (m, 3H), 6.99-6.91 (m, |
| | methylbutanoyl)-4-hydroxy-N-((S)- | | 2H), 5.15-4.65 (m, 2H), 4.39 (t, J = 7.9 |
| | 1-(4-(4-methylthiazol-5- | | Hz, 1H), 4.34-4.25 (m, 2H), 4.21-4.09 |
| | yl)phenyl)ethyl)pyrrolidine-2- | | (m, 1H), 3.95-3.81 (m, 2H), 3.81-3.64 |
| | carboxamide | | (m, 3H), 3.54-3.44 (m, 2H), 2.67-2.60 |
| | | | (m, 1H), 2.48-2.42 (m, 3H), 2.41-1.92 |
| | | | (m, 3H), 1.85-1.75 (m, 1H), 1.52-1.31 |
| | | | (m, 5H), 1.05-0.98 (m, 2H), 0.89-0.81 |
| | | | (m, 6H). |
| 112 | (2S,4R)-1-((R)-2-(3-(2-((S)-3-(5- | 893.2 | ¹H NMR (400 MHz, Chloroform-d) δ 8.71 |
| | cyclobutyl-3-(2-hydroxyphenyl)-5H- | | (d, J = 18.6 Hz, 3H), 8.17 (s, 1H), 7.74 (d, |
| | pyrrolo[3,2-c]pyridazin-6- | | J = 7.8 Hz, 1H), 7.16 (d, J = 8.2 Hz, 1H), |
| | yl)pyrrolidin-1-yl)pyrimidin-5- | | 6.97 (t, J = 7.6 Hz, 1H), 6.78 (s, 1H), 6.56 |
| | yl)isoxazol-5-yl)-3-methylbutanoyl)- | | (s, 1H), 5.08 (dd, J = 16.6, 9.2 Hz, 2H), |
| | 4-hydroxy-N-((S)-1-(4-(4- | | 4.68 (d, J = 8.8 Hz, 2H), 4.22 (t, J = 9.3 |
| | methylthiazol-5- | | Hz, 1H), 3.79 (ddd, J = 45.9, 34.0, 23.7 |
| | yl)phenyl)ethyl)pyrrolidine-2- | | Hz, 8H), 2.99 (q, J = 10.3 Hz, 2H), 2.72 (s, |
| | carboxamide | | 2H), 2.53 (s, 6H), 2.32 (s, 1H), 2.19 (d, J = |
| | | | 10.4 Hz, 1H), 2.10-2.02 (m, 1H), 2.00 (s, |
| | | | 2H), 1.51 (d, J = 7.0 Hz, 3H), 1.10 (d, J = |
| | | | 6.6 Hz, 3H). |
| 114 | (2S,4R)-1-((R)-2-(3-(2-((R)-3-(5- | 893.2 | ¹H NMR (400 MHz, DMSO-d6) δ 14.46 (s, |
| | (cyclopropylmethyl)-3-(2- | | 1H), 8.99 (s, 1H), 8.86 (s, 2H), 8.65 (s, |
| | hydroxyphenyl)-5H-pyrrolo[3,2- | | 1H), 8.44 (d, J = 7.7 Hz, 1H), 8.19 (d, J = |
| | c]pyridazin-6-yl)pyrrolidin-1- | | 7.8 Hz, 1H), 7.48-7.41 (m, 2H), 7.38- |
| | yl)pyrimidin-5-yl)isoxazol-5-yl)-3- | | 7.28 (m, 3H), 7.03-6.91 (m, 4H), 5.11 (d, |
| | methylbutanoyl)-4-hydroxy-N-((S)- | | J = 3.6 Hz, 1H), 4.95 (dt, J = 14.3, 7.2 Hz, |
| | 1-(4-(4-methylthiazol-5- | | 1H), 4.39 (t, J = 7.7 Hz, 3H), 4.31 (s, 2H), |
| | yl)phenyl)ethyl)pyrrolidine-2- | | 4.01-3.81 (m, 3H), 3.75 (dt, J = 12.7, 7.0 |
| | carboxamide | | Hz, 3H), 3.74-3.58 (m, 1H), 2.59 (d, J = |
| | | | 6.4 Hz, 1H), 2.45 (d, J = 6.1 Hz, 3H), 2.30 |
| | | | (s, 2H), 2.06 (d, J = 16.0 Hz, 1H), 1.99- |
| | | | 1.80 (m, 1H), 1.39 (d, J = 7.0 Hz, 3H), 1.34- |
| | | | 1.21 (m, 1H), 1.01 (d, J = 6.5 Hz, 3H), |
| | | | 0.85 (t, J = 7.2 Hz, 3H), 0.53 (d, J = 7.8 |
| | | | Hz, 4H) |
| 115 | (2S,4R)-1-((R)-2-(3-(2-((S)-3-(5- | 893.4 | ¹H NMR (400 MHz, DMSO-d6) δ 14.46 (s, |
| | (cyclopropylmethyl)-3-(2- | | 1H), 8.99 (s, 1H), 8.86 (s, 2H), 8.65 (s, |
| | hydroxyphenyl)-5H-pyrrolo[3,2- | | 1H), 8.44 (d, J = 7.7 Hz, 1H), 8.25-8.16 |
| | c]pyridazin-6-yl)pyrrolidin-1- | | (m, 1H), 7.45 (d, J = 8.1 Hz, 2H), 7.41- |
| | yl)pyrimidin-5-yl)isoxazol-5-yl)-3- | | 7.32 (m, 3H), 6.98 (d, J = 7.9 Hz, 3H), |
| | methylbutanoyl)-4-hydroxy-N-((S)- | | 6.93 (s, 1H), 5.12 (s, 1H), 4.94 (t, J = 6.8 |
| | 1-(4-(4-methylthiazol-5- | | Hz, 1H), 4.39 (t, J = 7.7 Hz, 2H), 4.26 (dd, |
| | yl)phenyl)ethyl)pyrrolidine-2- | | J = 21.3, 12.7 Hz, 2H), 3.99 (t, J = 7.5 Hz, |
| | carboxamide | | 1H), 3.82 (s, 1H), 3.75 (dt, J = 13.3, 7.5 |
| | | | Hz, 2H), 3.62 (s, 1H),3.30 (s, 2H), 2.59 (s, |
| | | | 1H), 2.45 (d, J = 6.5 Hz, 3H), 2.34 (d, J = |
| | | | 8.1 Hz, 2H), 2.06 (d, J = 15.4 Hz, 1H), |
| | | | 1.81 (s, 1H), 1.49 (d, J = 6.9 Hz, 1H), 1.52 |
| | | | (s, 1H), 1.45-1.36 (m, 2H), 1.34-1.28 |
| | | | (m, 1H), 1.01 (d, J = 6.5 Hz, 3H), 0.85 (t, |
| | | | J = 7.1 Hz, 3H), 0.53 (d, J = 7.7 Hz, 4H). |

### Preparation of methyl 2-(3-(6-fluoro-5-methylpyridin-3-yl)isoxazol-5-yl)-3-methylbutanoate (I-11)

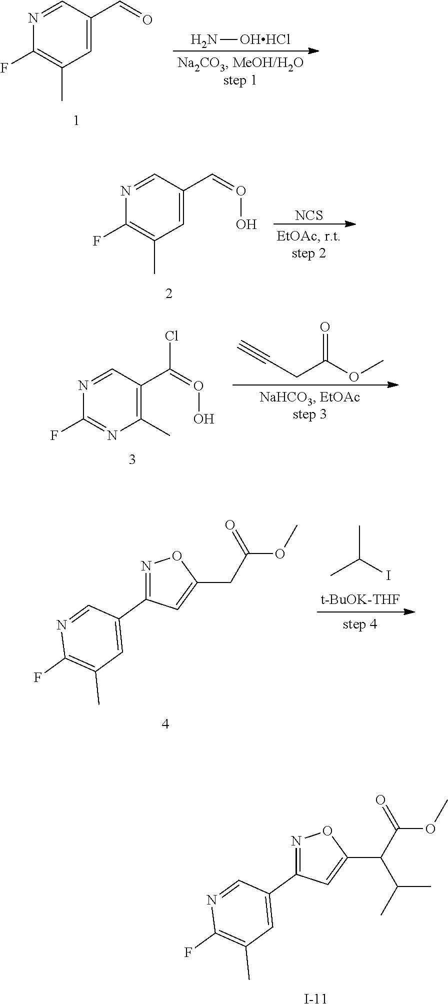

### Step 1: Preparation of (Z)—N-[(6-fluoro-5-methylpyridin-3-yl) methylidene]hydroxylamine (Intermediate 2)

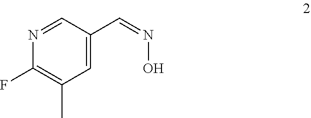

To a solution of 6-chloro-5-methylpyridine-3-carbaldehyde (2.0 g, 12.855 mmol, 1 equiv), hydroxylamine hydrochloride (1.79 g, 25.710 mmol, 2 equiv) in methanol (15 mL) were added Na₂CO₃ (4.09 g, 38.565 mmol, 3 equiv) and water (15 mL). The resulting solution was stirred at 25 degrees C. for 2h. Desired product could be detected by LCMS. The mixture was diluted with ethyl acetate (500 ml) and washed with water (500 ml×3). The organic layer was dried over anhydrous sodium sulfate, filtered and concentrated to give intermediate 2 (2.1 g, crude) as white solid. LCMS (ESI) m/z: [M+H]⁺=155

### Step 2: Preparation of (E)-6-fluoro-N-hydroxy-2-methylpyridine-3-carbonimidoyl chloride (Intermediate 3)

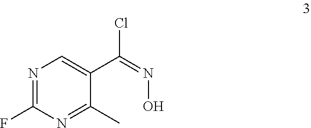

To a solution of intermediate 2 (2.1 g, 14.272 mmol, 1 equiv) in ethyl acetate (20 mL) was added NCS (2.86 g, 21.408 mmol, 1.5 equiv). The resulting solution was stirred at 25 degrees C. overnight. The mixture was diluted with ethyl acetate (500 mL) and washed with water (300 mL×3). The organic layer was dried over anhydrous sodium sulfate, filtered and concentrated to give intermediate 3 (3.6 g, crude) as a white solid. LCMS (ESI) m/z: [M+H]⁺=189.

### Step 3: Preparation of methyl 2-[3-(6-fluoro-5-methylpyridin-3-yl)-1,2-oxazol-5-yl]acetate (Intermediate 4)

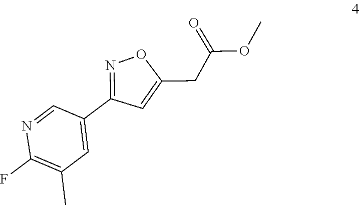 [SMILES: COC(=O)Cc1cc(-c2cnc(F)c(C)c2)no1]

To a solution of intermediate 3 (3.6 g, 19.089 mmol, 1 equiv) in ethyl acetate (14 mL) were added methyl but-3-ynoate (3.75 g, 38.178 mmol, 2 equiv) and NaHCO₃ (4.81 g, 57.267 mmol, 3 equiv) at 0 degrees. The resulting solution was stirred at 25 degrees C. overnight. The mixture was diluted with ethyl acetate (500 mL) and washed with water (500 mL×3). The organic layer was dried over anhydrous sodium sulfate, filtered and concentrated to give a crude product. The crude product was purified by reverse flash chromatography with the following conditions: column, C18 silica gel; mobile phase, acetonitrile in water, 0% to 100% gradient in 30 min to give intermediate 4 (2.5 g, 52.34%) as a white solid. LCMS (ESI) m/z: [M+H]⁺=251.

### Step 4: Preparation of methyl 2-[3-(6-fluoro-5-methylpyridin-3-yl)-1,2-oxazol-5-yl]-3-methylbutanoate (I-11)

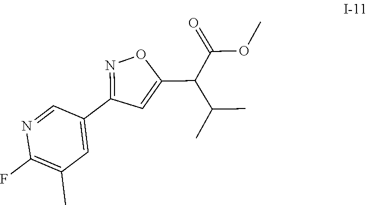 [SMILES: COC(=O)C(c1cc(-c2cnc(F)c(C)c2)no1)C(C)C]

To a solution of intermediate 4 (500 mg, 1.998 mmol, 1 equiv), 2-iodopropane (679.35 mg, 3.996 mmol, 2 equiv) in THF (5 mL) was added Cs₂CO₃ (1302.08 mg, 3.996 mmol, 2 equiv). The resulting solution was stirred at 60 degrees C. overnight. The mixture was diluted with ethyl acetate (200 mL) and washed with water (200 mL×3). The organic layer was dried over anhydrous sodium sulfate, filtered and concentrated to give a crude product. The crude product was purified by flash C18 chromatography, elution gradient 0 to 60% acetonitrile in water to give I-11 (288 mg, 49.31%) as a white solid. LCMS (ESI) m/z: [M+H]⁺=293.

The following intermediates in Table 9 were prepared in a similar manner as described in the preparation of intermediate I-11 starting with the appropriate aldehyde.

TABLE 9

| | Inter- | | LCMS |
|---|---|---|---|
| | mediate | | (ESI) |
| Structure | No. | Name | m/z |
| 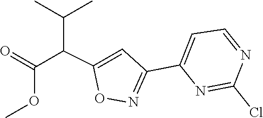 [SMILES: COC(=O)C(c1cc(-c2ccnc(Cl)n2)no1)C(C)C] | I-12 | methyl 2-(3-(2-chloropyrimidin- 4-yl)isoxazol-5-yl)-3- methylbutanoate | 296 |
| 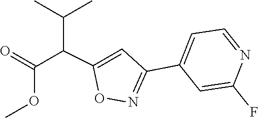 [SMILES: COC(=O)C(c1cc(-c2ccnc(F)c2)no1)C(C)C] | I-13 | methyl 2-(3-(2-fluoropyridin-4- yl)isoxazol-5-yl)-3- methylbutanoate | 279 |
| 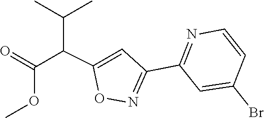 [SMILES: COC(=O)C(c1cc(-c2cc(Br)ccn2)no1)C(C)C] | I-14 | 2-[3-(4-bromopyridin-2-yl)-1,2- oxazol-5-yl]-3-methylbutanoate | 339 |

### Preparation of (2S,4R)-1-[(2R)-2-(3-{6-[(3S)-3-[5-cyclopropyl-3-(2-hydroxyphenyl)pyrrolo[3,2-c]pyridazin-6-yl]pyrrolidin-1-yl]-5-methylpyridin-3-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 110)

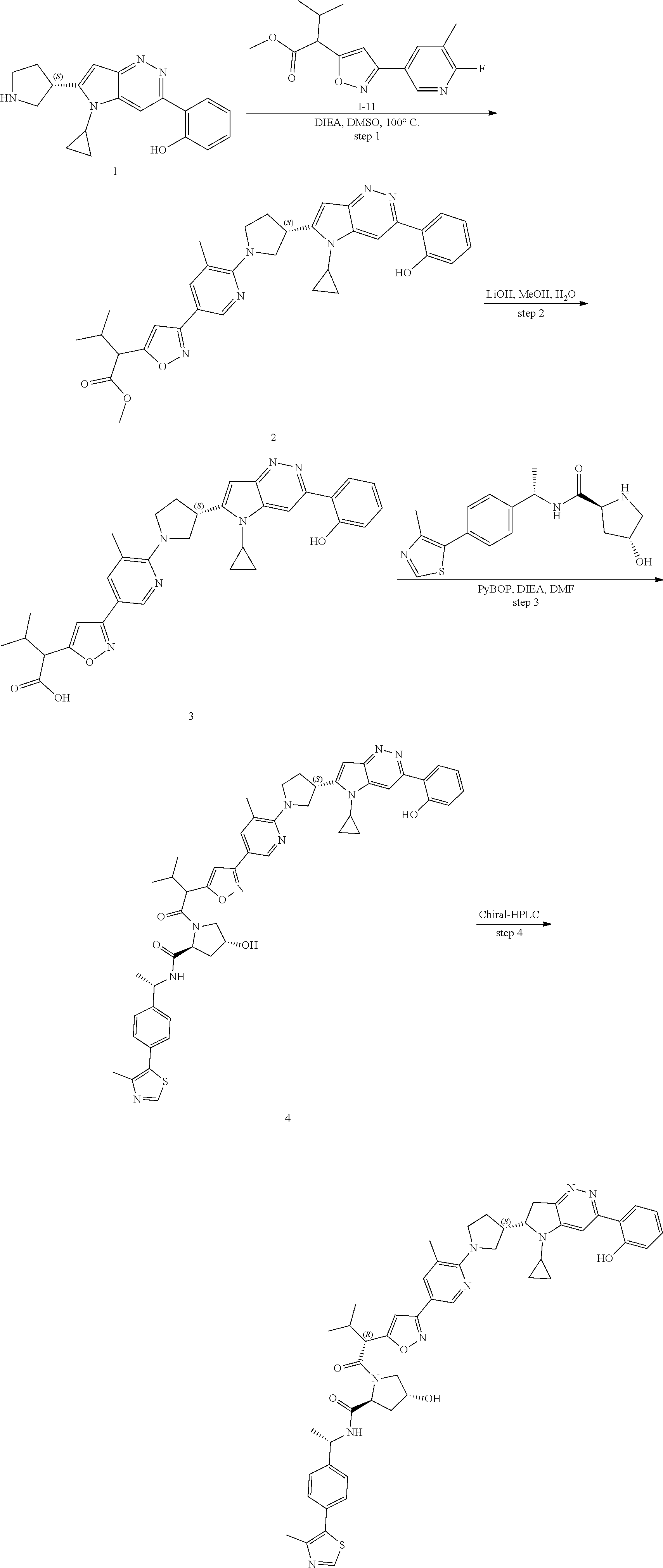

### Step 1: Preparation of methyl 2-(3-{6-[(3S)-3-[5-cyclopropyl-3-(2-hydroxyphenyl)pyrrolo[3,2-c]pyridazin-6-yl]pyrrolidin-1-yl]-5-methylpyridin-3-yl}-1,2-oxazol-5-yl)-3-methylbutanoate (Intermediate 2)

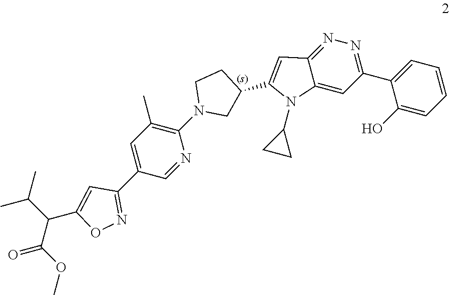 [SMILES: COC(=O)C(c1cc(-c2cnc(N3CC[C@H](c4cc5nnc(-c6ccccc6O)cc5n4C4CC4)C3)c(C)c2)no1)C(C)C]

To a solution of 2-{5-cyclopropyl-6-[(3S)-pyrrolidin-3-yl]pyrrolo[3,2-c]pyridazin-3-yl}phenol (200 mg, 0.624 mmol, 1 equiv) and I-11 (182.47 mg, 0.624 mmol, 1 equiv) in DMSO (5 mL) was added DIEA (242.04 mg, 1.872 mmol, 3 equiv). The resulting solution was stirred at 120 degrees C. for 2 hours. Without additional work-up, the mixture was purified by flash C18 chromatography, elution gradient 0 to 60% acetonitrile in water to give intermediate 2 (110 mg, 19.46%) as a yellow solid. LCMS (ESI) m/z: [M+H]⁺=593.

### Step 2: Preparation of 2-(3-{6-[(3S)-3-[5-cyclopropyl-3-(2-hydroxyphenyl)pyrrolo[3,2-c]pyridazin-6-yl]pyrrolidin-1-yl]-5-methylpyridin-3-yl}-1,2-oxazol-5-yl)-3-methylbutanoic acid (Intermediate 3)

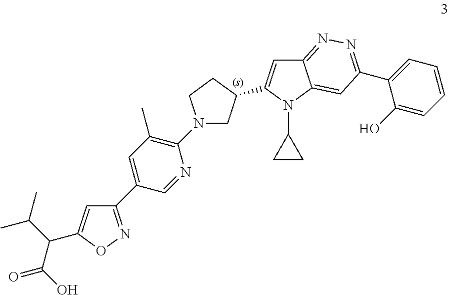 [SMILES: Cc1cc(-c2cc(C(C(=O)O)C(C)C)on2)cnc1N1CC[C@H](c2cc3nnc(-c4ccccc4O)cc3n2C2CC2)C1]

To a solution of intermediate 2 (100 mg, 0.169 mmol, 1 equiv) in methanol (4 mL) were added LiOH (20.20 mg, 0.845 mmol, 5 equiv) and water (1 mL). The resulting solution was stirred at 25 degrees C. for 2 hours. The mixture was acidified to pH 6 with HCl (aq, 1 mol/L). The resulting mixture was extracted with ethyl acetate (3×100 mL). The combined organic layers were washed with brine (3×100 mL), dried over anhydrous Na₂SO₄. After filtration, the filtrate was concentrated under reduced pressure to give intermediate 3 (140 mg, crude) as a yellow solid. LCMS (ESI) m/z: [M+H]⁺=579.

### Step 3: Preparation of (2S,4R)-1-[2-(3-{6-[(3S)-3-[5-cyclopropyl-3-(2-hydroxyphenyl)pyrrolo[3,2-c]pyridazin-6-yl]pyrrolidin-1-yl]-5-methylpyridin-3-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Intermediate 4)

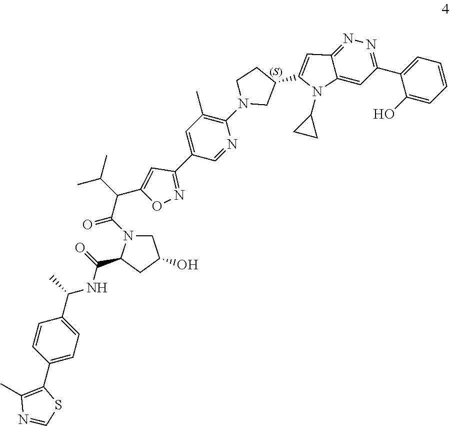

To a solution of intermediate 3 (130 mg, 0.225 mmol, 1 equiv) and (2S,4R)-4-hydroxy-N-[(1S)-1-[4-(4-methyl-1,3-thiazol-5-yl)phenyl]ethyl]pyrrolidine-2-carboxamide (74.46 mg, 0.225 mmol, 1 equiv) in DMF (2 mL) were added DIEA (145.18 mg, 1.125 mmol, 5 equiv) and PyBOP (233.82 mg, 0.450 mmol, 2 equiv). The resulting solution was stirred at 25 degrees C. for 2 hours. Without additional work-up, the crude reaction solution was purified by HPLC (Column: Column: XBridge Shield RP18 OBD Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 2% B to 2% B in 1 min, 2% B to 44% B in 1.5 min, 44% B to 61% B in 8.5 min, 61% B; Wave Length: 254/220 nm; RT1(min): 9.75; Number of Runs: 0 to give intermediate 4 (65 mg, 32.43%) as a yellow solid. LCMS (ESI) m/z: [M+H]⁺=892.

### Step 4: Preparation of (2S,4R)-1-[(2R)-2-(3-{6-[(3S)-3-[5-cyclopropyl-3-(2-hydroxyphenyl)pyrrolo[3,2-c]pyridazin-6-yl]pyrrolidin-1-yl]-5-methylpyridin-3-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 110)

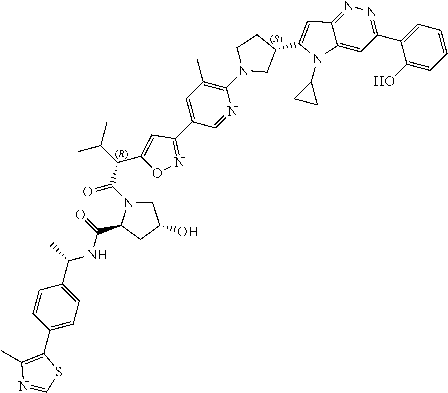

Intermediate 4 (65 mg) was purified by Chiral-HPLC with the following conditions: (Column: XBridge Shield RP18 OBD Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 2% B to 2% B in 1 min, 2% B to 44% B in 1.5 min, 44% B to 61% B in 8.5 min, 61% B; Wave Length: 254/220 nm; RT1(min): 9.75; Number Of Runs: 0). Compound 110-001 (second peak) (21.8 mg, 10.10%) was obtained as a white solid. ¹H NMR (400 MHz, DMSO-d6) δ 14.24 (s, 1H), 8.98 (d, J=2.0 Hz, 1H), 8.48-8.38 (m, 1H), 8.35 (s, 1H), 8.15 (d, J=7.5 Hz, 1H), 7.86-7.80 (m, 1H), 7.78-7.65 (m, 1H), 7.48-7.45 (m, 2H), 7.41-7.28 (m, 2H), 7.25-7.05 (m, 1H), 6.99 (t, J=7.6 Hz, 2H), 6.91 (d, J=5.8 Hz, 1H), 6.85 (s, 1H), 5.10 (d, J=3.6 Hz, 1H), 4.92 (q, J=7.1 Hz, 1H), 4.39 (t, J=7.9 Hz, 1H), 4.30 (s, 1H), 4.20 (dd, J=10.2, 7.1 Hz, 1H), 4.02 (p, J=7.6 Hz, 1H), 3.89-3.79 (m, 4H), 3.76 (dd, J=10.6, 4.4 Hz, 1H), 3.53-3.43 (m, 2H), 2.59-2.52 (m, 1H), 2.48-2.41 (m, 6H), 2.39-2.21 (m, 2H), 2.09-1.99 (m, 1H), 1.95-1.80 (m, 1H), 1.44 (dd, J=39.2, 7.0 Hz, 3H), 1.36-1.30 (m, 2H), 1.26-1.16 (m, 2H), 1.01 (dd, J=6.6, 3.9 Hz, 3H), 0.85 (dd, J=9.9, 6.7 Hz, 3H). LCMS (ESI) m/z: [M+H]⁺=892.20.

The compound in Table 10 was prepared using a procedure similar to the one used above for the preparation of Compound 110 using the appropriate amine.

TABLE 10

| | | LCMS (ESI) | |
|---|---|---|---|
| No. | Name | m/z | ¹H NMR |
| 107 | (2S,4R)-1-((R)-2-(3-(6-((R)-3-(5- | 892.2 | ¹H NMR (300 MHz, DMSO-d6) δ 14.26 (s, |
| | cyclopropyl-3-(2-hydroxyphenyl)- | | 1H), 8.99 (d, J = 1.1 Hz, 1H), 8.50-8.39 |
| | 5H-pyrrolo[3,2-c]pyridazin-6- | | (m, 2H), 8.36 (s, 1H), 8.20-8.11 (m, 1H), |
| | yl)pyrrolidin-1-yl)-5-methylpyridin- | | 7.87-7.80 (m, 1H), 7.50-7.41 (m, 2H), |
| | 3-yl)isoxazol-5-yl)-3- | | 7.39-7.28 (m, 3H), 7.06-6.95 (m, 2H), |
| | methylbutanoyl)-4-hydroxy-N-((S)- | | 6.98-6.91 (m, 1H), 6.89-6.72 (m, 1H), |
| | 1-(4-(4-methylthiazol-5- | | 5.12 (s, 1H), 4.94 (p, J = 7.1 Hz, 1H), 4.40 |
| | yl)phenyl)ethyl)pyrrolidine-2- | | (t, J = 7.9 Hz, 1H), 4.31 (s, 1H), 4.21 (dd, |
| | carboxamide | | J = 10.0, 7.1 Hz, 1H), 4.03 (p, J = 7.5 Hz, |
| | | | 1H), 3.93-3.85 (m, 5H), 3.50 (dd, J = |
| | | | 11.2, 5.5 Hz, 2H), 2.58-2.53 (m, 1H), |
| | | | 2.46 (d, J = 5.0 Hz, 6H), 2.38-2.12 (m, |
| | | | 2H), 1.97 (ddd, J = 12.8, 8.1, 4.8 Hz, 1H), |
| | | | 1.91-1.89 (m, 1H), 1.37 (dd, J = 16.2, 6.9 |
| | | | Hz, 5H), 1.29-1.16 (m, 2H), 1.02 (d, J = |
| | | | 6.5 Hz, 3H), 0.85 (t, J = 7.0 Hz, 3H). |

### Preparation of (2S,4R)-1-[(2R)-2-(3-{2-[(3R)-3-[5-cyclopropyl-3-(2-hydroxyphenyl)pyrrolo[3,2-c]pyridazin-6-yl]pyrrolidin-1-yl]pyrimidin-4-yl}-1,2-oxazol-5-yl)-3-methylbutanoyl]-4-hydroxy-N-[(1 S)-1-[4-(4-methyl-1,3-thiazol-5-yl)phenyl]ethyl]pyrrolidine-2-carboxamide (Compound 116)

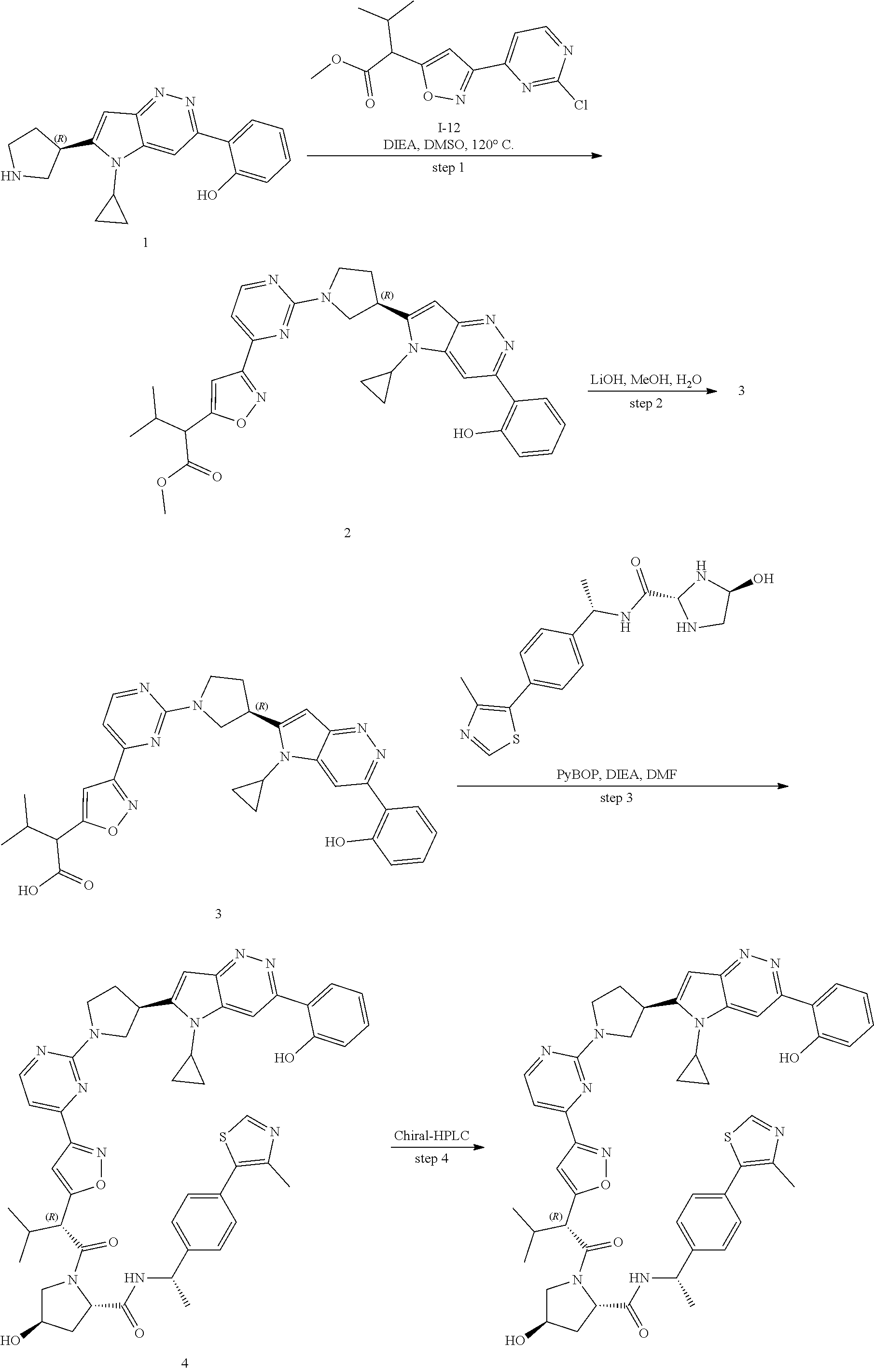

### Step 1: Preparation of methyl 2-(3-{2-[(3R)-3-[5-cyclopropyl-3-(2-hydroxyphenyl)pyrrolo[3,2-c]pyridazin-6-yl]pyrrolidin-1-yl]pyrimidin-4-yl}-1,2-oxazol-5-yl)-3-methylbutanoate (Intermediate 2)

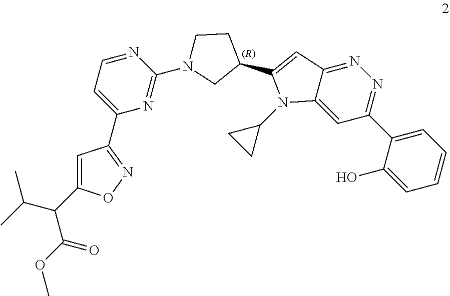

To a stirred solution intermediate 1 (240 mg, 0.749 mmol, 1 equiv) in DMSO (1 mL) were added I-12 (443.03 mg, 1.498 mmol, 2 equiv) and DIEA (484.08 mg, 3.745 mmol, 5 equiv) at room temperature. The resulting mixture was stirred at 120 degrees C. for 2 hours. Desired product could be detected by LCMS. The crude product was purified by flash C18 chromatography, elution gradient 0 to 60% ACN in H₂O to give intermediate 2 (133 mg, 30.63%) as a yellow solid. LCMS (ESI) m/z: [M+H]⁺=580.

### Step 2: Preparation of 2-(3-{2-[(3R)-3-[5-cyclopropyl-3-(2-hydroxyphenyl)pyrrolo[3,2-c]pyridazin-6-yl]pyrrolidin-1-yl]pyrimidin-4-yl}-1,2-oxazol-5-yl)-3-methylbutanoic acid (Intermediate 3)

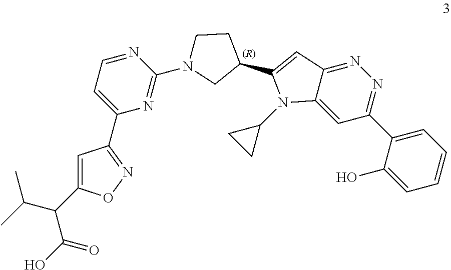

To a solution of methyl intermediate 2 (128 mg, 0.221 mmol, 1 equiv) in MeOH (4 mL) were added H₂O (1 mL) and LiOH (26.44 mg, 1.105 mmol, 5 equiv), the resulting solution was stirred at 25 degrees C. for 2 hours. Desired product could be detected by LCMS. The mixture was acidified to pH 5 with 1 M HCl (aq) and extracted with EtOAc (150 mL×3). The combined organic layers were washed with H₂O (150 mL×3), dried over anhydrous Na₂SO₄. After filtration, the filtrate was concentrated under reduced pressure to give intermediate 3 (126 mg, crude) as a yellow solid. LCMS (ESI) m/z: [M+H]⁺=566.

### Step 3: Preparation of (2S,4R)-1-[2-(3-{2-[(3R)-3-[5-cyclopropyl-3-(2-hydroxyphenyl)pyrrolo[3,2-c]pyridazin-6-yl]pyrrolidin-1-yl]pyrimidin-4-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Intermediate 4)

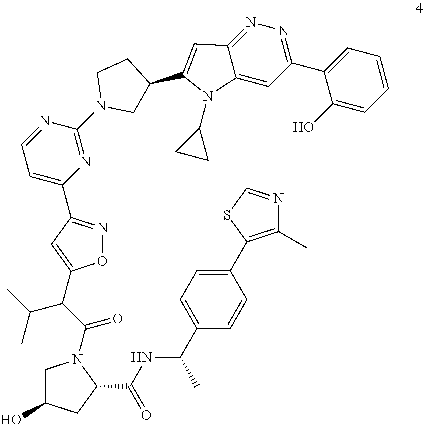 [SMILES: Cc1ncsc1-c1ccc([C@H](C)NC(=O)[C@@H]2C[C@@H](O)CN2C(=O)C(c2cc(-c3ccnc(N4CC[C@@H](c5cc6nnc(-c7ccccc7O)cc6n5C5CC5)C4)n3)no2)C(C)C)cc1]

To a stirred solution of intermediate 3 (126 mg, 0.223 mmol, 1 equiv) and ((2S,4R)-4-hydroxy-N-[(1S)-1-[4-(4-methyl-1,3-thiazol-5-yl)phenyl]ethyl]pyrrolidine-2-carboxamide (147.66 mg, 0.446 mmol, 2 equiv) in DMF (2.8 mL) were added PyBOP (231.85 mg, 0.446 mmol, 2 equiv) and DIEA (143.95 mg, 1.115 mmol, 5 equiv). The resulting mixture was stirred for 2 h at room temperature. Desired product could be detected by LCMS. The mixture was purified by Prep-HPLC with the following conditions: Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 40% B to 65% B in 8 min; Wave Length: 254/220 nm; RT1(min): 9.15 to afford intermediate 4 (66 mg, 33.71%) as a white solid. LCMS (ESI) m/z: [M+H]⁺=879.

### Step 4: Preparation of (2S,4R)-1-[(2R)-2-(3-{2-[(3R)-3-[5-cyclopropyl-3-(2-hydroxyphenyl)pyrrolo[3,2-c]pyridazin-6-yl]pyrrolidin-1-yl]pyrimidin-4-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 116)

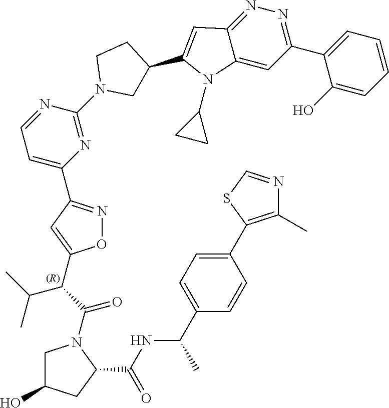

Intermediate 4 (66 mg) was purified by Chiral-Prep-HPLC with the following conditions: Column: CHIRALPAK IE, 2*25 cm, 5 μm; Mobile Phase A: Hex:MtBE=1:1 (0.5% 2M NH₃-MEOH), Mobile Phase B: MeOH--HPLC; Flow rate: 20 mL/min; Gradient: 50% B to 50% B in 25 min; Wave Length: 262/218 nm; RT1(min): 8.46; RT2(min): 15.24; Sample Solvent: EtOH:DCM=1:1-HPLC; Injection Volume: 0.5 mL; Number of Runs: 3. This resulted in compound 116 (16.8 mg, 25.45%) as a white solid. ¹H NMR (300 MHz, DMSO-d6) δ 14.25 (s, 1H), 8.98-8.89 (m, 1H), 8.55 (d, J=4.9 Hz, 1H), 8.40 (d, J=16.0 Hz, 2H), 8.15 (d, J=7.5 Hz, 1H), 7.48-7.33 (m, 5H), 7.28-7.22 (m, 1H) 7.05-6.89 (m, 4H), 5.09-5.01 (m, 1H), 4.91 (s, 1H), 4.38-4.29 (m, 3H), 4.13 (d, J=8.1 Hz, 1H), 3.92-3.81 (m, 2H), 3.74-3.56 (m, 3H), 3.48-3.2 (m, 2H), 2.62-2.56 (m, 1H), 2.45-2.39 (m, 3H), 2.32-2.21 (m, 2H), 2.11-2.01 (m, 1H), 1.78-1.71 (m, 1H), 1.47-1.41 (m, 1H), 1.35 (d, J=6.7 Hz, 4H), 1.21-1.12 (m, 2H), 0.95 (d, J=6.4 Hz, 3H), 0.83 (t, J=7.3 Hz, 3H). LCMS (ESI) m/z: [M+H]⁺=878.37.

### Preparation of (2S,4R)-1-[(2R)-2-(3-{2-[(3R)-3-[5-cyclopropyl-3-(2-hydroxyphenyl) pyrrolo [3,2-c]pyridazin-6-yl]pyrrolidin-1-yl]pyridin-4-yl}-1,2-oxazol-5-yl)-3-methylbutanoyl]-4-hydroxy-N-[(1S)-1-[4-(4-methyl-1,3-thiazol-5-yl) phenyl]ethyl]pyrrolidine-2-carboxamide (Compound 82)

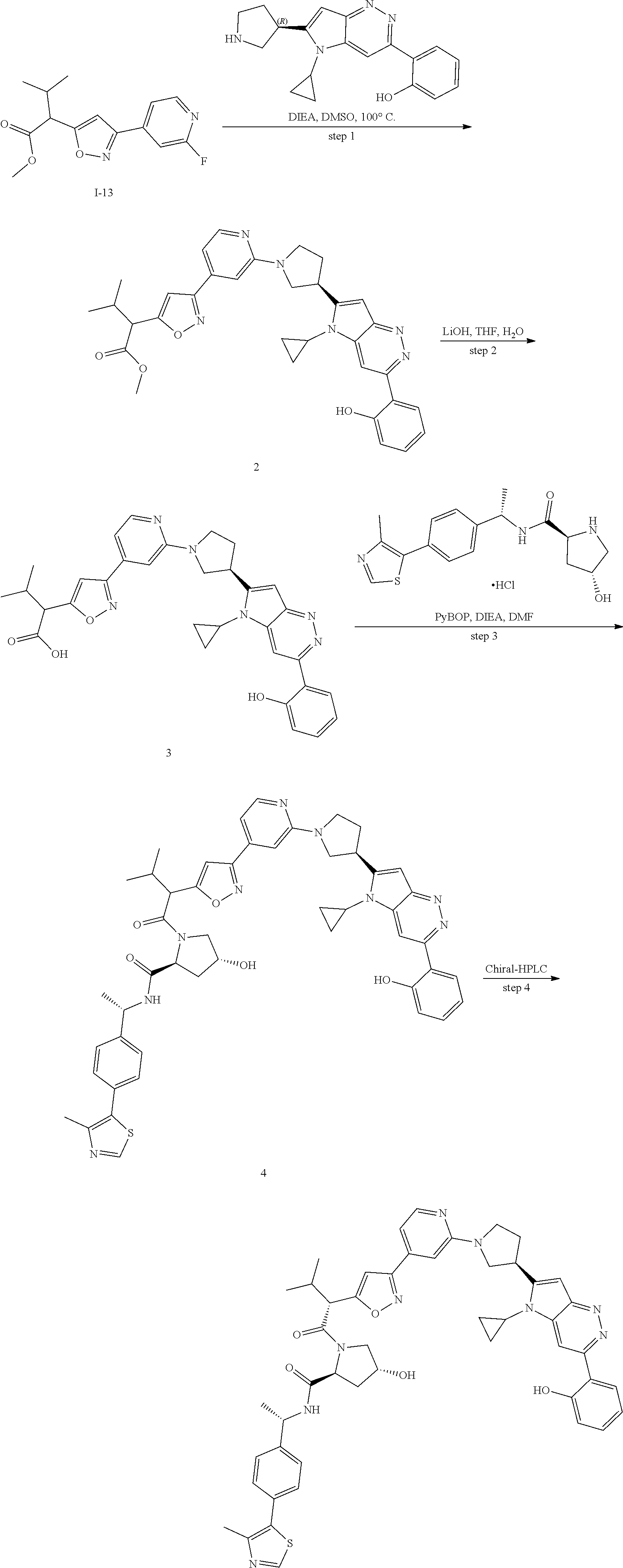

### Step 1: Preparation of methyl 2-(3-{2-[(3R)-3-[5-cyclopropyl-3-(2-hydroxyphenyl) pyrrolo [3,2-c]pyridazin-6-yl]pyrrolidin-1-yl]pyridin-4-yl}-1,2-oxazol-5-yl)-3-methylbutanoate (Intermediate 2)

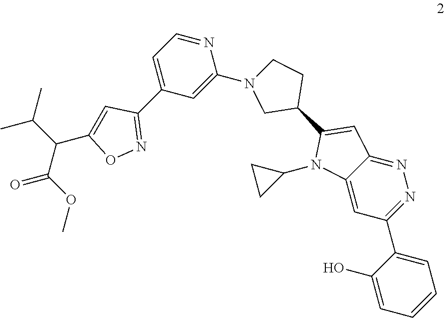 [SMILES: COC(=O)C(c1cc(-c2ccnc(N3CC[C@@H](c4cc5nnc(-c6ccccc6O)cc5n4C4CC4)C3)c2)no1)C(C)C]

Into an 8 mL vial were added 1-13 (87 mg, 0.313 mmol, 1 equiv) and 2-{5-cyclopropyl-6-[(3R)-pyrrolidin-3-yl]pyrrolo [3,2-c]pyridazin-3-yl}phenol (100.17 mg, 0.313 mmol, 1 equiv) and DMSO (3 mL, 42.237 mmol, 135.10 equiv) and DIEA (323.25 mg, 2.504 mmol, 8 equiv) at room temperature. The final reaction mixture for 1 h at 120° C. The reaction was monitored by LCMS. Desired product could be detected by LCMS. This resulted in intermediate 2 (140 mg, 77.39%) as a yellow solid. LCMS (ESI) m/z [M+H]⁺=579.

### Step 2: Preparation of 2-(3-{2-[(3R)-3-[5-cyclopropyl-3-(2-hydroxyphenyl) pyrrolo [3,2-c]pyridazin-6-yl]pyrrolidin-1-yl]pyridin-4-yl}-1,2-oxazol-5-yl)-3-methylbutanoic acid (Intermediate 3)

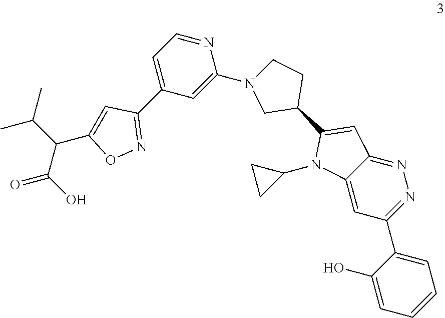 [SMILES: CC(C)C(C(=O)O)c1cc(-c2ccnc(N3CC[C@@H](c4cc5nnc(-c6ccccc6O)cc5n4C4CC4)C3)c2)no1]

Into an 8 mL vial were added intermediate 2 (130 mg, 0.225 mmol, 1 equiv) and LiOH (53.80 mg, 2.250 mmol, 10 equiv) and THF (1.6 mL, 19.748 mmol, 87.91 equiv) and H2O (0.4 mL, 22.204 mmol, 98.84 equiv) at room temperature. The final reaction mixture for 1 h at room temperature. The reaction was monitored by LCMS. Desired product could be detected by LCMS. The mixture was neutralized to pH 6 with conc. HCl. The precipitated solids were collected by filtration and washed with water (3×10 mL). The resulting mixture was concentrated under reduced pressure. This resulted in intermediate 3 (60 mg, 47.30%) as a yellow solid. LCMS (ESI) m/z [M+H]⁺=565.

### Step 3: Preparation of (2S,4R)-1-[(2R)-2-(3-{2-[(3R)-3-[5-cyclopropyl-3-(2-hydroxyphenyl) pyrrolo [3,2-c]pyridazin-6-yl]pyrrolidin-1-yl]pyridin-4-yl}-1,2-oxazol-5-yl)-3-methylbutanoyl]-4-hydroxy-N-[(1S)-1-[4-(4-methyl-1,3-thiazol-5-yl) phenyl]ethyl]pyrrolidine-2-carboxamide (Intermediate 4)

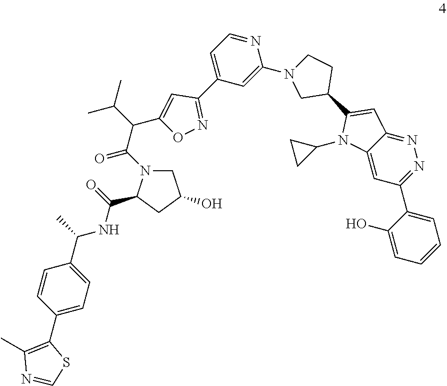 [SMILES: Cc1ncsc1-c1ccc([C@H](C)NC(=O)[C@@H]2C[C@@H](O)CN2C(=O)C(c2cc(-c3ccnc(N4CC[C@@H](c5cc6nnc(-c7ccccc7O)cc6n5C5CC5)C4)c3)no2)C(C)C)cc1]

Into an 8 mL vial were added intermediate 3 (55 mg, 0.097 mmol, 1 equiv) and (2S,4R)-4-hydroxy-N-[(1S)-1-[4-(4-methyl-1,3-thiazol-5-yl) phenyl]ethyl]pyrrolidine-2-carboxamide (32.28 mg, 0.097 mmol, 1 equiv) and PyBOP (76.04 mg, 0.146 mmol, 1.5 equiv) and DMF (3 mL, 38.765 mmol, 397.97 equiv) and DIEA (37.77 mg, 0.291 mmol, 3 equiv) at room temperature. The final reaction mixture for 1 h at room temperature. The reaction was monitored by LCMS. Desired product could be detected by LCMS. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in intermediate 4 (57 mg, 66.64%) as a white solid. LCMS (ESI) m/z [M+H]⁺=878.

### Step 4: Preparation of (2S,4R)-1-[(2R)-2-(3-{2-[(3R)-3-[5-cyclopropyl-3-(2-hydroxyphenyl) pyrrolo [3,2-c]pyridazin-6-yl]pyrrolidin-1-yl]pyridin-4-yl}-1,2-oxazol-5-yl)-3-methylbutanoyl]-4-hydroxy-N-[(1S)-1-[4-(4-methyl-1,3-thiazol-5-yl) phenyl]ethyl]pyrrolidine-2-carboxamide (Compound 82)

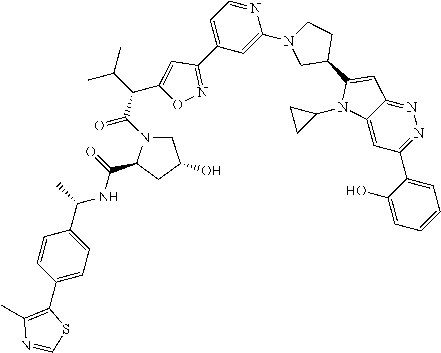 [SMILES: Cc1ncsc1-c1ccc([C@H](C)NC(=O)[C@@H]2C[C@@H](O)CN2C(=O)[C@@H](c2cc(-c3ccnc(N4CC[C@@H](c5cc6nnc(-c7ccccc7O)cc6n5C5CC5)C4)c3)no2)C(C)C)cc1]

Intermediate 4 (57 mg) was purified using Chiral-Prep-HPLC with the following conditions: Column: CHIRAL ART Cellulose-SB, 2*25 cm, 5 μm; Mobile Phase A: MtBE (10 mM NH3-MeOH), Mobile Phase B: MeOH--HPLC; Flow rate: 20 mL/min; Gradient: 30% B to 30% B in 16 min; Wave Length: 264/244 nm; RT1(min): 6.25; RT2(min): 10.5; Sample Solvent: MeOH:DCM=2:1; Injection Volume: 1 mL; This resulted in Compound 82 (17.1 mg, 19.73%) as a white solid to give. 1H NMR (400 MHz, DMSO-d6) δ 14.26 (s, 1H), 8.98 (d, J=2.3 Hz, 1H), 8.43 (d, J=7.7 Hz, 1H), 8.37 (s, 1H), 8.22 (d, J=5.2 Hz, 1H), 8.16 (dd, J=8.0, 1.7 Hz, 1H), 7.47-7.40 (m, 2H), 7.40-7.29 (m, 3H), 7.12-6.94 (m, 5H), 6.91 (d, J=4.2 Hz, 1H), 5.11 (d, J=3.6 Hz, 1H), 5.06-4.86 (m, 1H), 4.39 (t, J=7.9 Hz, 1H), 4.30 (s, 1H), 4.21-4.05 (m, 2H), 3.89 (d, J=9.7 Hz, 1H), 3.85-3.71 (m, 2H), 3.73-3.58 (m, 2H), 3.55-3.44 (m, 2H), 2.70-2.57 (m, 1H), 2.45 (d, J=8.4 Hz, 3H), 2.40-2.29 (m, 2H), 2.04 (t, J=10.5 Hz, 1H), 1.86-1.70 (m, 1H), 1.57-1.28 (m, 5H), 1.27-1.15 (m, 2H), 1.02 (dd, J=6.6, 2.7 Hz, 3H), 0.85 (t, J=7.6 Hz, 3H). LCMS (ESI) m/z [M+H]+=878.07.

The compound in Table 11 was prepared using a procedure similar to the one used above for the preparation of Compound 82 using the appropriate amine.

TABLE 11

| | | LCMS (ESI) | |
|---|---|---|---|
| No. | Name | m/z | ¹H NMR |
| 83 | (2S,4R)-1-((R)-2-(3-(2-((S)-3-(5- | 878.45 | ¹H NMR (300 MHz, DMSO-d6) δ 14.24 (s, 1H), |
| | cyclopropyl-3-(2-hydroxyphenyl)- | | 8.99 (s, 1H), 8.49-8.33 (m, 2H), 8.19 (dd, J = |
| | 5H-pyrrolo[3,2-c]pyridazin-6- | | 19.0, 6.5 Hz, 2H), 7.50-7.26 (m, 5H), 7.10- |
| | yl)pyrrolidin-1-yl)pyridin-4- | | 6.88 (m, 5H), 6.83 (s, 1H), 5.11-4.91 (m, 2H), |
| | yl)isoxazol-5-yl)-3-methylbutanoyl)- | | 4.39 (t, J = 7.9 Hz, 1H), 4.30 (s, 1H), 4.16 (d, |
| | 4-hydroxy-N-((S)-1-(4-(4- | | J = 9.1 Hz, 2H), 3.93-3.73 (m, 3H), 3.71-3.49 |
| | methylthiazol-5- | | (m, 4H), 2.63 (s, 1H), 2.44 (d, J = 6.7 Hz, 3H), |
| | yl)phenyl)ethyl)pyrrolidine-2- | | 2.36 (d, J = 10.5 Hz, 2H), 2.04 (s, 1H), 1.79 (s, |
| | carboxamide | | 1H), 1.49 (d, J = 6.9 Hz, 1H), 1.37 (t, J = 7.8 |
| | | | Hz, 4H), 1.22 (d, J = 7.2 Hz, 2H), 1.02 (d, J = |
| | | | 6.5 Hz, 3H), 0.84 (t, J = 6.2 Hz, 3H). |

### Preparation of (2S,4R)-1-((R)-2-(3-(4-((R)-3-(5-cyclopropyl-3-(2-hydroxyphenyl)-5H-pyrrolo[3,2-c]pyridazin-6-yl)pyrrolidin-1-yl)pyridin-2-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 91)

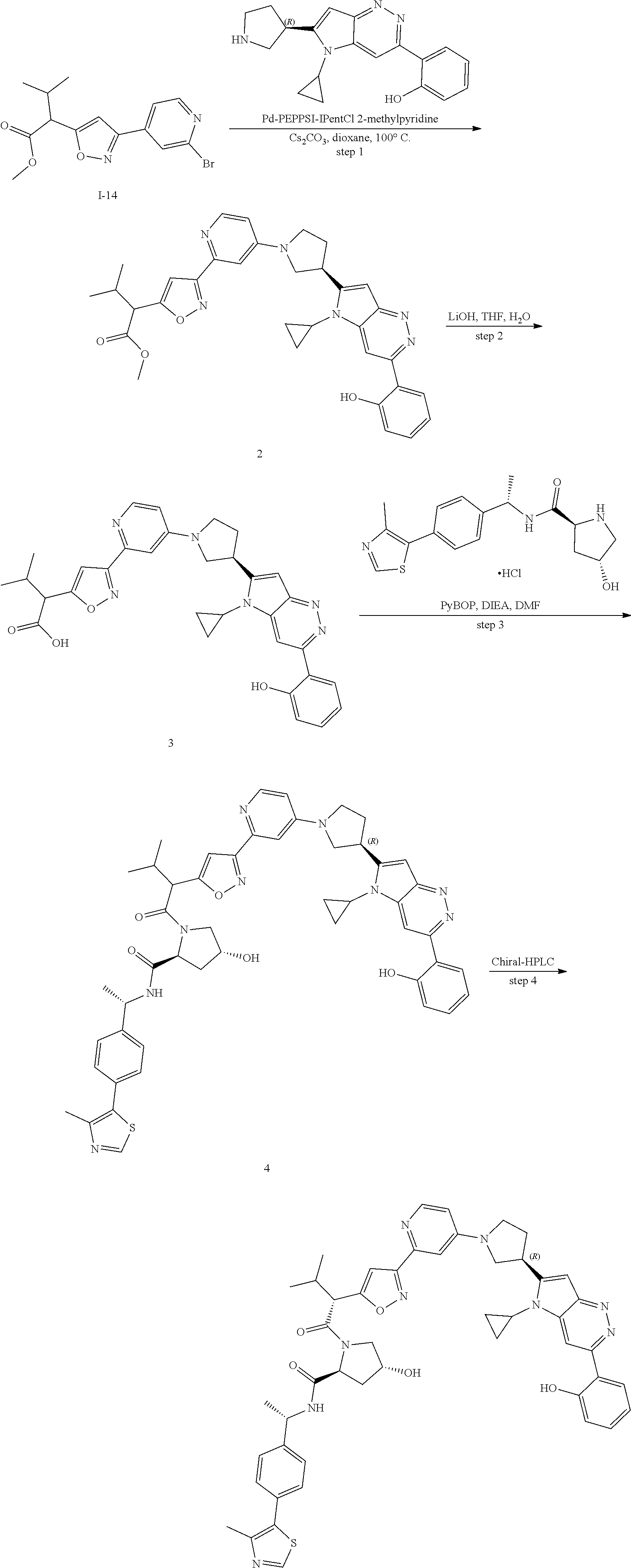

### Step 1: Preparation of methyl 2-(3-{4-[(3R)-3-[5-cyclopropyl-3-(2-hydroxyphenyl)pyrrolo[3,2-c]pyridazin-6-yl]pyrrolidin-1-yl]pyridin-2-yl}-1,2-oxazol-5-yl)-3-methylbutanoate (Intermediate 2)

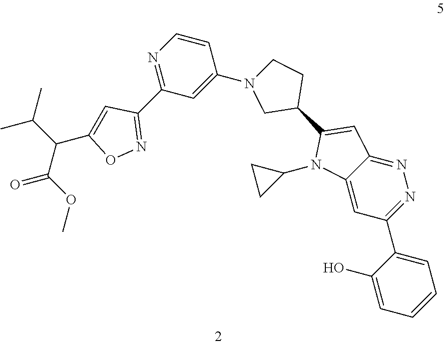 [SMILES: COC(=O)C(c1cc(-c2cc(N3CC[C@@H](c4cc5nnc(-c6ccccc6O)cc5n4C4CC4)C3)ccn2)no1)C(C)C]

To a stirred solution of 1-14 (200.0 mg, 0.590 mmol, 1 equiv) and 2-{5-cyclopropyl-6-[(3R)-pyrrolidin-3-yl]pyrrolo[3,2-c]pyridazin-3-yl}phenol (188.9 mg, 0.590 mmol, 1 equiv) in 1,4-dioxane (5 mL) were added Cs₂CO₃ (576.3 mg, 1.770 mmol, 3 equiv) and Pd-PEPPSI-IPentCI 2-methylpyridine (o-picoline) (24.0 mg, 0.029 mmol, 0.05 equiv). The resulting mixture was stirred for 2 h at 100° C. under nitrogen atmosphere. The mixture was concentrated under vacuum, the residue was purified by reversed-phase flash chromatography with the following conditions: column, C₁₈ silica gel; mobile phase, MeCN in Water (0.1% FA), 30% to 70% gradient in 30 min; detector, UV 254 nm. This resulted in intermediate 2 (210.0 mg, 61.5%) as a yellow solid. LCMS (ESI) m/z: [M+H]⁺=579.

### Step 2: Preparation of 2-(3-{4-[(3R)-3-[5-cyclopropyl-3-(2-hydroxyphenyl)pyrrolo[3,2-c]pyridazin-6-yl]pyrrolidin-1-yl]pyridin-2-yl}-1,2-oxazol-5-yl)-3-methylbutanoic acid (Intermediate 3)

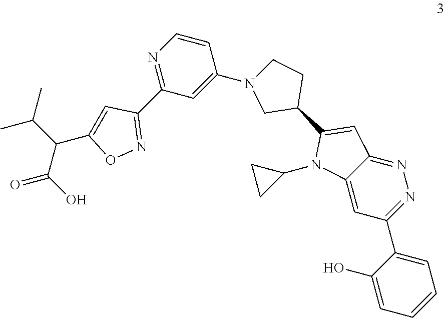 [SMILES: CC(C)C(C(=O)O)c1cc(-c2cc(N3CC[C@@H](c4cc5nnc(-c6ccccc6O)cc5n4C4CC4)C3)ccn2)no1]

To a stirred solution of intermediate 2 (210.0 mg, 0.363 mmol, 1 equiv) in THF (5 mL) and H₂O (5 mL) was added LiOH (86.9 mg, 3.630 mmol, 10 equiv). The resulting mixture was stirred for 1 h at room temperature. The mixture was neutralized to pH 7 with 1 N HCl, the precipitated solids were collected by filtration and washed with water (3×1 mL). This resulted in intermediate 3 (180.0 mg, crude) as a yellow solid. The crude product was used in the next step directly without further purification. LCMS (ESI) m/z [M+H]⁺=565.

### Step 3: Preparation of (2S,4R)-1-((R)-2-(3-(4-((R)-3-(5-cyclopropyl-3-(2-hydroxyphenyl)-5H-pyrrolo[3,2-c]pyridazin-6-yl)pyrrolidin-1-yl)pyridin-2-yl)isoxazol-5-yl)-3-methylbutanoyl)-4-hydroxy-N—((S)-1-(4-(4-methylthiazol-5-yl)phenyl)ethyl)pyrrolidine-2-carboxamide (Intermediate 4)

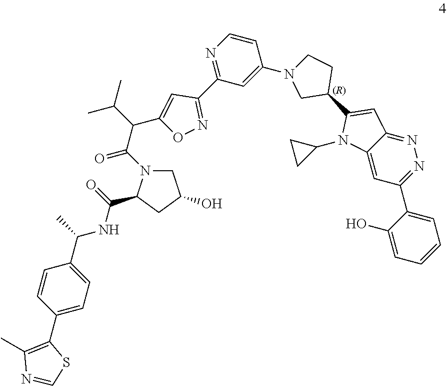

To a stirred solution of intermediate 3 (100.0 mg, 0.177 mmol, 1 equiv) and (2S,4R)-4-hydroxy-N-[(1S)-1-[4-(4-methyl-1,3-thiazol-5-yl)phenyl]ethyl]pyrrolidine-2-carboxamide (70.4 mg, 0.212 mmol, 1.2 equiv) in DMF (5 ml) were added PyBOP (184.3 mg, 0.354 mmol, 2 equiv) and DIEA (137.3 mg, 1.062 mmol, 6 equiv), the resulting solution was stirred at room temperature for 2 h. The mixture was purified by reversed-phase flash chromatography with the following conditions: column, C₁₈ silica gel; mobile phase, MeCN in Water (0.1% FA), 30% to 100% gradient in 35 min; detector, UV 254 nm. This resulted in intermediate 4 (78.0 mg, 50.1%) as a yellow solid. LCMS (ESI) m/z: [M+H]⁺=878.

### Step 4: Preparation of (2S,4R)-1-((R)-2-(3-(4-((R)-3-(5-cyclopropyl-3-(2-hydroxyphenyl)-5H-pyrrolo[3,2-c]pyridazin-6-yl)pyrrolidin-1-yl)pyridin-2-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 91)

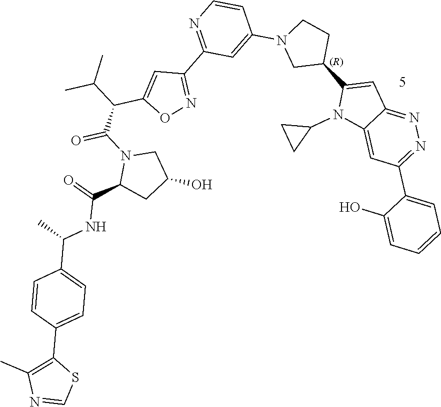

Intermediate 4 (78.0 mg) was separated by Chiral-Prep-HPLC with the following conditions: Column: CHIRAL ART Cellulose-SB, 2*25 cm, 5 μm; Mobile Phase A: MtBE (10 mM NH3-MeOH), Mobile Phase B: MeOH--HPLC; Flow rate: 20 mL/min; Gradient: 30% B to 30% B in 16 min; Wave Length: 264/244 nm; RT1(min): 6.25; RT2(min): 10.5; Sample Solvent: MeOH:DCM=2:1; Injection Volume: 1 mL; Number Of Runs: 3 to afford 91-001 (second peak) (17.6 mg, 34.9%) as a white solid. ¹H NMR (400 MHz, DMSO-d6) δ 14.23 (s, 1H), 9.01-8.96 (m, 1H), 8.44 (d, J=7.7 Hz, 1H), 8.37 (s, 1H), 8.26 (d, J=5.8 Hz, 1H), 8.19-8.12 (m, 1H), 7.48-7.29 (m, 5H), 7.19-7.12 (m, 1H), 7.04-6.90 (m, 3H), 6.84 (s, 1H), 6.72-6.66 (m, 1H), 5.11 (d, J=3.7 Hz, 1H), 4.99-4.87 (m, 1H), 4.43-4.34 (m, 1H), 4.32-4.27 (m, 1H), 4.19-4.08 (m, 1H), 4.08-3.99 (m, 1H), 3.92-3.84 (m, 1H), 3.79-3.47 (m, 6H), 2.70-2.60 (m, 1H), 2.38-2.25 (m, 5H), 2.10-1.98 (m, 1H), 1.84-1.73 (m, 1H), 1.49 (d, J=6.9 Hz, 1H), 1.42-1.31 (m, 4H), 1.24-1.19 (m, 2H), 1.05-0.91 (m, 3H), 0.89-0.79 (m, 3H). LCMS (ESI) m/z: [M+H]⁺=878.35.

### Preparation of (2S,4R)-1-[(2R)-2-(3-{6-[5-cyclobutyl-3-(2-hydroxyphenyl)pyrrolo[3,2-c]pyridazin-6-yl]-2-azaspiro[3.3]heptan-2-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carb oxamide (Compound 105)

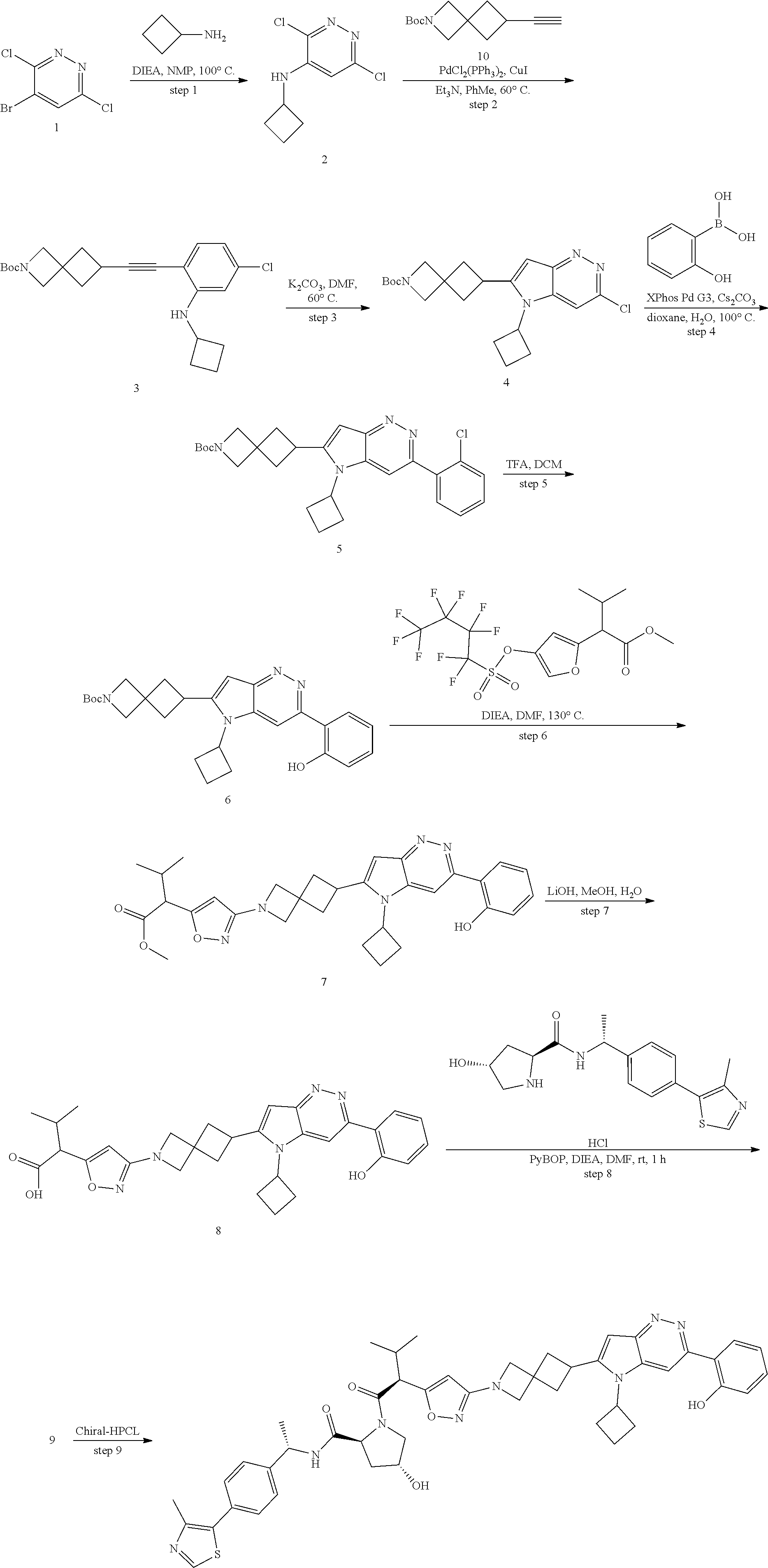

### Step 1: 3,6-dichloro-N-cyclobutylpyridazin-4-amine (intermediate 2)

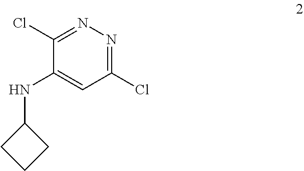 [SMILES: Clc1cc(NC2CCC2)c(Cl)nn1]

Into a 250 mL round-bottom flask were added 4-bromo-3,6-dichloropyridazine (10 g, 43.885 mmol, 1 equiv) and cyclobutylamine (3.43 g, 48.273 mmol, 1.1 equiv) and NMP (88 mL, 912.552 mmol, 20.79 equiv) and DIEA (11.34 g, 87.770 mmol, 2 equiv) at room temperature. The final reaction mixture for 1 h at 100° C. The reaction was monitored by LCMS. The resulting mixture was diluted with water (100 mL). The resulting mixture was extracted with CH2Cl2 (50×mL). The combined organic layers were washed with water (3×50 mL), dried over anhydrous Na2SO4. After filtration, the filtrate was concentrated under reduced pressure. The residue was purified by silica gel column chromatography, eluted with PE/EA (1:1) to afford intermediate 2 (8.8893 g, 92.88%) as a reddish solid. LCMS (ESI) m/z [M+H]⁺=218.

### Step 2: Preparation of tert-butyl 6-{2-[6-chloro-4-(cyclobutylamino) pyridazin-3-yl]ethynyl}-2-azaspiro[3.3]heptane-2-carboxylate (Intermediate 3)

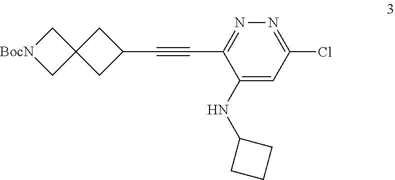 [SMILES: CC(C)(C)OC(=O)N1CC2(CC(C#Cc3nnc(Cl)cc3NC3CCC3)C2)C1]

A mixture of intermediate 3 (1.7935 g, 8.224 mmol, 1 equiv) and tert-butyl 6-ethynyl-2-azaspiro[3.3]heptane-2-carboxylate (2.00 g, 9.046 mmol, 1.1 equiv) and CuI (0.31 g, 1.645 mmol, 0.2 equiv) and dichloropalladium; bis(triphenylphosphane) (1.15 g, 1.645 mmol, 0.2 equiv) and Et₃N (2.50 g, 24.672 mmol, 3 equiv) in toluene was stirred for 1 h at 60° C. under nitrogen atmosphere. The reaction was monitored by LCMS. The resulting mixture was diluted with MeOH (50 mL). The residue was purified by silica gel column chromatography, eluted with PE/EA (1:1) to afford intermediate 3 (1.2 g, 36.21%) as a yellow solid. LCMS (ESI) m/z [M+H]⁺=403

### Step 3: Preparation of tert-butyl 6-{3-chloro-5-cyclobutylpyrrolo [3,2-c]pyridazin-6-yl}-2-azaspiro [3.3]heptane-2-carboxylate (intermediate 4)

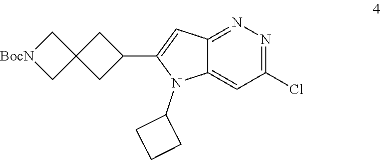 [SMILES: CC(C)(C)OC(=O)N1CC2(CC(c3cc4nnc(Cl)cc4n3C3CCC3)C2)C1]

Into an 8 mL vial were added intermediate 3 (40 mg, 0.099 mmol, 1 equiv) and K₂CO₃ (41.16 mg, 0.297 mmol, 3 equiv) and DMF (1 mL, 12.922 mmol, 130.16 equiv) at room temperature. The final reaction mixture for 1 h at 100° C. The reaction was monitored by LCMS. Desired product could be detected by LCMS. The residue was purified by reversed-phase flash chromatography with the following conditions: column, C18 silica gel; mobile phase, MeCN in Water (10 mmol/L NH4HCO3), 10% to 50% gradient in 10 min; detector, UV 254 nm. This resulted in intermediate 4 (384 mg, 34.91%) as a yellow solid. LCMS (ESI) m/z [M+H]⁺=403

### Step 4: preparation of tert-butyl 6-[5-cyclobutyl-3-(2-hydroxyphenyl) pyrrolo[3,2-c]pyridazin-6-yl]-2-azaspiro[3.3]heptane-2-carboxylate (intermediate 5)

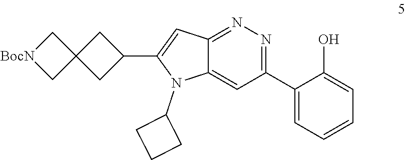 [SMILES: CC(C)(C)OC(=O)N1CC2(CC(c3cc4nnc(-c5ccccc5O)cc4n3C3CCC3)C2)C1]

A solution of 4 (354 mg, 0.879 mmol, 1 equiv) and 2-hydroxyphenylboronic acid (363.55 mg, 2.637 mmol, 3 equiv) and XPhos Pd G3 (148.74 mg, 0.176 mmol, 0.2 equiv) and Cs2CO3 (858.78 mg, 2.637 mmol, 3 equiv) in dioxane (2.5 mL, 29.510 mmol, 33.59 equiv) and H2O (0.5 mL, 27.755 mmol, 31.59 equiv) was stirred for 1 h at room temperature under nitrogen atmosphere. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in intermediate 5 (337 mg, 83.28%) as a yellow solid. LCMS (ESI) m/z [M+H]⁺=461

### Step 5: preparation of 2-(6-{2-azaspiro[3.3]heptan-6-yl}-5-cyclobutylpyrrolo[3,2-c]pyridazin-3-yl)phenol (Intermediate 6)

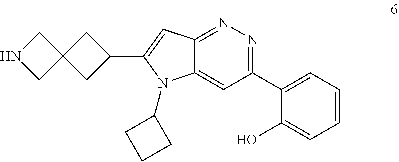 [SMILES: Oc1ccccc1-c1cc2c(cc(C3CC4(CNC4)C3)n2C2CCC2)nn1]

Into an 8 mL vial were added intermediate 5 (50 mg, 0.109 mmol, 1 equiv) and TFA (0.5 mL, 6.732 mmol, 62.01 equiv) and DCM (1 mL, 15.731 mmol, 144.90 equiv) at room temperature. The reaction was monitored by LCMS. The resulting mixture was concentrated under reduced pressure. The resulting oil was dried under vacuum. This resulted in intermediate 6 (417 mg, 92.83%) as a brown yellow solid. LCMS (ESI) m/z [M+H]⁺=361

### Step 6: preparation of methyl 2-(3-{6-[5-cyclobutyl-3-(2-hydroxyphenyl) pyrrolo[3,2-c]pyridazin-6-yl]-2-azaspiro [3.3]heptan-2-yl}-1,2-oxazol-5-yl)-3-methylbutanoate (Intermediate 7)

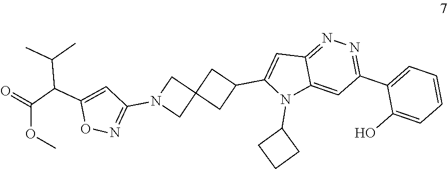 [SMILES: COC(=O)C(c1cc(N2CC3(CC(c4cc5nnc(-c6ccccc6O)cc5n4C4CCC4)C3)C2)no1)C(C)C]

Into an 8 mL vial were added intermediate 6 (405 mg, 1.124 mmol, 1 equiv) and methyl 3-methyl-2-{3-[(1,1,2,2,3,3,4,4,4-nonafluorobutanesulfonyl) oxy]-1,2-oxazol-5-yl}butanoate (540.76 mg, 1.124 mmol, 1 equiv) and DMF (7 mL, 90.451 mmol, 80.50 equiv) and DIEA (726.08 mg, 5.620 mmol, 5 equiv) at room temperature. The final reaction mixture for 2 h at 120° C. The reaction was monitored by LCMS. The residue/crude product was purified by reverse phase flash with the following conditions (MeCN/Water) to afford intermediate 7 (85 mg, 13.97%) as a brown yellow solid. LCMS (ESI) m/z [M+H]⁺=542.

### Step 7: preparation of 2-(3-{6-[5-cyclobutyl-3-(2-hydroxyphenyl) pyrrolo [3,2-c]pyridazin-6-yl]-2-azaspiro [3.3]heptan-2-yl}-1,2-oxazol-5-yl)-3-methylbutanoic acid (Intermediate 8)

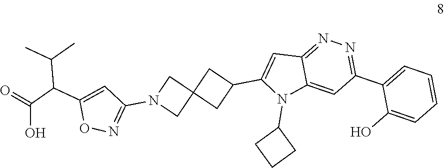 [SMILES: CC(C)C(C(=O)O)c1cc(N2CC3(CC(c4cc5nnc(-c6ccccc6O)cc5n4C4CCC4)C3)C2)no1]

Into an 8 mL vial were added intermediate 7 (8 mg, 0.015 mmol, 1 equiv) and LiOH (3.54 mg, 0.150 mmol, 10 equiv) and MeOH (0.9 mL, 22.229 mmol, 1505.04 equiv) and H2O (0.3 mL, 16.653 mmol, 1127.50 equiv) at room temperature. The final reaction mixture for 1 h at 40° C. The reaction was monitored by LCMS. The mixture/residue was acidified to pH 6 with conc. HCl. The precipitated solids were collected by filtration and washed with water (1×3 mL). The resulting mixture was concentrated under reduced pressure. This resulted in intermediate 8 (120 mg, 159.99%) as a yellow solid. LCMS (ESI) m/z [M+H]⁺=528.

### Step 8: preparation of (2S,4R)-1-[(2R)-2-(3-{6-[5-cyclobutyl-3-(2-hydroxyphenyl) pyrrolo [3,2-c]pyridazin-6-yl]-2-azaspiro [3.3]heptan-2-yl}-1,2-oxazol-5-yl)-3-methylbutanoyl]-4-hydroxy-N-[(1S)-1-[4-(4-methyl-1,3-thiazol-5-yl) phenyl]ethyl]pyrrolidine-2-carboxamide (Intermediate 9)

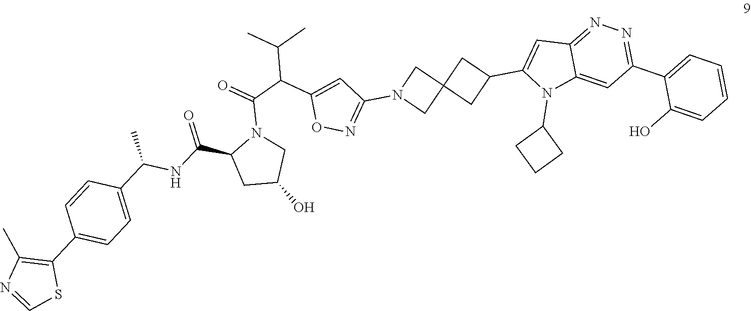 [SMILES: Cc1ncsc1-c1ccc([C@H](C)NC(=O)[C@@H]2C[C@@H](O)CN2C(=O)C(c2cc(N3CC4(CC(c5cc6nnc(-c7ccccc7O)cc6n5C5CCC5)C4)C3)no2)C(C)C)cc1]

To a stirred solution/mixture of intermediate 8 (26 mg, 0.049 mmol, 1 equiv) and (2S,4R)-4-hydroxy-N-[(1S)-1-[4-(4-methyl-1,3-thiazol-5-yl) phenyl]ethyl]pyrrolidine-2-carboxamide (16.33 mg, 0.049 mmol, 1 equiv) and PyBOP (38.47 mg, 0.074 mmol, 1.5 equiv) in DMF was added DIEA (15.92 mg, 0.122 mmol, 2.5 equiv) dropwise at room temperature under nitrogen atmosphere. The final reaction mixture for 1 h at room temperature. The reaction was monitored by LCMS. The residue product was purified by reverse phase flash with the following conditions (MeCN/Water (0.1% FA)) to afford intermediate 9 (8.7 mg, 20.09%) as a whit e solid. LCMS (ESI) m/z [M+H]⁺=841.

### Step 9: preparation of (2S,4R)-1-[(2R)-2-(3-{6-[5-cyclobutyl-3-(2-hydroxyphenyl) pyrrolo [3,2-c]pyridazin-6-yl]-2-azaspiro [3.3]heptan-2-yl}-1,2-oxazol-5-yl)-3-methylbutanoyl]-4-hydroxy-N-[(1S)-1-[4-(4-methyl-1,3-thiazol-5-yl) phenyl]ethyl]pyrrolidine-2-carboxamide (Compound 105-001)

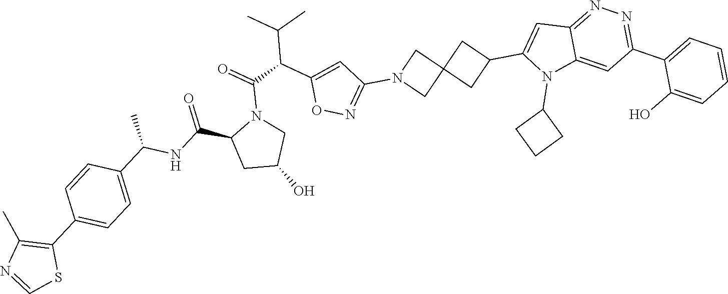 [SMILES: Cc1ncsc1-c1ccc([C@H](C)NC(=O)[C@@H]2C[C@@H](O)CN2C(=O)[C@@H](c2cc(N3CC4(CC(c5cc6nnc(-c7ccccc7O)cc6n5C5CCC5)C4)C3)no2)C(C)C)cc1]

The intermediate 9 (26 mg) was purified by Chiral-Prep-HPLC with the following conditions (NB-rep Chiral HPLC-02): Column, CHIRALPAK IG, 2*25 cm, 5 um; mobile phase, MtBE (10 mM NH3-MeOH) and MeOH- (hold 30% MeOH- in 16 min) The resulting mixture was concentrated under reduced pressure to afford compound 105 (8.7 mg, 20.09%) as a white solid 1H NMR (400 MHz, DMSO-d6) δ 14.16 (s, 1H), 8.99 (d, J=2.8 Hz, 1H), 8.46-8.41 (m, 2H), 8.25-8.05 (m, 1H), 7.51-7.41 (m, 2H), 7.37 (d, J=8.3 Hz, 2H), 7.33-7.31 (m, 1H), 7.12-6.92 (m, 2H), 6.87 (s, 1H), 5.81 (d, J=43.6 Hz, 1H), 5.10 (d, J=3.5 Hz, 1H), 4.97-4.86 (m, 2H), 4.37 (t, J=7.8 Hz, 1H), 4.29 (s, 1H), 4.03 (s, 2H), 3.84 (s, 2H), 3.79-3.67 (m, 2H), 3.59 (d, J=9.7 Hz, 1H), 3.53-3.39 (m, 2H), 2.88 (td, J=9.8, 2.6 Hz, 2H), 2.84-2.75 (m, 2H), 2.52 (s, 1H), 2.49-2.44 (m, 5H), 2.36-2.12 (m, 1H), 2.04 (q, J=7.2 Hz, 2H), 1.85 (dt, J=22.3, 10.7 Hz, 2H), 1.60-1.16 (m, 3H), 0.95 (d, J=6.4 Hz, 3H), 0.80 (t, J=7.4 Hz, 3H). LCMS (ESI) m/z [M+H]⁺=841.04.

Compounds in Table 12 were prepared using procedures similar to the one used above for the preparation of Compound 105 using the appropriate amines and alkynes.

TABLE 12

| | | LCMS (ESI) | |
|---|---|---|---|
| No. | Name | m/z | ¹H NMR |
| 48 | (2S,4R)-1-((R)-2-(3-(6-(5- | 827.30 | ¹H NMR (300 MHz, DMSO-d6) δ 14.16 (s, 1H), |
| | cyclobutyl-3-(2-hydroxyphenyl)-5H- | | 8.99 (s, 1H), 8.50 (d, J = 6.0 Hz, 1H), 8.42 (s, |
| | pyrrolo[3,2-c]pyridazin-6-yl)-2- | | 1H), 8.17 (d, J = 7.5 Hz, 1H), 7.48-7.26 (m, |
| | azaspiro[3.3]heptan-2-yl)isoxazol- | | 6H), 7.04-6.94 (m, 2H), 6.86 (d, J = 6.0 Hz, |
| | 5-yl)-3-methylbutanoyl)-4-hydroxy- | | 1H), 5.87 (s, 1H), 5.12 (d, J = 3.6 Hz, 1H), 4.97- |
| | N-(4-(4-methylthiazol-5- | | 4.85 (m, 1H), 4.35 (d, J = 6.0 Hz, 3H), 4.03 (s, |
| | yl)benzyl)pyrrolidine-2- | | 2H), 3.81 (d, J = 16.7 Hz, 4H), 3.61 (d, J = 9.7 |
| | carboxamide | | Hz, 1H), 3.44 (s, 1H), 2.94-2.73 (m, 5H), 2.44 |
| | | | (d, J = 5.3 Hz, 3H), 2.31-2.15 (m, 3H), 2.04 (d, |
| | | | J = 9.2 Hz, 5H), 0.94 (d, J = 6.5 Hz, 3H), 0.79 (d, |
| | | | J = 6.7 Hz, 3H). |
| 61 | (2S,4R)-1-((R)-2-(3-(6-(5- | 654.4 | ¹H NMR (300 MHz, DMSO-d6) δ 14.17 (s, 1H), |
| | cyclobutyl-3-(2-hydroxyphenyl)-5H- | | 8.42 (s, 1H), 8.22-8.10 (m, 1H), 7.85 (d, J = 4.7 |
| | pyrrolo[3,2-c]pyridazin-6-yl)-2- | | Hz, 1H), 7.39-7.25 (m, 1H), 7.05-6.92 (m, 2H), |
| | azaspiro[3.3]heptan-2-yl)isoxazol- | | 6.87 (s, 1H), 5.85 (s, 1H), 5.10 (d, J = 3.6 Hz, |
| | 5-yl)-3-methylbutanoyl)-4-hydroxy- | | 1H), 5.03-4.83 (m, 1H), 4.32 (s, 1H), 4.22 (t, |
| | N-methylpyrrolidine-2-carboxamide | | J = 7.9 Hz, 1H), 4.03 (s, 2H), 3.86-3.67 (m, 4H), |
| | | | 3.58 (d, J = 9.8 Hz, 2H), 2.98-2.81 (m, 2H), 2.81- |
| | | | 2.73 (m, 2H), 2.57 (d, J = 4.5 Hz, 3H), 2.44 (s, |
| | | | 3H), 2.27-1.76 (m, 6H), 1.01-0.85 (m, 3H), |
| | | | 0.83-0.74 (m, 3H). |
| 65 | (2S,4R)-1-((R)-2-(3-(6-(5- | 853.3 | ¹H NMR (400 MHz, DMSO-d6) δ 14.06 (s, 1H), |
| | (bicyclo[1.1.1]pentan-1-yl)-3-(2- | | 8.99 (d, J = 1.7 Hz, 1H), 8.42 (d, J = 7.7 Hz, 1H), |
| | hydroxyphenyl)-5H-pyrrolo[3,2- | | 8.31 (s, 1H), 8.12 (dd, J = 8.3, 1.6 Hz, 1H), 7.52- |
| | c]pyridazin-6-yl)-2- | | 7.41 (m, 2H), 7.40-7.27 (m, 3H), 7.04-6.91 |
| | azaspiro[3.3]heptan-2-yl)isoxazol- | | (m, 3H), 5.81 (d, J = 45.3 Hz, 1H), 5.06 (dd, J = |
| | 5-yl)-3-methylbutanoyl)-4-hydroxy- | | 39.5, 3.2 Hz, 1H), 4.98-4.59 (m, 1H), 4.36 (t, J = |
| | N-((S)-1-(4-(4-methylthiazol-5- | | 7.8 Hz, 1H), 4.28 (s, 1H), 3.99 (s, 2H), 3.89 (s, |
| | yl)phenyl)ethyl)pyrrolidine-2- | | 2H), 3.81 (q, J = 7.8 Hz, 1H), 3.70 (dd, J = 10.5, |
| | carboxamide | | 4.4 Hz, 1H), 3.58 (d, J = 9.8 Hz, 1H), 3.43 (t, J = |
| | | | 10.9 Hz, 2H), 2.88-2.75 (m, 2H), 2.71 (s, 1H), |
| | | | 2.63 (s, 6H), 2.58-2.53 (m, 1H), 2.46 (d, J = 1.8 |
| | | | Hz, 3H), 2.37-2.13 (m, 1H), 2.02 (t, J = 9.5 Hz, |
| | | | 1H), 1.84-1.71 (m, 1H), 1.42 (dd, J = 29.7, 6.9 |
| | | | Hz, 3H), 0.95 (d, J = 6.5 Hz, 3H), 0.80 (dd, J = |
| | | | 14.9, 6.7 Hz, 3H). |
| 66 | (2S,4R)-4-hydroxy-1-((R)-2-(3-(6- | 841.35 | ¹H NMR (400 MHz, DMSO-d6) δ 14.45 (d, J = |
| | (3-(2-hydroxyphenyl)-5-(1- | | 80.3 Hz, 1H), 8.99 (d, J = 2.3 Hz, 1H), 8.57- |
| | methylcyclopropyl)-5H-pyrrolo[3,2- | | 8.30 (m, 2H), 8.18 (d, J = 7.8 Hz, 1H), 7.52- |
| | c]pyridazin-6-yl)-2- | | 7.24 (m, 5H), 7.03-6.89 (m, 3H), 5.96-5.72 |
| | azaspiro[3.3]heptan-2-yl)isoxazol- | | (m, 1H), 5.12 (d, J = 3.5 Hz, 1H), 5.04-4.77 (m, |
| | 5-yl)-3-methylbutanoyl)-N-((S)-1- | | 2H), 4.43-4.23 (m, 2H), 4.04 (d, J = 21.8 Hz, |
| | (4-(4-methylthiazol-5- | | 2H), 3.93-3.82 (m, 3H), 3.71 (dd, J = 10.6, 4.4 |
| | yl)phenyl)ethyl)pyrrolidine-2- | | Hz, 1H), 3.60 (d, J = 9.7 Hz, 1H), 3.48-3.42 (m, |
| | carboxamide | | 1H), 2.86-2.76 (m, 2H), 2.70-2.64 (m, 1H), |
| | | | 2.46 (d, J = 2.6 Hz, 3H), 2.27-2.16 (m, 1H), |
| | | | 2.10-1.98 (m, 1H), 1.84-1.72 (m, 1H), 1.49 |
| | | | (s, 3H), 1.38 (d, J = 7.0 Hz, 3H), 1.24 (d, J = 7.3 |
| | | | Hz, 2H), 1.15 (d, J = 7.5 Hz, 2H), 0.96 (dd, J = |
| | | | 8.4, 4.9 Hz, 3H), 0.81 (dd, J = 14.6, 6.7 Hz, 3H). |
| 67 | (2S,4R)-4-hydroxy-1-((R)-2-(3-(6- | 801.3 | ¹H NMR (300 MHz, DMSO-d6) δ 14.66 (s, 1H), |
| | (3-(2-hydroxyphenyl)-5-methyl-5H- | | 9.00 (d, J = 2.4 Hz, 1H), 8.58 (s, 1H), 8.42 (d, |
| | pyrrolo[3,2-c]pyridazin-6-yl)-2- | | J = 7.7 Hz, 1H), 8.16 (d, J = 7.2 Hz, 1H), 7.60- |
| | azaspiro[3.3]heptan-2-yl)isoxazol- | | 7.17 (m, 5H), 7.04-6.93 (m, 2H), 6.88 (s, 1H), |
| | 5-yl)-3-methylbutanoyl)-N-((S)-1- | | 5.88 (s, 1H), 5.12 (d, J = 3.6 Hz, 1H), 4.97-4.87 |
| | (4-(4-methylthiazol-5- | | (m, 1H), 4.37-4.29 (m, 2H), 4.04 (s, 2H), 3.86 |
| | yl)phenyl)ethyl)pyrrolidine-2- | | (s, 2H), 3.82-3.69 (m, 5H), 3.60 (d, J = 9.7 Hz, |
| | carboxamide | | 1H), 3.44 (s, 1H), 2.78 (dd, J = 19.6, 9.9 Hz, 3H), |
| | | | 2.47 (d, J = 1.9 Hz, 4H), 2.23 (s, 1H), 2.03 (t, |
| | | | J = 10.1 Hz, 1H), 1.86-1.73 (m, 1H), 1.42 (dd, J = |
| | | | 22.7, 7.0 Hz, 3H), 0.96 (d, J = 6.5 Hz, 3H), 0.82 |
| | | | (dd, J = 16.8, 6.2 Hz, 3H). |
| 68 | (2S,4R)-1-((R)-2-(3-(6-(5-(1- | 859.4 | ¹H NMR (400 MHz, DMSO-d6) δ 14.24 (s, 1H), |
| | (fluoromethyl)cyclopropyl)-3-(2- | | 8.99 (s, 1H), 8.42 (d, J = 7.7 Hz, 1H), 8.30 (s, |
| | hydroxyphenyl)-5H-pyrrolo[3,2- | | 1H), 8.15-8.02 (m, 1H), 7.52-7.27 (m, 5H), |
| | c]pyridazin-6-yl)-2- | | 7.07-6.92 (m, 3H), 5.89 (s, 1H), 5.12 (d, J = 3.6 |
| | azaspiro[3.3]heptan-2-yl)isoxazol- | | Hz, 1H), 5.05-4.84 (m, 1H), 4.68 (s, 1H), 4.55 |
| | 5-yl)-3-methylbutanoyl)-4-hydroxy- | | (d, J = 2.1 Hz, 1H), 4.37 (t, J = 7.9 Hz, 1H), 4.29 |
| | N-((S)-1-(4-(4-methylthiazol-5- | | (s, 1H), 4.05 (s, 2H), 3.90 (d, J = 21.3 Hz, 3H), |
| | yl)phenyl)ethyl)pyrrolidine-2- | | 3.71 (dd, J = 10.5, 4.4 Hz, 1H), 3.60 (d, J = 9.8 |
| | carboxamide | | Hz, 1H), 3.44 (dd, J = 12.2, 9.9 Hz, 1H), 2.87- |
| | | | 2.57 (m, 3H), 2.46 (s, 4H), 2.28-2.09 (m, 1H), |
| | | | 2.03 (t, J = 10.1 Hz, 1H), 1.79 (ddd, J = 12.7, 8.0, |
| | | | 4.7 Hz, 1H), 1.46 (d, J = 7.1 Hz, 3H), 1.38 (d, |
| | | | J = 7.0 Hz, 3H), 1.11 (s, 1H), 0.96 (t, J = 6.5 Hz, |
| | | | 3H), 0.81 (dd, J = 14.6, 6.6 Hz, 3H). |
| 77 | (2S,4R)-1-((R)-2-(3-(6-(5- | 841.3 | ¹H NMR (300 MHz, DMSO-d6) δ 14.54 (s, 1H), |
| | (cyclopropylmethyl)-3-(2- | | 8.99 (d, J = 1.7 Hz, 1H), 8.57 (s, 1H), 8.41 (d, |
| | hydroxyphenyl)-5H-pyrrolo[3,2- | | J = 7.6 Hz, 1H), 8.22-8.08 (m, 1H), 7.58-7.21 |
| | c]pyridazin-6-yl)-2- | | (m, 5H), 7.08-6.83 (m, 3H), 5.82 (d, J = 32.9 |
| | azaspiro[3.3]heptan-2-yl)isoxazol- | | Hz, 1H), 5.11 (d, J = 3.6 Hz, 1H), 4.92 (t, J = 7.2 |
| | 5-yl)-3-methylbutanoyl)-4-hydroxy- | | Hz, 1H), 4.37 (t, J = 7.8 Hz, 1H), 4.29 (s, 1H), |
| | N-((S)-1-(4-(4-methylthiazol-5- | | 4.15 (d, J = 6.9 Hz, 2H), 4.05 (s, 2H), 3.87 (s, |
| | yl)phenyl)ethyl)pyrrolidine-2- | | 2H), 3.82-3.64 (m, 2H), 3.59 (d, J = 9.7 Hz, |
| | carboxamide | | 1H), 3.42 (d, J = 10.6 Hz, 1H), 2.80 (t, J = 10.0 |
| | | | Hz, 2H), 2.57 (s, 2H), 2.46 (d, J = 1.8 Hz, 3H), |
| | | | 2.32-2.11 (m, 1H), 2.01 (d, J = 8.0 Hz, 1H), |
| | | | 1.84-1.67 (m, 1H), 1.42 (dd, J = 23.1, 7.0 Hz, |
| | | | 3H), 1.29-1.14 (m, 1H), 0.95 (d, J = 6.4 Hz, |
| | | | 3H), 0.79 (d, J = 6.7 Hz, 3H), 0.49 (dd, J = 6.6, |
| | | | 4.8 Hz, 4H). |
| 78 | (2S,4R)-1-((R)-2-(3-(6-(5- | 827.35 | ¹H NMR (300 MHz, DMSO-d6) δ 14.34 (s, 1H), |
| | cyclopropyl-3-(2-hydroxyphenyl)- | | 8.99 (d, J = 2.2 Hz, 1H), 8.41 (d, J = 7.7 Hz, 1H), |
| | 5H-pyrrolo[3,2-c]pyridazin-6-yl)-2- | | 8.30 (s, 1H), 8.18-8.09 (m, 1H), 7.44 (d, J = 8.3 |
| | azaspiro[3.3]heptan-2-yl)isoxazol- | | Hz, 2H), 7.40-7.26 (m, 3H), 7.04-6.93 (m, |
| | 5-yl)-3-methylbutanoyl)-4-hydroxy- | | 2H), 6.86 (s, 1H), 5.88 (s, 1H), 5.11 (d, J = 3.6 |
| | N-((S)-1-(4-(4-methylthiazol-5- | | Hz, 1H), 4.97-4.86 (m, 1H), 4.37 (t, J = 7.9 Hz, |
| | yl)phenyl)ethyl)pyrrolidine-2- | | 1H), 4.32-4.26 (m, 1H), 4.05 (s, 2H), 3.86 (s, |
| | carboxamide | | 3H), 3.76-3.65 (m, 1H), 3.59 (d, J = 9.8 Hz, |
| | | | 1H), 3.47-3.41 (m, 1H), 3.31-3.21 (m, 1H), |
| | | | 2.81 (t, J = 10.2 Hz, 2H), 2.58-2.52 (m, 2H), |
| | | | 2.46 (s, 3H), 2.26-2.12 (m, 1H), 2.11-1.97 (m, |
| | | | 1H), 1.86-1.71 (m, 1H), 1.42 (dd, J = 23.0, 7.0 |
| | | | Hz, 3H), 1.29-1.20 (m, 2H), 1.08 (d, J = 7.6 Hz, |
| | | | 2H), 0.95 (d, J = 6.5 Hz, 3H), 0.79 (d, J = 6.7 Hz, |
| | | | 3H). |
| 76 | (2S,4R)-1-((R)-2-(3-(4-(5- | 815.25 | ¹H NMR (400 MHz, DMSO-d6) δ 14.31 (s, 1H), |
| | cyclopropyl-3-(2-hydroxyphenyl)- | | 8.99 (s, 1H), 8.42 (d, J = 7.7 Hz, 1H), 8.35 (s, |
| | 5H-pyrrolo[3,2-c]pyridazin-6- | | 1H), 8.18-8.11 (m, 1H), 7.50-7.41 (m, 2H), |
| | yl)piperidin-1-yl)isoxazol-5-yl)-3- | | 7.41-7.34 (m, 2H), 7.37-7.28 (m, 1H), 7.03- |
| | methylbutanoyl)-4-hydroxy-N-((S)- | | 6.95 (m, 2H), 6.83-6.77 (m, 1H), 6.23 (s, 1H), |
| | 1-(4-(4-methylthiazol-5- | | 5.13 (d, J = 3.6 Hz, 1H), 4.98-4.86 (m, 1H), |
| | yl)phenyl)ethyl)pyrrolidine-2- | | 4.38 (t, J = 7.8 Hz, 1H), 4.30 (s, 1H), 3.84 (d, |
| | carboxamide | | J = 12.3 Hz, 2H), 3.73 (dd, J = 10.4, 4.4 Hz, 1H), |
| | | | 3.60 (d, J = 9.9 Hz, 1H), 3.74-3.21 (m, 3H), |
| | | | 2.99 (t, J = 12.1 Hz, 2H), 2.48-2.44 (m, 3H), |
| | | | 2.20 (d, J = 12.6 Hz, 1H),2.05- |
| | | | 2.20(m,2H), 2.00(s, 1H), 1.85-1.72 (m, 3H), 1.47 |
| | | | (d, J = 6.9 Hz, 3H), 1.42-1.21 (m, 2H), 1.19- |
| | | | 1.12 (m, 2H), 1.02-0.94 (m, 3H), 0.89-0.79 |
| | | | (m, 3H). |
| 79 | (2S,4R)-1-((R)-2-(3-((S)-3-(5- | 815.3 | ¹H NMR (400 MHz, DMSO-d6) δ 14.03 (d, J = |
| | cyclobutyl-3-(2-hydroxyphenyl)-5H- | | 8.6 Hz, 1H), 8.99 (s, 1H), 8.47 (s, 1H), 8.41 (d, |
| | pyrrolo[3,2-c]pyridazin-6- | | J = 7.6 Hz, 1H), 8.18 (d, J = 8.1 Hz, 1H), 7.44 (d, |
| | yl)pyrrolidin-1-yl)isoxazol-5-yl)-3- | | J = 8.4 Hz, 2H), 7.40-7.29 (m, 3H), 7.04-6.95 |
| | methylbutanoyl)-4-hydroxy-N-((S)- | | (m, 2H), 6.87 (s, 1H), 6.04 (s, 1H), 5.25-5.16 |
| | 1-(4-(4-methylthiazol-5- | | (m, 1H), 5.11 (d, J = 3.7 Hz, 1H), 4.96-4.88 (m, |
| | yl)phenyl)ethyl)pyrrolidine-2- | | 1H), 4.41-4.33 (m, 1H), 4.29 (s, 1H), 3.97- |
| | carboxamide | | 3.89 (m, 1H), 3.87-3.78 (m, 1H), 3.76-3.69 |
| | | | (m, 1H), 3.59 (d, J = 9.9 Hz, 1H), 3.49 (s, 1H), |
| | | | 3.47-3.39 (m, 3H), 3.03-2.91 (m, 2H), 2.67 |
| | | | (s, 2H), 2.76-2.46 (m, 3H), 2.41-2.19 (m, 3H), |
| | | | 2.14-2.00 (m, 2H), 1.95-1.78 (m, 1H), 1.76- |
| | | | 1.52 (m, 1H), 1.38 (d, J = 7.0 Hz, 3H), 1.01- |
| | | | 0.93 (m, 3H), 0.88-0.79 (m, 3H). |
| 80 | (2S,4R)-4-hydroxy-1-((R)-2-(3-((S)- | 815.35 | ¹H NMR (400 MHz, DMSO-d6) δ 14.25 (s, 1H), |
| | 3-(3-(2-hydroxyphenyl)-5-(1- | | 8.99 (d, J = 2.0 Hz, 1H), 8.41 (d, J = 7.8 Hz, 2H), |
| | methylcyclopropyl)-5H-pyrrolo[3,2- | | 8.19 (dd, J = 8.1, 1.6 Hz, 1H), 7.57-7.20 (m, |
| | c]pyridazin-6-yl)pyrrolidin-1- | | 5H), 7.13-6.75 (m, 3H), 6.04 (s, 1H), 5.19- |
| | yl)isoxazol-5-yl)-3-methylbutanoyl)- | | 4.99 (m, 1H), 4.91 (q, J = 7.1 Hz, 1H), 4.38 (t, |
| | N-((S)-1-(4-(4-methylthiazol-5- | | J = 7.8 Hz, 1H), 4.30 (s, 1H), 4.08 (p, J = 7.9 Hz, |
| | yl)phenyl)ethyl)pyrrolidine-2- | | 1H), 3.88 (dd, J = 17.6, 8.5 Hz, 1H), 3.73 (dd, J = |
| | carboxamide | | 10.5, 4.5 Hz, 1H), 3.65-3.37 (m, 6H), 2.46 (d, |
| | | | J = 2.2 Hz, 3H), 2.26 (q, J = 10.0, 8.4 Hz, 2H), |
| | | | 2.08-1.97 (m, 1H), 1.79 (ddd, J = 12.7, 8.0, 4.8 |
| | | | Hz, 1H), 1.58 (d, J = 3.1 Hz, 3H), 1.49-1.35 (m, |
| | | | 3H), 1.31 (s, 2H), 1.21 (d, J = 19.5 Hz, 2H), 0.98 |
| | | | (t, J = 7.4 Hz, 3H), 0.85 (dd, J = 15.4, 6.7 Hz, |
| | | | 3H). |
| 81 | (2S,4R)-4-hydroxy-1-((R)-2-(3- | 815.25 | ¹H NMR (400 MHz, Methanol-d4) δ 8.87 (d, J = |
| | ((R)-3-(3-(2-hydroxyphenyl)-5-(1- | | 4.5 Hz, 1H), 8.67-8.57 (m, 1H), 8.36 (s, 1H), |
| | methylcyclopropyl)-5H-pyrrolo[3,2- | | 8.01 (dd, J = 8.1, 1.6 Hz, 1H), 7.48-7.35 (m, |
| | c]pyridazin-6-yl)pyrrolidin-1- | | 4H), 7.37-7.28 (m, 1H), 7.06-6.96 (m, 2H), |
| | yl)isoxazol-5-yl)-3-methylbutanoyl)- | | 6.83-6.74 (m, 2H), 6.06 (s, 1H), 5.09-4.99 (m, |
| | N-((S)-1-(4-(4-methylthiazol-5- | | 2H), 4.58-4.49 (m, 1H), 4.43 (d, J = 16.1 Hz, |
| | yl)phenyl)ethyl)pyrrolidine-2- | | 1H), 4.26-4.16 (m, 1H), 4.00-3.89 (m, 2H), |
| | carboxamide | | 3.82-3.47 (m, 5H), 2.64 (s, 1H), 2.48 (s, 3H), |
| | | | 2.43-2.30 (m, 2H), 2.24-2.15 (m, 1H), 2.02- |
| | | | 1.91 (m, 1H), 1.68-1.57 (m, 3H), 1.53 (d, J = |
| | | | 7.0 Hz, 3H), 1.40-1.27 (m, 4H), 1.07 (d, J = 6.6 |
| | | | Hz, 3H), 0.98-0.89 (m, 3H). |
| 84 | (2S,4R)-1-((R)-2-(3-(2-(5- | 841.35 | ¹H NMR (300 MHz, DMSO-d6) δ 14.38 (s, 1H), |
| | cyclopropyl-3-(2-hydroxyphenyl)- | | 8.99 (s, 1H), 8.52 (t, J = 5.9 Hz, 1H), 8.29 (s, |
| | 5H-pyrrolo[3,2-c]pyridazin-6-yl)-7- | | 1H), 8.13 (d, J = 8.5 Hz, 1H), 7.55-7.19 (m, |
| | azaspiro[3.5]nonan-7-yl)isoxazol- | | 5H), 7.10-6.91 (m, 2H), 6.85 (d, J = 4.2 Hz, |
| | 5-yl)-3-methylbutanoyl)-4-hydroxy- | | 1H), 6.17 (s, 1H), 5.13 (d, J = 3.7 Hz, 1H), 4.71- |
| | N-(4-(4-methylthiazol-5- | | 4.20 (m, 4H), 3.96 (q, J = 8.5 Hz, 1H), 3.79 |
| | yl)benzyl)pyrrolidine-2- | | (dd, J = 10.4, 4.4 Hz, 1H), 3.59 (d, J = 9.7 Hz, |
| | carboxamide | | 1H), 3.43 (d, J = 10.9 Hz, 1H), 3.28-3.17 (m, |
| | | | 3H), 3.10 (s, 2H), 2.50-2.34 (m, 5H), 2.32- |
| | | | 2.18 (m, 1H), 2.17-1.96 (m, 3H), 1.95-1.82 |
| | | | (m, 1H), 1.81 (s, 2H), 1.64 (s, 2H), 1.25 (d, J = |
| | | | 7.6 Hz, 2H), 1.08 (s, 2H), 0.95 (d, J = 6.5 Hz, |
| | | | 3H), 0.81 (d, J = 6.0 Hz, 3H). |
| 85 | (2S,4R)-1-((R)-2-(3-(9-(5- | 883.4 | ¹H NMR (400 MHz, DMSO-d6) δ 14.37 (s, 1H), |
| | cyclopropyl-3-(2-hydroxyphenyl)- | | 8.99 (d, J = 2.3 Hz, 1H), 8.41 (d, J = 7.7 Hz, 1H), |
| | 5H-pyrrolo[3,2-c]pyridazin-6-yl)-3- | | 8.32 (s, 1H), 8.17-8.10 (m, 1H), 7.50-7.41 (m, |
| | azaspiro[5.5]undecan-3- | | 2H), 7.38-7.27 (m, 3H), 6.99 (dd, J = 8.1, 6.9 |
| | yl)isoxazol-5-yl)-3-methylbutanoyl)- | | Hz, 2H), 6.83 (s, 1H), 6.15 (s, 1H), 5.12 (d, J = |
| | 4-hydroxy-N-((S)-1-(4-(4- | | 3.7 Hz, 1H), 4.93 (q, J = 7.2, 6.4 Hz, 1H), 4.37 |
| | methylthiazol-5- | | (t, J = 7.8 Hz, 1H), 4.29 (s, 1H), 3.72 (dd, J = |
| | yl)phenyl)ethyl)pyrrolidine-2- | | 10.5, 4.5 Hz, 1H), 3.57 (d, J = 9.9 Hz, 1H), 3.38- |
| | carboxamide | | 3.27 (m, 3H), 3.21 (d, J = 5.6 Hz, 5H), 2.46 (s, |
| | | | 3H), 2.29-2.17 (m, 1H), 2.01-1.84 (m, 5H), |
| | | | 1.83-1.68 (m, 4H), 1.46 (s, 3H), 1.38 (d, J = 7.1 |
| | | | Hz, 3H), 1.30 (d, J = 6.3 Hz, 3H), 1.11 (s, 2H), |
| | | | 0.97 (t, J = 7.0 Hz, 3H), 0.82 (dd, J = 15.7, 6.7 |
| | | | Hz, 3H). |
| 86 | (2S,4R)-1-((R)-2-(3-(2-(5- | 869.35 | ¹H NMR (300 MHz, DMSO-d6) δ 14.59 (s, 1H), |
| | (cyclopropylmethyl)-3-(2- | | 9.00 (s, 1H), 8.57 (s, 1H), 8.42 (d, J = 7.6 Hz, |
| | hydroxyphenyl)-5H-pyrrolo[3,2- | | 1H), 8.17 (dd, J = 8.5, 1.6 Hz, 1H), 7.59-7.21 |
| | c]pyridazin-6-yl)-7- | | (m, 5H), 7.08-6.85 (m, 3H), 6.11 (d, J = 40.2 |
| | azaspiro[3.5]nonan-7-yl)isoxazol- | | Hz, 1H), 5.12 (d, J = 3.6 Hz, 1H), 4.92 (t, J = 7.2 |
| | 5-yl)-3-methylbutanoyl)-4-hydroxy- | | Hz, 1H), 4.37 (t, J = 7.8 Hz, 1H), 4.29 (s, 1H), |
| | N-((S)-1-(4-(4-methylthiazol-5- | | 4.17 (d, J = 7.0 Hz, 2H), 3.90 (q, J = 8.8 Hz, 1H), |
| | yl)phenyl)ethyl)pyrrolidine-2- | | 3.72 (dd, J = 10.5, 4.5 Hz, 1H), 3.58 (d, J = 9.8 |
| | carboxamide | | Hz, 1H), 3.43 (d, J = 10.4 Hz, 1H), 3.10 (d, J = |
| | | | 13.4 Hz, 2H), 2.46 (s, 4H), 2.32-1.97 (m, 5H), |
| | | | 1.81 (d, J = 12.1 Hz, 3H), 1.64 (s, 2H), 1.43 (dd, |
| | | | J = 23.7, 7.0 Hz, 3H), 1.22 (d, J = 11.8 Hz, 1H), |
| | | | 0.96 (d, J = 6.5 Hz, 3H), 0.82 (dd, J = 12.1, 6.7 |
| | | | Hz, 3H), 0.58-0.39 (m, 4H). |
| 113 | (2S,4R)-4-hydroxy-1-((R)-2-(3-(2- | 829.4 | ¹H NMR (300 MHz, DMSO-d6) δ 14.68 (s, 1H), |
| | (3-(2-hydroxyphenyl)-5-methyl-5H- | | 8.99 (s, 1H), 8.56 (s, 1H), 8.41 (d, J = 7.8 Hz, |
| | pyrrolo[3,2-c]pyridazin-6-yl)-7- | | 1H), 8.16 (d, J = 7.1 Hz, 1H), 7.44 (d, J = 8.1 Hz, |
| | azaspiro[3.5]nonan-7-yl)isoxazol- | | 2H), 7.37 (d, J = 8.3 Hz, 3H), 7.03-6.93 (m, |
| | 5-yl)-3-methylbutanoyl)-N-((S)-1- | | 2H), 6.86 (s, 1H), 6.16 (s, 1H), 5.11 (d, J = 3.7 |
| | (4-(4-methylthiazol-5- | | Hz, 1H), 4.92 (t, J = 7.1 Hz, 1H), 4.37 (t, J = 7.9 |
| | yl)phenyl)ethyl)pyrrolidine-2- | | Hz, 2H), 3.87 (t, J = 8.6 Hz, 1H), 3.82-3.77 (m, |
| | carboxamide | | 3H), 3.75-3.68 (m, 1H), 3.62-3.48 (m, 2H), |
| | | | 3.39-3.31 (m, 1H), 3.19-3.11 (m, 2H), 3.11 |
| | | | (s, 2H), 2.49-2.37 (m, 3H), 2.37-2.34 (m, 1H), |
| | | | 2.31-2.21(m, 1H), 2.18-1.99(m, 3H), 1.86- |
| | | | 1.81 (m, 3H), 1.68-1.61 (m, 2H), 1.42 (dd, J = |
| | | | 23.3, 7.0 Hz, 3H), 0.95 (d, J = 6.4 Hz, 3H), 0.82 |
| | | | (dd, J = 11.9, 6.6 Hz, 3H). |
| 117 | (2S,4R)-1-((R)-2-(3-(2-(5- | 855.40 | ¹H NMR (300 MHz, DMSO-d6) δ 14.39 (s, 1H), |
| | cyclopropyl-3-(2-hydroxyphenyl)- | | 8.99 (d, J = 1.8 Hz, 1H), 8.42 (d, J = 7.7 Hz, 1H), |
| | 5H-pyrrolo[3,2-c]pyridazin-6-yl)-7- | | 8.29 (s, 1H), 8.13 (dd, J = 8.5, 1.6 Hz, 1H), 7.53- |
| | azaspiro[3.5]nonan-7-yl)isoxazol- | | 7.26 (m, 5H), 7.04-6.93 (m, 2H), 6.86 (s, 1H), |
| | 5-yl)-3-methylbutanoyl)-4-hydroxy- | | 6.17 (s, 1H), 5.20-4.68 (m, 2H), 4.37 (t, J = 7.8 |
| | N-((S)-1-(4-(4-methylthiazol-5- | | Hz, 1H), 4.29 (s, 1H), 3.98 (p, J = 8.8 Hz, 1H), |
| | yl)phenyl)ethyl)pyrrolidine-2- | | 3.72 (dd, J = 10.5, 4.4 Hz, 1H), 3.57 (d, J = 9.8 |
| | carboxamide | | Hz, 1H), 3.43 (d, J = 10.3 Hz, 1H), 3.30-3.22 |
| | | | (m, 3H), 3.12 (s, 2H), 2.48-2.36 (m, 5H), 2.30- |
| | | | 2.18 (m, 1H), 2.17-2.11 (m, 2H), 2.08-1.98 |
| | | | (m, 1H), 1.85-1.70 (m, 3H), 1.62 (s, 2H), 1.42 |
| | | | (dd, J = 23.6, 7.0 Hz, 3H), 1.25 (d, J = 7.1 Hz, |
| | | | 2H), 1.14-1.05 (m, 2H), 0.96 (t, J = 6.0 Hz, 3H), |
| | | | 0.82 (dd, J = 12.2, 6.6 Hz, 3H). |
| 88 | (2S,4R)-1-((R)-2-(3-((R)-3-(5- | 815.35 | ¹H NMR (400 MHz, DMSO-d6) δ 14.07-14.00 |
| | cyclobutyl-3-(2-hydroxyphenyl)-5H- | | (m, 1H), 9.01-8.96 (m, 1H), 8.47 (s, 1H), 8.41 |
| | pyrrolo[3,2-c]pyridazin-6- | | (d, J = 7.7 Hz, 1H), 8.18 (d, J = 7.9 Hz, 1H), 7.49- |
| | yl)pyrrolidin-1-yl)isoxazol-5-yl)-3- | | 7.41 (m, 2H), 7.40-7.29 (m, 3H), 7.04-6.96 |
| | methylbutanoyl)-4-hydroxy-N-((S)- | | (m, 2H), 6.86 (s, 1H), 6.04 (s, 1H), 5.25-5.14 |
| | 1-(4-(4-methylthiazol-5- | | (m, 1H), 5.11 (d, J = 3.6 Hz, 1H), 4.96-4.88 (m, |
| | yl)phenyl)ethyl)pyrrolidine-2- | | 1H), 4.42-4.33 (m, 1H), 4.32-4.27 (m, 1H), |
| | carboxamide | | 3.97-3.89 (m, 1H), 3.85-3.77 (m, 1H), 3.77- |
| | | | 3.70 (m, 1H), 3.59 (d, J = 9.8 Hz, 1H), 3.50- |
| | | | 3.40 (m, 4H), 3.02-2.91 (m, 2H), 2.58-2.53 |
| | | | (m, 2H), 2.48-2.44 (m, 3H), 2.28-2.15 (m, |
| | | | 2H), 2.14-1.98 (m, 2H), 1.95-1.84 (m, 1H), |
| | | | 1.84-1.73 (m, 2H), 1.38 (d, J = 7.0 Hz, 3H), |
| | | | 1.01-0.93 (m, 3H), 0.88-0.78 (m, 3H). |
| 99 | (2S,4R)-1-((R)-2-(3-((S)-3-(5- | 801.15 | ¹H NMR (400 MHz, Methanol-d4) δ 8.87 (d, J = |
| | cyclopropyl-3-(2-hydroxyphenyl)- | | 5.7 Hz, 1H), 8.36-8.31 (m, 1H), 8.01 (d, J = 7.9 |
| | 5H-pyrrolo[3,2-c]pyridazin-6- | | Hz, 1H), 7.48-7.36 (m, 4H), 7.38-7.27 (m, |
| | yl)pyrrolidin-1-yl)isoxazol-5-yl)-3- | | 1H), 7.00 (t, J = 8.8 Hz, 2H), 6.76 (d, J = 14.0 |
| | methylbutanoyl)-4-hydroxy-N-((S)- | | Hz, 1H), 6.06 (s, 1H), 5.04 (d, J = 7.0 Hz, 1H), |
| | 1-(4-(4-methylthiazol-5- | | 4.52 (t, J = 8.2 Hz, 1H), 4.45 (s, 1H), 4.21-4.12 |
| | yl)phenyl)ethyl)pyrrolidine-2- | | (m, 1H), 4.02-3.91 (m, 1H), 3.85 (dd, J = 10.9, |
| | carboxamide | | 4.2 Hz, 1H), 3.69-3.49 (m, 5H), 3.46-3.41 (m, |
| | | | 1H), 2.64 (dt, J = 12.0, 5.4 Hz, 1H), 2.48 (s, 3H), |
| | | | 2.44-2.31 (m, 2H), 2.24-2.14 (m, 1H), 2.05- |
| | | | 1.91 (m, 1H), 1.59 (d, J = 7.1 Hz, 3H), 1.43- |
| | | | 1.37 (m, 2H), 1.28 (s, 2H), 1.06 (d, J = 6.6 Hz, |
| | | | 3H), 0.93 (dd, J = 10.7, 6.7 Hz, 3H). |
| 87 | (2S,4R)-1-((R)-2-(3-(7-(5- | 855.35 | ¹H NMR (400 MHz, DMSO-d6) δ 14.34 (s, 1H), |
| | cyclopropyl-3-(2-hydroxyphenyl)- | | 8.99 (d, J = 2.2 Hz, 1H), 8.42 (d, J = 7.6 Hz, 1H), |
| | 5H-pyrrolo[3,2-c]pyridazin-6-yl)-2- | | 8.33 (s, 1H), 8.15-8.08 (m, 1H), 7.49-7.42 (m, |
| | azaspiro[3.5]nonan-2-yl)isoxazol- | | 2H), 7.40-7.28 (m, 3H), 6.99 (dd, J = 8.1, 6.9 |
| | 5-yl)-3-methylbutanoyl)-4-hydroxy- | | Hz, 2H), 6.75 (s, 1H), 5.87 (s, 1H), 5.12 (d, J = |
| | N-((S)-1-(4-(4-methylthiazol-5- | | 3.4 Hz, 1H), 4.93 (q, J = 7.1 Hz, 1H), 4.37 (t, J = |
| | yl)phenyl)ethyl)pyrrolidine-2- | | 7.8 Hz, 1H), 4.29 (s, 1H), 3.72 (s, 3H), 3.59 (d, |
| | carboxamide | | J = 9.2 Hz, 3H), 3.50-3.37 (m, 2H), 3.10 (d, J = |
| | | | 21.5 Hz, 1H), 2.46 (d, J = 1.7 Hz, 3H), 2.22 (q, |
| | | | J = 7.7, 7.2 Hz, 1H), 2.07 (d, J = 15.4 Hz, 5H), |
| | | | 1.84-1.63 (m, 3H), 1.61-1.42 (m, 2H), 1.39 (d, J = |
| | | | 7.0 Hz, 3H), 1.34-1.27 (m, 2H), 1.12 (s, 2H), |
| | | | 0.96 (d, J = 6.5 Hz, 3H), 0.80 (d, J = 6.7 Hz, 3H). |
| 96 | (2S,4R)-1-((R)-2-(3-((S)-2-(5- | 869.2 | ¹H NMR (400 MHz, Methanol-d4) δ 8.86 (d, J = |
| | cyclopropyl-3-(2-hydroxyphenyl)- | | 6.8 Hz, 1H), 8.29 (s, 1H), 7.99 (d, J = 7.9 Hz, |
| | 5H-pyrrolo[3,2-c]pyridazin-6-yl)-8- | | 1H), 7.48-7.35 (m, 4H), 7.31 (td, J = 7.7, 1.6 |
| | azaspiro[4.5]decan-8-yl)isoxazol-5- | | Hz, 1H), 7.00 (t, J = 7.9 Hz, 2H), 6.71 (s, 1H), |
| | yl)-3-methylbutanoyl)-4-hydroxy-N- | | 6.12 (s, 1H), 5.03 (d, J = 7.0 Hz, 1H), 4.62-4.37 |
| | ((S)-1-(4-(4-methylthiazol-5- | | (m, 2H), 3.84 (dd, J = 10.9, 4.4 Hz, 2H), 3.68- |
| | yl)phenyl)ethyl)pyrrolidine-2- | | 3.53 (m, 2H), 3.48-3.44 (m, 1H), 3.42-3.38 |
| | carboxamide | | (m, 2H), 3.30 (s, 1H), 2.48 (s, 3H), 2.40-2.32 |
| | | | (m, 3H), 2.23-2.13 (m, 1H), 1.98 (dt, J = 18.3, |
| | | | 9.0 Hz, 4H), 1.83-1.66 (m, 6H), 1.52 (d, J = |
| | | | 7.0 Hz, 3H), 1.36 (d, J = 6.8 Hz, 2H), 1.22-1.10 |
| | | | (m, 2H), 1.05 (d, J = 6.7 Hz, 3H), 0.91 (dd, J = |
| | | | 12.1, 6.6 Hz, 3H). |
| 97 | (2S,4R)-1-((R)-2-(3-((R)-2-(5- | 869.2 | ¹H NMR (300 MHz, DMSO-d6) δ 14.38 (s, 1H), |
| | cyclopropyl-3-(2-hydroxyphenyl)- | | 8.99 (s, 1H), 8.41 (d, J = 7.7 Hz, 1H), 8.31 (s, |
| | 5H-pyrrolo[3,2-c]pyridazin-6-yl)-8- | | 1H), 8.14 (d, J = 7.3 Hz, 1H), 7.45 (d, J = 8.2 Hz, |
| | azaspiro[4.5]decan-8-yl)isoxazol-5- | | 2H), 7.41-7.27 (m, 3H), 6.99 (dd, J = 8.0, 6.6 |
| | yl)-3-methylbutanoyl)-4-hydroxy-N- | | Hz, 2H), 6.82 (s, 1H), 6.16 (s, 1H), 5.12 (d, J = |
| | ((S)-1-(4-(4-methylthiazol-5- | | 3.6 Hz, 1H), 4.92 (t, J = 7.4 Hz, 1H), 4.37 (t, J = |
| | yl)phenyl)ethyl)pyrrolidine-2- | | 7.9 Hz, 1H), 4.29 (s, 1H), 3.71-3.65 (m, 2H), |
| | carboxamide | | 3.58 (d, J = 9.8 Hz, 1H), 3.39-3.33 (m, 2H), |
| | | | 3.24 (d, J = 13.7 Hz, 4H), 2.46-2.37 (m, 3H), |
| | | | 2.23-2.18 (m, 3H), 2.06-1.87 (m, 2H), 1.79- |
| | | | 1.71 (m, 2H), 1.64 (s, 6H), 1.43 (dd, J = 23.6, 6.9 |
| | | | Hz, 3H), 1.34-1.17 (m, 2H), 1.15-1.11 (m, |
| | | | 2H), 0.96 (d, J = 6.4 Hz, 3H), 0.80 (d, J = 6.7 Hz, |
| | | | 3H). |
| 98 | (2S,4R)-1-((R)-2-(3-((S)-3-(5- | 815.2 | ¹H NMR (400 MHz, DMSO-d6) δ 14.27 (s, 1H), |
| | cyclopropyl-3-(2-hydroxyphenyl)- | | 8.98 (s, 1H), 8.40 (d, J = 7.7 Hz, 1H), 8.35 (d, |
| | 5H-pyrrolo[3,2-c]pyridazin-6- | | J = 3.0 Hz, 1H), 8.18-8.11 (m, 1H), 7.48-7.40 |
| | yl)piperidin-1-yl)isoxazol-5-yl)-3- | | (m, 2H), 7.37-7.28 (m, 1H), 7.36 (d, J = 8.1 Hz, |
| | methylbutanoyl)-4-hydroxy-N-((S)- | | 2H), 6.99 (t, J = 7.6 Hz, 2H), 6.88 (s, 1H), 6.28 |
| | 1-(4-(4-methylthiazol-5- | | (s, 1H), 5.12 (d, J = 3.6 Hz, 1H), 4.91 (p, J = 7.0 |
| | yl)phenyl)ethyl)pyrrolidine-2- | | Hz, 1H), 4.37 (t, J = 7.8 Hz, 1H), 4.29 (s, 1H), |
| | carboxamide | | 3.98 (d, J = 12.5 Hz, 1H), 3.80-3.68 (m, 2H), |
| | | | 3.60 (d, J = 9.8 Hz, 1H), 3.51-3.40 (m, 3H), |
| | | | 3.07 (t, J = 11.7 Hz, 1H), 2.98 (t, J = 11.9 Hz, |
| | | | 1H), 2.45 (d, J = 3.4 Hz, 3H), 2.33-2.13 (m, |
| | | | 2H), 2.02 (t, J = 8.6 Hz, 1H), 1.78 (h, J = 12.4 |
| | | | Hz, 4H), 1.48-1.34 (m, 3H), 1.32-1.21 (m, |
| | | | 2H), 1.13 (dd, J = 19.4, 4.8 Hz, 2H), 0.97 (d, J = |
| | | | 6.6 Hz, 3H), 0.84 (dd, J = 16.5, 6.7 Hz, 3H). |
| 100 | (2S,4R)-1-((R)-2-(3-((R)-3-(5- | 815.3 | ¹H NMR (300 MHz, DMSO-d6) δ 14.34 (s, 1H), |
| | cyclopropyl-3-(2-hydroxyphenyl)- | | 9.08 (s, 1H), 8.45-8.33 (m, 2H), 8.20 (d, J = 7.7 |
| | 5H-pyrrolo[3,2-c]pyridazin-6- | | Hz, 1H), 7.48-7.42 (m, 2H), 7.39-7.28 (m, 3H), |
| | yl)piperidin-1-yl)isoxazol-5-yl)-3- | | 7.04-6.96 (m, 2H), 6.92 (s, 1H), 6.36 (s, 1H), |
| | methylbutanoyl)-4-hydroxy-N-((S)- | | 5.17 (d, J = 3.8 Hz, 1H), 5.01-4.88 (m, 1H), 4.42- |
| | 1-(4-(4-methylthiazol-5- | | 4.24 (m, 2H), 4.06-3.91 (m, 1H), 3.84-3.67 (m, |
| | yl)phenyl)ethyl)pyrrolidine-2- | | 2H), 3.64 (d, J = 9.8 Hz, 1H), 3.53-3.40 (m, 3H), |
| | carboxamide | | 3.10-2.90 (m, 2H), 2.46 (s, 3H), 2.32-2.11 (m, |
| | | | 2H), 2.09-2.00 (m, 1H), 1.88-1.66 (m, 3H), 1.49- |
| | | | 1.36 (m, 3H), 1.34-1.26 (m, 2H), 1.25-1.05 (m, |
| | | | 3H), 1.02-0.92 (m, 3H), 0.84-0.78 (m, 3H). |
| 101 | (2S,4R)-1-((R)-2-(3-((1S,5S)-6-(5- | 813.2 | ¹H NMR (300 MHz, DMSO-d6) δ 14.36 (s, 1H), |
| | cyclopropyl-3-(2-hydroxyphenyl)- | | 8.99 (s, 1H), 8.42 (d, J = 7.7 Hz, 1H), 8.31 (s, |
| | 5H-pyrrolo[3,2-c]pyridazin-6-yl)-3- | | 1H), 8.19-8.09 (m, 1H), 7.52-7.27 (m, 5H), |
| | azabicyclo[3.1.0]hexan-3- | | 6.99 (t, J = 7.5 Hz, 2H), 6.71 (s, 1H), 6.06 (s, |
| | yl)isoxazol-5-yl)-3-methylbutanoyl)- | | 1H), 5.13 (d, J = 3.5 Hz, 1H), 4.93 (t, J = 7.2 Hz, |
| | 4-hydroxy-N-((S)-1-(4-(4- | | 1H), 4.38 (t, J = 7.8 Hz, 1H), 4.30 (s, 1H), 3.92- |
| | methylthiazol-5- | | 3.54 (m, 4H), 3.44 (d, J = 8.7 Hz, 4H), 2.47 (s, |
| | yl)phenyl)ethyl)pyrrolidine-2- | | 3H), 2.34 (s, 2H), 2.28 (d, J = 3.8 Hz, 2H), 2.02 |
| | carboxamide | | (d, J = 9.6 Hz, 1H), 1.85-1.69 (m, 1H), 1.52- |
| | | | 1.36 (m, 3H), 1.33-1.26 (m, 2H), 1.14 (d, J = |
| | | | 3.7 Hz, 2H), 0.98 (d, J = 6.4 Hz, 3H), 0.85 (dd, |
| | | | J = 11.6, 5.5 Hz, 3H). |

### Preparation of (2S,4R)-1-[2-(3-{6-[5-cyclobutyl-3-(2-hydroxyphenyl)pyrrolo[3,2-c]pyridazin-6-yl]-2-azaspiro[3.3]heptan-2-yl}-1,2-oxazol-5-yl)-3-methylbutanoyl]-4-hydroxy-N-[(1S)-1-phenylethyl]pyrrolidine-2-carboxamide (Compound 49)

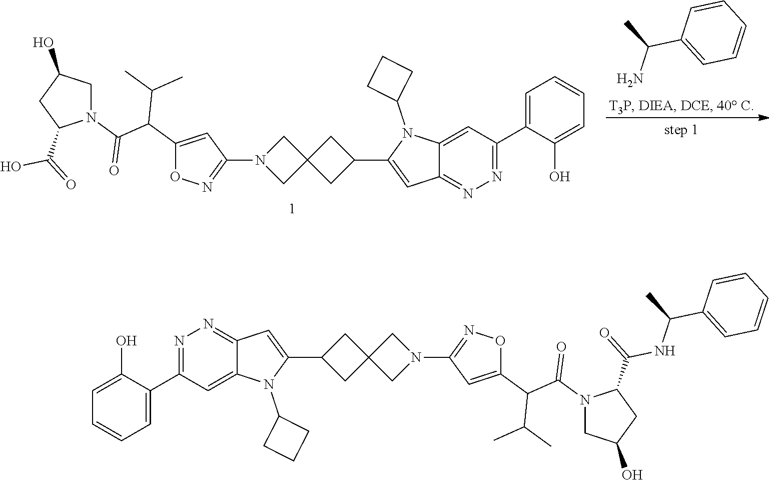

### Step 1: Preparation of (2S,4R)-1-[2-(3-{6-[5-cyclobutyl-3-(2-hydroxyphenyl)pyrrolo[3,2-c]pyridazin-6-yl]-2-azaspiro[3.3]heptan-2-yl}-1,2-oxazol-5-yl)-3-methylbutanoyl]-4-hydroxy-N-[(1S)-1-phenylethyl]pyrrolidine-2-carboxamide (Compound 49)

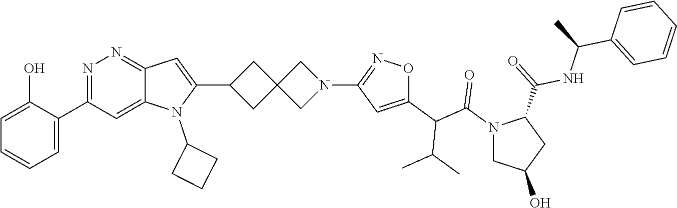 [SMILES: CC(C)C(C(=O)N1C[C@H](O)C[C@H]1C(=O)N[C@@H](C)c1ccccc1)c1cc(N2CC3(CC(c4cc5nnc(-c6ccccc6O)cc5n4C4CCC4)C3)C2)no1]

To a stirred solution of (2S,4R)-1-[2-(3-{6-[5-cyclobutyl-3-(2-hydroxyphenyl)pyrrolo[3,2-c]pyridazin-6-yl]-2-azaspiro[3.3]heptan-2-yl}-1,2-oxazol-5-yl)-3-methylbutanoyl]-4-hydroxypyrrolidine-2-carboxylic acid (40 mg, 0.062 mmol, 1 equiv), (S)-α-phenylethylamine (15.13 mg, 0.124 mmol, 2 equiv) and T₃P (59.59 mg, 0.186 mmol, 3 equiv) in DCE (3 mL) was added DIEA (24.21 mg, 0.186 mmol, 3 equiv). The resulting solution was stirred at 40° C. for 8 h. Desired product could be detected by LCMS. The resulting mixture was concentrated under reduced pressure. The crude product was purified by Prep-HPLC with the following conditions (Column: XBridge Prep OBD C18 Column, 30*150 mm, 10 μm; Mobile Phase A: Water (10 mmol/L NH₄HCO₃+0.05% NH₃H₂O), Mobile Phase B: ACN; Flow rate: 60 mL/min mL/min; Gradient: 36% B to 56% B in 8 min; Wave Length: 254 nm/220 nm nm; RT1(min): 9.95) to afford compound 49 (14.8 mg, 30.21%) as a white solid. ¹H NMR (300 MHz, DMSO-d6) δ 14.16 (d, J=3.1 Hz, 1H), 8.42 (s, 1H), 8.33 (d, J=7.9 Hz, 2H), 7.38-7.17 (m, 6H), 6.99 (t, J=7.4 Hz, 2H), 6.87 (s, 1H), 5.92-5.83 (m, 1H), 5.08 (d, J=3.6 Hz, 1H), 4.91 (dt, J=19.6, 9.7 Hz, 2H), 4.45-4.30 (m, 2H), 4.27 (s, 2H), 3.95 (d, J=4.9 Hz, 2H), 3.87-3.64 (m, 2H), 3.66-3.35 (m, 4H), 2.90 (d, J=10.6 Hz, 5H), 2.46 (t, J=11.8 Hz, 1H), 2.19-1.81 (m, 5H), 1.46-1.29 (m, 3H), 0.95 (d, J=6.4 Hz, 3H), 0.86-0.71 (m, 3H). LCMS (ESI) m/z: [M+H]⁺=744.35.

Compounds in Table 13 were prepared using procedures similar to the one used above for the preparation of Compound 49 using the appropriate amines.

TABLE 13

| | | LCMS (ESI) | |
|---|---|---|---|
| No. | Name | m/z | ¹H NMR |
| 50 | (2S,4R)-1-(2-(3-(6-(5-cyclobutyl-3- | 748.30 | ¹H NMR (400 MHz, DMSO-d6) δ 14.16 (d, J = |
| | (2-hydroxyphenyl)-5H-pyrrolo[3,2- | | 3.0 Hz, 1H), 8.51-8.37 (m, 2H), 8.17 (d, |
| | c]pyridazin-6-yl)-2- | | J = 8.1 Hz, 1H), 7.40-7.23 (m, 3H), 7.20- |
| | azaspiro[3.3]heptan-2-yl)isoxazol- | | 7.08 (m, 2H), 6.98 (d, J = 7.8 Hz, 2H), 6.87 |
| | 5-yl)-3-methylbutanoyl)-N-(4- | | (s, 1H), 5.85 (d, J = 4.9 Hz, 1H), 5.11 (d, J = |
| | fluorobenzyl)-4- | | 3.6 Hz, 1H), 4.91 (t, J = 8.8 Hz, 1H), 4.42- |
| | hydroxypyrrolidine-2-carboxamide | | 4.21 (m, 4H), 4.03 (s, 2H), 3.89-3.66 (d, J = |
| | | | 33.4 Hz, 4H), 3.60 (d, J = 9.8 Hz, 1H), |
| | | | 3.49-3.34 (m, 2H), 2.84 (dt, J = 33.1, 9.8 |
| | | | Hz, 4H), 20.48-2.42 (m, 3H), 2.35-2.14 |
| | | | (m, 1H), 2.10-1.95 (m, 2H), 1.93-1.80 |
| | | | (m, 2H), 0.99-0.88 (m, 3H), 0.86-0.76 |
| | | | (m, 3H). |
| 51 | (2S,4R)-N-((S)-sec-butyl)-1-(2-(3- | 696.35 | ¹H NMR (300 MHz, DMSO-d6) δ 14.17 (s, |
| | (6-(5-cyclobutyl-3-(2- | | 1H), 8.43 (s, 1H), 8.22-8.13 (m, 1H), 7.63 |
| | hydroxyphenyl)-5H-pyrrolo[3,2- | | (d, J = 8.4 Hz, 1H), 7.33 (t, J = 7.1 Hz, 1H), |
| | c]pyridazin-6-yl)-2- | | 7.00 (t, J = 7.3 Hz, 2H), 6.87 (s, 1H), 5.91- |
| | azaspiro[3.3]heptan-2-yl)isoxazol- | | 5.83 (m, 1H), 5.21-4.74 (m, 2H), 4.27 (dd, |
| | 5-yl)-3-methylbutanoyl)-4- | | J = 17.9, 10.1 Hz, 2H), 4.03 (s, 2H), 3.83 (d, |
| | hydroxypyrrolidine-2-carboxamide | | J = 5.4 Hz, 2H), 3.77-3.54 (m, 4H), 3.40 |
| | | | (d, J = 11.9 Hz, 1H), 2.99-2.65 (m, 4H), |
| | | | 2.44 (m, 4H), ., 2.05 (d, J = 10.1 Hz, 1H), |
| | | | 1.97 (s, 2H), 1.86 (q, J = 9.2 Hz, 2H), 1.42- |
| | | | 1.28 (m, 2H), 1.11-0.91 (m, 6H), 0.80 (dq, |
| | | | J = 9.1, 4.6, 4.0 Hz, 6H). |
| 52 | (2S,4R)-1-(2-(3-(6-(5-cyclobutyl-3- | 842.4 | ¹H NMR (400 MHz, DMSO-d6) δ 8.42 |
| | (2-hydroxyphenyl)-5H-pyrrolo[3,2- | | (s, 1H), 8.33 (d, J = 21.7 Hz, 1H), 8.20- |
| | c]pyridazin-6-yl)-2- | | 8.13 (m, 1H), 7.39-7.19 (m, 2H), |
| | azaspiro[3.3]heptan-2-yl)isoxazol- | | 7.13-6.95 (m, 4H), 6.87 (d, J = 6.9 Hz, |
| | 5-yl)-3-methylbutanoyl)-N-(4- | | 1H), 5.86 (d, J = 10.8 Hz, 1H), 4.96- |
| | ethynyl-2-(3- | | 4.87 (m, 1H), 4.35 (s, 1H), 4.26-4.12 |
| | methoxypropoxy)benzyl)-4- | | (m, 3H), 4.09-3.96 (m, 5H), 3.84 (s, |
| | hydroxypyrrolidine-2-carboxamide | | 1H), 3.81-3.67 (m, 3H), 3.64-3.54 |
| | | | (m, 3H), 3.53-3.45 (m, 3H), 3.48- |
| | | | 3.38 (m, 3H), 2.94-2.72 (m, 4H), 2.47 |
| | | | (s, 3H), 2.24 (s, 1H), 2.09-1.79 (m, |
| | | | 6H), 1.51 (s, 1H), 0.93 (dd, J = 12.6, 6.4 |
| | | | Hz, 3H), 0.81 (dd, J = 16.5, 6.6 Hz, 3H). |
| 53 | (2S,4R)-1-(2-(3-(6-(5-cyclobutyl-3- | 678.3 | ¹H NMR (400 MHz, Methanol-d4) δ |
| | (2-hydroxyphenyl)-5H-pyrrolo[3,2- | | 8.37 (s, 1H), 7.96 (d, J = 8.0 Hz, 1H), |
| | c]pyridazin-6-yl)-2- | | 7.36-7.27 (m, 1H), 7.05-6.96 (m, |
| | azaspiro[3.3]heptan-2-yl)isoxazol- | | 2H), 6.73 (s, 1H), 5.89-5.79 (m, 1H), |
| | 5-yl)-3-methylbutanoyl)-4- | | 4.99-4.90 (m, 1H), 4.53-4.38 (m, |
| | hydroxy-N-(prop-2-yn-1- | | 2H), 4.18-4.09 (m, 2H), 4.13-3.99 |
| | yl)pyrrolidine-2-carboxamide | | (m, 1H), 4.03-3.83 (m, 4H), 3.79- |
| | | | 3.52 (m, 3H), 3.02-2.78 (m, 4H), 2.69- |
| | | | 2.47 (m, 5H), 2.40-2.25 (m, 1H), |
| | | | 2.20-1.95 (m, 4H), 1.07-0.94 (m, |
| | | | 3H), 0.92-0.80 (m, 3H). |
| 54 | (2S,4R)-1-(2-(3-(6-(5-cyclobutyl-3- | 682.35 | ¹H NMR (400 MHz, DMSO-d6) δ 14.16 |
| | (2-hydroxyphenyl)-5H-pyrrolo[3,2- | | (s, 1H), 8.41 (s, 1H), 8.31-7.98 (m, |
| | c]pyridazin-6-yl)-2- | | 1H), 7.79-7.45 (m, 1H), 7.40-7.16 |
| | azaspiro[3.3]heptan-2-yl)isoxazol- | | (m, 1H), 7.04-6.90 (m, 2H), 6.86 (s, |
| | 5-yl)-3-methylbutanoyl)-4- | | 1H), 6.06-5.59 (m, 1H), 5.08 (d, J = |
| | hydroxy-N-isopropylpyrrolidine-2- | | 3.6 Hz, 1H), 5.01-4.77 (m, 1H), 4.55- |
| | carboxamide | | 4.12 (m, 2H), 4.03 (d, J = 3.0 Hz, 2H), |
| | | | 3.92-3.60 (m, 4H), 3.62-3.48 (m, |
| | | | 1H), 3.48-3.35 (m, 2H), 3.04-2.62 |
| | | | (m, 4H), 2.49-2.39 (m, 3H), 2.28- |
| | | | 2.15 (m, 1H), 2.13-1.98 (m, 1H), 2.00- |
| | | | 1.51 (m, 3H), 1.45-0.83 (m, 9H), |
| | | | 0.92-0.44 (m, 4H). |
| 55 | (2S,4R)-1-(2-(3-(6-(5-cyclobutyl-3- | 694.45 | ¹H NMR (300 MHz, DMSO-d6) δ 14.17 |
| | (2-hydroxyphenyl)-5H-pyrrolo[3,2- | | (d, J = 2.6 Hz, 1H), 8.42 (s, 1H), 8.22- |
| | c]pyridazin-6-yl)-2- | | 8.13 (m, 1H), 7.93 (t, J = 5.7 Hz, 1H), |
| | azaspiro[3.3]heptan-2-yl)isoxazol- | | 7.39-7.27 (m, 1H), 7.05-6.94 (m, |
| | 5-yl)-3-methylbutanoyl)-N- | | 2H), 6.87 (s, 1H), 5.91-5.82 (m, 1H), |
| | (cyclopropylmethyl)-4- | | 5.12-4.89 (m, 2H), 4.31 (dq, J = 15.6, |
| | hydroxypyrrolidine-2-carboxamide | | 7.6 Hz, 2H), 4.02 (d, J = 6.0 Hz, 2H), |
| | | | 3.87-3.67 (m, 4H), 3.61-3.40 (m, |
| | | | 2H), 3.07-2.74 (m, 6H), 2.44 (s, 2H), |
| | | | 2.20 (t, J = 8.2 Hz, 1H), 2.02 (dd, J = |
| | | | 19.4, 7.6 Hz, 2H), 1.86 (q, J = 9.0 Hz, |
| | | | 2H), 0.95 (dd, J = 6.6, 3.3 Hz, 3H), 0.89- |
| | | | 0.69 (m, 4H), 0.48-0.34 (m, 2H), |
| | | | 0.23-0.11 (m, 2H). |
| 56 | (2S,4R)-1-(2-(3-(6-(5-cyclobutyl-3- | 680.35 | ¹H NMR (300 MHz, DMSO-d6) δ 14.17 |
| | (2-hydroxyphenyl)-5H-pyrrolo[3,2- | | (s, 1H), 8.42 (s, 1H), 8.18 (d, 1H), 7.94 |
| | c]pyridazin-6-yl)-2- | | (d, J = 4.3 Hz, 1H), 7.32 (t, 1H), 7.05- |
| | azaspiro[3.3]heptan-2-yl)isoxazol- | | 6.94 (m, 2H), 6.87 (s, 1H), 5.86 (d, J = |
| | 5-yl)-3-methylbutanoyl)-N- | | 5.2 Hz, 1H), 5.13-4.82 (m, 2H), 4.40- |
| | cyclopropyl-4-hydroxypyrrolidine- | | 4.11 (m, 2H), 4.04 (d, J = 5.2 Hz, 2H), |
| | 2-carboxamide | | 3.84 (d, J = 4.6 Hz, 2H), 3.81-3.64 (m, |
| | | | 2H), 3.62-3.39 (m, 2H), 2.99-2.74 |
| | | | (m, 4H), 2.68-2.56 (m, 1H),2.47- |
| | | | 2.41 (m, 3H), 2.32-1.73 (m, 6H), 1.00- |
| | | | 0.82 (m, 3H), 0.85-0.69 (m, 3H), |
| | | | 0.60 (d, J = 6.7 Hz, 2H), 0.46-0.33 (m, |
| | | | 2H). |
| 57 | (2S,4R)-1-(2-(3-(6-(5-cyclobutyl-3- | 754.4 | ¹H NMR (400 MHz, DMSO-d6) δ 14.16 |
| | (2-hydroxyphenyl)-5H-pyrrolo[3,2- | | (s, 1H), 8.65-8.47 (m, 1H), 8.43 (d, J = |
| | c]pyridazin-6-yl)-2- | | 6.2 Hz, 2H), 8.22-8.10 (m, 1H), 7.46- |
| | azaspiro[3.3]heptan-2-yl)isoxazol- | | 7.37 (m, 2H), 7.36-7.18 (m, 3H), |
| | 5-yl)-3-methylbutanoyl)-N-(4- | | 7.05-6.91 (m, 2H), 6.89-6.77 (m, |
| | ethynylbenzyl)-4- | | 1H), 5.95-5.76 (m, 1H), 4.96-4.82 |
| | hydroxypyrrolidine-2-carboxamide | | (m, 1H), 4.48-4.20 (m, 4H), 4.16- |
| | | | 4.11 (m, 1H), 4.06-3.93 (m, 2H), 3.84 |
| | | | (s, 1H), 3.81-3.66 (m, 3H), 3.63-3.54 |
| | | | (m, 2H), 3.43 (s, 1H), 2.94-2.71 (m, |
| | | | 4H), 2.48-2.43 (m, 3H), 2.27-2.19 |
| | | | (m, 1H), 2.11-1.96 (m, 2H), 1.93- |
| | | | 1.73 (m, 2H), 1.05-0.85 (m, 3H), 0.85- |
| | | | 0.70 (m, 3H). |
| 58 | (2S,4R)-1-(2-(3-(6-(5-cyclobutyl-3- | 784.35 | ¹H NMR (400 MHz, DMSO-d6) δ 14.16 |
| | (2-hydroxyphenyl)-5H-pyrrolo[3,2- | | (s, 1H), 8.42 (s, 1H), 8.39-8.26 (m, |
| | c]pyridazin-6-yl)-2- | | 1H), 8.20-8.13 (m, 1H), 7.32 (td, J = |
| | azaspiro[3.3]heptan-2-yl)isoxazol- | | 7.7, 1.6 Hz, 1H), 7.30-7.17 (m, 1H), |
| | 5-yl)-3-methylbutanoyl)-N-(4- | | 7.15-6.95 (m, 4H), 6.86 (d, J = 5.6 Hz, |
| | ethynyl-2-methoxybenzyl)-4- | | 1H), 5.86 (d, J = 9.2 Hz, 1H), 5.12 (t, J = |
| | hydroxypyrrolidine-2-carboxamide | | 2.9 Hz, 1H), 4.99-4.87 (m, 1H), 4.42- |
| | | | 4.33 (m, 2H), 4.28-4.10 (m, 3H), |
| | | | 4.06-3.92 (m, 2H), 3.86-3.67 (m, |
| | | | 7H), 3.63-3.53 (m, 1H), 3.41 (d, J = |
| | | | 11.5 Hz, 1H), 2.95-2.73 (m, 4H), 2.46 |
| | | | (d, J = 9.6 Hz, 4H), 2.23 (d, J = 8.6 Hz, |
| | | | 1H), 2.03 (s, 2H), 1.95-1.80 (m, 2H), |
| | | | 0.92 (d, J = 6.6 Hz, 3H), 0.86-0.76 (m, |
| | | | 3H). |
| 59 | (2S,4R)-N-(2-chloro-4- | 788.35 | ¹H NMR (400 MHz, DMSO-d6) δ 14.16 |
| | ethynylbenzyl)-1-(2-(3-(6-(5- | | (s, 1H), 8.55 (dt, J = 29.0, 5.9 Hz, 1H), |
| | cyclobutyl-3-(2-hydroxyphenyl)- | | 8.42 (s, 1H), 8.20-8.13 (m, 1H), 7.61- |
| | 5H-pyrrolo[3,2-c]pyridazin-6-yl)-2- | | 7.36 (m, 3H), 7.36-7.25 (m, 1H), 7.03- |
| | azaspiro[3.3]heptan-2-yl)isoxazol- | | 6.95 (m, 2H), 6.89-6.83 (m, 1H), |
| | 5-yl)-3-methylbutanoyl)-4- | | 5.90-5.81 (m, 1H), 5.16-5.10 (m, |
| | hydroxypyrrolidine-2-carboxamide | | 1H), 5.04-4.87 (m, 1H), 4.50-4.20 |
| | | | (m, 5H), 4.07-3.90 (m, 2H), 3.86- |
| | | | 3.67 (m, 4H), 3.64-3.55 (m, 1H), 3.43 |
| | | | (d, J = 11.3 Hz, 1H), 2.84 (dp, J = 34.0, |
| | | | 12.2, 11.4 Hz, 4H), 2.48-2.44 (m, 3H), |
| | | | 2.27-2.22 (m, 2H), 2.08-2.01 (m, |
| | | | 2H), 1.96-1.80 (m, 2H), 0.94 (dd, J = |
| | | | 22.7, 6.6 Hz, 3H), 0.86-0.60 (m, 3H). |
| 60 | (2S,4R)-1-(2-(3-(6-(5-cyclobutyl-3- | 768.45 | ¹H NMR (300 MHz, DMSO-d6) δ 14.17 |
| | (2-hydroxyphenyl)-5H-pyrrolo[3,2- | | (d, J = 2.7 Hz, 1H), 8.40 (d, J = 13.5 Hz, |
| | c]pyridazin-6-yl)-2- | | 2H), 8.28-8.13 (m, 1H), 7.41 (dd, J = |
| | azaspiro[3.3]heptan-2-yl)isoxazol- | | 8.1, 5.0 Hz, 2H), 7.29 (t, J = 8.9 Hz, |
| | 5-yl)-3-methylbutanoyl)-N-((S)-1- | | 3H), 6.99 (dd, J = 8.1, 6.6 Hz, 2H), 6.87 |
| | (4-ethynylphenyl)ethyl)-4- | | (s, 1H), 5.87 (d, J = 4.0 Hz, 1H), 5.09 |
| | hydroxypyrrolidine-2-carboxamide | | (d, J = 3.6 Hz, 1H),5.03-4.75 (m, 2H), |
| | | | 4.32 (dd, J = 19.5, 11.7 Hz, 2H), 4.13 |
| | | | (d, J = 1.5 Hz, 1H), 4.07-3.78 (m, 5H) |
| | | | 3.78-3.65 (m, 2H), 3.62-3.49 (m, |
| | | | 2H), 3.49-3.3 (m, 2H), 2.97-2.68 (m, |
| | | | 4H), 2.31-2.15 (m, 2H), 2.05 (d, J = |
| | | | 14.3 Hz, 2H), 1.93-1.79 (m, 1H), 1.75 |
| | | | (d, J = 4.3 Hz, 1H), 1.33 (t, J = 6.3 Hz, |
| | | | 3H), 0.95 (t, J = 5.4 Hz, 3H), 0.80 (dd, J = |
| | | | 9.7, 6.6 Hz, 3H). |
| 63 | (2S,4R)-1-((R)-2-(3-(6-(5- | 762.35 | ¹H NMR (300 MHz, DMSO-d6) δ 14.17 |
| | cyclobutyl-3-(2-hydroxyphenyl)- | | (s, 1H), 8.43 (s, 1H), 8.36 (d, J = 7.7 |
| | 5H-pyrrolo[3,2-c]pyridazin-6-yl)-2- | | Hz, 1H), 8.18 (d, J = 7.5 Hz, 1H), 7.31 |
| | azaspiro[3.3]heptan-2-yl)isoxazol- | | (dd, J = 8.5, 5.5 Hz, 3H), 7.13 (t, J = 8.9 |
| | 5-yl)-3-methylbutanoyl)-N-((S)-1- | | Hz, 2H), 6.99 (d, J = 7.9 Hz, 2H), 6.88 |
| | (4-fluorophenyl)ethyl)-4- | | (s, 1H), , 5.87 (s, 1H),5.10 (d, J = 3.6 |
| | hydroxypyrrolidine-2-carboxamide | | Hz, 1H), 4.96-4.86 (m, 2H), 4.32 (dd, |
| | | | J = 17.5, 9.7 Hz, 2H), 4.04 (s, 2H), 3.84 |
| | | | (s, 2H), 3.74-3.64 (m, 2H), 3.59 (d, J = |
| | | | 9.8 Hz, 1H), 3.51 (d, J = 9.8 Hz, 1H), |
| | | | 3.17 (d, J = 5.2 Hz, 1H), 2.92-2.79 (m, |
| | | | 4H)2.30-2.06 (m, 2H), 2.05 (d, J = |
| | | | 10.7 Hz, 3H), 1.96 (dd, J = 55.5, 10.0 |
| | | | Hz, 3H). 1.38 (dd, J = 23.0, 7.3 Hz, 3H), |
| | | | 0.95 (d, J = 6.5 Hz, 3H), 0.79 (d, J = 6.8 |
| | | | Hz, 3H). |
| 118 | (2S,4R)-N-cyclobutyl-1-(2-(3-(6- | 694.35 | ¹H NMR (400 MHz, DMSO-d6) δ 14.16 |
| | (5-cyclobutyl-3-(2-hydroxyphenyl)- | | (s, 1H), 8.48-8.40 (m, 1H), 8.20-7.93 |
| | 5H-pyrrolo[3,2-c]pyridazin-6-yl)-2- | | (m, 2H), 7.37-7.28 (m, 1H), 7.03- |
| | azaspiro[3.3]heptan-2-yl)isoxazol- | | 6.95 (m, 2H), 6.87 (s, 1H), 5.90-5.82 |
| | 5-yl)-3-methylbutanoyl)-4- | | (m, 1H), 5.09 (d, J = 3.7 Hz, 1H), 5.01- |
| | hydroxypyrrolidine-2-carboxamide | | 4.87 (m, 1H), 4.52-3.98 (m, 5H), 3.90- |
| | | | 3.62 (m, 4H), 3.62-3.33 (m, 2H), |
| | | | 2.93-2.74 (m, 4H), 2.49-2.42 (m, |
| | | | 3H), 2.35-1.72 (m, 10H), 1.67-1.56 |
| | | | (m, 2H), 0.97-0.90 (m, 3H), 0.84- |
| | | | 0.75 (m, 3H). |
| 119 | (2S,4R)-1-(2-(3-(6-(5-cyclobutyl-3- | 708.35 | ¹H NMR (300 MHz, DMSO-d6) δ 14.17 |
| | (2-hydroxyphenyl)-5H-pyrrolo[3,2- | | (s, 1H), 8.42 (s, 1H), 8.20-8.12 (m, |
| | c]pyridazin-6-yl)-2- | | 1H), 7.73 (dd, J = 47.3, 7.3 Hz, 1H), |
| | azaspiro[3.3]heptan-2-yl)isoxazol- | | 7.39-7.28 (m, 1H), 7.06-6.95 (m, |
| | 5-yl)-3-methylbutanoyl)-N- | | 2H), 6.87 (s, 1H), 5.89-5.73 (m, 1H), |
| | cyclopentyl-4-hydroxypyrrolidine- | | 5.09 (d, J = 3.6 Hz, 1H), 5.01-4.85 (m, |
| | 2-carboxamide | | 1H), 4.41-4.20 (m, 2H), 4.07-3.90 |
| | | | (m, 3H), 3.84 (d, J = 4.8 Hz, 2H), 3.79- |
| | | | 3.65 (m, 2H), 3.61-3.41 (m, 2H), 2.96- |
| | | | 2.72 (m, 4H), 2.46 (d, J = 9.3 Hz, 3H), |
| | | | 2.20 (d, J = 8.4 Hz, 1H), 2.07 (t, J = |
| | | | 10.5 Hz, 1H), 2.01-1.91 (m, 1H), 1.89- |
| | | | 1.69 (m, 4H), 1.64 (s, 2H), 1.57-1.12 |
| | | | (m, 5H), 1.01-0.68 (m, 6H). |
| 120 | (2S,4R)-N-(bicyclo[1.1.1]pentan- | 706.35 | ¹H NMR (400 MHz, DMSO-d6) δ 14.18 |
| | 1-yl)-1-(2-(3-(6-(5-cyclobutyl-3-(2- | | (d, J = 3.6 Hz, 1H), 8.48-8.27 (m, 2H), |
| | hydroxyphenyl)-5H-pyrrolo[3,2- | | 8.21-8.14 (m, 1H), 7.36-7.28 (m, 1H), |
| | c]pyridazin-6-yl)-2- | | 6.99 (t, J = 7.5 Hz, 2H), 6.87 (s, 1H), |
| | azaspiro[3.3]heptan-2-yl)isoxazol- | | 5.90-5.79 (m, 1H), 5.10 (d, J = 3.7 Hz, |
| | 5-yl)-3-methylbutanoyl)-4- | | 1H), 5.02-4.83 (m, 1H), 4.47-4.24 (m, |
| | hydroxypyrrolidine-2-carboxamide | | 1H), 4.23-4.08 (m, 1H), 4.03 (d, J = |
| | | | 5.2 Hz, 2H), 3.83 (d, J = 5.3 Hz, 2H), |
| | | | 3.78-3.49 (m, 2H), 3.49-3.18 (m, 2H), |
| | | | 2.89 (q, J = 10.2 Hz, 4H), 2.83-2.63 |
| | | | (m, 4H), 2.58-2.51 (m, 1H), 2.47 (m, |
| | | | 1H), 2.45-2.12 (m, 2H), 2.12-1.95 (m, |
| | | | 6H), 1.94-1.75 (m, 2H), 0.94 (dd, J = |
| | | | 8.6, 5.3 Hz, 3H), 0.87-0.76 (m, 3H). |
| 121 | (2S,4R)-N-((S)-1-(2-chloro-4- | 802.25 | ¹H NMR (400 MHz, DMSO-d6) δ 14.75- |
| | ethynylphenyl)ethyl)-1-(2-(3-(6-(5- | | 13.92 (m, 1H), 9.13-8.34 (m, 2H), |
| | cyclobutyl-3-(2-hydroxyphenyl)- | | 8.27-8.05 (m, 1H), 7.65-7.11 (m, |
| | 5H-pyrrolo[3,2-c]pyridazin-6-yl)-2- | | 4H), 6.99 (t, J = 7.5 Hz, 2H), 6.86 (d, J = |
| | azaspiro[3.3]heptan-2-yl)isoxazol- | | 2.8 Hz, 1H), 6.11-5.57 (m, 1H), 5.33- |
| | 5-yl)-3-methylbutanoyl)-4- | | 4.97 (m, 2H), 4.93-4.79 (m, 1H), |
| | hydroxypyrrolidine-2-carboxamide | | 4.73-4.21 (m, 3H), 4.03 (s, 2H), 3.83 |
| | | | (d, J = 2.3 Hz, 2H), 3.78-3.52 (m, 3H), |
| | | | 3.13-2.60 (m, 4H), 2.46 (d, J = 10.3 |
| | | | Hz, 2H), 2.36-2.11 (m, 1H), 2.12- |
| | | | 1.98 (m, 2H), 1.87 (p, J = 9.6 Hz, 1H), |
| | | | 1.79-1.64 (m, 1H), 1.52-1.37 (m, |
| | | | 1H), 1.35-1.26 (m, 3H), 1.24 (d, J = |
| | | | 7.1 Hz, 2H), 1.07-0.86 (m, 3H), 0.83- |
| | | | 0.65 (m, 3H). |

### Preparation of (2S,4R)-1-((R)-2-(4-(2-((S)-3-(5-cyclopropyl-3-(2-hydroxyphenyl)-5H-pyrrolo[3,2-c]pyridazin-6-yl)pyrrolidin-1-yl)pyrimidin-5-yl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide (Compound 75) and (2S,4R)-1-((S)-2-(4-(2-((S)-3-(5-cyclopropyl-3-(2-hydroxyphenyl)-5H-pyrrolo[3,2-c]pyridazin-6-yl)pyrrolidin-1-yl)pyrimidin-5-yl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide (Compound 74)

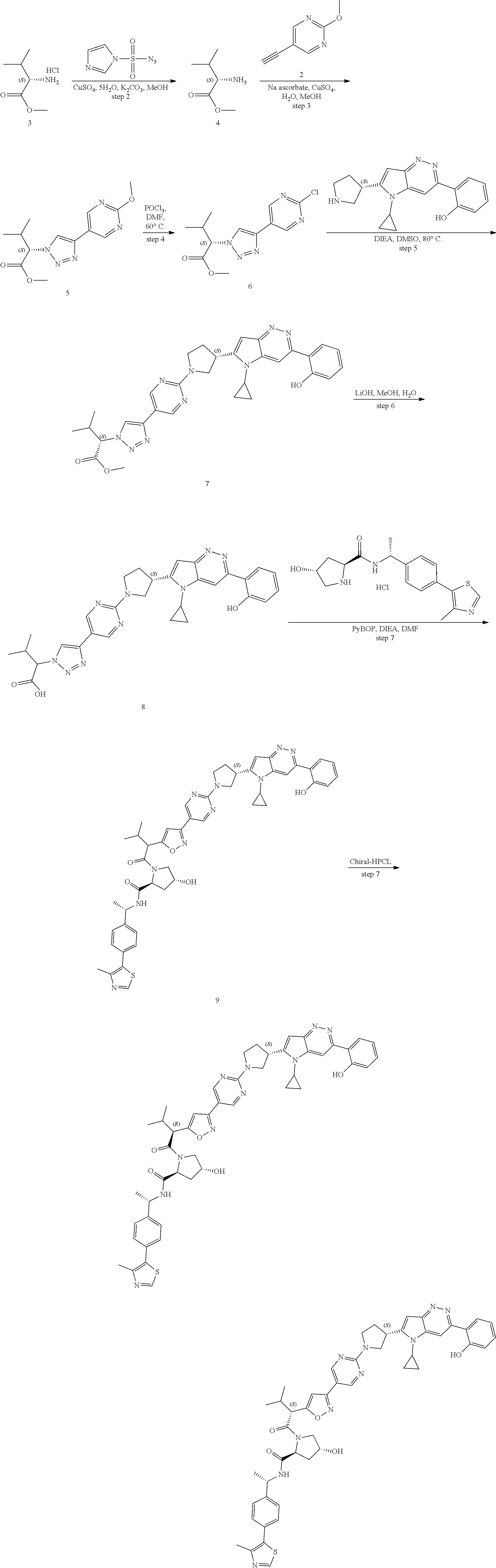

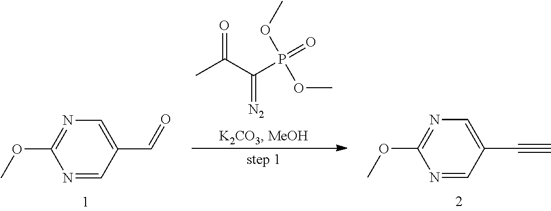

### Step 1: Preparation of 5-ethynyl-2-methoxypyrimidine (Intermediate 2)

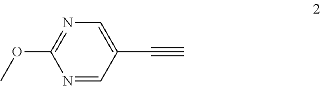

To a stirred solution of 2-methoxypyrimidine-5-carbaldehyde (5 g, 36.199 mmol, 1 equiv) and K₂CO₃ (10.01 g, 72.398 mmol, 2.0 equiv) in MeOH (50 mL) was added dimethyl (1-diazo-2-oxopropyl)phosphonate (8.35 g, 43.439 mmol, 1.2 equiv) dropwise at 0° C. under nitrogen atmosphere. The resulting mixture was stirred for 2 h at room temperature. Desired product could be detected by LCMS. The resulting mixture was diluted with water (200 mL). The resulting mixture was extracted with EtOAc (3×500 mL). The combined organic layers were washed with brine (300 mL), dried over anhydrous Na₂SO₄. After filtration, the filtrate was concentrated under reduced pressure. This resulted in Intermediate 2 (4.8 g, 98.85%) as a light-yellow solid. LCMS (ESI) m/z: [M+H]⁺=135.

### Step 2: Preparation of methyl (2S)-2-azido-3-methylbutanoate (Intermediate 4)

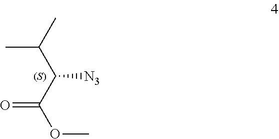

To a stirred mixture of methyl (2S)-2-amino-3-methylbutanoate hydrochloride (5 g, 29.828 mmol, 1 equiv), CuSO₄·5H₂O (0.74 g, 2.983 mmol, 0.1 equiv) and K₂CO₃ (12.37 g, 89.484 mmol, 3 equiv) in MeOH (10 mL) was added imidazole-1-sulfonyl azide (9.38 g, 44.742 mmol, 1.5 equiv) in portions at 0° C. under nitrogen atmosphere. The resulting mixture was stirred for overnight at room temperature under nitrogen atmosphere. Desired product could be detected by LCMS. The resulting mixture was diluted with water (300 mL) extracted with EtOAc (3×300 mL). The combined organic layers were washed with brine (3×100 mL), dried over anhydrous Na₂SO₄. After filtration, the filtrate was concentrated under reduced pressure to afford intermediate 4 (6.62 g, crude) as a colorless liquid. The crude product was used in the next step directly without further purification. LCMS (ESI) m/z: [M+H]⁺=158.

### Step 3: Preparation of methyl (2S)-2-[4-(2-methoxypyrimidin-5-yl)-1,2,3-triazol-1-yl]-3-methylbutanoate (Intermediate 5)

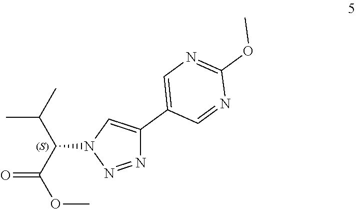

A mixture of intermediate 4 (5 g, 31.812 mmol, 1 equiv), intermediate 2 (4.27 g, 31.812 mmol, 1 equiv), sodium (5R)-5-[(1S)-1,2-dihydroxyethyl]-3,4-dihydroxy-2,5-dihydrofuran-2-one (3.17 g, 15.906 mmol, 0.5 equiv) and CuSO₄·5H₂O (0.79 g, 3.181 mmol, 0.1 equiv) in MeOH (10 mL) and H₂O (5 mL) was stirred for overnight at room temperature. Desired product could be detected by LCMS. The resulting mixture was diluted with water (300 mL). The resulting mixture was extracted with EtOAc (3×300 mL). The combined organic layers were washed with brine (3×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 30 min; detector, UV 254 nm to afford intermediate 5 (6.4 g, 69.06%) as a yellow oil. LCMS (ESI) m/z [M+H]⁺=292.

### Step 4: Preparation of methyl (2S)-2-[4-(2-chloropyrimidin-5-yl)-1,2,3-triazol-1-yl]-3-methylbutanoate (Intermediate 6)

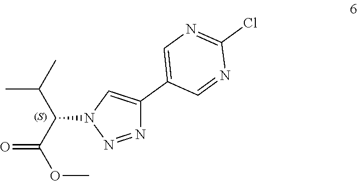

To a stirred solution of intermediate 5 (7.7 g, 26.432 mmol, 1 equiv) in DMF (70 mL) was added POCl₃ (20.26 g, 132.160 mmol, 5 equiv) dropwise at 0° C. under nitrogen atmosphere. The resulting mixture was stirred for overnight at 60° C. under nitrogen atmosphere. Desired product could be detected by LCMS. The reaction was quenched with Water/Ice at 0° C. The resulting mixture was extracted with EtOAc (3×300 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (3:1) to afford intermediate 6 (1.92 g, 24.56%) as a yellow oil. LCMS (ESI) m/z: [M+H]⁺=296.

### Step 5: Preparation of methyl (2S)-2-(4-{2-[(3S)-3-[5-cyclopropyl-3-(2-hydroxyphenyl)pyrrolo[3,2-c]pyridazin-6-yl]pyrrolidin-1-yl]pyrimidin-5-yl}-1,2,3-triazol-1-yl)-3-methylbutanoate (Intermediate 7)

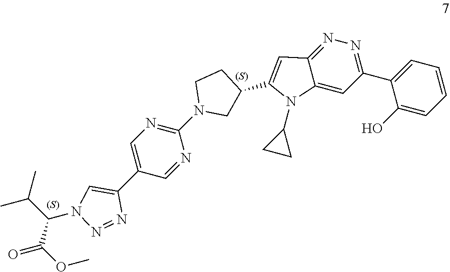

A mixture of intermediate 6 (300 mg, 1.014 mmol, 1 equiv), 2-{5-cyclopropyl-6-[(3S)-pyrrolidin-3-yl]pyrrolo[3,2-c]pyridazin-3-yl}phenol (650.04 mg, 2.028 mmol, 2 equiv) and DIEA (393.34 mg, 3.042 mmol, 3 equiv) in DMF (3 mL) was stirred for 2 h at 80° C. under nitrogen atmosphere. Desired product could be detected by LCMS. The residue was purified by reversed-phase flash chromatography with the following conditions: column, C18 silica gel; mobile phase, MeCN in Water (0.1% FA), 0% to 100% gradient in 30 min; detector, UV 254 nm to afford intermediate 7 (330 mg, 56.12%) as a yellow solid. LCMS (ESI) m/z: [M+H]⁺=580.

### Step 6: Preparation of 2-(4-(2-((S)-3-(5-cyclopropyl-3-(2-hydroxyphenyl)-5H-pyrrolo[3,2-c]pyridazin-6-yl)pyrrolidin-1-yl)pyrimidin-5-yl)-1H-1,2,3-triazol-1-yl)-3-methylbutanoic acid (Intermediate 8)

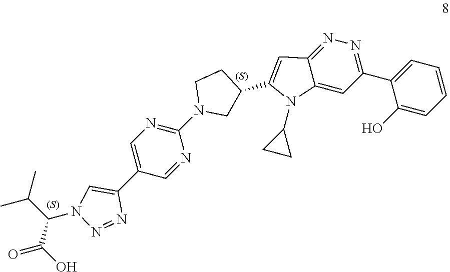

A mixture of intermediate 7 (320 mg, 0.552 mmol, 1 equiv) and LiOH·H₂O (115.82 mg, 2.760 mmol, 5 equiv) in MeOH (2 mL), THF (2 mL) and H₂O (1 mL) was stirred for 2 h at room temperature. Desired product could be detected by LCMS. The mixture was acidified to pH 6 with HCl (aq.). The resulting mixture was extracted with EtOAc (3×100 mL). The combined organic layers were washed with brine (3×10 mL), dried over anhydrous Na₂SO₄. After filtration, the filtrate was concentrated under reduced pressure to afford intermediate 8 (340 mg, crude) as a yellow solid. LCMS (ESI) m/z: [M+H]⁺=566.

### Step 7: Preparation of (2S,4R)-1-[(2S)-2-(4-{2-[(3S)-3-[5-cyclopropyl-3-(2-hydroxyphenyl)pyrrolo[3,2-c]pyridazin-6-yl]pyrrolidin-1-yl]pyrimidin-5-yl}-1,2,3-triazol-1-yl)-3-methylbutanoyl]-4-hydroxy-N-[(1S)-1-[4-(4-methyl-1,3-thiazol-5-yl)phenyl]ethyl]pyrrolidine-2-carboxamide (Intermediate 9)

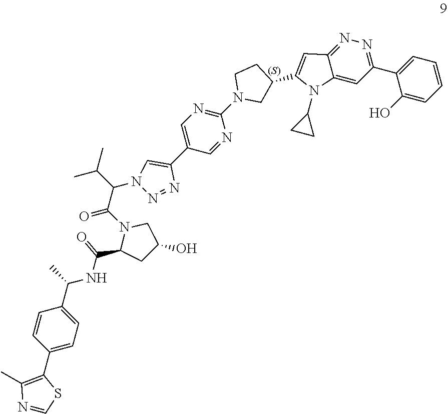

A mixture of intermediate 8 (300 mg, 0.530 mmol, 1 equiv), (2S,4R)-4-hydroxy-N-[(1R)-1-[4-(4-methyl-1,3-thiazol-5-yl)phenyl]ethyl]pyrrolidine-2-carboxamide (263.67 mg, 0.795 mmol, 1.5 equiv), PyBOP (414.01 mg, 0.795 mmol, 1.5 equiv) and DIEA (274.20 mg, 2.120 mmol, 4 equiv) in DMF (3 mL) was stirred for 2 h at room temperature. Desired product could be detected by LCMS. The crude product was purified by Prep-HPLC with the following conditions (Column: Xselect CSH C18 OBD Column 30*150 mm 5 μm; Mobile Phase A: water (0.1% TFA), Mobile Phase B: ACN; Flow rate: 60 mL/min; Gradient: 30% B to 48% B in 8 min, 48% B; Wave Length: 254/220 nm; RT1(min): 8.77) to afford intermediate 9 (350 mg, 75.07%) as a yellow solid. LCMS (ESI) m/z: [M+H]⁺=879.

### Step 8: Preparation of (2S,4R)-1-[(2R)-2-(4-{2-[(3S)-3-[5-cyclopropyl-3-(2-hydroxyphenyl)pyrrolo[3,2-c]pyridazin-6-yl]pyrrolidin-1-yl]pyrimidin-5-yl}-1,2,3-triazol-1-yl)-3-methylbutanoyl]-4-hydroxy-N-[(1S)-1-[4-(4-methyl-1,3-thiazol-5-yl)phenyl]ethyl]pyrrolidine-2-carboxamide (Compound 75) and (2S,4R)-1-[(2S)-2-(4-{2-[(3S)-3-[5-cyclopropyl-3-(2-hydroxyphenyl)pyrrolo[3,2-c]pyridazin-6-yl]pyrrolidin-1-yl]pyrimidin-5-yl}-1,2,3-triazol-1-yl)-3-methylbutanoyl]-4-hydroxy-N-[(1S)-1-[4-(4-methyl-1,3-thiazol-5-yl)phenyl]ethyl]pyrrolidine-2-carboxamide (Compound 74)

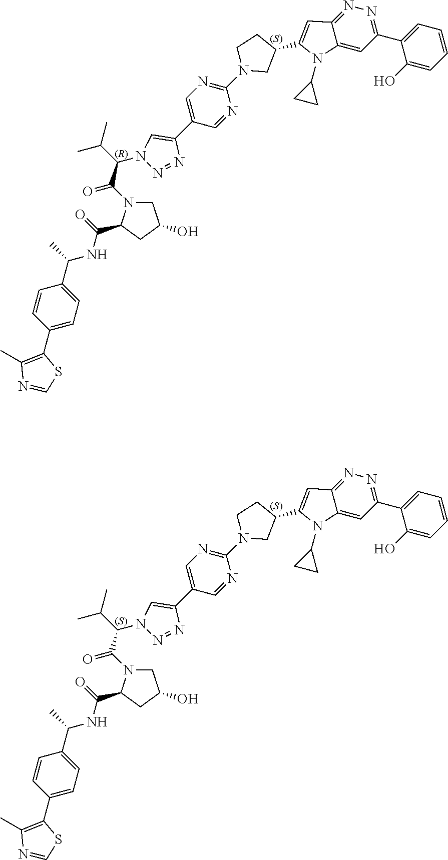

Intermediate 9 was purified by Chiral-HPLC with the following conditions (Column: CHIRALPAK ID, 2*25 cm, 5 μm; Mobile Phase A: MeOH:DCM=1:1 (0.1% 2M NH3-MEOH), Mobile Phase B: MeOH--HPLC; Flow rate: 20 mL/min; Gradient: 50% B to 50% B in 50 min; Wave Length: 268/222 nm; RT1(min): 10; RT2(min): 29.5; Sample Solvent: MeOH:DCM=1:1--HPLC; Injection Volume: 2 mL). This resulted in:

75 (102.0 mg, 33.39%) as a light-yellow solid. ¹H NMR (400 MHz, DMSO-d6) δ 14.28-14.23 (m, 1H), 9.13-8.82 (m, 3H), 8.67-8.61 (m, 1H), 8.37 (s, 1H), 8.23 (dd, J=56.8, 8.0 Hz, 2H), 7.52-7.20 (m, 5H), 7.04-6.96 (m, 2H), 6.94-6.88 (m, 1H), 5.48-4.72 (m, 3H), 4.45 (t, J=7.9 Hz, 1H), 4.33-4.22 (m, 2H), 4.14 (q, J=7.7 Hz, 1H), 3.93-3.83 (m, 1H), 3.80-3.66 (m, 2H), 3.64-3.56 (m, 1H), 3.51-3.45 (m, 1H), 2.64-2.60 (m, 3H), 2.39 (s, 3H), 2.37-2.29 (m, 1H), 2.16-2.05 (m, 1H), 2.01-1.73 (m, 1H), 1.51-1.29 (m, 5H), 1.26-1.18 (m, 2H), 0.98 (dd, J=53.2, 6.6 Hz, 3H), 0.71 (dd, J=18.3, 6.6 Hz, 3H). LCMS (ESI) m/z: [M+H]⁺=879.40.

74 (132.5 mg, 43.59%) as an off-white solid. ¹H NMR (400 MHz, DMSO-d6) δ 14.25 (s, 1H), 9.00-8.86 (m, 3H), 8.71-8.64 (m, 1H), 8.53 (d, J=7.6 Hz, 1H), 8.37 (s, 1H), 8.16 (d, J=7.9 Hz, 1H), 7.48-7.29 (m, 5H), 7.04-6.95 (m, 2H), 6.90 (s, 1H), 5.39-5.08 (m, 2H), 4.99-4.80 (m, 1H), 4.40 (t, J=8.1 Hz, 1H), 4.36-4.24 (m, 2H), 4.12 (p, J=7.6 Hz, 1H), 3.94-3.84 (m, 1H), 3.83-3.65 (m, 3H), 3.53-3.45 (m, 1H), 2.69-2.57 (m, 2H), 2.46 (s, 3H), 2.40-2.27 (m, 2H), 2.12-2.05 (m, 1H), 1.85-1.74 (m, 1H), 1.58-1.36 (m, 3H), 1.36-1.31 (m, 2H), 1.27-1.16 (m, 2H), 1.09 (d, J=6.5 Hz, 3H), 0.74 (d, J=6.5 Hz, 3H). LCMS (ESI) m/z: [M+H]⁺=879.40.

Compounds in Table 14 were prepared using a procedure similar to the one used above for the preparation of 75 and 74 using the appropriate amines.

TABLE 14

| | | LCMS (ESI) | |
|---|---|---|---|
| No. | Name | m/z | ¹H NMR |
| 72 | (2S,4R)-1-((R)-2-(4-(2-((R)-3-(5- | 879.35 | ¹H NMR (400 MHz, DMSO-d6) δ 14.26 (s, |
| | cyclopropyl-3-(2-hydroxyphenyl)- | | 1H), 8.95 (s, 1H), 8.86 (s, 2H), 8.64 (d, J = |
| | 5H-pyrrolo[3,2-c]pyridazin-6- | | 2.6 Hz, 1H), 8.37 (s, 1H), 8.30 (d, J = 7.9 |
| | yl)pyrrolidin-1-yl)pyrimidin-5-yl)- | | Hz, 1H), 8.16 (d, J = 7.9 Hz, 1H), 7.57- |
| | 1H-1,2,3-triazol-1-yl)-3- | | 7.12 (m, 5H), 7.00 (t, J = 7.8 Hz, 2H), 6.91 |
| | methylbutanoyl)-4-hydroxy-N-((S)- | | (d, J = 8.7 Hz, 1H), 5.43 (d, J = 9.5 Hz, |
| | 1-(4-(4-methylthiazol-5- | | 1H), 5.19 (d, J = 3.6 Hz, 1H), 4.85 (t, J = |
| | yl)phenyl)ethyl)pyrrolidine-2- | | 7.3 Hz, 1H), 4.45 (t, J = 7.9 Hz, 1H), 4.26 |
| | carboxamide | | (dd, J = 10.9, 7.2 Hz, 2H), 4.17-4.09 (m, |
| | | | 1H), 3.87 (s, 1H), 3.80-3.68 (m, 3H), |
| | | | 3.80-3.68 (m, 1H), 3.59 (dd, J = 10.8, 4.3 |
| | | | Hz, 1H), 2.47-2.4 (m, 1H), 2.4-2.3 (m, |
| | | | 1H), 2.39 (s, 2H), 2.09 (d, J = 11.9 Hz, |
| | | | 1H), 1.79 (dq, J = 13.1, 7.6, 6.2 Hz, 1H), |
| | | | 1.49 (d, J = 7.0 Hz, 1H), 1.33 (dd, J = 9.9, |
| | | | 5.8 Hz, 5H), 1.22 (d, J = 9.7 Hz, 2H), 0.98 |
| | | | (dd, J = 53.1, 6.6 Hz, 3H), 0.71 (dd, J = |
| | | | 18.3, 6.6 Hz, 3H). |
| 71 | (2S,4R)-1-((S)-2-(4-(2-((R)-3-(5- | 879.35 | ¹H NMR (400 MHz, DMSO-d6) δ 8.99 (s, |
| | cyclopropyl-3-(2-hydroxyphenyl)- | | 1H), 8.89 (s, 2H), 8.69 (s, 1H), 8.53 (d, J = |
| | 5H-pyrrolo[3,2-c]pyridazin-6- | | 7.6 Hz, 1H), 8.37 (s, 1H), 8.16 (d, J = 8.0 |
| | yl)pyrrolidin-1-yl)pyrimidin-5-yl)- | | Hz, 1H), 7.45-7.34 (dd 5H), 7.00 (t, J = |
| | 1H-1,2,3-triazol-1-yl)-3- | | 8.0 Hz, 2H), 6.91 (s, 1H), 5.36 (d, J = 10.2 |
| | methylbutanoyl)-4-hydroxy-N-((S)- | | Hz, 1H), 5.19 (d, J = 3.7 Hz, 1H), 4.93 (t, J = |
| | 1-(4-(4-methylthiazol-5- | | 7.2 Hz, 1H), 4.40-4.3 (m, 3H), 4.28 (t, J = |
| | yl)phenyl)ethyl)pyrrolidine-2- | | 9.1 Hz, 1H), 4.17-4.08 (m, 1H), 3.88 |
| | carboxamide | | (d, J = 8.1 Hz, 1H), 3.82-3.67 (m, 4H), |
| | | | 3.54 (m, 1h) 2.63 (d, J = 6.5 Hz, 1H), |
| | | | 2.47 (d, J = 6.7 Hz, 3H), 2.31-2.10 (m, |
| | | | 1H), 2.12-2.02 (m, 1H), 1.85-1.74 (m, |
| | | | 1H), 1.45-1.34 (m, 5H), 1.22 (d, J = 11.6 |
| | | | Hz, 3H), 1.13-1.05 (m, 3H), 0.74 (d, J = |
| | | | 6.6 Hz, 3H). |

### Example 2. Degradation of BRM and BRG1 by Compounds of the Invention

This example demonstrates the ability of the compounds of the disclosure to degrade a HiBit-BRM or HiBit-BRG1 fusion protein in a cell-based degradation assay.

Procedure: A stable HeLa cell line expressing HiBiT-BRM was generated. On day 0, 5000 cells were seeded in 40 μL of media into each well of 384-well cell culture plates. On day 1, cells were treated with 120 nL DMSO or 120 nL of 3-fold serially DMSO-diluted compounds (10 points in duplicate with 30 μM as final top dose). Subsequently plates were incubated for 24 h in a standard tissue culture incubator and equilibrated at room temperature for 15 minutes. Nano-GB HiBiT Lytic Detection System (Promega N3050) reagent was freshly prepared and 20 ul was added to each well. Upon addition of this LgBit-containing reagent, the HiBiT and LgBiT proteins associate to form the luminescent NanoBiT luciferase. The plates were shaken for 10 minutes at room temperature and the bioluminescence read using an EnVision plate reader (PerkinElmer).

For measurement of BRG1 degradation, a stable HeLa cell line expressing HiBit-BRG1 and LgBit was generated. The same protocol as above was then followed.

The degradation % was calculated using the following formula: % degradation=100%−100%×(Lumₛₐₘₚₗₑ−LuM_{LC})/(LuM_{HC}−LuM_{LC}). DMSO treated cells are employed as High Control (HC) and 2 μM of a known BRM/BRG1 degrader standard treated cells are employed as Low Control (LC). The data was fit to a four parameter, non-linear curve fit to calculate IC₅₀ (PM) values as shown in Table 15.

Results: As shown in Table 15 below, the compounds of the invention degraded BRM and/or BRG1.

TABLE 15

| Com- | BRM HiBit | BRM HiBit | BRMG1 HiBit | BRG1 HiBit |
|---|---|---|---|---|
| pound | Degradation | Degradation | Degradation | Degradation |
| No. | IC50 (nM) | Maximum (%) | IC50 (nM) | Maximum (%) |
| 1 | +++ | A | ++ | A |
| 2 | ++ | A | NC | C |
| 3 | + | B | NC | C |
| 4 | +++ | A | ++ | B |
| 5 | ++++ | A | ++ | A |
| 6 | +++ | A | ++ | A |
| 7 | ++ | A | NC | C |
| 8 | ++++ | A | +++ | A |
| 9 | +++ | A | NC | C |
| 10 | +++ | A | + | B |
| 11 | +++ | A | NC | C |
| 12 | ++++ | A | +++ | A |
| 13 | ++++ | A | ++ | B |
| 14 | +++ | A | +++ | B |
| 15 | ++ | A | NC | C |
| 16 | NC | C | NC | C |
| 17 | ++++ | A | +++ | A |
| 18 | ++++ | A | ++++ | A |
| 19 | +++ | B | NC | C |
| 20 | ++++ | A | ++++ | A |
| 21 | ++++ | A | ++++ | B |
| 22 | ++ | B | NC | C |
| 23 | ++++ | A | +++ | B |
| 24 | +++ | B | NC | C |
| 25 | ++++ | A | NC | C |
| 26 | NC | C | NC | C |
| 27 | ++++ | A | +++ | A |
| 28 | ++++ | A | ++++ | A |
| 29 | +++ | A | NC | C |
| 30 | +++ | A | ++ | A |
| 31 | +++ | B | NC | C |
| 32 | NC | C | NC | C |
| 33 | NC | C | NC | C |
| 34 | ++++ | A | NC | C |
| 35 | ++ | A | NC | C |
| 36 | ++++ | A | ++++ | A |
| 37 | +++ | A | NC | C |
| 38 | NT | | NT | |
| 39 | NT | | NT | |
| 40 | NC | C | NC | C |
| 41 | +++ | A | NC | C |
| 42 | +++ | B | NC | C |
| 43 | ++++ | A | NC | C |
| 44 | ++++ | B | NC | C |
| 45 | ++++ | A | ++++ | A |
| 46 | NC | C | NC | C |
| 47 | ++++ | A | NC | C |
| 48 | +++ | A | NC | C |
| 49 | +++ | A | NC | C |
| 50 | +++ | A | NO | C |
| 51 | +++ | A | NC | C |
| 52 | ++++ | A | NC | C |
| 53 | +++ | A | NC | C |
| 54 | +++ | A | NC | C |
| 55 | +++ | A | NC | C |
| 56 | +++ | A | NC | C |
| 57 | ++++ | A | NC | C |
| 58 | ++++ | A | NC | C |
| 59 | +++ | A | NC | C |
| 60 | ++++ | A | NC | C |
| 61 | +++ | A | NC | C |
| 62 | ++++ | A | +++ | B |
| 63 | +++ | A | NC | C |
| 64 | ++++ | A | NC | C |
| 65 | ++++ | A | NC | C |
| 66 | ++++ | A | +++ | B |
| 67 | ++++ | A | +++ | B |
| 68 | ++++ | A | NC | C |
| 69 | ++++ | A | NC | C |
| 70 | ++++ | A | NC | C |
| 71 | ++++ | A | ++++ | A |
| 72 | ++++ | A | NC | C |
| 73 | +++ | B | +++ | B |
| 74 | ++++ | A | ++++ | B |
| 75 | +++ | A | NC | C |
| 76 | ++++ | A | NC | C |
| 77 | ++++ | A | NC | C |
| 78 | ++++ | A | NC | C |
| 79 | +++ | A | NC | C |
| 80 | +++ | A | +++ | A |
| 81 | ++++ | A | ++++ | A |
| 82 | ++++ | A | +++ | B |
| 83 | +++ | A | NC | C |
| 84 | ++++ | A | NC | C |
| 85 | ++++ | A | +++ | A |
| 86 | ++++ | A | NC | C |
| 87 | ++++ | A | ++++ | A |
| 88 | ++++ | A | +++ | A |
| 89 | +++ | A | +++ | B |
| 90 | ++++ | A | NC | C |
| 91 | +++ | A | NC | C |
| 92 | ++++ | A | NC | C |
| 93 | ++++ | A | NC | C |
| 94 | ++++ | A | NO | C |
| 95 | ++++ | A | +++ | B |
| 96 | ++++ | A | +++ | B |
| 97 | ++++ | A | NC | C |
| 98 | +++ | A | +++ | A |
| 99 | ++++ | A | +++ | B |
| 100 | +++ | A | NC | C |
| 101 | ++++ | A | +++ | B |
| 102 | ++++ | A | NC | C |
| 103 | ++++ | A | ++++ | B |
| 104 | ++++ | A | ++++ | A |
| 105 | ++++ | A | NC | C |
| 106 | +++ | B | NC | C |
| 107 | +++ | A | NC | C |
| 108 | +++ | B | NC | C |
| 109 | ++++ | A | NC | C |
| 110 | ++++ | A | NC | C |
| 111 | ++++ | A | NC | C |
| 112 | +++ | A | +++ | B |
| 113 | ++++ | A | +++ | B |
| 114 | ++++ | A | +++ | A |
| 115 | ++++ | A | +++ | B |
| 116 | +++ | A | NC | C |
| 117 | ++++ | A | NC | C |
| 118 | +++ | A | NC | C |
| 119 | +++ | A | NC | C |
| 120 | +++ | A | NC | C |
| 121 | +++ | A | NC | C |

“+” indicates inhibitory effect of ≥1000 nM; “++” indicates inhibitory effect of ≥100 nM; “+++” indicates inhibitory effect of ≥10 nM; “++++” indicates inhibitory effect of <10 nM; “NT” indicates not tested; “NC” indicates not calculated; “A” indicates maximum degradation ≥75%; “B” indicates maximum degradation ≥50%; and “C” indicates maximum degradation <50%.

### Other Embodiments

Embodiment 1. A compound, or a pharmaceutically acceptable salt thereof, of Formula I:

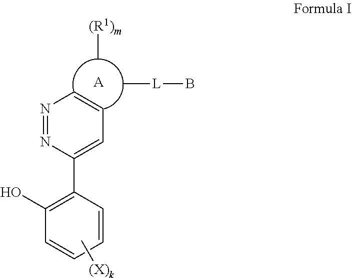

wherein
ring system A is a 5 to 9-membered heterocyclyl or heteroaryl containing at least one N;
m is 0, 1, 2, or 3;
k is 0, 1, or 2;
each R¹ is, independently, halo, optionally substituted C₁-C₆ alkyl, optionally substituted C₁-C₆ heteroalkyl, optionally substituted C₂-C₉ heterocyclyl, optionally substituted C₃-C₈ cycloalkyl or optionally substituted CH₂—C₃-C₈ cycloalkyl;
each X is, independently, halo;
L is a linker; and
B is a degradation moiety.

Embodiment 2. The compound of Embodiment 1, or a pharmaceutically acceptable salt thereof, wherein the compound has the structure of Formula I-A:

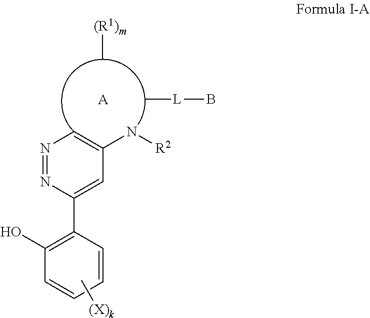

wherein R² is H, optionally substituted C₁-C₆ alkyl, optionally substituted C₃-C₈ cycloalkyl, optionally substituted C₂-C₉ heterocyclyl, or a bond to -L-B.

Embodiment 3. The compound of Embodiment 2, or a pharmaceutically acceptable salt thereof, of Formula I-B:

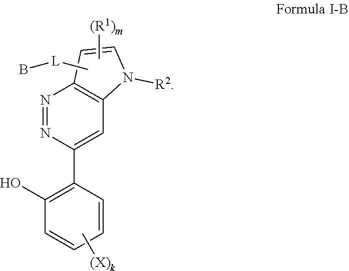

Embodiment 4. The compound of Embodiment 3, or a pharmaceutically acceptable salt thereof, wherein the compound has the structure of Formula I-C:

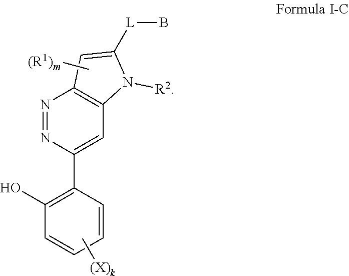

Embodiment 5. The compound of any one of Embodiments 1 to 4, or a pharmaceutically acceptable salt thereof, wherein R² is H.

Embodiment 6. The compound of any one of Embodiments 1 to 4, or a pharmaceutically acceptable salt thereof, wherein R² is optionally substituted C₁-C₆ alkyl.

Embodiment 7. The compound of any one of Embodiments 1 to 4, or a pharmaceutically acceptable salt thereof, wherein R² is optionally substituted C₃-C₈ cycloalkyl.

Embodiment 8. The compound of any one of Embodiments 1 to 4, or a pharmaceutically acceptable salt thereof, wherein R² is optionally substituted C₂-C₉ heterocyclyl.

Embodiment 9. The compound of any one of Embodiments 1 to 4, or a pharmaceutically acceptable salt thereof, R² is H, CH₃,

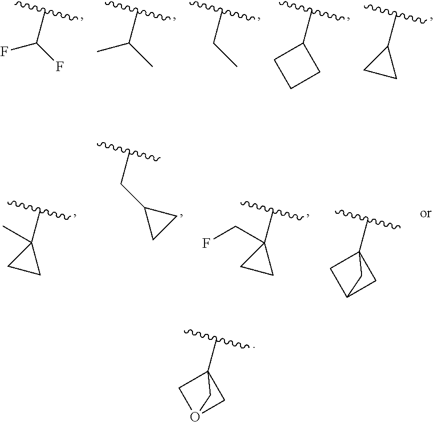

Embodiment 10. The compound of Embodiment 9, or a pharmaceutically acceptable salt thereof, wherein R² is H, CH₃,

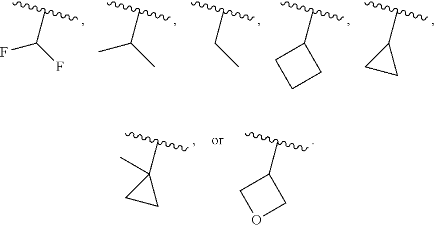

Embodiment 11. The compound of Embodiment 3, or a pharmaceutically acceptable salt thereof, wherein the compound has the structure of Formula I-D:

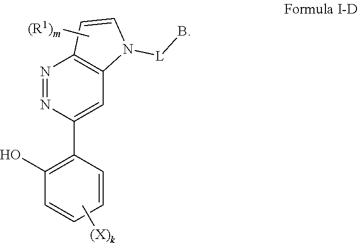

Embodiment 12. The compound of any one of Embodiments 1 to 11, or a pharmaceutically acceptable salt thereof, wherein m is 1.

Embodiment 13. The compound of any one of Embodiments 1 to 11, or a pharmaceutically acceptable salt thereof, wherein m is 2

Embodiment 14. The compound of any one of Embodiments 1 to 13, or a pharmaceutically acceptable salt thereof, wherein R¹ is optionally substituted C₁-C₆ alkyl.

Embodiment 15. The compound of Embodiment 14, or a pharmaceutically acceptable salt thereof, wherein R¹ is methyl or difluoromethyl.

Embodiment 16. The compound of any one of Embodiments 1 to 11, or a pharmaceutically acceptable salt thereof, wherein m is 0.

Embodiment 17. The compound of any one of Embodiments 1 to 16, or a pharmaceutically acceptable salt thereof, wherein k is 0.

Embodiment 18. The compound of any one of Embodiments 1 to 16, or a pharmaceutically acceptable salt thereof, wherein k is 1.

Embodiment 19. The compound of any one of Embodiments 1 to 16, or a pharmaceutically acceptable salt thereof, wherein k is 2.

Embodiment 20. The compound of any one of Embodiments 1-16 and 18-19, or a pharmaceutically acceptable salt thereof, wherein X is Cl or F.

Embodiment 21. The compound of any one of Embodiments 1 to 20, or a pharmaceutically acceptable salt thereof, wherein the compound has the structure of formula I-E, formula I-F, formula I-G, formula I-H or formula I-I:

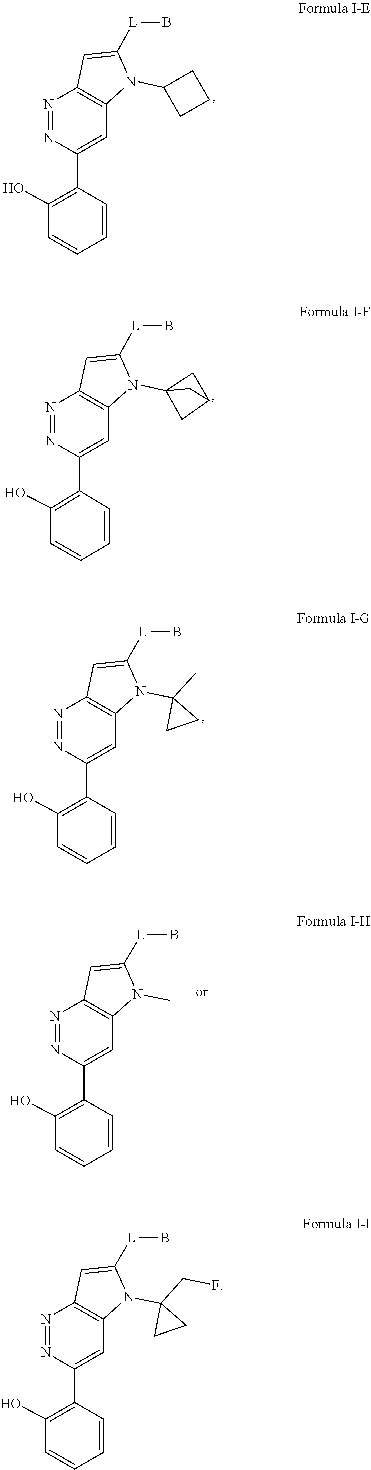

Embodiment 22. The compound of any one of Embodiments 1 to 20, or a pharmaceutically acceptable salt thereof, wherein the compound has the structure of formula -J, formula I-K, formula I-L, formula I-M or formula I-N:

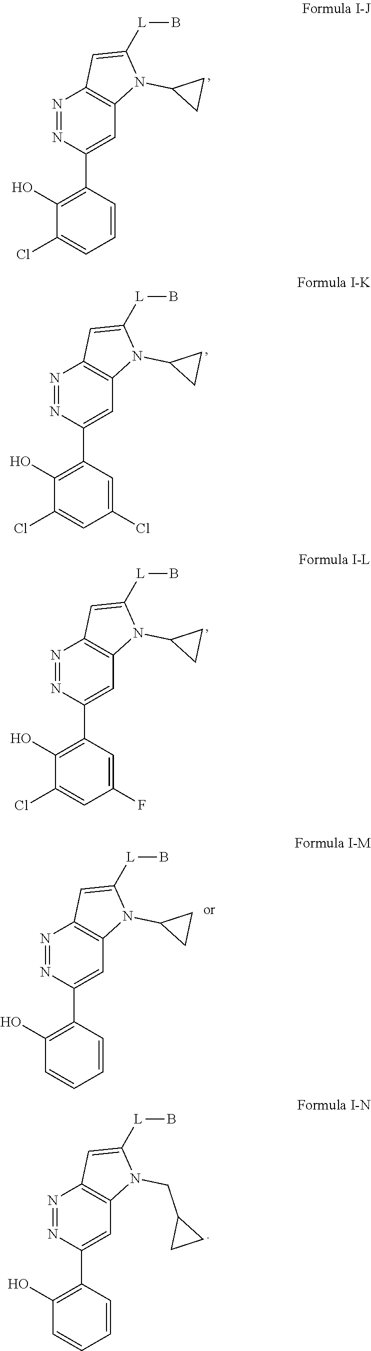

Embodiment 23. The compound of any one of Embodiments 1 to 20, or a pharmaceutically acceptable salt thereof, wherein the compound has the structure of formula 1-0, formula I-P, formula I-Q, formula I-R or formula I-S:

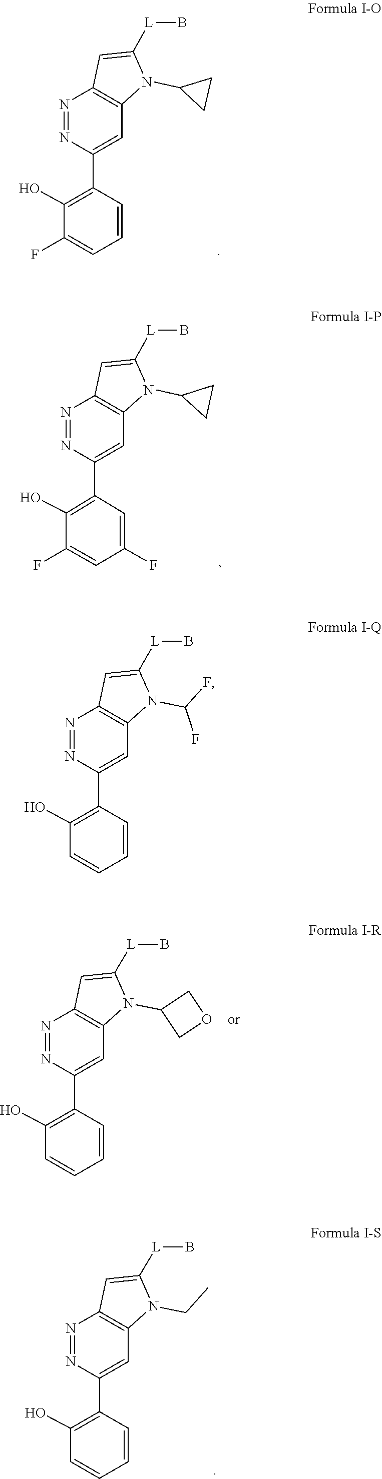

Embodiment 24. The compound of any one of Embodiments 1 to 20, or a pharmaceutically acceptable salt thereof, wherein the compound has the structure of formula I-T, formula I-U, formula I-V or formula I-W:

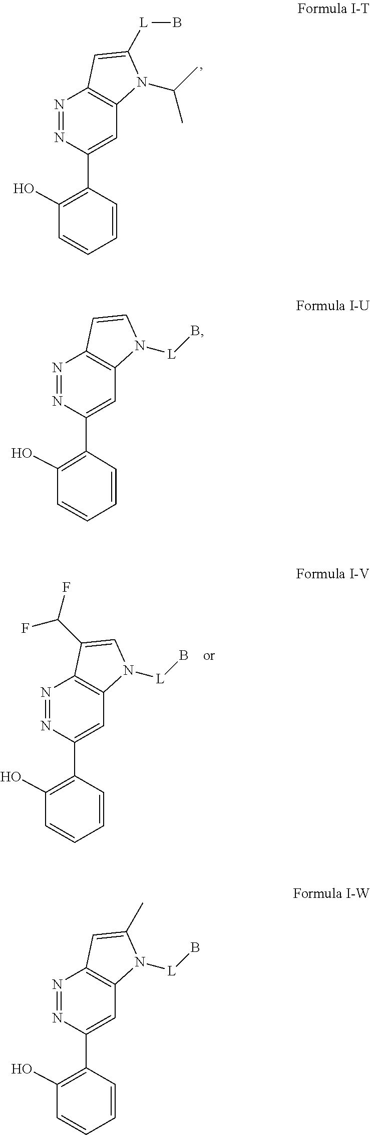

Embodiment 25. The compound of any one of Embodiments 1 to 24, or a pharmaceutically acceptable salt thereof, wherein the degradation moiety, B, has the structure of Formula A-1:

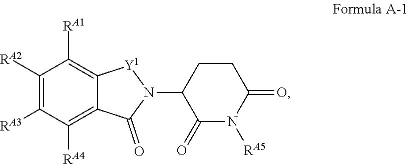

wherein
Y¹ is

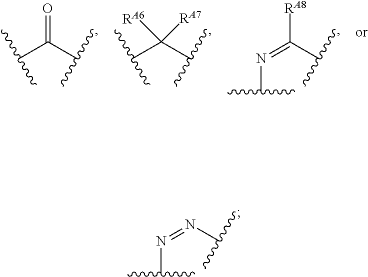

R^{A5} is H, optionally substituted C₁-C₆ alkyl, or optionally substituted C₁-C₆ heteroalkyl;
R^{A6} is H or optionally substituted C₁-C₆ alkyl; and R^{A7} is H or optionally substituted C₁-C₆ alkyl; or R^{A6} and R^{A7} together with the carbon atom to which each is bound combine to form optionally substituted C₃-C₆ carbocyclyl or optionally substituted C₂-C₅ heterocyclyl; or R^{A6} and R^{A7}, together with the carbon atom to which each is bound, combine to form optionally substituted C₃-C₆ carbocyclyl or optionally substituted C₂-C₅ heterocyclyl;
R^{A8} is H, optionally substituted C₁-C₆ alkyl, or optionally substituted C₁-C₆ heteroalkyl;
each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

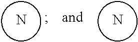

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A²,
where one of R^{A1}, R^{A2}, R^{A3}, and R^{A4} is A², or

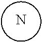

is substituted with A²; and
A² is a bond between the degradation moiety and the linker.

Embodiment 26. The compound of Embodiment 25, or a pharmaceutically acceptable salt thereof, wherein R^{A5} is H or methyl.

Embodiment 27. The compound of any one of Embodiments 25 to 26, or a pharmaceutically acceptable salt thereof, wherein R^{A1} is A² and each of R^{A2}, R^{A3}, and R^{A4} is H.

Embodiment 28. The compound of any one of Embodiments 25 to 26, or a pharmaceutically acceptable salt thereof, wherein R^{A2} is A² and each of R^{A1}, R^{A3}, and R^{A4} is H.

Embodiment 29. The compound of any one of Embodiments 25 to 26, or a pharmaceutically acceptable salt thereof, wherein R^{A3} is A² and each of R^{A1}, R^{A2} and R^{A4} is H.

Embodiment 30. The compound of any one of Embodiments 25 to 26, or a pharmaceutically acceptable salt thereof, wherein R^{A4} is A² and each of R^{A1}, R^{A2} and R^{A3} is H.

Embodiment 31. The compound of any one of Embodiments 25 to 30, or a pharmaceutically acceptable salt thereof, wherein Y¹ is

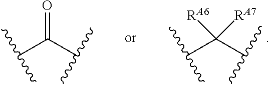

Embodiment 32. The compound of Embodiment 31, or a pharmaceutically acceptable salt thereof, wherein R^{A6} is H, and R^{A7} is H.

Embodiment 33. The compound of any one of Embodiments 25 to 30, or a pharmaceutically acceptable salt thereof, wherein Y¹ is

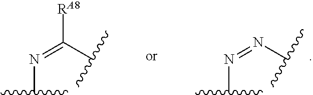

Embodiment 34. The compound of Embodiment 33, or a pharmaceutically acceptable salt thereof, wherein R^{A8} is H or methyl.

Embodiment 35. The compound of any one of Embodiments 25 to 28, or a pharmaceutically acceptable salt thereof, wherein the degradation moiety has the structure of Formula A2 or Formula A4:

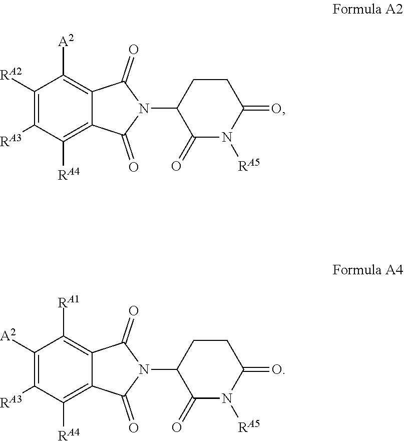

Embodiment 36. The compound of Embodiment 35, or a pharmaceutically acceptable salt thereof, wherein the degradation moiety is

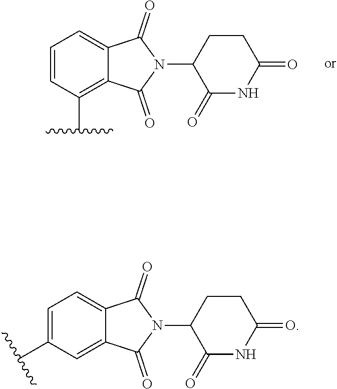 [SMILES: *c1cccc2c1C(=O)N(C1CCC(=O)NC1=O)C2=O ; "or" ; *c1ccc2c(c1)C(=O)N(C1CCC(=O)NC1=O)C2=O]

Embodiment 37. The compound of any one of Embodiments 25 to 30, or a pharmaceutically acceptable salt thereof, wherein the degradation moiety has the structure of Formula A5, Formula A6, Formula A8, or Formula A10:

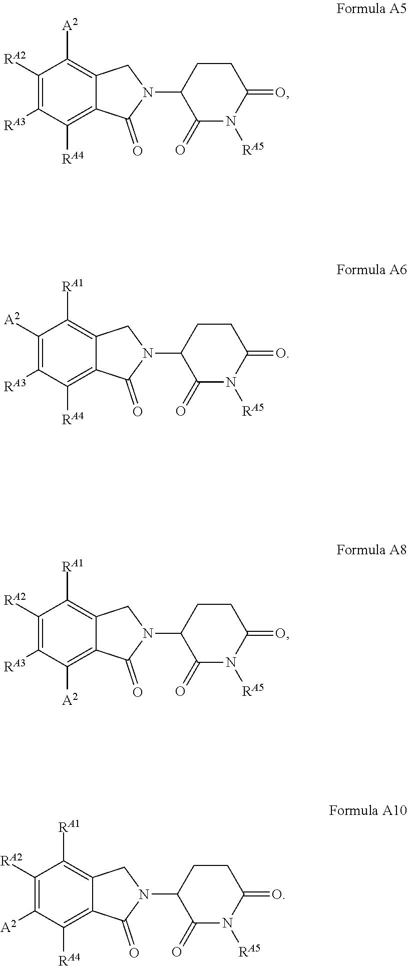

Embodiment 38. The compound of Embodiment 25, or a pharmaceutically acceptable salt thereof, wherein the degradation moiety has the structure of

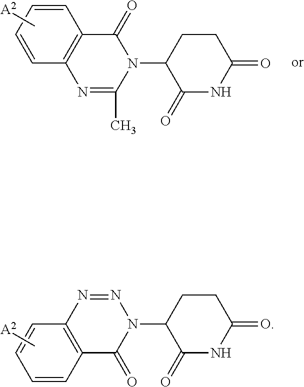

Embodiment 39. The compound of any one of Embodiments 1 to 24, or a pharmaceutically acceptable salt thereof, wherein the degradation moiety has the structure of Formula C:

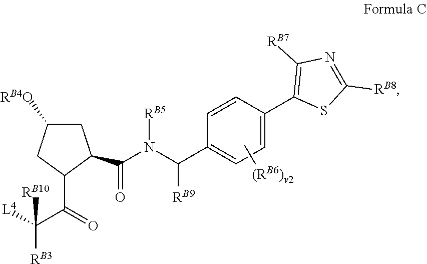

wherein
L⁴ is —N(R^{B1})(R^{B2}),

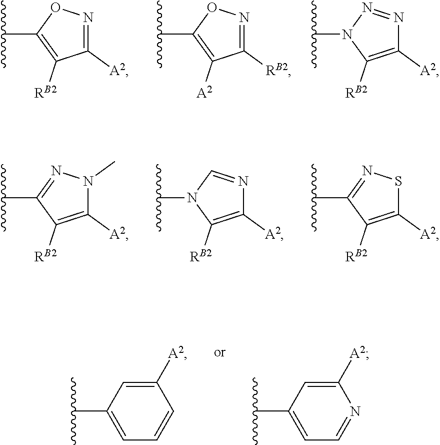

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₂-C₆ alkynyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl;
R^{B10} is H or F; and
A² is a bond between the degradation moiety and the linker;
wherein one and only one of R^{B1}, R^{B3}, and R^{B6} is A²,
or a pharmaceutically acceptable salt thereof.

Embodiment 40. The compound of any one of Embodiments 1 to 24, or a pharmaceutically acceptable salt thereof, wherein the degradation moiety has the structure of Formula C:

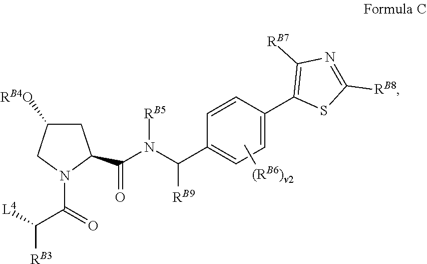

wherein
L⁴ is —N(R^{B1})(R^{B2}),

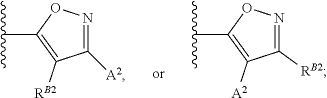

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl; and
A² is a bond between the degradation moiety and the linker;
wherein one and only one of R^{B1}, R^{B3}, and R^{B6} is A²,
or a pharmaceutically acceptable salt thereof.

Embodiment 41. The compound of Embodiments 39-40, or a pharmaceutically acceptable salt thereof, wherein the degradation moiety has the structure of Formula C3 or Formula C1:

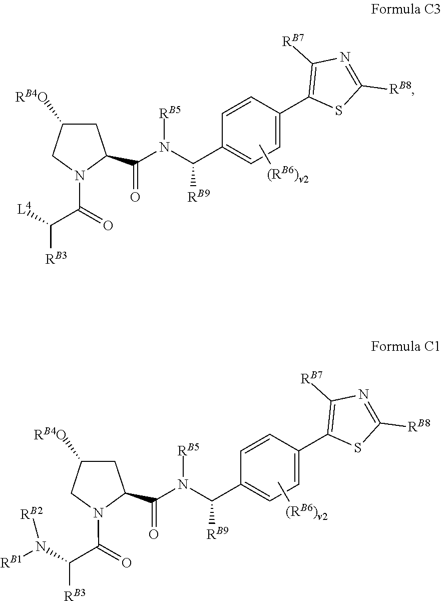

Embodiment 42. The compound of Embodiment 39, or a pharmaceutically acceptable salt thereof, wherein the degradation moiety has the structure of Formula C4:

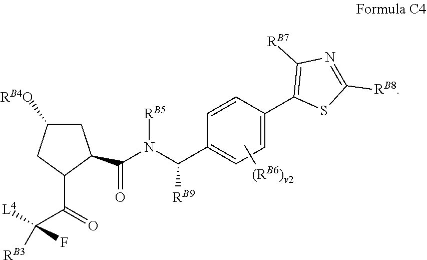

Embodiment 43. The compound of any one of Embodiments 39-40, or a pharmaceutically acceptable salt thereof, wherein the degradation moiety is

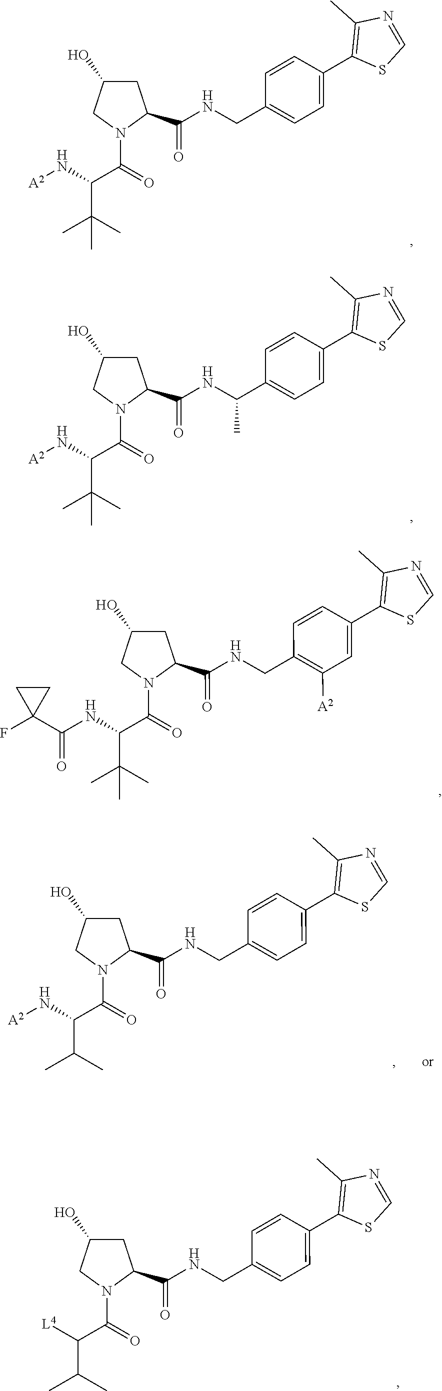

Embodiment 44. The compound of Embodiment 39, or a pharmaceutically acceptable salt thereof, wherein the degradation moiety is

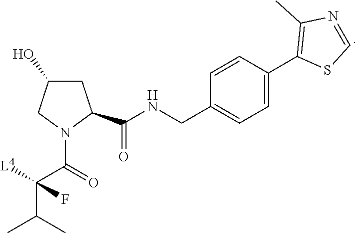

Embodiment 45. The compound of any one of Embodiments 39-40, or a pharmaceutically acceptable salt thereof, wherein the degradation moiety has the structure of Formula C2:

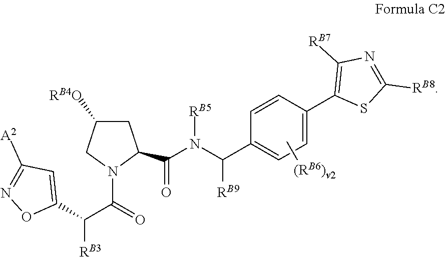

Embodiment 46. The compound Embodiment 39, or a pharmaceutically acceptable salt thereof, wherein the degradation moiety has the structure of Formula Ca2, Formula Cb2, Formula Cc2 Formula Cd2, Formula Ce2 or Formula Cf2:

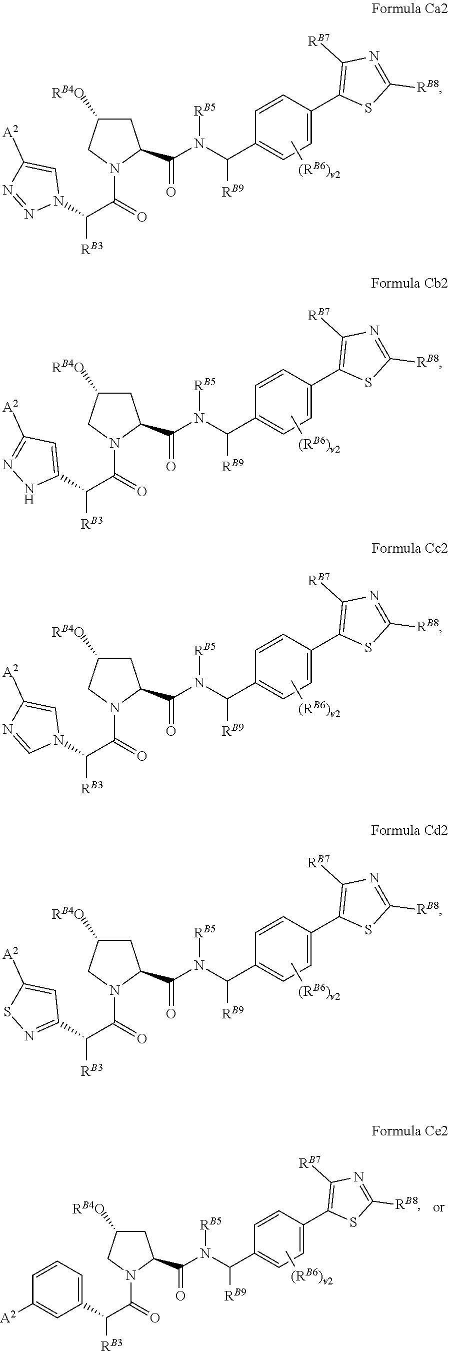

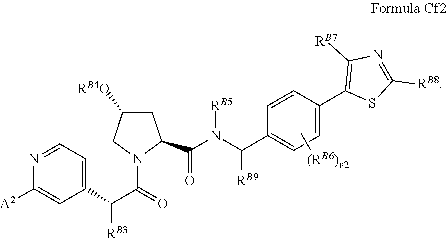

Embodiment 47. The compound of any one of Embodiments 39-42, and 45-46, or a pharmaceutically acceptable salt thereof, wherein R^{B9} is optionally substituted C₁-C₆ alkyl.

Embodiment 48. The compound of Embodiment 47, or a pharmaceutically acceptable salt thereof, wherein R^{B9} is methyl.

Embodiment 49. The compound of any one of Embodiments 39-42, and 45-48, or a pharmaceutically acceptable salt thereof, wherein R^{B9} is bonded to (S)-stereogenic center.

Embodiment 50. The compound of any one of Embodiments 39-42, and 45-49, or a pharmaceutically acceptable salt thereof, wherein v2 is 0.

Embodiment 51. The compound of any one of Embodiments 39-42, and 45-50, or a pharmaceutically acceptable salt thereof, wherein R^{B4} is H.

Embodiment 52. The compound of any one of Embodiments 39-42, and 45-51, or a pharmaceutically acceptable salt thereof, wherein R^{B5} is H.

Embodiment 53. The compound of any one of Embodiments 39-42, and 45-52, or a pharmaceutically acceptable salt thereof, wherein R^{B7} is optionally substituted C₁-C₆ alkyl.

Embodiment 54. The compound of Embodiment 53, or a pharmaceutically acceptable salt thereof, wherein R^{B7} is methyl.

Embodiment 55. The compound of any one of Embodiments 39-42, and 45-54, or a pharmaceutically acceptable salt thereof, wherein R^{B3} is optionally substituted C₁-C₆ alkyl.

Embodiment 56. The compound of Embodiment 55, or a pharmaceutically acceptable salt thereof, wherein R^{B3} is isopropyl or fluoro-2-methylpropane.

Embodiment 57. The compound of any one of Embodiments 39-42 and 45-54, or a pharmaceutically acceptable salt thereof, wherein R^{B3} is optionally substituted C₃-C₁₀ carbocyclyl.

Embodiment 58. The compound of Embodiment 57, or a pharmaceutically acceptable salt thereof, wherein R^{B3} is cyclopropane.

Embodiment 59. The compound of any one of Embodiments 39-42, and 45-58, or a pharmaceutically acceptable salt thereof, wherein R^{B8} is H.

Embodiment 60. The compound of any one of Embodiments 39-42, and 45-59, or a pharmaceutically acceptable salt thereof, wherein R^{B2} is H.

Embodiment 61. The compound of any one of Embodiments 39-40, or a pharmaceutically acceptable salt thereof, wherein the degradation moiety is

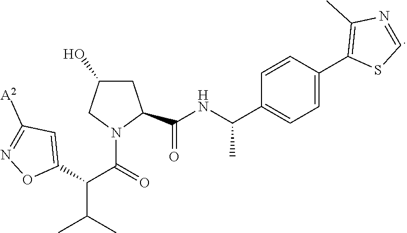

Embodiment 62. The compound of any one of Embodiments 39, or a pharmaceutically acceptable salt thereof, wherein the degradation moiety is

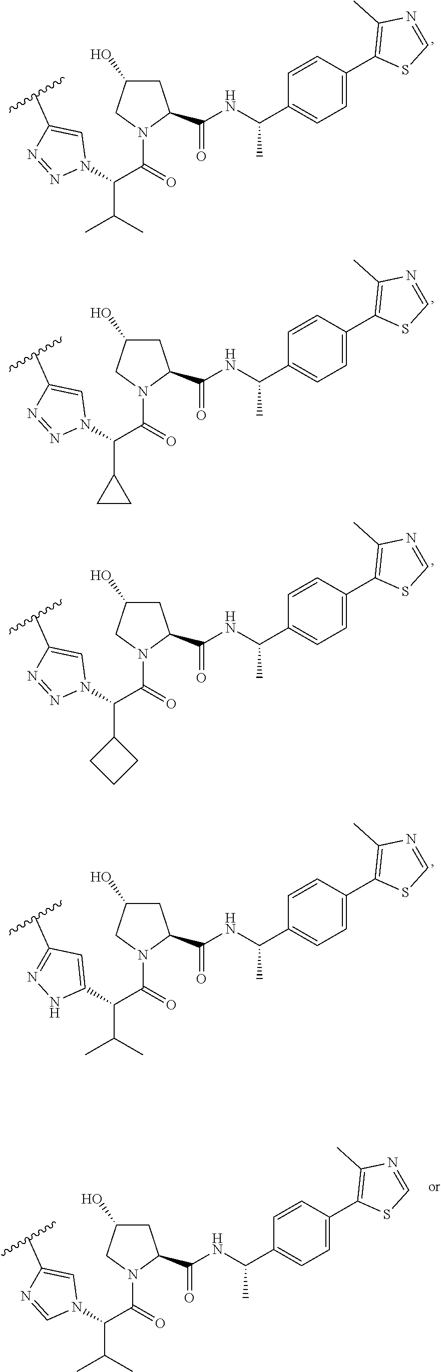 [SMILES: *c1cn([C@H](C(=O)N2C[C@H](O)C[C@H]2C(=O)N[C@@H](C)c2ccc(-c3scnc3C)cc2)C(C)C)nn1 ; *c1cn([C@H](C(=O)N2C[C@H](O)C[C@H]2C(=O)N[C@@H](C)c2ccc(-c3scnc3C)cc2)C2CC2)nn1 ; *c1cn([C@H](C(=O)N2C[C@H](O)C[C@H]2C(=O)N[C@@H](C)c2ccc(-c3scnc3C)cc2)C2CCC2)nn1 ; *c1cc([C@H](C(=O)N2C[C@H](O)C[C@H]2C(=O)N[C@@H](C)c2ccc(-c3scnc3C)cc2)C(C)C)[nH]n1 ; *c1cn([C@H](C(=O)N2C[C@H](O)C[C@H]2C(=O)N[C@@H](C)c2ccc(-c3scnc3C)cc2)C(C)C)cn1]

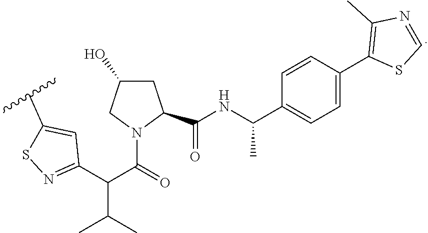 [SMILES: *c1cc(C(C(=O)N2C[C@H](O)C[C@H]2C(=O)N[C@@H](C)c2ccc(-c3scnc3C)cc2)C(C)C)ns1]

Embodiment 63. The compound Embodiment 39, or a pharmaceutically acceptable salt thereof, wherein the degradation moiety is

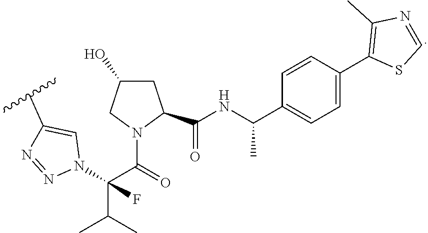

Embodiment 64. The compound of Embodiment 39, or a pharmaceutically acceptable salt thereof, wherein the degradation moiety is

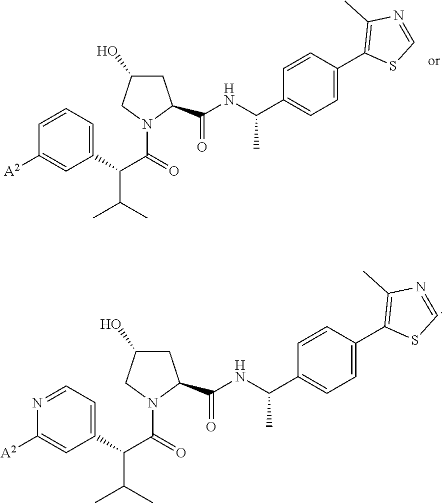

Embodiment 65. The compound of any one of Embodiments 1-24, or a pharmaceutically acceptable salt thereof, wherein the degradation moiety has the structure of Formula C5:

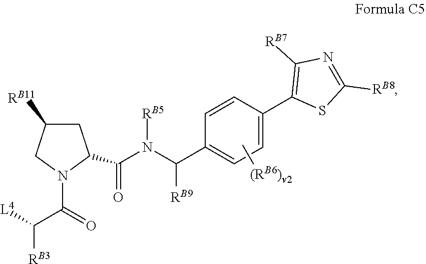

where
L⁴ is —N(R^{B1})(R^{B2}),

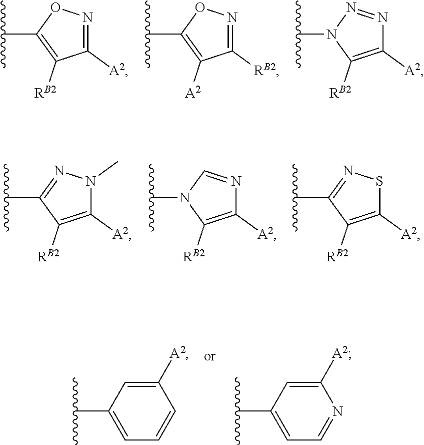

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₂-C₆ alkynyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl;
R^{B11} is H, alcohol, boronic acid, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
and
A² is a bond between the degradation moiety and the linker:
where one and only one of R^{B1}, R^{B3}, and R^{B6} is A²,
or a pharmaceutically acceptable salt thereof.

Embodiment 66. The compound of Embodiment 65, or a pharmaceutically acceptable salt thereof, wherein R^{B11} is boric acid.

Embodiment 67. The compound of any one of Embodiments 65-66, or a pharmaceutically acceptable salt thereof, wherein, the degradation moiety has the structure of Formula C6, Formula C7 or Formula C8.

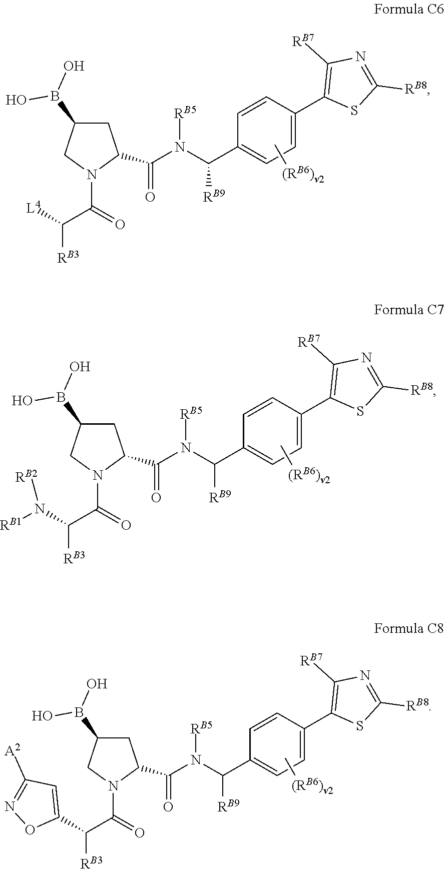

Embodiment 68. The compound of any one of Embodiments 65-66, or a pharmaceutically acceptable salt thereof, wherein R^{B9} is optionally substituted C₁-C₆ alkyl.

Embodiment 69. The compound of Embodiment 68, or a pharmaceutically acceptable salt thereof, wherein R^{B9} is methyl.

Embodiment 70. The compound of any one of Embodiments 65-69, or a pharmaceutically acceptable salt thereof, wherein R^{B9} is bonded to (S)-stereogenic center.

Embodiment 71. The compound of any one of Embodiments 65-70, or a pharmaceutically acceptable salt thereof, wherein v2 is 0.

Embodiment 72. The compound of any one of Embodiments 65-71, or a pharmaceutically acceptable salt thereof, wherein R^{B5} is H.

Embodiment 73. The compound of any one of Embodiments 65-72, or a pharmaceutically acceptable salt thereof, wherein R^{B7} is optionally substituted C₁-C₆ alkyl.

Embodiment 74. The compound of Embodiment 73, or a pharmaceutically acceptable salt thereof, wherein In some Embodiments, R^{B7} is methyl.

Embodiment 75. The compound of any one of Embodiments 65-74, or a pharmaceutically acceptable salt thereof, wherein, R^{B3} is optionally substituted C₁-C₆ alkyl.

Embodiment 76. The compound of Embodiment 75, or a pharmaceutically acceptable salt thereof, wherein R^{B3} is isopropyl.

Embodiment 77. The compound of any one of Embodiments 65-76, or a pharmaceutically acceptable salt thereof, wherein R^{B8} is H.

Embodiment 78. The compound of any one of Embodiments 65-77, or a pharmaceutically acceptable salt thereof, wherein, R^{B2} is H.

Embodiment 79. The compound of any one of Embodiments 65 and 664, or a pharmaceutically acceptable salt thereof, wherein the degradation moiety is

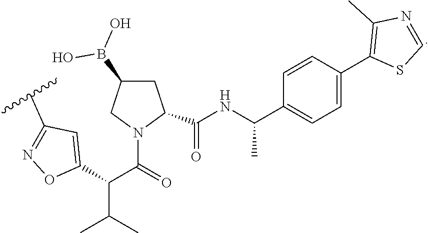 [SMILES: *c1cc([C@H](C(=O)N2C[C@@H](B(O)O)C[C@@H]2C(=O)N[C@@H](C)c2ccc(-c3scnc3C)cc2)C(C)C)on1]

Embodiment 80. The compound of any one of Embodiments 1 to 24, or a pharmaceutically acceptable salt thereof, wherein the degradation moiety has the structure of Formula D:

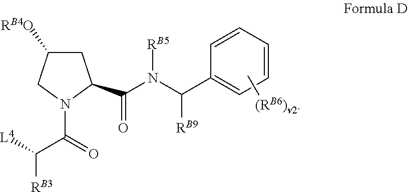

where
L⁴ is —N(R^{B1})(R^{B2}),

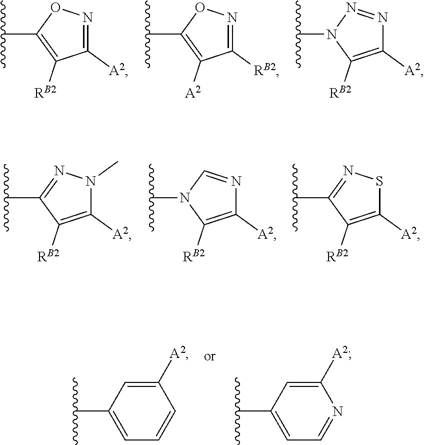

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A2, optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₂-C₆ alkynyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
R^{B9} is H or optionally substituted C₁-C₆ alkyl; and
A² is a bond between the degradation moiety and the linker;
where one and only one of R^{B1}, R^{B3}, and R^{B6} is A²,
or a pharmaceutically acceptable salt thereof.

Embodiment 81. The compound of Embodiment 80, or a pharmaceutically acceptable salt thereof, wherein the degradation moiety has the structure of Formula D3 or Formula D1:

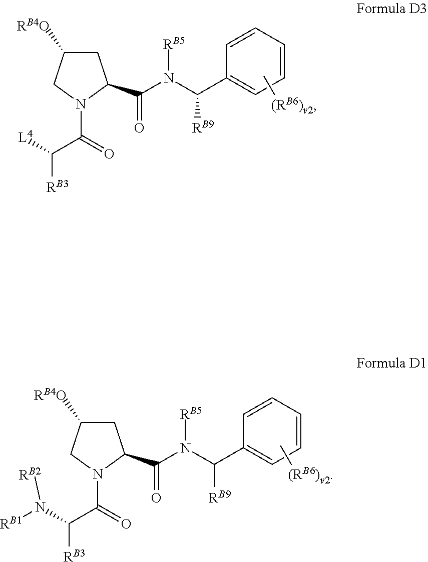

Embodiment 82. The compound of any one of Embodiments 80-81, or a pharmaceutically acceptable salt thereof, wherein the degradation moiety is

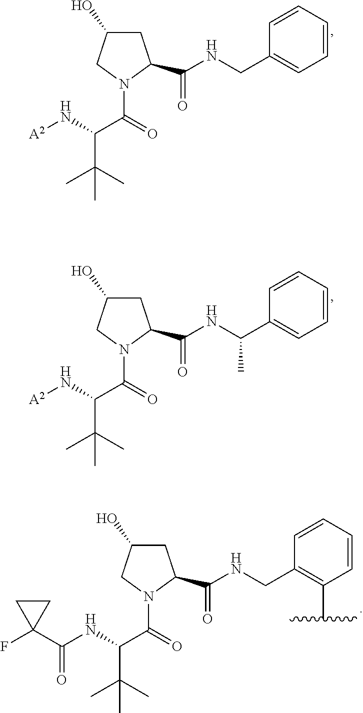

Embodiment 83. The compound of Embodiment 80, or a pharmaceutically acceptable salt thereof, wherein the degradation moiety has the structure of Formula D2:

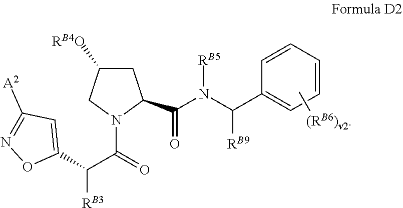

Embodiment 84. The compound of any one of Embodiments 80 and 83, or a pharmaceutically acceptable salt thereof, wherein R^{B9} is optionally substituted C₁-C₆ alkyl.

Embodiment 85. The compound of Embodiment 84, or a pharmaceutically acceptable salt thereof, wherein R^{B9} is methyl.

Embodiment 86. The compound of any one of Embodiments 80 and 83-85, or a pharmaceutically acceptable salt thereof, wherein R^{B9} is bonded to (S)-stereogenic center.

Embodiment 87. The compound of any one of Embodiments 80 and 83, or a pharmaceutically acceptable salt thereof, wherein R^{B9} is H.

Embodiment 88. The compound of any one of Embodiments 80 and 83-87, or a pharmaceutically acceptable salt thereof, wherein, v2 is 0.

Embodiment 89. The compound of any one of Embodiments 80 and 83-87, or a pharmaceutically acceptable salt thereof, wherein v2 is 1.

Embodiment 90. The compound of any one of Embodiments 80 and 83-87, or a pharmaceutically acceptable salt thereof, wherein v2 is 2.

Embodiment 91. The compound of any one of Embodiments 80 and 83-90, or a pharmaceutically acceptable salt thereof, wherein R^{B4} is H.

Embodiment 92. The compound of any one of Embodiments 80 and 83-91, or a pharmaceutically acceptable salt thereof, wherein R^{B5} is H.

Embodiment 93. The compound of any one of Embodiments 80 and 83-92, or a pharmaceutically acceptable salt thereof, wherein R^{B3} is optionally substituted C₁-C₆ alkyl.

Embodiment 94. The compound of Embodiment 93, or a pharmaceutically acceptable salt thereof, wherein R^{B3} is isopropyl.

Embodiment 95. The compound of any one of Embodiments 80 and 83-94, or a pharmaceutically acceptable salt thereof, wherein R^{B6} is H.

Embodiment 96. The compound of any one of Embodiments 80 and 83-94, or a pharmaceutically acceptable salt thereof, wherein R^{B6} is fluorine, chlorine or bromine.

Embodiment 97. The compound of any one of Embodiments 80 and 83-94, or a pharmaceutically acceptable salt thereof, wherein R^{B6} is cyano.

Embodiment 98. The compound of any one of Embodiments 80 and 83-94, or a pharmaceutically acceptable salt thereof, wherein R^{B6} is optionally substituted C₁-C₆ heteroalkyl.

Embodiment 99. The compound of Embodiment 98, or a pharmaceutically acceptable salt thereof, wherein R^{B6} is methoxy or 3-methoxy-1-propanoxy.

Embodiment 100. The compound of any one of Embodiments 80 and 83-94, or a pharmaceutically acceptable salt thereof, wherein R^{B6} is optionally substituted C₃-C₆ alkynyl.

Embodiment 101. The compound of Embodiment 80, or a pharmaceutically acceptable salt thereof, wherein the degradation moiety is

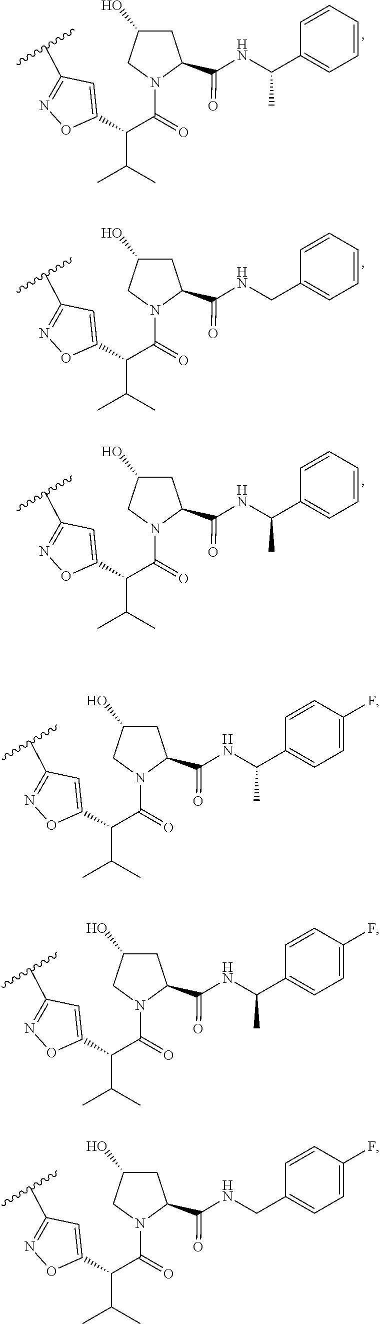

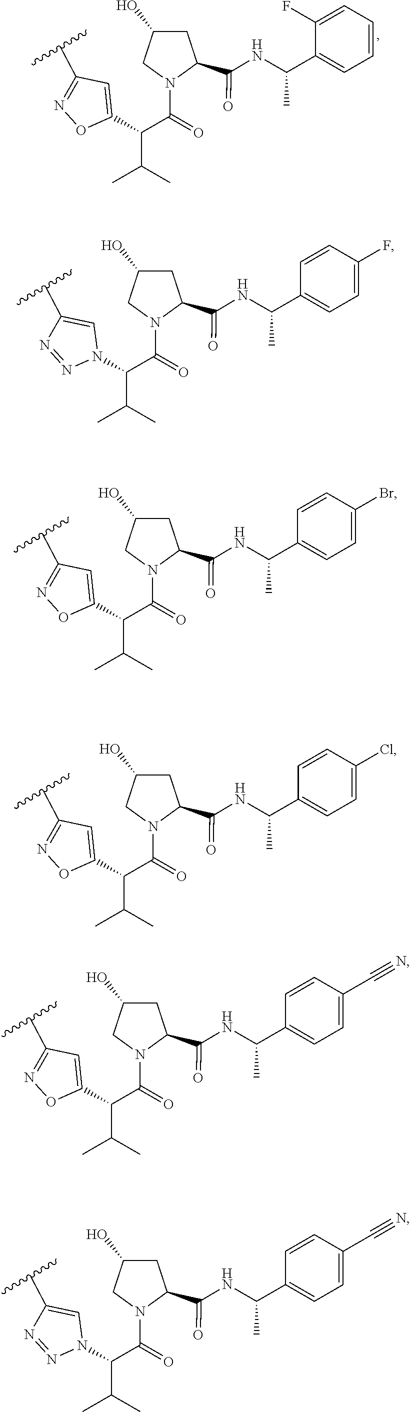 [SMILES: *c1cc([C@H](C(=O)N2C[C@H](O)C[C@H]2C(=O)N[C@@H](C)c2ccccc2F)C(C)C)on1 ; *c1cn([C@H](C(=O)N2C[C@H](O)C[C@H]2C(=O)N[C@@H](C)c2ccc(F)cc2)C(C)C)nn1 ; *c1cc([C@H](C(=O)N2C[C@H](O)C[C@H]2C(=O)N[C@@H](C)c2ccc(Br)cc2)C(C)C)on1 ; *c1cc([C@H](C(=O)N2C[C@H](O)C[C@H]2C(=O)N[C@@H](C)c2ccc(Cl)cc2)C(C)C)on1 ; *c1cc([C@H](C(=O)N2C[C@H](O)C[C@H]2C(=O)N[C@@H](C)c2ccc(C#N)cc2)C(C)C)on1 ; *c1cn([C@H](C(=O)N2C[C@H](O)C[C@H]2C(=O)N[C@@H](C)c2ccc(C#N)cc2)C(C)C)nn1]

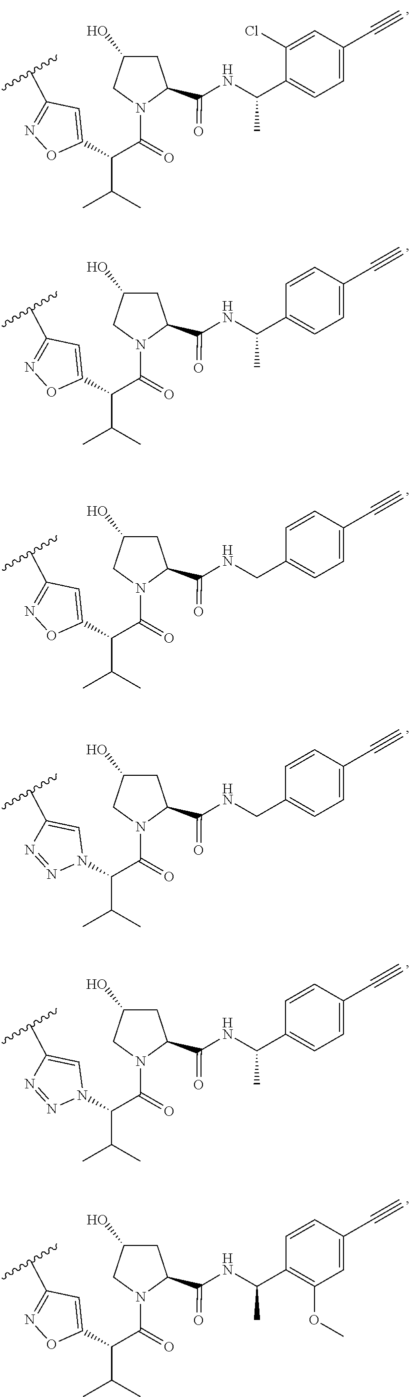 [SMILES: *c1cc([C@H](C(=O)N2C[C@H](O)C[C@H]2C(=O)N[C@@H](C)c2ccc(C#C)cc2Cl)C(C)C)on1 ; *c1cc([C@H](C(=O)N2C[C@H](O)C[C@H]2C(=O)N[C@@H](C)c2ccc(C#C)cc2)C(C)C)on1 ; *c1cc([C@H](C(=O)N2C[C@H](O)C[C@H]2C(=O)NCc2ccc(C#C)cc2)C(C)C)on1 ; *c1cn([C@H](C(=O)N2C[C@H](O)C[C@H]2C(=O)NCc2ccc(C#C)cc2)C(C)C)nn1 ; *c1cn([C@H](C(=O)N2C[C@H](O)C[C@H]2C(=O)N[C@@H](C)c2ccc(C#C)cc2)C(C)C)nn1 ; *c1cc([C@H](C(=O)N2C[C@H](O)C[C@H]2C(=O)N[C@H](C)c2ccc(C#C)cc2OC)C(C)C)on1]

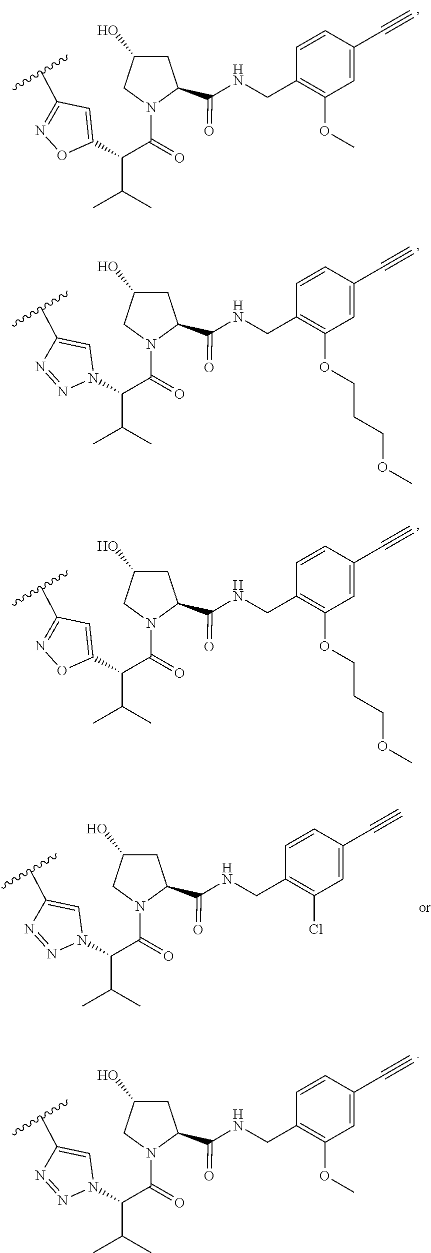

Embodiment 102. The compound of any one of Embodiments 1-24, or a pharmaceutically acceptable salt thereof, wherein the degradation moiety has the structure of Formula Da:

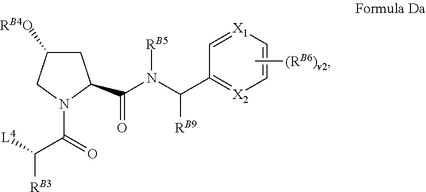

where
L⁴ is —N(R^{B1})(R^{B2}),

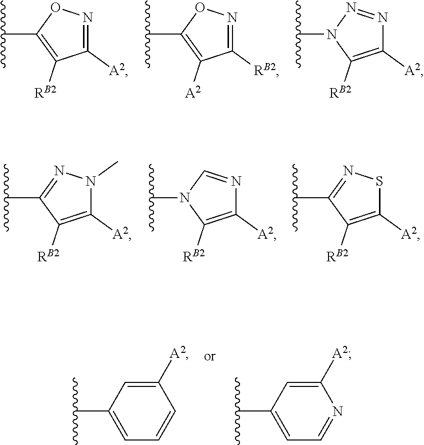

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
Each of X₁ and X₂ are, independently, C, N, or O.
v2 is 0, 1, 2, 3, or 4;
each R^{B6} is, independently, A², halogen, optionally substituted C₁-C₆ alkyl, optionally substituted C₂-C₆ alkynyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, cyano, or optionally substituted amino;
R^{B9} is H or optionally substituted C₁-C₆ alkyl; and
A² is a bond between the degradation moiety and the linker;
where one and only one of R^{B1}, R^{B3}, and R^{B6} is A²,
or a pharmaceutically acceptable salt thereof.

Embodiment 103. The compound of Embodiment 102, or a pharmaceutically acceptable salt thereof, wherein the degradation moiety has the structure of the degradation moiety has the structure of Formula Da3, Formula Dal or Formula Da2.

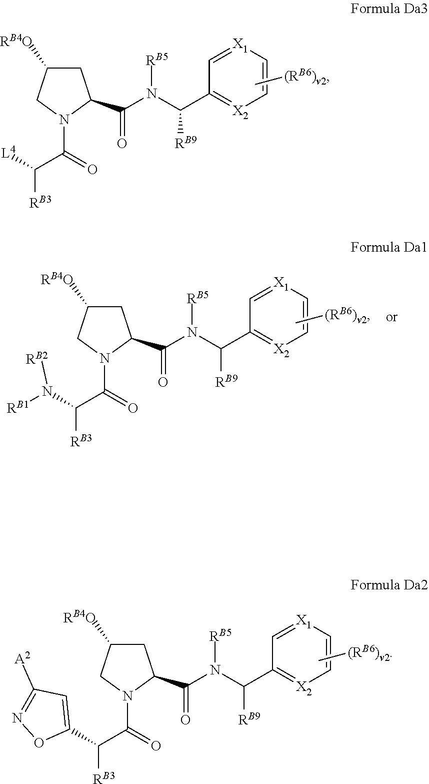

Embodiment 104. The compound of any one of Embodiments 102-103, or a pharmaceutically acceptable salt thereof, wherein R^{B9} is optionally substituted C₁-C₆ alkyl.

Embodiment 105. The compound of Embodiment 104, or a pharmaceutically acceptable salt thereof, wherein R^{B9} is methyl.

Embodiment 106. The compound of any one of Embodiments 102-105, or a pharmaceutically acceptable salt thereof, wherein R^{B9} is bonded to (S)-stereogenic center.

Embodiment 107. The compound of any one of Embodiments 102-106, or a pharmaceutically acceptable salt thereof, wherein v2 is 0.

Embodiment 108. The compound of any one of Embodiments 102-107, or a pharmaceutically acceptable salt thereof, wherein R^{B4} is H.

Embodiment 109. The compound of any one of Embodiments 102-108, or a pharmaceutically acceptable salt thereof, wherein R^{B5} is H.

Embodiment 110. The compound of any one of Embodiments 102-109, or a pharmaceutically acceptable salt thereof, wherein R^{B3} is optionally substituted C₁-C₆ alkyl.

Embodiment 111. The compound of Embodiment 110, or a pharmaceutically acceptable salt thereof, wherein R^{B3} is isopropyl.

Embodiment 112. The compound of any one of Embodiments 102-111, or a pharmaceutically acceptable salt thereof, wherein X₁ is C and X₂ is N.

Embodiment 113. The compound of Embodiment 102, or a pharmaceutically acceptable salt thereof, wherein the degradation moiety is

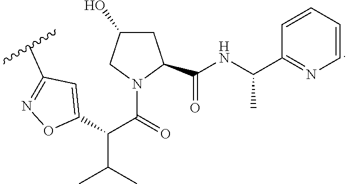 [SMILES: *c1cc([C@H](C(=O)N2C[C@H](O)C[C@H]2C(=O)N[C@@H](C)c2ccccn2)C(C)C)on1]

Embodiment 114. The compound of any one of Embodiments 1-24, or a pharmaceutically acceptable salt thereof, wherein the degradation moiety has the structure of Formula E:

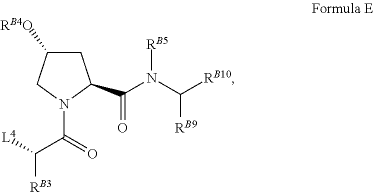

where
L⁴ is —N(R^{B1})(R^{B2}),

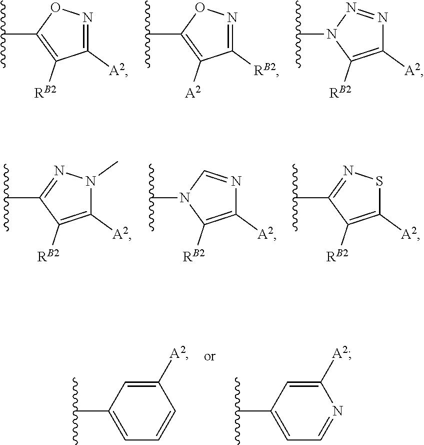

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
R^{B9} is H, optionally substituted C₁-C₆ alkyl, optionally substituted C₃-C₆ alkynyl, optionally substituted C₃-C₁₀ carbocyclyl, or optionally substituted C₂-C₁₀ heterocyclyl;
B¹⁰ is H, optionally substituted C₁-C₆ alkyl, optionally substituted C₃—C alkynyl, optionally substituted C₃-C₁₀ carbocyclyl, optionally substituted C₂-C₁₀ heterocyclyl; optionally substituted amino, or cyano, and
A² is a bond between the degradation moiety and the linker;
where one and only one of R^{B1}, R^{B3}, and R^{B6} is A²,
or a pharmaceutically acceptable salt thereof.

Embodiment 115. The compound of Embodiment 114, or a pharmaceutically acceptable salt thereof, wherein the degradation moiety has the structure of Formula E3 or Formula E1.

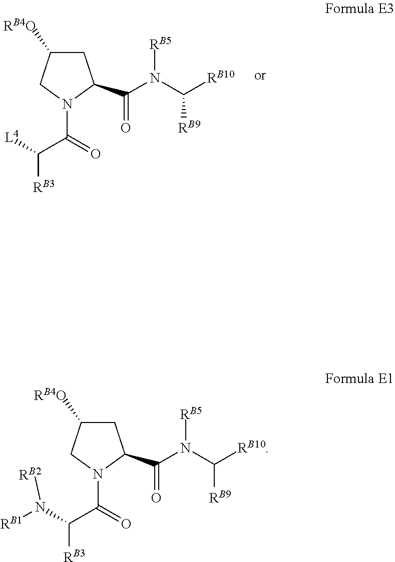

Embodiment 116. The compound of Embodiment 114, or a pharmaceutically acceptable salt thereof, wherein the degradation moiety is

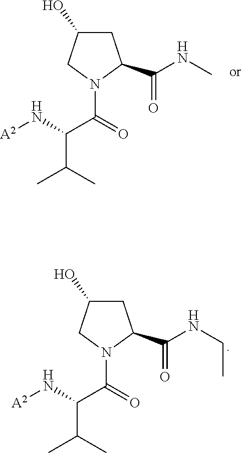

Embodiment 117. The compound of Embodiment 114, or a pharmaceutically acceptable salt thereof, wherein, the degradation moiety has the structure of Formula E2:

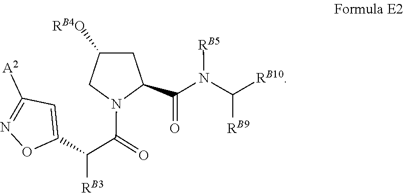

Embodiment 118. The compound of of Embodiment 114-115, or a pharmaceutically acceptable salt thereof, wherein R^{B9} is optionally substituted C₁-C₆ alkyl.

Embodiment 119. The compound of Embodiment 118, or a pharmaceutically acceptable salt thereof, wherein R^{B9} is methyl.

Embodiment 120. The compound of any one of Embodiments 114-115, or a pharmaceutically acceptable salt thereof, wherein R^{B9} is optionally substituted C₃-C₆ alkynyl.

Embodiment 121. The compound of any one of Embodiments 114-115, or a pharmaceutically acceptable salt thereof, wherein R^{B9} is [1.1.1]pentane, cyclopropane, cyclobutene or cyclopentane.

Embodiment 122. The compound of any one of Embodiments 114-115 and 117-121, or a pharmaceutically acceptable salt thereof, wherein R^{B9} is bonded to (S)-stereogenic center.

Embodiment 123. The compound of any one of Embodiments 114-115, or a pharmaceutically acceptable salt thereof, wherein R^{B9} is H.

Embodiment 124. The compound of any one of Embodiments 114-115 and 117-123, or a pharmaceutically acceptable salt thereof, wherein R^{B4} is H.

Embodiment 125. The compound of any one of Embodiments 114-115 and 117-124, or a pharmaceutically acceptable salt thereof, wherein R^{B5} is H.

Embodiment 126. The compound of any one of Embodiments 114-115 and 117-125, or a pharmaceutically acceptable salt thereof, wherein R^{B3} is optionally substituted C₁-C₆ alkyl.

Embodiment 127. The compound of Embodiment 126, or a pharmaceutically acceptable salt thereof, wherein R^{B3} is isopropyl.

Embodiment 128. The compound of any one of Embodiments 114-115 and 117-127, or a pharmaceutically acceptable salt thereof, wherein R^{B2} is H.

Embodiment 129. The compound of any one of Embodiments 114-115 and 117-1285, or a pharmaceutically acceptable salt thereof, wherein R^{B10} is absent.

Embodiment 130. The compound of any one of Embodiments 114-115 and 117-128, or a pharmaceutically acceptable salt thereof, wherein R^{B10} is H or cyano.

Embodiment 131. The compound of any one of Embodiments 114-115 and 117-128, or a pharmaceutically acceptable salt thereof, wherein R^{B10} is optionally substituted C₃-C₁₀ carbocyclyl,

Embodiment 132. The compound of any one of Embodiments 114-115 and 117-128, or a pharmaceutically acceptable salt thereof, wherein R^{B10} is optionally substituted C₁-C₆ alkyl.

Embodiment 133. The compound of Embodiment 132, or a pharmaceutically acceptable salt thereof, wherein R^{B10} is methyl.

Embodiment 134. The compound of Embodiment 114, or a pharmaceutically acceptable salt thereof, wherein the degradation moiety is

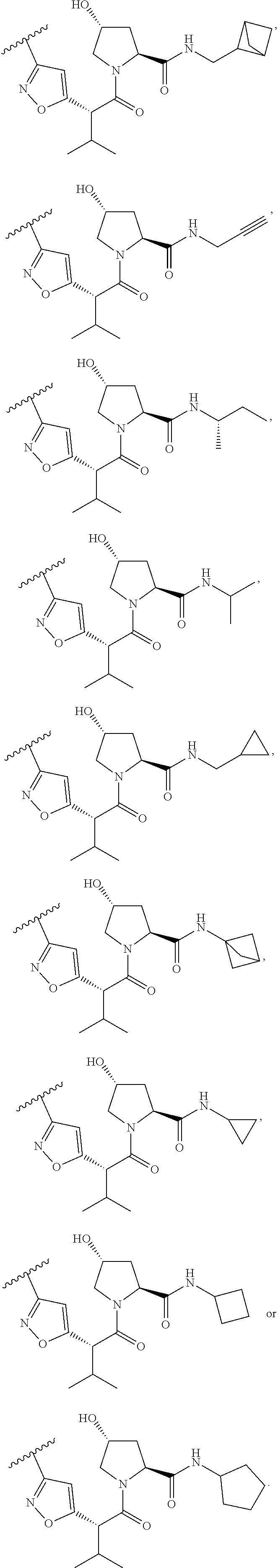

Embodiment 135. The compound of any one of Embodiments 1-24, or a pharmaceutically acceptable salt thereof, wherein the degradation moiety has the structure of Formula F:

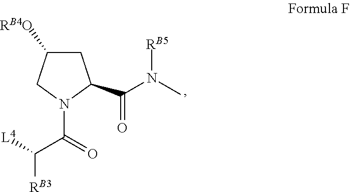

where
L⁴ is —N(R^{B1})(R^{B2}),

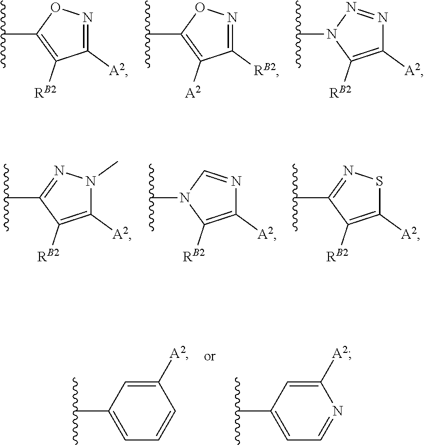

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
A² is a bond between the degradation moiety and the linker;
where one and only one of R^{B1} or R^{B3} is A²,
or a pharmaceutically acceptable salt thereof.

Embodiment 136. The compound of Embodiment 135, or a pharmaceutically acceptable salt thereof, wherein the degradation moiety has the structure of Formula F3 or Formula F1.

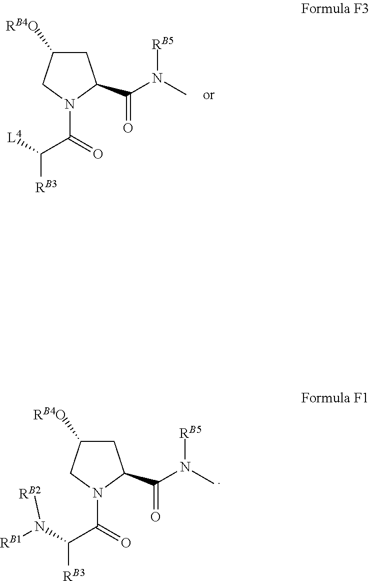

Embodiment 137. The compound of Embodiment 135, or a pharmaceutically acceptable salt thereof, wherein the degradation moiety is

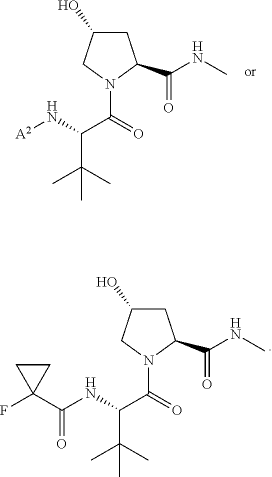

Embodiment 138. The compound of Embodiment 135, or a pharmaceutically acceptable salt thereof, wherein the degradation moiety has the structure of Formula F2:

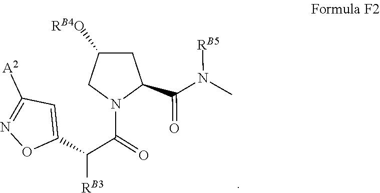

Embodiment 139. The compound of any one of Embodiments 135 and 138, or a pharmaceutically acceptable salt thereof, wherein R^{B9} is optionally substituted C₁-C₆ alkyl.

Embodiment 140. The compound of Embodiment 139, or a pharmaceutically acceptable salt thereof, wherein R^{B9} is methyl.

Embodiment 141. The compound of any one of Embodiments 135 and 138-140, or a pharmaceutically acceptable salt thereof, wherein R^{B4} is H.

Embodiment 142. The compound of any one of Embodiments 135 and 138-141, or a pharmaceutically acceptable salt thereof, wherein R^{B5} is H.

Embodiment 143. The compound of any one of Embodiments 135 and 138-142, or a pharmaceutically acceptable salt thereof, wherein R^{B3} is optionally substituted C₁-C₆ alkyl.

Embodiment 144. The compound of Embodiment 143, or a pharmaceutically acceptable salt thereof, wherein R^{B3} is isopropyl.

Embodiment 145. The compound of any one of Embodiments 135 and 138-144, or a pharmaceutically acceptable salt thereof, wherein R^{B2} is H.

Embodiment 146. The compound of Embodiment 135, or a pharmaceutically acceptable salt thereof, wherein the degradation moiety is

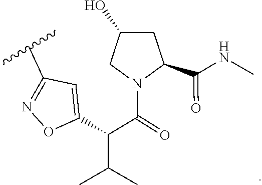 [SMILES: *c1cc([C@H](C(=O)N2C[C@H](O)C[C@H]2C(=O)NC)C(C)C)on1]

Embodiment 147. The compound of any one of Embodiments 1 to 146, or a pharmaceutically acceptable salt thereof, wherein the linker has the structure of Formula II:

A¹-(B¹)_{f}-(C¹)_{g}-(B²)ₕ-(D)-(B³)ᵢ-(C²)ⱼ-(B⁴)ₖ-A², Formula II
or a pharmaceutically acceptable salt thereof,
wherein
A¹ is a bond between the linker and the ring system A;
A² is a bond between the degradation moiety and the linker;
each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₃-C₁₀ carbocyclyl, optionally substituted C₂-C₁₀ heterocyclyl, optionally substituted C₂-C₆ heteroaryl, optionally substituted C₆₋₁₂ aryl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₁₀ heterocyclyl, optionally substituted C₂₋₆ heteroaryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₁₀ heterocyclyl, optionally substituted C₂₋₆ heteroaryl, optionally substituted C₆₋₁₂ aryl, optionally substituted C₂-C₁₀ polyethylene glycol, optionally substituted C₃-C₁₀ cycloalkyl, optionally substituted C₃-C₁₀ carbocyclyl, or optionally substituted C₁₋₁₀ heteroalkyl; or D is absent, and the linker is A¹-(B¹)_{f}-(C¹)_{g}-(B²)ₕ-(B³)ᵢ-(C²)ⱼ-(B⁴)ₖ-A².

Embodiment 148. The compound of Embodiment 147, or a pharmaceutically acceptable salt thereof, wherein
A¹ is a bond between the linker and the benzopyridazine core ring system;
A² is a bond between the degradation moiety and the linker;
each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₂-C₈ heterocyclyl, optionally substituted C₂-C₆ heteroaryl, optionally substituted C₆₋₁₂ aryl, O, S, S(O)₂, or NRN;
each RN is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₂₋₆ heteroaryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₂₋₆ heteroaryl, optionally substituted C₆₋₁₂ aryl, optionally substituted C₂-C₁₀ polyethylene glycol, or optionally substituted C₁₋₁₀ heteroalkyl; or D is absent, and the linker is A¹-(B¹)_{f}-(C¹)_{g}-(B²)ₕ-(B³)ᵢ—(C²)ⱼ-(B⁴)ₖ-A².

Embodiment 149. The compound of any one of Embodiments 147-148, or a pharmaceutically acceptable salt thereof, wherein each of B¹, B², B³, and B⁴ is, independently, optionally substituted C₁-C₂ alkyl, optionally substituted C₁-C₃ heteroalkyl, optionally substituted C₂-C₁₀ heterocyclyl, optionally substituted C₂₋₆ heteroaryl, O, or NR^{N}.

Embodiment 150. The compound of any one of Embodiments 147-148, or a pharmaceutically acceptable salt thereof, wherein each of B¹, B², B³, and B⁴ is, independently, optionally substituted C₁-C₂ alkyl, optionally substituted C₁-C₃ heteroalkyl, optionally substituted C₂-C₈ heterocyclyl, optionally substituted C₂₋₆ heteroaryl, or O.

Embodiment 151. The compound of any one of Embodiments 147-150, or a pharmaceutically acceptable salt thereof, wherein each of B¹ and B⁴ is, independently,

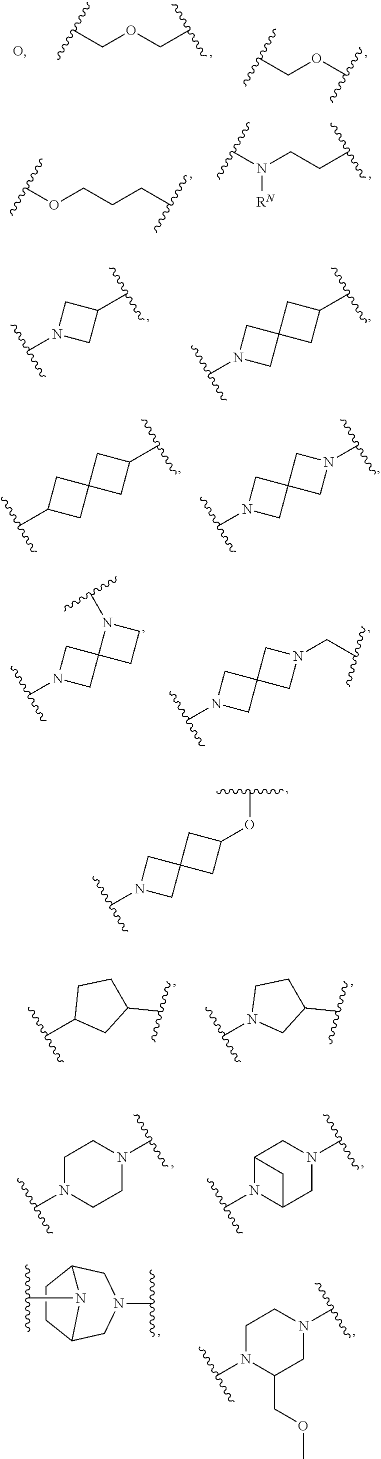

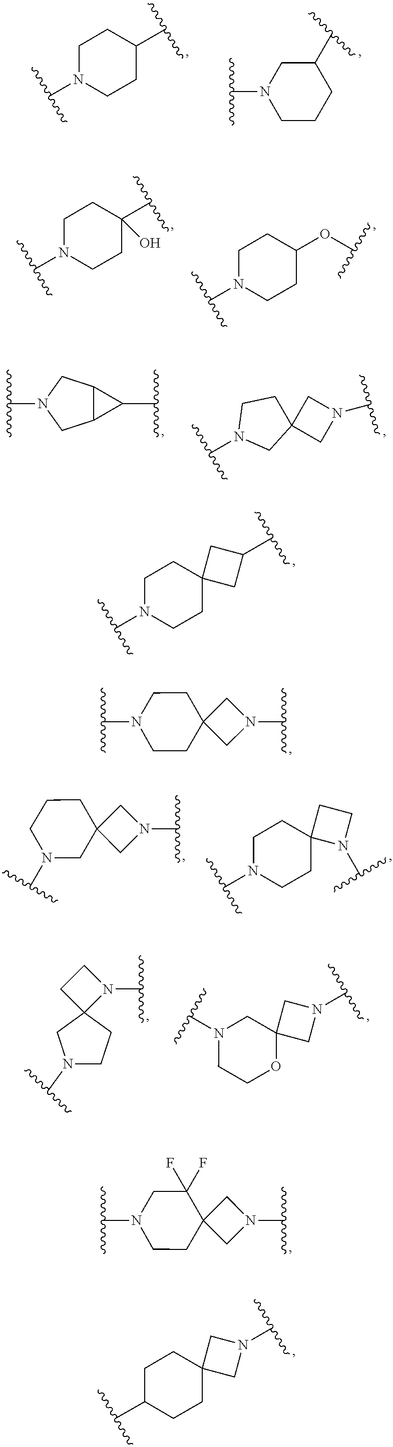

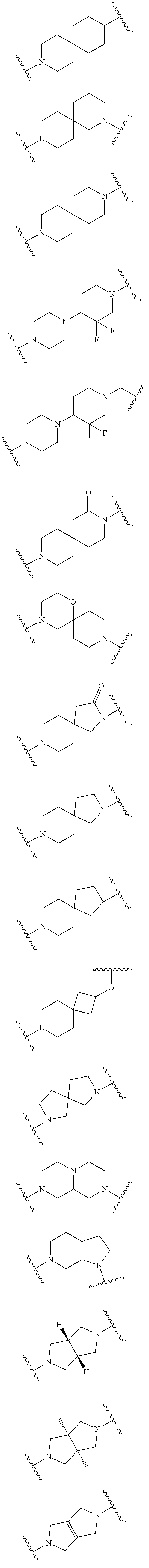

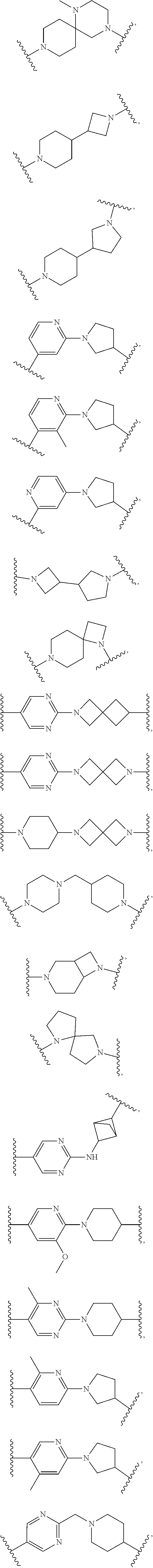

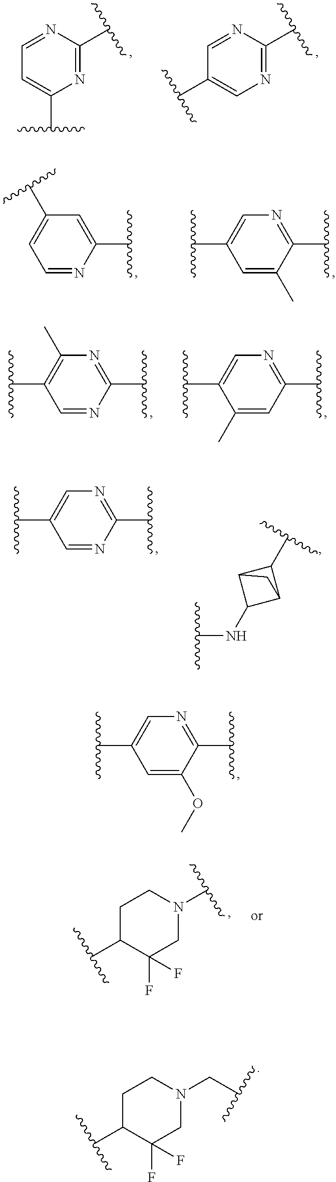

Embodiment 152. The compound of any one of Embodiments 147-150, or a pharmaceutically acceptable salt thereof, wherein each of B¹ and B⁴ is, independently,

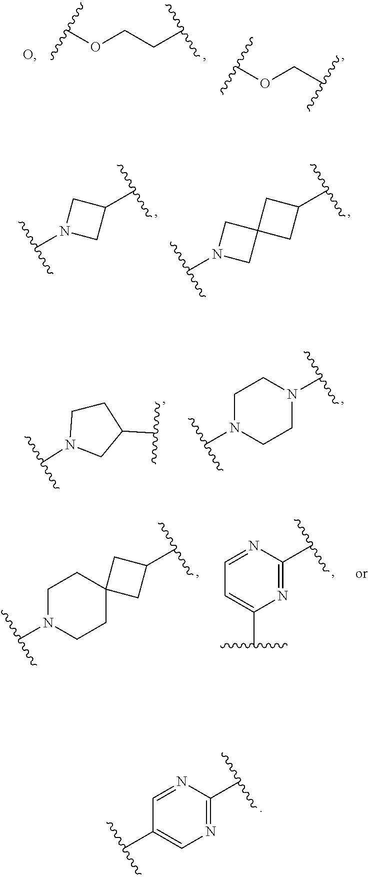

Embodiment 153. The compound of any one of Embodiments 147-151, or a pharmaceutically acceptable salt thereof, wherein B¹ is

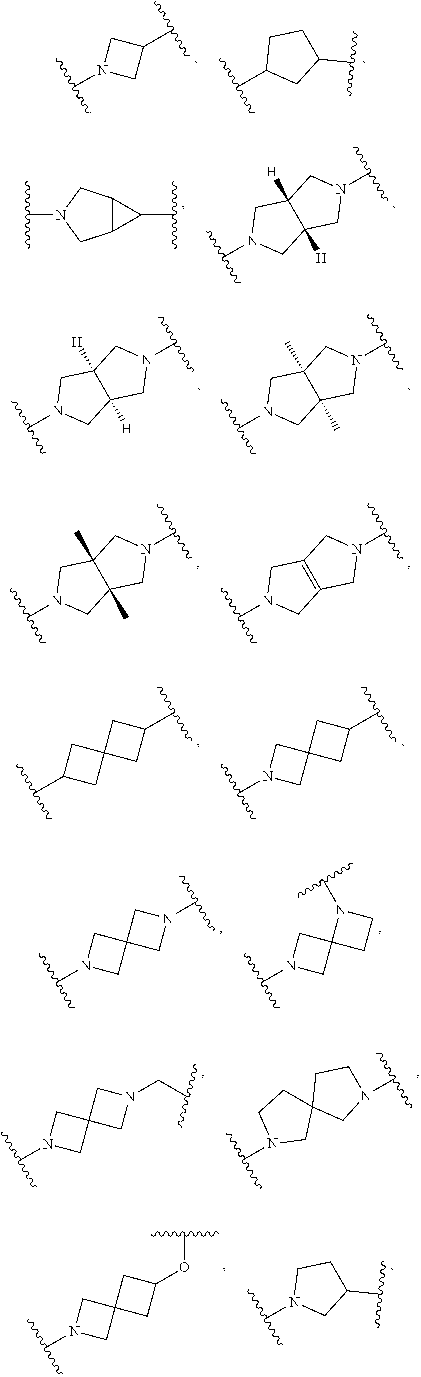

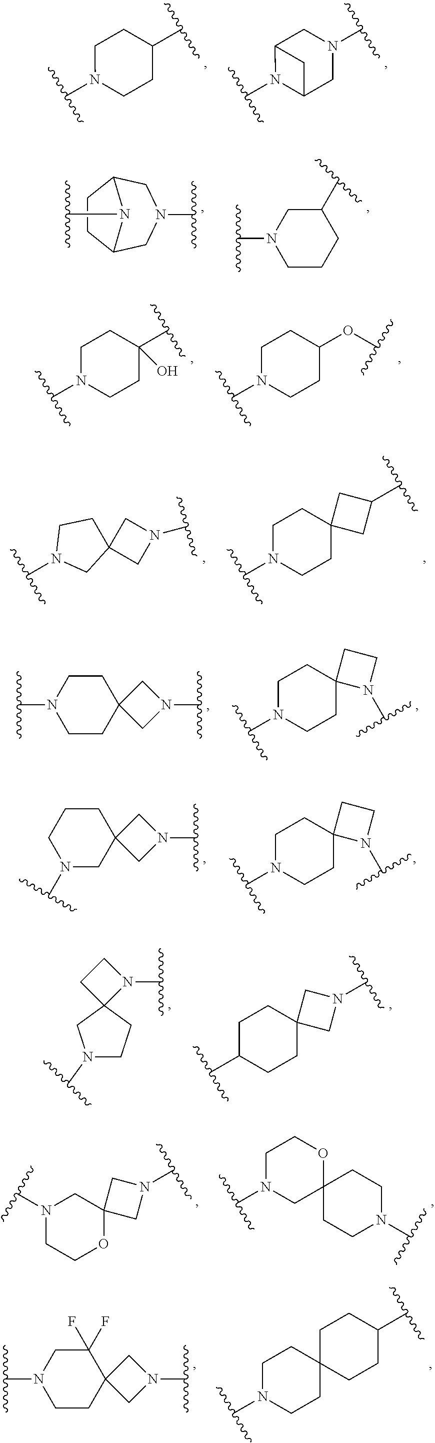

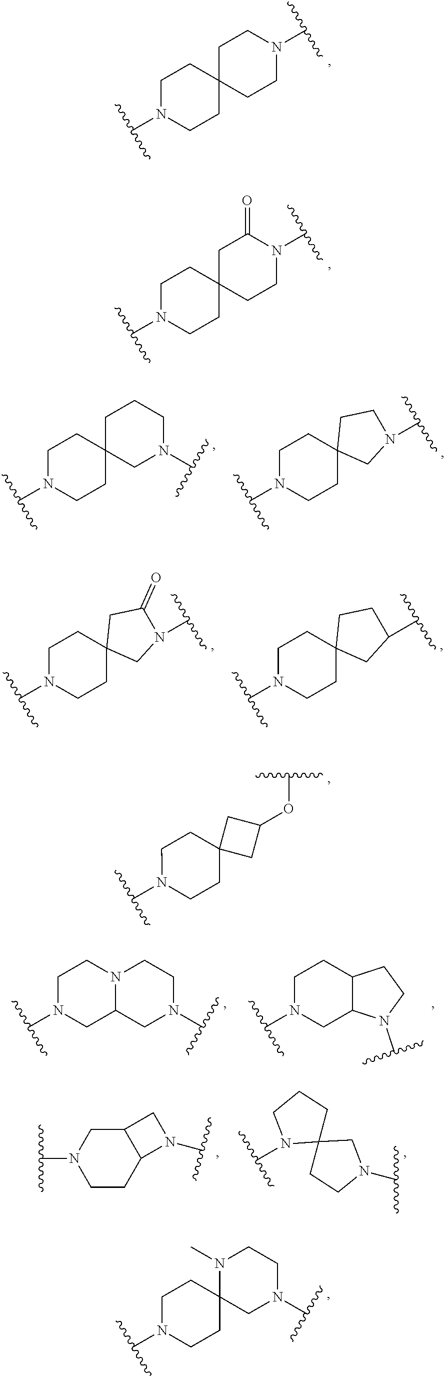

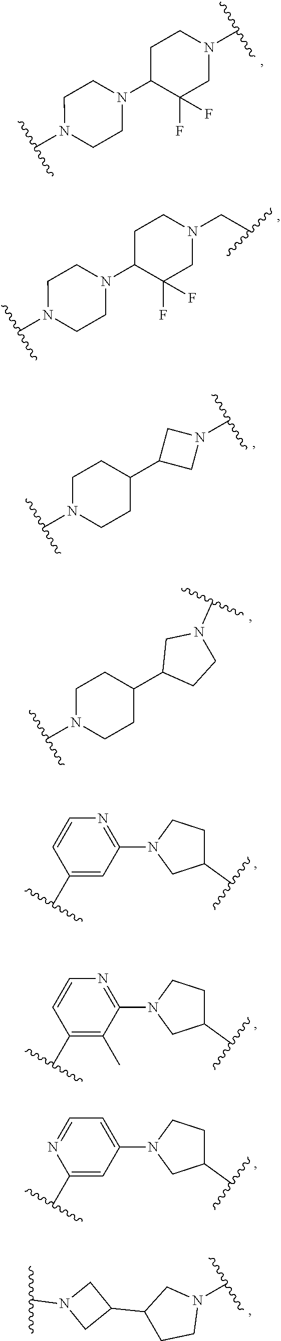

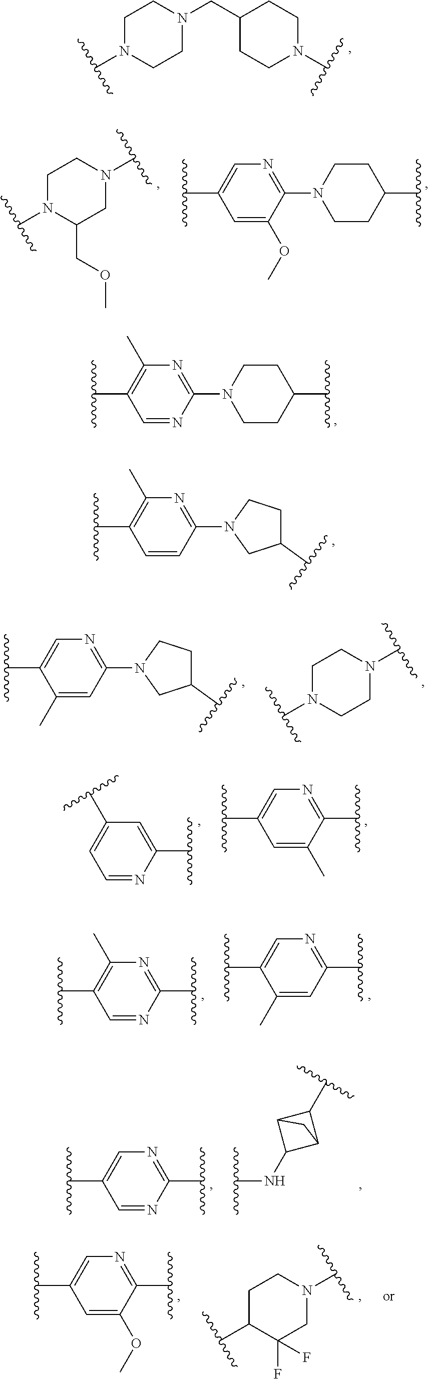

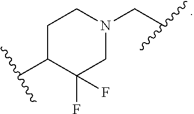 [SMILES: *CN1CCC(*)C(F)(F)C1]

Embodiment 154. The compound of any one of Embodiments 147-151 and 153, or a pharmaceutically acceptable salt thereof, wherein B⁴ is

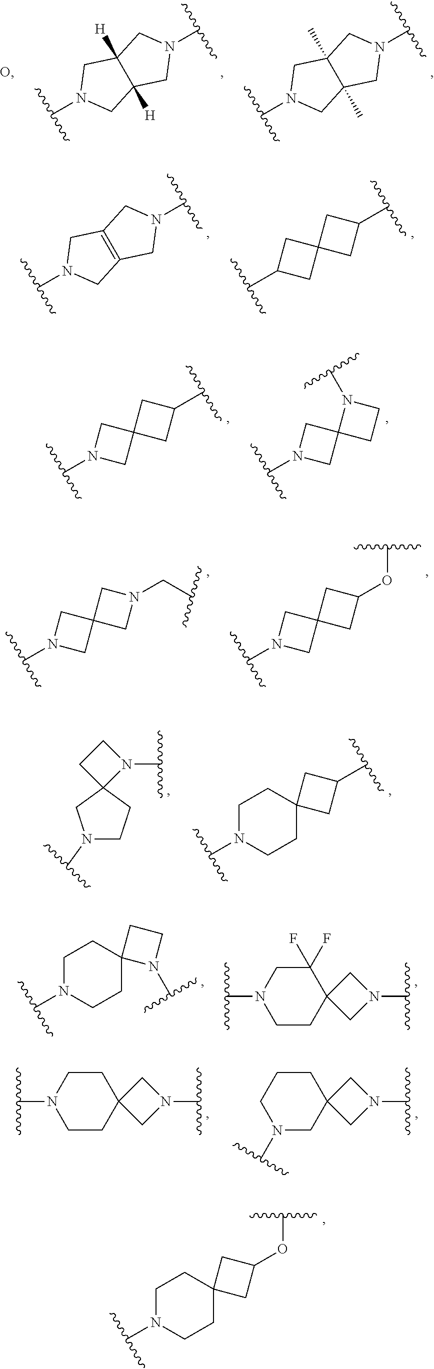

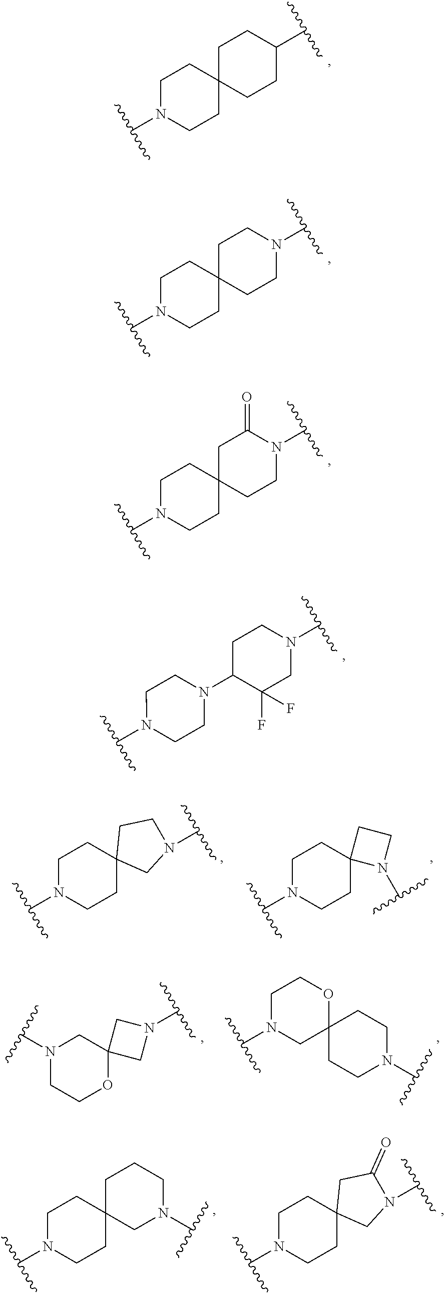

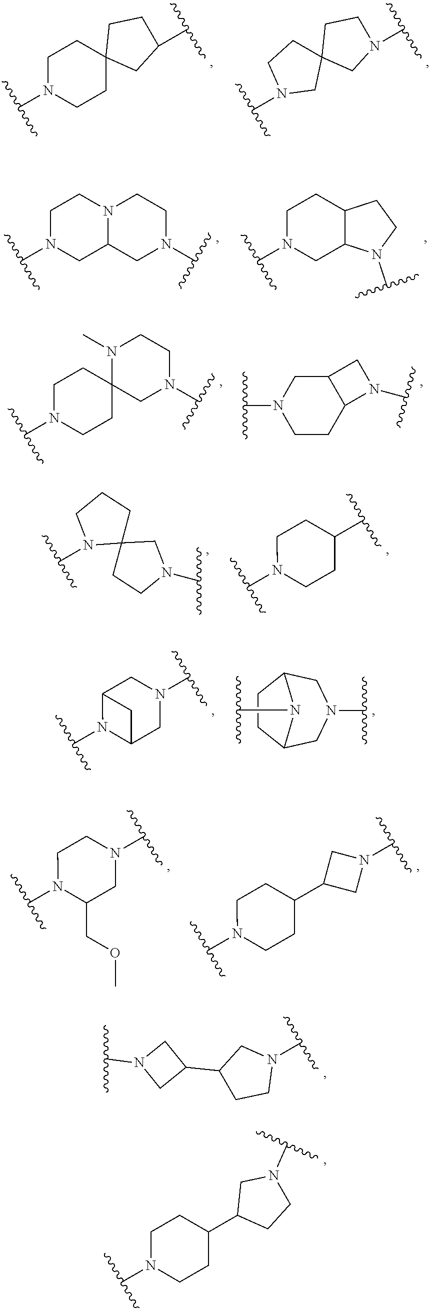

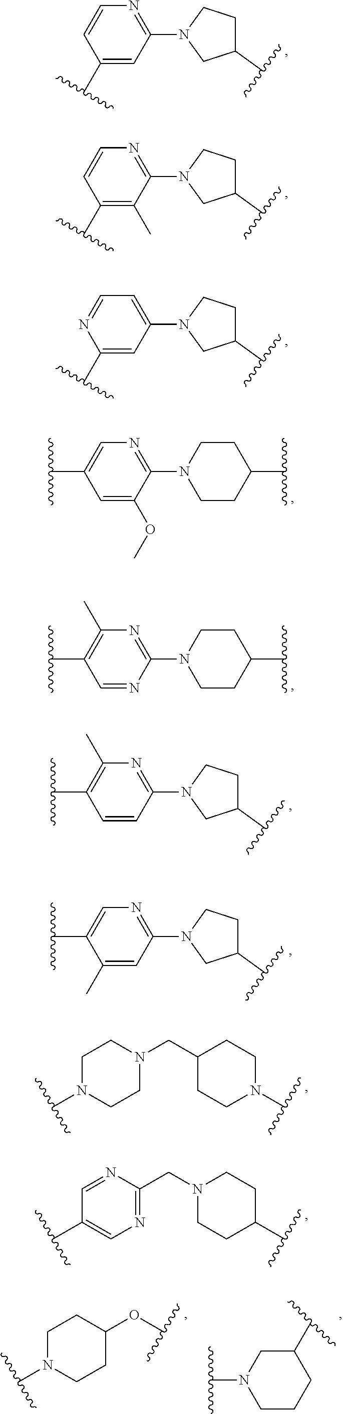

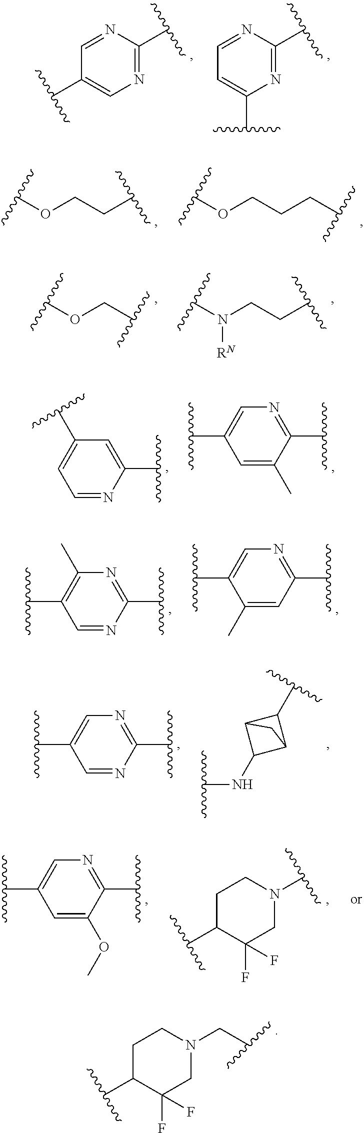

Embodiment 155. The compound of any one of Embodiments 147 to 154, or a pharmaceutically acceptable salt thereof, wherein C¹ is

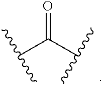 [SMILES: *C(*)=O]

Embodiment 156. The compound of any one of Embodiments 147 to 155, or a pharmaceutically acceptable salt thereof, wherein B² is optionally substituted C₁-C₄ alkyl.

Embodiment 157. The compound of any one of Embodiments 147 to 156, or a pharmaceutically acceptable salt thereof, wherein D is optionally substituted C₁-C₁₀ alkyl.

Embodiment 158. The compound of any one of Embodiments 147 to 157, or a pharmaceutically acceptable salt thereof, wherein f is 1.

Embodiment 159. The compound of any one of Embodiments 147 to 158, or a pharmaceutically acceptable salt thereof, wherein g, h, l and j are 0.

Embodiment 160. The compound of any one of Embodiments 147 to 159, or a pharmaceutically acceptable salt thereof, wherein k is 0.

Embodiment 161. The compound of any one of Embodiments 147 to 159, or a pharmaceutically acceptable salt thereof, wherein k is 1.

Embodiment 162. The compound of any one of Embodiments 147-156 and 158-161, or a pharmaceutically acceptable salt thereof, wherein D is absent, and the linker is A¹-(B¹)_{f}-(C¹)_{g}—(B²)ₕ-(B³)ᵢ-(C²)ⱼ-(B⁴)ₖ-A².

Embodiment 163. The compound of any one of Embodiments 147-156 and 158-161, or a pharmaceutically acceptable salt thereof, wherein D is optionally substituted C₁₋₁₀ alkyl, optionally substituted C₂₋₁₀ alkenyl, optionally substituted C₂₋₁₀ alkynyl, optionally substituted C₂₋₁₀ heterocyclyl, optionally substituted C₂₋₆ heteroaryl, optionally substituted C₆₋₁₂ aryl, optionally substituted C₂-C₁₀ polyethylene glycol, or optionally substituted C₁₋₁₀ heteroalkyl.

Embodiment 164. The compound of any one of Embodiments 147-156 and 158-161, or a pharmaceutically acceptable salt thereof, wherein D is optionally substituted C₃-C₁₀ cycloalkyl, f is 1, g is 0, h is 0, i is 0, j is 0, and, k is 1.

Embodiment 165. The compound of any one of Embodiments 147-156 and 158-161, or a pharmaceutically acceptable salt thereof, wherein D is optionally substituted C₃-C₁₀ cycloalkyl, f is 1, g is 0, h is 0, i is 0, j is 0, and, k is 0.

Embodiment 166. The compound of any one of Embodiments 147-156 and 158-161, or a pharmaceutically acceptable salt thereof, wherein D is optionally substituted C₃-C₁₀ cycloalkyl, f is 0, g is 0, h is 0, i is 0, j is 0, and, k is 1.

Embodiment 167. The compound of any one of Embodiments 147-156 and 158-161, or a pharmaceutically acceptable salt thereof, wherein D is optionally substituted C₃-C₁₀ cycloalkyl, f is 0, g is 0, h is 0, i is 0, j is 0, and, k is 0.

Embodiment 168. The compound of any one of Embodiments 147-156 and 158-161, or a pharmaceutically acceptable salt thereof, wherein D is optionally substituted C₃-C₁₀ carbocyclyl, f is 1, g is 0, h is 0, i is 0, j is 0, and, k is 1.

Embodiment 169. The compound of any one of Embodiments 147-156 and 158-161, or a pharmaceutically acceptable salt thereof, wherein D is optionally substituted C₃-C₁₀ carbocyclyl, f is 1, g is 0, h is 0, 1 is 0, j is 0, and, k is 0.

Embodiment 170. The compound of any one of Embodiments 147-156 and 158-161, or a pharmaceutically acceptable salt thereof, wherein D is optionally substituted C₃-C₁₀ carbocyclyl, f is 0, g is 0, h is 0, i is 0, j is 0, and, k is 1.

Embodiment 171. The compound of any one of Embodiments 147-156 and 158-161, or a pharmaceutically acceptable salt thereof, wherein D is optionally substituted C₃-C₁₀ carbocyclyl, f is 0, g is 0, h is 0, i is 0, j is 0, and, k is 0.

Embodiment 172. The compound of any one of Embodiments 147-156 and 158-161, or a pharmaceutically acceptable salt thereof, wherein D is:

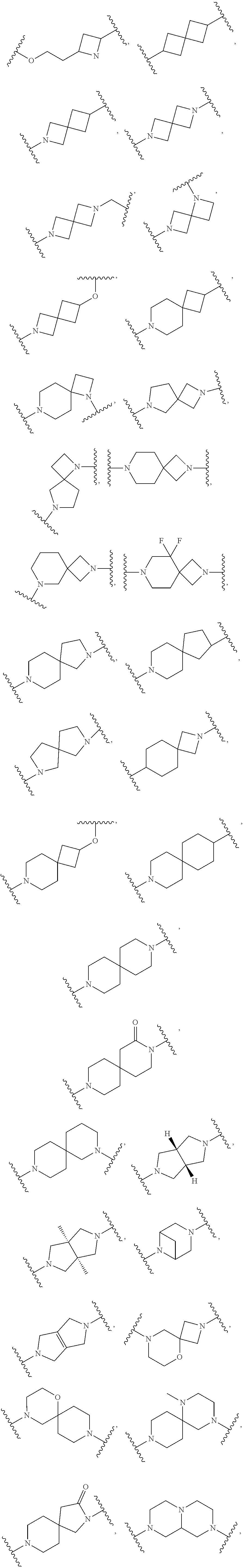

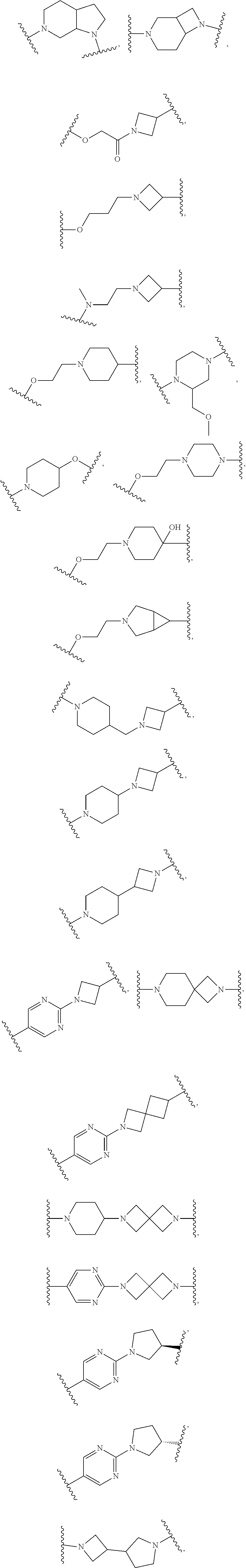

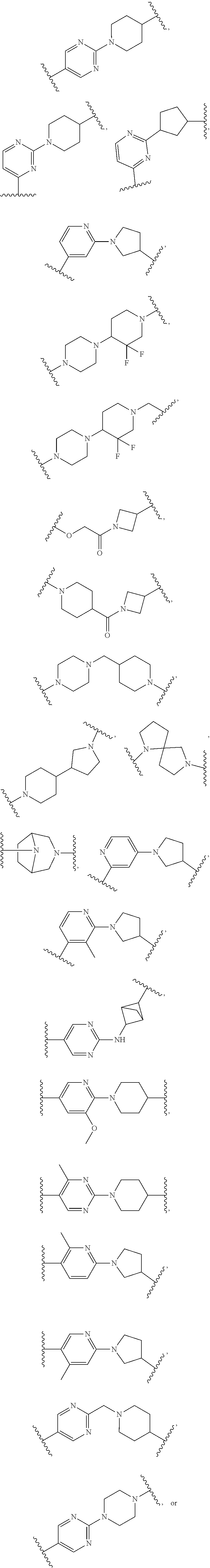

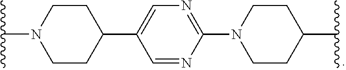 [SMILES: *C1CCN(c2ncc(C3CCN(*)CC3)cn2)CC1]

Embodiment 173. The compound of Embodiment 147, or a pharmaceutically acceptable salt thereof, wherein the linker has the structure of

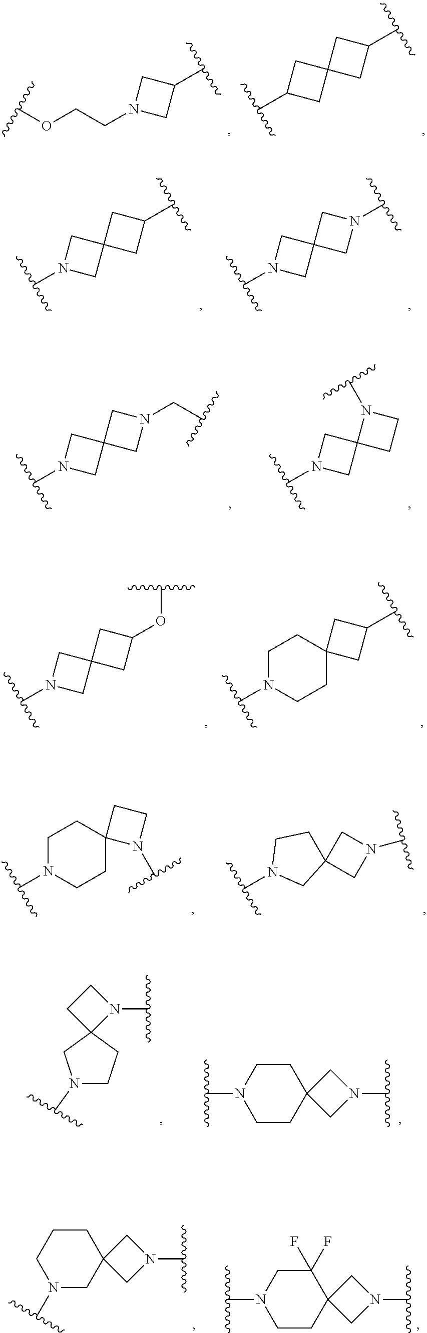

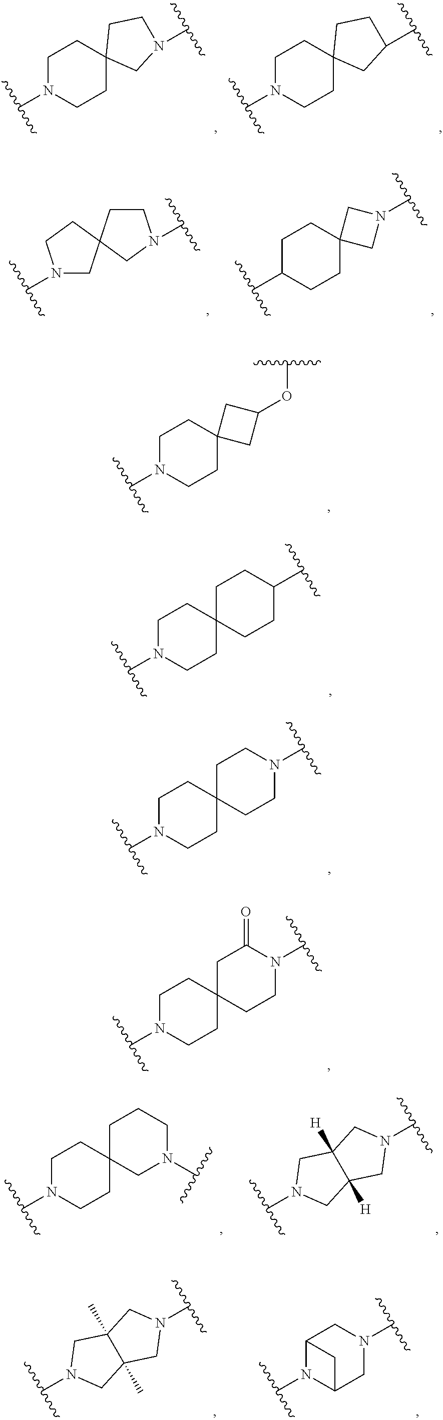

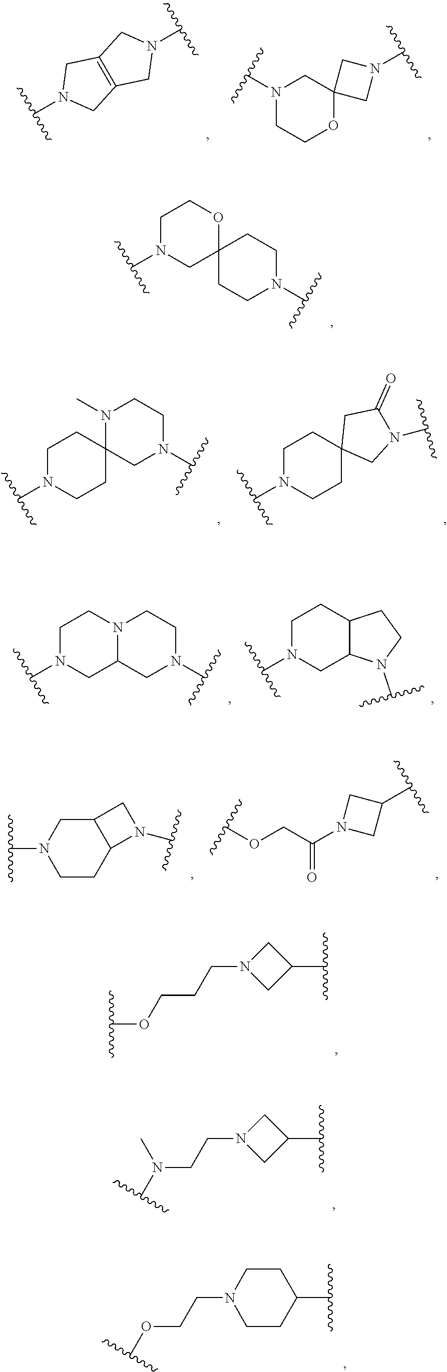

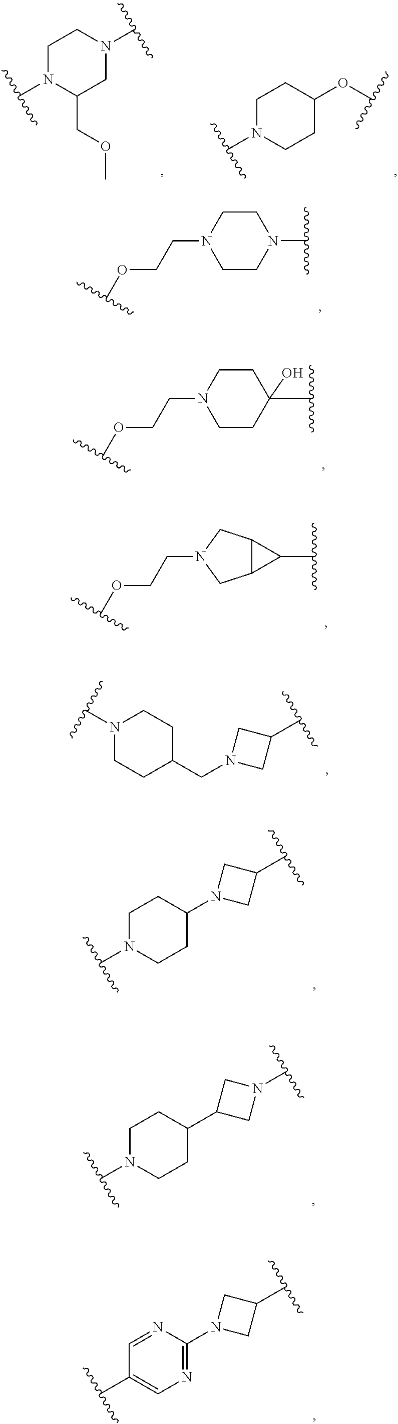

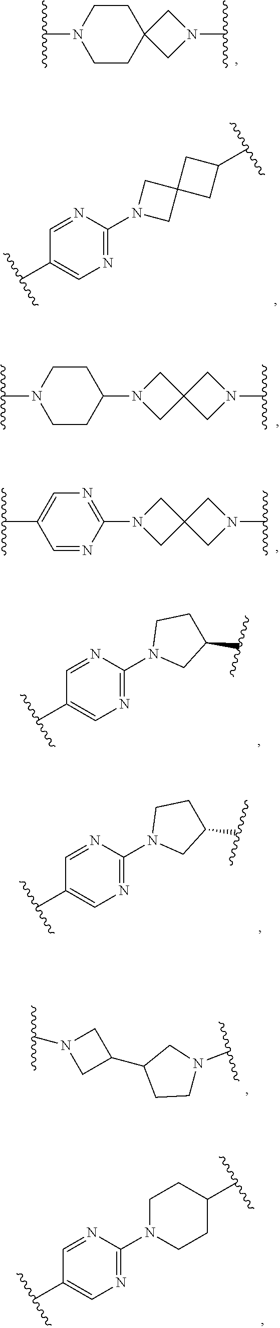

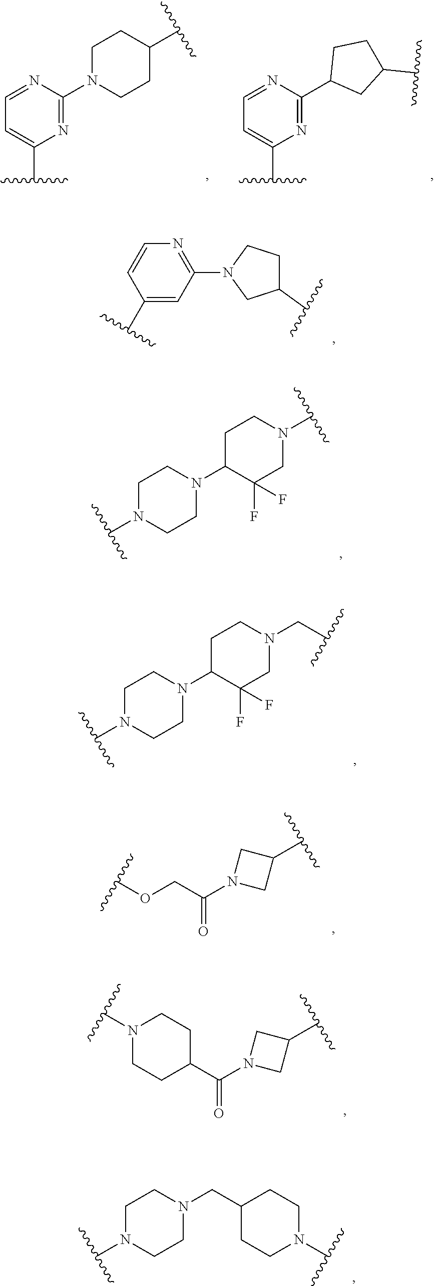

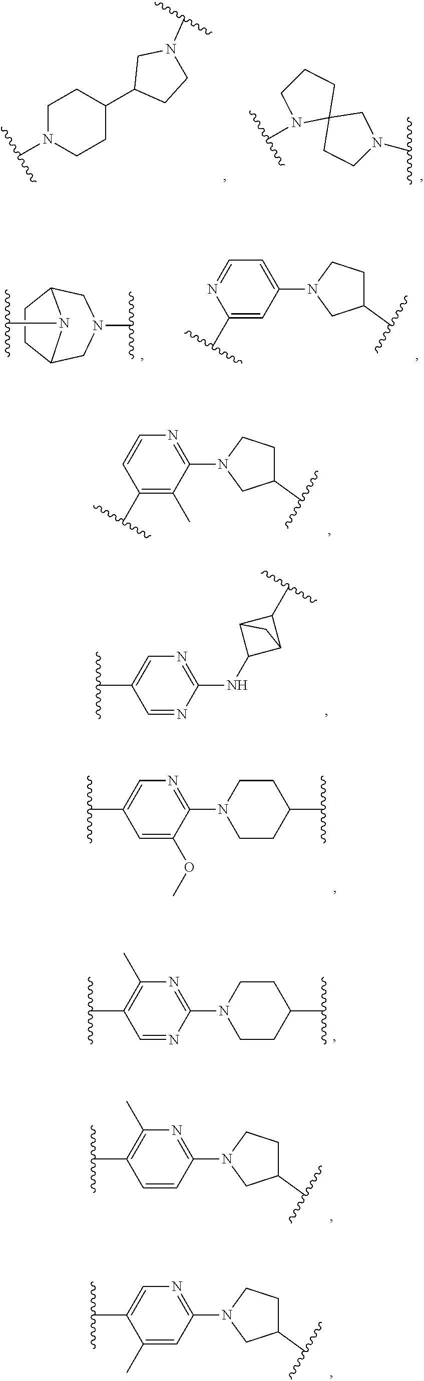

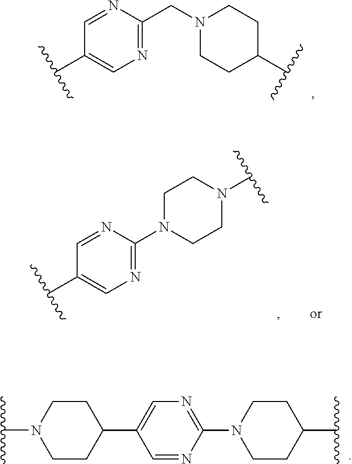 [SMILES: *c1cnc(CN2CCC(*)CC2)nc1 ; *c1cnc(N2CCN(*)CC2)nc1 ; *C1CCN(c2ncc(C3CCN(*)CC3)cn2)CC1]

Embodiment 174. The compound of any one of Embodiments 147-148, or a pharmaceutically acceptable salt thereof, wherein the linker has the structure of

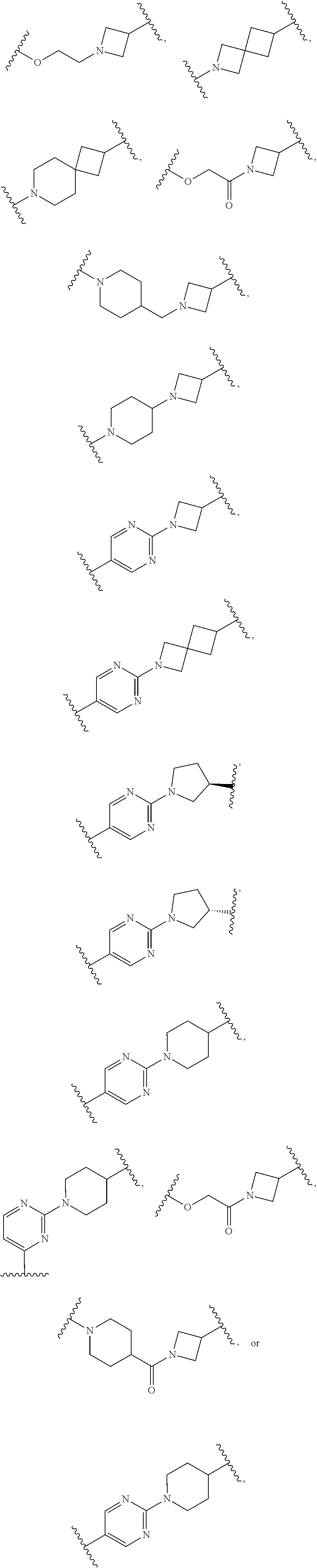

Embodiment 175. The compound of any one of Embodiments 1 to 146, or a pharmaceutically acceptable salt thereof, wherein the linker has the structure of Formula III:

A¹-(B¹)_{f}-(C¹)_{g}-(B²)ₕ-(B³)ᵢ-(C²)ⱼ-(B⁴)ₖ-A², Formula III

wherein
A¹ is a bond between the linker and ring system A;
A² is a bond between the degradation moiety and the linker;
each of B¹, B², B³, and B⁴ is, independently, optionally substituted ethynyl, optionally substituted C₆-C₁₀ aryl, optionally substituted C₃-C₁₀ cycloalkyl, optionally substituted C₃-C₁₀ carbocyclyl, optionally substituted C₂-C₁₀ heterocyclyl, optionally substituted C₂-C₉ heteroaryl, O, S, S(O)₂, or NRN;
each RN is, independently, H, optionally substituted C₁₋₄ alkyl, optionally substituted C₂₋₄ alkenyl, optionally substituted C₂₋₄ alkynyl, optionally substituted C₂₋₁₀ heterocyclyl, optionally substituted C₆₋₁₂ aryl, or optionally substituted C₁₋₇ heteroalkyl;
each of C¹ and C² is, independently, carbonyl, thiocarbonyl, sulphonyl, or phosphoryl; and
each of f, g, h, i, j, and k is, independently, 0 or 1.

Embodiment 176. The compound of Embodiment 175, or a pharmaceutically acceptable salt thereof, wherein the linker is of structure -(L¹)ₙ-, wherein n is 1, 2, or 3, and each L¹ is independently O, NRN, ethynyl, optionally substituted C₂-C₁₀ heterocyclyl, optionally substituted C₂-C₉ heteroaryl, optionally substituted C₆-C₁₀ aryl, optionally substituted C₃-C₁₀ cycloalkyl

Embodiment 177. The compound of Embodiment 176, or a pharmaceutically acceptable salt thereof, wherein at least one L¹ is optionally substituted C₂-C₁₀ heterocyclyl.

Embodiment 178. The compound of Embodiment 177, or a pharmaceutically acceptable salt thereof, wherein the optionally substituted C₂-C₁₀ heterocyclyl is 4-, 5-, or 6-membered monocyclic heterocyclyl, spirocyclic heterocyclyl, bridged heterocyclyl, or fused bicyclic heterocyclyl.

Embodiment 179. The compound of Embodiment 178, or a pharmaceutically acceptable salt thereof, wherein the C₂-C₁₀ heterocyclyl is:

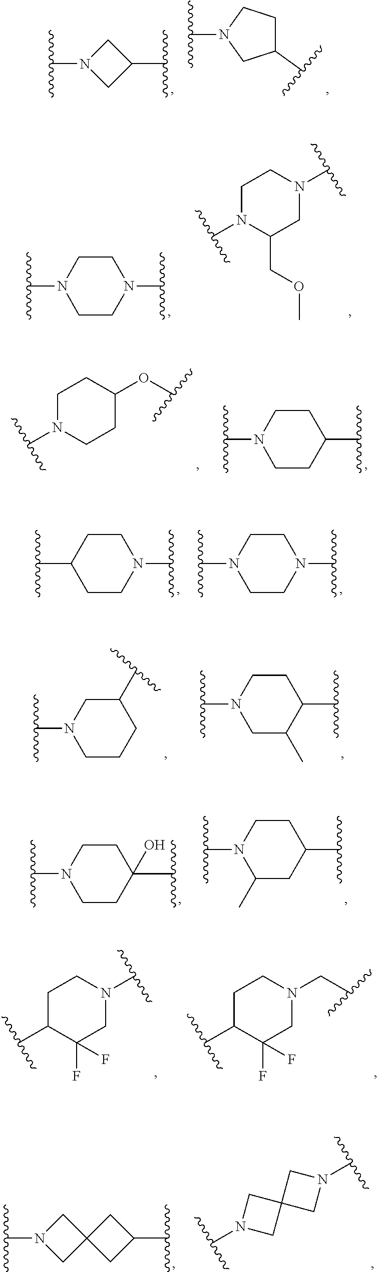

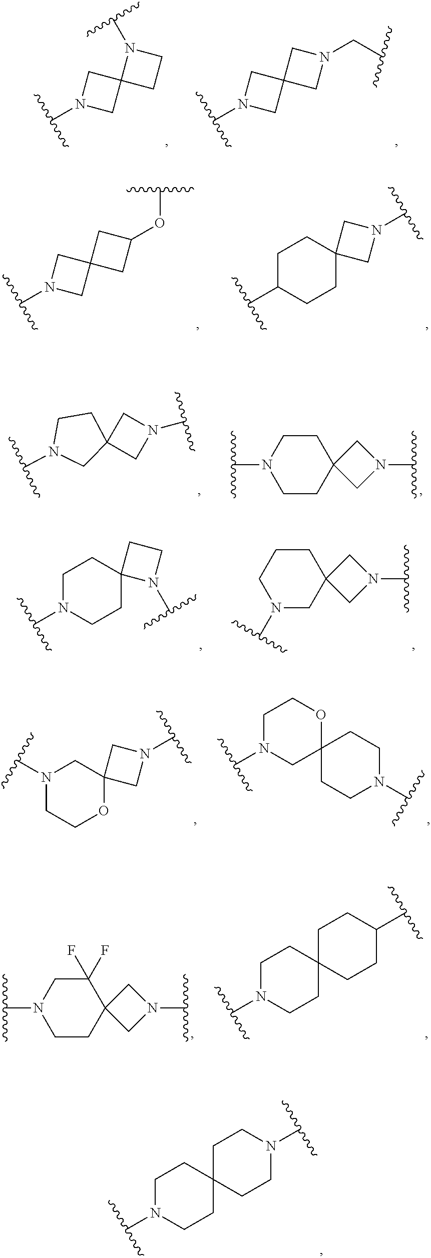

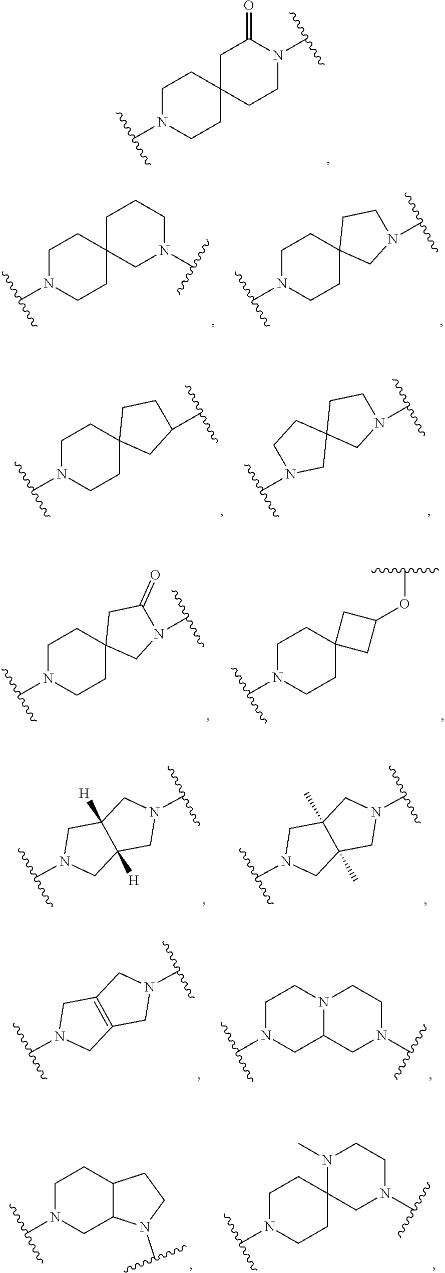

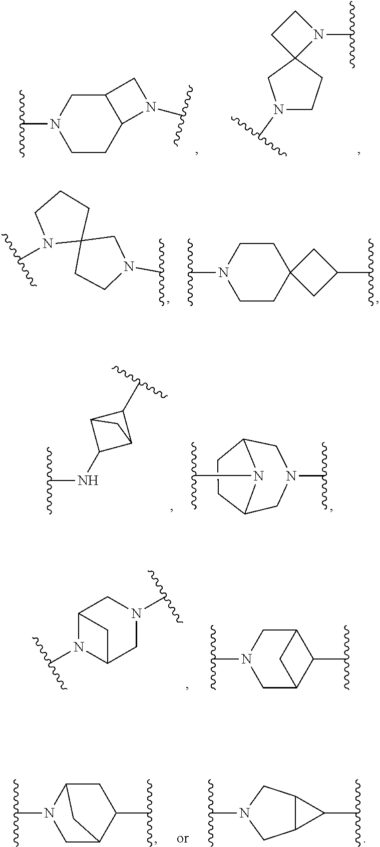

Embodiment 180. The compound of any one of Embodiments 176 to 179, or a pharmaceutically acceptable salt thereof, wherein at least one L¹ is optionally substituted C₂-C₉ heteroaryl.

Embodiment 181. The compound of any one of Embodiments 176 to 180, or a pharmaceutically acceptable salt thereof, wherein the linker is -(L¹)_{q}-(optionally substituted C₂-C₉ heteroaryl)-(L¹)_{q}-, wherein each q is independently 0 or 1.

Embodiment 182. The compound of Embodiment 180 or 181, or a pharmaceutically acceptable salt thereof, wherein the optionally substituted C₂-C₉ heteroaryl is a 6-membered monocyclic heteroaryl.

Embodiment 183. The compound of Embodiment 182, or a pharmaceutically acceptable salt thereof, wherein the 6-membered monocyclic heteroaryl is:

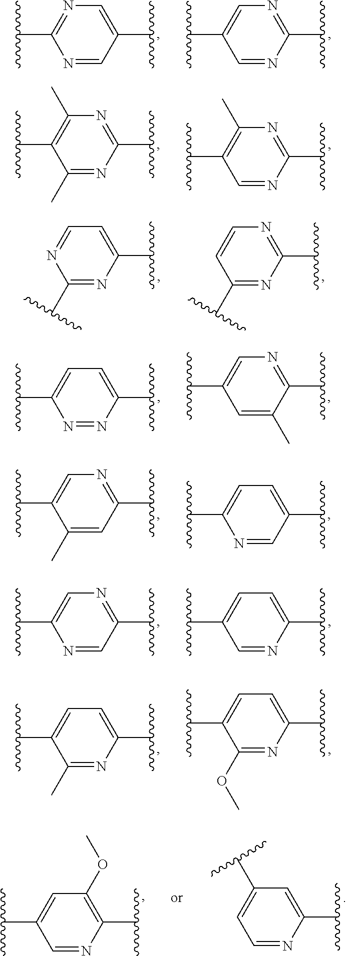

Embodiment 184. The compound of any one of Embodiments 176 to 183, or a pharmaceutically acceptable salt thereof, wherein at least one L¹ is optionally substituted C₆-C₁₀ aryl.

Embodiment 185. The compound of Embodiment 184, wherein the optionally substituted C₆-C₁₀ aryl is optionally substituted phenyl.

Embodiment 186. The compound of any one of Embodiments 176 to 185, or a pharmaceutically acceptable salt thereof, wherein at least one L¹ is optionally substituted C₃-C₁₀ cycloalkyl.

Embodiment 187. The compound of Embodiment 186, or a pharmaceutically acceptable salt thereof, wherein the optionally substituted C₃-C₁₀ cycloalkyl:

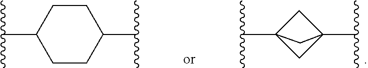 [SMILES: *C1CCC(*)CC1 ; *C12CC(*)(C1)C2]

Embodiment 188. The compound of any one of Embodiments 176 to 187, or a pharmaceutically acceptable salt thereof, wherein at least one L¹ is ethynyl.

Embodiment 189. The compound of any one of Embodiments 176 to 188, or a pharmaceutically acceptable salt thereof, wherein one and only one L¹ is O.

Embodiment 190. The compound of any one of Embodiments 176 to 188, or a pharmaceutically acceptable salt thereof, wherein one and only one L¹ is NR^{N}.

Embodiment 191. The compound of Embodiment 190, or a pharmaceutically acceptable salt thereof, wherein R^{N} is H or optionally substituted C₁-C₄ alkyl.

Embodiment 192. The compound of Embodiment 175, or a pharmaceutically acceptable salt thereof, wherein the linker is of the following structure:

A¹-(B¹)_{f}-(B²)ₕ-(B³)ᵢ-(B⁴)ₖ-A²,

wherein each of B¹, B², B³, and B⁴ is, independently, optionally substituted ethynyl, optionally substituted C₆-C₁₀ aryl, optionally substituted C₃-C₁₀ cycloalkyl, optionally substituted C₂-C₁₀ heterocyclyl, optionally substituted C₂-C₉ heteroaryl, O, or NR^{N}.

Embodiment 193. The compound of Embodiment 175 or 192, or a pharmaceutically acceptable salt thereof, wherein at least one of f, h, i, and k is 1.

Embodiment 194. The compound of any one of Embodiments 175 or 192 to 193, or a pharmaceutically acceptable salt thereof, wherein each of B¹, B², B³, and B⁴ is, independently, O, ethynyl, optionally substituted C₂-C₉ heteroaryl, optionally substituted C₂-C₁₀ heterocyclyl, optionally substituted C₃-C₁₀ cycloalkyl, or optionally substituted C₆-C₁₀ aryl.

Embodiment 195. The compound of any one of Embodiments 175 and 192 to 194, or a pharmaceutically acceptable salt thereof, wherein each of B¹, B², B³, and B⁴ is, independently optionally substituted C₂-C₉ heteroaryl or optionally substituted C₂-C₁₀ heterocyclyl.

Embodiment 196. The compound of any one of Embodiments 175 and 192 to 195, or a pharmaceutically acceptable salt thereof, wherein each of B¹ and B⁴ is, independently,

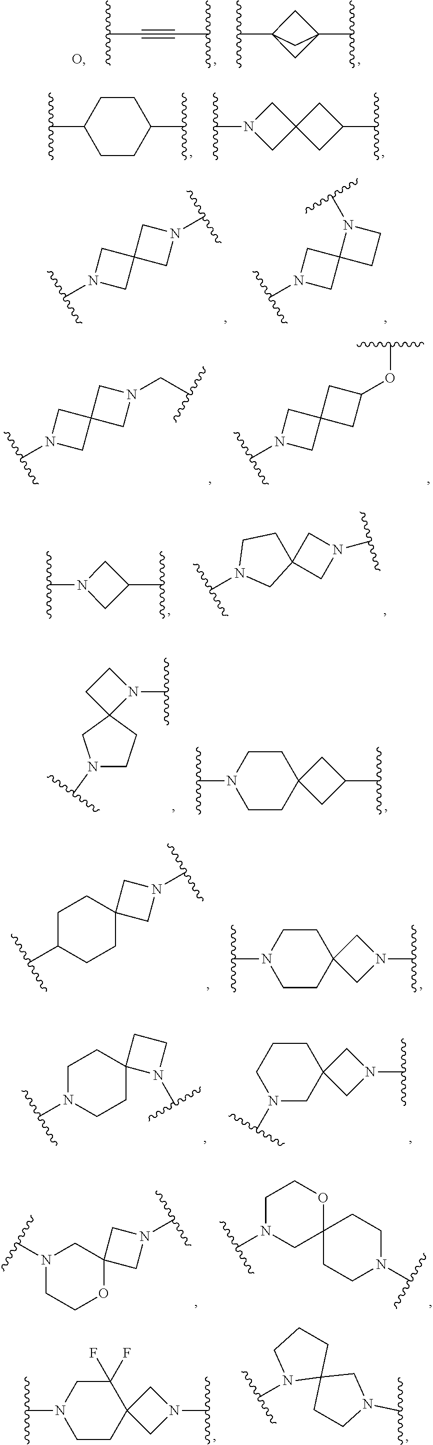

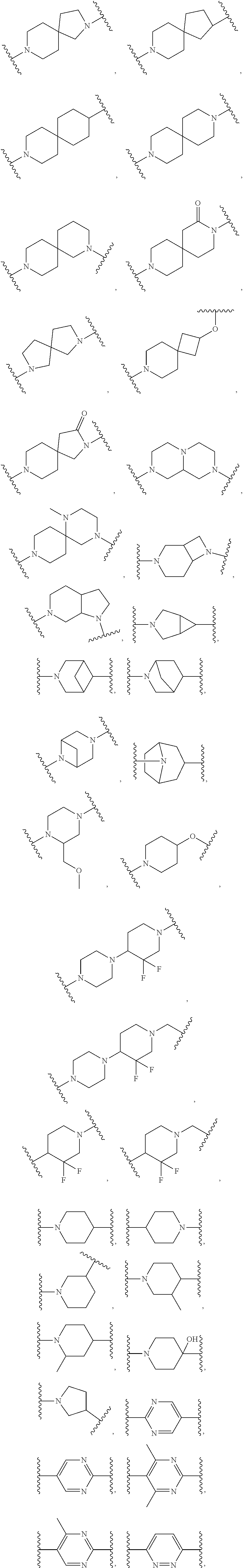

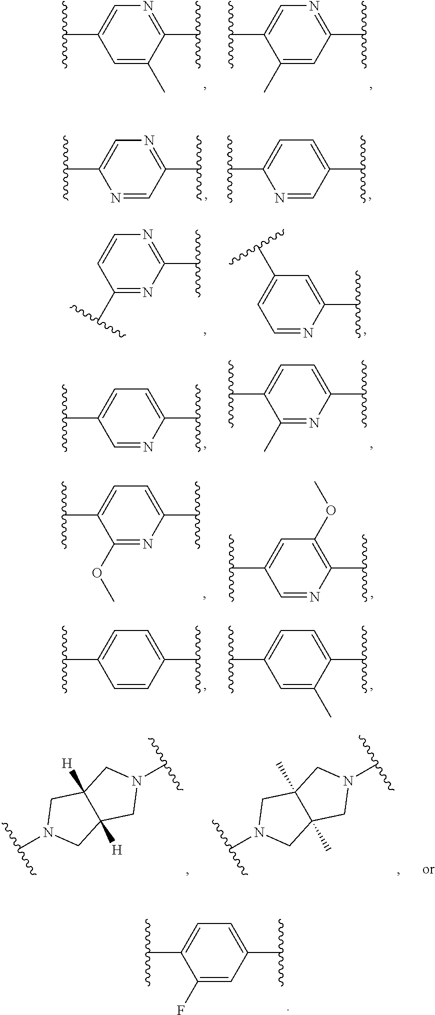

Embodiment 197. The compound of Embodiment 196, or a pharmaceutically acceptable salt thereof, wherein B¹ is

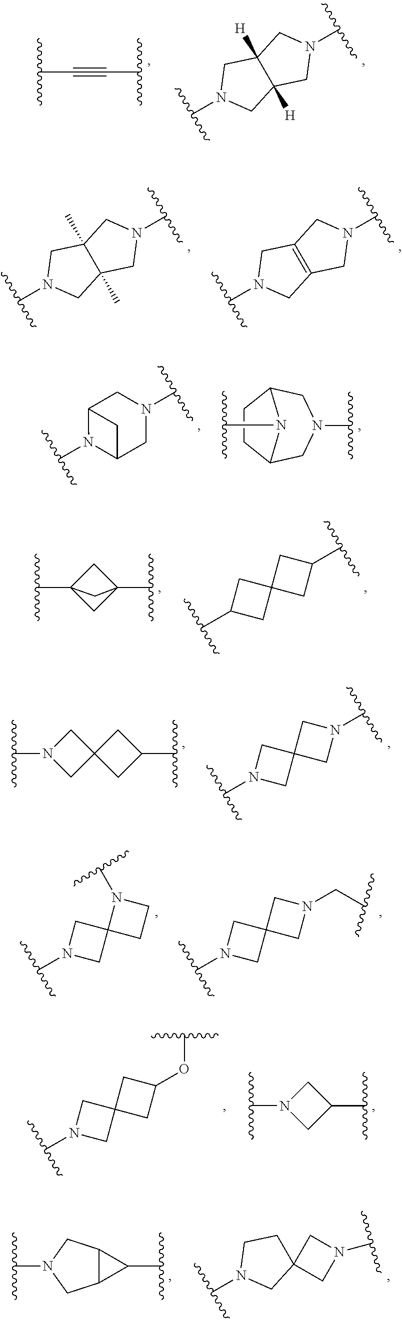

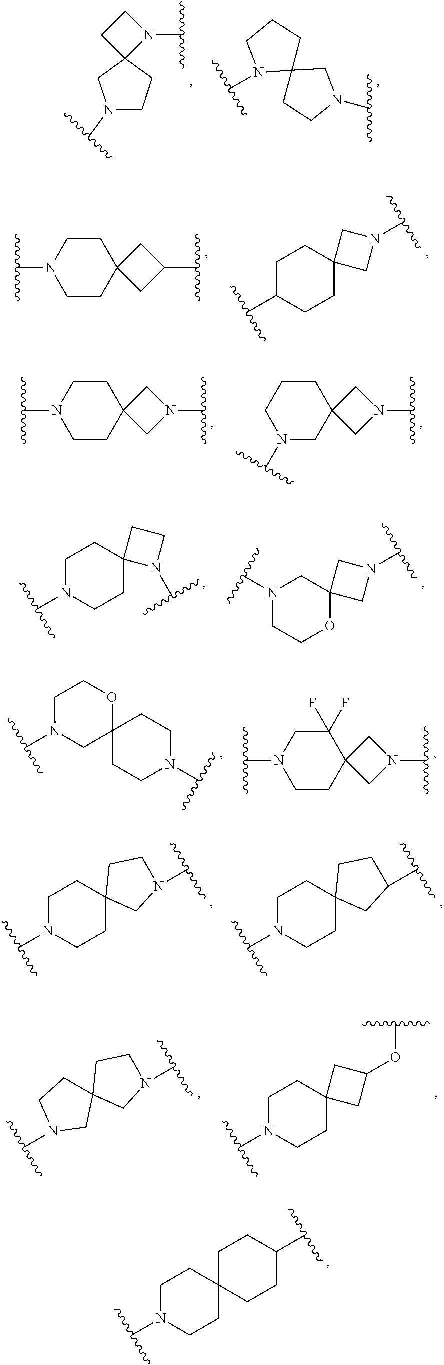

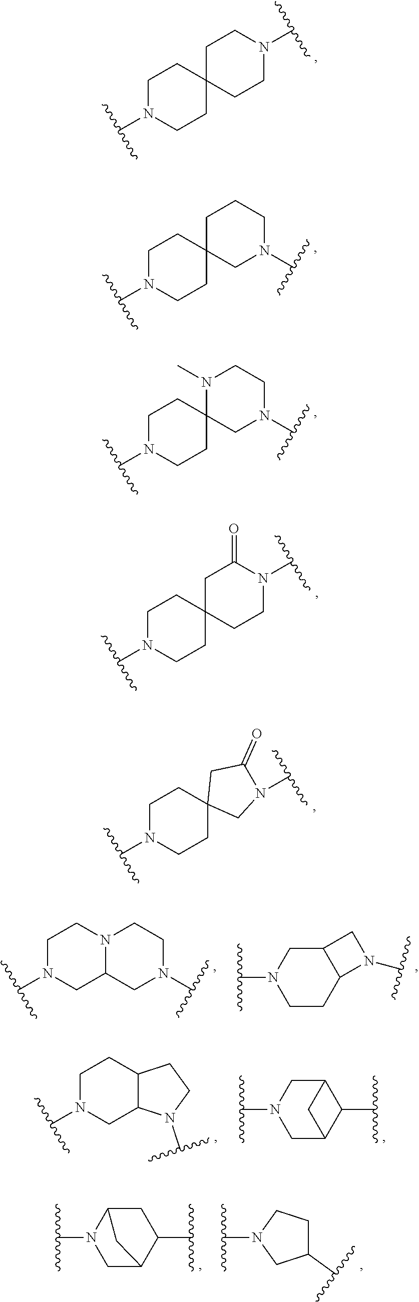

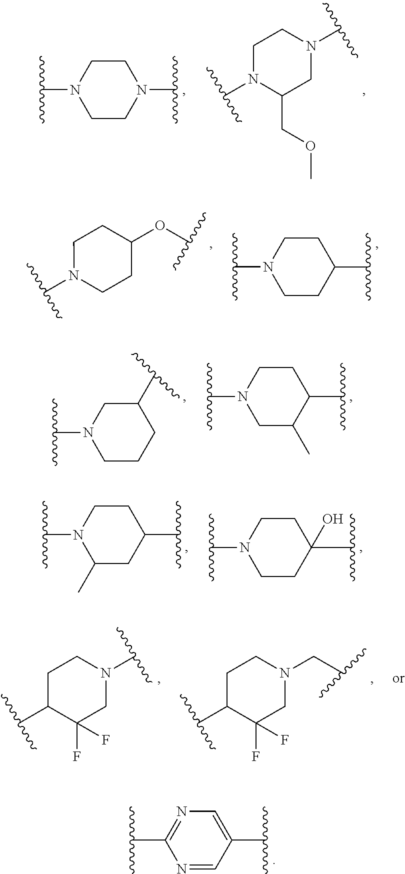

Embodiment 198. The compound of Embodiment 196 or 197, or a pharmaceutically acceptable salt thereof, wherein B⁴ is

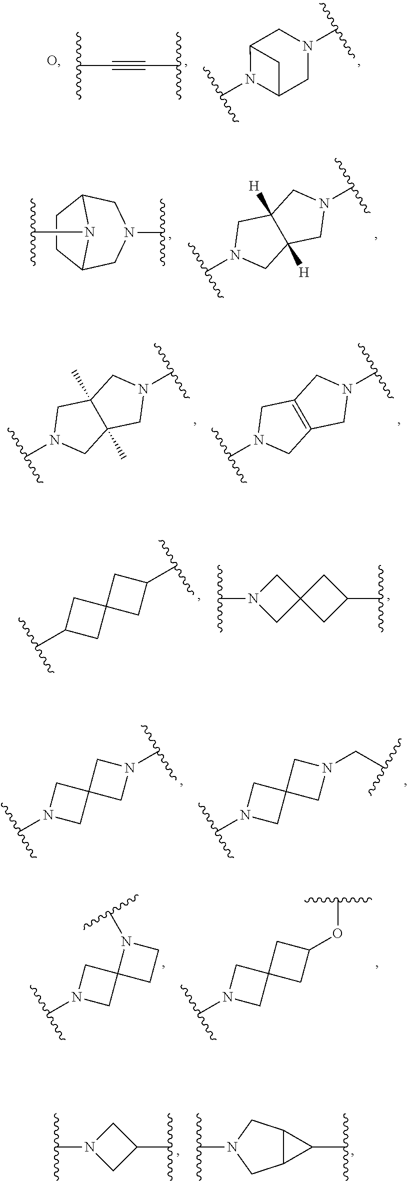

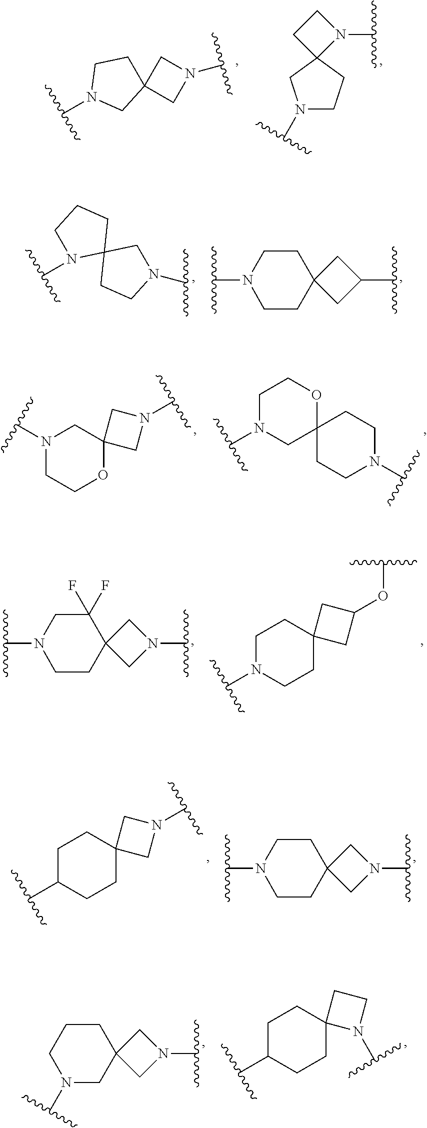

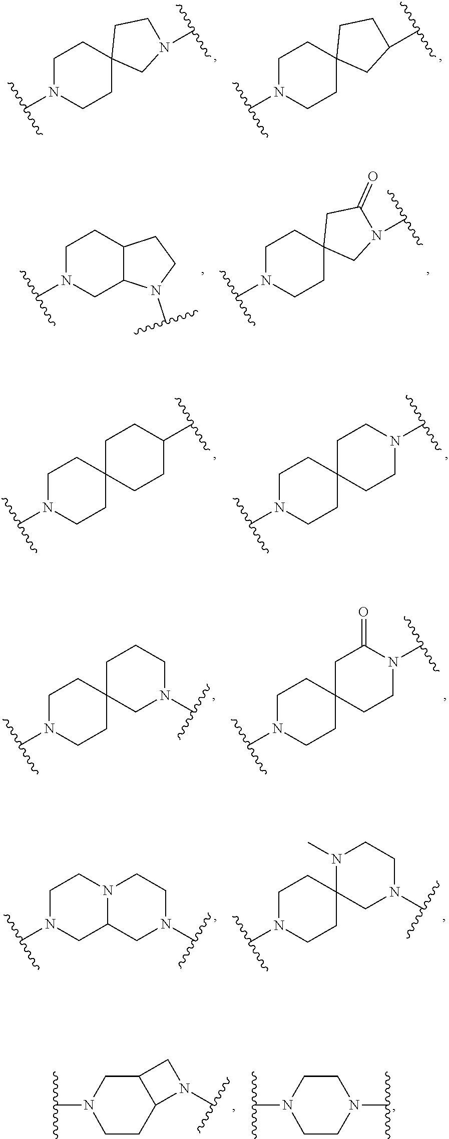

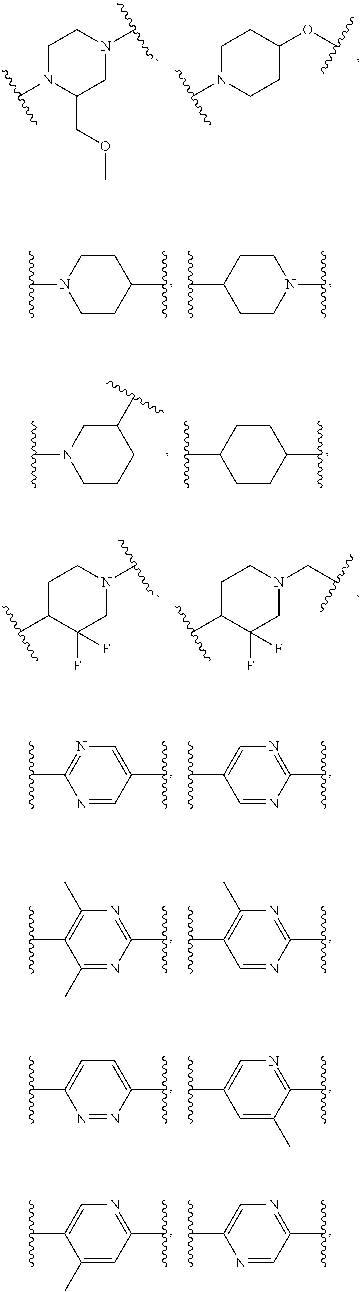

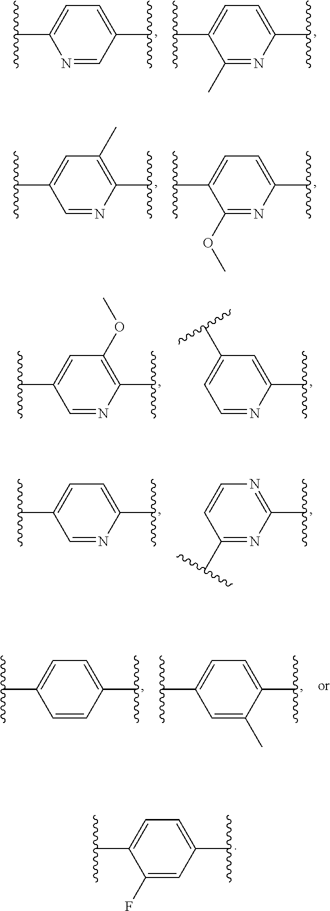

Embodiment 199. The compound of any one of Embodiments 175 and 192 to 198, or a pharmaceutically acceptable salt thereof, wherein B² is NH

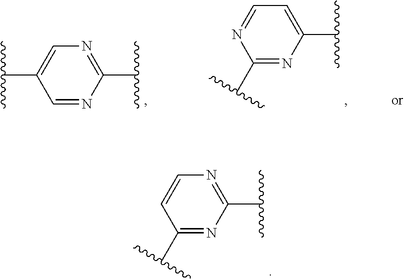 [SMILES: *c1cnc(*)nc1 ; *c1ccnc(*)n1 ; *c1ccnc(*)n1]

Embodiment 200. The compound of any one of Embodiments 175 and 192 to 199, or a pharmaceutically acceptable salt thereof, wherein f is 0.

Embodiment 201. The compound of any one of Embodiments 175 and 191 to 199, or a pharmaceutically acceptable salt thereof, wherein f is 1.

Embodiment 202. The compound of any one of Embodiments 175 and 192 to 201, or a pharmaceutically acceptable salt thereof, wherein g, h, l and j are 0.

Embodiment 203. The compound of any one of Embodiments 175 and 192 to 202, or a pharmaceutically acceptable salt thereof, wherein k is 0.

Embodiment 204. The compound of any one of Embodiments 175 and 192 to 202, or a pharmaceutically acceptable salt thereof, wherein k is 1.

Embodiment 205. The compound of Embodiment 175, or a pharmaceutically acceptable salt thereof, wherein the linker has the structure of

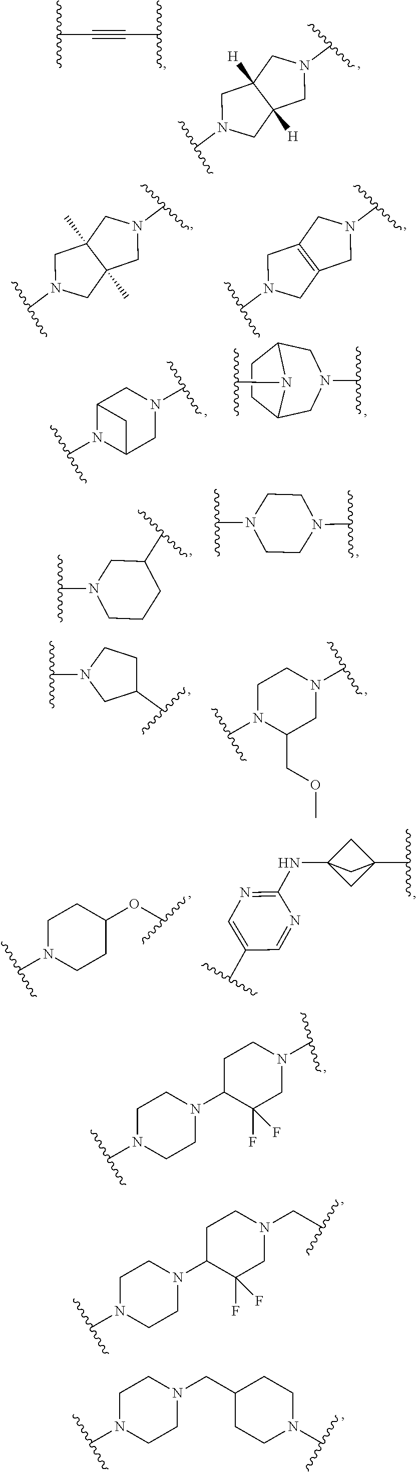

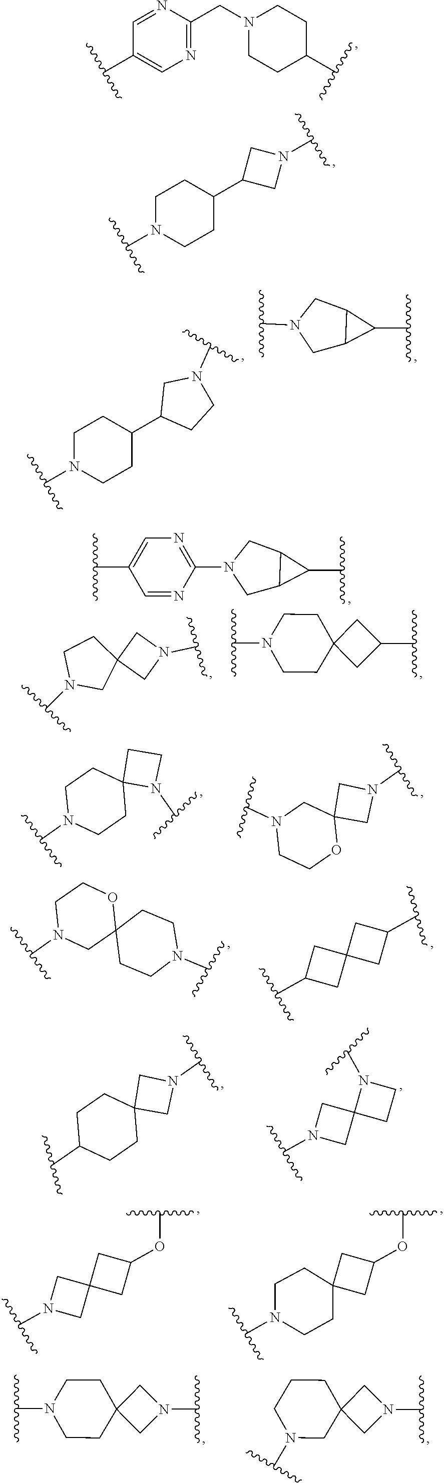

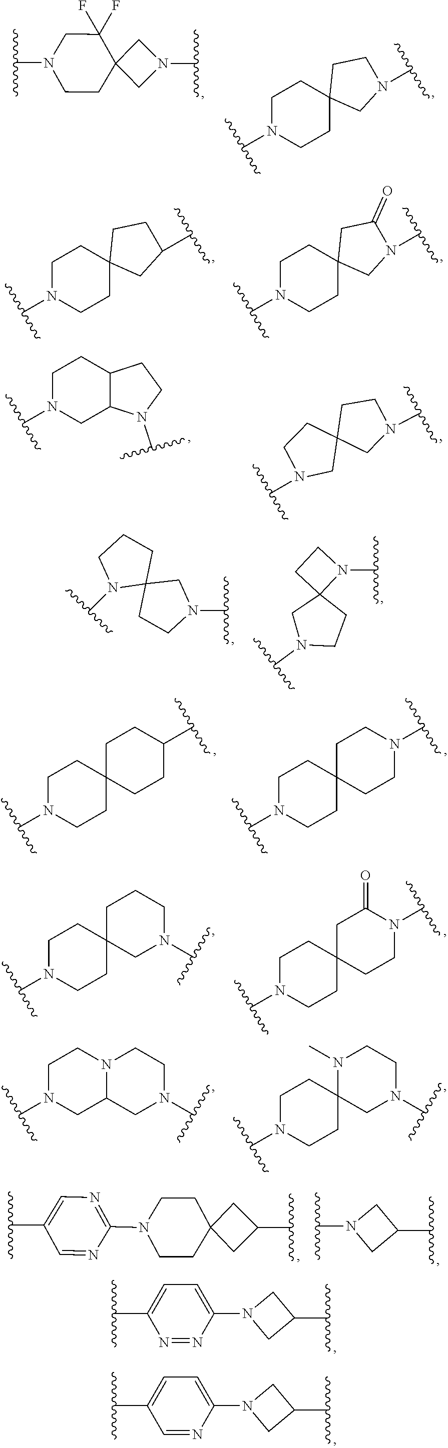

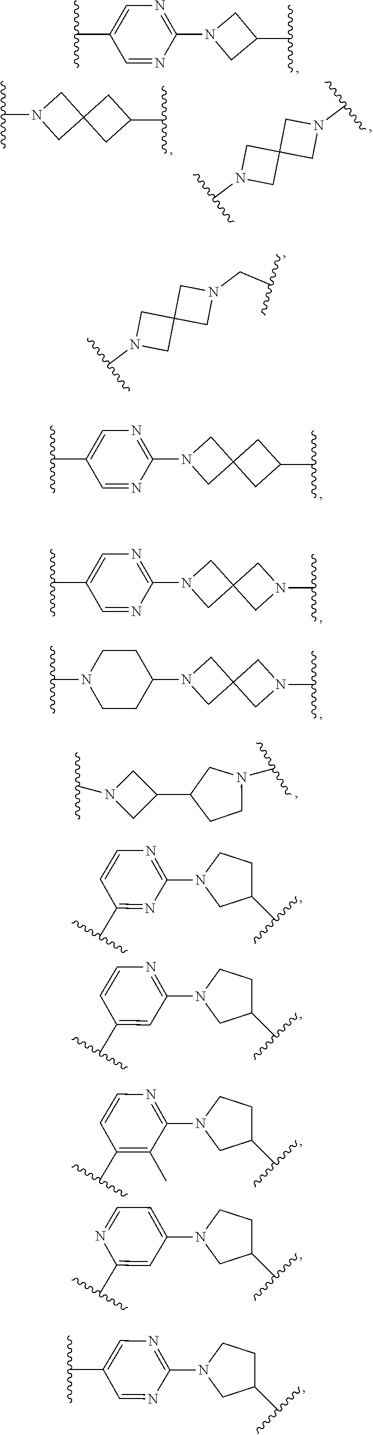

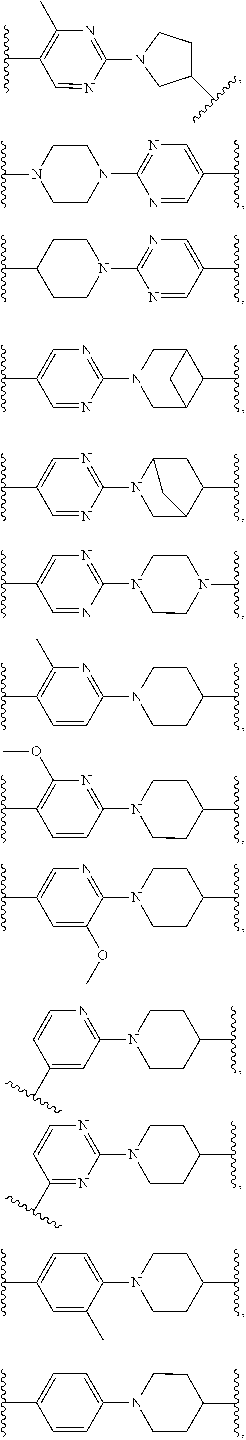

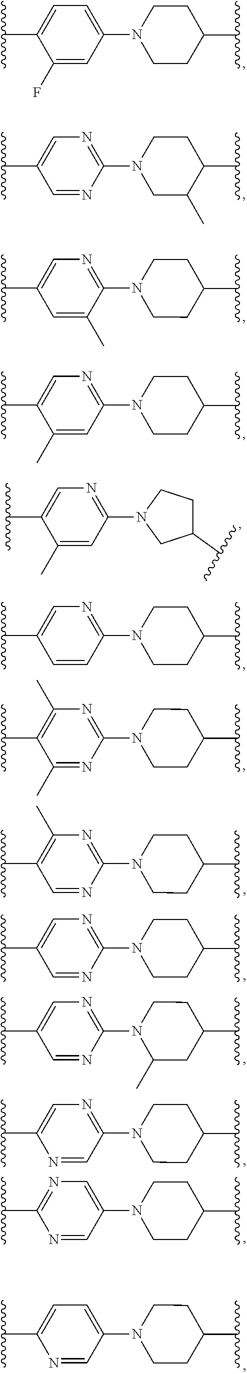

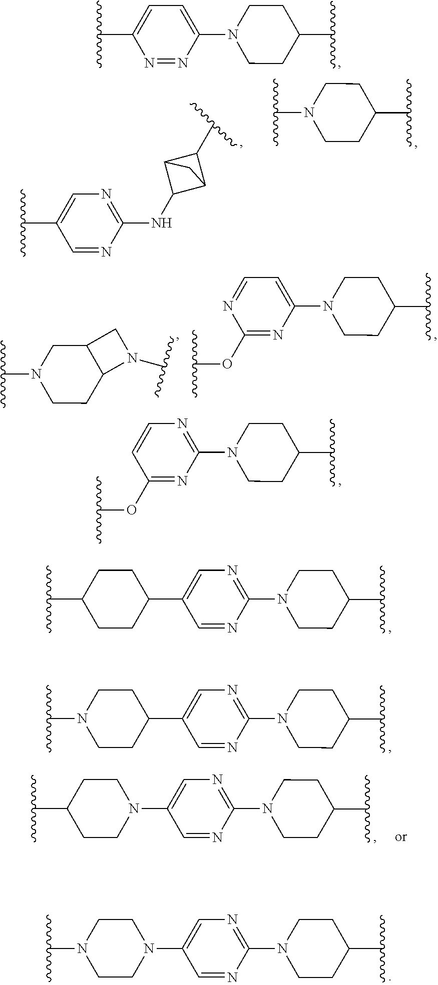

Embodiment 206. A compound selected from the group consisting of compounds 1-291 in Table 1 and pharmaceutically acceptable salts thereof.

Embodiment 207. The compound of any one of Embodiments 1 to 206, or a pharmaceutically acceptable salt thereof, wherein the compound has a ratio of BRG1 IC₅₀ to BRM IC₅₀ of at least 5.

Embodiment 208. The compound of any one of Embodiments 1 to 206, or a pharmaceutically acceptable salt thereof, wherein the compound has a ratio of BRG1 IC₅₀ to BRM IC₅₀ of at least 10.

Embodiment 209. The compound of any one of Embodiments 1 to 206, or a pharmaceutically acceptable salt thereof, wherein the compound has a ratio of BRG1 IC₅₀ to BRM IC₅₀ of at least 20.

Embodiment 210. The compound of any one of Embodiments 1 to 206, or a pharmaceutically acceptable salt thereof, wherein the compound has a ratio of BRG1 IC₅₀ to BRM IC₅₀ of at least 30.

Embodiment 211. A pharmaceutical composition comprising a compound of any one of Embodiments 1 to 210 and a pharmaceutically acceptable excipient.

Embodiment 212. A method of treating a BAF complex-related disorder in a subject in need thereof, the method comprising administering to the subject an effective amount of a compound of any one of Embodiments 1 to 210 or a pharmaceutical composition of Embodiment 211.

Embodiment 213. The method of Embodiment 212, wherein the BAF complex-related disorder is cancer or a viral infection.

Embodiment 214. A method of treating a disorder related to a BRG1 loss of function mutation in a subject in need thereof, the method comprising administering to the subject an effective amount of a compound of any one of Embodiments 1 to 210 or a pharmaceutical composition of Embodiment 211.

Embodiment 215. The method of Embodiment 214, wherein the disorder related to a BRG1 loss of function mutation is cancer.

Embodiment 216. A method of treating cancer in a subject in need thereof, the method comprising administering to the subject an effective amount of a compound of any one of Embodiments 1 to 210 or a pharmaceutical composition of Embodiment 211.

Embodiment 217. The method of any one of Embodiments 212-216, wherein the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

Embodiment 218. The method of Embodiment 217, wherein the cancer is non-small cell lung cancer, colorectal cancer, bladder cancer, cancer of unknown primary, glioma, breast cancer, melanoma, non-melanoma skin cancer, endometrial cancer, or penile cancer.

Embodiment 219. The method of Embodiment 217, wherein the cancer is non-small cell lung cancer.

Embodiment 220. The method of Embodiment 217, wherein the cancer is soft tissue sarcoma.

Embodiment 221. A method of treating a cancer selected from the group consisting of melanoma, prostate cancer, breast cancer, bone cancer, renal cell carcinoma, and a hematologic cancer in a subject in need thereof, the method comprising administering to the subject an effective amount of a compound of any one of Embodiments 1 to 210 or a pharmaceutical composition of Embodiment 211.

Embodiment 222. A compound of any one of Embodiments 1 to 210, or a pharmaceutically acceptable salt thereof, or a pharmaceutical composition of Embodiment 211, for use in therapy.

Embodiment 223. A compound of any one of Embodiments 1 to 210, or a pharmaceutically acceptable salt thereof, or a pharmaceutical composition of Embodiment 211, for use in treating cancer.

Embodiment 224. The compound, or the pharmaceutically acceptable salt thereof, or the pharmaceutical composition for use according to Embodiment 223, wherein the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

Embodiment 225. The compound, or the pharmaceutically acceptable salt thereof, or the pharmaceutical composition for use according to Embodiment 223, wherein the cancer is non-small cell lung cancer, colorectal cancer, bladder cancer, cancer of unknown primary, glioma, breast cancer, melanoma, non-melanoma skin cancer, endometrial cancer, or penile cancer.

Embodiment 226. The compound, or the pharmaceutically acceptable salt thereof, or the pharmaceutical composition for use according to Embodiment 223, wherein the cancer is non-small cell lung cancer.

Embodiment 227. The compound, or the pharmaceutically acceptable salt thereof, or the pharmaceutical composition for use according to Embodiment 223, wherein the cancer is soft tissue sarcoma.

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1. A compound, or a pharmaceutically acceptable salt thereof, of Formula I: 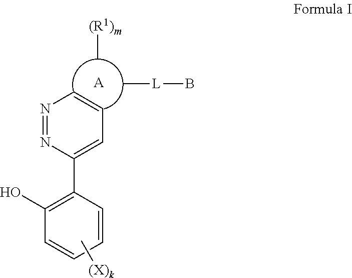
wherein
ring system A is a 5 to 9-membered heterocyclyl or heteroaryl containing at least one N;
m is 0, 1, 2, or 3;
k is 0, 1, or 2;
each R¹ is, independently, halo, optionally substituted C₁-C₆ alkyl, optionally substituted C₁-C₆ heteroalkyl, optionally substituted C₂-C₉ heterocyclyl, optionally substituted C₃-C₈ cycloalkyl, or optionally substituted CH₂—C₃-C₈ cycloalkyl;
each X is, independently, halo;
L is a linker, wherein the linker has the structure of Formula II:
A¹-(B¹)_{f}-(C¹)_{g}-(B²)ₕ-(D)-(B³)ᵢ-(C²)ⱼ-(B⁴)ₖ-A², Formula II
or a pharmaceutically acceptable salt thereof,
wherein
A¹ is a bond between the linker and the ring system A;
A² is a bond between the degradation moiety and the linker;
each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₃-C₁₀ carbocyclyl, optionally substituted C₂-C₁₀ heterocyclyl, optionally substituted C₂-C₆ heteroaryl, optionally substituted C₆₋₁₂ aryl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₁₀ heterocyclyl, optionally substituted C₂₋₆ heteroaryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₁₀ heterocyclyl, optionally substituted C₂₋₆ heteroaryl, optionally substituted C₆₋₁₂ aryl, optionally substituted C₂-C₁₀ polyethylene glycol, optionally substituted C₃-C₁₀ cycloalkyl, optionally substituted C₃-C₁₀ carbocyclyl, or optionally substituted C₁₋₁₀ heteroalkyl; or D is absent, and the linker is A¹-(B¹)_{f}-(C¹)_{g}-(B²)ₕ—(B³)ᵢ-(C²)ⱼ-(B⁴)ₖ-A²; and
B is a degradation moiety, wherein the degradation moiety has the structure of Formula C: 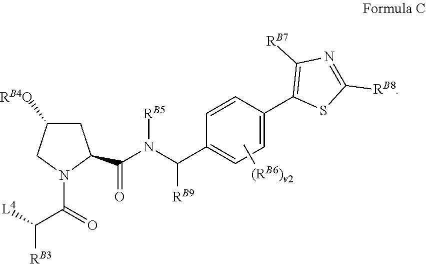
wherein
L⁴ is —N(R^{B1})(R^{B2}), 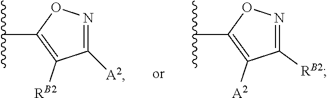
R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl; and
A² is a bond between the degradation moiety and the linker;
wherein one and only one of R^{B1}, R^{B3}, and R^{B6} is A²,
or a pharmaceutically acceptable salt thereof.

2. (canceled)

3. (canceled)

4. The compound of claim 1, or a pharmaceutically acceptable salt thereof, wherein the compound has the structure of Formula I-C: 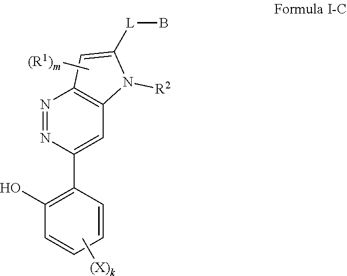
wherein R² is H, optionally substituted C₁-C₆ alkyl, optionally substituted C₃-C₈ cycloalkyl, optionally substituted C₂-C₉ heterocyclyl, or a bond to -L-B.

5.-8. (canceled)

9. The compound of claim 4, or a pharmaceutically acceptable salt thereof, R² is H, CH₃,

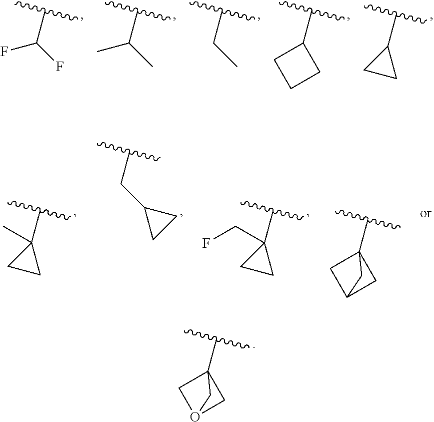

10. The compound of claim 9, or a pharmaceutically acceptable salt thereof, wherein R² is H, CH₃,

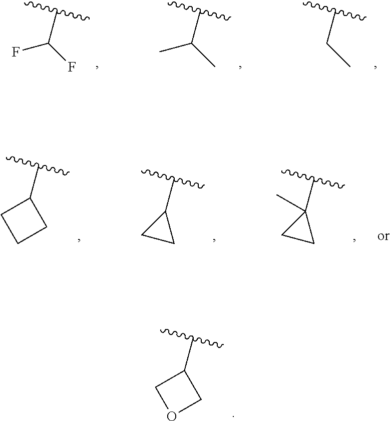

11. (canceled)

12. The compound of claim 1, or a pharmaceutically acceptable salt thereof, wherein m is 1.

13. (canceled)

14. (canceled)

15. The compound of claim 1 or a pharmaceutically acceptable salt thereof, wherein R¹ is methyl or difluoromethyl.

16. (canceled)

17. The compound of claim 1, or a pharmaceutically acceptable salt thereof, wherein k is 0.

18. (canceled)

19. (canceled)

20. The compound of claim 1, or a pharmaceutically acceptable salt thereof, wherein X is Cl or F.

21. The compound of claim 1, or a pharmaceutically acceptable salt thereof, wherein the compound has the structure of formula I-E, formula I-F, formula I-G, formula I-H or formula I-I:

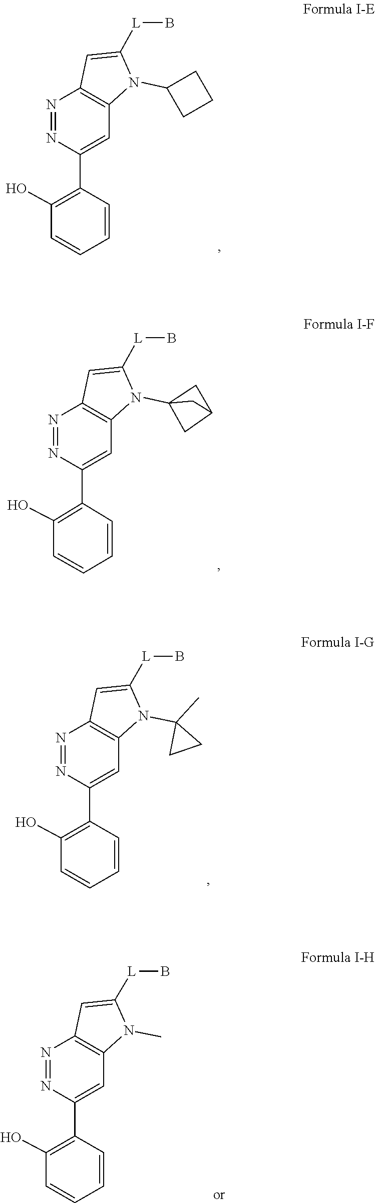

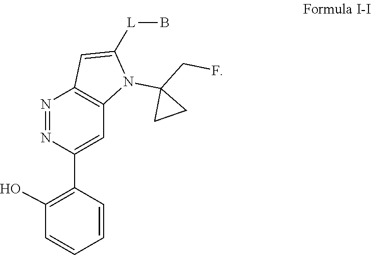

22. The compound of claim 1, or a pharmaceutically acceptable salt thereof, wherein the compound has the structure of formula I-J, formula I-K, formula I-L, formula I-M or formula I-N:

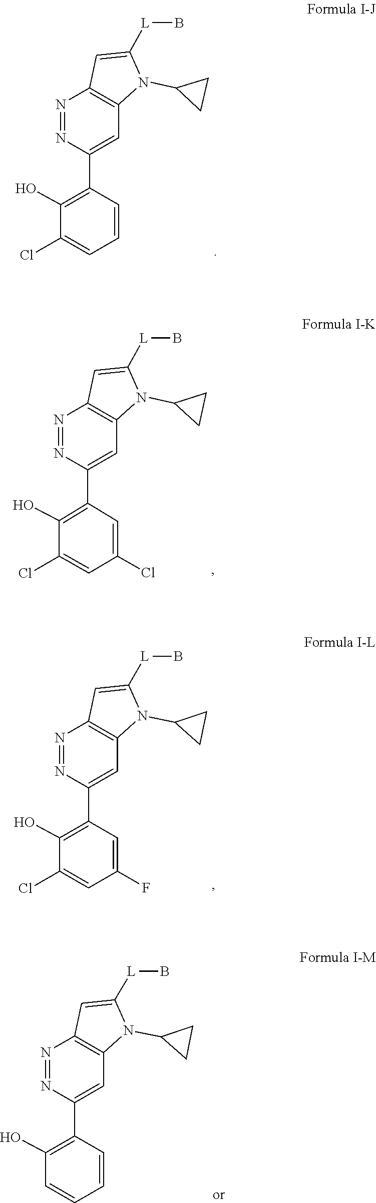

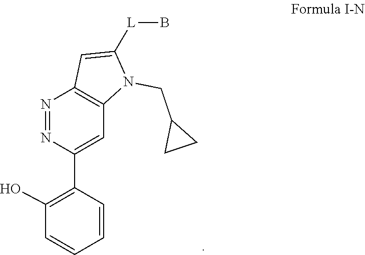

23. The compound of claim 1, or a pharmaceutically acceptable salt thereof, wherein the compound has the structure of formula I-O, formula I-P, formula I-Q, formula I-R or formula I-S:

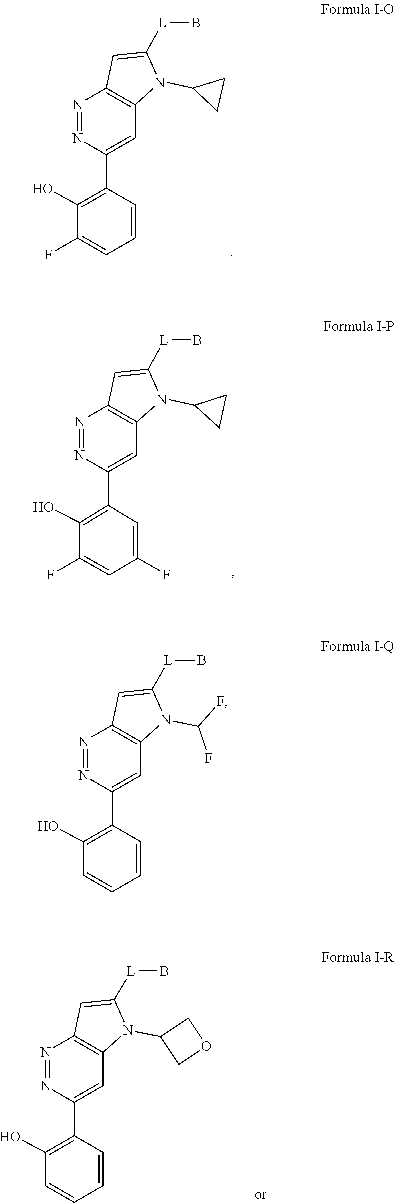

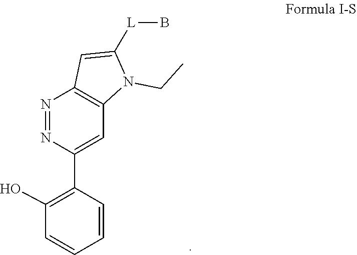

24. The compound of claim 1, or a pharmaceutically acceptable salt thereof, wherein the compound has the structure of formula I-T, formula I-U, formula I-V or formula I-W:

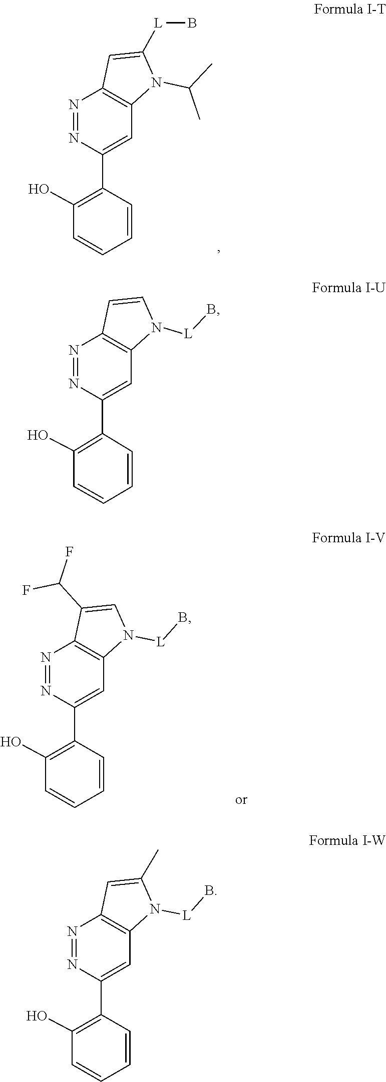

25.-30. (canceled)

31. The compound of claim 1, or a pharmaceutically acceptable salt thereof, wherein the degradation moiety is

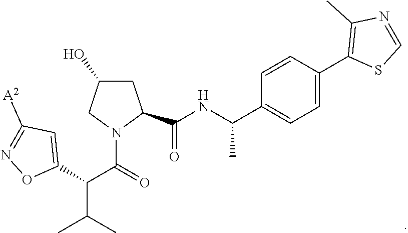

32.-47. (canceled)

48. The compound of claim 1, or a pharmaceutically acceptable salt thereof, wherein
A¹ is a bond between the linker and the benzopyridazine core ring system;
A² is a bond between the degradation moiety and the linker;
each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₂-C₈ heterocyclyl, optionally substituted C₂-C₆ heteroaryl, optionally substituted C₆₋₁₂ aryl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₂₋₆ heteroaryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₂₋₆ heteroaryl, optionally substituted C₆₋₁₂ aryl, optionally substituted C₂-C₁₀ polyethylene glycol, or optionally substituted C₁₋₁₀ heteroalkyl; or D is absent, and the linker is A¹-(B¹)_{f}—(C¹)_{g}-(B²)ₕ-(B³)ᵢ-(C²)ⱼ-(B⁴)ₖ-A².

49. The compound of claim 1, or a pharmaceutically acceptable salt thereof, wherein the linker has the structure of

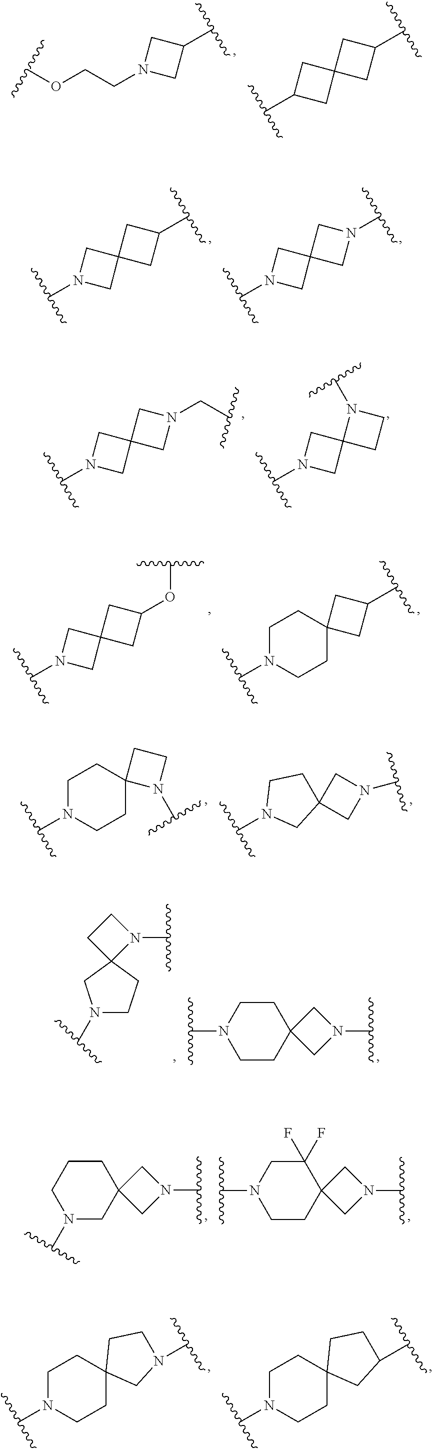

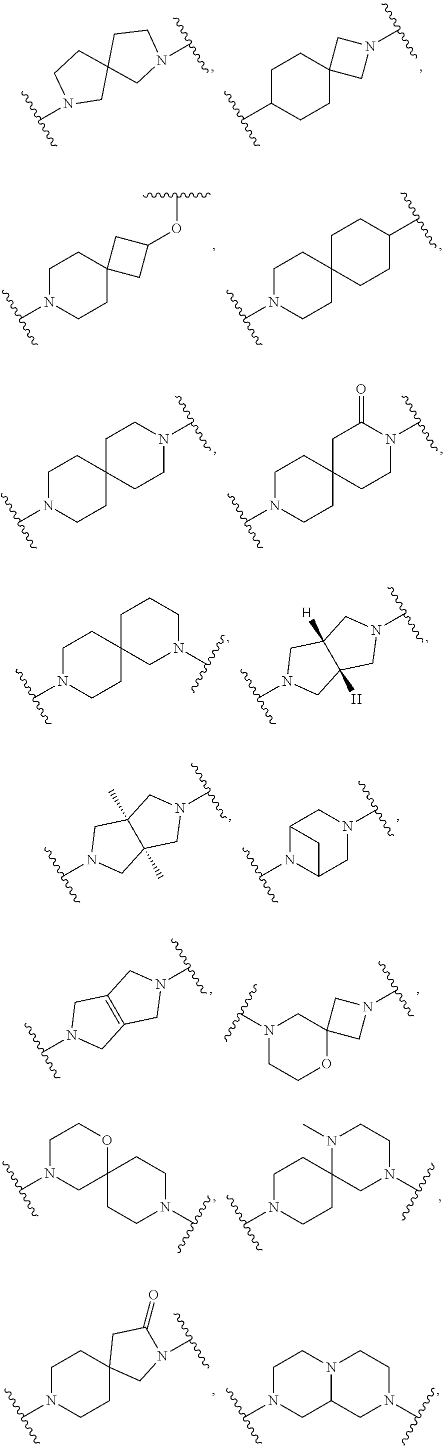

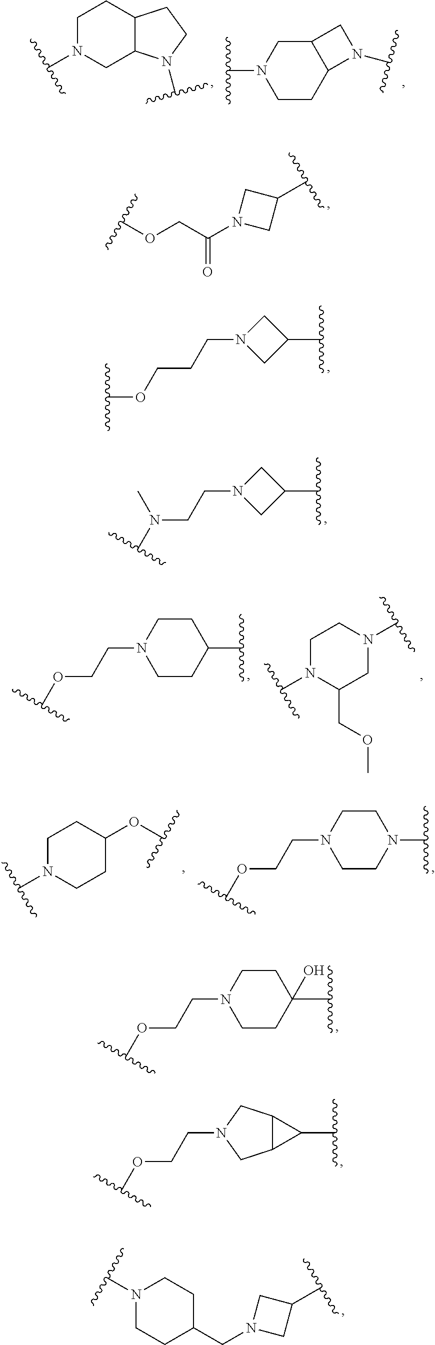

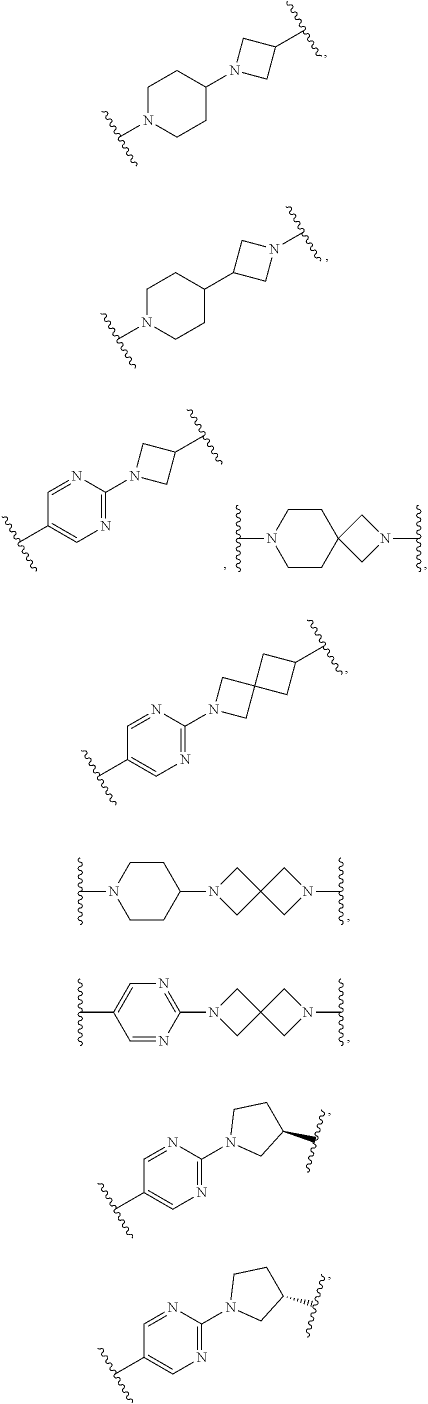

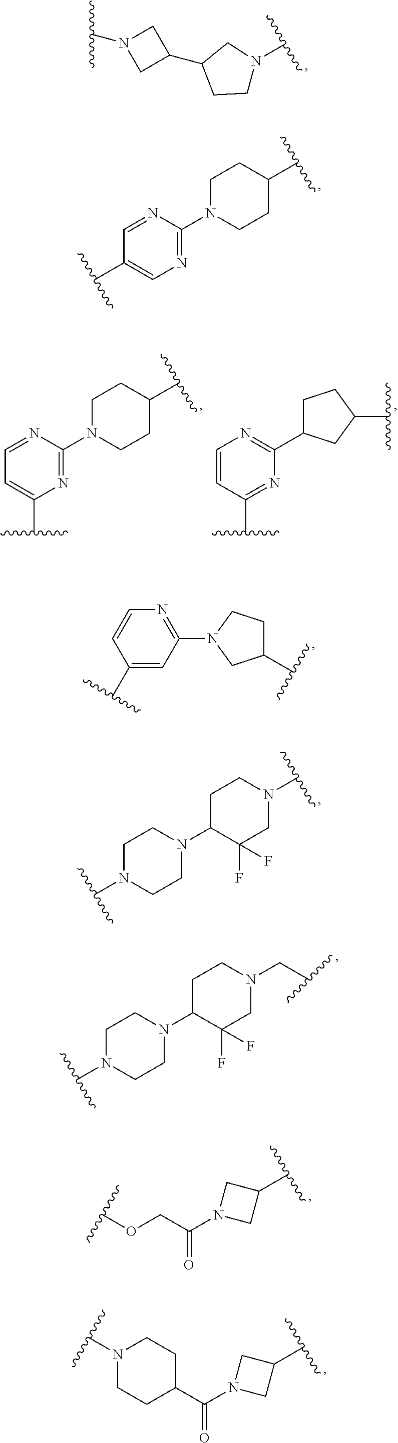

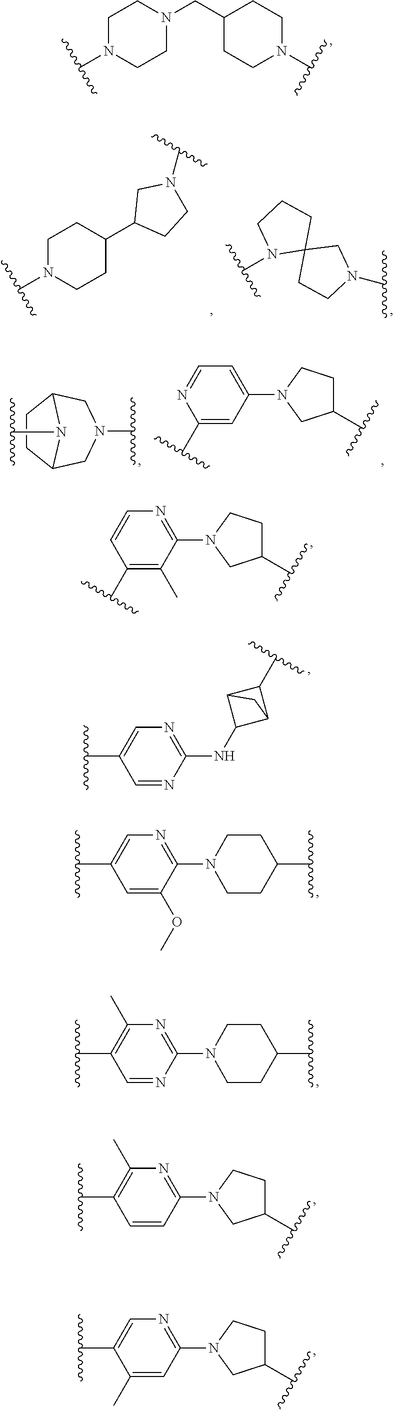

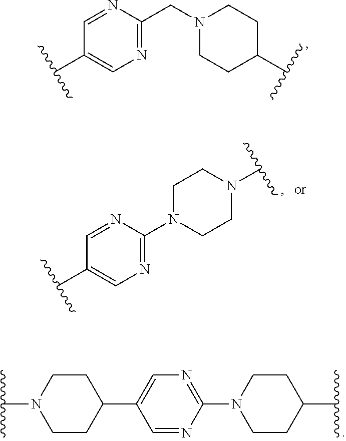 [SMILES: *c1cnc(CN2CCC(*)CC2)nc1 ; *c1cnc(N2CCN(*)CC2)nc1 ; *C1CCN(c2ncc(C3CCN(*)CC3)cn2)CC1]

50. The compound of claim 1, or a pharmaceutically acceptable salt thereof, wherein the linker has the structure of

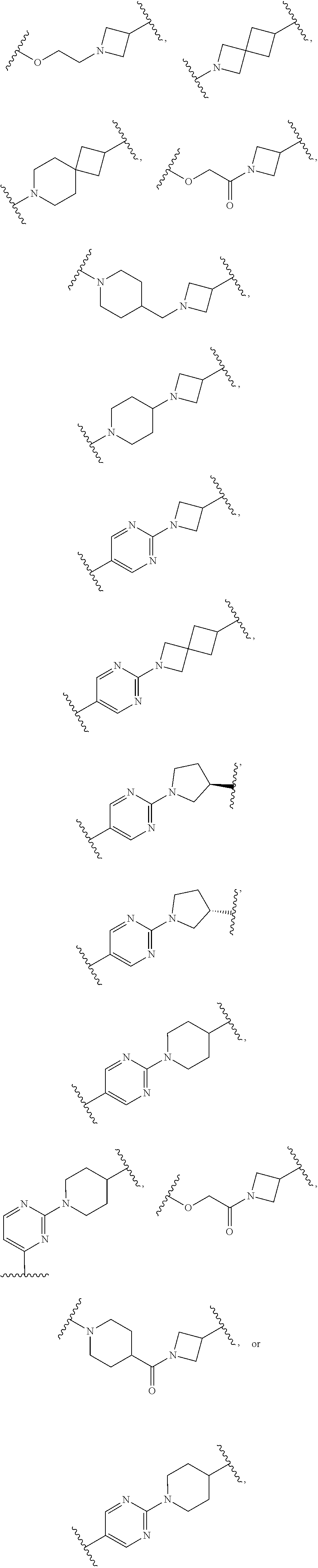

51. The compound of claim 1, or a pharmaceutically acceptable salt thereof, wherein the linker has the structure of Formula III:
A¹-(B¹)_{f}-(C¹)_{g}-(B²)ₕ-(B³)ᵢ-(C²)ⱼ-(B⁴)ₖ-A², Formula III
wherein
A¹ is a bond between the linker and ring system A;
A² is a bond between the degradation moiety and the linker;
each of B¹, B², B³, and B⁴ is, independently, optionally substituted ethynyl, optionally substituted C₆-C₁₀ aryl, optionally substituted C₃-C₁₀ cycloalkyl, optionally substituted C₃-C₁₀ carbocyclyl, optionally substituted C₂-C₁₀ heterocyclyl, optionally substituted C₂-C₉ heteroaryl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₁₀ heterocyclyl, optionally substituted C₆₋₁₂ aryl, or optionally substituted C₁₋₇ heteroalkyl;
each of C¹ and C² is, independently, carbonyl, thiocarbonyl, sulphonyl, or phosphoryl; and
each of f, g, h, i, j, and k is, independently, 0 or 1.

52. The compound of claim 51, or a pharmaceutically acceptable salt thereof, wherein the linker has the structure of

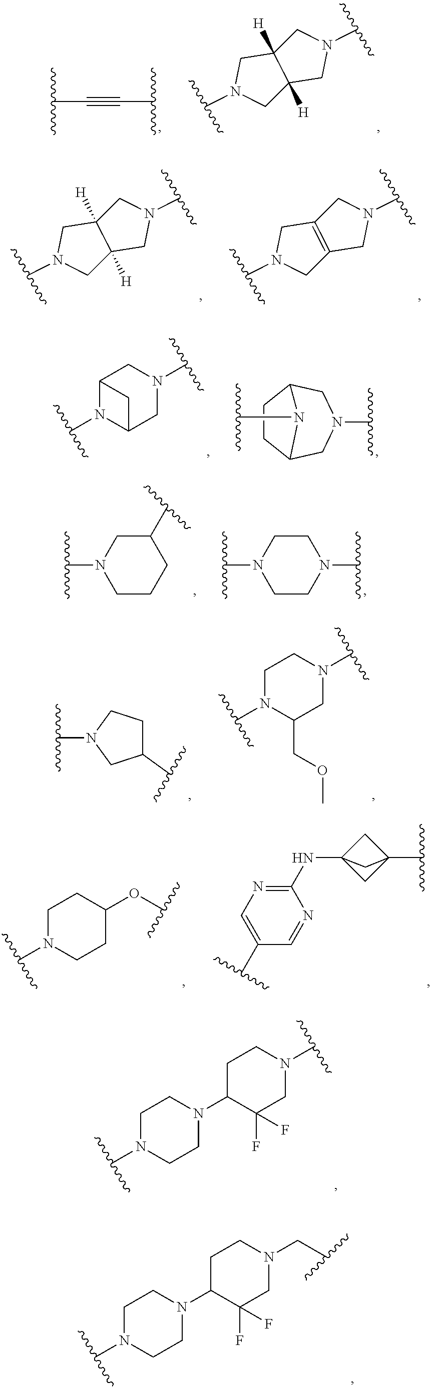

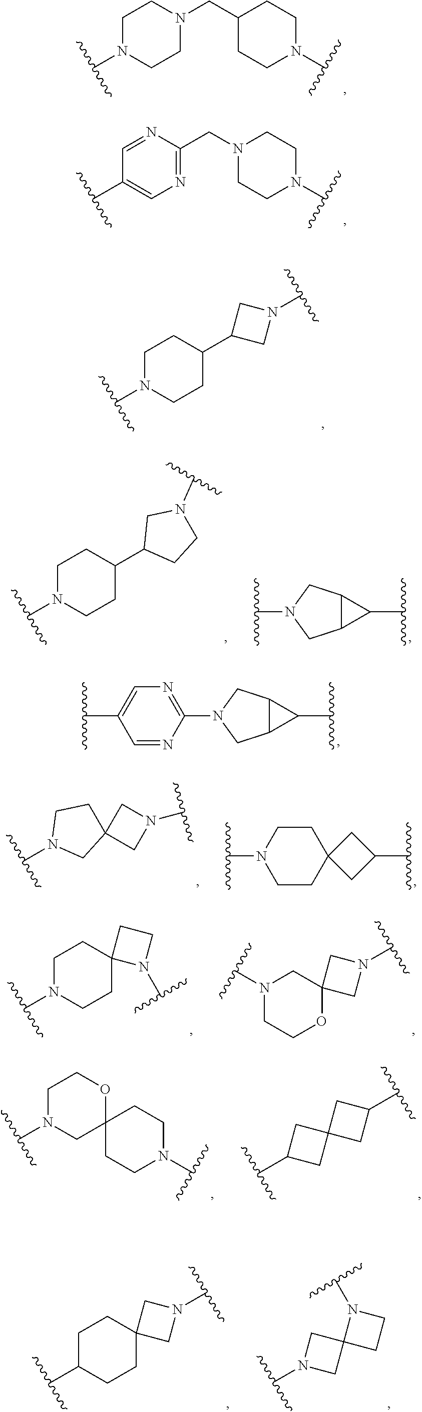

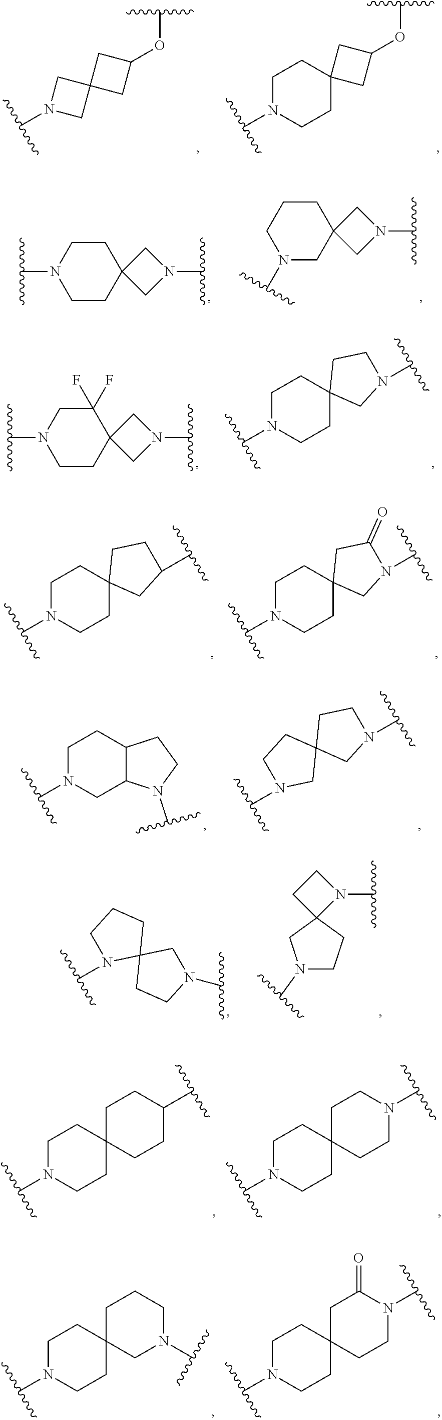

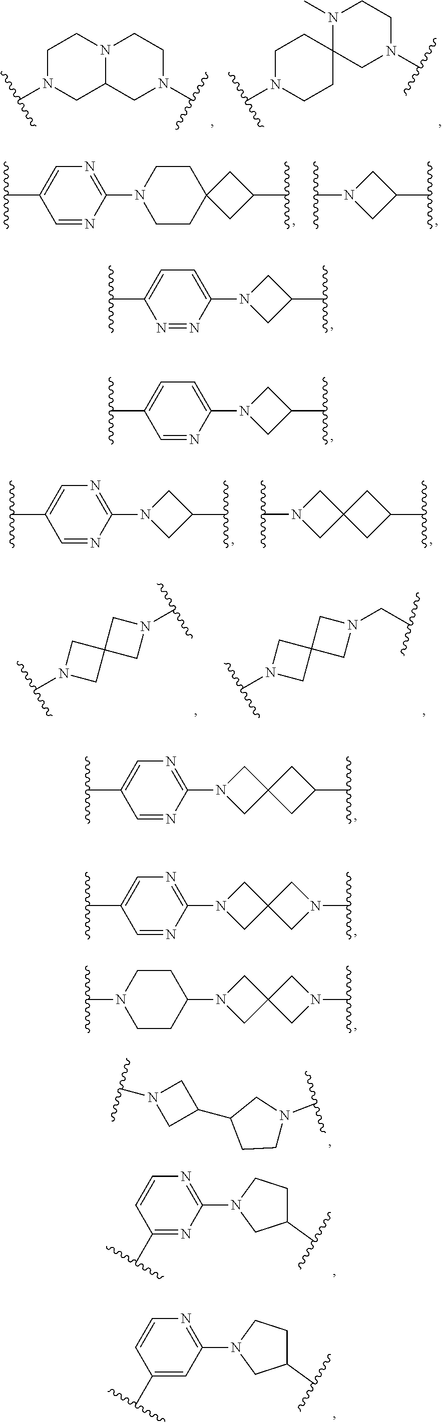

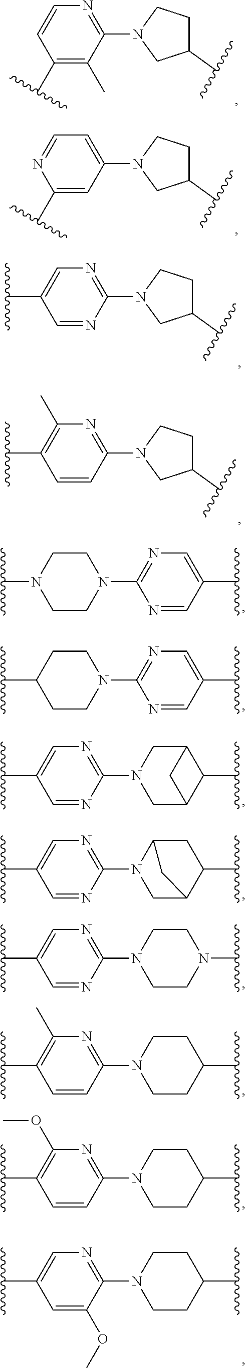

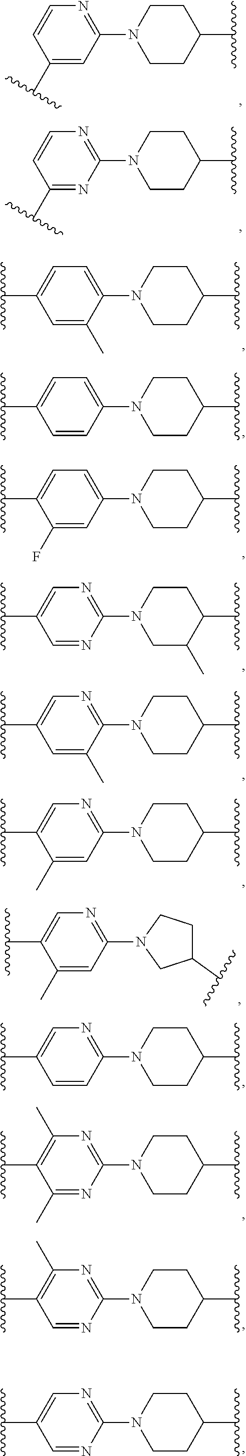

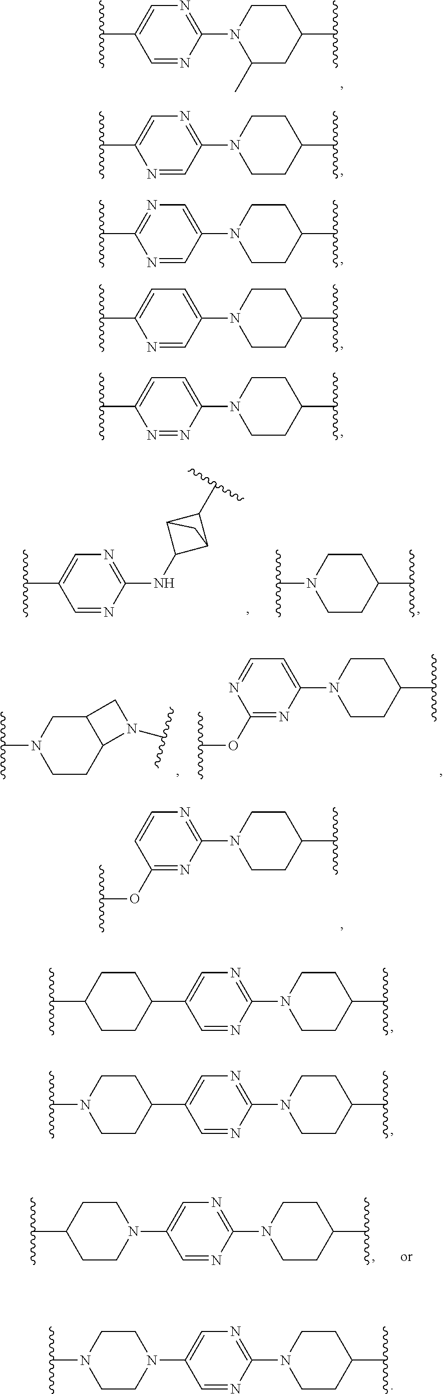

53. A pharmaceutical composition comprising a compound of claim 1 and a pharmaceutically acceptable excipient.

54. A method of treating a BAF complex-related disorder in a subject in need thereof, the method comprising administering to the subject an effective amount of a compound of claim 1, wherein the BAF complex-related disorder is cancer or a viral infection or a pharmaceutical composition.

55. (canceled)

56. A method of treating a disorder related to a BRG1 loss of function mutation in a subject in need thereof, the method comprising administering to the subject an effective amount of a compound of claim 1 or a pharmaceutical composition thereof, wherein the disorder related to a BRG1 loss of function mutation is cancer.

57. (canceled)

58. A method of treating cancer in a subject in need thereof, the method comprising administering to the subject an effective amount of a compound of claim 1 or a pharmaceutical composition thereof.

59. The method of claim 58, wherein the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

60.-69. (canceled)

70. A compound of the formula 1-121 in Table 1.
